(19) United States
(12) Patent Application Publication
(10) Pub. No.: US 2025/0388571 A1
(43) Pub. Date: Dec. 25, 2025

(54) AMP-ACTIVATED PROTEIN KINASE MODULATOR COMPOUNDS AND USES THEREOF

(71) Applicant: Biolexis Therapeutics, Inc., Lehi, UT (US)

(72) Inventors: Hariprasad Vankayalapati, Sandy, UT (US); Zhaoliang Li, Salt Lake City, UT (US); Chenyu Lin, Lehi, UT (US); Wendy Alejandra Cisneros Cancino, Lehi, UT (US); Kyle Medley, American Fork, UT (US); David J. Bearss, Alpine, UT (US)

(21) Appl. No.: 19/242,621

(22) Filed: Jun. 18, 2025

Related U.S. Application Data
(60) Provisional application No. 63/662,310, filed on Jun. 20, 2024.

(51) Int. Cl. C07D 413/04; A61K 31/4178; A61K 31/4184; A61K 31/422; A61K 31/4245; A61K 31/427; A61K 31/433; A61K 31/437; A61K 31/4439; A61K 31/675; A61K 31/683; A61K 31/69; A61P 3/04; A61P 3/10; C07D 403/04; C07D 403/12; C07D 405/04; C07D 405/12; C07D 413/12; C07D 413/14; C07D 417/04; C07D 471/04; C07F 5/02; C07F 9/572; C07F 9/6558; C07F 9/6561; C07F 9/6574

(52) U.S. Cl. CPC C07D 413/04; A61K 31/4178; A61K 31/4184; A61K 31/422; A61K 31/4245; A61K 31/427; A61K 31/433; A61K 31/437; A61K 31/4439; A61K 31/675; A61K 31/683; A61K 31/69; A61P 3/04; A61P 3/10; C07D 403/04; C07D 403/12; C07D 405/04; C07D 405/12; C07D 413/12; C07D 413/14; C07D 417/04; C07D 471/04; C07F 5/025; C07F 9/5728; C07F 9/65583; C07F 9/6561; C07F 9/65742

(57) ABSTRACT

Compounds having activity as modulators of AMPK are provided. The compounds have Structure (I), (II), (III), or (IV):

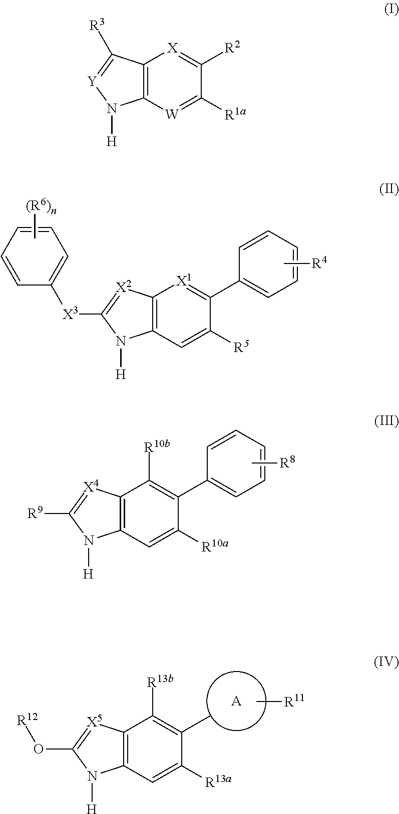

or a pharmaceutically acceptable salt, stereoisomer, or tautomer thereof, wherein R^{1a}, R², R³, W, X, Y, R⁴, R⁵, R⁶, X¹, X², X³, n, R⁸, R⁹, R^{10a}, R^{10b}, X⁴, ring A, R¹¹, R¹², R^{13a}, R^{13b}, and X⁵ are as defined herein. Methods associated with preparation and use of such compounds, pharmaceutical compositions comprising such compounds and methods to modulate the activity of AMPK are also provided.

### CROSS-REFERENCE TO RELATED APPLICATION

This application claims the benefit of U.S. Provisional Patent Application Ser. No. 63/662,310, filed Jun. 20, 2024, entitled, “AMP-ACTIVATED PROTEIN KINASE MODULATOR COMPOUNDS AND USES THEREOF,” the entirety of which is hereby incorporated by reference.

### BACKGROUND

### Technical Field

Embodiments of the present disclosure are generally directed to compounds and methods for their preparation and use as therapeutic or prophylactic agents, for example for treatment of metabolic diseases and/or neurodegenerative diseases.

### Description of Related Art

Adenosine monophosphate-activated protein kinase or AMP-activated protein kinase (AMPK) is a highly conserved serine/threonine protein kinase that acts as a central regulator of cellular energy homeostasis in eukaryotic cells. It is often referred to as a cellular energy sensor because its activation is triggered by a decrease in cellular ATP levels and an increase in the AMP to ATP ratio, indicating a state of cellular energy depletion. AMPK exists as a heterotrimeric protein complex composed of three subunits: a catalytic a subunit and regulatory β and γ subunits. Each subunit has multiple isoforms encoded by distinct genes, resulting in a diverse array of AMPK complexes with tissue-specific functions.

When cellular energy levels are low, AMPK becomes activated through a two-step process. An increase in AMP levels allosterically activates AMPK by binding to its γ subunit, promoting conformational changes that make the enzyme more sensitive to activation by upstream kinases, and phosphorylation of the a subunit at threonine-172 (Thr172) by upstream kinases, such as liver kinase B1 (LKB1) or calcium/calmodulin-dependent protein kinase kinase 2 (CaMKK2), further enhances AMPK activity.

Once activated, AMPK phosphorylates a wide range of downstream targets to restore cellular energy balance and promote energy conservation and production. Some of the key cellular processes regulated by AMPK include glucose uptake and glycolysis in skeletal muscle and other tissues while inhibiting glucose production in the liver. It promotes glucose transporter translocation to the cell membrane and phosphorylates enzymes involved in glycolysis and gluconeogenesis. AMPK stimulates fatty acid oxidation and inhibits lipogenesis, leading to reduced lipid accumulation in tissues. It phosphorylates and inactivates key enzymes involved in fatty acid synthesis and promotes the activation of enzymes involved in fatty acid oxidation and mitochondrial biogenesis.

AMPK inhibits protein synthesis by phosphorylating and inhibiting components of the mammalian target of rapamycin complex 1 (mTORC1) signaling pathway. This regulation helps conserve energy during times of cellular stress and nutrient deprivation. AMPK promotes mitochondrial biogenesis by phosphorylating transcriptional coactivators such as peroxisome proliferator-activated receptor gamma coactivator 1-alpha (PGC-1α), which in turn activates the transcription of genes involved in mitochondrial biogenesis and oxidative metabolism.

AMPK activation has implications for various physiological processes and diseases, including metabolism, cellular stress response, aging, obesity, type-2 diabetes, cardiometabolic, neurodegenerative diseases, and cancers. AMPK has emerged as an attractive target for the development of therapeutic interventions aimed at treating metabolic disorders and other related conditions.

Accordingly, there is a need to develop modulators (e.g., activators) that will directly target AMPK in several diseases, such as obesity, diabetes (e.g., type-2 diabetes), chronic inflammation, cardiometabolic diseases (e.g., cardiac energy homeostasis, ischemia-reperfusion injury, endothelial dysfunction, dyslipidemia, cardiac hypertrophy and remodeling), neuromuscular disorders (e.g., Duchenne muscular dystrophy), myotonic dystrophy type 1 (DM1), spinal muscular atrophy (SMA), non-alcoholic fatty liver disease (NAFLD), neurodegenerative diseases (e.g., Alzheimer's disease), and/or cancer. Embodiments of the present disclosure fulfill this need and provide further related advantages.

### BRIEF SUMMARY

In brief, embodiments of the present disclosure provide compounds, including pharmaceutically acceptable salts, stereoisomers, and tautomers thereof, which are capable of modulating AMPK.

In one aspect, the disclosure provides compounds have Structure (I), (II), (III), or (IV):

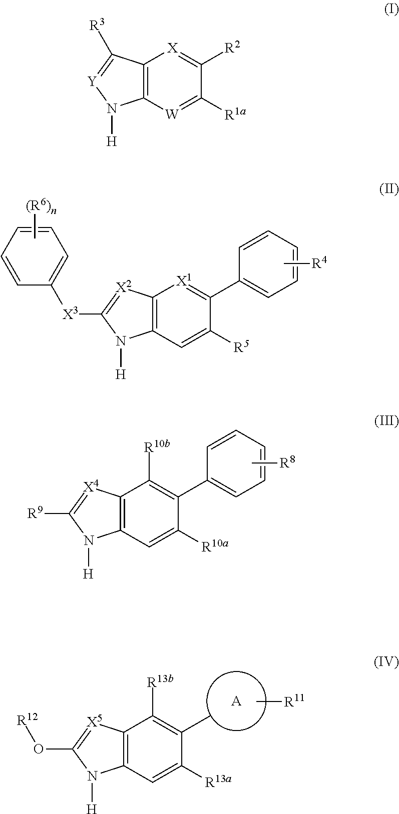

or a pharmaceutically acceptable salt, stereoisomer, or tautomer thereof, wherein R^{1a}, R², R³, W, X, Y, R⁴, R⁵, R⁶, X¹, X², X³, n, R⁸, R⁹, R^{10a}, R^{10b}, X⁴, ring A, R¹¹, R¹², R^{13a}, R^{13b} and X⁵ are as defined below.

In another aspect, pharmaceutical compositions comprising the disclosed compounds, and methods of use of the same for treatment of diseases (e.g., metabolic diseases, cancers, and/or neurodegenerative diseases) are also provided.

### BRIEF DESCRIPTION OF THE DRAWINGS

In the figures, identical reference numbers identify similar elements. The sizes and relative positions of elements in the figures are not necessarily drawn to scale. For example, the shapes of various elements and angles are not drawn to scale and some of these elements are enlarged and positioned to improve figure legibility. Further, the shapes of the elements as drawn, are not intended to convey any information regarding the actual shape of the elements and have been solely selected for ease of recognition in the figures.

FIGS. 1-4 show results for cells treated with differing doses of representative compounds of Structure (I) (i.e., compound I-11 in FIG. 1 and compound I-7 in FIG. 2) as well reference compounds (i.e., PF-06409577 in FIG. 3 and MK-8722 in FIG. 4). Compounds I-11 and I-7 show dose dependence for activation of insulin in human skeletal muscle cells (HSKM cells). Reference compounds show that AMPK activation that is highly variable when compared to compounds of Structure (I). Cells were starved for 3 hours in high glucose DMEM without FBS, then treated with indicated compounds in DMEM without FBS/glucose for 1 hour, and glucose uptake was detected using an uptake glucose kit (Promega). The bars show (from left to right) concentrations of 100 μM, 33 μM, 11 μM, 3.7 μM, 1.23 μM, 0.41 μM, 0.14 μM, 0.045 μM, 0.015 μM, 0.005 μM, 0 μM, and insulin.

FIGS. 5-8 show results for glucose update assays in Human white preadipocyte (HWP) cells (Promocell, cat. No. C-12735) when treated with compound I-11 (FIG. 5), compound I-7 (FIG. 6), MK-8722 (FIG. 7), and PF-06409577 (FIG. 8). HWP cells (subcutaneous upper arm of female Caucasian obese patient) were starved for 3 hours in DMEM without FBS, treated with the components noted above in high glucose DMEM without FBS/glucose for 1 hour, and glucose uptake was detected using a glucose uptake kit (Promega). It was unexpectedly discovered that compound I-11 activates insulin in a dose dependent manner. The bars show (from left to right) concentrations of 100 μM, 33 μM, 11 μM, 3.7 μM, 1.23 μM, 0.41 μM, 0.14 μM, 0.045 μM, 0.015 μM, 0.005 μM, 0 μM, and insulin.

FIGS. 9-12 show results for glucose update assays in Human white preadipocyte (HWP) cells (Promocell, cat. No. C-12735) when treated with compound I-11 (FIG. 9), compound I-7 (FIG. 10), MK-8722 (FIG. 11), and PF-06409577 (FIG. 12). HWP cells (female abdomen cells) were starved for 3 hours in DMEM without FBS, treated with the components noted above in high glucose DMEM without FBS/glucose for 1 hour, and glucose uptake was detected using a glucose uptake kit (Promega). It was unexpectedly discovered that compound I-7 activates insulin in a dose dependent manner. The bars show (from left to right) concentrations of 100 μM, 33 μM, 11 μM, 3.7 μM, 1.23 μM, 0.41 μM, 0.14 μM, 0.045 μM, 0.015 μM, 0.005 μM, 0 μM, and insulin.

FIGS. 13-16 show results for glucose update assays in THLE-2 cells (ATCC, cat. No. CRL-2706) when treated with compound I-11 (FIG. 13), compound I-7 (FIG. 14), MK-8722 (FIG. 15), and PF-06409577 (FIG. 16). The THLE-2 cells were starved for 3 hours in high glucose DMEM without FBS, treated with the components noted above in DMEM without FBS/glucose for 1 hour, and glucose uptake was detected using a glucose uptake kit (Promega). The bars show (from left to right) concentrations of 100 μM, 33 μM, 11 μM, 3.7 μM, 1.23 μM, 0.41 μM, 0.14 μM, 0.045 μM, 0.015 μM, 0.005 μM, 0 μM, and insulin.

FIGS. 17-19 show results for glucose update assays in human cardiomyocyte cells (“HCM”-healthy donor) (Promocell, cat. no. C-12810) when treated with compound I-11 (FIG. 17), compound I-7 (FIG. 18), and PF-06409577 (FIG. 19). After recovered HCM cells for 4 days, cells were starved for 3 hours in high glucose DMEM without FBS, treated with the components noted above in DMEM without FBS/glucose for 1 hour, and glucose uptake was detected using a glucose uptake kit (Promega). It was unexpectedly discovered that compounds of Structure (I) did not show AMPK activation activity in healthy donor cardiomyocyte cells, which indicates that activation activity is HSKM cell specific and indicates a good safety profile for healthy HCM cells. The reference compound (i.e., PF-06409577) demonstrated significant variability at the 10 doses that were tested. The bars show (from left to right) concentrations of 100 μM, 33 μM, 11 μM, 3.7 μM, 1.23 μM, 0.41 μM, 0.14 μM, 0.045 μM, 0.015 μM, 0.005 μM, 0 μM, and insulin.

FIGS. 20-22 show results for glucose update assays in human cardiomyocyte cells (“HCM”-diabetic donor) (Promocell, cat. no. C-12810) when treated with compound I-11 (FIG. 20), compound I-7 (FIG. 21), and PF-06409577 (FIG. 22). After recovered HCM cells for 4 days, cells were starved for 3 hours in high glucose DMEM without FBS, treated with the components noted above in DMEM without FBS/glucose for 1 hour, and glucose uptake was detected using a glucose uptake kit (Promega). It was unexpectedly discovered that compounds of Structure (I) did not show AMPK activation activity in healthy donor cardiomyocyte cells, which indicates that activation activity is HSKM cell specific and indicates a good safety profile for diabetic HCM cells. The reference compound (i.e., PF-06409577) demonstrated significant variability at the 10 doses that were tested. The bars show (from left to right) concentrations of 100 μM, 33 μM, 11 μM, 3.7 μM, 1.23 μM, 0.41 μM, 0.14 μM, 0.045 μM, 0.015 μM, 0.005 μM, 0 μM, and insulin.

FIG. 23 shows the effect compound I-11 in db/db mice on fasted blood glucose at day 28. From left to right the columns show vehicle alone, group 2 (G2) treated with compound I-11 at 30 mg/kg (po, QD) and group 3 (G3) treated with compound I-11 at 50 mg (po, QD). Data is shown as Mean±S.E.M. (n=10), *P<0.5 and ***P<0.001, Significant difference as compared to vehicle Control (10 mL/kg); One-way ANOVA followed by Dunnett's multiple comparisons test.

FIG. 24 shows animals fed blood glucose at day 14 when treated with (from left to right) 60% high fat diet (HFD) when treated with vehicle control (p.o.; q.d.—Group 1 or G1), 60% HFD when treated with compound I-11 (at 30 mg/kg, p.o.; q.d.—Group 2 or G2), and 60% HFD when treated with compound I-11 (at 50 mg/kg, p.o.; q.d.—Group 3 or G3).

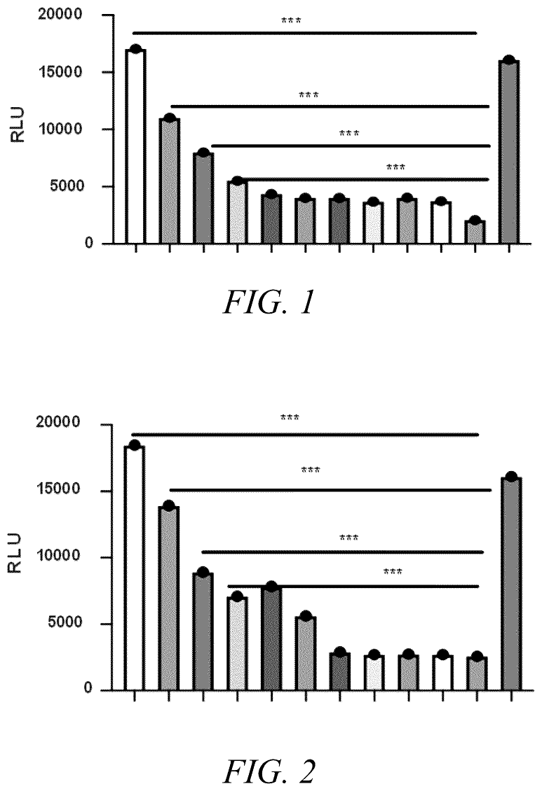

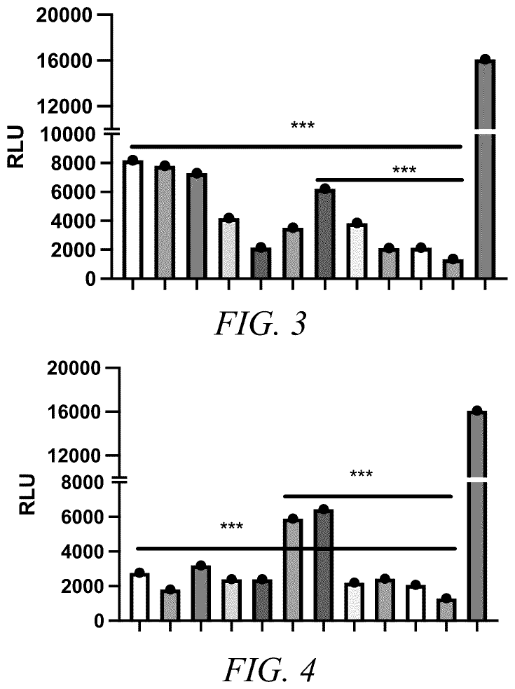

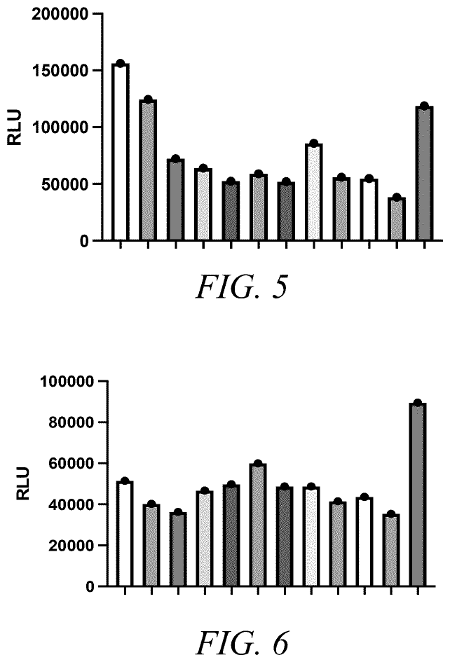

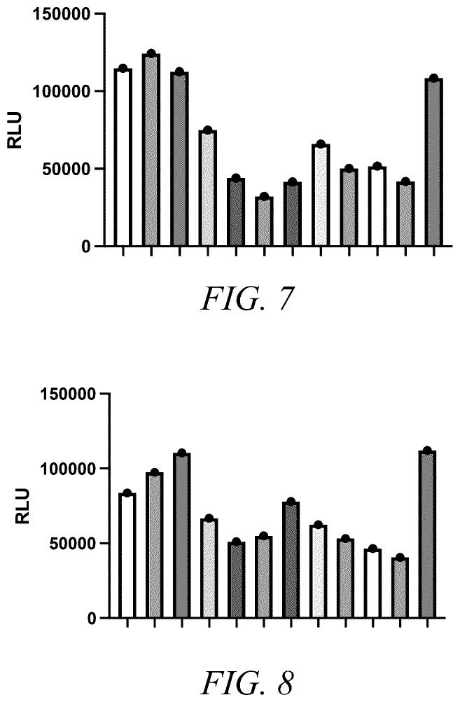

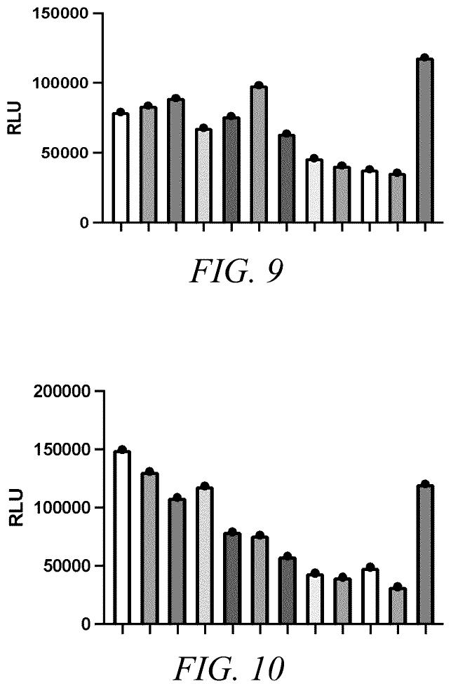

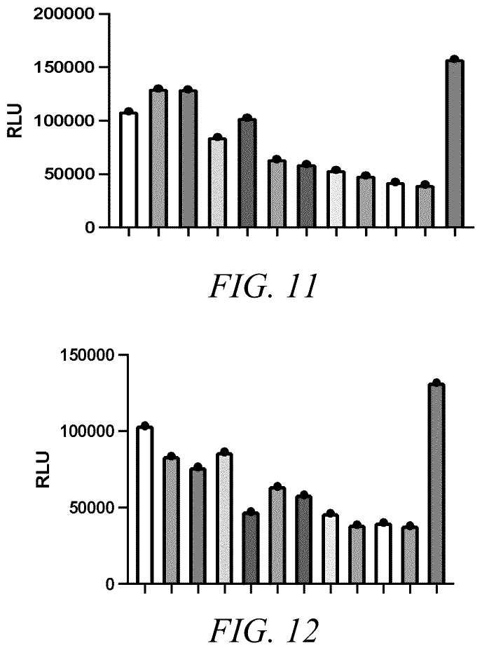

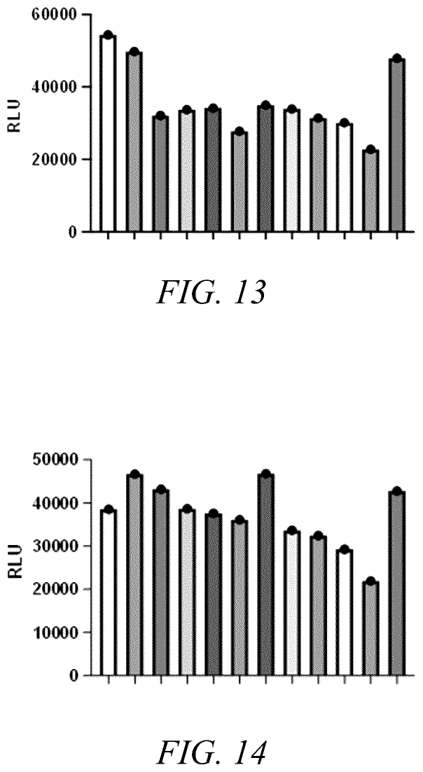

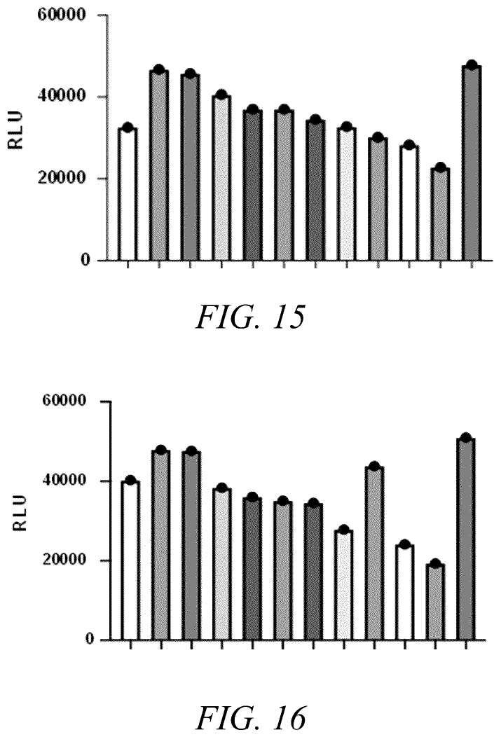

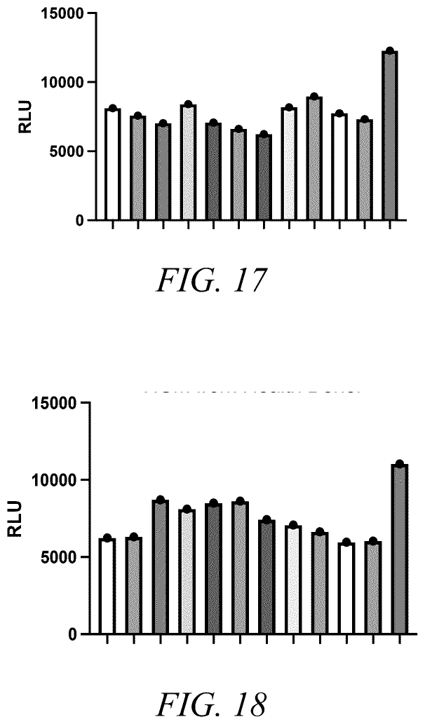

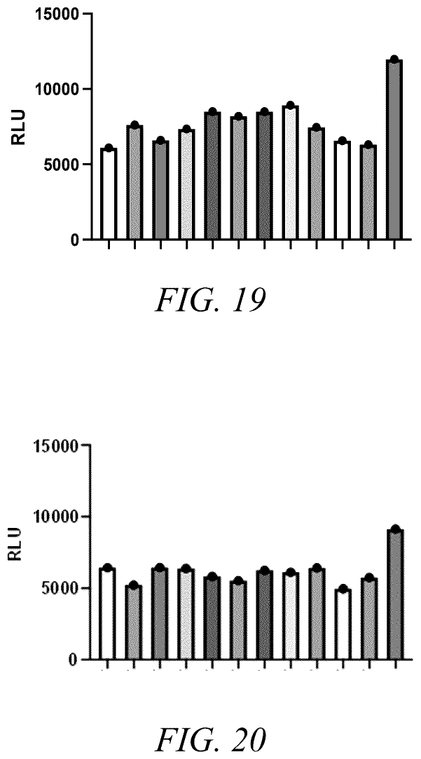

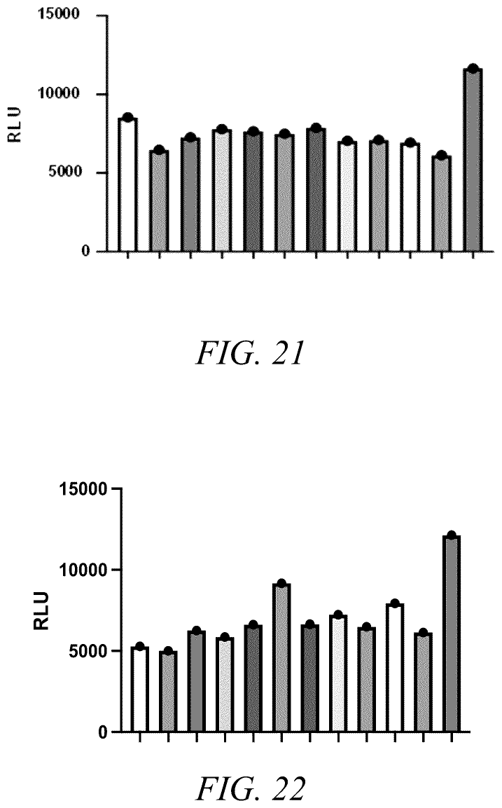

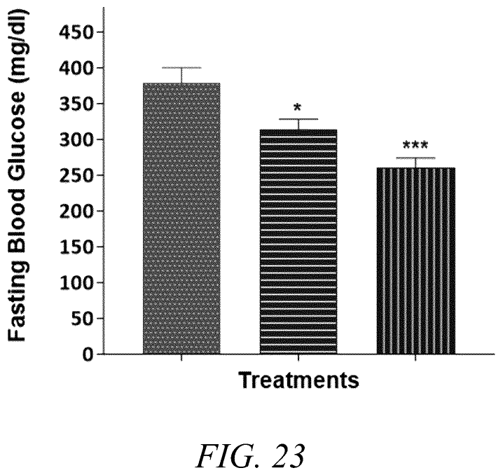

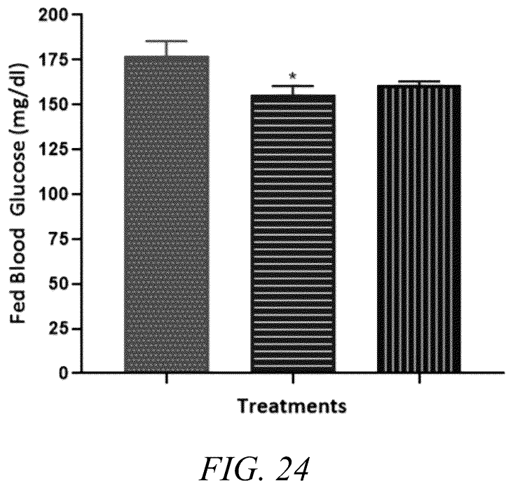

### DETAILED DESCRIPTION

In the following description, certain specific details are set forth to provide a thorough understanding of various embodiments of the disclosure. However, one skilled in the art will understand that the disclosure may be practiced without these details.

Unless the context requires otherwise, throughout the present specification and claims, the word “comprise” and variations thereof, such as, “comprises” and “comprising” are to be construed in an open, inclusive sense, that is, as “including, but not limited to”.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, the terms “about” and “approximately” mean±20%, ±10%, ±5% or ±1% of the indicated range, value, or structure, unless otherwise indicated. The terms “a” and “an” as used herein refer to “one or more” of the enumerated components. The use of the alternative (e.g., “or”) should be understood to mean either one, both, or any combination thereof of the alternatives.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the features, structures, or characteristics may be combined in any suitable manner in one or more embodiments.

Unless defined otherwise, all technical and scientific terms used herein have the same meaning as is commonly understood by one of skill in the art to which this disclosure belongs. As used in the specification and claims, the singular form “a”, “an” and “the” include plural references unless the context clearly dictates otherwise.

“Amino” refers to the —NH₂ radical.

“Cyano” refers to the —CN radical.

“Hydroxy” or “hydroxyl” refers to the —OH radical.

“Oxo” refers to the ═O substituent.

“Alkyl” refers to a saturated, straight or branched hydrocarbon chain radical consisting solely of carbon and hydrogen atoms, having from one to twelve carbon atoms (C₁-C₁₂ alkyl), one to eight carbon atoms (C₁-C₈ alkyl) or one to six carbon atoms (C₁-C₆ alkyl), or any value within these ranges, such as C₄-C₆ alkyl and the like, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl and the like. The number of carbons referred to relates to the carbon backbone and carbon branching but does not include carbon atoms belonging to any substituents. Unless stated otherwise specifically in the specification, an alkyl group is optionally substituted.

“Alkoxy” refers to a radical of the formula —ORₐ where Rₐ is an alkyl radical as defined above containing one to twelve carbon atoms (C₁-C₁₂ alkoxy), one to eight carbon atoms (C₁-C₈alkoxy) or one to six carbon atoms (C₁-C₆ alkoxy), or any value within these ranges. Unless stated otherwise specifically in the specification, an alkoxy group is optionally substituted.

“Aromatic ring” refers to a cyclic planar molecule or portion of a molecule (i.e., a radical) with a ring of resonance bonds that exhibits increased stability relative to other connective arrangements with the same sets of atoms. Generally, aromatic rings contain a set of covalently bound co-planar atoms and comprises a number of π-electrons (for example, alternating double and single bonds) that is even but not a multiple of 4 (i.e., 4n+2π-electrons, where n=0, 1, 2, 3, etc.). Aromatic rings include, but are not limited to, phenyl, naphthenyl, imidazolyl, pyrrolyl, pyridinyl, pyrimidinyl, pyrazinyl, pyridonyl, pyridazinyl, pyrimidonyl. Unless stated otherwise specifically in the specification, an “aromatic ring” includes all radicals that are optionally substituted.

“Aryl” refers to a carbocyclic ring system radical comprising 6 to 18 carbon atoms, for example 6 to 10 carbon atoms (C₆-C₁₀ aryl) and at least one carbocyclic aromatic ring. For purposes of embodiments of this disclosure, the aryl radical is a monocyclic, bicyclic, tricyclic, or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group is optionally substituted.

“Cycloalkyl” refers to a non-aromatic monocyclic or polycyclic carbocyclic radical consisting solely of carbon and hydrogen atoms, which may include fused or bridged ring systems, having from three to fifteen ring carbon atoms (C₃-C₁₅ cycloalkyl), from three to ten ring carbon atoms (C₃-C₁₀ cycloalkyl), or from three to eight ring carbon atoms (C₃-C₈ cycloalkyl), or any value within these ranges such as three to four carbon atoms (C₃-C₄ cycloalkyl), and which is saturated or partially unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group is optionally substituted.

“Fused” refers to any ring structure described herein which is fused to another ring structure.

“Halo” refers to bromo, chloro, fluoro, or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group is optionally substituted.

“Haloalkoxy” refers to a radical of the formula —ORₐ where Rₐ is an haloalkyl radical as defined above. Unless stated otherwise specifically in the specification, a haloalkoxy group is optionally substituted.

“Hydroxyalkyl” refers to an alkyl radical, as defined above that is substituted by one or more hydroxyl radical. The hydroxyalkyl radical is joined to the remainder of the molecule through an alkyl carbon atom. Unless stated otherwise specifically in the specification, a hydroxyalkyl group is optionally substituted.

“Aminoalkyl” refers to an alkyl radical, as defined above, that is substituted by one or more —NH₂ groups. The aminoalkyl radical is joined to the remainder of the molecule though an alkyl carbon atom. Unless stated otherwise specifically in the specification, an aminoalkyl group is optionally substituted.

“Heterocyclyl” refers to a 3- to 18-membered, for example 3- to 10-membered or 3- to 8-membered, non-aromatic ring radical having one to ten ring carbon atoms (e.g., two to ten) and from one to six ring heteroatoms selected from the group consisting of nitrogen, oxygen, and sulfur. Unless stated otherwise specifically in the specification, the heterocyclyl radical is partially or fully saturated and is a monocyclic, bicyclic, tricyclic, or tetracyclic ring system, which may include fused, spirocyclic and/or bridged ring systems. Nitrogen, carbon, and sulfur atoms in a heterocyclyl radical are optionally oxidized, and nitrogen atoms may be optionally quaternized. Examples of such heterocyclyl radicals include, but are not limited to, dioxolanyl, thienyl[1,3]dithianyl, decahydroisoquinolyl, furanonyl, imidazolinyl, imidazolidinyl, isothiazolidinyl, isoxazolidinyl, morpholinyl, octahydroindolyl, octahydroisoindolyl, hexahydro-1H-pyrrolizine, 2-oxopiperazinyl, 2-oxopiperidinyl, 2-oxopyrrolidinyl, oxazolidinyl, oxiranyl, piperidinyl, piperazinyl, 4-piperidonyl, azetidinyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. The terms “O-heterocyclyl” and “N-heterocyclyl” refer to a heterocyclyl as defined above comprising at least one ring oxygen (e.g., oxirane, oxetane, tetrahydrofuran, dioxane, etc.) or at least one ring nitrogen (e.g., aziridine, pyrrolidine, morpholine, etc.), respectively. Unless stated otherwise specifically in the specification, a heterocyclyl group is optionally substituted.

“Heteroaryl” refers to a 5- to 18-membered, for example 5- to 6-membered, ring system radical comprising one to thirteen ring carbon atoms, one to six ring heteroatoms selected from the group consisting of nitrogen, oxygen, and sulfur, and at least one aromatic ring. Heteroaryl radicals may be a monocyclic, bicyclic, tricyclic, or tetracyclic ring system, which may include fused or bridged ring systems; and the nitrogen, carbon, or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group is optionally substituted.

The term “substituted” as used herein means any of the above groups (e.g., alkyl, alkoxy, aryl, cycloalkyl, heterocyclyl, etc.) wherein at least one hydrogen atom (e.g., 1, 2, 3 or all hydrogen atoms) is replaced by a bond to a non-hydrogen substituent. Examples of non-hydrogen substituents include, but are not limited to amino, carboxyl, cyano, hydroxyl, halo, nitro, oxo, thiol, thioxo, alkyl, alkenyl, alkylcarbonyl, alkoxy, aryl, cyanoalkyl, cycloalkyl, haloalkyl, heterocyclyl, heterocyclylalkyl, heteroaryl, heteroarylalkyl and/or hydroxyalkyl substituents, each of which may also be optionally substituted with one or more of the above substituents.

The term “effective amount” or “therapeutically effective amount” refers to that amount of a compound described herein that is sufficient to affect the intended application including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the compounds chosen, the dosing regimen to be followed, whether it is administered in combination with other compounds, timing of administration, the tissue to which it is administered, and the physical delivery system in which it is carried.

As used herein, “treatment” or “treating” refer to an approach for obtaining beneficial or desired results with respect to a disease, disorder or medical condition including but not limited to a therapeutic effect and/or a prophylactic effect. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. A prophylactic effect includes delaying or eliminating the appearance of a disease or condition, delaying, or eliminating the onset of symptoms of a disease or condition, slowing, halting, or reversing the progression of a disease or condition, or any combination thereof. In certain embodiments, for prophylactic benefit, the compositions are administered to a subject at risk of developing a particular disease, or to a subject reporting one or more of the physiological symptoms of a disease, even though a diagnosis of this disease may not have been made.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

“Pharmaceutically acceptable salt” includes both acid and base addition salts.

“Pharmaceutically acceptable acid addition salt” refers to those salts which retain the biological effectiveness of the free bases, which are biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable acid addition salts are those that are pharmacologically effective and suitable for contact with the tissues of patients without undue toxicity, irritation, or allergic response. Pharmaceutically acceptable acid addition salts which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness of the free acids, which are biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable base addition salts are those that are pharmacologically effective and suitable for contact with the tissues of patients without undue toxicity, irritation, or allergic response. Pharmaceutically acceptable base addition salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

In some embodiments, pharmaceutically acceptable salts include quaternary ammonium salts such as quaternary amine alkyl halide salts (e.g., methyl bromide).

The term “inhibitor” refers to a compound having the ability to inhibit a biological function of a target protein, whether by inhibiting the activity or expression of the protein. Accordingly, the term “inhibitor” is defined in the context of the biological role of the target protein. In some embodiments, inhibitors specifically interact with (e.g., bind to) a target. In some embodiments, the biological activity inhibited is the development, growth, or spread of a tumor.

“Subject” refers to an animal, such as a mammal, for example a human. The methods described herein can be useful in both human therapeutics and veterinary applications. In some embodiments, the subject is a mammal, and in some embodiments, the subject is human.

“Mammal” includes humans and both domestic animals such as laboratory animals and household pets (e.g., cats, dogs, swine, cattle, sheep, goats, horses, rabbits), and non-domestic animals such as wildlife and the like.

“Prodrug” is meant to indicate a compound that may be converted under physiological conditions or by solvolysis to a biologically active compound described herein (e.g., compounds of Structure (I), (II), (III), or (IV)). Thus, the term “prodrug” refers to a precursor of a biologically active compound that is pharmaceutically acceptable. In some embodiments, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or thiol group is bonded to any group that, when the prodrug of the active compound is administered to a mammalian subject, cleaves to form a free hydroxy, free amino or free mercapto group, respectively. Examples of prodrugs include, but are not limited to, acetate, formate, and benzoate derivatives of a hydroxy functional group, or acetamide, formamide and benzamide derivatives of an amine functional group in the active compound and the like.

The term “in vivo” refers to an event that takes place in a subject's body.

Embodiments disclosed herein are also meant to encompass all pharmaceutically acceptable compounds of Structure (I), (II), (III), or (IV).

Certain embodiments are also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, embodiments include compounds produced by a process comprising administering a compound of this disclosure to a mammal for a period sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

Often crystallizations produce a solvate of the compounds disclosed herein. As used herein, the term “solvate” refers to an aggregate that comprises one or more compounds of the disclosure with one or more molecules of solvent. In some embodiments, the solvent is water, in which case the solvate is a hydrate. Alternatively, in other embodiments, the solvent is an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. In some embodiments, the compounds of the disclosure are a true solvate, while in other cases, the compounds of the disclosure merely retain adventitious water or is a mixture of water plus some adventitious solvent.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

A “pharmaceutical composition” refers to formulations of compounds of the disclosure and a medium generally accepted in the art for the delivery of compounds of the disclosure to mammals, e.g., humans. Such a medium includes all pharmaceutically acceptable carriers, diluents, or excipients therefor.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

The compounds of the disclosure (i.e., compounds of Structure (I), (II), (III), or (IV)) or their pharmaceutically acceptable salts may contain one or more centers of geometric asymmetry and may thus give rise to stereoisomers such as enantiomers, diastereomers, and other stereoisomeric forms that are defined, in terms of absolute stereochemistry, as (R)- or (S) or, as (D)- or (L)- for amino acids. Embodiments thus include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

Embodiments of the present disclosure include all manner of rotamers and conformationally restricted states of a compound of the disclosure. Atropisomers, which are stereoisomers arising because of hindered rotation about a single bond, where energy differences due to steric strain or other contributors create a barrier to rotation that is high enough to allow for isolation of individual conformers, are also included. As an example, certain compounds of the disclosure may exist as mixtures of atropisomers or purified or enriched for the presence of one atropisomer.

In some embodiments, the compounds of Structure (I), (II), (III), or (IV) are a mixture of enantiomers or diastereomers. In other embodiments, the compounds of Structure (I), (II), (III), or (IV) are substantially one enantiomer or diastereomer.

A “tautomer” refers to a proton shift from one atom of a molecule to another atom of the same molecule. Embodiments thus include tautomers of the disclosed compounds.

The chemical naming protocol and structure diagrams used herein are a modified form of the I.U.P.A.C. nomenclature system, using the ACD/Name Version 9.07 software program and/or ChemDraw Professional Version 17.0.0.206 software naming program (CambridgeSoft). For complex chemical names employed herein, a substituent group is typically named before the group to which it attaches. For example, cyclopropylethyl comprises an ethyl backbone with a cyclopropyl substituent. Except as described below, all bonds are identified in the chemical structure diagrams herein, except for all bonds on some carbon atoms, which are assumed to be bonded to sufficient hydrogen atoms to complete the valency.

“MK-8722” (or similar) is an AMPK activator catalogued under CAS Registry No. 1394371-71-1 and having the following structure:

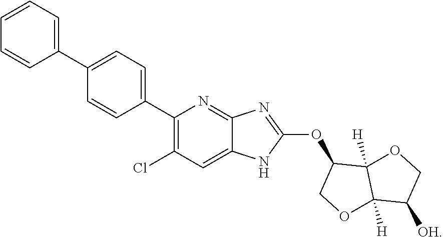 [SMILES: O[C@@H]1CO[C@H]2[C@@H]1OC[C@H]2Oc1nc2nc(-c3ccc(-c4ccccc4)cc3)c(Cl)cc2[nH]1]

“PF-06409577” (or similar) is an AMPK activator catalogued under CAS Registry No. 1467057-23-3 and having the following structure:

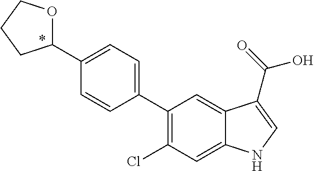 [SMILES: O=C(O)c1c[nH]c2cc(Cl)c(-c3ccc(C4CCCO4)cc3)cc12]

### Compounds

The disclosure provides compounds including pharmaceutically acceptable salts, stereoisomers, and tautomers thereof, which are capable of modulating AMPK.

Accordingly, one embodiment provides a compound having the following Structure (I):

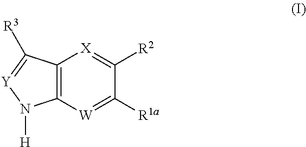

or a pharmaceutically acceptable salt, stereoisomer, or tautomer thereof, wherein:
Y is N or CH;
X is N or CR^{1b};
W is N or CR^{1c};
R^{1a}, R^{1b}, and R^{1c} are each independently hydrogen or halo;
R² is 3-8 membered N-heterocyclyl, —NH—R^{2d}, or has the following structure:

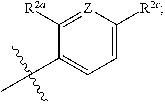

Z is N or CR^{2b};
R^{2a}, R^{2b}, and R^{2c} are each independently hydrogen, optionally substituted C₁-C₄ alkoxy, —(CH₂)ₙ₁O(CH₂)ₙ₂O(CH₂)ₙ₃OH, —N═S(CH₃)₂═O, optionally substituted C₃-C₈ cycloalkyl, or optionally substituted 3-8 membered heterocyclyl;
R^{2d} has one of the following structures:

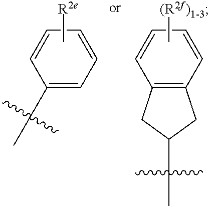

R^{2e} is optionally substituted C₃-C₆ cycloalkyl;
R^{2f} is halo or optionally substituted C₃-C₆ cycloalkyl;
n1, n2, and n3 are each independently 1, 2, 3, or 4; and
R³ has one of the following structures:

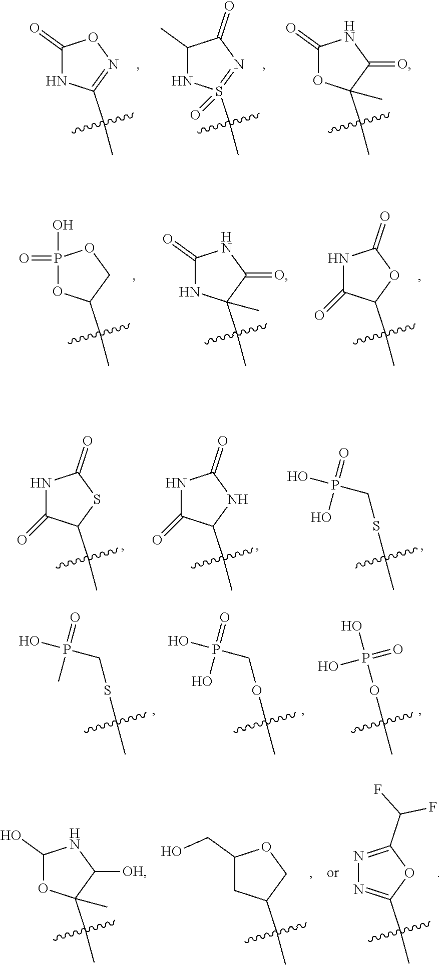

One embodiment provides a compound having the following Structure (I):

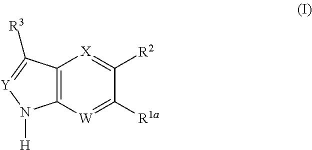

or a pharmaceutically acceptable salt, stereoisomer, or tautomer thereof, wherein:
Y is N or CH;
X is N or CR^{1b};
W is N or CR^{1c};
R^{1a}, R^{1b}, and R^{1c} are each independently hydrogen or halo;
R² is 3-8 membered N-heterocyclyl, —NH—R^{2d}, or has the following structure:

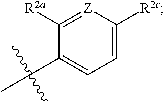

Z is N or CR^{2b};
R^{2a}, R^{2b}, and R^{2c} are each independently hydrogen, optionally substituted C₁-C₄ alkoxy, —(CH₂)ₙ₁O(CH₂)ₙ₂O(CH₂)ₙ₃OH, —N═S(CH₃)₂═O, optionally substituted C₃-C₈ cycloalkyl, or optionally substituted 3-8 membered heterocyclyl;
R^{2d} has one of the following structures:

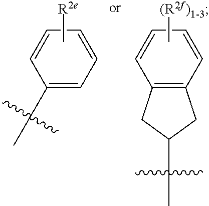

R^{2e} is optionally substituted C₃-C₆ cycloalkyl;
R^{2f} is halo or optionally substituted C₃-C₆ cycloalkyl;
n1, n2, and n3 are each independently 1, 2, 3, or 4; and
R³ has one of the following structures:

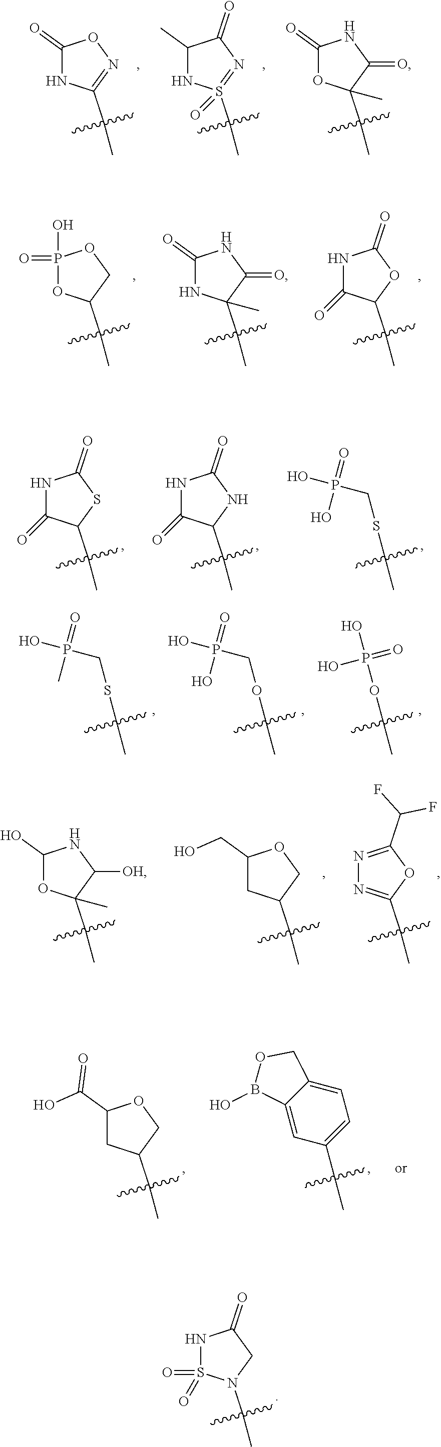

In some embodiments, Y is N. In certain embodiments, Y is CH. In some embodiments, X is N. In certain embodiments, X is CH, or CF. In some embodiments, W is N or CH.

In certain embodiments, R^{1a} is hydrogen, fluoro, or chloro.

In some embodiments, R² is a monocyclic 5-6 membered N-heterocyclyl. In some embodiments, R² is pyrrolidinyl. In certain embodiments, R² is optionally substituted with one or more —OH substituents. In certain embodiments, R² has one of the following structures:

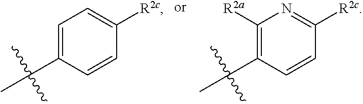

In some embodiments, R^{2a} is methoxy. In certain embodiments, R^{2c} is C₃-C₆ cycloalkyl optionally substituted with one or more —OH substituents. In some embodiments, R^{2c} is a 3-6 membered O-heterocyclyl or a 3-6 membered N-heterocyclyl each being optionally substituted with one or more —OH substituents. In some embodiments, R^{2a}, R^{2c}, or both are independently methoxy, —N═S(CH₃)₂═O, or have one of the following structures:

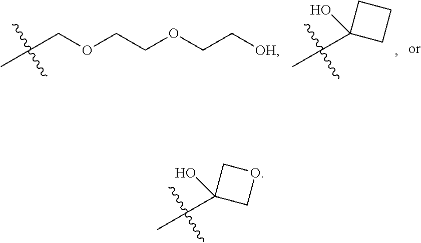 [SMILES: *COCCOCCO ; *C1(O)CCC1 ; "or" ; "," ; *C1(O)COC1]

In certain embodiments, R² has one of the following structures:

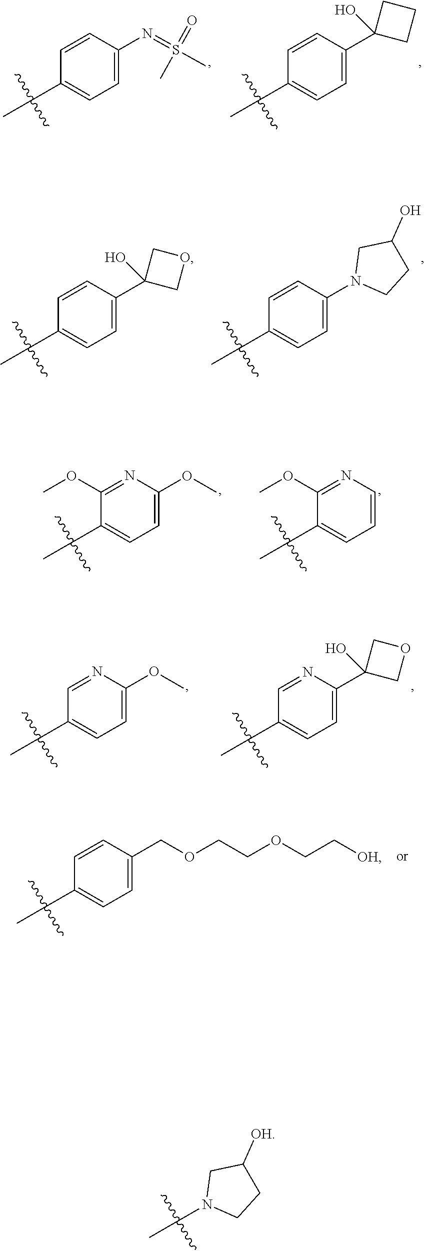

In some embodiments, R² has one of the following structures:

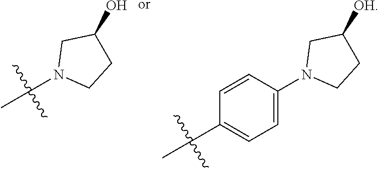 [SMILES: *N1CC[C@H](O)C1 ; "or" ; *c1ccc(N2CC[C@H](O)C2)cc1]

In certain embodiments, R² is —NH—R^{2d}. In some embodiments, R² has one of the following structures:

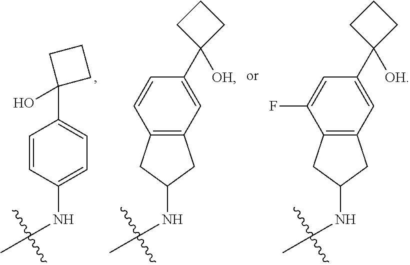 [SMILES: *Nc1ccc(C2(O)CCC2)cc1 ; "," ; *NC1Cc2ccc(C3(O)CCC3)cc2C1 ; "or" ; *NC1Cc2cc(C3(O)CCC3)cc(F)c2C1]

In some embodiments, R² has one of the following structures:

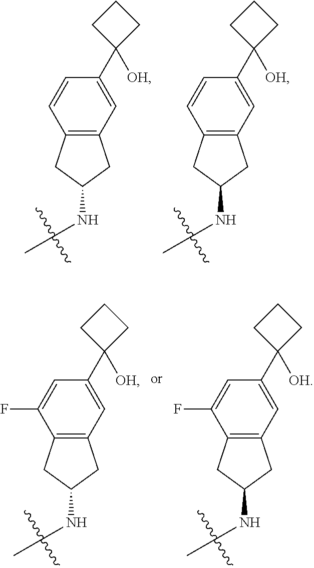 [SMILES: *N[C@@H]1Cc2ccc(C3(O)CCC3)cc2C1 ; *N[C@H]1Cc2ccc(C3(O)CCC3)cc2C1 ; *N[C@H]1Cc2cc(C3(O)CCC3)cc(F)c2C1 ; "or" ; *N[C@@H]1Cc2cc(C3(O)CCC3)cc(F)c2C1]

In certain embodiments, R³ has one of the following structures:

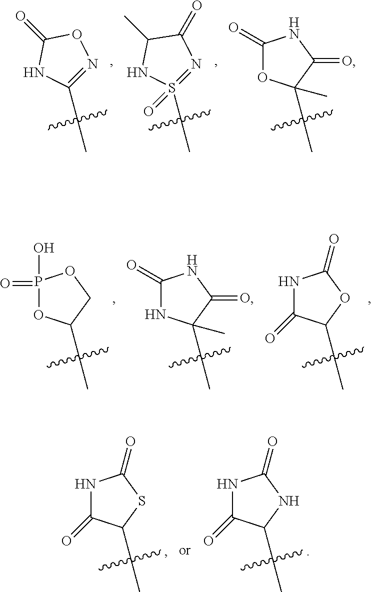

In some embodiments, R³ has one of the following structures:

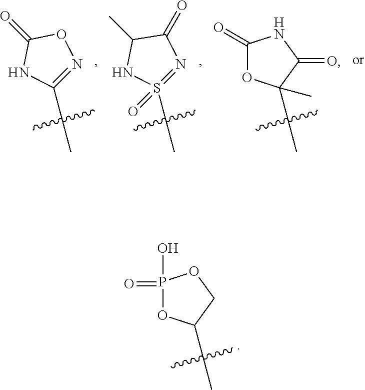

In some embodiments, R³ has one of the following structures:

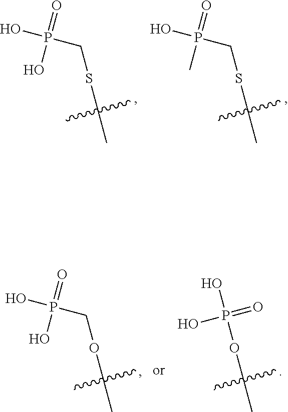

In certain embodiments, R³ has one of the following structures:

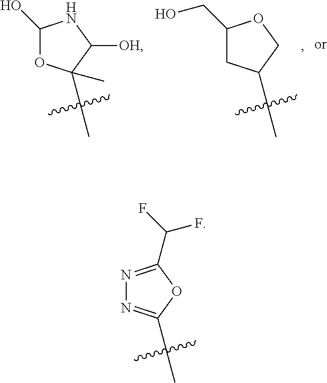

In certain embodiments, R³ has one of the following structures:

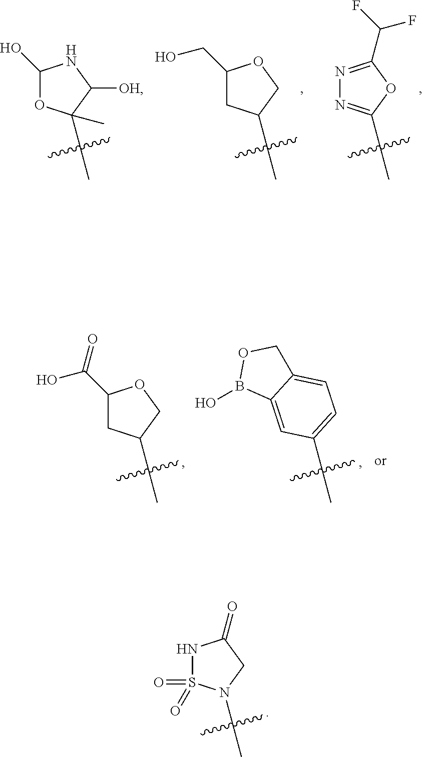

In some embodiments, R³ has one of the following structures:

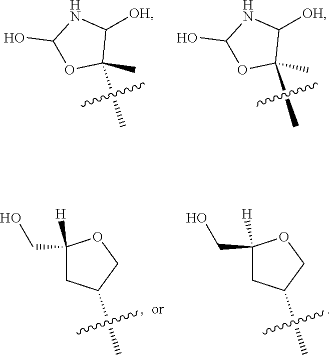

In various embodiments, the compound has one of the structures set forth in Table 1A below, or a pharmaceutically acceptable salt, stereoisomer, tautomer, solvate, polymorph, isotopologue, hydrate, or prodrug thereof. Compounds in Table 1A were prepared as described in the Examples or methods known in the art and analyzed by mass spectrometry and/or ¹H NMR.

TABLE 1A
Representative compounds of Structure (I)

| No. | Structure | Name |
|---|---|---|
| I-1 | 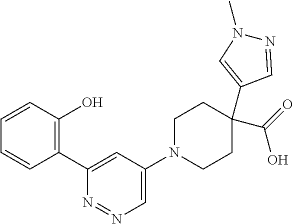 [SMILES: Cn1cc(C2(C(=O)O)CCN(c3cnnc(-c4ccccc4O)c3)CC2)cn1] | 3-(6-chloro-5-(4-(1- hydroxycyclobutyl) phenyl)- 1H-indol-3-yl)-1,2,4- oxadiazol-5(4H)-one |
| I-2 | 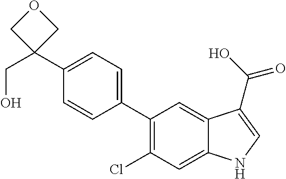 [SMILES: O=C(O)c1c[nH]c2cc(Cl)c(-c3ccc(C4(CO)COC4)cc3)cc12] | 1-(6-chloro-5-(4-(1- hydroxycyclobutyl) phenyl)- 1H-indol-3-yl)-4-methyl-4,5- dihydro-3H-1l6,2,5- thiadiazol-3-one 1-oxide (racemic) |
| I-3 | 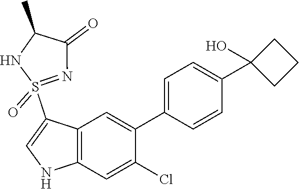 [SMILES: C[C@@H]1NS(=O)(c2c[nH]c3cc(Cl)c(-c4ccc(C5(O)CCC5)cc4)cc23)=NC1=O] | (4S)-1-(6-chloro-5-(4-(1- hydroxycyclobutyl)phenyl)- 1H-indol-3-yl)-4-methyl-4,5- dihydro-3H-1l6,2,5- thiadiazol-3-one 1-oxide |
| I-4 | 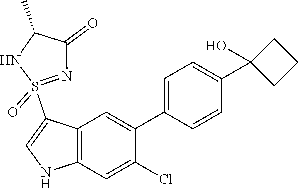 [SMILES: C[C@H]1NS(=O)(c2c[nH]c3cc(Cl)c(-c4ccc(C5(O)CCC5)cc4)cc23)=NC1=O] | (4R)-1-(6-chloro-5-(4-(1- hydroxycyclobutyl)phenyl)- 1H-indol-3-yl)-4-methyl-4,5- dihydro-3H-1l6,2,5- thiadiazol-3-one 1-oxide |
| I-5 | 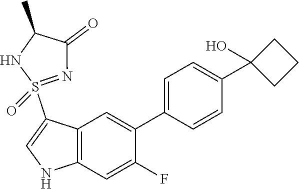 [SMILES: C[C@@H]1NS(=O)(c2c[nH]c3cc(F)c(-c4ccc(C5(O)CCC5)cc4)cc23)=NC1=O] | (4S)-1-(6-fluoro-5-(4-(1- hydroxycyclobutyl) phenyl)- 1H-indol-3-yl)-4-methyl-4,5- dihydro-3H-1l6,2,5- thiadiazol-3-one 1-oxide |
| I-6 | 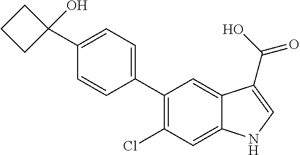 [SMILES: O=C(O)c1c[nH]c2cc(Cl)c(-c3ccc(C4(O)CCC4)cc3)cc12] | 3-(6-chloro-5-(4-(1- hydroxycyclobutyl) phenyl)- 1H-pyrrolo[2,3-b]pyridin-3- yl)-1,2,4-oxadiazol-5(4H)- one |
| I-7 | 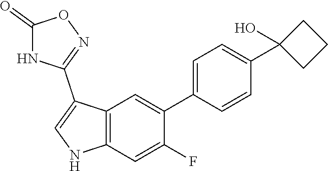 [SMILES: O=c1[nH]c(-c2c[nH]c3cc(F)c(-c4ccc(C5(O)CCC5)cc4)cc23)no1] | 3-(6-fluoro-5-(4-(1- hydroxycyclobutyl)phenyl)- 1H-indol-3-yl)-1,2,4- oxadiazol-5(4H)-one |
| I-8 | 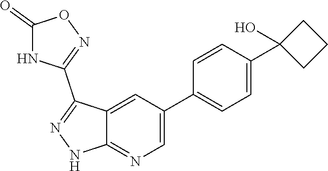 [SMILES: O=c1[nH]c(-c2n[nH]c3ncc(-c4ccc(C5(O)CCC5)cc4)cc23)no1] | 3-(5-(4-(1- hydroxycyclobutyl)phenyl)- 1H-pyrazolo[3,4-b]pyridin- 3-yl)-1,2,4-oxadiazol-5(4H)- one |
| I-9 | 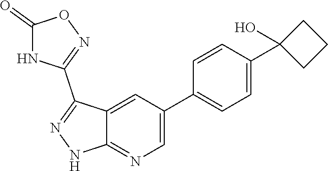 [SMILES: O=c1[nH]c(-c2n[nH]c3ncc(-c4ccc(C5(O)CCC5)cc4)cc23)no1] | 3-(5-(4-(1- hydroxycyclobutyl)phenyl)- 1H-pyrrolo[2,3-b]pyridin-3- yl)-1,2,4-oxadiazol-5(4H)- one |
| I-10 | 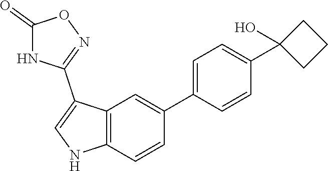 [SMILES: O=c1[nH]c(-c2c[nH]c3ccc(-c4ccc(C5(O)CCC5)cc4)cc23)no1] | 3-(5-(4-(1- hydroxycyclobutyl)phenyl)- 1H-indol-3-yl)-1,2,4- oxadiazol-5(4H)-one |
| I-11 | 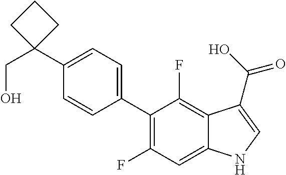 [SMILES: O=C(O)c1c[nH]c2cc(F)c(-c3ccc(C4(CO)CCC4)cc3)c(F)c12] | 3-(4,6-difluoro-5-(4-(1- hydroxycyclobutyl)phenyl)- 1H-indol-3-yl)-1,2,4- oxadiazol-5(4H)-one |
| I-12 | 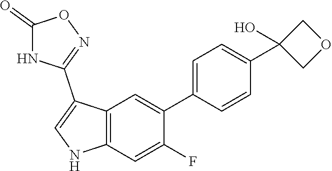 [SMILES: O=c1[nH]c(-c2c[nH]c3cc(F)c(-c4ccc(C5(O)COC5)cc4)cc23)no1] | 3-(6-fluoro-5-(4-(3- hydroxyoxetan-3-yl)phenyl)- 1H-indol-3-yl)-1,2,4- oxadiazol-5(4H)-one |
| I-13 | 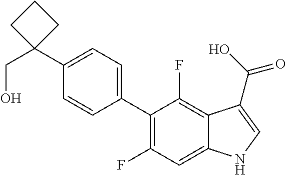 [SMILES: O=C(O)c1c[nH]c2cc(F)c(-c3ccc(C4(CO)CCC4)cc3)c(F)c12] | 3-(4,6-difluoro-5-(4-(3- hydroxyoxetan-3-yl)phenyl)- 1H-indol-3-yl)-1,2,4- oxadiazol-5(4H)-one |
| I-14 | 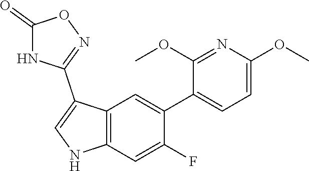 [SMILES: COc1ccc(-c2cc3c(-c4noc(=O)[nH]4)c[nH]c3cc2F)c(OC)n1] | 3-(5-(2,6-dimethoxypyridin- 3-yl)-6-fluoro-1H-indol-3- yl)-1,2,4-oxadiazol-5(4H)- one |
| I-15 | 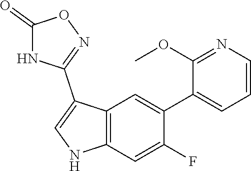 [SMILES: COc1ncccc1-c1cc2c(-c3noc(=O)[nH]3)c[nH]c2cc1F] | 3-(6-fluoro-5-(2- methoxypyridin-3-yl)-1H- indol-3-yl)-1,2,4-oxadiazol- 5(4H)-one |
| I-16 | 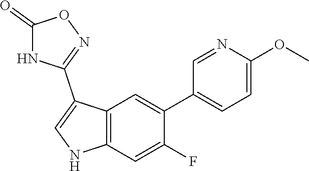 [SMILES: COc1ccc(-c2cc3c(-c4noc(=O)[nH]4)c[nH]c3cc2F)cn1] | 3-(6-fluoro-5-(6- methoxypyridin-3-yl)-1H- indol-3-yl)-1,2,4-oxadiazol- 5(4H)-one |
| I-17 | 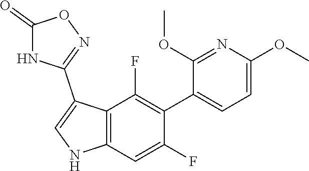 [SMILES: COc1ccc(-c2c(F)cc3[nH]cc(-c4noc(=O)[nH]4)c3c2F)c(OC)n1] | 3-(5-(2,6-dimethoxypyridin- 3-yl)-4,6-difluoro-1H-indol- 3-yl)-1,2,4-oxadiazol-5(4H)- one |
| I-18 | 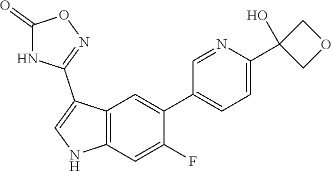 [SMILES: O=c1[nH]c(-c2c[nH]c3cc(F)c(-c4ccc(C5(O)COC5)nc4)cc23)no1] | 3-(6-fluoro-5-(6-(3- hydroxyoxetan-3-yl)pyridin- 3-yl)-1H-indol-3-yl)-1,2,4- oxadiazol-5(4H)-one |
| I-19 | 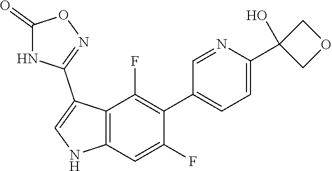 [SMILES: O=c1[nH]c(-c2c[nH]c3cc(F)c(-c4ccc(C5(O)COC5)nc4)c(F)c23)no1] | 3-(4,6-difluoro-5-(6-(3- hydroxyoxetan-3-yl)pyridin- 3-yl)-1H-indol-3-yl)-1,2,4- oxadiazol-5(4H)-one |
| I-20 | 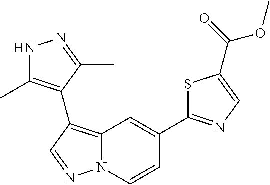 | (S)-3-(5-(3- hydroxypyrrolidin-1-yl)-1H- indol-3-yl)-1,2,4-oxadiazol- 5(4H)-one |
| I-21 | 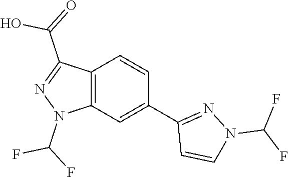 [SMILES: O=C(O)c1nn(C(F)F)c2cc(-c3ccn(C(F)F)n3)ccc12] | (S)-3-(6-fluoro-5-(3- hydroxypyrrolidin-1-yl)-1H- indol-3-yl)-1,2,4-oxadiazol- 5(4H)-one |
| I-22 | 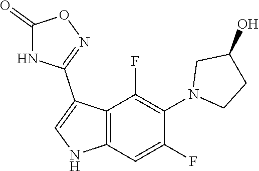 [SMILES: O=c1[nH]c(-c2c[nH]c3cc(F)c(N4CC[C@H](O)C4)c(F)c23)no1] | (S)-3-(4,6-difluoro-5-(3- hydroxypyrrolidin-1-yl)-1H- indol-3-yl)-1,2,4-oxadiazol- 5(4H)-one |
| I-23 | 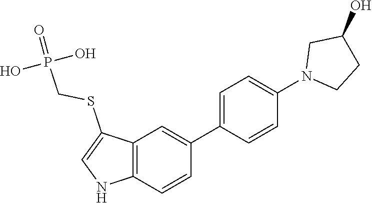 [SMILES: O=P(O)(O)CSc1c[nH]c2ccc(-c3ccc(N4CC[C@H](O)C4)cc3)cc12] | (S)-(((5-(4-(3- hydroxypyrrolidin-1- yl)phenyl)-1H-indol-3- yl)thio)methyl)phosphonic acid |
| I-24 | 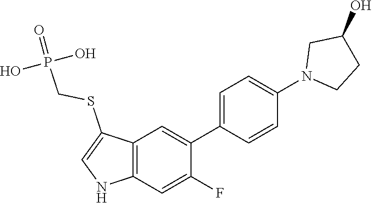 [SMILES: O=P(O)(O)CSc1c[nH]c2cc(F)c(-c3ccc(N4CC[C@H](O)C4)cc3)cc12] | (S)-(((6-fluoro-5-(4-(3- hydroxypyrrolidin-1- yl)phenyl)-1H-indol-3- yl)thio)methyl)phosphonic acid |
| I-25 | 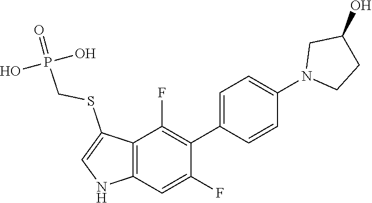 [SMILES: O=P(O)(O)CSc1c[nH]c2cc(F)c(-c3ccc(N4CC[C@H](O)C4)cc3)c(F)c12] | (S)-(((4,6-difluoro-5-(4-(3- hydroxypyrrolidin-1- yl)phenyl)-1H-indol-3- yl)thio)methyl)phosphonic acid |
| I-26 | 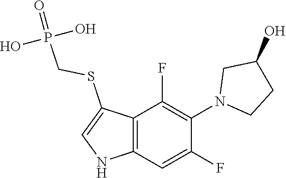 [SMILES: O=P(O)(O)CSc1c[nH]c2cc(F)c(N3CC[C@H](O)C3)c(F)c12] | (S)-(((4,6-difluoro-5-(3- hydroxypyrrolidin-1-yl)-1H- indol-3-yl)thio)methyl) phosphonic acid |
| I-27 | 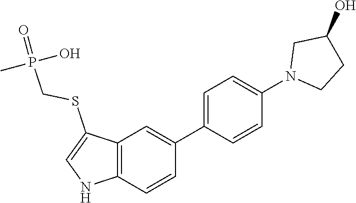 [SMILES: CP(=O)(O)CSc1c[nH]c2ccc(-c3ccc(N4CC[C@H](O)C4)cc3)cc12] | (((5-(4-((S)-3- hydroxypyrrolidin-1- yl)phenyl)-1H-indol-3- yl)thio)methyl)(methyl) phosphinic acid |
| I-28 | 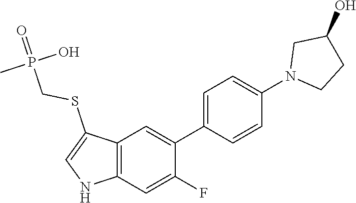 [SMILES: CP(=O)(O)CSc1c[nH]c2cc(F)c(-c3ccc(N4CC[C@H](O)C4)cc3)cc12] | (((6-fluoro-5-(4-((S)-3- hydroxypyrrolidin-1- yl)phenyl)-1H-indol-3- yl)thio)methyl)(methyl) phosphinic acid |
| I-29 | 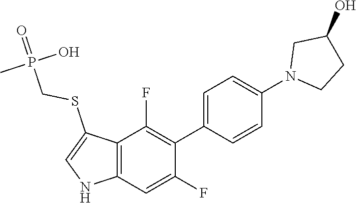 [SMILES: CP(=O)(O)CSc1c[nH]c2cc(F)c(-c3ccc(N4CC[C@H](O)C4)cc3)c(F)c12] | (((4,6-difluoro-5-(4-((S)-3- hydroxypyrrolidin-1- yl)phenyl)-1H-indol-3- yl)thio)methyl)(methyl) phosphinic acid |
| I-30 | 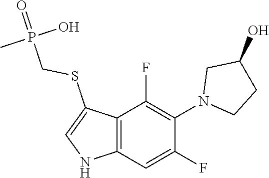 [SMILES: CP(=O)(O)CSc1c[nH]c2cc(F)c(N3CC[C@H](O)C3)c(F)c12] | (((4,6-difluoro-5-((S)-3- hydroxypyrrolidin-1-yl)-1H- indol-3- yl)thio)methyl)(methyl) phosphinic acid |
| I-31 | 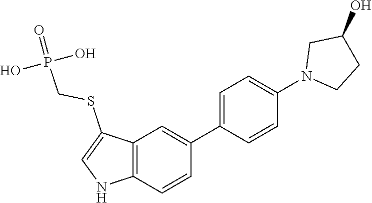 [SMILES: O=P(O)(O)CSc1c[nH]c2ccc(-c3ccc(N4CC[C@H](O)C4)cc3)cc12] | (S)-(((5-(4-(3- hydroxypyrrolidin-1- yl)phenyl)-1H-indol-3- yl)oxy)methyl)phosphonic acid |
| I-32 | 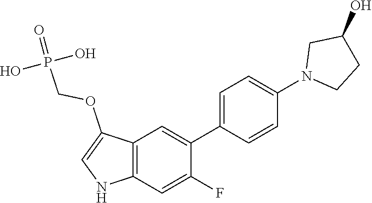 [SMILES: O=P(O)(O)COc1c[nH]c2cc(F)c(-c3ccc(N4CC[C@H](O)C4)cc3)cc12] | (S)-(((6-fluoro-5-(4-(3- hydroxypyrrolidin-1- yl)phenyl)-1H-indol-3- yl)oxy)methyl)phosphonic acid |
| I-33 | 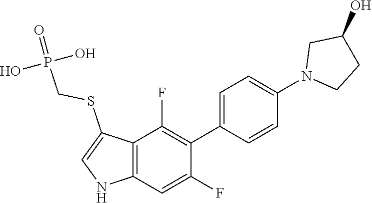 [SMILES: O=P(O)(O)CSc1c[nH]c2cc(F)c(-c3ccc(N4CC[C@H](O)C4)cc3)c(F)c12] | (S)-(((4,6-difluoro-5-(4-(3- hydroxypyrrolidin-1- yl)phenyl)-1H-indol-3- yl)oxy)methyl)phosphonic acid |
| I-34 | 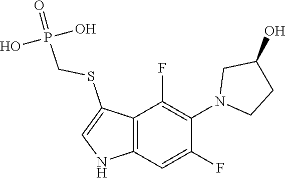 [SMILES: O=P(O)(O)CSc1c[nH]c2cc(F)c(N3CC[C@H](O)C3)c(F)c12] | (S)-(((4,6-difluoro-5-(3- hydroxypyrrolidin-1-yl)-1H- indol-3-yl)oxy)methyl) phosphonic acid |
| I-35 | 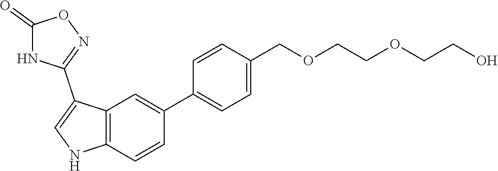 [SMILES: O=c1[nH]c(-c2c[nH]c3ccc(-c4ccc(COCCOCCO)cc4)cc23)no1] | 3-(5-(4-((2-(2- hydroxyethoxy)ethoxy) methyl)phenyl)-1H-indol-3- yl)-1,2,4-oxadiazol-5(4H)- one |
| I-36 | 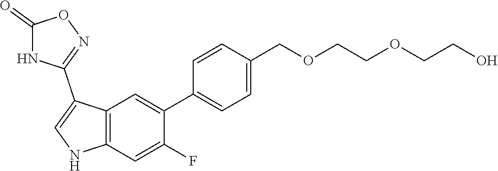 [SMILES: O=c1[nH]c(-c2c[nH]c3cc(F)c(-c4ccc(COCCOCCO)cc4)cc23)no1] | 3-(6-fluoro-5-(4-((2-(2- hydroxyethoxy)ethoxy) methyl)phenyl)-1H-indol-3- yl)-1,2,4-oxadiazol-5(4H)- one |
| I-37 | 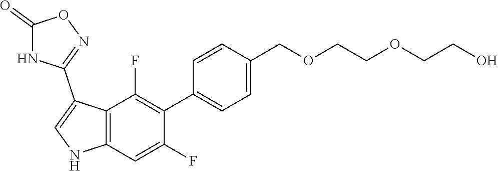 [SMILES: O=c1[nH]c(-c2c[nH]c3cc(F)c(-c4ccc(COCCOCCO)cc4)c(F)c23)no1] | 3-(4,6-difluoro-5-(4-((2-(2- hydroxyethoxy)ethoxy) methyl)phenyl)-1H-indol-3- yl)-1,2,4-oxadiazol-5(4H)- one |
| I-38 | 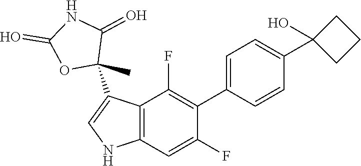 [SMILES: C[C@@]1(c2c[nH]c3cc(F)c(-c4ccc(C5(O)CCC5)cc4)c(F)c23)OC(=O)NC1=O] | (S)-5-(4,6-difluoro-5-(4-(1- hydroxycyclobutyl)phenyl)- 1H-indol-3-yl)-5- methyloxazolidine-2,4-dione |
| I-39 | 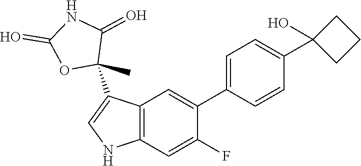 [SMILES: C[C@@]1(c2c[nH]c3cc(F)c(-c4ccc(C5(O)CCC5)cc4)cc23)OC(=O)NC1=O] | (S)-5-(6-fluoro-5-(4-(1- hydroxycyclobutyl)phenyl)- 1H-indol-3-yl)-5- methyloxazolidine-2,4-dione |
| I-40 | 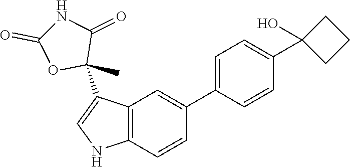 [SMILES: C[C@@]1(c2c[nH]c3ccc(-c4ccc(C5(O)CCC5)cc4)cc23)OC(=O)NC1=O] | (S)-5-(5-(4-(1- hydroxycyclobutyl)phenyl)- 1H-indol-3-yl)-5- methyloxazolidine-2,4-dione |
| I-41 | 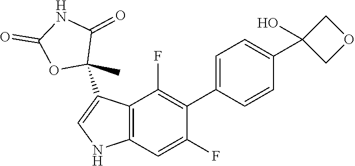 [SMILES: C[C@@]1(c2c[nH]c3cc(F)c(-c4ccc(C5(O)COC5)cc4)c(F)c23)OC(=O)NC1=O] | (S)-5-(4,6-difluoro-5-(4-(3- hydroxyoxetan-3-yl)phenyl)- 1H-indol-3-yl)-5- methyloxazolidine-2,4-dione |
| I-42 | 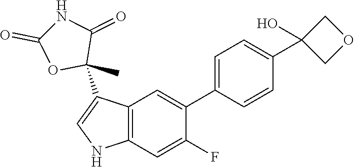 [SMILES: C[C@@]1(c2c[nH]c3cc(F)c(-c4ccc(C5(O)COC5)cc4)cc23)OC(=O)NC1=O] | (S)-5-(6-fluoro-5-(4-(3- hydroxyoxetan-3-yl)phenyl)- 1H-indol-3-yl)-5- methyloxazolidine-2,4-dione |
| I-43 | 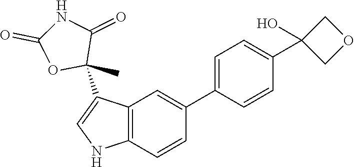 [SMILES: C[C@@]1(c2c[nH]c3ccc(-c4ccc(C5(O)COC5)cc4)cc23)OC(=O)NC1=O] | (S)-5-(5-(4-(3- hydroxyoxetan-3-yl)phenyl)- 1H-indol-3-yl)-5- methyloxazolidine-2,4-dione |
| I-44 | 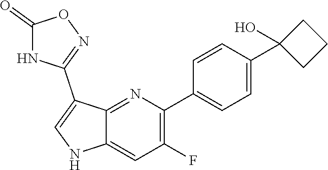 [SMILES: O=c1[nH]c(-c2c[nH]c3cc(F)c(-c4ccc(C5(O)CCC5)cc4)nc23)no1] | 3-(6-fluoro-5-(4-(1- hydroxycyclobutyl)phenyl)- 1H-pyrrolo[3,2-b]pyridin-3- yl)-1,2,4-oxadiazol-5(4H)- one |
| I-45 | 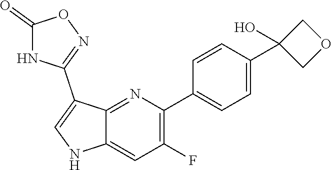 [SMILES: O=c1[nH]c(-c2c[nH]c3cc(F)c(-c4ccc(C5(O)COC5)cc4)nc23)no1] | 3-(6-fluoro-5-(4-(3- hydroxyoxetan-3-yl)phenyl)- 1H-pyrrolo[3,2-b]pyridin-3- yl)-1,2,4-oxadiazol-5(4H)- one |
| I-46 | 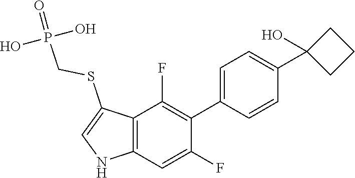 [SMILES: O=P(O)(O)CSc1c[nH]c2cc(F)c(-c3ccc(C4(O)CCC4)cc3)c(F)c12] | (((4,6-difluoro-5-(4-(1- hydroxycyclobutyl)phenyl)- 1H-indol-3-yl)thio)methyl) phosphonic acid |
| I-47 | 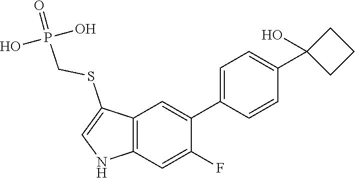 [SMILES: O=P(O)(O)CSc1c[nH]c2cc(F)c(-c3ccc(C4(O)CCC4)cc3)cc12] | (((6-fluoro-5-(4-(1- hydroxycyclobutyl)phenyl)- 1H-indol-3-yl)thio)methyl) phosphonic acid |
| I-48 | 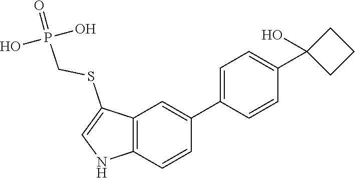 [SMILES: O=P(O)(O)CSc1c[nH]c2ccc(-c3ccc(C4(O)CCC4)cc3)cc12] | (((5-(4-(1-hydroxycyclobutyl) phenyl)-1H-indol-3-yl)thio) methyl)phosphonic acid |
| I-49 | 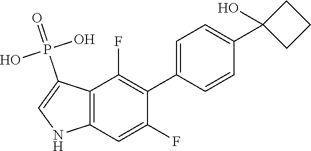 [SMILES: O=P(O)(O)c1c[nH]c2cc(F)c(-c3ccc(C4(O)CCC4)cc3)c(F)c12] | (4,6-difluoro-5-(4-(1- hydroxycyclobutyl)phenyl)- 1H-indol-3-yl)phosphonic acid |
| I-50 | 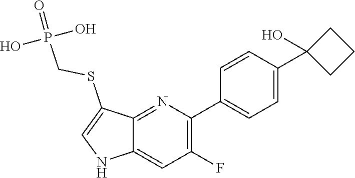 [SMILES: O=P(O)(O)CSc1c[nH]c2cc(F)c(-c3ccc(C4(O)CCC4)cc3)nc12] | (((6-fluoro-5-(4-(1- hydroxycyclobutyl)phenyl)- 1H-pyrrolo[3,2-b]pyridin-3- yl)thio)methyl)phosphonic acid |
| I-51 | 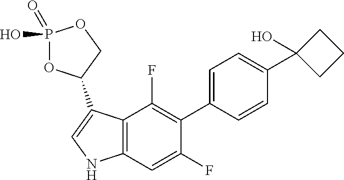 [SMILES: O=[P@]1(O)OC[C@H](c2c[nH]c3cc(F)c(-c4ccc(C5(O)CCC5)cc4)c(F)c23)O1] | (2S,4S)-4-(4,6-difluoro-5-(4- (1-hydroxycyclobutyl) phenyl)-1H-indol-3-yl)-2- hydroxy-1,3,2- dioxaphospholane 2-oxide |
| I-52 | 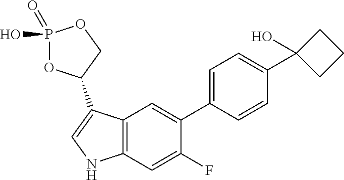 [SMILES: O=[P@]1(O)OC[C@H](c2c[nH]c3cc(F)c(-c4ccc(C5(O)CCC5)cc4)cc23)O1] | (2S,4S)-4-(6-fluoro-5-(4-(1- hydroxycyclobutyl)phenyl)- 1H-indol-3-yl)-2-hydroxy- 1,3,2-dioxaphospholane 2- oxide |
| I-53 | 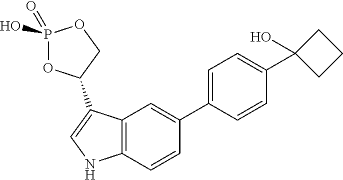 [SMILES: O=[P@]1(O)OC[C@H](c2c[nH]c3ccc(-c4ccc(C5(O)CCC5)cc4)cc23)O1] | (2S,4S)-2-hydroxy-4-(5-(4- (1-hydroxycyclobutyl) phenyl)-1H-indol-3-yl)-1,3,2- dioxaphospholane 2-oxide |
| I-54 | 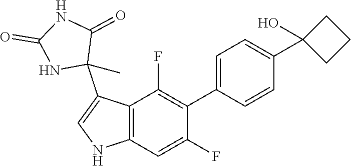 [SMILES: CC1(c2c[nH]c3cc(F)c(-c4ccc(C5(O)CCC5)cc4)c(F)c23)NC(=O)NC1=O] | 5-(4,6-difluoro-5-(4-(1- hydroxycyclobutyl)phenyl)- 1H-indol-3-yl)-5- methylimidazolidine-2,4- dione |
| I-55 | 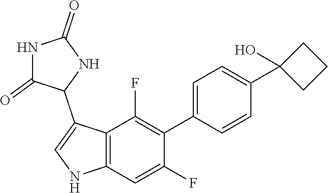 [SMILES: O=C1NC(=O)C(c2c[nH]c3cc(F)c(-c4ccc(C5(O)CCC5)cc4)c(F)c23)N1] | 5-(4,6-difluoro-5-(4-(1- hydroxycyclobutyl)phenyl)- 1H-indol-3-yl)imidazolidine- 2,4-dione |
| I-56 | 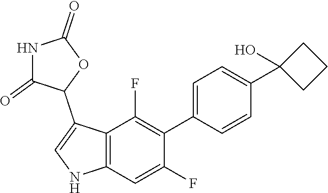 [SMILES: O=C1NC(=O)C(c2c[nH]c3cc(F)c(-c4ccc(C5(O)CCC5)cc4)c(F)c23)O1] | 5-(4,6-difluoro-5-(4-(1- hydroxycyclobutyl)phenyl)- 1H-indol-3-yl)oxazolidine- 2,4-dione |
| I-57 | 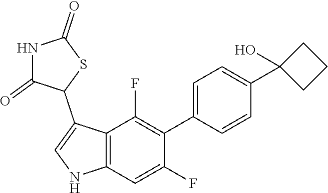 [SMILES: O=C1NC(=O)C(c2c[nH]c3cc(F)c(-c4ccc(C5(O)CCC5)cc4)c(F)c23)S1] | 5-(4,6-difluoro-5-(4-(1- hydroxycyclobutyl)phenyl)- 1H-indol-3-yl)thiazolidine- 2,4-dione |
| I-58 | 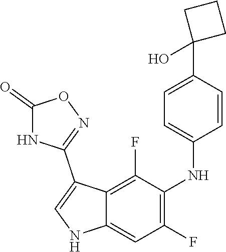 [SMILES: O=c1[nH]c(-c2c[nH]c3cc(F)c(Nc4ccc(C5(O)CCC5)cc4)c(F)c23)no1] | 3-(4,6-difluoro-5-((4-(1- hydroxycyclobutyl)phenyl) amino)-1H-indol-3-yl)-1,2,4- oxadiazol-5(4H)-one |
| I-59 | 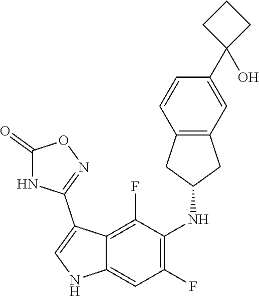 [SMILES: O=c1[nH]c(-c2c[nH]c3cc(F)c(N[C@@H]4Cc5ccc(C6(O)CCC6)cc5C4)c(F)c23)no1] | (R)-3-(4,6-difluoro-5-((5-(1- hydroxycyclobutyl)-2,3- dihydro-1H-inden-2- yl)amino)-1H-indol-3-yl)- 1,2,4-oxadiazol-5(4H)-one |
| I-60 | 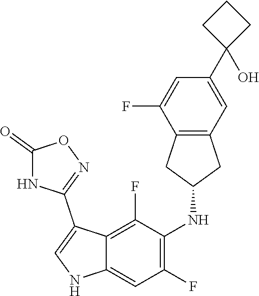 [SMILES: O=c1[nH]c(-c2c[nH]c3cc(F)c(N[C@H]4Cc5cc(C6(O)CCC6)cc(F)c5C4)c(F)c23)no1] | (S)-3-(4,6-difluoro-5-((4- fluoro-6-(1- hydroxycyclobutyl)-2,3- dihydro-1H-inden-2- yl)amino)-1H-indol-3-yl)- 1,2,4-oxadiazol-5(4H)-one |
| I-61 | 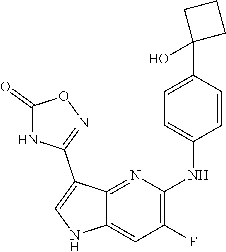 [SMILES: O=c1[nH]c(-c2c[nH]c3cc(F)c(Nc4ccc(C5(O)CCC5)cc4)nc23)no1] | 3-(6-fluoro-5-((4-(1- hydroxycyclobutyl)phenyl) amino)-1H-pyrrolo[3,2- b]pyridin-3-yl)-1,2,4- oxadiazol-5(4H)-one |
| I-62 | 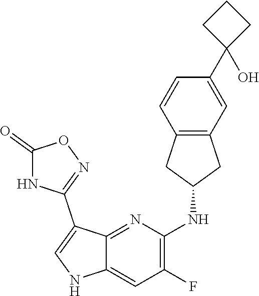 [SMILES: O=c1[nH]c(-c2c[nH]c3cc(F)c(N[C@@H]4Cc5ccc(C6(O)CCC6)cc5C4)nc23)no1] | (R)-3-(6-fluoro-5-((5-(1- hydroxycyclobutyl)-2,3- dihydro-1H-inden-2- yl)amino)-1H-pyrrolo[3,2- b]pyridin-3-yl)-1,2,4- oxadiazol-5(4H)-one |
| I-63 | 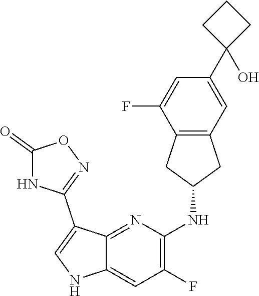 [SMILES: O=c1[nH]c(-c2c[nH]c3cc(F)c(N[C@H]4Cc5cc(C6(O)CCC6)cc(F)c5C4)nc23)no1] | (S)-3-(6-fluoro-5-((4-fluoro- 6-(1-hydroxycyclobutyl)-2,3- dihydro-1H-inden-2- yl)amino)-1H-pyrrolo[3,2- b]pyridin-3-yl)-1,2,4- oxadiazol-5(4H)-one |
| I-64 | 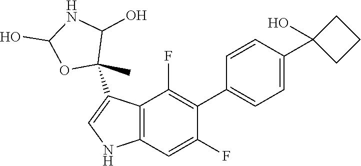 [SMILES: C[C@@]1(c2c[nH]c3cc(F)c(-c4ccc(C5(O)CCC5)cc4)c(F)c23)OC(O)NC1O] | (5S)-5-(4,6-difluoro-5-(4-(1- hydroxycyclobutyl)phenyl)- 1H-indol-3-yl)-5- methyloxazolidine-2,4-diol |
| I-65 | 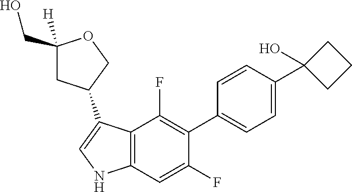 [SMILES: OC[C@@H]1C[C@@H](c2c[nH]c3cc(F)c(-c4ccc(C5(O)CCC5)cc4)c(F)c23)CO1] | 1-(4-(4,6-difluoro-3-((3S,5S)- 5- (hydroxymethyl) tetrahydrofuran- 3-yl)-1H-indol-5- yl)phenyl)cyclobutan-1-ol |
| I-66 | 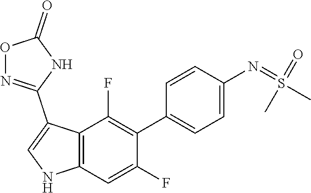 [SMILES: CS(C)(=O)=Nc1ccc(-c2c(F)cc3[nH]cc(-c4noc(=O)[nH]4)c3c2F)cc1] | 3-(5-(4-((dimethyl(oxo)-16- sulfaneylidene)amino)phenyl)- 4,6-difluoro-1H-indol-3-yl)- 1,2,4-oxadiazol-5(4H)-one |
| I-67 | 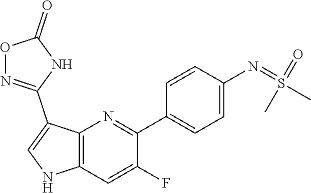 [SMILES: CS(C)(=O)=Nc1ccc(-c2nc3c(-c4noc(=O)[nH]4)c[nH]c3cc2F)cc1] | 3-(5-(4-((dimethyl(oxo)-l6- sulfaneylidene)amino)phenyl)- 6-fluoro-1H-pyrrolo[3,2- b]pyridin-3-yl)-1,2,4- oxadiazol-5(4H)-one |
| I-68 | 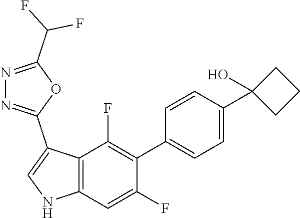 [SMILES: OC1(c2ccc(-c3c(F)cc4[nH]cc(-c5nnc(C(F)F)o5)c4c3F)cc2)CCC1] | 1-(4-(3-(5-(difluoromethyl)- 1,3,4-oxadiazol-2-yl)-4,6- difluoro-1H-indol-5- yl)phenyl)cyclobutan-1-ol |
| I-69 | 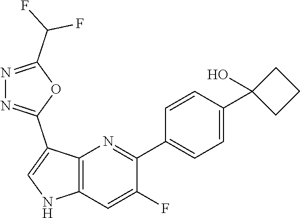 [SMILES: OC1(c2ccc(-c3nc4c(-c5nnc(C(F)F)o5)c[nH]c4cc3F)cc2)CCC1] | 1-(4-(3-(5-(difluoromethyl)- 1,3,4-oxadiazol-2-yl)-6- fluoro-1H-pyrrolo[3,2- b]pyridin-5- yl)phenyl)cyclobutan-1-ol |
| I-70 | 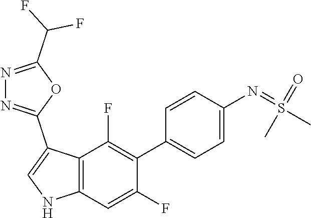 [SMILES: CS(C)(=O)=Nc1ccc(-c2c(F)cc3[nH]cc(-c4nnc(C(F)F)o4)c3c2F)cc1] | ((4-(3-(5-(difluoromethyl)- 1,3,4-oxadiazol-2-yl)-4,6- difluoro-1H-indol-5- yl)phenyl)imino)dimethyl-l6- sulfanone |
| I-71 | 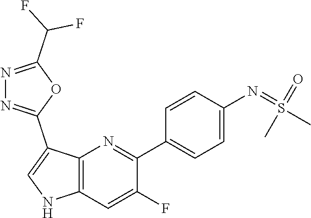 [SMILES: CS(C)(=O)=Nc1ccc(-c2nc3c(-c4nnc(C(F)F)o4)c[nH]c3cc2F)cc1] | ((4-(3-(5-(difluoromethyl)- 1,3,4-oxadiazol-2-yl)-6- fluoro-1H-pyrrolo[3,2- b]pyridin-5- yl)phenyl)imino)dimethyl-l6- sulfanone |
| I-72 | 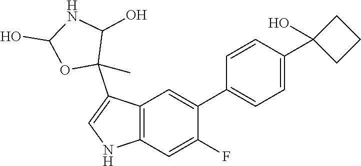 [SMILES: CC1(c2c[nH]c3cc(F)c(-c4ccc(C5(O)CCC5)cc4)cc23)OC(O)NC1O] | 5-(6-fluoro-5-(4-(1- hydroxycyclobutyl)phenyl)- 1H-indol-3-yl)-5- methyloxazolidine-2,4-diol |
| I-73 | 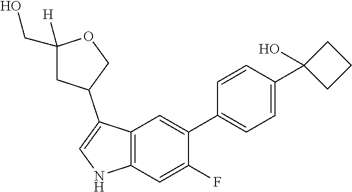 [SMILES: OCC1CC(c2c[nH]c3cc(F)c(-c4ccc(C5(O)CCC5)cc4)cc23)CO1] | 1-(4-(6-fluoro-3-(5- (hydroxymethyl) tetrahydrofuran- 3-yl)-1H-indol-5- yl)phenyl)cyclobutan-1-ol |
| I-74 | 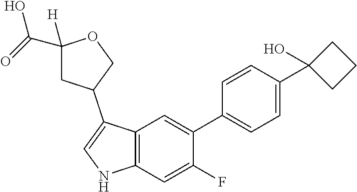 [SMILES: O=C(O)C1CC(c2c[nH]c3cc(F)c(-c4ccc(C5(O)CCC5)cc4)cc23)CO1] | 4-(6-fluoro-5-(4-(1- hydroxycyclobutyl)phenyl)- 1H-indol-3- yl)tetrahydrofuran-2- carboxylic acid |
| I-75 | 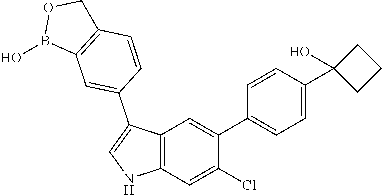 [SMILES: OB1OCc2ccc(-c3c[nH]c4cc(Cl)c(-c5ccc(C6(O)CCC6)cc5)cc34)cc21] | 6-(6-chloro-5-(4-(1- hydroxycyclobutyl)phenyl)- 1H-indol-3- yl)benzo[c][1,2]oxaborol- 1(3H)-ol |
| I-76 | 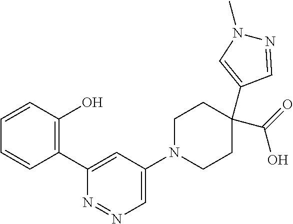 [SMILES: Cn1cc(C2(C(=O)O)CCN(c3cnnc(-c4ccccc4O)c3)CC2)cn1] | 5-(6-chloro-5-(4-(1- hydroxycyclobutyl)phenyl)- 1H-indol-3-yl)-1,2,5- thiadiazolidin-3-one 1,1- dioxide |

One embodiment provides a compound of having the following Structure (II):

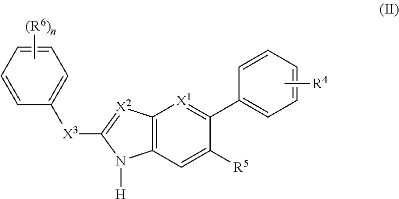

or a pharmaceutically acceptable salt, stereoisomer, or tautomer thereof, wherein:
X¹ is N or CR⁷;
X² is N or CH;
X³ is —NH—, —O—, or —S—;
R⁴ is optionally substituted C₃-C₆ cycloalkyl or —N═S(R^{4a})₂═O, wherein R^{4a} is, at each occurrence, independently C₁-C₄ alkyl;
R⁵ is hydrogen or halo;
R⁶ is, at each occurrence, independently optionally substituted C₁-C₄ alkyl, optionally substituted 5-membered heterocyclyl;
R⁷ is hydrogen or halo; and
n is 0, 1, or 2.

One embodiment provides a compound of having the following Structure (II):

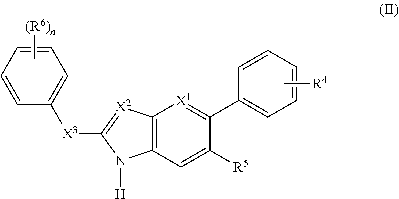

or a pharmaceutically acceptable salt, stereoisomer, or tautomer thereof, wherein:
X¹ is N or CR⁷;
X² is N or CH;
X³ is —NH—, —O—, or —S—;
R⁴ is optionally substituted C₃-C₆ cycloalkyl, —N═S(R^{4a})₂═O, or optionally substituted phenyl, wherein R^{4a} is, at each occurrence, independently C₁-C₄ alkyl;
R⁵ is hydrogen or halo;
R⁶ is, at each occurrence, independently optionally substituted C₁-C₄ alkyl, optionally substituted 5-membered heterocyclyl, or
two occurrences of R⁶ join, together with the carbons to which they are attached to form a 4-6 membered heterocyclyl;
R⁷ is hydrogen or halo; and
n is 0, 1, 2, or 3.

In some embodiments, X¹ is N. In certain embodiments, X¹ is CF. In some embodiments, X¹ is N. In certain embodiments, X¹ is CF of CH. In some embodiments, X² is N. In certain embodiments, X² is CH. In some embodiments, X³ is —NH—. In certain embodiments, X³ is —O—. In certain embodiments, X³ is —S—.

In some embodiments, R⁴ is C₃-C₆ cycloalkyl optionally substituted with —OH. In certain embodiments, R⁴ has the following structure:

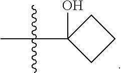 [SMILES: *C1(O)CCC1]

In certain embodiments, R⁴ is —N═S(R^{4a})₂═O and both occurrences of R^{4a} are —CH₃. In some embodiments, R⁴ is unsubstituted phenyl.

In some embodiments, R⁵ is fluoro. In some embodiments, R⁵ is fluoro or chloro.

In certain embodiments, one occurrence of R⁶ is —CH₃. In some embodiments, one occurrence of R⁶ is 5-membered heterocyclyl optionally substituted with oxo. In certain embodiments, one occurrence of R⁶ has the following structure:

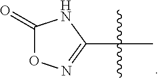 [SMILES: *c1noc(=O)[nH]1]

In certain embodiments, one occurrence of Re has one of the following structures:

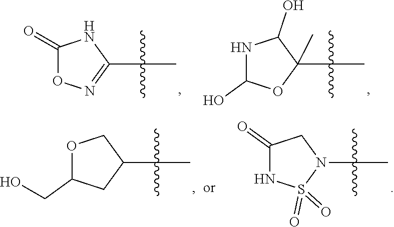

In some embodiments,

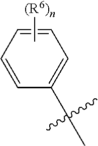

has one of the following structures:

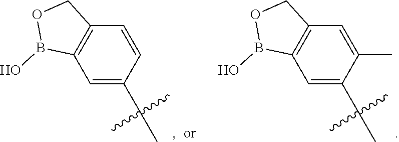 [SMILES: *c1ccc2c(c1)B(O)OC2 ; *c1cc2c(cc1C)COB2O]

In certain embodiments, n is 1. In some embodiments, n is 2.

In various embodiments, the compound has one of the structures set forth in Table 1B below, or a pharmaceutically acceptable salt, stereoisomer, tautomer, solvate, polymorph, isotopologue, hydrate, or prodrug thereof. Compounds in Table 1B were prepared as described in the Examples or methods known in the art and analyzed by mass spectrometry and/or ¹H NMR.

TABLE 1B
Representative compounds of Structure (II)

| No. | Structure | Name |
|---|---|---|
| II-1 | 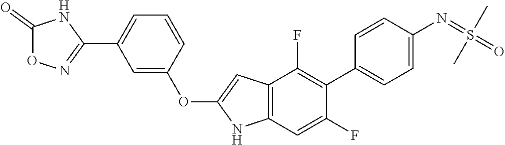 [SMILES: CS(C)(=O)=Nc1ccc(-c2c(F)cc3[nH]c(Oc4cccc(-c5noc(=O)[nH]5)c4)cc3c2F)cc1] | 3-(3-((5-(4-((dimethyl(oxo)-l6- sulfaneylidene)amino)phenyl)- 4,6-difluoro-1H-indol-2- yl)oxy)phenyl)-1,2,4-oxadiazol- 5(4H)-one |
| II-2 | 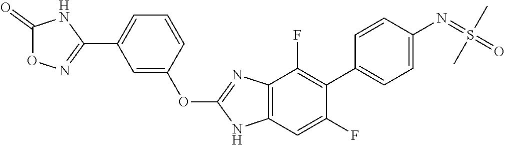 [SMILES: CS(C)(=O)=Nc1ccc(-c2c(F)cc3[nH]c(Oc4cccc(-c5noc(=O)[nH]5)c4)nc3c2F)cc1] | 3-(3-((5-(4-((dimethyl(oxo)-λ⁶- sulfaneylidene)amino)phenyl)- 4,6-difluoro-1H- benzo[d]imidazol-2- yl)oxy)phenyl)-1,2,4-oxadiazol- 5(4H)-one |
| II-3 | 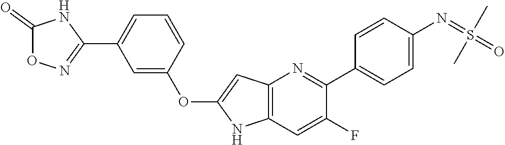 [SMILES: CS(C)(=O)=Nc1ccc(-c2nc3cc(Oc4cccc(-c5noc(=O)[nH]5)c4)[nH]c3cc2F)cc1] | 3-(3-((5-(4-((dimethyl(oxo)-l6- sulfaneylidene)amino)phenyl)- 4,6-difluoro-1H- benzo[d]imidazol-2- yl)oxy)phenyl)-1,2,4-oxadiazol- 5(4H)-one |
| II-4 | 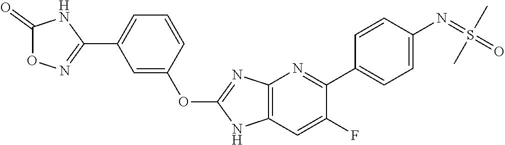 [SMILES: CS(C)(=O)=Nc1ccc(-c2nc3nc(Oc4cccc(-c5noc(=O)[nH]5)c4)[nH]c3cc2F)cc1] | 3-(3-((5-(4-((dimethyl(oxo)-l6- sulfaneylidene)amino)phenyl)-6- fluoro-1H-imidazo[4,5- b]pyridin-2-yl)oxy)phenyl)- 1,2,4-oxadiazol-5(4H)-one |
| II-5 | 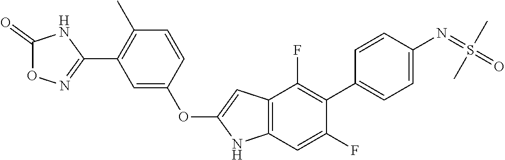 [SMILES: Cc1ccc(Oc2cc3c(F)c(-c4ccc(N=S(C)(C)=O)cc4)c(F)cc3[nH]2)cc1-c1noc(=O)[nH]1] | 3-(5-((5-(4-((dimethyl(oxo)-l6- sulfaneylidene)amino)phenyl)- 4,6-difluoro-1H-indol-2-yl)oxy)- 2-methylphenyl)-1,2,4- oxadiazol-5(4H)-one |
| II-6 | 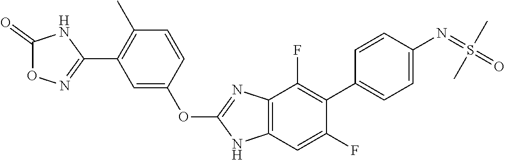 [SMILES: Cc1ccc(Oc2nc3c(F)c(-c4ccc(N=S(C)(C)=O)cc4)c(F)cc3[nH]2)cc1-c1noc(=O)[nH]1] | 3-(5-((5-(4-((dimethyl(oxo)-l6- sulfaneylidene)amino)phenyl)- 4,6-difluoro-1H- benzo[d]imidazol-2-yl)oxy)-2- methylphenyl)-1,2,4-oxadiazol- 5(4H)-one |
| II-7 | 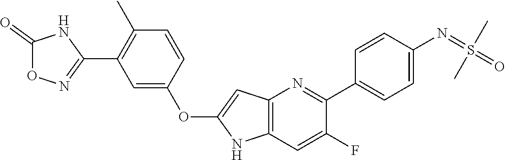 [SMILES: Cc1ccc(Oc2cc3nc(-c4ccc(N=S(C)(C)=O)cc4)c(F)cc3[nH]2)cc1-c1noc(=O)[nH]1] | 3-(5-((5-(4-((dimethyl(oxo)-l6- sulfaneylidene)amino)phenyl)-6- fluoro-1H-pyrrolo[3,2-b]pyridin- 2-yl)oxy)-2-methylphenyl)- 1,2,4-oxadiazol-5(4H)-one |
| II-8 | 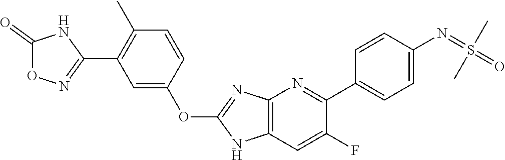 [SMILES: Cc1ccc(Oc2nc3nc(-c4ccc(N=S(C)(C)=O)cc4)c(F)cc3[nH]2)cc1-c1noc(=O)[nH]1] | 3-(5-((5-(4-((dimethyl(oxo)-l6- sulfaneylidene)amino)phenyl)-6- fluoro-1H-imidazo[4,5- b]pyridin-2-yl)oxy)-2- methylphenyl)-1,2,4-oxadiazol- 5(4H)-one |
| II-9 | 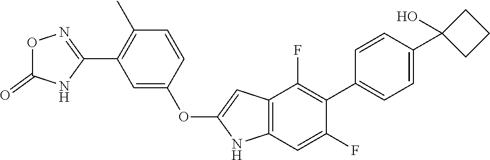 [SMILES: Cc1ccc(Oc2cc3c(F)c(-c4ccc(C5(O)CCC5)cc4)c(F)cc3[nH]2)cc1-c1noc(=O)[nH]1] | 3-(5-((4,6-difluoro-5-(4-(1- hydroxycyclobutyl)phenyl)-1H- indol-2-yl)oxy)-2- methylphenyl)-1,2,4-oxadiazol- 5(4H)-one |
| II-10 | 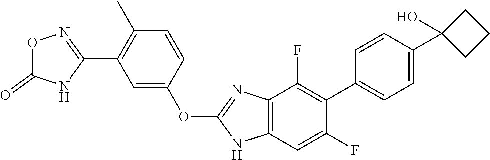 [SMILES: Cc1ccc(Oc2nc3c(F)c(-c4ccc(C5(O)CCC5)cc4)c(F)cc3[nH]2)cc1-c1noc(=O)[nH]1] | 3-(5-((4,6-difluoro-5-(4-(1- hydroxycyclobutyl)phenyl)-1H- benzo[d]imidazol-2-yl)oxy)-2- methylphenyl)-1,2,4-oxadiazol- 5(4H)-one |
| II-11 | 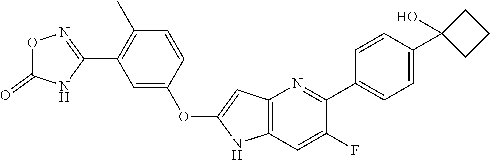 [SMILES: Cc1ccc(Oc2cc3nc(-c4ccc(C5(O)CCC5)cc4)c(F)cc3[nH]2)cc1-c1noc(=O)[nH]1] | 3-(5-((6-fluoro-5-(4-(1- hydroxycyclobutyl)phenyl)-1H- pyrrolo[3,2-b]41yridine-2- yl)oxy)-2-methylphenyl)-1,2,4- oxadiazol-5(4H)-one |
| II-12 | 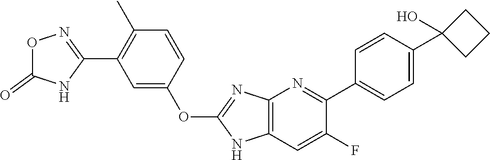 [SMILES: Cc1ccc(Oc2nc3nc(-c4ccc(C5(O)CCC5)cc4)c(F)cc3[nH]2)cc1-c1noc(=O)[nH]1] | 3-(5-((6-fluoro-5-(4-(1- hydroxycyclobutyl)phenyl)-1H- imidazo[4,5-b]pyridin-2-yl)oxy)- 2-methylphenyl)-1,2,4- oxadiazol-5(4H)-one |
| II-13 | 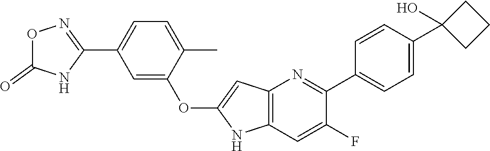 [SMILES: Cc1ccc(-c2noc(=O)[nH]2)cc1Oc1cc2nc(-c3ccc(C4(O)CCC4)cc3)c(F)cc2[nH]1] | 3-(3-((6-fluoro-5-(4-(1- hydroxycyclobutyl)phenyl)-1H- pyrrolo[3,2-b]pyridin-2-yl)oxy)- 4-methylphenyl)-1,2,4- oxadiazol-5(4H)-one |
| II-14 | 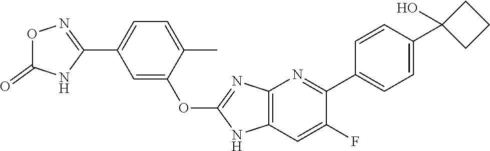 [SMILES: Cc1ccc(-c2noc(=O)[nH]2)cc1Oc1nc2nc(-c3ccc(C4(O)CCC4)cc3)c(F)cc2[nH]1] | 3-(3-((6-fluoro-5-(4-(1- hydroxycyclobutyl)phenyl)-1H- imidazo[4,5-b]pyridin-2-yl)oxy)- 4-methylphenyl)-1,2,4- oxadiazol-5(4H)-one |
| II-15 | 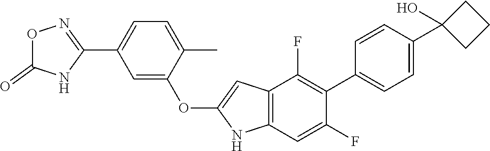 [SMILES: Cc1ccc(-c2noc(=O)[nH]2)cc1Oc1cc2c(F)c(-c3ccc(C4(O)CCC4)cc3)c(F)cc2[nH]1] | 3-(3-((4,6-difluoro-5-(4-(1- hydroxycyclobutyl)phenyl)-1H- indol-2-yl)oxy)-4- methylphenyl)-1,2,4-oxadiazol- 5(4H)-one |
| II-16 | 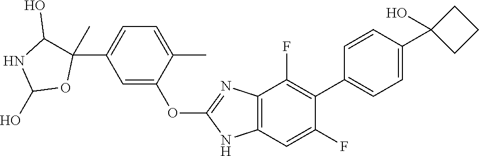 [SMILES: Cc1ccc(C2(C)OC(O)NC2O)cc1Oc1nc2c(F)c(-c3ccc(C4(O)CCC4)cc3)c(F)cc2[nH]1] | 5-(3-((4,6-difluoro-5-(4-(1- hydroxycyclobutyl)phenyl)-1H- benzo[d]imidazol-2-yl)oxy)-4- methylphenyl)-5- methyloxazolidine-2,4-diol |
| II-17 | 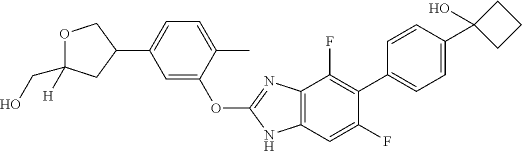 [SMILES: Cc1ccc(C2COC(CO)C2)cc1Oc1nc2c(F)c(-c3ccc(C4(O)CCC4)cc3)c(F)cc2[nH]1] | 1-(4-(4,6-difluoro-2-(5-(5- (hydroxymethyl)tetrahydrofuran- 3-yl)-2-methylphenoxy)-1H- benzo[d]imidazol-5- yl)phenyl)cyclobutan-1-ol |
| II-18 | 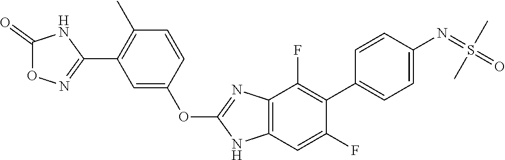 [SMILES: Cc1ccc(Oc2nc3c(F)c(-c4ccc(N=S(C)(C)=O)cc4)c(F)cc3[nH]2)cc1-c1noc(=O)[nH]1] | 3-(5-((5-(4-((dimethyl(oxo)-λ6- sulfaneylidene)amino)phenyl)- 4,6-difluoro-1H- benzo[d]imidazol-2-yl)oxy)-2- methylphenyl)-1,2,4-oxadiazol- 5(4H)-one |
| II-19 | 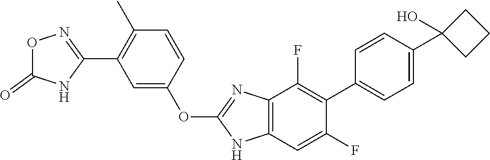 [SMILES: Cc1ccc(Oc2nc3c(F)c(-c4ccc(C5(O)CCC5)cc4)c(F)cc3[nH]2)cc1-c1noc(=O)[nH]1] | 3-(5-((4,6-difluoro-5-(4-(1- hydroxycyclobutyl)phenyl)-1H- benzo[d]imidazol-2-yl)oxy)-2- methylphenyl)-1,2,4-oxadiazol- 5(4H)-one |
| II-20 | 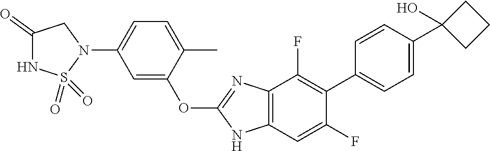 [SMILES: Cc1ccc(N2CC(=O)NS2(=O)=O)cc1Oc1nc2c(F)c(-c3ccc(C4(O)CCC4)cc3)c(F)cc2[nH]1] | 5-(3-((4,6-difluoro-5-(4-(1- hydroxycyclobutyl)phenyl)-1H- benzo[d]imidazol-2-yl)oxy)-4- methylphenyl)-1,2,5- thiadiazolidin-3-one 1,1-dioxide |
| II-21 | 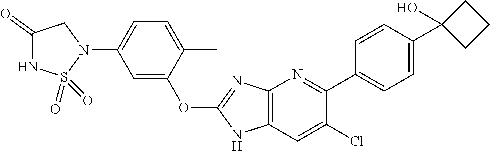 [SMILES: Cc1ccc(N2CC(=O)NS2(=O)=O)cc1Oc1nc2nc(-c3ccc(C4(O)CCC4)cc3)c(Cl)cc2[nH]1] | 5-(3-((6-chloro-5-(4-(1- hydroxycyclobutyl)phenyl)-1H- imidazo[4,5-b]pyridin-2-yl)oxy)- 4-methylphenyl)-1,2,5- thiadiazolidin-3-one 1,1-dioxide |
| II-22 | 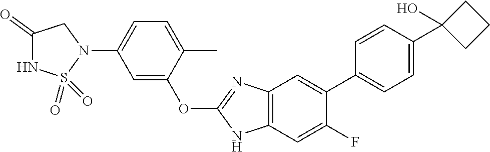 [SMILES: Cc1ccc(N2CC(=O)NS2(=O)=O)cc1Oc1nc2cc(-c3ccc(C4(O)CCC4)cc3)c(F)cc2[nH]1] | 35-(3-((6-fluoro-5-(4-(1- hydroxycyclobutyl)phenyl)-1H- benzo[d]imidazol-2-yl)oxy)-4- methylphenyl)-1,2,5- thiadiazolidin-3-one 1,1-dioxide |
| II-23 | 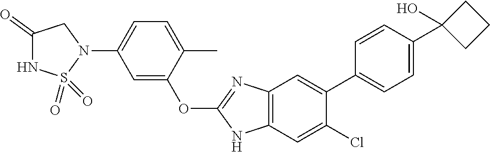 [SMILES: Cc1ccc(N2CC(=O)NS2(=O)=O)cc1Oc1nc2cc(-c3ccc(C4(O)CCC4)cc3)c(Cl)cc2[nH]1] | 5-(3-((6-chloro-5-(4-(1- hydroxycyclobutyl)phenyl)-1H- benzo[d]imidazol-2-yl)oxy)-4- methylphenyl)-1,2,5- thiadiazolidin-3-one 1,1-dioxide |
| II-24 | 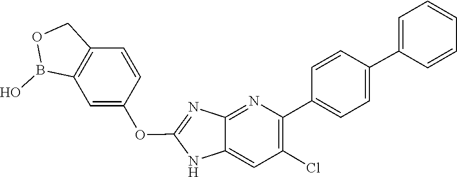 [SMILES: OB1OCc2ccc(Oc3nc4nc(-c5ccc(-c6ccccc6)cc5)c(Cl)cc4[nH]3)cc21] | 6-((5-([1,1′-biphenyl]-4-yl)-6- chloro-1H-imidazo[4,5- b]pyridin-2- yl)oxy)benzo[c][1,2]oxaborol- 1(3H)-ol |
| II-25 | 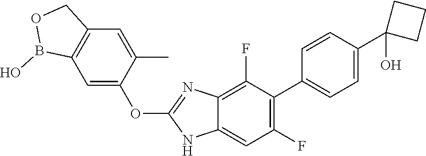 [SMILES: Cc1cc2c(cc1Oc1nc3c(F)c(-c4ccc(C5(O)CCC5)cc4)c(F)cc3[nH]1)B(O)OC2] | 6-((4,6-difluoro-5-(4-(1- hydroxycyclobutyl)phenyl)-1H- benzo[d]imidazol-2-yl)oxy)-5- methylbenzo[c][1,2]oxaborol- 1(3H)-ol |
| II-26 | 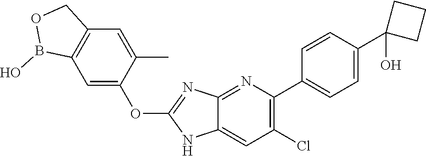 [SMILES: Cc1cc2c(cc1Oc1nc3nc(-c4ccc(C5(O)CCC5)cc4)c(Cl)cc3[nH]1)B(O)OC2] | 6-((6-chloro-5-(4-(1- hydroxycyclobutyl)phenyl)-1H- imidazo[4,5-b]pyridin-2-yl)oxy)- 5-methylbenzo[c][1,2]oxaborol- 1(3H)-ol |
| II-27 | 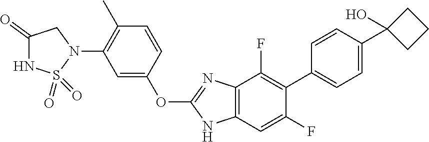 [SMILES: Cc1ccc(Oc2nc3c(F)c(-c4ccc(C5(O)CCC5)cc4)c(F)cc3[nH]2)cc1N1CC(=O)NS1(=O)=O] | 5-(5-((4,6-difluoro-5-(4-(1- hydroxycyclobutyl)phenyl)-1H- benzo[d]imidazol-2-yl)oxy)-2- methylphenyl)-1,2,5- thiadiazolidin-3-one 1,1-dioxide |
| II-28 | 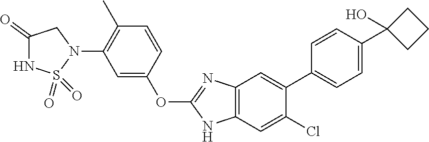 [SMILES: Cc1ccc(Oc2nc3cc(-c4ccc(C5(O)CCC5)cc4)c(Cl)cc3[nH]2)cc1N1CC(=O)NS1(=O)=O] | 5-(5-((6-chloro-5-(4-(1- hydroxycyclobutyl)phenyl)-1H- benzo[d]imidazol-2-yl)oxy)-2- methylphenyl)-1,2,5- thiadiazolidin-3-one 1,1-dioxide |

One embodiment provides a compound having the following Structure (III):

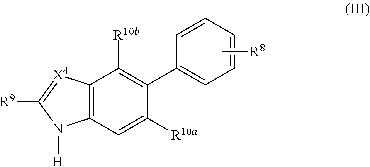

or a pharmaceutically acceptable salt, stereoisomer, or tautomer thereof, wherein:
X⁴ is N or CH;
R⁸ is optionally substituted C₃-C₆ cycloalkyl or —N═S(R^{8a})₂═O wherein R^{8a} is, at each occurrence, independently C₁-C₄ alkyl;
R⁹ is optionally substituted 5-membered heterocyclyl; and
R^{10a} and R^{10b} are each independently hydrogen or halo.

In some embodiments, R⁸ is C₃-C₆ cycloalkyl optionally substituted with —OH. In certain embodiments, R⁸ has the following structure:

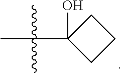 [SMILES: *C1(O)CCC1]

In certain embodiments, R⁸ is —N═S(R^{8a})₂═O and both occurrences of R^{8a} are —CH₃. In some embodiments, R⁹ has the following structure:

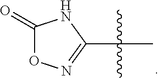 [SMILES: *c1noc(=O)[nH]1]

In certain embodiments, R^{10a} and R^{10b} are both fluoro.

In various embodiments, the compound has one of the structures set forth in Table 1C below, or a pharmaceutically acceptable salt, stereoisomer, tautomer, solvate, polymorph, isotopologue, hydrate, or prodrug thereof. Compounds in Table 1C were prepared as described in the Examples or methods known in the art and analyzed by mass spectrometry and/or ¹H NMR.

TABLE 1C
Representative compounds of Structure (III)

| No. | Structure | Name |
|---|---|---|
| III-1 | 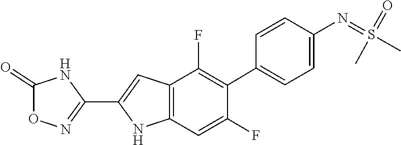 [SMILES: CS(C)(=O)=Nc1ccc(-c2c(F)cc3[nH]c(-c4noc(=O)[nH]4)cc3c2F)cc1] | 3-(5-(4-((dimethyl(oxo)-l6- sulfaneylidene)amino)phenyl)- 4,6-difluoro-1H-indol-2-yl)- 1,2,4-oxadiazol-5(4H)-one |
| III-2 | 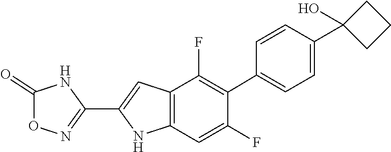 [SMILES: O=c1[nH]c(-c2cc3c(F)c(-c4ccc(C5(O)CCC5)cc4)c(F)cc3[nH]2)no1] | 3-(4,6-difluoro-5-(4-(1- hydroxycyclobutyl)phenyl)-1H- indol-2-yl)-1,2,4-oxadiazol- 5(4H)-one |
| III-3 | 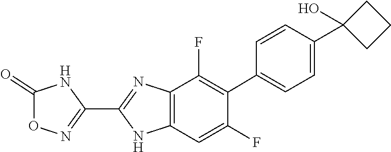 [SMILES: O=c1[nH]c(-c2nc3c(F)c(-c4ccc(C5(O)CCC5)cc4)c(F)cc3[nH]2)no1] | 3-(4,6-difluoro-5-(4-(1- hydroxycyclobutyl)phenyl)-1H- benzo[d]imidazol-2-yl)-1,2,4- oxadiazol-5(4H)-one |

One embodiment provides a compound having the following Structure (IV):

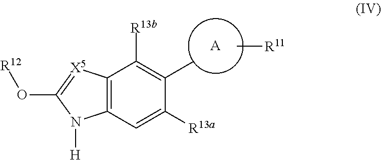

or a pharmaceutically acceptable salt, stereoisomer, or tautomer thereof, wherein:
ring A is phenyl or dihyroindenyl;
X⁵ is N or CH;
R^{11a} is optionally substituted C₃-C₆ cycloalkyl or —N═S(R^{11a})₂═O wherein R^{11a} is, at each occurrence, independently C₁-C₄ alkyl;
R¹² is optionally substituted 5-membered heterocyclyl; and
R^{13a} and R^{13b} are each independently hydrogen or halo.

In some embodiments, X⁵ is N. In certain embodiments, R¹¹ is C₃-C₆ cycloalkyl optionally substituted with —OH. In certain embodiments, R¹¹ has the following structure:

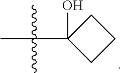 [SMILES: *C1(O)CCC1]

In some embodiments, R¹¹ is —N═S(R^{11a})₂═O and both occurrences of R^{11a} are —CH₃.

In certain embodiments,

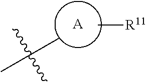

has one of the following structures:

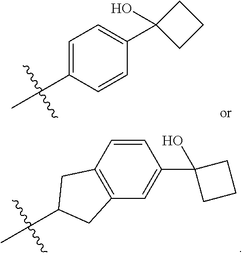 [SMILES: *c1ccc(C2(O)CCC2)cc1 ; *C1Cc2ccc(C3(O)CCC3)cc2C1]

In some embodiments, R¹² has one of the following structures:

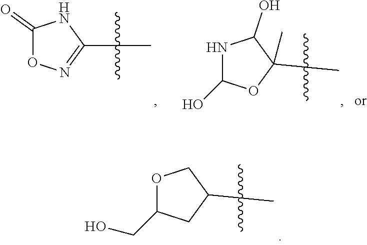

In certain embodiments, R^{13a} and R^{13b} are both fluoro.

In various embodiments, the compound has one of the structures set forth in Table 1D below, or a pharmaceutically acceptable salt, stereoisomer, tautomer, solvate, polymorph, isotopologue, hydrate, or prodrug thereof. Compounds in Table 1D were prepared as described in the Examples or methods known in the art and analyzed by mass spectrometry and/or ¹H NMR.

TABLE ID
Representative compounds of Structure (IV)

| No. | Structure | Name |
|---|---|---|
| IV-1 | 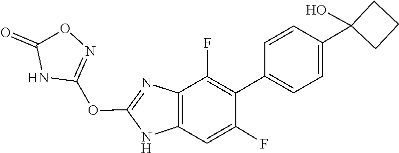 [SMILES: O=c1[nH]c(Oc2nc3c(F)c(-c4ccc(C5(O)CCC5)cc4)c(F)cc3[nH]2)no1] | 3-((4,6-difluoro-5-(4-(1- hydroxycyclobutyl)phenyl)-1H- benzo[d]imidazol-2-yl)oxy)- 1,2,4-oxadiazol-5(4H)-one |
| IV-2 | 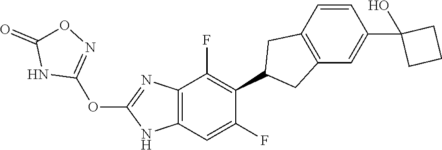 | (S)-3-((4,6-difluoro-5-(5-(1- hydroxycyclobutyl)-2,3-dihydro- 1H-inden-2-yl)-1H- benzo[d]imidazol-2-yl)oxy)- 1,2,4-oxadiazol-5(4H)-one |
| IV-3 | 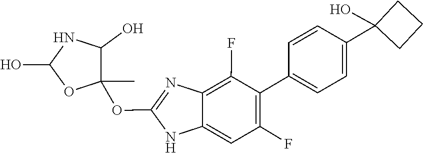 [SMILES: CC1(Oc2nc3c(F)c(-c4ccc(C5(O)CCC5)cc4)c(F)cc3[nH]2)OC(O)NC1O] | 5-((4,6-difluoro-5-(4-(1- hydroxycyclobutyl)phenyl)-1H- benzo[d]imidazol-2-yl)oxy)-5- methyloxazolidine-2,4-diol |
| IV-4 | 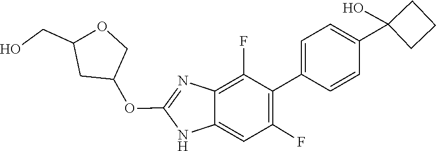 [SMILES: OCC1CC(Oc2nc3c(F)c(-c4ccc(C5(O)CCC5)cc4)c(F)cc3[nH]2)CO1] | 1-(4-(4,6-difluoro-2-((5- (hydroxymethyl)tetrahydrofuran- 3-yl)oxy)-1H-benzo[d]imidazol- 5-yl)phenyl)cyclobutan-1-ol |
| IV-5 | 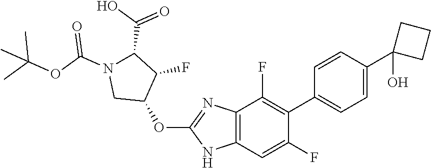 [SMILES: CC(C)(C)OC(=O)N1C[C@@H](Oc2nc3c(F)c(-c4ccc(C5(O)CCC5)cc4)c(F)cc3[nH]2)[C@@H](F)[C@H]1C(=O)O] | (2R,3S,4R)-1-(tert- butoxycarbonyl)-4-((4,6- difluoro-5-(4-(1- hydroxycyclobutyl)phenyl)-1H- benzo[d]imidazol-2-yl)oxy)-3- fluoropyrrolidine-2-carboxylic acid |

It is understood that in the present description, combinations of substituents and/or variables of the depicted formulae are permissible only if such contributions result in stable compounds.

In an additional embodiment, various compounds of the disclosure which exist in free base or acid form can be converted to their pharmaceutically acceptable salts by treatment with the appropriate inorganic or organic base or acid by methods known to one skilled in the art. Salts of the compounds of the disclosure can be converted to their free base or acid form by standard techniques.

Methods for producing the compounds described herein is provided below. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)) or prepared as described herein.

It will also be appreciated by those skilled in the art that in the processes for preparing the compounds described herein the functional groups of intermediate compounds may need to be protected by suitable protecting groups. Such functional groups include, but are not limited to, hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups are optionally added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this disclosure may not possess pharmacological activity as such, they may be administered to a mammal and thereafter metabolized in the body to form compounds of the disclosure which are pharmacologically active. Such derivatives may therefore be described as “prodrugs.” Prodrugs of compounds of this disclosure are included within the scope of embodiments of the disclosure.

### Pharmaceutical Compositions

Other embodiments are directed to pharmaceutical compositions. The pharmaceutical composition comprises anyone (or more) of the foregoing compounds and a pharmaceutically acceptable carrier. In some embodiments, the pharmaceutical composition is formulated for oral administration. In other embodiments, the pharmaceutical composition is formulated for injection. In still more embodiments, the pharmaceutical compositions comprise a compound as disclosed herein and an additional therapeutic agent (e.g., anticancer agent). Non-limiting examples of such therapeutic agents are described herein below.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long-acting formulations are administered by implantation (for example subcutaneously or intramuscularly) or by intramuscular injection. Furthermore, in other embodiments, the compound is delivered in a targeted drug delivery system, for example, in a liposome coated with and organ-specific antibody. In such embodiments, the liposomes are targeted to and taken up selectively by the organ. In yet other embodiments, the compound as described herein is provided in the form of a rapid release formulation, in the form of an extended-release formulation, or in the form of an intermediate release formulation. In yet other embodiments, the compound described herein is administered topically.

In treatment methods according to embodiments of the disclosure, an effective amount of at least one compound of Structure (I), (II), (III), or (IV) is administered to a subject suffering from or diagnosed as having such a disease, disorder, or medical condition. Effective amounts or doses may be ascertained by methods such as modeling, dose escalation studies or clinical trials, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician.

The compounds according to the disclosure are effective over a wide dosage range. For example, in the treatment of adult humans, dosages from 10 to 5000 mg, from 100 to 5000 mg, from 1000 mg to 4000 mg per day, and from 1000 to 3000 mg per day are examples of dosages that are used in some embodiments. The exact dosage will depend upon the route of administration, the form in which the compound is administered, the subject to be treated, the body weight of the subject to be treated, and the preference and experience of the attending physician.

In some embodiments, compounds of the disclosure are administered in a single dose. Typically, such administration will be by injection, e.g., intravenous injection, to introduce the agent quickly. However, other routes are used as appropriate. A single dose of a compound of the disclosure may also be used for treatment of an acute condition.

In some embodiments, compounds of the disclosure are administered in multiple doses. In some embodiments, dosing is about once, twice, three times, four times, five times, six times, or more than six times per day. In other embodiments, dosing is about once a month, once every two weeks, once a week, or once every other day. In another embodiment compounds of the disclosure and another agent (e.g., anti-cancer agent) are administered together about once per day to about 6 times per day. In another embodiment the administration of compounds of the disclosure and an agent continues for less than about 7 days. In yet another embodiment the administration continues for more than about 6, 10, 14, 28 days, two months, six months, or one year. In some cases, continuous dosing is achieved and maintained as long as necessary.

Administration of compounds of the disclosure may continue as long as necessary. In some embodiments, compounds of the disclosure are administered for more than 1, 2, 3, 4, 5, 6, 7, 14, or 28 days. In some embodiments, compounds of the disclosure are administered for less than 28, 14, 7, 6, 5, 4, 3, 2, or 1 day. In some embodiments, compounds of the disclosure are administered chronically on an ongoing basis, e.g., for the treatment of chronic effects.

In some embodiments, the compounds of the disclosure are administered in individual dosage forms. It is known in the art that due to intersubject variability in compound pharmacokinetics, individualization of dosing regimen is necessary for optimal therapy.

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the disclosed compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising one or more compounds of Structure (I), (II), (III), or (IV), and a pharmaceutically acceptable carrier.

Provided herein are pharmaceutical compositions comprising one or more compounds selected from compounds of Structure (I), (II), (III), or (IV) and pharmaceutically acceptable diluent(s), excipient(s), and carrier(s). In certain embodiments, the compounds described are administered as pharmaceutical compositions in which one or more compounds selected from compounds of Structure (I), (II), (III), or (IV) are mixed with other active ingredients, as in combination therapy. Encompassed herein are all combinations of actives set forth in the combination therapies section below and throughout this disclosure. In specific embodiments, the pharmaceutical compositions include one or more compounds of Structure (I), (II), (III), or (IV).

In a certain embodiment, pharmaceutical compositions of the compounds of Structure (I), (II), (III), or (IV) are modulators of AMPK. In some embodiments, pharmaceutical compositions of compounds of Structure (I), (II), (III), or (IV) are activators for AMPK.

A pharmaceutical composition, as used herein, refers to a mixture of one or more compounds selected from compounds of Structure (I), (II), (III), or (IV) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, therapeutically effective amounts of one or more compounds selected from compounds of Structure (I), (II), (III), or (IV) provided herein are administered in a pharmaceutical composition to a mammal having a disease, disorder, or medical condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, one or more compounds selected from compounds of Structure (I), (II), (III), or (IV) are formulated in aqueous solutions. In specific embodiments, the aqueous solution is selected from, by way of example only, a physiologically compatible buffer, such as Hank's solution, Ringer's solution, or physiological saline buffer. In other embodiments, one or more compounds selected from compounds of Structure (I), (II), (III), or (IV) are formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or non-aqueous solutions. In specific embodiments, such solutions include physiologically compatible buffers and/or excipients.

In another embodiment, compounds described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions, and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In still other embodiments, the compounds described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions are formulated in a form suitable for parenteral injection as sterile suspensions, solutions, or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of one or more compounds selected from compounds of Structure (I), (II), (III), or (IV) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

Pharmaceutical compositions include at least one pharmaceutically acceptable carrier, diluent, or excipient, and one or more compounds selected from compounds of Structure (I), (II), (III), or (IV), described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions, and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical compositions comprising one or more compounds selected from compounds of Structure (I), (II), (III), or (IV) illustratively takes the form of a liquid where the agents are present in solution, in suspension or both. Typically, when the composition is administered as a suspension, a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, aqueous suspensions contain one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally, include solubilizing agents to aid in the solubility of one or more compounds selected from compounds of Structure (I), (II), (III), or (IV). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric, and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate, or bisulfite anions; suitable salts include sodium chloride, potassium chloride, sodium thiosulfate, sodium bisulfite, and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide, and cetylpyridinium chloride.

Compositions may include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Compositions may include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

In some embodiments, the concentration of one or more compounds selected from compounds of Structure (I), (II), (III), or (IV) provided in the pharmaceutical compositions of the present disclosure is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more compounds selected from compounds of Structure (I), (II), (III), or (IV) provided in the pharmaceutical compositions of the present disclosure is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the amount the one or more compounds selected from compounds of Structure (I), (II), (III), or (IV) provided in the pharmaceutical compositions of the present disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the one or more compounds selected from compounds of Structure (I), (II), (III), or (IV) provided in the pharmaceutical compositions of the present disclosure is in the range of 0.0001-10 g, 0.0005-9 g, 0.001-8 g, 0.005-7 g, 0.01-6 g, 0.05-5 g, 0.1-4 g, 0.5-4 g, or 1-3 g.

Packaging materials for use in packaging pharmaceutical compositions described herein include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more compounds described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded, or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical compositions are presented in a pack or dispenser device which contains one or more unit dosage forms containing a compound provided herein.

For example, the pack may (1) contain metal or plastic foil (e.g., a blister pack), (2) be accompanied by instructions for administration, (3) be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound of Structure (I), (II), (III), or (IV) formulated in a compatible pharmaceutical carrier are prepared, placed in an appropriate container, and labeled for treatment of an indicated condition.

### Methods

Embodiments of the present disclosure provide compounds that are useful as AMPK modulators in a host species. Therefore, the compounds of Structure (I), (II), (III), or (IV) are also useful in the treatment of conditions mediated by AMPK.

The host or patient can belong to any mammalian species, for example a primate species, particularly humans; rodents, including mice, rats, hamsters, rabbits, horses, cows, dogs, cats, etc. Animal models are of interest for experimental investigations, providing a model for treatment of human disease.

In one embodiment, the present disclosure is useful as a modulator of AMPK. Therefore, the compounds of Structure (I), (II), (III), or (IV) are also useful in the treatment of conditions resulting from under expression of AMPK activity.

Embodiments also relate to the use of compounds according to Structure (I), (II), (III), or (IV) and/or physiologically acceptable salts thereof for the prophylactic or therapeutic treatment and/or monitoring of diseases that are caused, mediated, and/or modulated by AMPK. Furthermore, embodiments relate to the use of compounds according to Structure (I), (II), (III), or (IV) and/or physiologically acceptable salts thereof to produce a medicament for the prophylactic or therapeutic treatment and/or monitoring of diseases that are caused, mediated, and/or modulated by AMPK. In certain embodiments, the disclosure provides the use of a compound according to Structure (I), (II), (III), or (IV) or physiologically acceptable salts thereof, to produce a medicament for the prophylactic or therapeutic treatment of an AMPK-mediated disorder.

In another embodiment, the present disclosure relates to a method of treating diseases or conditions mediated by AMPK by administering to a patient in need thereof a therapeutically effective amount of the compound of Structure (I), (II), (III), or (IV).

One embodiment provides a method of treating an AMPK mediated disease, the method comprising administering the compound of Structure (I), (II), (III), or (IV), or a pharmaceutically acceptable salt, stereoisomer, or tautomer thereof or a pharmaceutical composition comprising a compound of Structure (I), (II), (III), or (IV).

In some embodiments, the AMPK mediated disease is a cardiometabolic disease, a neuromuscular disorder, a cancer, a neurodegenerative disease, or combinations thereof. In certain embodiments, the AMPK mediated disease is obesity, diabetes, chronic inflammation, cardiac energy homeostasis, ischemia-reperfusion injury, endothelial dysfunction, dyslipidemia, cardiac hypertrophy and remodeling, Duchenne muscular dystrophy (DMD), myotonic dystrophy type 1 (DM1), spinal muscular atrophy (SMA), Non-alcoholic Fatty Liver Disease (NAFLD), Alzheimer's disease, or combinations thereof.

Also included herein are methods of treatment in which at least one compound of Structure (I), (II), (III), or (IV) is administered in combination with an anti-inflammatory or a therapeutic agent. Anti-inflammatory agents include but are not limited to NSAIDs, non-specific and COX-2 specific cyclooxygenase enzyme inhibitors, gold compounds, corticosteroids, methotrexate, tumor necrosis factor (TNF) antagonists, immunosuppressants and methotrexate. Examples of NSAIDs include, but are not limited to, ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, and hydroxychloroquine.

Still other embodiments of the disclosure pertain to combinations in which at least one active agent is an immunosuppressant compound such as an immunosuppressant compound chosen from methotrexate, leflunomide, cyclosporine, tacrolimus, azathioprine, and mycophenolate mofetil.

The disclosed compounds of Structure (I), (II), (III), or (IV) can be administered in combination with other known therapeutic agents, including anticancer agents. As used here, the term “anticancer agent” relates to any agent which is administered to a patient with cancer for the purposes of treating the cancer.

In some embodiments the anti-cancer agents belong to the following categories
Alkylating agents: such as altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone; apaziquone, fotemustine, glufosfamide, palifosfamide, pipobroman, trofosfamide, uramustine, TH-3024, VAL-0834;
Platinum Compounds: such as carboplatin, cisplatin, eptaplatin, miriplatine hydrate, oxaliplatin, lobaplatin, nedaplatin, picoplatin, satraplatin; lobaplatin, nedaplatin, picoplatin, satraplatin;
DNA altering agents: such as amrubicin, bisantrene, decitabine, mitoxantrone, procarbazine, trabectedin, clofarabine; amsacrine, brostallicin, pixantrone, or laromustine; Topoisomerase Inhibitors: such as etoposide, irinotecan, razoxane, sobuzoxane, teniposide, topotecan; amonafide, belotecan, elliptinium acetate, or voreloxin;
Microtubule modifiers: such as cabazitaxel, docetaxel, eribulin, ixabepilone, paclitaxel, vinblastine, vincristine, vinorelbine, vindesine, vinflunine; fosbretabulin, or tesetaxel;
Antimetabolites: such as asparaginase3, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur; doxifluridine, elacytarabine, raltitrexed, sapacitabine, tegafur, or trimetrexate;
Anticancer antibiotics: such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin; aclarubicin, peplomycin, or pirarubicin; Hormones/Antagonists: such as abarelix, abiraterone, bicalutamide, buserelin, calusterone, chlorotrianisene, degarelix, dexamethasone, estradiol, fluocortolone fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol; acolbifene, danazol, deslorelin, epitiostanol, orteronel, or enzalutamide;
Aromatase inhibitors: such as aminoglutethimide, anastrozole, exemestane, fadrozole, letrozole, testolactone, or formestane;
Small molecule kinase inhibitors: such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib; afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifamib, tivantinib, tivozanib, trametinib, pimasertib, brivanib alaninate, or cediranib.

In some embodiments, medicaments which are administered in conjunction with the compounds described herein include any suitable drugs usefully delivered by inhalation for example, analgesics, e.g. codeine, dihydromorphine, ergotamine, fentanyl or morphine; anginal preparations, e.g. diltiazem; antiallergics, e.g. cromoglycate, ketotifen or nedocromil; anti-infectives, e.g. cephalosporins, penicillins, streptomycin, sulphonamides, tetracyclines or pentamidine; antihistamines, e.g. methapyrilene; anti-inflammatories, e.g. beclomethasone, flunisolide, budesonide, tipredane, triamcinolone acetonide or fluticasone; antitussives, e.g. noscapine; bronchodilators, e.g. ephedrine, adrenaline, fenoterol, formoterol, isoprenaline, metaproterenol, phenylephrine, phenylpropanolamine, pirbuterol, reproterol, rimiterol, salbutamol, salmeterol, terbutalin, isoetharine, tulobuterol, orciprenaline or (−)-4-amino-3,5-dichloro-α-[[[6-[2-(2-pyridinyl)ethoxy]hexyl]-amino]methyl]benzenemethanol; diuretics, e.g., amiloride; anticholinergics, e.g., ipratropium, atropine or oxitropium; hormones, e.g., cortisone, hydrocortisone or prednisolone; xanthines, e.g., aminophylline, choline theophyllinate, lysine theophyllinate or theophylline; and therapeutic proteins and peptides, e.g., insulin. It will be clear to a person skilled in the art that, where appropriate, the medicaments are used in the form of salts (e.g., as alkali metal or amine salts or as acid addition salts) or as esters (e.g., lower alkyl esters) or as solvates (e.g., hydrates) to optimize the activity and/or stability of the medicament.

The agents disclosed herein, or other suitable agents are administered depending on the condition being treated. Hence, in some embodiments the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the disclosure and any of the agents described above can be simultaneously administered, wherein both the agents are present in separate formulations. In another alternative, a compound of the present disclosure can be administered just followed by and any of the agents described above, or vice versa. In some embodiments of the separate administration protocol, a compound of the disclosure and any of the agents described above are administered a few minutes apart, or a few hours apart, or a few days apart.

In some embodiments, the compounds of Structure (I), (II), (III), or (IV) are administered as a monotherapy.

In some embodiments, the methods of the disclosure can be performed either in vitro, in vivo, or as a combination thereof. The susceptibility of a particular cell to treatment with the compounds of Structure (I), (II), (III), or (IV) can be particularly determined by in vitro tests, whether during research or clinical application. Typically, a culture of the cell is combined with a compound at various concentrations for a period which is sufficient to allow the active agents to modulate AMPK activity, usually between about one hour and one week. In vitro treatment can be carried out using cultivated cells from a biopsy sample or cell line.

In some embodiments, the IC₅₀ of the compounds of Structure (I), (II), (III), or (IV) to modulate AMPK was determined by the concentration of the compound required to modulate 50% of the activity of AMPK.

It is understood that one skilled in the art may be able to make these compounds by similar methods or by combining other methods known to one skilled in the art. It is also understood that one skilled in the art would be able to make, in a similar manner as described below, other compounds of Structure (I), (II), (III), or (IV) not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)) or prepared as described in this disclosure.

The following examples are provided for purpose of illustration and not limitation.

### Abbreviations:

° C. (degree Celsius); ¹H NMR (proton Nuclear Magnetic Resonance); DCM (dichloromethane); DMSO (dimethylsulfoxide); eq (equivalent); EtOAc (ethyl acetate); g (gram); h (hour); MeOH (methanol); mg (milligram); min (minute); mL (milliliter); mmol (millimole); TFA (trifluoroacetic acid); THF (tetrahydrofuran); TLC (Thin Layer Chromatography); LDA (lithium diisopropylamide); AcOH (acetic acid); mCPBA (3-chloroperbenzoic acid).

### SYNTHETIC EXAMPLE 1

### Synthesis of 3-(6-chloro-5-(4-(1-hydroxycyclobutyl)phenyl)-1H-indol-3-yl)-1,2,4-oxadiazol-5(4H)-one

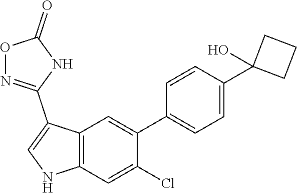 [SMILES: O=c1[nH]c(-c2c[nH]c3cc(Cl)c(-c4ccc(C5(O)CCC5)cc4)cc23)no1]

### Synthesis of 1-(4-(4,4,5,5-tetramethyl-1,3,2-dioxaborolan-2-yl)phenyl)cyclobutan-1-ol

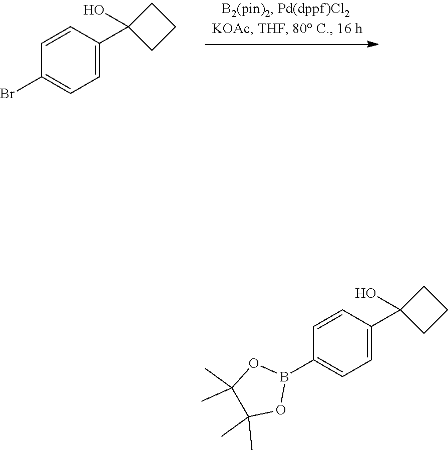 [SMILES: OC1(c2ccc(Br)cc2)CCC1 ; CC1(C)OB(c2ccc(C3(O)CCC3)cc2)OC1(C)C]

In a sealed tube to a degassed solution of 1-(4-bromophenyl)cyclobutan-1-ol (500 mg, 2.20 mmol, 1.0 eq) and B₂(pin)₂ (671 mg, 2.64 mmol, 1.2 eq) in tetrahydrofuran (“THF”—10 mL) at room temperature, was added potassium acetate (“KOAc”—647 mg, 6.60 mmol, 3.0 eq), Pd(dppf)Cl₂ (80 mg, 0.11 mmol, 0.05 eq) and stirred at 80° C. for 16 h. After completion of reaction by TLC, reaction mixture was filtered through a pad of diatomaceous earth (e.g., Celite®), washed with ethyl acetate (“EtOAc”−10 mL), collected filtrate was diluted with water (20 mL) and extracted with EtOAc (2×20 mL). The combined organic layer was dried over Na₂SO₄, filtered, and concentrated under reduced pressure to afford 1-(4,4,5,5-tetramethyl-1,3,2-dioxaborolan-2-yl)phenyl)cyclobutan-1-ol as black gummy solid (500 mg, Yield: Crude).
TLC system: EtOAc/Hexane (30:70)
R_{f value}: ˜0.5

¹H NMR (400 MHz, CDCl₃) δ: 7.83 (d, J=8.0 Hz, 2H), 7.51 (d, J=8.0 Hz, 2H), 2.57-2.53 (m, 2H), 2.41-2.34 (m, 2H), 2.08-1.98 (m, 2H), 1.35 (s, 12H)

### Synthesis of 5-bromo-6-chloro-1H-indole-3-carbaldehyde

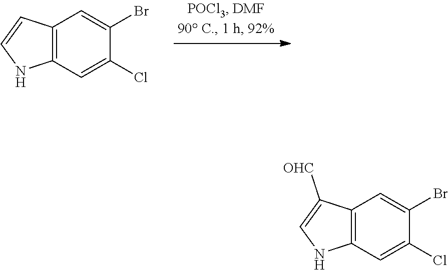 [SMILES: Clc1cc2[nH]ccc2cc1Br ; O=Cc1c[nH]c2cc(Cl)c(Br)cc12]

To a stirred solution of 5-bromo-6-chloro-1H-indole (3 g, 13.04 mmol, 1.0 eq) in DMF (3 mL) at room temperature, was added POCl₃ (2.43 mL, 26.1 mmol, 2.0 eq) and stirred at 90° C. for 1 h. After completion of reaction by TLC, reaction mixture was treated with 1N NaOH solution (10 mL) and stirred at 95° C. for 10 min. Solid precipitation observed, filtered, washed with diethyl ether (50 mL) and dried under vacuum to afford 5-bromo-6-chloro-1H-indole-3-carbaldehyde as pink solid (3.0 g, Yield: 92%).
TLC system: EtOAc/Hexane (30:70)
R_{f} value: ˜0.4;

LCMS (m/z): 259.8 (M+H)⁺

¹H NMR (400 MHz, DMSO-d₆) δ: 12.34 (s, 1H), 9.93 (s, 1H), 8.40 (s, 1H), 8.39 (s, 1H), 7.80 (s, 1H)

### Synthesis of 5-bromo-6-chloro-1H-indole-3-carbonitrile

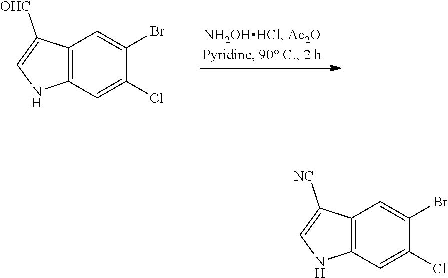 [SMILES: O=Cc1c[nH]c2cc(Cl)c(Br)cc12 ; N#Cc1c[nH]c2cc(Cl)c(Br)cc12]

To a stirred solution of 5-bromo-6-chloro-1H-indole-3-carbaldehyde (3 g, 11.5 mmol, 1.0 eq) in Pyridine (15 mL) at room temperature, was added Ac₂O (2.2 mL, 23.1 mmol, 2.0 eq), NH₂OH·HCl (888 mg, 12.7 mmol, 1.1 eq) and stirred at 90° C. for 2 h. After completion of reaction by TLC, reaction mixture was quenched with 2 N HCl solution (20 mL) and extracted with EtOAc (2×30 mL). The combined organic layer was dried over Na₂SO₄, filtered, and concentrated under reduced pressure. The obtained crude product was triturated with diethyl ether (10 mL), decanted, and dried under reduced pressure to afford 5-bromo-6-chloro-1H-indole-3-carbonitrile as an off white solid (2.2 g, Yield: Crude).
TLC system: EtOAc/Hexane (50:50)
R_{f value}: ˜0.4; LCMS (m/z): 253.0 (M−H)⁺

### Synthesis of (Z)-5-bromo-6-chloro-N′-hydroxy-1H-indole-3-carboximidamide

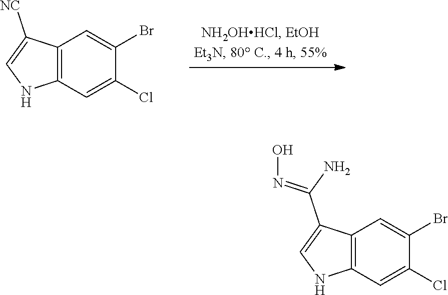 [SMILES: N#Cc1c[nH]c2cc(Cl)c(Br)cc12 ; N/C(=N\O)c1c[nH]c2cc(Cl)c(Br)cc12]

To a stirred solution of 5-bromo-6-chloro-1H-indole-3-carbonitrile (1.1 g, 4.33 mmol, 1.0 eq) in EtOH (11 mL) at room temperature, was added triethyl amine (“Et₃N”—1.0 mL, 6.49 mmol, 1.5 eq) and NH₂OH·HCl (451 mg, 6.49 mmol, 1.5 eq). The reaction mixture was stirred at 80° C. for 4 h. After completion of reaction by TLC, reaction mixture was diluted with water (30 mL) and extracted with EtOAc (2×30 mL). The combined organic layer was dried over Na₂SO₄, filtered, and concentrated under reduced pressure to afford crude product. The crude product was purified by silica gel (100-200) column chromatography [elution with 60% EtOAc in Hexane] to afford (Z)-5-bromo-6-chloro-N′-hydroxy-1H-indole-3-carboximidamide as an off white solid (720 mg, Yield: 55%).
TLC system: EtOAc/Hexane (70:30)
R_{f value}: ˜0.4

¹H NMR (400 MHz, DMSO-d₆) δ: 11.51 (s, 1H), 9.36 (s, 1H), 8.45 (s, 1H), 7.87 (d, J=2.8 Hz, 1H), 7.65 (s, 1H), 5.69 (s, 2H).

### Synthesis of 3-(5-bromo-6-chloro-1H-indol-3-yl)-1,2,4-oxadiazol-5(4H)-one

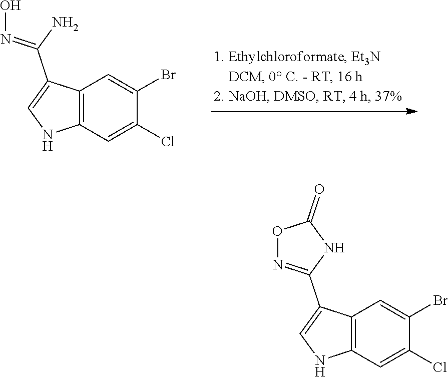 [SMILES: N/C(=N\O)c1c[nH]c2cc(Cl)c(Br)cc12 ; O=c1[nH]c(-c2c[nH]c3cc(Cl)c(Br)cc23)no1]

To a stirred solution of (Z)-5-bromo-6-chloro-N′-hydroxy-1H-indole-3-(720 mg, 2.55 mmol, 1.0 eq) in dichloromethane (“DCM”-21.6 mL, 30 Vol) at 0° C., was added Et₃N (0.5 mL, 3.31 mmol, 1.3 eq) and Ethyl chloroformate (0.3 mL, 3.06 mmol, 1.2 eq). The reaction mixture was stirred at room temperature for 16 h. After completion of reaction by TLC, e reaction mixture was quenched with NaHCO₃ solution (20 mL) and extracted with DCM (2×20 mL). The organic layer was dried over Na₂SO₄, filtered, and concentrated to afford crude. The crude was dissolved in DMSO (2.5 mL), treated with NaOH (96 mg, 2.40 mmol, 1.2 eq) and stirred for 3 h. Later, diluted with water (20 mL), quenched with 2N HCl solution (10 mL) and extracted with EtOAc (2×20 mL). The combined organic layer was dried over Na₂SO₄, filtered, and concentrated under reduced pressure to afford crude product. The crude product was purified by reverse phase column chromatography [elution with 30% acetonitrile (“ACN”) in 0.1% formic acid (“FA”) in H₂O] to afford 3-(5-bromo-6-chloro-1H-indol-3-yl)-1,2,4-oxadiazol-5(4H)-one as brick red solid (300 mg, Yield: 37%).
TLC system: EtOAc/Hexane (70:30)
R_{f value}: ˜0.3

LCMS (m/z): 314.0 (M+H)⁺

### Synthesis of 3-(6-chloro-5-(4-(1-hydroxycyclobutyl)phenyl)-1H-indol-3-yl)-1,2,4-oxadiazol-5(4H)-one

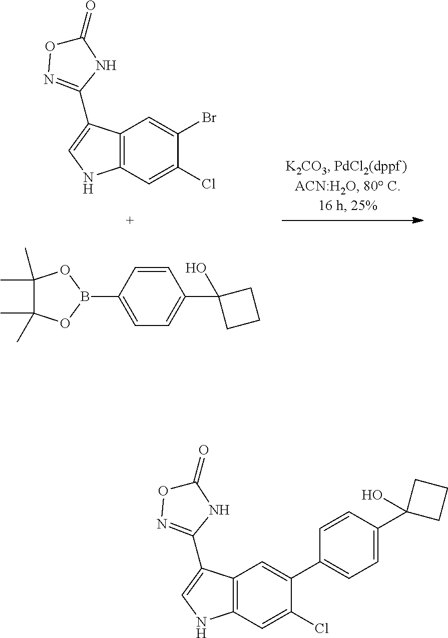 [SMILES: O=c1[nH]c(-c2c[nH]c3cc(Cl)c(Br)cc23)no1 ; CC1(C)OB(c2ccc(C3(O)CCC3)cc2)OC1(C)C ; O=c1[nH]c(-c2c[nH]c3cc(Cl)c(-c4ccc(C5(O)CCC5)cc4)cc23)no1]

In a sealed tube to a degassed solution of 3-(5-bromo-6-chloro-1H-indol-3-yl)-1,2,4-oxadiazol-5(4H)-one (200 mg, 0.64 mmol, 1.0 eq) and 1-(4-(4,4,5,5-tetramethyl-1,3,2-dioxaborolan-2-yl)phenyl)cyclobutan-1-ol (262 mg, 0.95 mmol, 1.5 eq) in ACN:H₂O (1:1, 2 mL) at room temperature, was added K₂CO₃ (176 mg, 1.28 mmol, 2.0 eq), Pd(dppf)Cl₂ (46 mg, 0.064 mmol, 0.1 eq) and stirred at 80° C. for 16 h. After completion of reaction by TLC, the reaction mixture was diluted with water (20 mL) and extracted with EtOAc (2×20 mL). The combined organic layer was dried over Na₂SO₄, filtered, and concentrated under reduced pressure to afford crude product. The crude product was purified by reverse phase column chromatography [elution with 25% ACN in 0.1% FA in H₂O] followed by Prep-HPLC and lyophilizing the fractions afforded 3-(6-chloro-5-(4-(1-hydroxycyclobutyl)phenyl)-1H-indol-3-yl)-1,2,4-oxadiazol-5(4H)-one as white solid (31 mg, Yield: 25%).
TLC system: EtOAc/Hexane (70:30)
R_{f} value: ˜0.4

LCMS (m/z): 380.0 (M−H)⁺

¹H NMR (400 MHz, DMSO-d₆) δ: 12.03 (s, 1H), 12.02 (s, 1H), 8.04 (d, J=2.8 Hz, 1H), 7.85 (s, 1H), 7.74 (s, 1H), 7.57 (d, J=8.4 Hz, 2H), 7.41 (d, J=8.0 Hz, 2H), 5.54 (s, 1H), 2.47-2.42 (m, 2H), 2.34-2.27 (m, 2H), 1.98-1.91 (m, 1H), 1.75-1.70 (m, 1H).

### SYNTHETIC EXAMPLE 2

### Synthesis of (S)-1-(6-chloro-5-(4-(1-hydroxycyclobutyl)phenyl)-1H-indol-3-yl)-4-methyl-4,5-dihydro-3H-1L6,2,5-thiadiazol-3-one 1-oxide

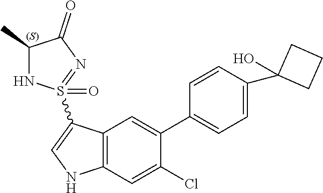

### Synthesis of 5-bromo-6-chloro-1H-indole-3-sulfonyl chloride

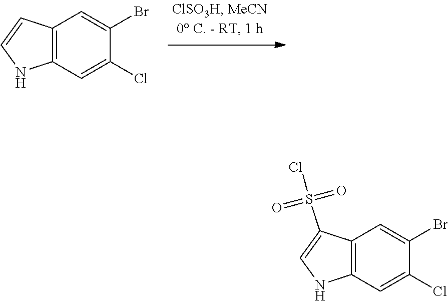 [SMILES: Clc1cc2[nH]ccc2cc1Br ; O=S(=O)(Cl)c1c[nH]c2cc(Cl)c(Br)cc12]

To a stirred solution of 5-bromo-6-chloro-1H-indole (6 g, 26.1 mmol, 1.0 eq) in MeCN (150 mL) at 0° C., was added ClSO₃H (17 mL, 31.3 mmol, 1.2 eq) and stirred at room temperature for 1 h. After observing conversion by TLC, reaction mixture was concentrated, diluted with water (70 mL) and extracted with EtOAc (2×70 mL). The combined organic layer was dried over Na₂SO₄, filtered, and concentrated under reduced pressure. The obtained crude product was triturated with n-pentane (50 mL), decanted, and dried under vacuum to afford 5-bromo-6-chloro-1H-indole-3-sulfonyl chloride as pale yellow solid (6 g, Yield: Crude).
TLC system: EtOAc/Hexane (20:80)
R_{f value}: ˜0.4

LCMS (m/z): 308.5 (M−H of sulfonic acid)⁺

¹H NMR (400 MHz, CDCl₃) δ: 9.12 (s, 1H), 8.32 (s, 1H), 7.99 (d, J=3.2 Hz, 1H), 7.66 (s, 1H).

### Synthesis of 5-bromo-6-chloro-1H-indole-3-sulfonamide

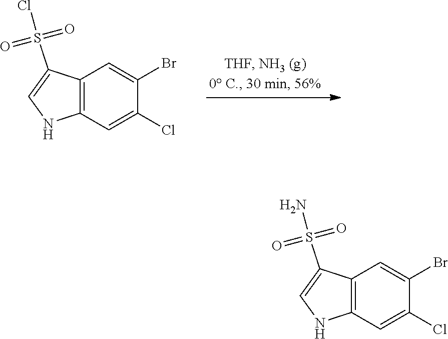 [SMILES: O=S(=O)(Cl)c1c[nH]c2cc(Cl)c(Br)cc12 ; NS(=O)(=O)c1c[nH]c2cc(Cl)c(Br)cc12]

To a stirred solution of 5-bromo-6-chloro-1H-indole-3-sulfonyl chloride (6.0 g, 18.2 mmol, 1.0 eq) in THF (120 mL) at 0° C., NH₃ gas was purged for 30 min. Later, the reaction mixture was diluted with water (70 mL) and extracted with EtOAc (2×100 mL). The combined organic layer was dried over Na₂SO₄, filtered, and concentrated under reduced pressure to afford crude product. The crude product was triturated with n-pentane (50 mL), decanted, and dried under vacuum to afford 5-bromo-6-chloro-1H-indole-3-sulfonamide as off white solid (4.5 g, Yield: 56%).
TLC system: EtOAc/Hexane (50:50)
R_{f} value: ˜0.3

LCMS (m/z): 306.7 (M−H)⁺

¹H NMR (400 MHz, CDCl₃) δ: 12.01 (s, 1H), 8.22 (s, 1H), 7.91 (s, 1H), 7.76 (s, 1H), 7.29 (s, 2H).

### Synthesis of 5-bromo-N-(tert-butyldimethylsilyl)-6-chloro-1H-indole-3-sulfonamide

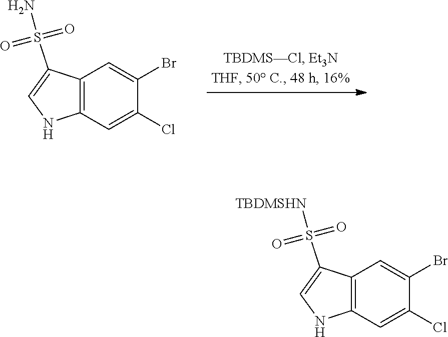

To a stirred solution of 5-bromo-6-chloro-1H-indole-3-sulfonamide (4.5 g, 14.6 mmol, 1.0 eq) in THF (50 mL) at room temperature, was added Et₃N (10.2 mL, 73.3 mmol, 5.0 eq) and TBDMS-Cl (8.8 g, 58.6 mmol, 4.0 eq). The reaction mixture was stirred at 50° C. for 48 h. After completion of reaction by TLC, the reaction mixture was diluted with ice cold water (40 mL) and extracted with EtOAc (3×40 mL). The combined organic layer was dried over Na₂SO₄, filtered, and concentrated under reduced pressure to afford crude [elution with 20-25% EtOAc in Hexane] to afford 5-bromo-N-(tert-butyldimethylsilyl)-6-chloro-1H-indole-3-sulfonamide as pale yellow solid (1.0 g, Yield: 16%).
TLC system: EtOAc/Hexane (50:50)
R_{f} value: ˜0.5

LCMS (m/z): 425.1 (M+H)⁺

¹H NMR (400 MHz, CDCl₃) δ: 8.63 (s, 1H), 8.19 (s, 1H), 7.70 (d, J=2.8 Hz, 1H), 7.57 (s, 1H), 4.40 (s, 1H), 0.89 (s, 9H), 0.22 (s, 6H).

### Synthesis of tert-butyl 5-bromo-3-(N-(tert-butyldimethylsilyl)sulfamoyl)-6-chloro-1H-indole-1-carboxylate

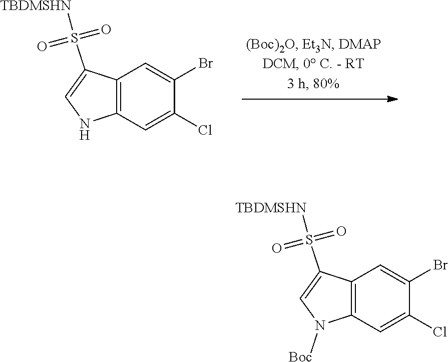

To a stirred solution of 5-bromo-N-(tert-butyldimethylsilyl)-6-chloro-1H-indole-3-sulfonamide (1.0 g, 2.37 mmol, 1.0 eq) in DCM (5 mL) at 0° C., was added Et₃N (0.8 mL, 5.92 mmol, 2.5 eq), (Boc)₂O (0.6 mL, 4.74 mmol, 2.0 eq) followed by DMAP (86 mg, 0.71 mmol, 0.3 eq). The reaction mixture was stirred at room temperature for 3 h. After completion of reaction by TLC, e reaction mixture was diluted with ice cold water (30 mL) and extracted with DCM (2×30 mL). The combined organic layer was dried over Na₂SO₄, filtered, concentrated under reduced pressure, and purified by silica gel (100-200) column chromatography [elution with 10-15% EtOAc in Hexane] to afford tert-butyl 5-bromo-3-(N-(tert-butyldimethylsilyl)sulfamoyl)-6-chloro-1H-indole-1-carboxylate as off white solid (1 g, Yield: 80%).
TLC system: EtOAc/Hexane (20:80)
R_{f} value: ˜0.6

¹H NMR (400 MHz, CDCl₃) δ: 8.41 (s, 1H), 8.18 (s, 1H), 8.07 (s, 1H), 4.54 (s, 1H), 1.72 (s, 9H), 0.96 (s, 9H), 0.30 (s, 6H).

### Synthesis of tert-butyl(S)-5-bromo-3-(N′-(tert-butyldimethylsilyl)-N-(1-methoxy-1-oxopropan-2-yl)sulfamidimidoyl)-6-chloro-1H-indole-1-carboxylat

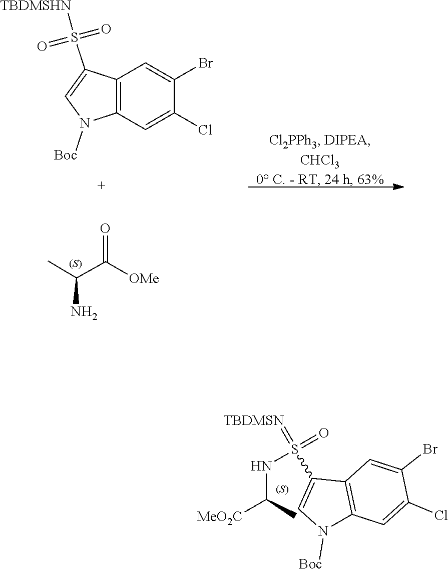

To a stirred solution of PPh₃Cl₂ (953 mg, 2.86 mmol, 1.5 eq) in CHCl₃ (20 mL) at 0° C., was added DIPEA (1.65 mL, 9.54 mmol, 5.0 eq), stirred for 1 h followed by addition of a solution of tert-butyl 5-bromo-3-(N-(tert-butyldimethylsilyl)sulfamoyl)-6-chloro-1H-indole-1-carboxylate (1 g, 1.91 mmol, 1.0 eq) in CHCl₃ (10 mL), continued stirring for 1 h at 0° C. Later, a solution of methyl L-alanine (801 mg, 5.72 mmol, 3.0 eq) in CHCl₃ (10 mL) was added at 0° C. and stirred for 24 h at room temperature. After completion of reaction by TLC, reaction mixture was quenched with ice cold water (30 mL) and extracted with EtOAc (2×30 mL). The combined organic layer was dried over Na₂SO₄, filtered, and concentrated under reduced pressure to afford crude product. The crude product was purified by silica gel (100-200) column chromatography [elution with 10% EtOAc in Hexane] to afford tert-butyl(S)-5-bromo-3-(N′-(tert-butyldimethylsilyl)-N-(1-methoxy-1-oxopropan-2-yl)sulfamidimidoyl)-6-chloro-1H-indole-1-carboxylate as a gummy liquid (700 mg, Yield: 63%).
TLC system: EtOAc/Hexane (10:90)
R_{f} value: ˜0.4

LCMS (m/z): 608.2 (M+H)⁺

### Synthesis of(S)-1-(5-bromo-6-chloro-1H-indol-3-yl)-4-methyl-4,5-dihydro-3H-116,2,5-thiadiazol-3-one 1-oxide

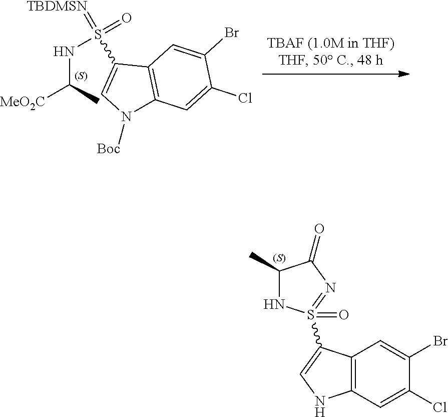

To a stirred solution of tert-butyl(S)-5-bromo-3-(N′-(tert-butyldimethylsilyl)-N-(1-methoxy-1-oxopropan-2-yl)sulfamidimidoyl)-6-chloro-1H-indole-1-carboxylate (450 mg, 0.74 mmol, 1.0 eq) in THF (4.5 mL) at 0° C., was added TBAF (1.0 M in THF) (4.5 mL, 10 Vol) and stirred at 50° C. for 48 h. After observing conversion by TLC, reaction mixture was quenched with NH₄Cl solution (20 mL) and extracted with DCM (3× 20 mL). The combined organic layer was dried over Na₂SO₄, filtered, and concentrated under reduced pressure to afford crude product. The crude product was purified by silica gel (100-200) column chromatography [elution with 5-7% MeOH in DCM] to afford (S)-1-(5-bromo-6-chloro-1H-indol-3-yl)-4-methyl-4,5-dihydro-3H-116,2,5-thiadiazol-3-one 1-oxide as an off white solid (350 mg, semi-pure).
TLC system: MeOH/DCM (10:90)
R_{f} value: ˜0.2

LCMS (m/z): 364.0 (M+H)⁺

¹H NMR (400 MHz, CDCl₃) δ: 8.30 (s, 1H), 7.87 (d, J=2.8 Hz, 1H), 7.79-7.74 (m, 1H), 4.33-4.28 (m, 1H), methyl protons merged with impurity. Excess tetra butyl ammonium impurities observed by NMR. This material was taken forward to the next step without purification.

### Synthesis of(S)-1-(6-chloro-5-(4-(1-hydroxycyclobutyl)phenyl)-1H-indol-3-yl)-4-methyl-4,5-dihydro-3H-116,2,5-thiadiazol-3-one 1-oxide

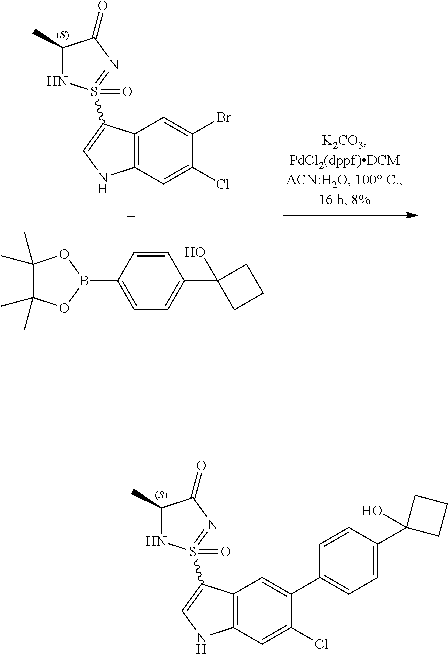

In a sealed tube, to a degassed solution of(S)-1-(5-bromo-6-chloro-1H-indol-3-yl)-4-methyl-4,5-dihydro-3H-116,2,5-thiadiazol-3-one 1-oxide (150 mg, 0.41 mmol, 1.0 eq) and 1-(4-(4,4,5,5-tetramethyl-1,3,2-dioxaborolan-2-yl)phenyl)cyclobutan-1-ol (181 mg, 0.66 mmol, 1.6 eq) in ACN:H₂O (1:1, 3 mL) at room temperature, was added K₂CO₃ (115 mg, 0.83 mmol, 2.0 eq) followed by PdCl₂(dppf).DCM (33 mg, 0.04 mmol, 0.1 eq) and stirred at 100° C. for 16 h. After completion of reaction by TLC, reaction mixture was diluted with water (10 mL) and extracted with 10% MeOH in DCM (2×20 mL). The combined organic layer was dried over Na₂SO₄, filtered, and concentrated under reduced pressure to afford crude product. The crude product was triturated with n-Pentane (10 mL), dried under vacuum, and purified by Grace RP column and prep-HPLC. Collected fractions were lyophilized to afford (S)-1-(6-chloro-5-(4-(1-hydroxycyclobutyl)phenyl)-1H-indol-3-yl)-4-methyl-4,5-dihydro-3H-116,2,5-thiadiazol-3-one 1-oxide as white solid (15 mg, Yield: 8%).
TLC system: MeOH/DCM (10:90)
R_{f} value: ˜0.3

LCMS (m/z): 428.1 (M+H)⁺

¹H NMR (400 MHz, DMSO-d₆) δ: 12.5 (br, 1H), 9.15 (br, 1H), 8.29 (d, J=2.8 Hz, 1H), 7.76 (d, J=2.0 Hz, 1H), 7.58 (d, J=8.0 Hz, 2H), 7.43-7.35 (m, 3H), 5.54 (s, 1H), 4.29-4.20 (m, 1H), 2.49-2.42 (m, 2H), 2.33-2.26 (m, 2H), 2.00-1.90 (m, 1H), 1.73-1.68 (m, 1H), 1.36-1.30 (2 d, 3H).

### (1S,4S)-1-(6-chloro-5-(4-(1-hydroxycyclobutyl)phenyl)-1H-indol-3-yl)-4-methyl-4,5-dihydro-3H-116,2,5-thiadiazol-3-one 1-oxide and

### (1R,4S)-1-(6-chloro-5-(4-(1-hydroxycyclobutyl)phenyl)-1H-indol-3-yl)-4-methyl-4,5-dihydro-3H-116,2,5-thiadiazol-3-one 1-oxide

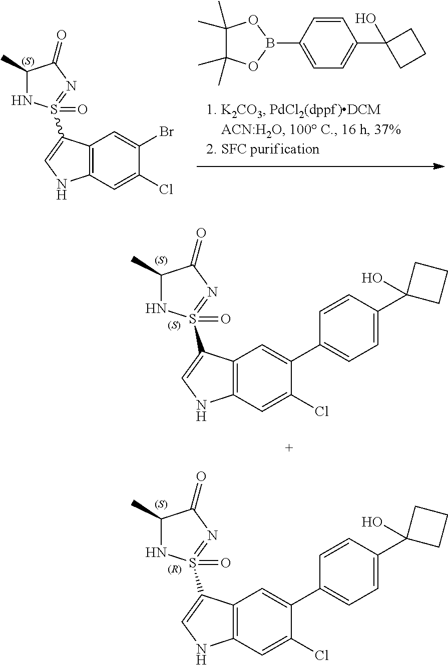

In a sealed tube, to a degassed solution of(S)-1-(5-bromo-6-chloro-1H-indol-3-yl)-4-methyl-4,5-dihydro-3H-116,2,5-thiadiazol-3-one 1-oxide (230 mg, 0.63 mmol, 1.0 eq) and 1-(4-(4,4,5,5-tetramethyl-1,3,2-dioxaborolan-2-yl)phenyl)cyclobutan-1-ol (278 mg, 1.01 mmol, 1.6 eq) in ACN:H₂O (1:1, 3 mL) at room temperature, was added K₂CO₃ (174 mg, 1.26 mmol, 2.0 eq) followed by PdCl₂(dppf)·DCM (51 mg, 0.06 mmol, 0.1 eq) and stirred at 100° C. for 16 h. After completion of reaction by TLC, reaction mixture was diluted with water (10 mL) and extracted with 10% MeOH in DCM (2×50 mL). The combined organic layer was dried over Na₂SO₄, filtered and concentrated under reduced pressure to afford crude product. The Crude product was purified by reverse phase column chromatography [elution with 40% ACN with 0.1% FA in H₂O] and pure fractions were lyophilized to afford (S)-1-(6-chloro-5-(4-(1-hydroxycyclobutyl)phenyl)-1H-indol-3-yl)-4-methyl-4,5-dihydro-3H-116,2,5-thiadiazol-3-one 1-oxide as a white solid (100 mg, Yield: 37%). TLC system: MeOH/DCM (10:90), R_{f} value: ˜0.3.

(S)-1-(6-chloro-5-(4-(1-hydroxycyclobutyl)phenyl)-1H-indol-3-yl)-4-methyl-4,5-dihydro-3H-116,2,5-thiadiazol-3-one 1-oxide (100 mg, Pure) was purified by SFC and recovered 41.5 mg of Peak 1 & 15.2 mg of Peak 2

Peak 1: LCMS (m/z): 430.2 (M+H)⁺; ¹H NMR (400 MHz, DMSO-d₆) δ: 12.57 (br, 1H), 9.22 (br, 1H), 8.29 (s, 1H), 7.76 (s, 1H), 7.57 (d, J=8.4 Hz, 2H), 7.38 (d, J=8.4 Hz, 2H), 7.35 (s, 1H), 5.54 (s, 1H), 4.24-4.20 (m, 1H), 2.49-2.43 (m, 2H), 2.33-2.28 (m, 2H), 1.96-1.93 (m, 1H), 1.68-1.66 (m, 1H), 1.39 (d, J=6.4 Hz, 3H).

Peak 2: LCMS (m/z): 428 (M−H)⁻; ¹H NMR (400 MHz, DMSO-d₆) δ: 8.27 (s, 1H), 7.76 (s, 1H), 7.58 (d, J=8.4 Hz, 2H), 7.43 (s, 1H), 7.36 (d, J=8.4 Hz, 2H), 5.54 (s, 1H), 4.28-4.23 (m, 1H), 2.50-2.43 (m, 2H), 2.33-2.26 (m, 2H), 1.96-1.94 (m, 1H), 1.73-1.67 (m, 1H), 1.34-1.32 (d, J=6.4 Hz, 3H). Exchangeable protons were not shown in the NMR

### SYNTHETIC EXAMPLE 3

### Synthesis of 3-(6-chloro-5-(4-(1-hydroxycyclobutyl)phenyl)-1H-pyrrolo [2,3-b]pyridin-3-yl)-1,2,4-oxadiazol-5(4H)-one

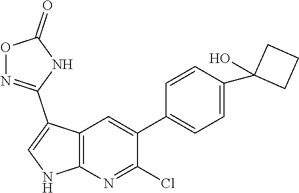 [SMILES: O=c1[nH]c(-c2c[nH]c3nc(Cl)c(-c4ccc(C5(O)CCC5)cc4)cc23)no1]

### Synthesis of 5-bromo-6-chloro-1H-pyrrolo[2,3-b]pyridine-3-carbaldehyde

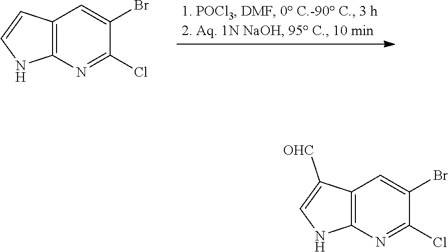 [SMILES: Clc1nc2[nH]ccc2cc1Br ; O=Cc1c[nH]c2nc(Cl)c(Br)cc12]

A solution of POCl₃ (4.03 mL, 43.3 mmol, 2.0 eq) and DMF (3.5 mL, 45.4 mmol, 2.1 eq) was stirred at 0° C., later added 5-bromo-6-chloro-1H-pyrrolo[2,3-b]pyridine (5 g, 21.6 mmol, 1.0 eq) in DMF (5 mL) at room temperature. The reaction mixture was stirred at 90° C. for 3 h. After completion of reaction by TLC, the reaction mixture was treated with 1N NaOH solution (10 mL) and stirred at 95° C. for 10 min, precipitated solid was filtered, washed with diethyl ether (50 mL) and dried under vacuum to afford 5-bromo-6-chloro-1H-pyrrolo[2,3-b]pyridine-3-carbaldehyde as pink solid (5.6 g, quantitative).
TLC system: EtOAc/Hexane (50:50)
R_{f} value: ˜0.25

LCMS (m/z): 259.1 (M+H)⁺

### Synthesis of 5-bromo-6-chloro-1H-pyrrolo[2,3-b]pyridine-3-carbonitrile

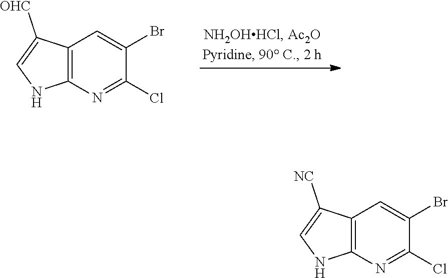 [SMILES: O=Cc1c[nH]c2nc(Cl)c(Br)cc12 ; N#Cc1c[nH]c2nc(Cl)c(Br)cc12]

To a stirred solution of 5-bromo-6-chloro-1H-pyrrolo[2,3-b]pyridine-3-carbaldehyde (5.6 g, 21.6 mmol, 1.0 eq, Crude) in Pyridine (28 mL) at room temperature, was added Ac₂O (4.1 mL, 43.2 mmol, 2.0 eq) and NH₂OH·HCl (1.65 g, 23.8 mmol, 1.1 eq). The reaction mixture was stirred at 90° C. for 2 h. After completion of reaction by TLC, reaction mixture was quenched with 2 N HCl solution (20 mL) and extracted with EtOAc (2×70 mL). The combined organic layer was dried over Na₂SO₄, filtered, and concentrated under reduced pressure to afford crude product. The crude product was triturated with diethyl ether (50 mL), decanted, and dried under vacuum to afford 5-bromo-6-chloro-1H-pyrrolo[2,3-b]pyridine-3-carbonitrile as off white solid (6 g, Crude, 72% purity).
TLC system: EtOAc/Hexane (50:50)
R_{f} value: ˜0.35

LCMS (m/z): 256.1 (M+H)⁺

### Synthesis of (Z)-5-bromo-6-chloro-N′-hydroxy-1H-pyrrolo[2,3-b]pyridine-3-carboximidamide

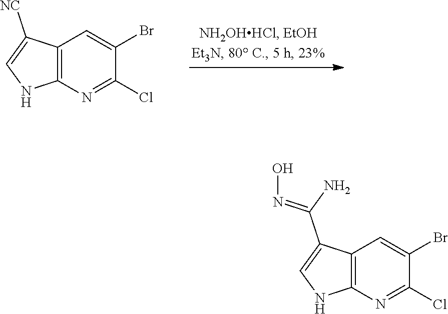 [SMILES: N#Cc1c[nH]c2nc(Cl)c(Br)cc12 ; N/C(=N\O)c1c[nH]c2nc(Cl)c(Br)cc12]

To a stirred solution of 5-bromo-6-chloro-1H-pyrrolo[2,3-b]pyridine-3-carbonitrile (3 g, 11.7 mmol, 1.0 eq) in EtOH (30 mL) at room temperature was added Et₃N (2.44 mL, 17.6 mmol, 1.5 eq) and NH₂OH·HCl (1.22 g, 17.6 mmol, 1.5 eq). The reaction mixture was stirred at 80° C. for 5 h. After completion of reaction by TLC, reaction mixture was diluted with water (30 mL) and extracted with EtOAc (3×75 mL). The combined organic layer was dried over Na₂SO₄, filtered, and concentrated under reduced pressure. Obtained crude was triturated with n-pentane, diethyl ether (50 mL), decanted and dried under vacuum to afford (Z)-5-bromo-6-chloro-N′-hydroxy-1H-pyrrolo[2,3-b]pyridine-3-carboximidamide as off white solid (1.4 g, yield: 23% in 2 steps).
TLC system: EtOAc/Hexane (70:30)

R_{f} value: ˜0.1; LCMS (m/z): 289.0 (M+H)⁺

### Synthesis of ethyl (Z)-5-bromo-6-chloro-3-(N′-((ethoxycarbonyl)oxy)carbamimidoyl)-1H-pyrrolo[2,3-b]pyridine-1-carboxylate

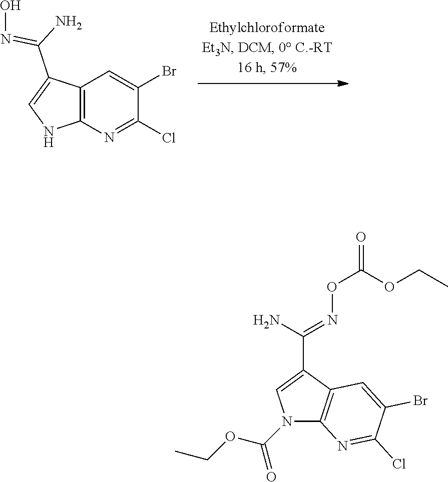 [SMILES: N/C(=N\O)c1c[nH]c2nc(Cl)c(Br)cc12 ; CCOC(=O)O/N=C(\N)c1cn(C(=O)OCC)c2nc(Cl)c(Br)cc12]

To a stirred solution of (Z)-5-bromo-6-chloro-N′-hydroxy-1H-pyrrolo[2,3-b] pyridine-3-carboximidamide (1.4 g, 4.84 mmol, 1.0 eq) in DCM (42 mL, 30 Vol) at 0° C., was added Et₃N (0.9 mL, 6.29 mmol, 1.3 eq) and Ethyl chloroformate (0.55 mL, 5.81 mmol, 1.2 eq). The reaction mixture was stirred at room temperature for 16 h. After completion of reaction by TLC, reaction mixture was diluted with water (50 mL) and extracted with EtOAc (3×50 mL). The organic layer was dried over Na₂SO₄, filtered, and concentrated. Obtained crude was triturated with n-pentane (20 mL), decanted, and dried under vacuum to afford ethyl (Z)-5-bromo-6-chloro-3-(N′-((ethoxycarbonyl)oxy) carbamimidoyl)-1H-pyrrolo[2,3-b]pyridine-1-carboxylate as off white solid (1.2 g, Yield: 57%).
TLC system: EtOAc/Hexane (50:50)
R_{f} value: ˜0.6

¹H NMR (400 MHz, CDCl₃) δ: 8.86 (s, 1H), 8.71 (s, 1H), 7.00 (s, 2H), 4.48 (q, J=7.2 Hz, 2H), 4.25 (q, J=7.2 Hz, 2H), 1.40 (t, J=7.2 Hz, 3H), 1.30 (t, J=7.2 Hz, 3H).

### Synthesis of 3-(5-bromo-6-chloro-1H-pyrrolo[2,3-b]pyridin-3-yl)-1,2,4-oxadiazol-5(4H)-one

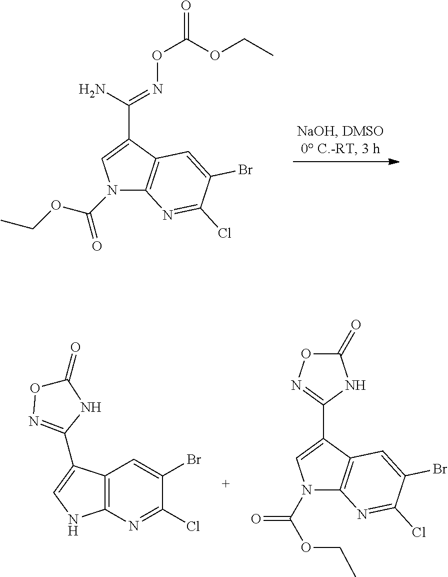 [SMILES: CCOC(=O)O/N=C(\N)c1cn(C(=O)OCC)c2nc(Cl)c(Br)cc12 ; O=c1[nH]c(-c2c[nH]c3nc(Cl)c(Br)cc23)no1 ; CCOC(=O)n1cc(-c2noc(=O)[nH]2)c2cc(Br)c(Cl)nc21]

To a stirred solution of ethyl (Z)-5-bromo-6-chloro-3-(N′-((ethoxycarbonyl)oxy)carbamimidoyl)-1H-pyrrolo[2,3-b]pyridine-1-carboxylate (1.2 g, 2.76 mmol, 1.0 eq) in DMSO (4.5 mL, 3.5 Vol) at 0° C., was added NaOH (133 mg, 3.31 mmol, 1.2 eq) and stirred for 3 h at room temperature. After completion of reaction by TLC, reaction mixture was diluted with water (50 mL), quenched with 2N HCl solution until reaching pH 2 and extracted with EtOAc (3×40 mL). The combined organic layer was dried over Na₂SO₄, filtered, and concentrated under reduced pressure. Obtained crude was purified by silica gel (100-200 mesh) column chromatography [elution with 65-70% EtOAc in Hexane] to afford 3-(5-bromo-6-chloro-1H-pyrrolo[2,3-b]pyridin-3-yl)-1,2,4-oxadiazol-5(4H)-one as yellow solid (0.5 g, mixture).
TLC system: EtOAc/Hexane (50:50)
R_{f} value: ˜0.2 & 0.4

LCMS (m/z): 315.0 & 387.0 (M+H)⁺

### Synthesis of 3-(6-chloro-5-(4-(1-hydroxycyclobutyl)phenyl)-1H-pyrrolo[2,3-b]pyridin-3-yl)-1,2,4-oxadiazol-5(4H)-one

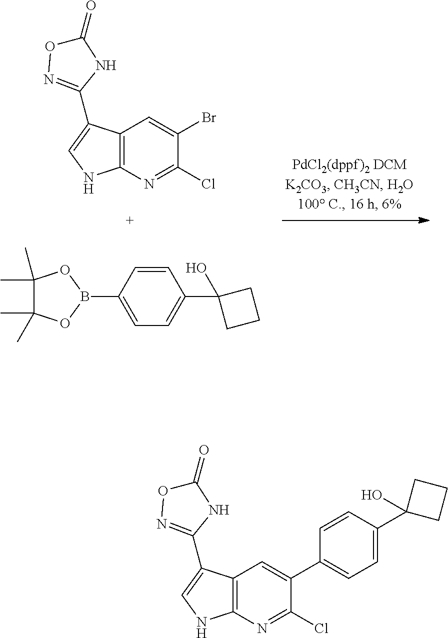 [SMILES: O=c1[nH]c(-c2c[nH]c3nc(Cl)c(Br)cc23)no1 ; CC1(C)OB(c2ccc(C3(O)CCC3)cc2)OC1(C)C ; O=c1[nH]c(-c2c[nH]c3nc(Cl)c(-c4ccc(C5(O)CCC5)cc4)cc23)no1]

In a sealed tube to a degassed solution of 3-(5-bromo-6-chloro-1H-pyrrolo[2,3-b]pyridin-3-yl)-1,2,4-oxadiazol-5(4H)-one (200 mg, 0.69 mmol, 1.0 eq (Mixture)) and 1-(4-(4,4,5,5-tetramethyl-1,3,2-dioxaborolan-2-yl)phenyl)cyclobutan-1-ol (276 mg, 1.10 mmol, 1.6 eq) in ACN:H₂O (1:1, 2 mL) at room temperature, was added K₂CO₃ (174 mg, 1.38 mmol, 2.0 eq) followed by Pd(dppf)Cl₂·DCM (46 mg, 0.069 mmol, 0.1 eq) and stirred at 100° C. for 16 h. After completion of reaction by TLC, reaction mixture was diluted with water (20 mL) and extracted with 10% MeOH in DCM (3× 20 mL). The combined organic layer was dried over Na₂SO₄, filtered, and concentrated under reduced pressure. Obtained crude was purified by reverse phase column chromatography [elution with 35% ACN in 0.1% FA in H₂O] followed by Prep-HPLC and lyophilized to afford 3-(6-chloro-5-(4-(1-hydroxycyclobutyl)phenyl)-1H-pyrrolo[2,3-b]pyridin-3-yl)-1,2,4-oxadiazol-5(4H)-one as off white solid (14 mg, Yield: 6%).
TLC system: MeOH/DCM (10:90), R_{f} value: ˜0.3;

LCMS (m/z): 381.1 (M−H)⁺;

¹H NMR (400 MHz, DMSO-d₆) δ: 12.79 (s, 2H), 8.17 (s, 1H), 8.14 (d, J=2.8 Hz, 1H), 7.60 (d, J=8.0 Hz, 2H), 7.47 (d, J=8.4 Hz, 2H), 5.57 (s, 1H), 2.47-2.42 (m, 2H), 2.35-2.27 (m, 2H), 2.00-1.90 (m, 1H), 1.75-1.71 (m, 1H).

### Prep Conditions:

Column Luna C₁₈ (250×19) 5 MICRON Buffer,
Mobile Phase A: 0.1% TFA in water; Mobile Phase B: ACN
Gradient (T/% B) 0.01/40, 13/40, 15/70, 16/95,
Flow Rate 15 mL/min Solubility ACN, WATER, THF

### SYNTHETIC EXAMPLE 4

### Synthesis of Compound I-7

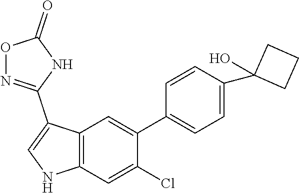 [SMILES: O=c1[nH]c(-c2c[nH]c3cc(Cl)c(-c4ccc(C5(O)CCC5)cc4)cc23)no1]

### Synthesis of 5-bromo-6-fluoro-1H-indole-3-carbaldehyde

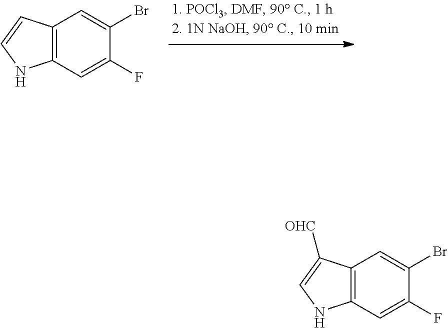 [SMILES: Fc1cc2[nH]ccc2cc1Br ; O=Cc1c[nH]c2cc(F)c(Br)cc12]

To a stirred solution of 5-bromo-6-fluoro-1H-indole (2 g, 9.34 mmol, 1.0 eq) in DMF (3.5 mL) at room temperature, was added POCl₃ (1.7 mL, 18.7 mmol, 2.0 eq). The reaction mixture was stirred at 90° C. for 1 h. After completion of reaction by TLC, the reaction mixture was treated with 1N NaOH solution (10 mL) and stirred at 90° C. for 10 min. The reaction mixture was diluted with H₂O (30 mL) and extracted with EtOAc (2×50 mL). The combined organic layer was dried over Na₂SO₄, filtered, and concentrated under reduced pressure to afford crude product. The crude product was triturated with n-pentane (30 mL), decanted, and dried under reduced pressure to afford 5-bromo-6-fluoro-1H-indole-3-carbaldehyde as a brown solid (1.9 g, Yield: 90%).
TLC system: EtOAc/Hexane (50:50)
R_{f} value: ˜0.3

LCMS (m/z): 242.31 (M+H)⁺; Bromo pattern observed.

### Synthesis of 5-bromo-6-fluoro-1H-indole-3-carbonitrile

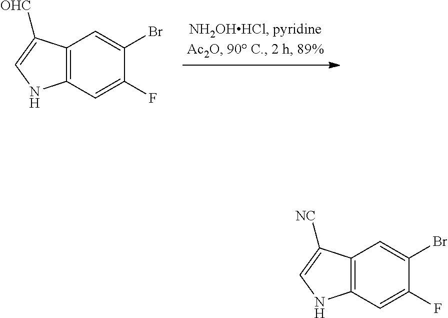 [SMILES: O=Cc1c[nH]c2cc(F)c(Br)cc12 ; N#Cc1c[nH]c2cc(F)c(Br)cc12]

To a stirred solution of 5-bromo-6-fluoro-1H-indole-3-carbaldehyde (1.9 g, 7.85 mmol, 1.0 eq) in pyridine (14.5 mL) at room temperature, was added Ac₂O (1.6 mL, 15.7 mmol, 2.0 eq) and NH₂OH·HCl (600 mg, 8.63 mmol, 1.1 eq). The reaction mixture was stirred at 90° C. for 2 h. After completion of reaction by TLC, the reaction mixture was quenched with 2 N HCl solution (20 mL) and extracted with EtOAc (2×50 mL). The combined organic layer was dried over Na₂SO₄, filtered, and concentrated under reduced pressure to afford crude product. The crude product was purified by silica gel (100-200 mesh) column chromatography [elution with 20% EtOAc in Hexane] to afford 5-bromo-6-fluoro-1H-indole-3-carbonitrile as a brown solid (1.6 g, Yield: 89%).
TLC system: EtOAc/Hexane (50:50)
R_{f} value: ˜0.6

LCMS (m/z): 239.43 (M+H)⁺

¹H NMR (400 MHz, CDCl₃) δ: 8.79 (br, 1H), 7.97 (d, J=6.4 Hz, 1H), 7.73 (d, J=3.2 Hz, 1H), 7.25 (s, 1H).

### Synthesis of 5-bromo-6-fluoro-1-((2-(trimethylsilyl)ethoxy)methyl)-1H-indole-3-carbonitrile

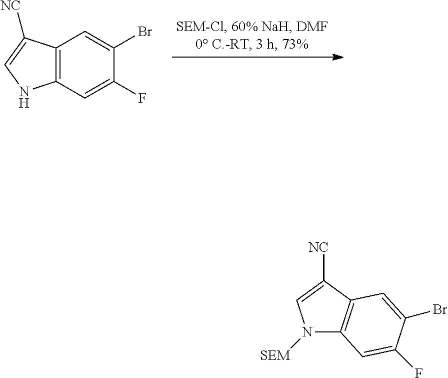 [SMILES: N#Cc1c[nH]c2cc(F)c(Br)cc12 ; C[Si](C)(C)CCOCn1cc(C#N)c2cc(Br)c(F)cc21]

To a stirred solution of 5-bromo-6-fluoro-1H-indole-3-carbonitrile (1.6 g, 6.69 mmol, 1.0 eq) in DMF (25.6 mL, 16 Vol) at 0° C., was added 60% NaH (323 mg, 13.4 mmol, 2.0 eq) and stirred for 30 min. After that SEM-Cl (1.4 mL, 8.03 mmol, 1.2 eq) was added to reaction mixture and stirred at room temperature for 3 h. After completion of reaction by TLC, the reaction mixture was diluted with ice cold water (30 mL) and extracted with EtOAc (2×30 mL). The combined organic layer was dried over Na₂SO₄, filtered, and concentrated under reduced pressure to afford crude product. The crude product was purified by silica gel (60-120 mesh) column chromatography [elution with 15% EtOAc in Hexane] to afford 5-bromo-6-fluoro-1-((2-(trimethylsilyl)ethoxy)methyl)-1H-indole-3-carbonitrile as a yellow solid (1.9 g, Yield: 73%).
TLC system: EtOAc/Hexane (50:50)
R_{f} value: ˜0.7

LCMS (m/z): 369.47 (M+H)⁺

¹H NMR (400 MHz, CDCl₃) δ: 7.94 (d, J=6.4 Hz, 1H), 7.67 (s, 1H), 7.34 (d, J=8.8 Hz, 1H), 5.44 (s, 2H), 3.47 (t, J=8.0 Hz, 2H), 0.90 (t, J=8.0 Hz, 2H), −0.04 (s, 9H).

### Synthesis of (E/Z)-5-bromo-6-fluoro-N′-hydroxy-1-((2-(trimethylsilyl)ethoxy)methyl)-1H-indole-3-carboximidamide

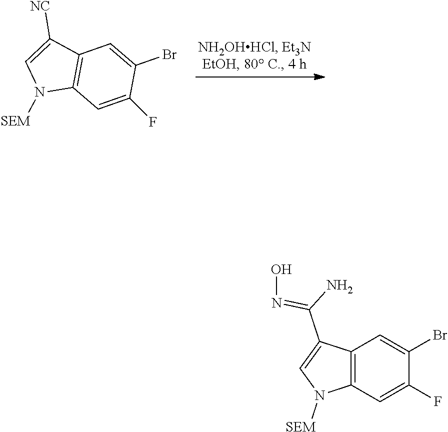 [SMILES: C[Si](C)(C)CCOCn1cc(C#N)c2cc(Br)c(F)cc21 ; C[Si](C)(C)CCOCn1cc(/C(N)=N/O)c2cc(Br)c(F)cc21]

To a stirred solution of 5-bromo-6-fluoro-1-((2-(trimethylsilyl)ethoxy)methyl)-1H-indole-3-carbonitrile (1.9 g, 5.15 mmol, 1.0 eq) in EtOH (19 mL) at room temperature, was added Et₃N (1.1 mL, 7.72 mmol, 1.5 eq) and NH₂OH·HCl (393 mg, 7.72 mmol, 1.5 eq). The reaction mixture was stirred at 80° C. for 4 h. After completion of reaction by TLC, the reaction mixture was diluted with water (35 mL) and extracted with EtOAc (2×35 mL). The combined organic layer was dried over Na₂SO₄, filtered, and concentrated under reduced pressure to afford crude product. The crude product was triturated with n-pentane (50 mL), decanted and dried under vacuum to afford (E/Z)-5-bromo-6-fluoro-N′-hydroxy-1-((2-(trimethylsilyl)ethoxy)methyl)-1H-indole-3-carboximidamide as brown solid (1.8 g, Crude).
TLC system: EtOAc/Hexane (50:50)
R_{f} value: ˜0.3

LCMS (m/z): 402.51 (M+H)⁺

### Synthesis of ethyl (E/Z)-((5-bromo-6-fluoro-1-((2-(trimethylsilyl)ethoxy)methyl)-1H-indol-3-yl)(hydroxyimino)methyl)carbamate

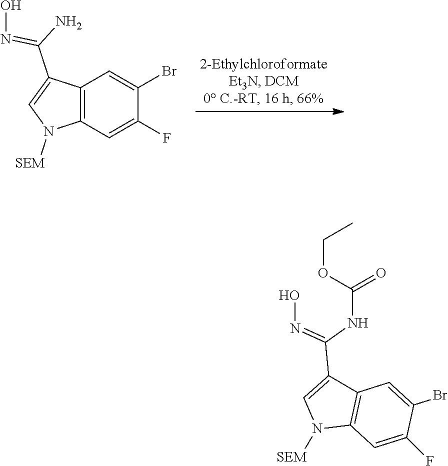 [SMILES: C[Si](C)(C)CCOCn1cc(/C(N)=N/O)c2cc(Br)c(F)cc21 ; CCOC(=O)N/C(=N\O)c1cn(COCC[Si](C)(C)C)c2cc(F)c(Br)cc12]

To a stirred solution of (E/Z)-5-bromo-6-fluoro-N′-hydroxy-1-((2-(trimethylsilyl)ethoxy)methyl)-1H-indole-3-carboximidamide (1.8 g, 4.48 mmol, 1.0 eq) in DCM (36 mL, 20 Vol) at 0° C., was added Et₃N (0.8 mL, 5.82 mmol, 1.3 eq) and 2-Ethylchloroformate (0.51 mL, 5.38 mmol, 1.2 eq). The reaction mixture was stirred at room temperature for 16 h. After completion of reaction by TLC, the reaction mixture was quenched with NaHCO₃ solution (30 mL) and extracted with EtOAc (2×30 mL). The organic layer was dried over Na₂SO₄, filtered, and concentrated to afford crude product. The crude product was purified by silica gel (100-200 mesh) column chromatography [elution with 20% EtOAc in Hexane] to afford ethyl (E/Z)-((5-bromo-6-fluoro-1-((2-(trimethylsilyl)ethoxy)methyl)-1H-indol-3-yl)(hydroxyimino)methyl)carbamate as brown solid (1.4 g, Yield: 66%).
TLC system: EtOAc/Hexane (50:50)
R_{f} value: ˜0.4

LCMS (m/z): 474.59 (M+H)⁺

¹H NMR (400 MHz, CDCl₃) δ: 8.26 (d, J=6.8 Hz, 1H), 7.54 (s, 1H), 7.27-7.25 (m, 1H), 5.39 (s, 2H), 5.06 (s, 1H), 4.35 (q, J=7.2 Hz, 2H), 3.45 (t, J=8.0 Hz, 2H), 1.40 (t, J=7.2 Hz, 2H), 0.88 (t, J=8.0 Hz, 2H), −0.05 (s, 9H)

### Synthesis of 3-(5-bromo-6-fluoro-1-((2-(trimethylsilyl)ethoxy)methyl)-1H-indol-3-yl)-1,2,4-oxadiazol-5(4H)-one

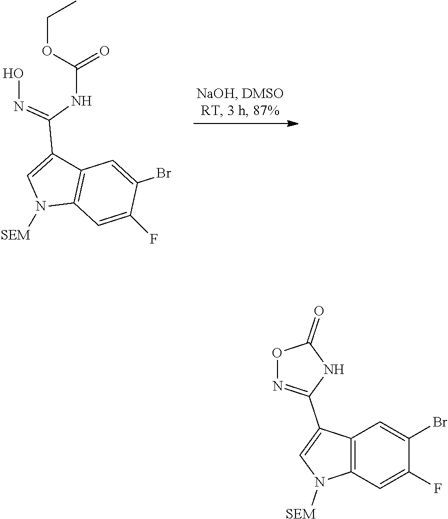 [SMILES: CCOC(=O)N/C(=N\O)c1cn(COCC[Si](C)(C)C)c2cc(F)c(Br)cc12 ; C[Si](C)(C)CCOCn1cc(-c2noc(=O)[nH]2)c2cc(Br)c(F)cc21]

To a stirred solution of ethyl (E/Z)-((5-bromo-6-fluoro-1-((2-(trimethylsilyl)ethoxy)methyl)-1H-indol-3-yl)(hydroxyimino)methyl)carbamate (1.4 g, 2.95 mmol, 1.0 eq) in DMSO (3.5 mL) at 0° C., was added NaOH (142 mg, 3.54 mmol, 1.2 eq) and stirred for 3 h at room temperature. After completion of reaction by TLC, the reaction mixture was diluted with water (30 mL), quenched with 2N HCl solution (5 mL) and extracted with EtOAc (3×30 mL). The combined organic layer was dried over Na₂SO₄, filtered, and concentrated under reduced pressure to afford crude product. The crude product was triturated with n-pentane (30 mL), decanted and dried under vacuum to afford 3-(5-bromo-6-fluoro-1-((2-(trimethylsilyl)ethoxy)methyl)-1H-indol-3-yl)-1,2,4-oxadiazol-5(4H)-one as brown solid (1.2 g, Yield: 87%).
TLC system: EtOAc/Hexane (50:50)
R_{f} value: ˜0.3

LCMS (m/z): 428.5 (M+H)⁺

¹H NMR (400 MHz, CDCl₃) δ: 11.90 (br, 1H), 8.28 (d, J=6.4 Hz, 1H), 7.84 (s, 1H), 7.30 (d, J=8.8 Hz, 1H), 5.47 (s, 2H), 3.51 (t, J=8.0 Hz, 2H), 0.92 (t, J=8.0 Hz, 2H), −0.03 (s, 9H).

### Synthesis of 3-(5-bromo-6-fluoro-1H-indol-3-yl)-1,2,4-oxadiazol-5(4H)-one

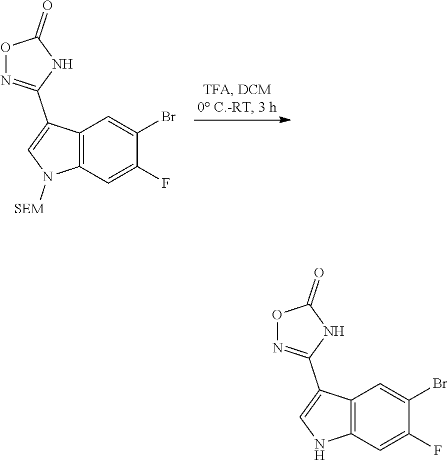 [SMILES: C[Si](C)(C)CCOCn1cc(-c2noc(=O)[nH]2)c2cc(Br)c(F)cc21 ; O=c1[nH]c(-c2c[nH]c3cc(F)c(Br)cc23)no1]

To a stirred solution of 3-(5-bromo-6-fluoro-1-((2-(trimethylsilyl)ethoxy)methyl)-1H-indol-3-yl)-1,2,4-oxadiazol-5(4H)-one (200 mg, 0.47 mmol, 1.0 eq) in DCM (2 mL) at 0° C., was added TFA (4 mL, 10 Vol) and stirred for 3 h at room temperature. After completion of reaction by TLC, the reaction mixture was filtered, washed with ether (10 mL) and collected solid was dried under vacuum to afford 3-(5-bromo-6-fluoro-1H-indol-3-yl)-1,2,4-oxadiazol-5(4H)-one as yellow solid (200 mg, crude).
TLC system: MeOH/DCM (10:90)
R_{f} value: ˜0.2

LCMS (m/z): 296.41 (M−H)⁻.

### Synthesis of 3-(6-fluoro-5-(4-(1-hydroxycyclobutyl)phenyl)-1H-indol-3-yl)-1,2,4-oxadiazol-5(4H)-one

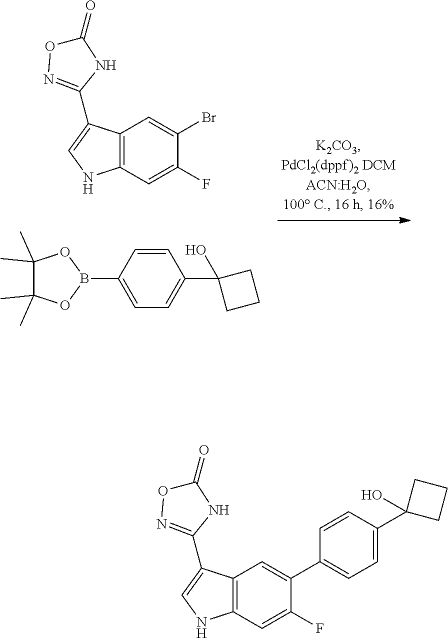 [SMILES: O=c1[nH]c(-c2c[nH]c3cc(F)c(Br)cc23)no1 ; CC1(C)OB(c2ccc(C3(O)CCC3)cc2)OC1(C)C ; O=c1[nH]c(-c2c[nH]c3cc(F)c(-c4ccc(C5(O)CCC5)cc4)cc23)no1]

In a sealed tube to a degassed solution of 3-(5-bromo-6-fluoro-1H-indol-3-yl)-1,2,4-oxadiazol-5(4H)-one (200 mg, 0.67 mmol, 1.0 eq) and 1-(4-(4,4,5,5-tetramethyl-1,3,2-dioxaborolan-2-yl)phenyl)cyclobutan-1-ol (300 mg, 1.07 mmol, 1.6 eq) in ACN:H₂O (1:1, 4 mL) at room temperature, was added K₂CO₃ (185 mg, 1.34 mmol, 2.0 eq) followed by Pd(dppf)Cl₂·DCM (54 mg, 0.067 mmol, 0.1 eq) and stirred at 100° C. for 16 h. After completion of reaction by TLC, the reaction mixture was diluted with water (20 mL) and extracted with EtOAc (2×20 mL). The combined organic layer was dried over Na₂SO₄, filtered, and concentrated under reduced pressure to afford crude product. The crude product was purified by reverse phase column chromatography [elution with 35% ACN in 0.1% FA in H₂O] to afford desired product. Obtained material was re-purified by reverse phase column chromatography [elution with 35% ACN in 0.1% FA in H₂O] and lyophilized to afford 3-(6-fluoro-5-(4-(1-hydroxycyclobutyl)phenyl)-1H-indol-3-yl)-1,2,4-oxadiazol-5(4H)-one as white solid (38 mg, Yield: 16%).
TLC system: MeOH/DCM (10:90)
R_{f} value: ˜0.3

LCMS (m/z): 364.0 (M−H)⁺

¹H NMR (400 MHz, DMSO-d₆) δ: 11.98 (s, 1H), 7.99 (d, J=2.8 Hz, 1H), 7.93 (d, J=8.0 Hz, 1H), 7.61-7.59 (m, 2H), 7.53-7.50 (m, 2H), 7.47 (d, J=11.2 Hz, 1H), 5.54 (s, 1H), 2.42-2.40 (m, 2H), 2.30-2.27 (m, 2H), 1.99-1.90 (m, 1H), 1.74-1.71 (m, 1H).

### SYNTHETIC EXAMPLE 5

### Synthesis of Compound I-8

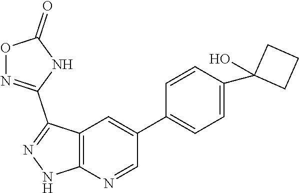 [SMILES: O=c1[nH]c(-c2n[nH]c3ncc(-c4ccc(C5(O)CCC5)cc4)cc23)no1]

### Synthesis of 5-bromo-3-iodo-1-((2-(trimethylsilyl)ethoxy)methyl)-1H-pyrazolo[3,4-b]pyridine

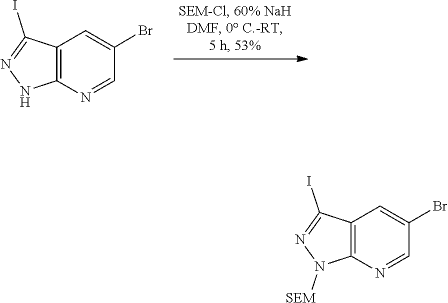 [SMILES: Brc1cnc2[nH]nc(I)c2c1 ; C[Si](C)(C)CCOCn1nc(I)c2cc(Br)cnc21]

To a stirred solution of 5-bromo-3-iodo-1H-pyrazolo[3,4-b]pyridine (5 g, 15.4 mmol, 1.0 eq) in DMF (50 mL, 10 Vol) at 0° C., was added 60% NaH (743 mg, 30.9 mmol, 2.0 eq) and stirred for 30 min. After that SEM-Cl (3.0 g, 18.5 mmol, 1.2 eq) was added to reaction mixture and stirred at room temperature for 5 h. After completion of reaction by TLC, the reaction mixture was diluted with ice cold water (100 mL) and extracted with DCM (2×200 mL). The combined organic layer was dried over Na₂SO₄, filtered, and concentrated under reduced pressure to afford crude product. The crude product was purified by silica gel (100-200 mesh) column chromatography [elution with 15-20% EtOAc in Hexane] to afford 5-bromo-3-iodo-1-((2-(trimethylsilyl)ethoxy)methyl)-1H-pyrazolo[3,4-b]pyridine as an off white solid (3.7 g, Yield: 53%).
TLC system: EtOAc/Hexane (20:80)
R_{f} value: ˜0.5

¹H NMR (400 MHz, CDCl₃) δ: 8.60 (d, J=2.0 Hz, 1H), 7.96 (d, J=2.4 Hz, 1H), 5.81 (s, 2H), 3.67-3.62 (m, 2H), 0.94-0.90 (m, 2H), −0.05 (s, 9H).

### Synthesis of 5-bromo-1-((2-(trimethylsilyl)ethoxy)methyl)-1H-pyrazolo[3,4-b]pyridine-3-carbonitrile

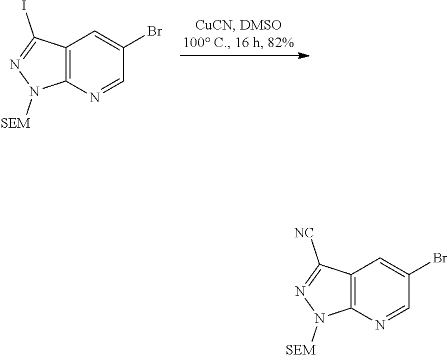 [SMILES: C[Si](C)(C)CCOCn1nc(I)c2cc(Br)cnc21 ; C[Si](C)(C)CCOCn1nc(C#N)c2cc(Br)cnc21]

To a stirred solution of 5-bromo-3-iodo-1-((2-(trimethylsilyl)ethoxy)methyl)-1H-pyrazolo[3,4-b]pyridine (2.8 g, 6.2 mmol, 1.0 eq) in DMSO (56 mL, 20 Vol) at room temperature, was added CuCN (1.1 g, 12.3 mmol, 2 eq) and stirred at 100° C. for 16 h. After completion of reaction by TLC, the reaction mixture was diluted with ice cold water (50 mL) and extracted with EtOAc (2×100 mL). The combined organic layer was dried over Na₂SO₄, filtered and concentrated under reduced pressure to afford crude product. The crude product was purified by silica gel (60-120 mesh) column chromatography [elution with 5% EtOAc in Hexane] to afford 5-bromo-1-((2-(trimethylsilyl)ethoxy)methyl)-1H-pyrazolo[3,4-b]pyridine-3-carbonitrile as white solid (1.8 g, Yield: 82%).
TLC system: EtOAc/Hexane (5:95)
R_{f} value: ˜0.3

LCMS: No proper ionization. Material was taken forward to next step.

### Synthesis of (E/Z)-5-bromo-N′-hydroxy-1-((2-(trimethylsilyl)ethoxy)methyl)-1H-pyrazolo[3,4-b]pyridine-3-carboximidamide

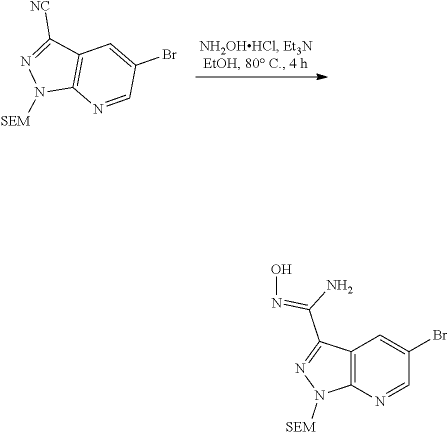 [SMILES: C[Si](C)(C)CCOCn1nc(C#N)c2cc(Br)cnc21 ; C[Si](C)(C)CCOCn1nc(/C(N)=N/O)c2cc(Br)cnc21]

To a stirred solution of 5-bromo-1-((2-(trimethylsilyl)ethoxy)methyl)-1H-pyrazolo[3,4-b]pyridine-3-carbonitrile (1.8 g, 5.1 mmol, 1.0 eq) in EtOH (18 mL, 10 Vol) at room temperature, was added Et₃N (1.1 mL, 7.6 mmol, 1.5 eq) and NH₂OH·HCl (390 mg, 5.6 mmol, 1.1 eq). The reaction mixture was stirred at 80° C. for 4 h. After completion of reaction by TLC, the reaction mixture was diluted with water (50 mL) and extracted with EtOAc (2×50 mL). The combined organic layer was dried over Na₂SO₄, filtered, and concentrated under reduced pressure to afford crude product. The crude product was triturated with n-pentane (30 mL), decanted and dried under vacuum to afford (E/Z)-5-bromo-N′-hydroxy-1-((2-(trimethylsilyl)ethoxy)methyl)-1H-pyrazolo[3,4-b]pyridine-3-carboximidamide as off white solid (1.7 g, Crude).
TLC system: EtOAc/Hexane (10:90)
R_{f} value: ˜0.2

LCMS (m/z): 386.4 (M+H)⁺

¹H NMR (400 MHz, CDCl₃) δ: 8.68 (s, 1H), 8.64 (s, 1H), 5.87 (m, 2H), 5.36 (s, 2H), 3.70-3.67 (m, 2H), 0.98 (t, J=8.0 Hz, 2H), −0.01 (s, 9H).

### Synthesis of (E/Z)-5-bromo-N′-((ethoxycarbonyl)oxy)-1-((2-(trimethylsilyl)ethoxy)methyl)-1H-pyrazolo[3,4-b]pyridine-3-carboximidamide

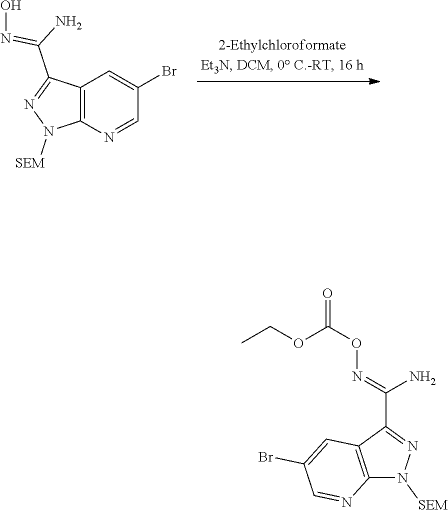 [SMILES: C[Si](C)(C)CCOCn1nc(/C(N)=N/O)c2cc(Br)cnc21 ; CCOC(=O)O/N=C(\N)c1nn(COCC[Si](C)(C)C)c2ncc(Br)cc12]

To a stirred solution of (E/Z)-5-bromo-N′-hydroxy-1-((2-(trimethylsilyl)ethoxy)methyl)-1H-pyrrolo[2,3-b]pyridine-3-carboximidamide (1.7 g, 4.4 mmol, 1.0 eq) in DCM (30 mL, 20 Vol) at 0° C., was added Et₃N (0.8 mL, 5.7 mmol, 1.3 eq) and 2-Ethylchloroformate (0.5 mL, 5.3 mmol, 1.2 eq). The reaction mixture was stirred at room temperature for 16 h. After completion of reaction by TLC, the reaction mixture was diluted with water (75 mL) and extracted with DCM (3×50 mL). The organic layer was dried over Na₂SO₄, filtered, and concentrated to afford crude product. The crude product was triturated with n-pentane (20 mL), decanted and dried under vacuum to afford (E/Z)-5-bromo-N′-((ethoxycarbonyl)oxy)-1-((2-(trimethylsilyl)ethoxy)methyl)-1H-pyrazolo[3,4-b]pyridine-3-carboximidamide as an off white solid (2 g, Crude).
TLC system: EtOAc/Hexane (30:70)
R_{f} value: ˜0.3

LCMS (m/z): 458.7 (M+H)⁺

¹H NMR (400 MHz, CDCl₃) δ: 8.79 (d, J=2.0 Hz, 1H), 8.61 (d, J=2.4 Hz, 1H), 5.84 (s, 2H), 5.56 (s, 2H), 4.37 (q, J=7.2 Hz, 2H), 3.62 (t, J=8.4 Hz, 2H), 1.41 (t, J=6.8 Hz, 3H), 0.91 (t, J=8.0 Hz, 2H), −0.06 (s, 9H).

### Synthesis of 3-(5-bromo-1-((2-(trimethylsilyl)ethoxy)methyl)-1H-pyrazolo[3,4-b]pyridin-3-yl)-1,2,4-oxadiazol-5(4H)-one

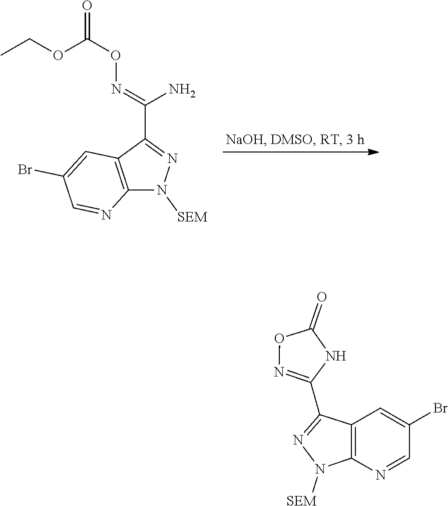

To a stirred solution of (E/Z)-5-bromo-N′-((ethoxycarbonyl)oxy)-1-((2-(trimethylsilyl)ethoxy)methyl)-1H-pyrazolo[3,4-b]pyridine-3-carboximidamide (2 g, 4.6 mmol, 1.0 eq) in DMSO (7 mL) at 0° C., was added NaOH (223 mg, 5.57 mmol, 1.2 eq) and stirred for 3 h at room temperature. After completion of reaction by TLC, the reaction mixture was diluted with water (30 mL), quenched with 2N HCl solution (8 mL) and extracted with 10% MeOH/DCM (3× 40 mL). The combined organic layer was dried over Na₂SO₄, filtered, and concentrated under reduced pressure to afford crude product. The crude product was triturated with n-pentane (30 mL), decanted and dried under vacuum to afford 3-(5-bromo-1-((2-(trimethylsilyl)ethoxy)methyl)-1H-pyrazolo[3,4-b]pyridin-3-yl)-1,2,4-oxadiazol-5(4H)-one as off white solid (1.5 g, Crude).
TLC system: EtOAc/Hexane (70:30)
R_{f} value: ˜0.1

LCMS (m/z): 410.3 (M−H)⁺

### Synthesis of 3-(5-bromo-1H-pyrazolo[3,4-b]pyridin-3-yl)-1,2,4-oxadiazol-5(4H)-one

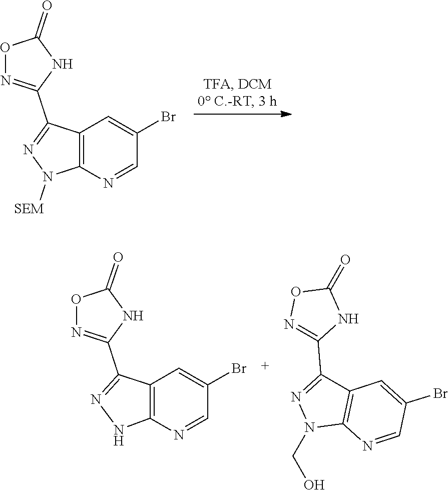

To a stirred solution of 3-(5-bromo-1-((2-(trimethylsilyl)ethoxy)methyl)-1H-pyrazolo[3,4-b]pyridin-3-yl)-1,2,4-oxadiazol-5(4H)-one (1 g, 2.3 mmol, 1.0 eq) in DCM (10 mL) at 0° C., was added TFA (10 mL, 10 Vol) and stirred for 3 h at room temperature. After completion of reaction by TLC, volatiles were removed, diluted with NaHCO₃ solution (35 mL) and extracted with 10% MeOH/DCM (2× 35 mL). The combined organic layer was dried over Na₂SO₄, filtered, and concentrated under reduced pressure to afford crude product. The crude product was triturated with n-pentane (20 mL), decanted, and dried under vacuum to afford 3-(5-bromo-1H-pyrazolo[3,4-b]pyridin-3-yl)-1,2,4-oxadiazol-5(4H)-one as off white solid (650 mg of crude).
TLC system: MeOH/DCM (10:90)
R_{f} value: ^{˜}0.1

LCMS (ESI, m/z): 282.0 (M−H)⁺ with 311.9 (M−H)⁺.

### Synthesis of 3-(5-(4-(1-hydroxycyclobutyl)phenyl)-1H-pyrazolo[3,4-b]pyridin-3-yl)-1,2,4-oxadiazol-5(4H)-one

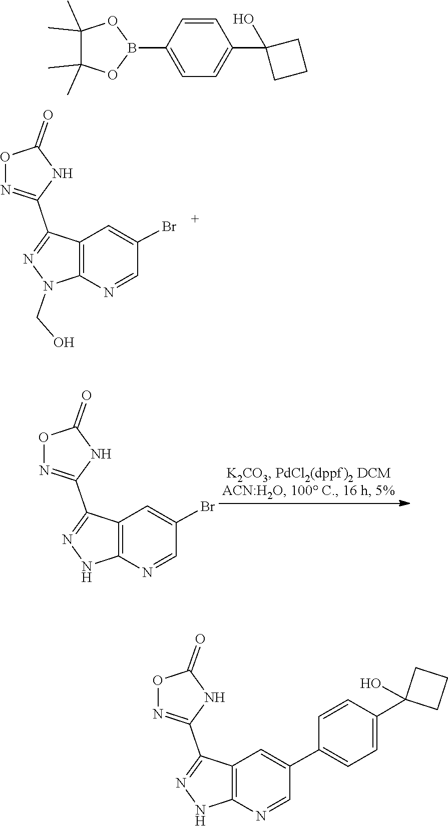

In a sealed tube to a degassed solution of 3-(5-bromo-1H-pyrazolo[3,4-b]pyridin-3-yl)-1,2,4-oxadiazol-5(4H)-one (300 mg, 1.0 mmol, 1.0 eq) and 1-(4-(4,4,5,5-tetramethyl-1,3,2-dioxaborolan-2-yl)phenyl)cyclobutan-1-ol (468 mg, 1.7 mmol, 1.6 eq) in ACN:H₂O (1:1, 6 mL) at room temperature, was added K₂CO₃ (294 mg, 2.1 mmol, 2.0 eq) followed by Pd(dppf)Cl₂·DCM (87 mg, 0.1 mmol, 0.1 eq) and stirred at 100° C. for 16 h. After completion of reaction by TLC, the reaction mixture was concentrated under reduced pressure and purified by reverse phase column chromatography [elution with 35-40% ACN in 0.1% FA in H₂O] to afford desired product along with impurity. This was dissolved in aq. NH₃ (0.5 mL) in dioxane (0.5 mL) and stirred for 16 h. Reaction mixture was concentrated under reduced pressure and purified by reverse phase column chromatography [elution with 35-40% ACN in 0.1% FA in H₂O] to afford 3-(5-(4-(1-hydroxycyclobutyl)phenyl)-1H-pyrrolo[2,3-b]pyridin-3-yl)-1,2,4-oxadiazol-5(4H)-one as off white solid (18 mg, Yield: ˜5%).
TLC system: MeOH/DCM (10:90)
R_{f} value: ˜0.1

LCMS (m/z): 348.0 [M−H]⁺

¹H NMR (400 MHz, DMSO-d₆) δ: 14.34 (s, 1H), 13.15 (s, 1H), 8.95 (d, J=2.0 Hz, 1H), 8.57 (d, J=2.0 Hz, 1H), 7.75 (d, J=8.4 Hz, 2H), 7.63 (d, J=8.4 Hz, 2H), 5.58 (s, 1H), 2.45-2.40 (m, 2H), 2.35-2.28 (m, 2H), 2.00-1.92 (m, 1H), 1.72-1.65 (m, 1H)

### SYNTHETIC EXAMPLE 6

### Synthesis of Compound I-9

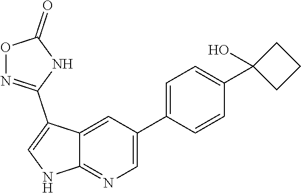 [SMILES: O=c1[nH]c(-c2c[nH]c3ncc(-c4ccc(C5(O)CCC5)cc4)cc23)no1]

### Synthesis of 5-bromo-3-iodo-1H-pyrrolo[2,3-b]pyridine

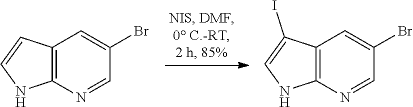 [SMILES: Brc1cnc2[nH]ccc2c1 ; Brc1cnc2[nH]cc(I)c2c1]

To a stirred solution of 5-bromo-1H-pyrrolo[2,3-b]pyridine (2 g, 10.2 mmol, 1.0 eq) in DMF (20 mL) at 0° C., was added N-iodosuccinimide (3.4 g, 15.2 mmol, 2.0 eq). The reaction mixture was stirred at room temperature for 2 h. After completion of reaction by TLC, reaction mixture was diluted with H₂O (75 mL) and extracted with EtOAc (3×75 mL). The combined organic layer was dried over Na₂SO₄, filtered, and concentrated under reduced pressure. Obtained crude product was triturated with n-pentane (30 mL), decanted and dried under vacuum to afford 5-bromo-3-iodo-1H-pyrrolo[2,3-b]pyridine as brown solid (2.8 g, Yield: 85%).
TLC system: EtOAc/Hexane (20:80)
R_{f} value: ˜0.6

¹H NMR (400 MHz, CDCl₃) δ: 9.40 (br s, 1H), 8.35 (d, J=2.0 Hz, 1H), 7.92 (d, J=1.6 Hz, 1H), 7.43 (d, J=2.4 Hz, 1H).

### Synthesis of 5-bromo-3-iodo-1-((2-(trimethylsilyl)ethoxy)methyl)-1H-pyrrolo[2,3-b]pyridine

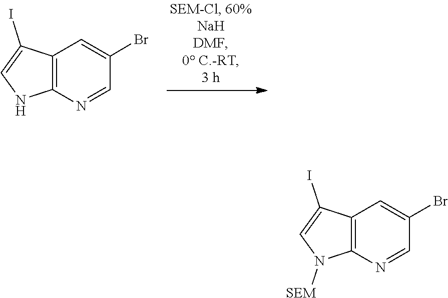 [SMILES: Brc1cnc2[nH]cc(I)c2c1 ; C[Si](C)(C)CCOCn1cc(I)c2cc(Br)cnc21]

To a stirred solution of 5-bromo-3-iodo-1H-pyrrolo[2,3-b]pyridine (2.8 g, 8.72 mmol, 1.0 eq) in DMF (28 mL, 10 Vol) at 0° C., was added 60% NaH (418 mg, 17.4 mmol, 2.0 eq) and stirred for 30 min. After that SEM-Cl (1.8 mL, 10.5 mmol, 1.2 eq) was added to reaction mixture and stirred at room temperature for 3 h. After completion of reaction by TLC, reaction mixture was diluted with ice cold water (30 mL) and extracted with EtOAc (3×50 mL). The combined organic layer was dried over Na₂SO₄, filtered and concentrated under reduced pressure to afford 5-bromo-3-iodo-1-((2-(trimethylsilyl)ethoxy)methyl)-1H-pyrrolo[2,3-b]pyridine as brown gummy liquid (3.7 g, Crude).
TLC system: EtOAc/Hexane (20:80),
R_{f} value: ˜0.7;

LCMS (m/z): 453.37 (M+H)⁺;

¹H NMR (400 MHz, CDCl₃) δ: 8.35 (d, J=2.0 Hz, 1H), 7.86 (d, J=2.0 Hz, 1H), 7.46 (s, 1H), 5.61 (s, 2H), 3.54-3.50 (m, 2H), 0.98-0.88 (m, 2H), −0.06 (s, 9H).

### Synthesis of 5-bromo-1-((2-(trimethylsilyl)ethoxy)methyl)-1H-pyrrolo[2,3-b]pyridine-3-carbonitrile

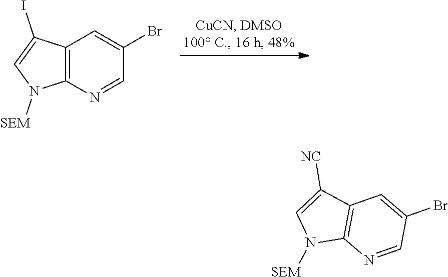 [SMILES: C[Si](C)(C)CCOCn1cc(I)c2cc(Br)cnc21 ; C[Si](C)(C)CCOCn1cc(C#N)c2cc(Br)cnc21]

To a stirred solution of 5-bromo-3-iodo-1-((2-(trimethylsilyl)ethoxy)methyl)-1H-pyrrolo[2,3-b]pyridine (3.7 g, 8.22 mmol, 1.0 eq) in DMSO (75 mL, 20 Vol) at room temperature was added CuCN (1.47 g, 16.4 mmol, 2 eq) and stirred at 100° C. for 16 h. Reaction mixture was diluted with ice cold water (70 mL) and extracted with EtOAc (2×70 mL). The combined organic layer was dried over Na₂SO₄, filtered and concentrated under reduced pressure. Obtained crude product was purified by silica gel (100-200 mesh) column chromatography [elution with 15-20% EtOAc in Hexane] to afford 5-bromo-1-((2-(trimethylsilyl)ethoxy)methyl)-1H-pyrrolo[2,3-b]pyridine-3-carbonitrile as off white solid (1.1 g, Yield: 48%).
TLC system: EtOAc/Hexane (30:70),
R_{f} value: ˜0.3;

¹H NMR (400 MHz, CDCl₃) δ: 8.49 (d, J=2.4 Hz, 1H), 8.23 (d, J=2.0 Hz, 1H), 7.89 (s, 1H), 5.68 (s, 2H), 3.56 (t, J=8.4 Hz, 2H), 0.92 (t, J=8.4 Hz, 2H), −0.04 (s, 9H). (1 g S.M recovered)

### Synthesis of (E/Z)-5-bromo-N′-hydroxy-1-((2-(trimethylsilyl)ethoxy)methyl)-1H-pyrrolo[2,3-b]pyridine-3-carboximidamide

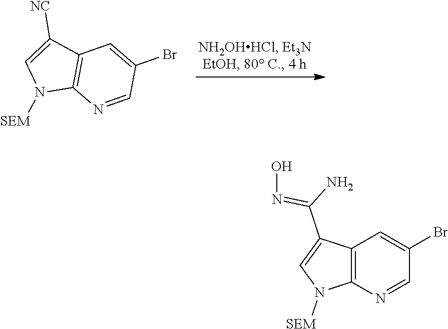 [SMILES: C[Si](C)(C)CCOCn1cc(C#N)c2cc(Br)cnc21 ; C[Si](C)(C)CCOCn1cc(/C(N)=N/O)c2cc(Br)cnc21]

To a stirred solution of 5-bromo-1-((2-(trimethylsilyl)ethoxy)methyl)-1H-pyrrolo[2,3-b]pyridine-3-carbonitrile (1.1 g, 3.13 mmol, 1.0 eq) in EtOH (11 mL) at room temperature was added Et₃N (0.7 mL, 4.70 mmol, 1.5 eq) and NH₂OH·HCl (285 mg, 4.07 mmol, 1.3 eq). The reaction mixture was stirred at 80° C. for 4 h. After completion of reaction by TLC, reaction mixture was diluted with water (50 mL) and extracted with EtOAc (2×50 mL). The combined organic layer was dried over Na₂SO₄, filtered and concentrated under reduced pressure. Obtained crude product was triturated with n-pentane (50 mL), decanted and dried under reduced pressure to afford (E/Z)-5-bromo-N′-hydroxy-1-((2-(trimethylsilyl)ethoxy)methyl)-1H-pyrrolo[2,3-b]pyridine-3-carboximidamide as an off white solid (1 g, semi-pure).
TLC system: EtOAc/Hexane (30:70),
R_{f} value: ˜0.2;

LCMS (m/z): 385.56 (M+H)⁺.

### Synthesis of (E/Z)-5-bromo-N′-((ethoxycarbonyl)oxy)-1-((2-(trimethylsilyl)ethoxy)methyl) 1H-pyrrolo[2,3-b]pyridine-3-carboximidamide

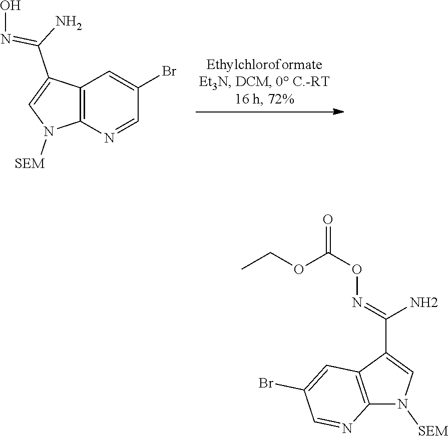 [SMILES: C[Si](C)(C)CCOCn1cc(/C(N)=N/O)c2cc(Br)cnc21 ; CCOC(=O)O/N=C(\N)c1cn(COCC[Si](C)(C)C)c2ncc(Br)cc12]

To a stirred solution of (E/Z)-5-bromo-N′-hydroxy-1-((2-(trimethylsilyl)ethoxy)methyl)-1H-pyrrolo[2,3-b]pyridine-3-carboximidamide (1 g, 2.60 mmol, 1.0 eq) in DCM (20 mL, 20 Vol) at 0° C., was added Et₃N (0.4 mL, 3.38 mmol, 1.3 eq) and 2-Ethylchloroformate (0.4 mL, 3.12 mmol, 1.2 eq). The reaction mixture was stirred at room temperature for 16 h. After completion of reaction by TLC, reaction mixture was diluted with water (30 mL) and extracted with DCM (3×50 mL). The combined organic layer was dried over Na₂SO₄, filtered and concentrated to afford crude product. The crude product was purified by silica gel (100-200 mesh) column chromatography [elution with 20-25% EtOAc in Hexane] to afford (E/Z)-5-bromo-N′-((ethoxycarbonyl)oxy)-1-((2-(trimethylsilyl)ethoxy)methyl)-1H-pyrrolo[2,3-b]pyridine-3-carboximidamide off white solid (0.85 g, Yield: 72%).
TLC system: EtOAc/Hexane (30:70),
R_{f} value: ˜0.5;

¹H NMR (400 MHz, CDCl₃) δ: 8.56 (d, J=2.0 Hz, 1H), 8.39 (d, J=2.0 Hz, 1H), 7.71 (s, 1H), 5.63 (s, 2H), 5.05 (s, 2H), 4.35 (q, J=7.2 Hz, 2H), 3.52 (t, J=8.4 Hz, 2H), 1.40 (t, J=7.2 Hz, 2H), 0.92 (t, J=8.4 Hz, 2H), −0.06 (s, 9H).

### Synthesis of 3-(5-bromo-1-((2-(trimethylsilyl)ethoxy)methyl)-1H-pyrrolo[2,3-b]pyridin-3-yl)-1,2,4-oxadiazol-5(4H)-one

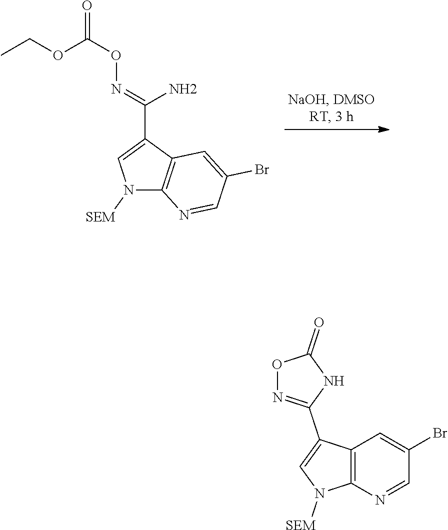 [SMILES: CCOC(=O)O/N=C(\N)c1cn(COCC[Si](C)(C)C)c2ncc(Br)cc12 ; C[Si](C)(C)CCOCn1cc(-c2noc(=O)[nH]2)c2cc(Br)cnc21]

To a stirred solution of (E/Z)-5-bromo-N′-((ethoxycarbonyl)oxy)-1-((2-(trimethylsilyl)ethoxy)methyl)-1H-pyrrolo[2,3-b]pyridine-3-carboximidamide (850 mg, 1.86 mmol, 1.0 eq) in DMSO (3 mL) at 0° C., was added NaOH (100 mg, 2.23 mmol, 1.2 eq) and stirred for 3 h at room temperature. After completion of reaction by TLC, the reaction mixture was diluted with water (30 mL), quenched with 2N HCl solution (5 mL) and extracted with 10% MeOH/DCM (3× 20 mL). The combined organic layer was dried over Na₂SO₄, filtered, and concentrated under reduced pressure to afford crude product. The crude product was triturated with n-pentane (30 mL), decanted and dried under vacuum to afford 3-(5-bromo-1-((2-(trimethylsilyl)ethoxy)methyl)-1H-pyrrolo[2,3-b]pyridin-3-yl)-1,2,4-oxadiazol-5(4H)-one as off white solid (900 mg, Crude).
TLC system: EtOAc/Hexane (50:50),
R_{f} value: ˜0.3;

LCMS (m/z): 411.71 (M+H)⁺;

### Synthesis of 3-(5-bromo-1H-pyrrolo[2,3-b]pyridin-3-yl)-1,2,4-oxadiazol-5(4H)-one

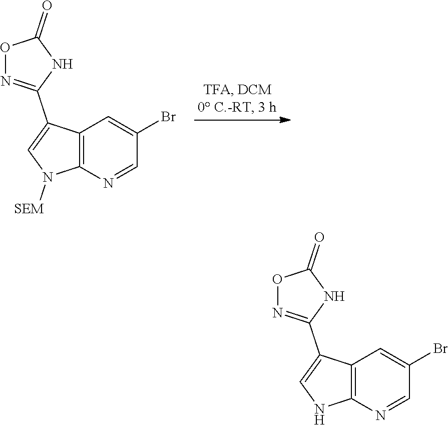 [SMILES: C[Si](C)(C)CCOCn1cc(-c2noc(=O)[nH]2)c2cc(Br)cnc21 ; O=c1[nH]c(-c2c[nH]c3ncc(Br)cc23)no1]

To a stirred solution of 3-(5-bromo-1-((2-(trimethylsilyl)ethoxy)methyl)-1H-pyrrolo[2,3-b]pyridin-3-yl)-1,2,4-oxadiazol-5(4H)-one (400 mg, 0.97 mmol, 1.0 eq) in DCM (4 mL) at 0° C., was added TFA (4 mL, 10 Vol) and stirred for 3 h at room temperature. After completion of reaction by TLC, volatiles were removed, diluted with sat. NaHCO₃ solution (30 mL) and extracted with 10% MeOH/DCM (3×20 mL). The combined organic layer was dried over Na₂SO₄, filtered and concentrated under reduced pressure to afford 3-(5-bromo-1H-pyrrolo[2,3-b]pyridin-3-yl)-1,2,4-oxadiazol-5(4H)-on as off white solid (250 mg of crude).
TLC system: MeOH/CH₂Cl₂ (10:90),
R_{f} value: ^{˜}0.2;

LCMS (ESI, m/z): 280.50 (M+H)⁺; material was used in next step without further purification.

### Synthesis of 3-(5-(4-(1-hydroxycyclobutyl)phenyl)-1H-pyrrolo[2,3-b]pyridin-3-yl)-1,2,4-oxadiazol-5(4H)-one

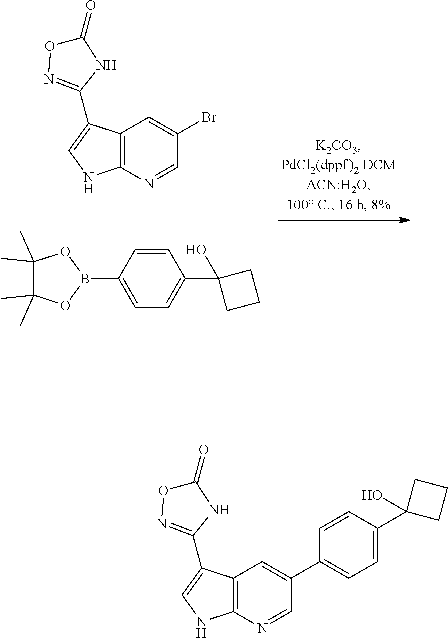 [SMILES: O=c1[nH]c(-c2c[nH]c3ncc(Br)cc23)no1 ; CC1(C)OB(c2ccc(C3(O)CCC3)cc2)OC1(C)C ; O=c1[nH]c(-c2c[nH]c3ncc(-c4ccc(C5(O)CCC5)cc4)cc23)no1]

In a sealed tube to a degassed solution of 3-(5-bromo-1H-pyrrolo[2,3-b]pyridin-3-yl)-1,2,4-oxadiazol-5(4H)-one (250 mg, 0.89 mmol, 1.0 eq) and 1-(4-(4,4,5,5-tetramethyl-1,3,2-dioxaborolan-2-yl)phenyl)cyclobutan-1-ol (391 mg, 1.42 mmol, 1.6 eq) in ACN:H₂O (1:1, 5 mL) at room temperature, was added K₂CO₃ (245 mg, 1.78 mmol, 2.0 eq) followed by Pd(dppf)Cl₂·DCM (72 mg, 0.089 mmol, 0.1 eq) and stirred at 100° C. for 16 h. After completion of reaction by TLC, reaction mixture was concentrated under reduced pressure to afford crude product. The crude product was purified by reverse phase column chromatography [elution with 35-40% ACN in 0.1% FA in H₂O] to afford 3-(5-(4-(1-hydroxycyclobutyl)phenyl)-1H-pyrrolo[2,3-b]pyridin-3-yl)-1,2,4-oxadiazol-5(4H)-one as off white solid (25 mg, Yield: 8%).
TLC system: MeOH/DCM (10:90),
R_{f} value: ˜0.3;

LCMS (m/z): 349.1 (M+H)⁺;

¹H NMR (400 MHz, DMSO-d₆) δ: 12.73 (br s, 1H), 12.61 (s, 1H), 8.69 (d, J=2.0 Hz, 1H), 8.41 (d, J=2.0 Hz, 1H), 8.12 (d, J=2.8 Hz, 1H), 7.70 (d, J=8.4 Hz, 2H), 7.62 (d, J=8.0 Hz, 2H), 5.55 (s, 1H), 2.45-2.40 (m, 2H), 2.34-2.27 (m, 2H), 1.98-1.90 (m, 1H), 1.68-1.62 (m, 1H).

### SYNTHETIC EXAMPLE 7

### Synthesis of Compound I-10

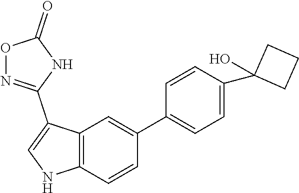 [SMILES: O=c1[nH]c(-c2c[nH]c3ccc(-c4ccc(C5(O)CCC5)cc4)cc23)no1]

### Synthesis of 5-bromo-1-((2-(trimethylsilyl)ethoxy)methyl)-1H-indole-3-carbonitrile

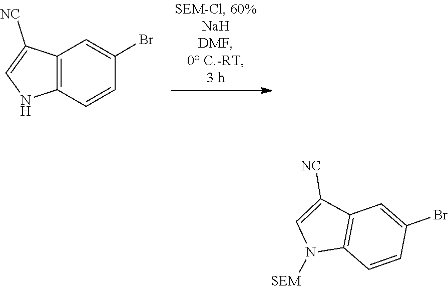 [SMILES: N#Cc1c[nH]c2ccc(Br)cc12 ; C[Si](C)(C)CCOCn1cc(C#N)c2cc(Br)ccc21]

To a stirred solution of 5-bromo-1H-indole-3-carbonitrile (2 g, 9.05 mmol, 1.0 eq) in DMF (20 mL, 20 Vol) at 0° C., was added 60% NaH (434 mg, 18.1 mmol, 2.0 eq) and stirred for 30 min. After that SEM-Cl (1.36 mL, 10.8 mmol, 1.2 eq) was added and stirred at room temperature for 3 h. After completion of reaction by TLC, reaction mixture was diluted with ice cold water (40 mL) and extracted with DCM (2×40 mL). The combined organic layer was washed with ice cold water (40 mL), dried over Na₂SO₄, filtered and concentrated under reduced pressure to afford 5-bromo-1-((2-(trimethylsilyl)ethoxy)methyl)-1H-indole-3-carbonitrile as brown solid (3.1 g, Crude).
TLC system: EtOAc/Hexane (30:70),
R_{f} value: ˜0.5;

¹H NMR (400 MHz, CDCl₃) δ: 7.91-7.90 (m, 1H), 7.67 (s, 1H), 7.46-7.44 (m, 2H), 5.48 (s, 2H), 3.47 (t, J=8.4 Hz, 2H), 0.90-0.87 (m, 2H), −0.04 (s, 9H).

### Synthesis of (Z)-5-bromo-N′-hydroxy-1-((2-(trimethylsilyl)ethoxy)methyl)-1H-indole-3-carboximidamide

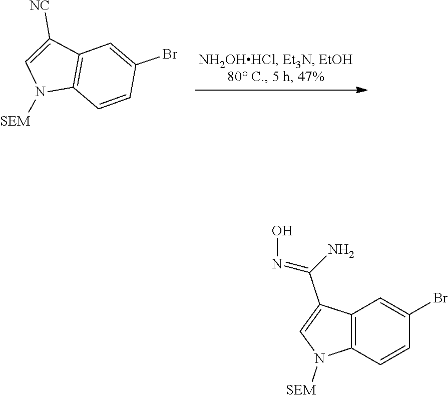 [SMILES: C[Si](C)(C)CCOCn1cc(C#N)c2cc(Br)ccc21 ; C[Si](C)(C)CCOCn1cc(/C(N)=N/O)c2cc(Br)ccc21]

To a stirred solution of 5-bromo-1-((2-(trimethylsilyl)ethoxy)methyl)-1H-indole-3-carbonitrile (3.1 g, 8.83 mmol, 1.0 eq) in EtOH (31 mL) at room temperature, was added Et₃N (1.9 mL, 13.2 mmol, 1.5 eq) and NH₂OH·HCl (910 mg, 13.2 mmol, 1.5 eq). The reaction mixture was stirred at 80° C. for 5 h. After completion of reaction by TLC, reaction mixture was diluted with water (60 mL) and extracted with EtOAc (2×60 mL). The combined organic layer was dried over Na₂SO₄, filtered, and concentrated under reduced pressure to. The crude product was triturated with n-pentane (50 mL), decanted and dried under reduced pressure to afford (Z)-5-bromo-N′-hydroxy-1-((2-(trimethylsilyl)ethoxy)methyl)-1H-indole-3-carboximidamide as a brown solid (1.6 g, Yield: 47%).
TLC system: EtOAc/Hexane (50:50),
R_{f} value: ˜0.4;

¹H NMR (400 MHz, CDCl₃) δ: 8.20 (s, 1H), 7.45 (s, 1H), 7.36 (s, 2H), 5.46 (s, 2H), 4.86 (s, 2H), 3.46 (t, J=8.0 Hz, 2H), 0.88 (t, J=8.0 Hz, 2H), −0.06 (s, 9H).

### Synthesis of (Z)-5-bromo-N′-((ethoxycarbonyl)oxy)-1-((2-(trimethylsilyl)ethoxy)methyl)-1H-indole-3-carboximidamide

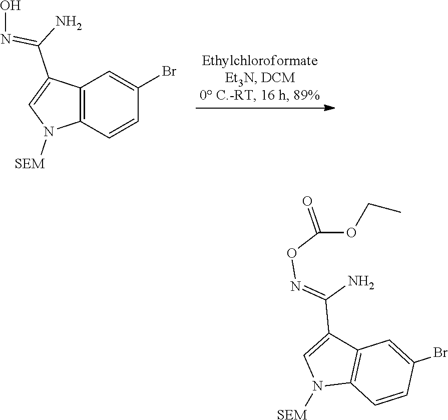 [SMILES: C[Si](C)(C)CCOCn1cc(/C(N)=N/O)c2cc(Br)ccc21 ; CCOC(=O)O/N=C(\N)c1cn(COCC[Si](C)(C)C)c2ccc(Br)cc12]

To a stirred solution of (Z)-5-bromo-N′-hydroxy-1-((2-(trimethylsilyl)ethoxy)methyl)-1H-indole-3-carboximidamide (1.6 g, 4.17 mmol, 1.0 eq) in DCM (32 mL, 20 Vol) at 0° C., was added Et₃N (0.8 mL, 5.42 mmol, 1.3 eq) and Ethyl chloroformate (0.5 mL, 5.00 mmol, 1.2 eq). The reaction mixture was stirred at room temperature for 16 h. After completion of reaction by TLC, reaction mixture was diluted with ice cold water (70 mL) and extracted with EtOAc (2×50 mL). The combined organic layer was dried over Na₂SO₄, filtered, and concentrated to afford crude product. The crude product was triturated with n-pentane (20 mL), decanted and dried under reduced pressure to afford (Z)-5-bromo-N′-((ethoxycarbonyl)oxy)-1-((2-(trimethylsilyl)ethoxy)methyl)-1H-indole-3-carboximidamide as an off white solid (1.7 g, Yield: 89%).
TLC system: EtOAc/Hexane (40:60),
R_{f} value: ˜0.5;

LCMS (m/z): 456.61 [M+H]⁺;

¹H NMR (400 MHz, CDCl₃) δ: 8.17 (s, 1H), 7.58 (s, 1H), 7.38 (s, 2H), 5.44 (s, 2H), 5.09 (s, 2H), 4.35 (q, J=6.8 Hz, 2H), 3.45 (t, J=8.4 Hz, 2H), 1.40 (t, J=7.2 Hz, 3H), 0.88 (t, J=8.4 Hz, 2H), −0.06 (s, 9H).

### Synthesis of 3-(5-bromo-1-((2-(trimethylsilyl)ethoxy)methyl)-1H-indol-3-yl)-1,2,4-oxadiazol-5(4H)-one

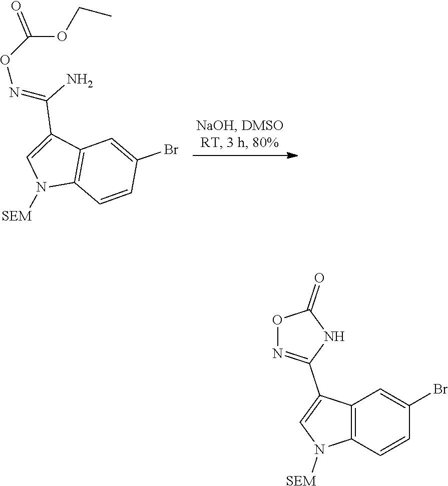 [SMILES: CCOC(=O)O/N=C(\N)c1cn(COCC[Si](C)(C)C)c2ccc(Br)cc12 ; C[Si](C)(C)CCOCn1cc(-c2noc(=O)[nH]2)c2cc(Br)ccc21]

To a stirred solution of (Z)-5-bromo-N′-((ethoxycarbonyl)oxy)-1-((2-(trimethylsilyl)ethoxy)methyl)-1H-indole-3-carboximidamide (1.7 g, 3.73 mmol, 1.0 eq) in DMSO (6 mL) at 0° C., was added NaOH (180 mg, 4.48 mmol, 1.2 eq) and stirred for 3 h at room temperature. After completion of reaction by TLC, the reaction mixture was diluted with water (25 mL), quenched with 2N HCl solution (5 mL) and extracted with EtOAc (3×50 mL). The combined organic layer was dried over Na₂SO₄, filtered, and concentrated under reduced pressure. The crude product was triturated with n-pentane (30 mL), decanted and dried under reduced pressure to afford 3-(5-bromo-1-((2-(trimethylsilyl)ethoxy)methyl)-1H-indol-3-yl)-1,2,4-oxadiazol-5(4H)-one as off white solid (1.2 g, Yield: 80%).
TLC system: EtOAc/Hexane (50:50),
R_{f} value: ˜0.3;

LCMS (m/z): 410.53 (M+H)⁺.

¹H NMR (400 MHz, CDCl₃) δ: 8.10 (s, 1H), 8.05 (s, 1H), 7.70 (d, J=8.8 Hz, 1H), 7.49 (dd, J=8.8 & 2.0 Hz, 1H), 5.66 (s, 2H), 3.49-3.45 (m, 2H), 0.82 (t, J=8.0 Hz, 2H), −0.10 (s, 9H).

### Synthesis of 3-(5-bromo-1H-indol-3-yl)-1,2,4-oxadiazol-5(4H)-one

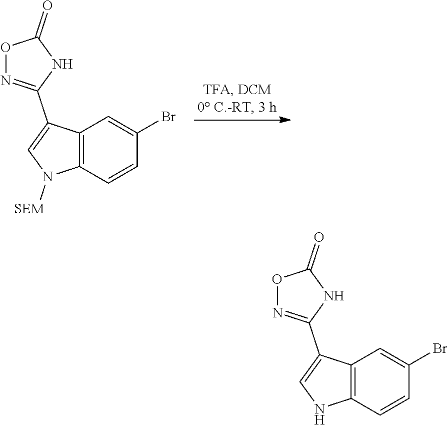 [SMILES: C[Si](C)(C)CCOCn1cc(-c2noc(=O)[nH]2)c2cc(Br)ccc21 ; O=c1[nH]c(-c2c[nH]c3ccc(Br)cc23)no1]

To a stirred solution of 3-(5-bromo-1-((2-(trimethylsilyl)ethoxy)methyl)-1H-indol-3-yl)-1,2,4-oxadiazol-5(4H)-one (400 mg, 0.97 mmol, 1.0 eq) in DCM (4 mL) at 0° C., was added TFA (4 mL, 10 Vol) and stirred for 3 h at room temperature. After completion of reaction by TLC, reaction mixture was concentrated, quenched with NaHCO₃ solution (20 mL) and extracted with 10% MeOH in DCM (2×20 mL). The combined organic layer was dried over Na₂SO₄, filtered and concentrated under reduced pressure to afford 3-(5-bromo-1H-indol-3-yl)-1,2,4-oxadiazol-5(4H)-one as off white solid (250 mg, crude).
TLC system: MeOH/DCM (10:90),
R_{f} value: ˜0.2;

LCMS (m/z): 277.9 (M−H)⁺.

### Synthesis of 3-(5-(4-(1-hydroxycyclobutyl)phenyl)-1H-indol-3-yl)-1,2,4-oxadiazol-5(4H)-one

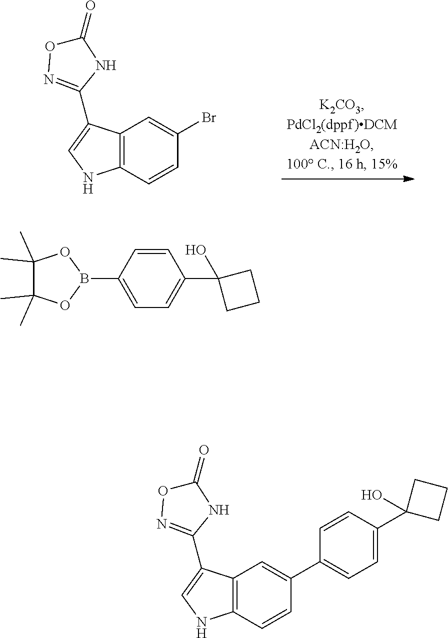 [SMILES: O=c1[nH]c(-c2c[nH]c3ccc(Br)cc23)no1 ; CC1(C)OB(c2ccc(C3(O)CCC3)cc2)OC1(C)C ; O=c1[nH]c(-c2c[nH]c3ccc(-c4ccc(C5(O)CCC5)cc4)cc23)no1]

In a sealed tube to a degassed solution of 3-(5-bromo-1H-indol-3-yl)-1,2,4-oxadiazol-5(4H)-one (200 mg, 0.71 mmol, 1.0 eq) and 1-(4-(4,4,5,5-tetramethyl-1,3,2-dioxaborolan-2-yl)phenyl)cyclobutan-1-ol (313 mg, 1.14 mmol, 1.6 eq) in ACN:H₂O (1:1, 4 mL) at room temperature, was added K₂CO₃ (197 mg, 1.42 mmol, 2.0 eq) followed by Pd(dppf)Cl₂·DCM (58 mg, 0.071 mmol, 0.1 eq) and stirred at 100° C. for 16 h. After completion of reaction by TLC, the reaction mixture was diluted with water (20 mL) and extracted with EtOAc (2×20 mL). The combined organic layer was dried over Na₂SO₄, filtered and concentrated under reduced pressure to afford crude product. The crude product was purified by silica gel (100-200 mesh) column chromatography [elution with 35% ACN in 0.1% FA in H₂O] and lyophilized to afford 3-(5-(4-(1-hydroxycyclobutyl)phenyl)-1H-indol-3-yl)-1,2,4-oxadiazol-5(4H)-one as off white solid (38 mg, Yield: 15%).
TLC system: MeOH/DCM (10:90),
R_{f} value: ˜0.4;

LCMS (m/z): 348.2 (M+H)⁺;

¹H NMR (400 MHz, DMSO-d₆) δ: 12.54 (s, 1H), 11.99 (s, 1H), 8.13 (s, 1H), 8.01 (d, J=2.8 Hz, 1H), 7.65-7.55 (m, 6H), 5.52 (s, 1H), 2.44-2.40 (m, 2H), 2.33-2.26 (m, 2H), 1.95-1.92 (m, 1H), 1.69-1.66 (m, 1H).

### SYNTHETIC EXAMPLE 8

### Synthesis of Compound I-11

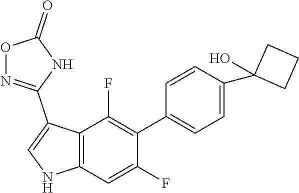 [SMILES: O=c1[nH]c(-c2c[nH]c3cc(F)c(-c4ccc(C5(O)CCC5)cc4)c(F)c23)no1]

### Synthesis of 4,6-difluoroindoline

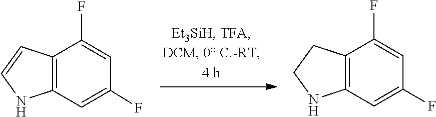 [SMILES: Fc1cc(F)c2cc[nH]c2c1 ; Fc1cc(F)c2c(c1)NCC2]

To a stirred solution of 4,6-difluoro-1H-indole (10 g, 64.5 mmol, 1.0 eq) in DCM (200 mL) at 0° C. was added triethyl silane (27 mL, 167.7 mmol, 2.6 eq), TFA (100 mL, 10 vol) and stirred at RT for 4 h. After completion of reaction by TLC, the reaction mixture was concentrated and quenched with aq. NaHCO₃ solution (200 mL) and extracted with DCM (2×100 mL). The combined organic layer was dried over Na₂SO₄, filtered, and concentrated under reduced pressure to afford crude product. The crude product was purified by silica gel (60-120 mesh) column chromatography [elution with 5-10% EtOAc in Hexane] to afford 4,6-difluoroindoline as a brown liquid (10 g, Crude).
TLC system: EtOAc/Hexane (15:85),
R_{f} value: ˜0.6;

¹H NMR (400 MHz, CDCl₃) δ: 6.14-6.09 (m, 2H), 3.63 (t, J=8.4 Hz, 2H), 3.01 (t, J=8.4 Hz, 2H). Et₃Si impurities observed.

### Synthesis of 5-bromo-4,6-difluoroindoline

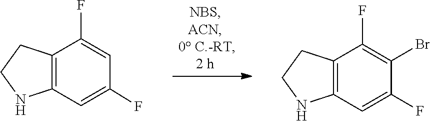 [SMILES: Fc1cc(F)c2c(c1)NCC2 ; Fc1cc2c(c(F)c1Br)CCN2]

To a stirred solution of 4,6-difluoroindoline (10 g, 64.5 mmol, 1.0 eq) in ACN (200 mL) at 0° C., was added NBS (8 g, 45.2 mmol, 0.7 eq) and allowed to stir at RT for 2 h. After completion of reaction by TLC, the reaction mixture was quenched with ice cold water (100 mL) and extracted with EtOAc (2×100 mL). The combined organic layer was dried over Na₂SO₄, filtered, and concentrated under reduced pressure to afford crude product. The crude product was purified by silica gel (230-400 mesh) column chromatography [elution with 10-15% EtOAc in Hexane] to afford 5-bromo-4,6-difluoroindoline as a pink solid (5.1 g, Crude).
TLC system: EtOAc/Hexane (20:80),
R_{f} value: ˜0.3;

¹H NMR (400 MHz, CDCl₃) δ: 6.23-6.20 (m, 1H), 3.66 (t, J=8.4 Hz, 2H), 3.07 (t, J=8.4 Hz, 2H). Et₃Si impurities carried from the previous reaction step.

### Synthesis of 5-bromo-4,6-difluoro-1H-indole

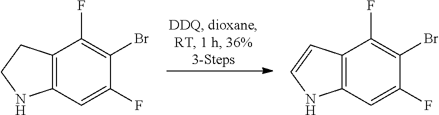 [SMILES: Fc1cc2c(c(F)c1Br)CCN2 ; Fc1cc2[nH]ccc2c(F)c1Br]

To a stirred solution of 5-bromo-4,6-difluoroindoline (5 g, 21.5 mmol, 1.0 eq) in 1,4-Dioxane (50 mL) at room temperature, was added DDQ (5.87 g, 25.8 mmol, 1.2 eq). The reaction mixture was stirred at room temperature for 1 h. After completion of reaction by TLC, reaction mixture was quenched with ice cold water (100 mL) and extracted with EtOAc (2×100 mL). The combined organic layer was dried over Na₂SO₄, filtered, and concentrated under reduced pressure to afford crude product. The crude product was purified by silica gel (100-200 mesh) column chromatography [elution with 10-15% EtOAc in Hexane] to afford 5-bromo-4,6-difluoro-1H-indole as a gummy solid (5.5 g, Yield: 36% in 3-steps).
TLC system: EtOAc/Hexane (20:80),
R_{f} value: ˜0.6;

¹H NMR (400 MHz, CDCl₃) δ: 8.33 (s, 1H), 7.19-7.17 (m, 1H), 7.03-7.01 (m, 1H), 6.62-6.60 (m, 1H).

### Synthesis of 5-bromo-4,6-difluoro-1H-indole-3-carbaldehyde

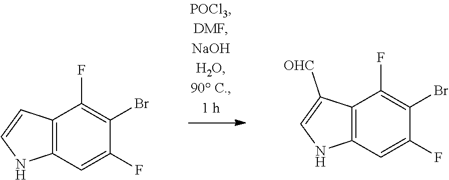 [SMILES: Fc1cc2[nH]ccc2c(F)c1Br ; O=Cc1c[nH]c2cc(F)c(Br)c(F)c12]

To a stirred solution of 5-bromo-4,6-difluoro-1H-indole (5.5 g, 23.7 mmol, 1.0 eq) in DMF (5.5 mL) at room temperature, was added POCl₃ (4.42 mL, 47.4 mmol, 2.0 eq) and heated at 90° C. for 1 h. After completion of reaction by TLC, reaction mixture was treated with 1N NaOH solution (15 mL) and stirred at 90° C. for 10 min. The reaction mixture was diluted with H₂O (50 mL) and extracted with EtOAc (2×100 mL). The combined organic layer was dried over Na₂SO₄, filtered, and concentrated under reduced pressure to afford crude product. The crude product was triturated with n-pentane (50 mL), decanted, and dried under vacuum to afford 5-bromo-4,6-difluoro-1H-indole-3-carbaldehyde as an off white solid (4.5 g, Crude).
TLC system: EtOAc/Hexane (50:50),
R_{f} value: ˜0.2;

LCMS (m/z): 257.0 (M+H+2)⁺.

### Synthesis of 5-bromo-4,6-difluoro-1H-indole-3-carbonitrile

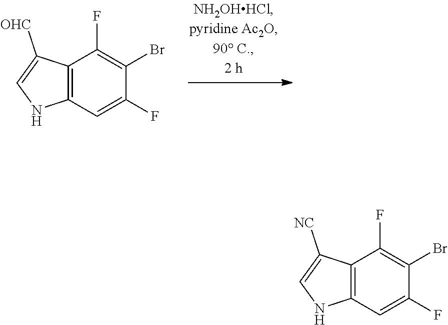 [SMILES: O=Cc1c[nH]c2cc(F)c(Br)c(F)c12 ; N#Cc1c[nH]c2cc(F)c(Br)c(F)c12]

To a stirred solution of 5-bromo-4,6-difluoro-1H-indole-3-carbaldehyde (5 g, 19.2 mmol, 1.0 eq) in pyridine (25 mL) at room temperature, was added Ac₂O (3.9 mL, 38.5 mmol, 2.0 eq) and NH₂OH·HCl (1.5 g, 21.2 mmol, 1.5 eq). The reaction mixture was stirred at 90° C. for 2 h. After completion of reaction by TLC, the reaction mixture was quenched with 2 N HCl solution (20 mL) and extracted with EtOAc (2×100 mL). The combined organic layer was dried over Na₂SO₄, filtered, and concentrated under reduced pressure to afford crude product. The crude product was triturated with n-pentane & diethyl ether (1:1) (20 mL) to afford 5-bromo-4,6-difluoro-1H-indole-3-carbonitrile as a brown solid (4.6 g, Crude).
TLC system: EtOAc/Hexane (50:50),
R_{f} value: ˜0.5;

LCMS (m/z): 257.03 (M+H)⁺.

### Synthesis of 5-bromo-4,6-difluoro-1-((2-(trimethylsilyl)ethoxy)methyl)-1H-indole-3-carbonitrile

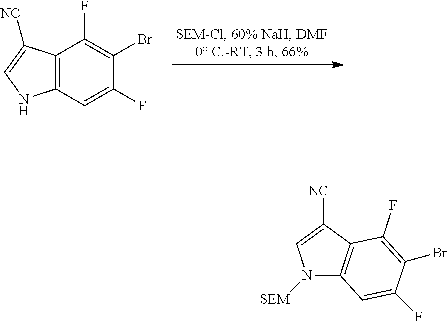 [SMILES: N#Cc1c[nH]c2cc(F)c(Br)c(F)c12 ; C[Si](C)(C)CCOCn1cc(C#N)c2c(F)c(Br)c(F)cc21]

To a stirred solution of 5-bromo-4,6-difluoro-1H-indole-3-carbonitrile (4.6 g, 17.9 mmol, 1.0 eq) in DMF (46 mL, 10 Vol) at 0° C., was added 60% NaH (0.86 g, 35.8 mmol, 2.0 eq) and stirred for 30 min. After that SEM-Cl (3.8 mL, 21.5 mmol, 1.2 eq) was added to reaction mixture and stirred at room temperature for 3 h. After completion of reaction by TLC, the reaction mixture was diluted with ice cold water (30 mL) and extracted with EtOAc (2×150 mL). The combined organic layer was dried over Na₂SO₄, filtered, and concentrated under reduced pressure to afford crude product. The crude product was purified by silica gel (60-120 mesh) column chromatography [elution with 10-5% EtOAc in Hexane] to afford 5-bromo-4,6-difluoro-1-((2-(trimethylsilyl)ethoxy)methyl)-1H-indole-3-carbonitrile as brown liquid (4.6 g, Yield: 66%).
TLC system: EtOAc/Hexane (40:90),
R_{f} value: ˜0.6;

¹H NMR (400 MHz, CDCl₃) δ: 7.67 (s, 1H), 7.20-7.18 (m, 1H), 5.45 (s, 2H), 3.48 (t, J=8.0 Hz, 2H), 0.90 (t, J=8.0 Hz, 2H), −0.04 (s, 9H).

### Synthesis of (Z)-5-bromo-4,6-difluoro-N′-hydroxy-1-((2-(trimethylsilyl)ethoxy)methyl)-1H-indole-3-carboximidamide

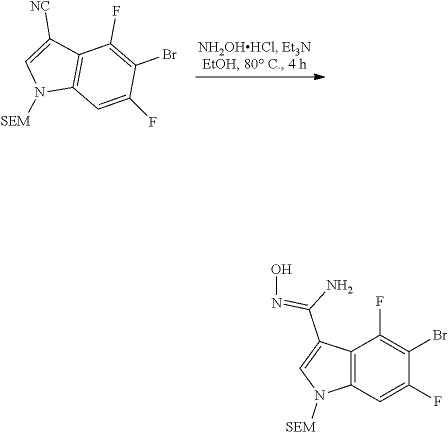 [SMILES: C[Si](C)(C)CCOCn1cc(C#N)c2c(F)c(Br)c(F)cc21 ; C[Si](C)(C)CCOCn1cc(/C(N)=N/O)c2c(F)c(Br)c(F)cc21]

To a stirred solution of 5-bromo-4,6-difluoro-1-((2-(trimethylsilyl)ethoxy)methyl)-1H-indole-3-carbonitrile (4.6 g, 11.89 mmol, 1.0 eq) in EtOH (46 mL) at room temperature, was added Et₃N (2.5 mL, 17.8 mmol, 1.5 eq) and NH₂OH·HCl (0.9 g, 17.8 mmol, 1.5 eq). The reaction mixture was stirred at 80° C. for 4 h. After completion of reaction by TLC, the reaction mixture was diluted with water (35 mL) and extracted with EtOAc (2×100 mL). The combined organic layer was dried over Na₂SO₄, filtered, and concentrated under reduced pressure to afford crude product. The crude product was triturated with n-pentane (30 mL), decanted and dried under vacuum to afford (Z)-5-bromo-4,6-difluoro-N′-hydroxy-1-((2-(trimethylsilyl)ethoxy)methyl)-1H-indole-3-carboximidamide as off white solid (4.28 g, Crude).
TLC system: EtOAc/Hexane (50:50),
R_{f} value: ˜0.2;

LCMS (m/z): 420.23 (M+H+2)⁺

### Synthesis of ethyl (Z)-((5-bromo-4,6-difluoro-1-((2-(trimethylsilyl)ethoxy)methyl)-1H-indol-3-yl)(hydroxyimino)methyl)carbamate

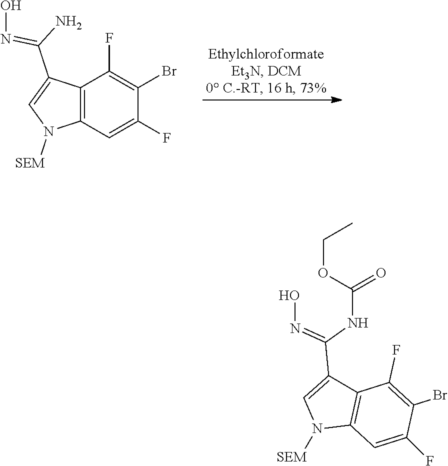 [SMILES: C[Si](C)(C)CCOCn1cc(/C(N)=N/O)c2c(F)c(Br)c(F)cc21 ; CCOC(=O)N/C(=N\O)c1cn(COCC[Si](C)(C)C)c2cc(F)c(Br)c(F)c12]

To a stirred solution of (Z)-5-bromo-4,6-difluoro-N′-hydroxy-1-((2-(trimethylsilyl)ethoxy)methyl)-1H-indole-3-carboximidamide (4.2 g, 10.0 mmol, 1.0 eq) in DCM (84 mL, 20 Vol) at 0° C., was added Et₃N (1 mL, 13.0 mmol, 1.3 eq) and 2-ethylchloroformate (1.14 mL, 12.0 mmol, 1.2 eq). The reaction mixture was stirred at room temperature for 16 h. After completion of reaction by TLC, the reaction mixture was quenched with NaHCO₃ solution (30 mL) and extracted with EtOAc (2×75 mL). The organic layer was dried over Na₂SO₄, filtered, and concentrated to afford crude product. The crude product was purified by silica gel (100-200 mesh) column chromatography [elution with 30% EtOAc in Hexane] to afford ethyl (Z)-((5-bromo-4,6-difluoro-1-((2-(trimethylsilyl)ethoxy)methyl)-1H-indol-3-yl)(hydroxyimino)methyl)carbamate as a brown gummy solid (3.6 g, Yield: 73%).
TLC system: EtOAc/Hexane (50:50),
R_{f} value: ˜0.5;

LCMS (m/z): 492.32 (M+H)⁺;

¹H NMR (400 MHz, CDCl₃) δ: 7.80 (s, 1H), 7.25-7.23 (m, 1H), 5.44 (s, 4H), 4.37 (q, J=7.2 Hz, 2H), 3.51 (t, J=8.0 Hz, 2H), 1.41 (t, J=7.2 Hz, 2H), 0.93 (t, J=8.0 Hz, 2H), 0.01 (s, 9H).

### Synthesis of 3-(5-bromo-4,6-difluoro-1-((2-(trimethylsilyl)ethoxy)methyl)-1H-indol-3-yl)-1,2,4-oxadiazol-5(4H)-one

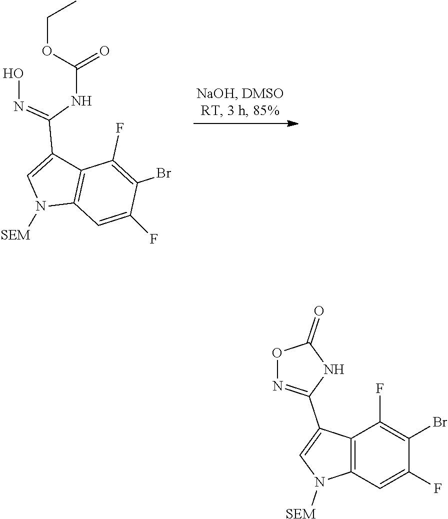 [SMILES: CCOC(=O)N/C(=N\O)c1cn(COCC[Si](C)(C)C)c2cc(F)c(Br)c(F)c12 ; C[Si](C)(C)CCOCn1cc(-c2noc(=O)[nH]2)c2c(F)c(Br)c(F)cc21]

To a stirred solution of ethyl (Z)-((5-bromo-4,6-difluoro-1-((2-(trimethylsilyl)ethoxy)methyl)-1H-indol-3-yl)(hydroxyimino)methyl)carbamate (3.6 g, 7.89 mmol, 1.0 eq) in DMSO (13 mL) at 0° C., was added NaOH (378 mg, 9.46 mmol, 1.2 eq) and stirred for 3 h at room temperature. After completion of reaction by TLC, the reaction mixture was diluted with water (30 mL), quenched with 2N HCl solution (5 mL) and extracted with EtOAc (2×100 mL). The combined organic layer was dried over Na₂SO₄, filtered, and concentrated under reduced pressure. The crude product was triturated with diethyl ether (20 mL), decanted and dried under vacuum to afford 3-(5-bromo-4,6-difluoro-1-((2-(trimethylsilyl)ethoxy)methyl)-1H-indol-3-yl)-1,2,4-oxadiazol-5(4H)-one as a brown solid (3 g, Yield: 85%).
TLC system: EtOAc/Hexane (50:50),
R_{f} value: ˜0.3;

LCMS (m/z): 446.2 (M+H)⁺;

¹H NMR (400 MHz, CDCl₃) δ: 10.21 (br, 1H), 7.88 (s, 1H), 7.27-7.25 (m, 1H), 5.51 (s, 2H), 3.54 (t, J=8.0 Hz, 2H), 0.95 (t, J=8.0 Hz, 2H), −0.01 (s, 9H).

### Synthesis of 3-(5-bromo-4,6-difluoro-1H-indol-3-yl)-1,2,4-oxadiazol-5(4H)-one

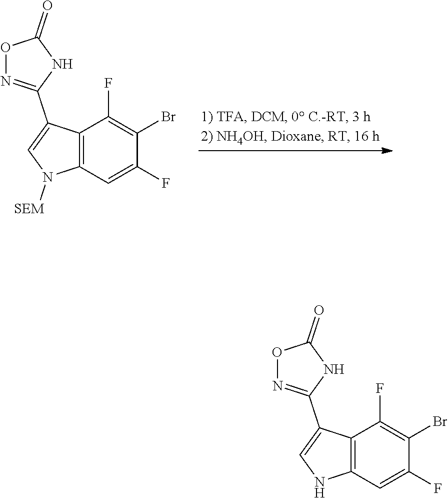 [SMILES: C[Si](C)(C)CCOCn1cc(-c2noc(=O)[nH]2)c2c(F)c(Br)c(F)cc21 ; O=c1[nH]c(-c2c[nH]c3cc(F)c(Br)c(F)c23)no1]

To a stirred solution of 3-(5-bromo-4,6-difluoro-1-((2-(trimethylsilyl)ethoxy)methyl)-1H-indol-3-yl)-1,2,4-oxadiazol-5(4H)-one (3 g, 6.74 mmol, 1.0 eq) in DCM (30 mL) at 0° C., was added TFA (15 mL, 5 Vol) and stirred at room temperature for 3h. Later, reaction mixture was filtered, washed with ether (10 mL) and collected solid was dissolved in aq. NH₃ (17 mL), 1,4 Dioxane (17 mL) and stirred at room temperature for 16 h. Reaction mass was concentrated under reduced pressure, diluted with water (20 mL), solid was filtered, washed with ether (10 mL) and dried under vacuum to afford 3-(5-bromo-4,6-difluoro-1H-indol-3-yl)-1,2,4-oxadiazol-5(4H)-one as off white solid (1 g, crude).
TLC system: EtOAc (100%),
R_{f} value: ˜0.3;

LCMS (m/z): 313.9 (M−H)⁺.

¹H NMR (400 MHz, DMSO-d₆) δ: 12.4 (s, 1H), 8.04 (s, 1H), 7.47-7.44 (m, 1H).

### Synthesis of 3-(4,6-difluoro-5-(4-(1-hydroxycyclobutyl)phenyl)-1H-indol-3-yl)-1,2,4-oxadiazol-5(4H)-one

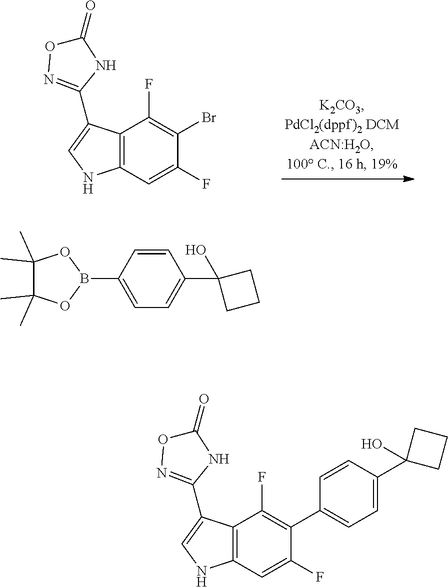 [SMILES: O=c1[nH]c(-c2c[nH]c3cc(F)c(Br)c(F)c23)no1 ; CC1(C)OB(c2ccc(C3(O)CCC3)cc2)OC1(C)C ; O=c1[nH]c(-c2c[nH]c3cc(F)c(-c4ccc(C5(O)CCC5)cc4)c(F)c23)no1]

In a sealed tube, to a degassed solution of 3-(5-bromo-4,6-difluoro-1H-indol-3-yl)-1,2,4-oxadiazol-5(4H)-one (150 mg, 0.47 mmol, 1.0 eq) and 1-(4-(4,4,5,5-tetramethyl-1,3,2-dioxaborolan-2-yl)phenyl)cyclobutan-1-ol (260 mg, 0.95 mmol, 2 eq) in ACN:H₂O (1:1, 3 mL) at room temperature, was added K₂CO₃ (131 mg, 0.95 mmol, 2.0 eq) followed by Pd(dppf)Cl₂·DCM (38 mg, 0.047 mmol, 0.1 eq) and stirred at 100° C. for 16 h. After completion of reaction by TLC, the reaction mixture was diluted with water (20 mL) and extracted with 10% MeOH/DCM (2×30 mL). The combined organic layer was dried over Na₂SO₄, filtered, and concentrated under reduced pressure to afford crude product. The crude product was purified by Prep-HPLC and lyophilized to afford 3-(4,6-difluoro-5-(4-(1-hydroxycyclobutyl)phenyl)-1H-indol-3-yl)-1,2,4-oxadiazol-5(4H)-one as a white solid (35 mg, Yield: 19%).
TLC system: EtOAc (100),
R_{f} value: ˜0.4;

LCMS (m/z): 382.1 (M−H)⁺;

¹H NMR (400 MHz, DMSO-d₆) δ: 12.55 (s, 1H), 12.3 (s, 1H), 8.02 (s, 1H), 7.60 (d, J=8.4 Hz, 2H), 7.43 (d, J=8.0 Hz, 2H), 7.35 (d, J=9.6 Hz, 1H), 5.56 (s, 1H), 2.46-2.41 (m, 2H), 2.34-2.27 (m, 2H), 1.99-1.95 (m, 1H), 1.75-1.72 (m, 1H).

### Prep Conditions:

SAMPLE NAME: C0189-34-P
COLUMN NAME: X-BRIDGE (250×19) mm 5 MICRON
Buffer MP-(A): 0.1% FA in WATER MP-(B): 100% ACN
Gradient (T/% B): 0/30, 15/60, 25/95
Flow Rate: 13 mL/min
Solubility: water, acetonitrile

### SYNTHETIC EXAMPLE 9

### Synthesis of Compound I-12

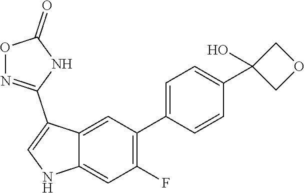 [SMILES: O=c1[nH]c(-c2c[nH]c3cc(F)c(-c4ccc(C5(O)COC5)cc4)cc23)no1]

### Synthesis of 3-(4-(4,4,5,5-tetramethyl-1,3,2-dioxaborolan-2-yl)phenyl)oxetan-3-ol

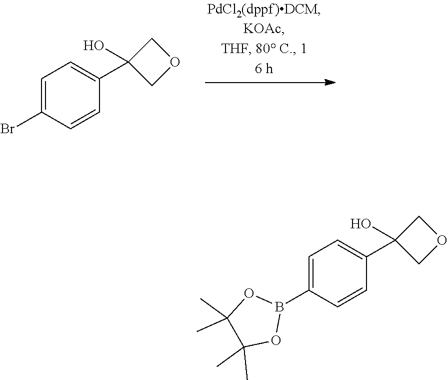 [SMILES: OC1(c2ccc(Br)cc2)COC1 ; CC1(C)OB(c2ccc(C3(O)COC3)cc2)OC1(C)C]

In a sealed tube, to a degassed solution of 3-(4-bromophenyl)oxetan-3-ol (1 g, 4.36 mmol, 1.0 eq) and B₂(pin)₂ (1.33 g, 5.23 mmol, 1.2 eq) in THF (20 mL) at room temperature, was added KOAc (1.28 g, 13.1 mmol, 3.0 eq), Pd(dppf)Cl₂ DCM (178 mg, 0.21 mmol, 0.05 eq) and stirred at 80° C. for 16 h. After completion of reaction by TLC, reaction mixture was filtered through a pad of diatomaceous earth (e.g., Celite®), washed with EtOAc (10 mL), collected filtrate was diluted with water (20 mL) and extracted with EtOAc (2×30 mL). The combined organic layer was dried over Na₂SO₄, filtered and concentrated under reduced pressure to afford 3-(4-(4,4,5,5-tetramethyl-1,3,2-dioxaborolan-2-yl)phenyl)oxetan-3-ol as off white solid (1 g, Yield: Crude).
TLC system: EtOAc/Hexane (50:50),
R_{f} value: ˜0.2;

¹H NMR (400 MHz, CDCl₃) δ: 7.87 (d, J=8.4 Hz, 2H), 7.61 (d, J=8.4 Hz, 2H), 4.91 (s, 4H), 1.35 (s, 12H).

### Synthesis of 3-(6-fluoro-5-(4-(3-hydroxyoxetan-3-yl)phenyl)-1H-indol-3-yl)-1,2,4-oxadiazol-5(4H)-one

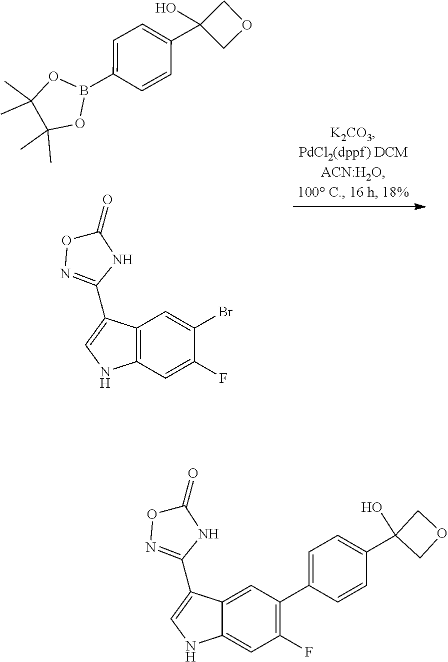 [SMILES: CC1(C)OB(c2ccc(C3(O)COC3)cc2)OC1(C)C ; O=c1[nH]c(-c2c[nH]c3cc(F)c(Br)cc23)no1 ; O=c1[nH]c(-c2c[nH]c3cc(F)c(-c4ccc(C5(O)COC5)cc4)cc23)no1]

In a sealed tube, to a degassed solution of 3-(5-bromo-6-fluoro-1H-indol-3-yl)-1,2,4-oxadiazol-5(4H)-one (250 mg, 0.84 mmol, 1.0 eq) and 3-(4-(4,4,5,5-tetramethyl-1,3,2-dioxaborolan-2-yl)phenyl)oxetan-3-ol (374 mg, 1.34 mmol, 1.6 eq) in ACN:H₂O (1:1, 5 mL) at room temperature, was added K₂CO₃ (348 mg, 2.52 mmol, 3.0 eq), Pd(dppf)Cl₂DCM (68 mg, 0.08 mmol, 0.1 eq) and stirred at 100° C. for 16 h. After completion of reaction by TLC, reaction mixture was diluted with water (20 mL) and extracted with 20% MeOH/DCM (3× 20 mL). The combined organic layer was dried over Na₂SO₄, filtered and concentrated under reduced pressure to afford crude product. The crude product was purified by silica column chromatography [elution with 10-15% MeOH/DCM] followed by Prep-HPLC and lyophilizing the fractions afforded 3-(6-fluoro-5-(4-(3-hydroxyoxetan-3-yl)phenyl)-1H-indol-3-yl)-1,2,4-oxadiazol-5(4H)-one as white solid (55 mg, Yield: 18%).
TLC system: EtOAc (100),
R_{f} value: ˜0.4;

LCMS (m/z): 366.1 (M−H)⁺;

¹H NMR (400 MHz, DMSO-d₆) δ: 12.66 (s, 1H), 12.02 (s, 1H), 8.02 (d, J=2.8 Hz, 1H), 7.94 (d, J=7.6 Hz, 1H), 7.72 (d, J=8.4 Hz, 2H), 7.58 (dd, J=1.6 Hz, 8.4 Hz, 2H), 7.48 (d, J=11.2 Hz, 1H), 6.41 (s, 1H), 4.81 (d, J=6.8 Hz, 2H), 4.74 (d, J=6.8 Hz, 2H).

### Prep Conditions

SUNFIRE C-18 (250×19 mm) 5μ
Buffer: MP-(A): 0.1% TFA IN WATER MP-(B): ACN
Gradient: (T/% B) 0.1/30, 15/60, 25/95.
Flow Rate: 13 mL/min
Solubility: water, acetonitrile, THF

### SYNTHETIC EXAMPLE 10

### Synthesis of Compound I-13

### Synthesis of 3-(4,6-difluoro-5-(4-(3-hydroxyoxetan-3-yl)phenyl)-1H-indol-3-yl)-1,2,4-oxadiazol-5(4H)-one

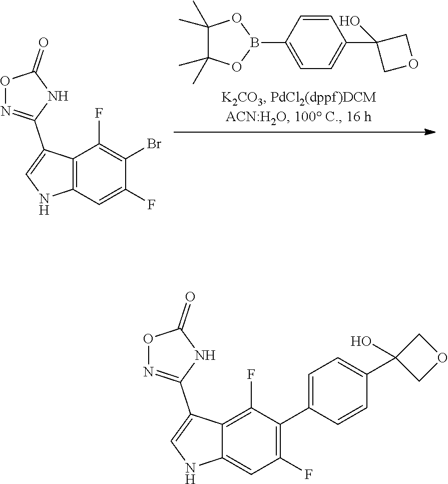 [SMILES: O=c1[nH]c(-c2c[nH]c3cc(F)c(Br)c(F)c23)no1 ; CC1(C)OB(c2ccc(C3(O)COC3)cc2)OC1(C)C ; O=c1[nH]c(-c2c[nH]c3cc(F)c(-c4ccc(C5(O)COC5)cc4)c(F)c23)no1]

To a stirred solution of 3-(5-bromo-4,6-difluoro-1H-indol-3-yl)-1,2,4-oxadiazol-5(4H)-one (150 mg, 0.47 mmol, 1.0 eq) in ACN:Water (20 vol, 1:1 ratio, 3 mL), was added 3-(4-(4,4,5,5-tetramethyl-1,3,2-dioxaborolan-2-yl)phenyl)oxetan-3-ol (265 mg, 0.95 mmol, 2 eq), K₂CO₃ (198 mg, 0.95 mmol, 2 eq). Reaction mixture was degassed with N₂ for 10 min then added Pd(dppf)Cl₂·DCM (39 mg, 0.047 mmol, 0.1 eq). The reaction mixture was stirred at 100° C. for 16 h. After completion of reaction by TLC, the reaction mixture was diluted with water (20 mL) and extracted with EtOAc (2×20 mL). The combined organic layer was dried over Na₂SO₄, filtered, and concentrated under reduced pressure to afford crude product. The crude product was purified reverse phase purification and further purified by Prep-HPLC to afford 3-(4,6-difluoro-5-(4-(3-hydroxyoxetan-3-yl)phenyl)-1H-indol-3-yl)-1,2,4-oxadiazol-5(4H)-one as Off-white solid (23 mg, Yield 12%),
TLC system: 10% MeOH in DCM,
R_{f} value: ˜0.5;

LCMS (m/z): 99.84%, 384.1 (M−H)⁻.

¹H NMR (400 MHz, DMSO-d₆) δ: 12.32 (s, 1H), 8.02 (s, 1H), 7.73 (d, J=8.4 Hz, 2H), 7.50 (d, J=8.0 Hz, 2H), 7.36 (d, J=9.6 Hz, 1H), 6.45 (s, 1H), 4.81 (d, J=6.8 Hz, 2H), 4.75 (d, J=6.8 Hz, 2H)

### Prep Conditions:

X-BRIDGE (250×19) mm 5 MICRON
Buffer MP-(A): 0.1% FA in WATER MP-(B): 100% ACN Gradient (T/% B) 0/20, 15/40, 25/95
Flow Rate 15 mL/min
Solubility: water, acetonitrile, THF

### SYNTHETIC EXAMPLE 11

### Synthesis of Compound I-14

### Synthesis of 3-(5-(2,6-dimethoxypyridin-3-yl)-6-fluoro-1H-indol-3-yl)-1,2,4-oxadiazol-5(4H)-one

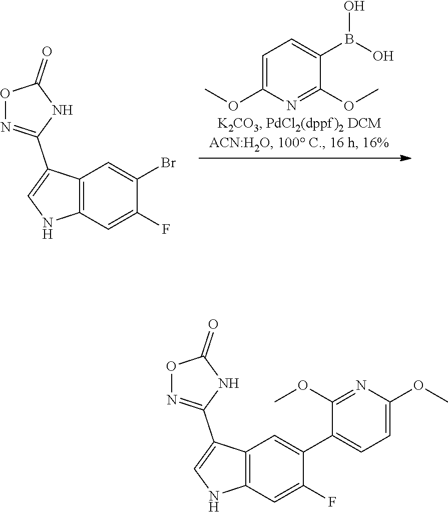 [SMILES: O=c1[nH]c(-c2c[nH]c3cc(F)c(Br)cc23)no1 ; COc1ccc(B(O)O)c(OC)n1 ; COc1ccc(-c2cc3c(-c4noc(=O)[nH]4)c[nH]c3cc2F)c(OC)n1]

In a sealed tube to a degassed solution of 3-(5-bromo-6-fluoro-1H-indol-3-yl)-1,2,4-oxadiazol-5(4H)-one (250 mg, 0.84 mmol, 1.0 eq) and (2,6-dimethoxypyridin-3-yl) boronic acid (308 mg, 1.68 mmol, 2 eq) in ACN:H₂O (1:1, 5 mL) at room temperature, was added K₂CO₃ (232 mg, 1.68 mmol, 2.0 eq) followed by Pd(dppf)Cl₂·DCM (68 mg, 0.084 mmol, 0.1 eq) and stirred at 100° C. for 16 h. After completion of reaction by TLC, the reaction mixture was diluted with water (20 mL) and extracted with 15% MeOH/DCM (3×20 mL). The combined organic layer was dried over Na₂SO₄, filtered and concentrated under reduced pressure to afford crude product. The crude product was purified by reverse phase column chromatography [elution with 35% ACN in 0.1% FA in H₂O] to 3-(5-(2,6-dimethoxypyridin-3-yl)-6-fluoro-1H-indol-3-yl)-1,2,4-oxadiazol-5(4H)-one as white solid (50 mg, Yield: 16%).
TLC system: EtOAc/Hexane (70:30),
R_{f} value: ˜0.25;

LCMS (m/z): 357.2 (M+H)⁺;

¹H NMR (400 MHz, DMSO-d₆) δ: 11.92 (s, 1H), 7.95 (d, J=2.8 Hz, 1H), 7.78 (d, J=7.6 Hz, 1H), 7.62 (d, J=8.0 Hz, 1H), 7.38 (d, J=10.4 Hz, 1H), 6.48 (d, J=8.0 Hz, 1H), 3.92 (s, 3H), 3.85 (s, 3H).

### SYNTHETIC EXAMPLE 12

### Synthesis of Compound I-15

### Synthesis of 3-(6-fluoro-5-(2-methoxypyridin-3-yl)-1H-indol-3-yl)-1,2,4-oxadiazol-5(4H)-one

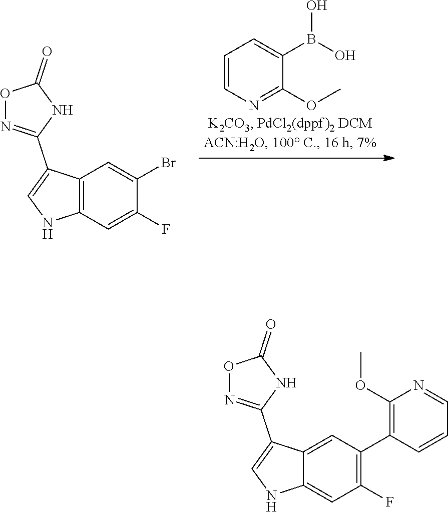 [SMILES: O=c1[nH]c(-c2c[nH]c3cc(F)c(Br)cc23)no1 ; COc1ncccc1B(O)O ; COc1ncccc1-c1cc2c(-c3noc(=O)[nH]3)c[nH]c2cc1F]

In a sealed tube to a degassed solution of 3-(5-bromo-6-fluoro-1H-indol-3-yl)-1,2,4-oxadiazol-5(4H)-one (250 mg, 0.84 mmol, 1.0 eq) and (2-methoxypyridin-3-yl) boronic acid (257 mg, 1.68 mmol, 2 eq) in ACN:H₂O (1:1, 5 mL) at room temperature, was added K₂CO₃ (232 mg, 1.68 mmol, 2.0 eq) followed by Pd(dppf)Cl₂·DCM (68 mg, 0.084 mmol, 0.1 eq) and stirred at 100° C. for 16 h. After completion of reaction by TLC, the reaction mixture was diluted with water (20 mL) and extracted with 15% MeOH/DCM (3×20 mL). The combined organic layer was dried over Na₂SO₄, filtered, and concentrated under reduced pressure to afford crude product. The crude product was purified by reverse phase column chromatography [elution with 35% ACN in 0.1% FA in H₂O] to afford 37 mg of desired product (¹H NMR aromatic impurities). This was re-purified by Prep-HPLC to afford 3-(6-fluoro-5-(2-methoxypyridin-3-yl)-1H-indol-3-yl)-1,2,4-oxadiazol-5(4H)-one as white solid (20 mg, Yield: 7%).
TLC system: EtOAc/Hexane (70:30),
R_{f} value: ˜0.3;

LCMS (m/z): 327.1 (M+H)⁺;

¹H NMR (400 MHz, DMSO-d₆) δ: 12.66 (br s, 1H), 12.01 (s, 1H), 8.33 (s, 1H), 8.00 (s, 1H), 7.92-7.87 (m, 2H), 7.49 (d, J=11.2 Hz, 1H), 6.94 (d, J=8.4 Hz, 1H), 3.91 (s, 3H).

### Prep Conditions:

Column: GEMINI C₁₈ (250× 21.2) mm 5mic
Buffer: MP-(A): 10 mM ABC IN WATER, MP-(B): ACN
Gradient (T/% B): 0.1/5, 5/5, 15/65, 25/95 Flow Rate: 14 mL/min Solubility: water, acetonitrile, THF

### SYNTHETIC EXAMPLE 13

### Synthesis of Compound I-16

### Synthesis of 3-(6-fluoro-5-(6-methoxypyridin-3-yl)-1H-indol-3-yl)-1,2,4-oxadiazol-5(4H)-one

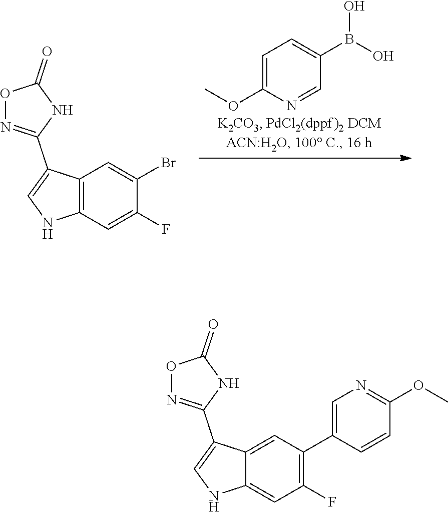 [SMILES: O=c1[nH]c(-c2c[nH]c3cc(F)c(Br)cc23)no1 ; COc1ccc(B(O)O)cn1 ; COc1ccc(-c2cc3c(-c4noc(=O)[nH]4)c[nH]c3cc2F)cn1]

In a sealed tube to a degassed solution of 3-(5-bromo-6-fluoro-1H-indol-3-yl)-1,2,4-oxadiazol-5(4H)-one (250 mg, 0.84 mmol, 1.0 eq) and (6-methoxypyridin-3-yl) boronic acid (395 mg, 1.68 mmol, 2 eq) in ACN:H₂O (1:1, 5 mL) at room temperature, was added K₂CO₃ (232 mg, 1.68 mmol, 2.0 eq) followed by Pd(dppf)Cl₂·DCM (68 mg, 0.084 mmol, 0.1 eq) and stirred at 100° C. for 16 h. After completion of reaction by TLC, reaction mass was concentrated under reduced pressure and purified by reverse phase column chromatography [elution with 40% ACN in 0.1% FA in H₂O] to afford 120 mg of desired product (LCMS: 57% purity). This was re-purified by Prep-HPLC to afford 3-(6-fluoro-5-(6-methoxypyridin-3-yl)-1H-indol-3-yl)-1,2,4-oxadiazol-5(4H)-one as off white solid (40 mg, Yield: 14%).
TLC system: EtOAc (100%),
R_{f} value: ˜0.5;

LCMS (m/z): 327.1 (M+H)⁺;

¹H NMR (400 MHz, DMSO-d₆) δ: 12.65 (br s, 1H), 12.04 (s, 1H), 8.24 (dd, J=2 Hz, 5.2 Hz, 1H), 8.01 (d, J=2.8 Hz, 1H), 7.80 (d, J=7.2 Hz, 1H), 7.71 (dd, J=2 Hz, 7.2 Hz, 1H), 7.44 (d, J=10.4 Hz, 1H), 7.13-7.10 (m, 1H), 3.83 (s, 3H).

### Prep Conditions:

Column: sunfire C₁₈ (250× 19) mm 5mic
Buffer: MP-(A): 0.1% TFA in water MP-(B): ACN
Gradient (T/% B): 0.1/40, 13/55, 14/95, 18/95, 18.20/40, 22/40
Flow Rate: 12 mL/min
Solubility: ACN, Water, THF

### SYNTHETIC EXAMPLE 14

### Synthesis of Compound I-18

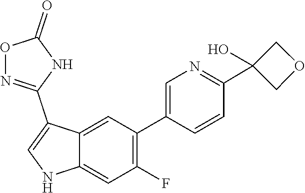 [SMILES: O=c1[nH]c(-c2c[nH]c3cc(F)c(-c4ccc(C5(O)COC5)nc4)cc23)no1]

### Synthesis of 3-(5-(tributylstannyl) pyridin-2-yl)oxetan-3-o

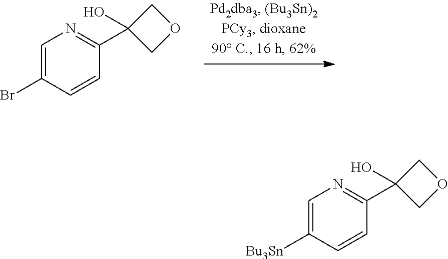

In a sealed tube to a degassed solution of 3-(5-bromopyridin-2-yl)oxetan-3-ol (500 mg, 2.17 mmol, 1.0 eq) and (Bu₃Sn)₂ (1.89 g, 3.26 mmol, 1.5 eq) in 1,4-dioxane (10 mL) at room temperature, was added PCy₃ (60 mg, 0.21 mmol, 0.1 eq) followed by Pd₂(dba)₃ (99 mg, 0.11 mmol, 0.05 eq) and stirred at 90° C. for 16 h. After completion of reaction by TLC, the reaction mixture was diluted with water (40 mL) and extracted with EtOAc (2×50 mL). The combined organic layer was dried over Na₂SO₄, filtered, and concentrated under reduced pressure. The crude product was purified by silica gel (100-200 mesh) column chromatography [elution with 25-30% EtOAc in Hexane] to afford 3-(5-(tributylstannyl) pyridin-2-yl)oxetan-3-ol as a brown liquid (600 mg, Yield: 62%).
TLC system: EtOAc/Hexane (30:70),
R_{f} value: ˜0.5;

¹H NMR (400 MHz, CDCl₃) δ: 8.49 (s, 1H), 7.96-7.92 (m, 2H), 5.09 (d, J=7.2 Hz, 2H), 4.73 (d, J=7.2 Hz, 2H), 1.57-1.51 (m, 6H), 1.39-1.30 (m, 6H), 1.14-1.10 (m, 6H), 0.98-0.85 (m, 9H).

### Synthesis of 3-(6-fluoro-5-(6-(3-hydroxyoxetan-3-yl) pyridin-3-yl)-1H-indol-3-yl)-1,2,4-oxadiazol-5(4H)-one

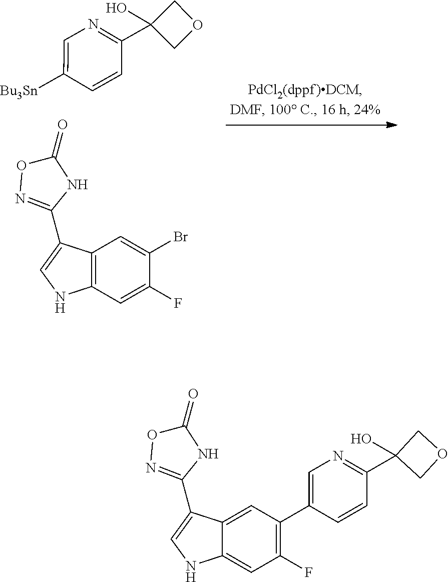

In a sealed tube, to a degassed solution of 3-(5-bromo-6-fluoro-1H-indol-3-yl)-1,2,4-oxadiazol-5(4H)-one (200 mg, 0.67 mmol, 1.0 eq) and 3-(5-(tributylstannyl) pyridin-2-yl)oxetan-3-ol (445 mg, 1.01 mmol, 1.5 eq)) in DMF (4 mL) at room temperature, was added Pd(dppf)Cl₂·DCM (28 mg, 0.033 mmol, 0.05 eq) and stirred at 100° C. for 16 h. After completion of reaction by TLC, the reaction mixture was diluted with water (15 mL) and extracted with 10% MeOH in DCM (2×30 mL). The combined organic layer was dried over Na₂SO₄, filtered and concentrated under reduced pressure. The crude product was purified by Prep-HPLC to afford 3-(6-fluoro-5-(6-(3-hydroxyoxetan-3-yl) pyridin-3-yl)-1H-indol-3-yl)-1,2,4-oxadiazol-5(4H)-one as off white solid (60 mg, Yield: 24%).
TLC system: EtOAc (100%),
R_{f} value: ˜0.3;

LCMS (m/z): 369.3 (M+H)⁺;

¹H NMR (400 MHz, DMSO-d₆) δ: 12.69 (br s, 1H), 12.09 (s, 1H), 8.81 (s, 1H), 8.05-7.97 (m, 3H), 7.72 (d, J=8.4 Hz, 1H), 7.55 (d, J=11.2 Hz, 1H), 6.63 (s, 1H), 4.98 (d, J=6.0 Hz, 2H), 4.70 (d, J=6.4 Hz, 2H).

### SYNTHETIC EXAMPLE 15

### Synthesis of Compound I-21

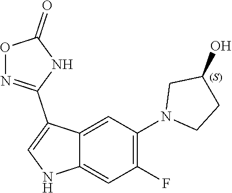

### Synthesis of(S)-5-(3-((tert-butyldimethylsilyl)oxy)pyrrolidin-1-yl)-6-fluoro-1-((2-(trimethylsilyl)ethoxy)methyl)-1H-indole-3-carbonitrile

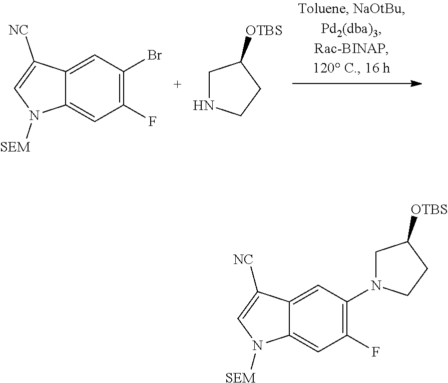 [SMILES: C[Si](C)(C)CCOCn1cc(C#N)c2cc(Br)c(F)cc21 ; CC(C)(C)[Si](C)(C)O[C@H]1CCNC1 ; CC(C)(C)[Si](C)(C)O[C@H]1CCN(c2cc3c(C#N)cn(COCC[Si](C)(C)C)c3cc2F)C1]

To a stirred solution of 5-bromo-6-fluoro-1-((2-(trimethylsilyl)ethoxy)methyl)-1H-indole-3-carbonitrile (2.0 g, 5.42 mmol, 1.0 eq) in Toluene (40 mL) at room temperature, was added (S)-3-((tert-butyldimethylsilyl)oxy)pyrrolidine (1.2 g, 5.96 mmol, 1.1 eq) and NaOtBu (1.04 g, 10.8 mmol, 2 eq) and Rac-BINAP (203 mg, 0.3 mmol, 0.06 eq), Reaction mixture was degassed with N₂ for 10 min then added Pd₂(dba)₃ (149 mg, 0.1 mmol, 0.03 eq). The reaction mixture was stirred at 120° C. for 16 h. After completion of reaction by TLC, the reaction mixture was diluted with water (35 mL) and extracted with EtOAc (2×50 mL). The combined organic layer was dried over Na₂SO₄, filtered and concentrated under reduced pressure to afford crude product. The crude product was purified by silica gel (100-200 mesh) column chromatography [elution with 30% EtOAc in Hexane] to afford (S)-5-(3-((tert-butyldimethylsilyl)oxy)pyrrolidin-1-yl)-6-fluoro-1-((2-(trimethylsilyl)ethoxy)methyl)-1H-indole-3-carbonitrile as a pale yellow liquid (1.0 g, Yield: 37%).
TLC system: EtOAc/Hexane (50:50),
R_{f} value: ˜0.8;

LCMS (m/z): 490.4 (M+H)⁺.

¹H NMR (400 MHz, CDCl₃) δ: 7.54 (s, 1H), 7.19 (d, J=13.2 Hz, 1H), 6.94 (d, J=8.0 Hz, 1H), 5.38 (s, 2H), 4.54-4.52 (m, 1H), 3.70-3.65 (m, 1H), 3.51-3.40 (m, 4H), 3.23-3.19 (m, 1H), 2.15-2.10 (m, 1H), 1.97-1.94 (m, 1H), 0.91 (s, 9H), 0.90-0.87 (m, 2H), 0.09 (s, 6H), −0.04 (s, 9H).

### Synthesis of(S)-5-(3-((tert-butyldimethylsilyl)oxy)pyrrolidin-1-yl)-6-fluoro-N-hydroxy-1-((2-(trimethylsilyl)ethoxy)methyl)-1H-indole-3-carboximidamide

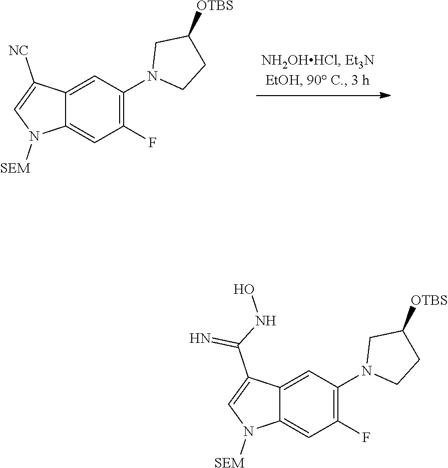 [SMILES: CC(C)(C)[Si](C)(C)O[C@H]1CCN(c2cc3c(C#N)cn(COCC[Si](C)(C)C)c3cc2F)C1 ; CC(C)(C)[Si](C)(C)O[C@H]1CCN(c2cc3c(C(=N)NO)cn(COCC[Si](C)(C)C)c3cc2F)C1]

To a stirred solution of(S)-5-(3-((tert-butyldimethylsilyl)oxy)pyrrolidin-1-yl)-6-fluoro-1-((2-(trimethylsilyl)ethoxy)methyl)-1H-indole-3-carbonitrile (1 g, 2.04 mmol, 1.0 eq) in EtOH (10 mL) at room temperature, was added Et₃N (0.7 mL, 4.09 mmol, 2 eq) and NH₂OH·HCl (284 mg, 4.09 mmol, 2 eq). The reaction mixture was stirred at 90° C. for 3 h. After completion of reaction by TLC, the reaction mixture was diluted with water (35 mL) and extracted with EtOAc (2×100 mL). The combined organic layer was dried over Na₂SO₄, filtered and concentrated under reduced pressure to afford crude product(S)-5-(3-((tert-butyldimethylsilyl)oxy)pyrrolidin-1-yl)-6-fluoro-N-hydroxy-1-((2-(trimethylsilyl)ethoxy)methyl)-1H-indole-3-carboximidamide as a color less liquid (950 mg, Crude).
TLC system: EtOAc/Hexane (30:70),
R_{f} value: ˜0.2;

LCMS (m/z): 523.4 (M+H)⁺.

¹H NMR (400 MHz, CDCl₃) δ: 7.36 (s, 1H), 7.31 (d, J=8.8 Hz, 1H), 7.14 (d, J=13.2 Hz, 1H), 5.35 (s, 2H), 4.54-4.52 (m, 1H), 3.68-3.65 (m, 1H), 3.50-3.38 (m, 4H), 3.19-3.16 (m, 1H), 2.16-1.89 (m, 2H), 0.91-0.86 (s, 11H), 0.09 (s, 3H), 0.08 (s, 3H), −0.05 (s, 9H).

### Synthesis of ethyl(S)-5-(3-((tert-butyldimethylsilyl)oxy)pyrrolidin-1-yl)-N-((ethoxycarbonyl)oxy)-6-fluoro-1-((2-(trimethylsilyl)ethoxy)methyl)-1H-indole-3-carboximidamide

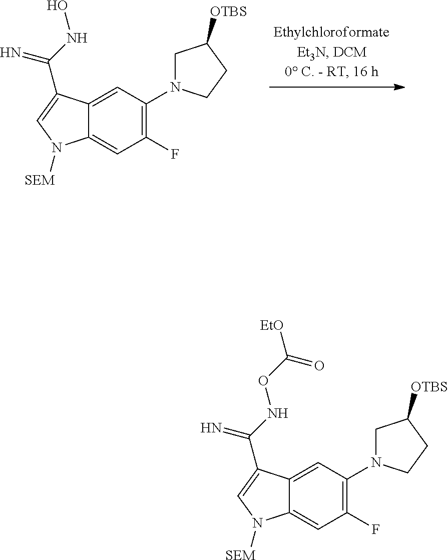 [SMILES: CC(C)(C)[Si](C)(C)O[C@H]1CCN(c2cc3c(C(=N)NO)cn(COCC[Si](C)(C)C)c3cc2F)C1 ; CCOC(=O)ONC(=N)c1cn(COCC[Si](C)(C)C)c2cc(F)c(N3CC[C@H](O[Si](C)(C)C(C)(C)C)C3)cc12]

To a stirred solution of(S)-5-(3-((tert-butyldimethylsilyl)oxy)pyrrolidin-1-yl)-6-fluoro-N-hydroxy-1-((2-(trimethylsilyl)ethoxy)methyl)-1H-indole-3-carboximidamide (950 mg, 1.82 mmol, 1.0 eq) in DCM (9.5 mL, 10 Vol) at 0° C., was added Et₃N (0.3 mL, 2.36 mmol, 1.3 eq) and 2-Ethylchloroformate (0.2 mL, 2.18 mmol, 1.2 eq). The reaction mixture was stirred at room temperature for 16 h. After completion of reaction by TLC, the reaction mixture was quenched with NaHCO₃ solution (10 mL) and extracted with EtOAc (2×75 mL). The organic layer was dried over Na₂SO₄, filtered, and concentrated to afford crude product. The crude product was purified by RP column afforded ethyl(S)-5-(3-((tert-butyldimethylsilyl)oxy)pyrrolidin-1-yl)-N-((ethoxycarbonyl)oxy)-6-fluoro-1-((2-(trimethylsilyl)ethoxy)methyl)-1H-indole-3-carboximidamide as a brown gummy solid (500 mg, Yield: 46%).
TLC system: EtOAc/Hexane (50:50),
R_{f} value: ˜0.8;

LCMS (m/z): 595.4 (M+H)⁺.

### Synthesis of(S)-3-(5-(3-((tert-butyldimethylsilyl)oxy)pyrrolidin-1-yl)-6-fluoro-1-((2-(trimethylsilyl)ethoxy)methyl)-1H-indol-3-yl)-1,2,4-oxadiazol-5(4H)-one

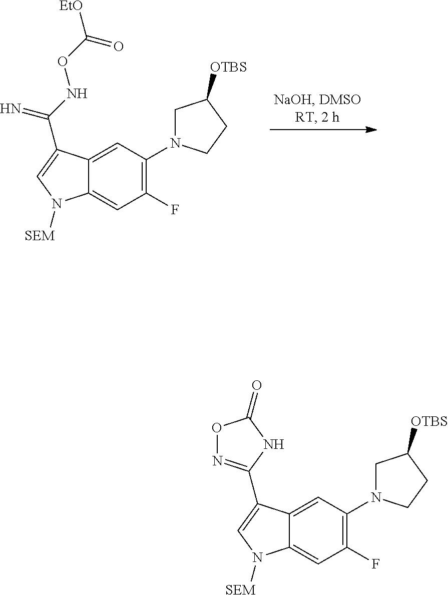 [SMILES: CCOC(=O)ONC(=N)c1cn(COCC[Si](C)(C)C)c2cc(F)c(N3CC[C@H](O[Si](C)(C)C(C)(C)C)C3)cc12 ; CC(C)(C)[Si](C)(C)O[C@H]1CCN(c2cc3c(-c4noc(=O)[nH]4)cn(COCC[Si](C)(C)C)c3cc2F)C1]

To a stirred solution of ethyl(S)-5-(3-((tert-butyldimethylsilyl)oxy)pyrrolidin-1-yl)-N-((ethoxycarbonyl)oxy)-6-fluoro-1-((2-(trimethylsilyl)ethoxy)methyl)-1H-indole-3-carboximidamide (500 mg, 0.84 mmol, 1.0 eq) in DMSO (1.75 mL) at 0° C., was added NaOH (40 mg, 1.01 mmol, 1.2 eq) and stirred for 2 h at room temperature. After completion of reaction by TLC, the reaction mixture was diluted with water (20 mL), quenched with 2N HCl solution (1 mL) resulting solid was filtered & dried to afford (S)-3-(5-(3-((tert-butyldimethylsilyl)oxy)pyrrolidin-1-yl)-6-fluoro-1-((2-(trimethylsilyl)ethoxy)methyl)-1H-indol-3-yl)-1,2,4-oxadiazol-5(4H)-one as a brown solid (400 mg, Yield: 36%).
TLC system: EtOAc/Hexane (50:50),
R_{f} value: ˜0.2;

LCMS (m/z): 549.50 (M+H)⁺.

### Synthesis of(S)-3-(5-(3-((tert-butyldimethylsilyl)oxy)pyrrolidin-1-yl)-6-fluoro-1-(hydroxymethyl)-1H-indol-3-yl)-1,2,4-oxadiazol-5(4H)-one

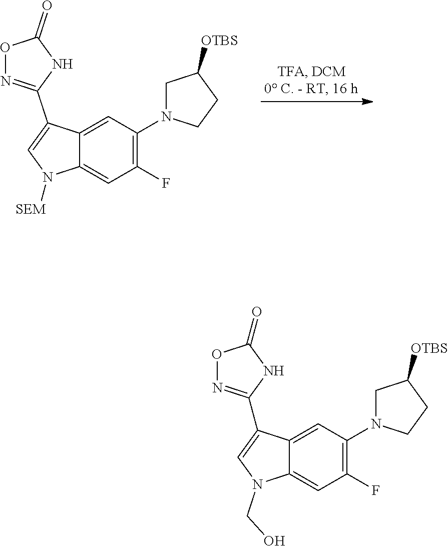 [SMILES: CC(C)(C)[Si](C)(C)O[C@H]1CCN(c2cc3c(-c4noc(=O)[nH]4)cn(COCC[Si](C)(C)C)c3cc2F)C1 ; CC(C)(C)[Si](C)(C)O[C@H]1CCN(c2cc3c(-c4noc(=O)[nH]4)cn(CO)c3cc2F)C1]

To a stirred solution of(S)-3-(5-(3-((tert-butyldimethylsilyl)oxy)pyrrolidin-1-yl)-6-fluoro-1-((2-(trimethylsilyl)ethoxy)methyl)-1H-indol-3-yl)-1,2,4-oxadiazol-5(4H)-one (200 mg, 0.36 mmol, 1.0 eq) in DCM (2 M, 10 Vol) at 0° C., was added TFA (2 mL, 10 Vol) and stirred at room temperature for 16 h. After completion of reaction by TLC, the reaction mixture concentrated, triturated with pentane (10 mL) to afford (S)-3-(5-(3-((tert-butyldimethylsilyl)oxy)pyrrolidin-1-yl)-6-fluoro-1-(hydroxymethyl)-1H-indol-3-yl)-1,2,4-oxadiazol-5(4H)-one as brown solid (350 mg, crude).
TLC system: EtOAc (100%),
R_{f} value: ˜0.2;

LCMS (m/z): 335.23 (M+H)⁺

### Synthesis of(S)-3-(6-fluoro-5-(3-hydroxypyrrolidin-1-yl)-1H-indol-3-yl)-1,2,4-oxadiazol-5(4H)-one

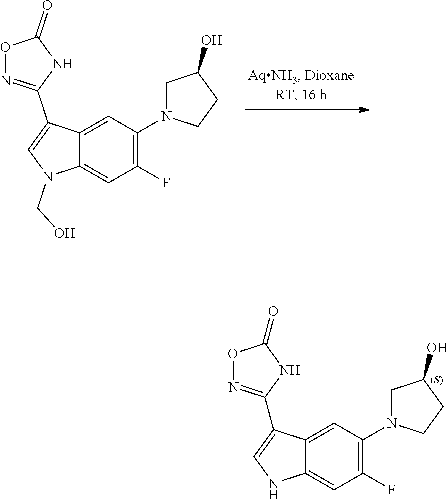

To a stirred solution of(S)-3-(5-(3-((tert-butyldimethylsilyl)oxy)pyrrolidin-1-yl)-6-fluoro-1-(hydroxymethyl)-1H-indol-3-yl)-1,2,4-oxadiazol-5(4H)-one (350 mg, 1.04 mmol, 1.0 eq) in Dioxane (3.5 mL) added aq. NH₃ (3.5 mL) and stirred at room temperature for 16 h. Reaction mass was concentrated under reduced pressure, diluted with water (20 mL), solid filtered, washed with water & ether (10 mL) and lyophilized to afford (S)-3-(6-fluoro-5-(3-hydroxypyrrolidin-1-yl)-1H-indol-3-yl)-1,2,4-oxadiazol-5(4H)-one as pale brown solid (82 mg, Yield: 26%).
TLC system: EtOAc (100%),
R_{f} value: ˜0.2;

LCMS (m/z): 305.1 [M+H]⁺.

¹H NMR (400 MHz, DMSO-d₆) δ: 12.55 (br, s, 1H), 11.66 (s, 1H), 7.84 (d, J=2.8 Hz, 1H), 7.28 (d, J=13.6 Hz, 1H), 7.17 (d, J=8.8 Hz, 1H), 4.91 (d, J=4.0 Hz, 1H), 4.37 (brs, 1H), 3.54-3.50 (m, 1H), 3.43-3.34 (m, 1H), 3.25-3.21 (m, 1H), 3.09-3.06 (m, 1H), 2.07-2.02 (m, 1H), 1.87-1.81 (m, 1H).

### SYNTHETIC EXAMPLE 16

### Synthesis of Compound I-23

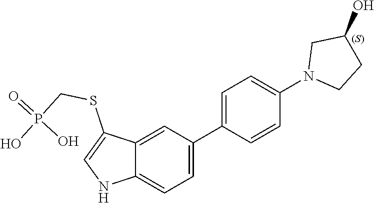

### Synthesis of 1,2-bis(5-bromo-1H-indol-3-yl)disulfane

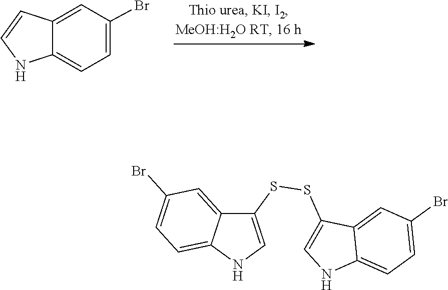 [SMILES: Brc1ccc2[nH]ccc2c1 ; Brc1ccc2[nH]cc(SSc3c[nH]c4ccc(Br)cc34)c2c1]

To a stirred solution of 5-bromo-1H-indole (5 g, 25.5 mmol, 1.0 eq) in MeOH (80 mL) Water (20 mL) at room temperature, was added Thio urea (1.93 g, 25.5 mmol, 1.0 eq), I₂ (6.32 g, 25.5 mmol, 1.0 eq) and KI (4.15 g, 25.5 mmol, 1.0 eq). The reaction mixture was stirred at room temperature for 16 h. After completion of reaction by TLC, the reaction mixture was treated with 2N aq. NaOH solution (10 mL) & THF (10 mL) (clear solution) and stirred at 100° C. for 30 min. RM was concentrated under reduced pressure. The reaction mixture was diluted with H₂O (50 mL), resulting precipitate was filtered and dried under vacuum. The crude product was triturated with diethyl ether (20 mL), filtered and dried under reduced pressure to afford 1,2-bis(5-bromo-1H-indol-3-yl)disulfane as an off white solid (2.2 g, Yield 38%).
TLC system: EtOAc/Hexane (50:50) or MeOH in DCM (10:90),
R_{f} value: ˜0.3;

LCMS (m/z): 80%, 450.87 (M−H)⁻.

### Synthesis of diethyl (((5-bromo-1H-indol-3-yl)thio)methyl)phosphonate

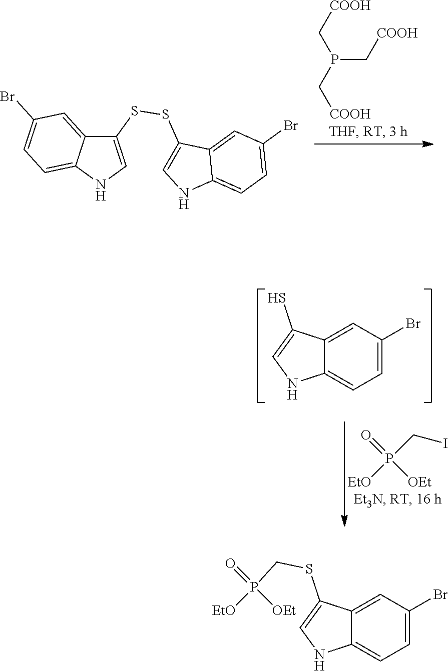

To a stirred solution of 1,2-bis(5-bromo-1H-indol-3-yl)disulfane (1.5 g, 3.31 mmol, 1.0 eq) in THF (15 mL) at RT, was added 2,2′,2″-phosphanetriyltriacetic acid (1.89 g, 6.62 mmol, 2.0 eq) and stirred for 16 h. After completion of reaction by TLC, & LC-MS. LC-MS indicated 75% m/z. Later, added diethyl (iodomethyl)phosphonate (2 g, 6.62 mmol, 2.0 eq) and TEA (2.3 mL, 16.5 mmol, 5 eq). The reaction mixture was stirred at room temperature for 16 h. After completion of reaction by TLC, quenched with water (20 mL) and extracted with EtOAc (2×20 mL). The combined organic layer was dried over Na₂SO₄, filtered and concentrated under reduced pressure. The crude product was purified by silica gel (100-200 mesh) column chromatography [elution with 0.5-1% MeOH in DCM] to afford diethyl (((5-bromo-1H-indol-3-yl)thio)methyl)phosphonate as a pale brown oil (0.5 g, 67% pure).
TLC system: EtOAc/Hexane (50:50),
R_{f} value: ˜0.3;

LCMS (m/z): 378.03 (M−H)⁺; 67% purity.

### Synthesis of diethyl(S)-(((5-(4-(3-hydroxypyrrolidin-1-yl)phenyl)-1H-indol-3-yl)thio)methyl)phosphonate

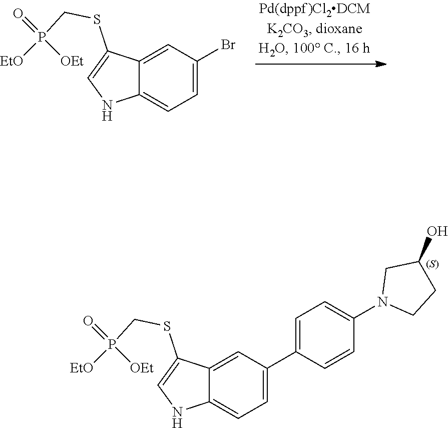

To a stirred solution of diethyl (((5-bromo-1H-indol-3-yl)thio)methyl)phosphonate (500 mg, 1.32 mmol, 1.0 eq) in dioxane (8 mL, 20 Vol), water (2 mL,), was added (S)-1-(4-(4,4,5,5-tetramethyl-1,3,2-dioxaborolan-2-yl)phenyl)pyrrolidin-3-ol (495 mg, 1.72 mmol, 1.3 eq), K₂CO₃ (538 mg, 3.96 mmol, 3 eq). Reaction mixture was degassed with N₂ for 10 min then added Pd(dppf)Cl₂·DCM (107 mg, 0.13 mmol, 0.1 eq). The reaction mixture was stirred at 100° C. for 16 h. After completion of reaction by TLC, the reaction mixture was diluted with water (20 mL) and extracted with EtOAc (2×20 mL). The combined organic layer was dried over Na₂SO₄, filtered, and concentrated under reduced pressure. The crude product was purified by silica gel (100-200 mesh) column chromatography [elution with 2-4% MeOH in DCM] to afford diethyl(S)-(((5-(4-(3-hydroxypyrrolidin-1-yl)phenyl)-1H-indol-3-yl)thio)methyl)phosphonate as a brown liquid (400 mg, semi-pure).
TLC system: EtOAc,
R_{f} value: ˜0.3;

LCMS (m/z): 461.37 (M+H)⁺; 66% purity.

### Synthesis of(S)-(((5-(4-(3-hydroxypyrrolidin-1-yl)phenyl)-1H-indol-3-yl)thio)methyl)phosphonic acid

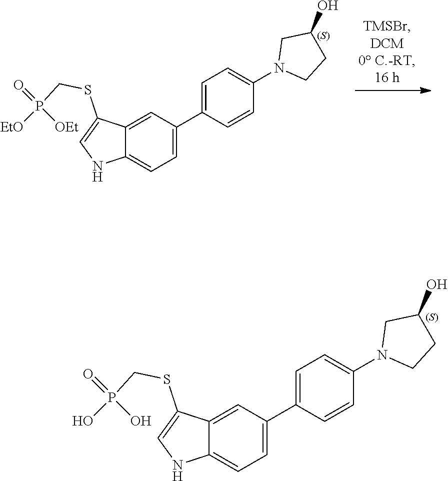

To a stirred solution of diethyl(S)-(((5-(4-(3-hydroxypyrrolidin-1-yl)phenyl)-1H-indol-3-yl)thio)methyl)phosphonate (200 mg, 0.45 mmol, 1.0 eq) in DCM (10 mL), at 0° C., was added TMSBr (0.5 mL) and stirred at room temperature for 16 h. After completion of reaction by TLC, the reaction mixture was concentrated, diluted with water (2 mL), basified with 2M NaOH solution, washed with EtOAc (2×10 mL). Aqueous layer was acidified with 2N aq. HCl (pH˜2) and evaporated under reduced pressure. The crude product was purified by prep-HPLC to afford (S)-(((5-(4-(3-hydroxypyrrolidin-1-yl)phenyl)-1H-indol-3-yl)thio)methyl)phosphonic acid as a white solid (29.5 mg, Yield 15%).
TLC system: MeOH/DCM (10:90),
R_{f} value: ^{˜}0;

LCMS (m/z): 403.16 (M−H)⁺. ¹H NMR (400 MHz, DMSO-d₆) δ: 11.31 (s, 1H), 7.80 (s, 1H), 7.54-7.51 (m, 3H), 7.43-7.36 (m, 2H), 6.60 (d, J=8.8 Hz, 2H), 4.99 (br, s, 1H), 4.43-4.40 (m, 1H), 3.47-3.28 (m, 3H), 3.13-3.10 (m, 1H), 2.86 (d, J=13.6 Hz, 2H), 2.09-2.04 (m, 1H). 1.93-1.91 (m, 1H).

### Prep-HPLC Method:

### Prep Conditions:

Column YMC C-18 (250×19) mm 5mic
Buffer MP-(A): 0.1% FA IN WATER MP-(B): CAN Gradient (T/% B) 0/25, 12/40, 12.5/95, 17/95, 18.1/25, 22/95
Flow Rate 12 mL/min
Solubility: acetonitrile, water

### SYNTHETIC EXAMPLE 17

### Synthesis of Compound I-24

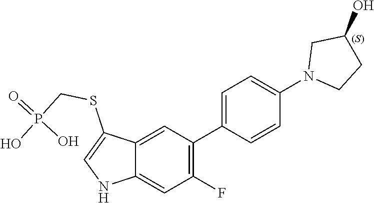

### Synthesis of 1,2-bis(5-bromo-6-fluoro-1H-indol-3-yl)disulfane

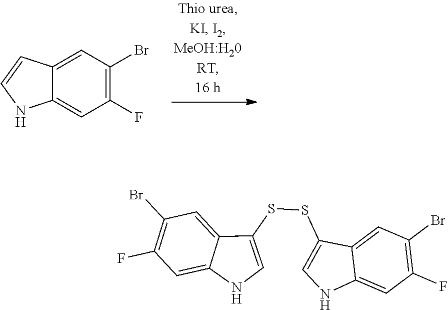 [SMILES: Fc1cc2[nH]ccc2cc1Br ; Fc1cc2[nH]cc(SSc3c[nH]c4cc(F)c(Br)cc34)c2cc1Br]

To a stirred solution of 5-bromo-6-fluoro-1H-indole (5 g, 23.5 mmol, 1.0 eq) in MeOH (80 mL), water (20 mL) at room temperature, was added Thio urea (1.75 g, 23.5 mmol, 1.0 eq), 12 (5.8 g, 23.5 mmol, 1.0 eq) and KI (3.8 g, 23.5 mmol, 1.0 eq). The reaction mixture was stirred at room temperature for 16 h. After completion of reaction by TLC, the reaction mixture was treated with 2N aq. NaOH solution (20 mL) & THF (20 mL) (clear solution) and stirred at 80° C. for 30 min. RM was concentrated under reduced pressure, diluted with H₂O (50 mL), resulting precipitate was filtered, triturated with diethyl ether (20 mL) and dried under vacuum to afford 1,2-bis(5-bromo-6-fluoro-1H-indol-3-yl)disulfane as off white solid (2.2 g, Yield 40%).
TLC system: MeOH in DCM (10:90),
R_{f} value: ˜0.3;

LCMS (m/z): 486.90 (M−H)⁺.

¹H NMR (400 MHz, DMSO-d₆) δ: 11.86 (brs, 1H), 7.54 (s, 1H), 7.43 (d, J=9.6 Hz, 1H), 7.16 (d, J=6.8 Hz, 1H).

### Synthesis of diethyl (((5-bromo-6-fluoro-1H-indol-3-yl)thio)methyl)phosphonate

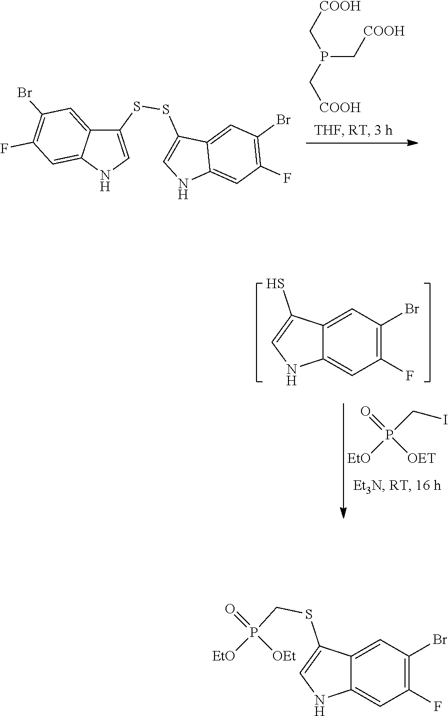

To a stirred solution of 1,2-bis(5-bromo-6-fluoro-1H-indol-3-yl)disulfane (1 g, 2.04 mmol, 1.0 eq) in THF (10 mL) was added 2,2′,2″-phosphanetriyltriacetic acid (1.17 g, 4.09 mmol, 2.0 eq) at room temperature and stirred for 16 h. Later, added diethyl (iodomethyl)phosphonate (1.13 mL, 4.09 mmol, 2.0 eq) and TEA (1.1 mL, 10.2 mmol, 5 eq) and stirred at room temperature for 16 h. After completion of reaction by TLC, quenched with water (20 mL) and extracted with EtOAc (2×20 mL). The combined organic layer was dried over Na₂SO₄, filtered, and concentrated under reduced pressure.

The crude product was purified by silica gel (100-200 mesh) column chromatography [elution with 0.5-1% MeOH in DCM] to afford diethyl (((5-bromo-6-fluoro-1H-indol-3-yl)thio)methyl)phosphonate as pale brown oil (0.5 g, Yield 61%).
TLC system: MeOH in DCM (10:90),
R_{f} value: ˜0.35;

LCMS (m/z): 396.16 (M+H)⁺;

¹H NMR (400 MHz, CDCl₃) δ: 9.92 (s, 1H), 7.87 (d, J=6.8 Hz, 1H), 7.36 (d, J=2.8 Hz, 1H), 7.07 (d, J=9.2 Hz, 1H), 4.20-4.11 (m, 4H), 2.95 (d, J=12.4 Hz, 2H), 1.34 (t, J=6.8 Hz, 6H).

### Synthesis of(S)-1-(4-(4,4,5,5-tetramethyl-1,3,2-dioxaborolan-2-yl)phenyl)pyrrolidin-3-ol

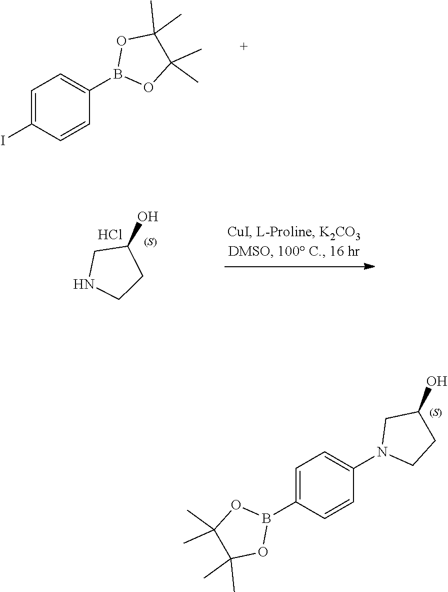

To a stirred solution of 2-(4-iodophenyl)-4,4,5,5-tetramethyl-1,3,2-dioxaborolane (2 g, 6.06 mmol, 1.0 eq) in DMSO (20 mL) at room temperature, was added (S)-pyrrolidin-3-ol HCl (2 g, 12.1 mmol, 2 eq), K₂CO₃ (1.6 g, 12.1 mmol, 2 eq). CuI (228 mg, 1.21 mmol, 0.2 eq) and L-proline (276 mg, 2.42 mmol, 0.4 eq). The reaction mixture was stirred at 100° C. for 16 h. After completion of reaction by TLC, the reaction mixture was diluted with ice water (50 mL) and extracted with EtOAc (2×50 mL). The combined organic layer was dried over Na₂SO₄, filtered, and concentrated under reduced pressure. The crude product was purified by silica gel (100-200 mesh) column chromatography [elution with 15% EtOAc in Hexane] to afford (S)-1-(4-(4,4,5,5-tetramethyl-1,3,2-dioxaborolan-2-yl)phenyl)pyrrolidin-3-ol as an off white solid (800 mg, Yield 45%).
TLC system: EtOAc/Hexane (30:70),
R_{f} value: ˜0.2;

LCMS (m/z): 290.36 (M+H)⁺.

¹H NMR (400 MHz, CDCl₃) δ: 7.46 (d, J=8.4 Hz, 2H), 6.47 (d, J=8.8 Hz, 2H), 4.96 (d, J=3.6 Hz, 1H), 4.39 (brs, 1H), 3.42-3.25 (m, 2H), 3.10-3.07 (m, 1H), 2.06-1.98 (m, 1H), 1.91-1.87 (m, 1H), 1.25 (s, 12H).

### Synthesis of diethyl(S)-(((6-fluoro-5-(4-(3-hydroxypyrrolidin-1-yl)phenyl)-1H-indol-3-yl)thio)methyl)phosphonate

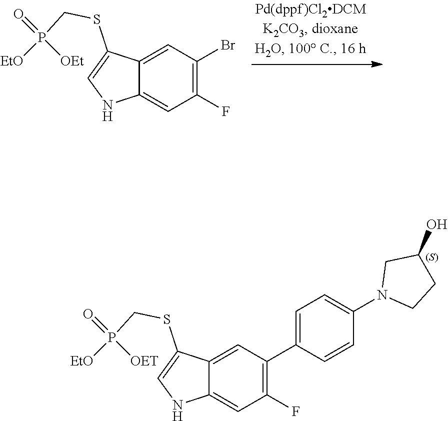

To a stirred solution of diethyl (((5-bromo-6-fluoro-1H-indol-3-yl)thio)methyl)phosphonate (200 mg, 0.51 mmol, 1.0 eq) in dioxane (3 mL), water (1 mL) was added (S)-1-(4-(4,4,5,5-tetramethyl-1,3,2-dioxaborolan-2-yl)phenyl)pyrrolidin-3-ol (231 mg, 0.82 mmol, 1.6 eq), K₂CO₃ (331 mg, 1.53 mmol, 3 eq). Reaction mixture was degassed with N₂ for 10 min then added Pd(dppf)Cl₂·DCM (40 mg, 0.05 mmol, 0.1 eq). The reaction mixture was stirred at 100° C. for 16 h. After completion of reaction by TLC, the reaction mixture was diluted with water (20 mL) and extracted with EtOAc (2×20 mL). The combined organic layer was dried over Na₂SO₄, filtered, and concentrated under reduced pressure. The crude product was purified by silica gel (100-200 mesh) column chromatography [elution with 2-4% MeOH in DCM] to afford diethyl(S)-(((6-fluoro-5-(4-(3-hydroxypyrrolidin-1-yl)phenyl)-1H-indol-3-yl)thio)methyl)phosphonate as brown liquid (150 mg, Yield 61%).
TLC system: EtOAc,
R_{f} value: ˜0.3;

LCMS (m/z): 479.38 (M+H)⁺.

¹H NMR (400 MHz, DMSO-d₆) δ: 11.44 (s, 1H), 7.61-7.58 (m, 2H), 7.39 (d, J=7.2 Hz, 2H), 7.25 (d, J=11.6 Hz, 1H), 6.60 (d, J=8.8 Hz, 2H), 4.98 (d, J=3.6 Hz, 1H), 4.42 (brs, 1H), 3.99-3.93 (m, 4H), 3.47-3.45 (m, 1H), 3.38-3.32 (m, 1H), 3.10-3.07 (m, 2H), 2.09-2.06 (m, 1H), 1.95-1.93 (m, 1H), 1.16 (t, J=7.2 Hz, 6H).

### Synthesis of diethyl(S)-(((6-fluoro-5-(4-(3-hydroxypyrrolidin-1-yl)phenyl)-1H-indol-3-yl)thio)methyl)phosphonic acid

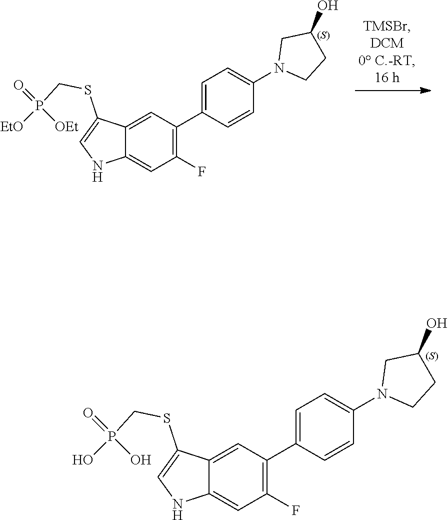

To a stirred solution of diethyl(S)-(((6-fluoro-5-(4-(3-hydroxypyrrolidin-1-yl)phenyl)-1H-indol-3-yl)thio)methyl)phosphonate (130 mg, 0.27 mmol, 1.0 eq) in DCM (3 mL) at 0° C., was added TMSBr (0.13 mL) and stirred at room temperature for 16 h. After completion of reaction by TLC, the reaction mixture was concentrated, diluted with water (2 mL), basified with 2M aq. NaOH solution, washed with EtOAc (2×10 mL). Followed by acidification of aq. layer with 2N HCl (pH˜2) and concentrated under reduced pressure. The crude product was purified by prep-HPLC to afford (S)-(((6-fluoro-5-(4-(3-hydroxypyrrolidin-1-yl)phenyl)-1H-indol-3-yl)thio)methyl)phosphonic acid as a white solid (17 mg, Yield 15%).
TLC system: MeOH/DCM (10:90),
R_{f} value: ^{˜}0.1;

LCMS (m/z): 421.15 (M−H)⁻.

¹H NMR (400 MHz, DMSO-d₆) δ: 11.29 (s, 1H), 7.57 (d, J=8.0 Hz, 1H), 7.48 (d, 1H), 7.35 (d, J=7.2 Hz, 2H), 7.21 (d, J=11.6 Hz, 1H), 6.55 (d, J=8.8 Hz, 2H), 4.94 (br, 1H), 4.40 (brs, 1H), 3.45-3.26 (m, 3H), 3.10 (d, J=9.2 Hz, 1H), 2.77 (d, J=14 Hz, 2H), 2.09-2.03 (m, 1H), 1.95-1.90 (m, 1H).

### Prep-HPLC Method:

COLUMN NAME: YMC TRIAT C₁₈ (250×19) mm 5 MICRON
Buffer: MP-(A): 0.1% FA IN WATER MP-(B): ACN:WATER (85:15) Gradient: (T/% B) 0/30, 12/60, 12.2/95, 16/95, 16.2/30, 19/30
Flow Rate 12 mL/min,
Solubility: water, acetonitrile, THF

### SYNTHETIC EXAMPLE 18

### Synthesis of Compound I-25

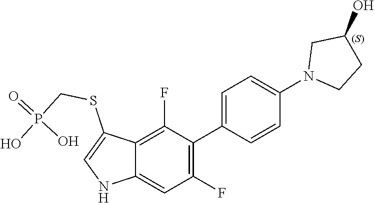

### Synthesis of 1,2-bis(5-bromo-4,6-difluoro-1H-indol-3-yl)disulfane

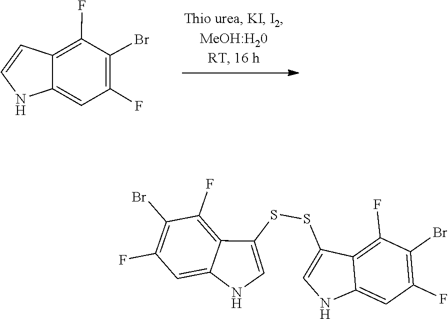 [SMILES: Fc1cc2[nH]ccc2c(F)c1Br ; Fc1cc2[nH]cc(SSc3c[nH]c4cc(F)c(Br)c(F)c34)c2c(F)c1Br]

To a stirred solution of 5-bromo-4,6-difluoro-1H-indole (2 g, 8.62 mmol, 1.0 eq) in MeOH (32 mL), water (8 mL) at room temperature, was added Thio urea (923 g, 12.9 mmol, 1.5 eq), I₂ (3.2 g, 12.9 mmol, 1.5 eq) and KI (2.14 g, 12.9 mmol, 1.5 eq), The reaction mixture was stirred at room temperature for 16 h. After completion of reaction by TLC, the reaction mixture was treated with 2N aq. NaOH solution (10 mL) & THF (10 mL) (clear solution) and stirred at 100° C. for 30 min. RM was concentrated, diluted with H₂O (50 mL), resulting precipitate was filtered, triturated with diethyl ether (20 mL) & filtered and dried under reduced pressure to afford 1,2-bis(5-bromo-4,6-difluoro-1H-indol-3-yl)disulfane as off white solid (480 mg, Yield 22%).
TLC system: MeOH in DCM (10:90),
R_{f} value: ˜0.3;

LCMS (m/z): 486.90 (M+H)⁺.

### Synthesis of diethyl (((5-bromo-4,6-difluoro-1H-indol-3-yl)thio)methyl)phosphonate

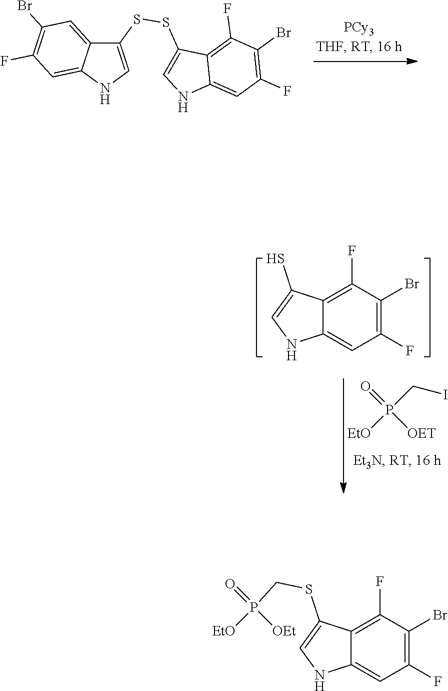

To a stirred solution of 1,2-bis(5-bromo-4,6-difluoro-1H-indol-3-yl)disulfane (600 mg, 1.14 mmol, 1.0 eq) in THF (6 mL), was added PCy₃ (641 mg, 2.28 mmol, 2.0 eq) and stirred at room temperature for 16 h. Later, added diethyl (iodomethyl)phosphonate (637 mg, 2.28 mmol, 2.0 eq) and TEA (0.33 mL, 2.28 mmol, 2 eq). The reaction mixture was stirred at room temperature for 16 h. After completion of reaction by TLC, the reaction mixture was quenched with water (40 mL) and extracted with EtOAc (2×20 mL). The combined organic layer was dried over Na₂SO₄, filtered, and concentrated under reduced pressure. The crude product was purified by silica gel (100-200 mesh) column chromatography [elution with 0.5-1% MeOH in DCM] to afford diethyl (((5-bromo-4,6-difluoro-1H-indol-3-yl)thio)methyl)phosphonate as a pale brown oil (0.5 g, Yield 95%).
TLC system: MeOH in DCM (10:90),
R_{f} value: ˜0.4;

LCMS (m/z): 414 (M+H)⁺.

### Synthesis of diethyl (diethyl(S)-(((4,6-difluoro-5-(4-(3-hydroxypyrrolidin-1-yl)phenyl)-1H-indol-3-yl)thio)methyl)phosphonate

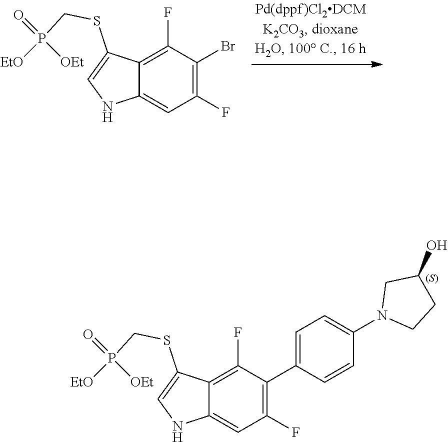

To a stirred solution of diethyl (((5-bromo-4,6-difluoro-1H-indol-3-yl)thio)methyl)phosphonate (400 mg, 0.97 mmol, 1.0 eq) in dioxane (8 mL), water (3 mL), was added (S)-1-(4-(4,4,5,5-tetramethyl-1,3,2-dioxaborolan-2-yl)phenyl)pyrrolidin-3-ol (447 mg, 1.54 mmol, 1.6 eq), K₂CO₃ (400 mg, 2.91 mmol, 3 eq). Reaction mixture was degassed with N₂ for 10 min then added Pd(dppf)Cl₂·DCM (78 mg, 0.096 mmol, 0.1 eq). The reaction mixture was stirred at 100° C. for 16 h. After completion of reaction by TLC, diluted with water (50 mL) and extracted with EtOAc (2×40 mL). The combined organic layer was dried over Na₂SO₄, filtered, and concentrated under reduced pressure. The crude product was purified by silica gel (100-200 mesh) column chromatography [elution with 2-4% MeOH in DCM] to afford diethyl(S)-(((4,6-difluoro-5-(4-(3-hydroxypyrrolidin-1-yl)phenyl)-1H-indol-3-yl)thio)methyl)phosphonate as brown liquid (300 mg, Yield: 62%),
TLC system: EtOAc,
R_{f} value: ˜0.2;

LCMS (m/z): 497.28 (M+H)⁺.

### Synthesis of(S)-(((4,6-difluoro-5-(4-(3-hydroxypyrrolidin-1-yl)phenyl)-1H-indol-3-yl)thio)methyl)phosphonic acid

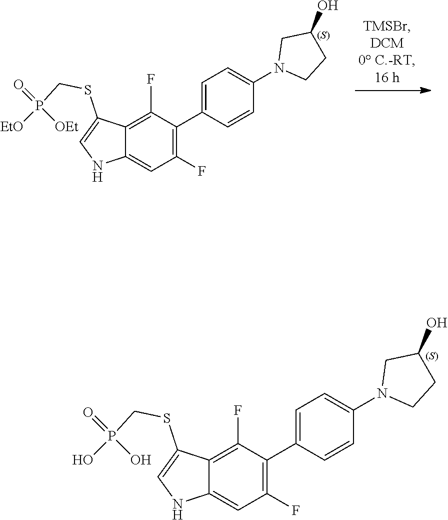

To a stirred solution of diethyl(S)-(((4,6-difluoro-5-(4-(3-hydroxypyrrolidin-1-yl)phenyl)-1H-indol-3-yl)thio)methyl)phosphonate (130 mg, 0.26 mmol, 1.0 eq) in DCM (3 mL) at 0° C. was added TMSBr (0.13 mL) and stirred at room temperature for 16 h. After completion of reaction by TLC, volatiles removed under vacuum, diluted with water (10 mL), basified with 2M aq. NaOH solution, washed with EtOAc (2×10 mL). Followed by acidification of aq. layer with 2N HCl (pH˜2), evaporated and purified by prep-HPLC to afford (S)-(((4,6-difluoro-5-(4-(3-hydroxypyrrolidin-1-yl)phenyl)-1H-indol-3-yl)thio)methyl)phosphonic acid as a white solid (31 mg, Yield 26%).
TLC system: MeOH/DCM (10:90),
R_{f} value: ^{˜}0.1;

LCMS (m/z): 441.2 (M+H)⁺.

¹H NMR (400 MHz, DMSO-d₆) δ: 11.54 (s, 1H), 7.38 (s, 1H), 7.21 (d, J=8.4 Hz, 2H), 7.07 (d, J=10 Hz, 1H), 6.57 (d, J=8.4 Hz, 2H), 5.03 (brs, 1H), 4.41 (m, 1H), 3.35-3.31 (m, 2H), 3.13-3.10 (m, 2H), 2.81 (d, J=14 Hz, 2H), 2.08-2.04 (m, 1H), 1.95-1.91 (m, 1H).

### Prep-HPLC Method:

Column GEMINI C-18 (250×21.2) mm 5mic
Buffer MP-(A): 0.1% FA IN WATER MP-(B): ACN Gradient (T/% B) 0/25, 15/50, 25/95
Flow Rate 13 mL/min
Solubility: acetonitrile, water

### SYNTHETIC EXAMPLE 19

### Synthesis of Compound I-27

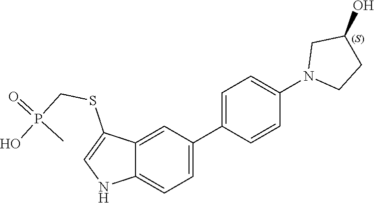

### Synthesis of 1,2-bis(5-bromo-1H-indol-3-yl)disulfane

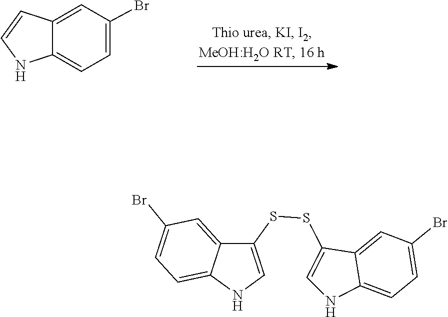 [SMILES: Brc1ccc2[nH]ccc2c1 ; Brc1ccc2[nH]cc(SSc3c[nH]c4ccc(Br)cc34)c2c1]

To a stirred solution of 5-bromo-1H-indole (5 g, 25.5 mmol, 1.0 eq) in MeOH (80 mL) & Water (20 mL) at room temperature, was added thio urea (1.93 g, 25.5 mmol, 1.0 eq), I₂ (6.32 g, 25.5 mmol, 1.0 eq) and KI (4.15 g, 25.5 mmol, 1.0 eq), The reaction mixture was stirred at room temperature for 16 h. After completion of reaction by TLC, the reaction mixture was treated with 2N NaOH solution (10 mL) & THF (10 mL) (clear solution) and stirred at 100° C. for 30 min. RM was concentrated under reduced pressure. The reaction mixture was diluted with H₂O (50 mL), resulting precipitate was filtered, triturated with diethyl ether (20 mL) and dried under vacuum to afford 1,2-bis(5-bromo-1H-indol-3-yl)disulfane as off white solid (2.2 g, Yield 38%).
TLC system: EtOAc/Hexane (50:50) or MeOH in DCM (10:90),
R_{f} value: ˜0.3;

LCMS (m/z): 80%, 450.87 (M−H)⁺.

### Synthesis of (ethoxy(methyl)phosphoryl)methyl 4-methylbenzenesulfonate (Int-04)

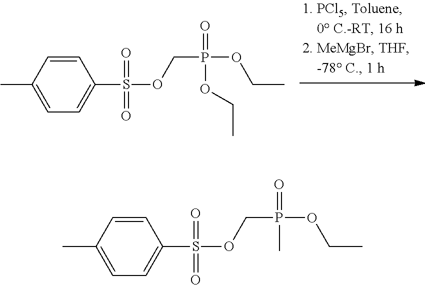

To a stirred solution of (diethoxyphosphoryl)methyl 4-methylbenzenesulfonate (500 mg, 1.55 mmol, 1.0 eq) in dry toluene (3 mL) at 0° C. was added PCl₅ (484 mg, 2.32 mmol, 1.5 eq) and stirred for 20 min, later allowed to room temperature, stirred for 16 h. The reaction mixture was concentrated, diluted with dry THF (3 mL), cooled to −78° C. and added 3M CH₃MgBr (0.5 mL, 1.55 mmol, 1.0 eq.). Reaction mixture was stirred at same temperature for 1 h. After completion of reaction by TLC, quenched with saturated ammonium chloride solution and extracted with EtOAc (2×20 mL). Organic layer was dried over Na₂SO₄ and concentrated under reduced pressure to afford crude product. The crude product was purified by silica gel (100-200 mesh) column chromatography [elution with 2-4% MeOH in DCM] to afford (ethoxy(methyl)phosphoryl)methyl 4-methylbenzenesulfonate as white solid (250 mg, Yield 55%).
TLC system: MeOH/DCM (10:90),
R_{f} value: ^{˜}0.3;

LCMS (m/z): 293.10 (M+H)⁺.

¹H NMR (400 MHz, DMSO-d6) δ: 7.84 (d, J=8.0 Hz, 2H), 7.51 (d, J=8.0 Hz, 2H), 4.36-4.27 (m, 2H), 3.98-3.82 (m, 2H), 2.44 (s, 3H), 1.43 (d, J=14.8 Hz, 3H), 1.16 (t, J=7.2 Hz, 3H),

### Synthesis of ethyl (((5-bromo-1H-indol-3-yl)thio)methyl)(methyl)phosphinate

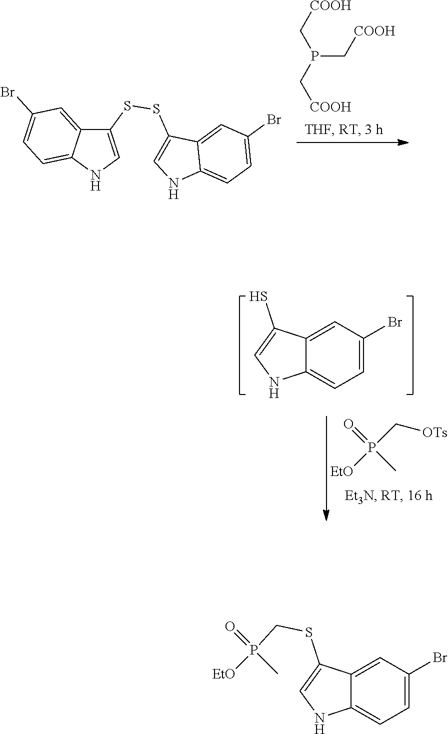

To a stirred solution of 1,2-bis(5-bromo-1H-indol-3-yl)disulfane (2 g, 4.42 mmol, 1.0 eq) in THF (20 mL), was added 2,2′,2″-phosphanetriyltriacetic acid (3 g, 10.6 mmol, 2.4 eq) at room temperature, The reaction mixture was stirred at room temperature for 16 h. After completion of reaction by LC-MS, added (ethoxy(methyl)phosphoryl)methyl 4-methylbenzenesulfonate (2.4 g, 8.84 mmol, 2.0 eq) and TEA (3.18 mL, 22.1 mmol, 5 eq). The reaction mixture was stirred at room temperature for 16 h. After completion of reaction by TLC, the reaction mixture was quenched with water (50 mL) and extracted with EtOAc (2×50 mL). The combined organic layer was dried over Na₂SO₄, filtered and concentrated under reduced pressure to afford crude product. The crude product was purified by silica gel (100-200 mesh) column chromatography [elution with 0.5-1% MeOH in DCM] to afford ethyl (((5-bromo-1H-indol-3-yl)thio)methyl)(methyl)phosphinate as a pale brown oil (450 mg, Yield 30%).
TLC system: EtOAc/Hexane (50:50),
R_{f} value: ˜0.4;

LCMS (m/z): 85%, 346.05 (M−H)⁻.

¹H NMR (400 MHz, CDCl₃) δ: 8.61 (brs, 1H), 7.87 (s, 1H), 7.46 (d, J=2.8 Hz, 1H), 7.33 (dd, J=8.4, 1.6 Hz, 1H), 7.19-7.17 (m, 1H), 4.08-4.03 (m, 1H), 3.96-3.88 (m, 1H), 3.04-2.91 (m, 2H), 1.26 (t, J=7.2 Hz, 3H). P—CH₃ protons might be merging with solvents peak.

### Synthesis of ethyl (((5-(4-((S)-3-hydroxypyrrolidin-1-yl)phenyl)-1H-indol-3-yl)thio)methyl)(methyl)phosphinate

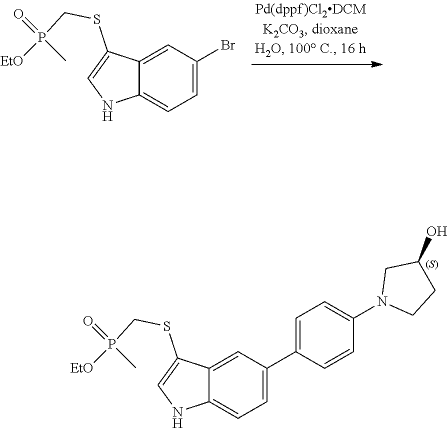

To a stirred solution of ethyl (((5-bromo-1H-indol-3-yl)thio)methyl)(methyl)phosphinate (450 mg, 1.29 mmol, 1.0 eq) in Dioxane (7 mL), water (2 mL), was added (S)-1-(4-(4,4,5,5-tetramethyl-1,3,2-dioxaborolan-2-yl)phenyl)pyrrolidin-3-ol (448 mg, 1.55 mmol, 1.2 eq), K₂CO₃ (356 mg, 2.58 mmol, 2 eq). Reaction mixture was degassed with N₂ for 10 min then added Pd(dppf)Cl₂·DCM (105 mg, 0.13 mmol, 0.1 eq). The reaction mixture was stirred at 100° C. for 16 h. After completion of reaction by TLC, the reaction mixture was diluted with water (20 mL) and extracted with EtOAc (2×20 mL). The combined organic layer was dried over Na₂SO₄, filtered and concentrated under reduced pressure to afford crude product. The crude product was purified by silica gel (100-200 mesh) column chromatography [elution with 2-4% MeOH in DCM] to afford ethyl (((5-(4-((S)-3-hydroxypyrrolidin-1-yl)phenyl)-1H-indol-3-yl)thio)methyl)(methyl)phosphinate as a brown gummy solid (350 mg, Yield: 63%).
TLC system: EtOAc
R_{f} value: ˜0.3;

LCMS (m/z): 95%, 431.31 (M+H)⁺.

### Synthesis of diethyl ((((5-(4-((S)-3-hydroxypyrrolidin-1-yl)phenyl)-1H-indol-3-yl)thio)methyl)(methyl)phosphinic acid

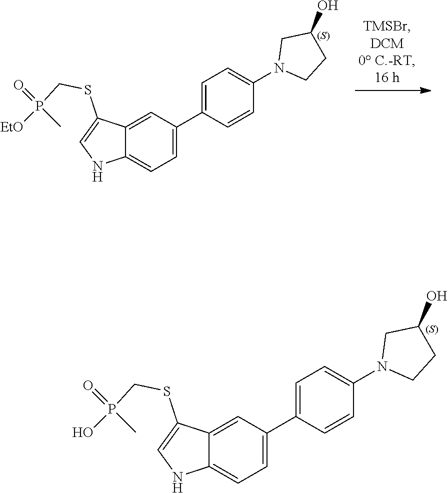

To a stirred solution of diethyl ethyl (((5-(4-((S)-3-hydroxypyrrolidin-1-yl)phenyl)-1H-indol-3-yl)thio)methyl)(methyl)phosphinate (350 mg, 5.4 mmol, 1.0 eq) in DCM (3 mL) at 0° C. was added TMSBr (0.35 mL) and stirred at room temperature for 16 h. After completion of reaction by TLC, the reaction mixture was concentrated, diluted with water (10 mL), basified with 2M NaOH solution, washed with EtOAc (2×10 mL). Aqueous layer was acidified with 2N HCl (pH˜2) and concentrated under reduced pressure to afford crude product. The crude product was purified by prep-HPLC provided (((5-(4-((S)-3-hydroxypyrrolidin-1-yl)phenyl)-1H-indol-3-yl)thio)methyl)(methyl)phosphinic acid as Off white solid (29 mg, Yield 8.5%).
TLC system: MeOH/DCM (10:90),
R_{f} value: ^{˜}0;

LCMS (m/z): 403.20 (M+H)⁺.

¹H NMR (400 MHz, DMSO-d6, D2O ex) 8:11.33 (s, 1H), 7.75 (s, 1H), 7.54 (d, J=2.4 Hz, 1H), 7.51 (d, J=8.4 Hz, 2H), 7.41 (d, J=8.4 Hz, 1H), 7.38-7.35 (m, 1H), 6.58 (d, J=8.4 Hz, 2H), 4.97 (brs, 1H), 4.41 (brs, 1H), 3.46-3.37 (m, 3H), 3.11-3.09 (m, 1H), 2.89 (d, J=11.2 Hz, 2H), 2.07-2.01 (m, 1H), 1.92-1.88 (m, 1H), 1.32 (d, J=14.4 Hz, 3H).

### Prep Conditions:

Crude purity (HPLC/LCMS) 67
Column GEMINI (250×21.2) mm 5mic Buffer MP-(A): 0.1% FA IN WATER MP-(B): CAN Gradient (T/% B) 0/20, 15/55, 25/95
Flow Rate 14 mL/min
Solubility: acetonitrile, THF

### SYNTHETIC EXAMPLE 20

### Synthesis of Compound I-28

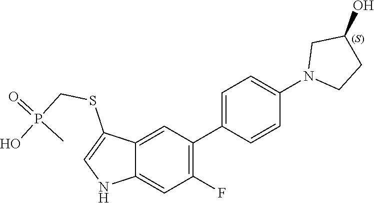

### Synthesis of ethyl (((5-bromo-6-fluoro-1H-indol-3-yl)thio)methyl)(methyl)phosphinate

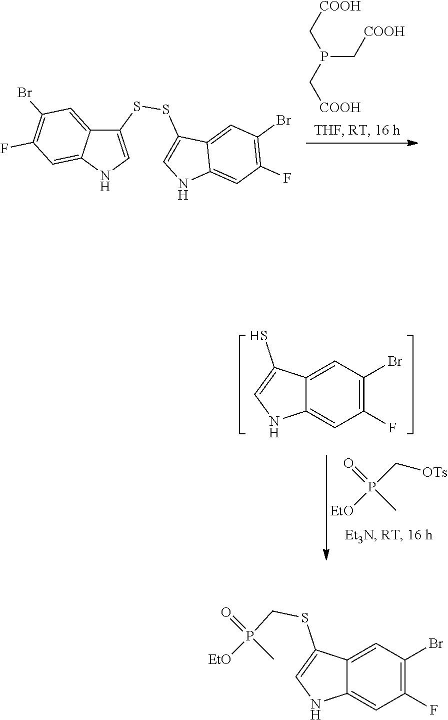

To a stirred solution of 1,2-bis(5-bromo-6-fluoro-1H-indol-3-yl)disulfane (550 mg, 1.12 mmol, 1.0 eq) in THF (10 mL), was added 2,2′,2″-phosphanetriyltriacetic acid (644 mg, 2.25 mmol, 2.0 eq) and stirred at room temperature for 16 h. After completion of reaction by TLC, added (ethoxy(methyl)phosphoryl)methyl 4-methylbenzenesulfonate (658 mg, 2.25 mmol, 2.0 eq), TEA (0.8 mL, 5.56 mmol, 5 eq) and stirred at room temperature for 16 h. After completion of reaction by TLC, the reaction mixture was quenched with water (40 mL) and extracted with EtOAc (3×20 mL). The combined organic layer was dried over Na₂SO₄, filtered, and concentrated under reduced pressure to afford crude product. The crude product was purified by silica gel (100-200 mesh) column chromatography [elution with 2-5% MeOH in DCM] to afford ethyl (((5-bromo-6-fluoro-1H-indol-3-yl)thio)methyl)(methyl)phosphinate as a pale brown oil (0.5 g, Yield 49%).
TLC system: EtOAc/Hexane (50:50),
R_{f} value: ˜0.3;

¹H NMR (400 MHz, CDCl₃) δ: 9.08 (s, 1H), 7.87 (d, J=6.4 Hz, 1H), 7.42 (d, J=2.4 Hz, 1H), 7.11 (d, J=8.8 Hz, 1H), 4.11-3.94 (m, 2H), 2.95 (d, J=10.4 Hz, 2H), 1.60 (d, 3H), 1.27 (t, J=7.2 Hz, 3H).

### Synthesis of ethyl (((6-fluoro-5-(4-((S)-3-hydroxypyrrolidin-1-yl)phenyl)-1H-indol-3-yl)thio)methyl)(methyl)phosphinate

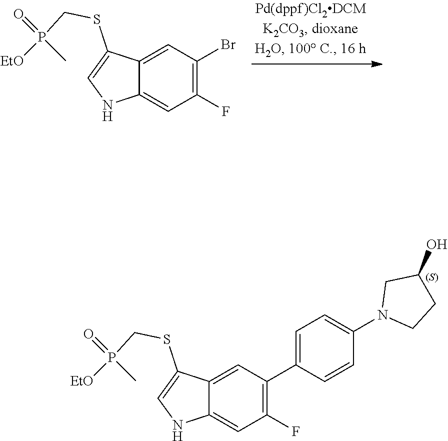

To a stirred solution of ethyl (((5-bromo-6-fluoro-1H-indol-3-yl)thio)methyl)(methyl)phosphinate (500 mg, 1.37 mmol, 1.0 eq) in dioxane (8 mL), water (2 mL) was added (S)-1-(4-(4,4,5,5-tetramethyl-1,3,2-dioxaborolan-2-yl)phenyl)pyrrolidin-3-ol (475 mg, 1.64 mmol, 1.2 eq), K₂CO₃ (378 mg, 2.74 mmol, 2 eq). Reaction mixture was degassed with N₂ for 10 min then added Pd(dppf)Cl₂·DCM (111 mg, 0.137 mmol, 0.1 eq) and stirred at 100° C. for 16 h. After completion of reaction by TLC, diluted with water (20 mL) and extracted with EtOAc (2×20 mL). The combined organic layer was dried over Na₂SO₄, filtered, and concentrated under reduced pressure to afford crude product. The crude product was purified by silica gel (100-200 mesh) column chromatography [elution with 2-4% MeOH in DCM] to afford ethyl (((6-fluoro-5-(4-((S)-3-hydroxypyrrolidin-1-yl)phenyl)-1H-indol-3-yl)thio)methyl)(methyl)phosphinate as brown solid (400 mg, Yield 65%).
TLC system: 10% MeOH in DCM,
R_{f} value: ˜0.4;

LCMS (m/z): 449.32 (M+H)⁺.

¹H NMR (400 MHz, CDCl3) δ: 8.55 (brs, 1H), 7.68 (d, J=7.6 Hz, 1H), 7.52-7.48 (m, 2H), 7.42 (d, J=2.4 Hz, 1H), 7.14 (d, J=10.8 Hz, 1H), 6.72-6.70 (m, 2H), 4.65-4.64 (m, 1H), 4.10-4.02 (m, 1H), 3.93-3.89 (m, 1H), 3.62-3.55 (m, 2H), 3.44-3.35 (m, 2H), 3.05-2.96 (m, 2H), 2.24-2.12 (m, 2H), 1.56 (d, 3H), 1.28-1.26 (t, 3H).

### Synthesis of (((6-fluoro-5-(4-((S)-3-hydroxypyrrolidin-1-yl)phenyl)-1H-indol-3-yl)thio)methyl)(methyl)phosphinic acid

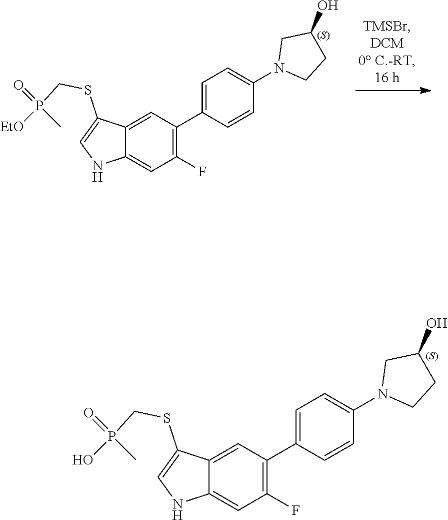

To a stirred solution of ethyl (((6-fluoro-5-(4-((S)-3-hydroxypyrrolidin-1-yl)phenyl)-1H-indol-3-yl)thio)methyl)(methyl)phosphinate (200 mg, 0.44 mmol, 1.0 eq) in DCM (6 mL) at 0° C. was added TMSBr (0.2 mL) and stirred at room temperature for 16 h. After completion of reaction by TLC, the reaction mixture was concentrated, diluted with water (10 mL), basified with 2M NaOH solution and washed with EtOAc (2×10 mL). Aqueous layer was acidified with 2N HCl (pH˜2) and concentrated under reduced pressure. The crude product was purified by prep-HPLC to afford (((6-fluoro-5-(4-((S)-3-hydroxypyrrolidin-1-yl)phenyl)-1H-indol-3-yl)thio)methyl)(methyl)phosphinic acid as white solid (27 mg, Yield 14%).
TLC system: MeOH/DCM (10:90),
R_{f} value: ^{˜}0;

LCMS (m/z): 421.1 (M+H)⁺.

¹H NMR (400 MHz, DMSO-d₆) δ: 11.39 (s, 1H), 7.59-7.56 (m, 2H), 7.38 (d, J=7.2 Hz, 2H), 7.24 (d, J=11.2 Hz, 1H), 6.59 (d, J=8.8 Hz, 2H), 4.97 (brs, 1H), 4.42 (brs, 1H), 3.46-3.28 (m, 3H), 3.11 (d, J=8.8 Hz, 1H), 2.89 (d, J=11.2 Hz, 2H), 2.08-2.04 (m, 1H), 1.93-1.88 (m, 1H), 1.32 (d, J=14.4 Hz, 3H).

### Prep-HPLC Method:

Crude purity (HPLC/LCMS) 63%
Column GEMINI C-18 (250× 21.2) mm 5mic
Buffer MP-(A): 0.1% FA IN WATER MP-(B): ACN Gradient (T/% B) 0/20, 15/55, 25/95
Flow Rate 15 mL/min
Solubility: water, acetonitrile, THF

### SYNTHETIC EXAMPLE 21

### Synthesis of Compound I-29

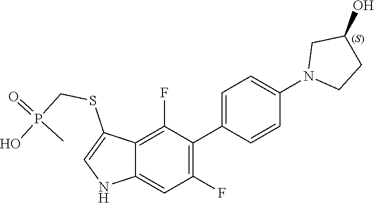

### Synthesis of ethyl (((5-bromo-4,6-difluoro-1H-indol-3-yl)thio)methyl)(methyl)phosphinate

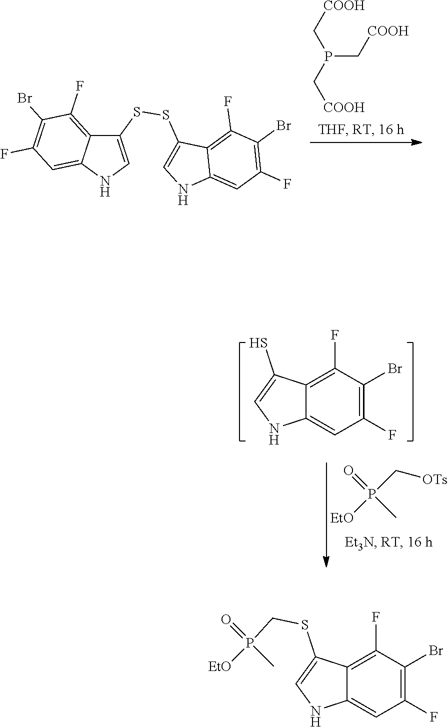

To a stirred solution of 1,2-bis(5-bromo-4,6-difluoro-1H-indol-3-yl)disulfane (1.5 g, 2.86 mmol, 1.0 eq) in THF (15 mL), was added 2,2′,2″-phosphanetriyltriacetic acid (2 g, 6.87 mmol, 2.45 eq) and stirred at room temperature for 16 h. After completion of reaction by TLC, & LC-MS, LC-MS indicated 75% m/z, then added (ethoxy(methyl)phosphoryl)methyl 4-methylbenzenesulfonate (1.66 g, 5.72 mmol, 2.0 eq), TEA (3.06 mL, 21.5 mmol, 7.5 eq) and stirred at room temperature for 16 h. After completion of reaction by TLC, the reaction mixture was quenched with water (70 mL) and extracted with EtOAc (3×50 mL). The combined organic layer was dried over Na₂SO₄, filtered and concentrated under reduced pressure to afford crude product. The crude product was purified by silica gel (100-200 mesh) column chromatography [elution with 2-5% MeOH in DCM] to afford ethyl (((5-bromo-4,6-difluoro-1H-indol-3-yl)thio)methyl)(methyl)phosphinate as a pale brown gummy solid (0.5 g, Yield 45%).
TLC system: EtOAc/Hexane (50:50),
R_{f} value: ˜0.3;

LCMS (m/z): 384.06 (M+H)⁺.

### Synthesis of ethyl (((4,6-difluoro-5-(4-((S)-3-hydroxypyrrolidin-1-yl)phenyl)-1H-indol-3-yl)thio)methyl)(methyl)phosphinate

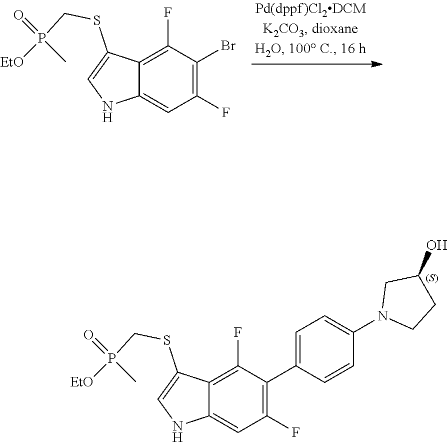

To a stirred solution of ethyl (((5-bromo-1H-indol-3-yl)thio)methyl)(methyl)phosphinate (500 mg, 1.30 mmol, 1.0 eq) in Dioxane (8 mL), water (2 mL) was added (S)-1-(4-(4,4,5,5-tetramethyl-1,3,2-dioxaborolan-2-yl)phenyl)pyrrolidin-3-ol (452 mg, 1.56 mmol, 1.2 eq), K₂CO₃ (360 mg, 2.61 mmol, 2 eq). Reaction mixture was degassed with N₂ for 10 min then added Pd(dppf)Cl2·DCM (106 mg, 0.13 mmol, 0.1 eq). The reaction mixture was stirred at 100° C. for 16 h. After completion of reaction by TLC, the reaction mixture was diluted with water (20 mL) and extracted with EtOAc (2×20 mL). The combined organic layer was dried over Na₂SO₄, filtered and concentrated under reduced pressure to afford crude product. The crude product was purified by silica gel (100-200 mesh) column chromatography [elution with 2-4% MeOH in DCM] to afford ethyl (((4,6-difluoro-5-(4-((S)-3-hydroxypyrrolidin-1-yl)phenyl)-1H-indol-3-yl)thio)methyl)(methyl)phosphinate as brick red solid (350 mg, Yield 57%),
TLC system: 10% MeOH in DCM,
R_{f} value: ˜0.5;

LCMS (m/z): 467.37 (M+H)⁺.

¹H NMR (400 MHz, DMSO-d₆) δ: 11.40 (s, 1H), 7.77 (s, 1H), 7.61 (d, J=2.4 Hz, 1H), 7.55 (d, J=8.8 Hz, 2H), 6.63 (d, J=8.4 Hz, 2H), 4.98 (d, J=3.6 Hz, 1H), 4.45 (brs, 1H), 3.97-3.81 (m, 2H), 3.50-3.46 (m, 1H), 3.40-3.37 (m, 1H), 3.15-3.08 (m, 3H), 2.10-2.06 (m, 1H), 1.98-1.90 (m, 1H), 1.49 (d, J=14.4 Hz, 3H), 1.15 (t, J=6.8 Hz, 3H).

### Synthesis of (((4,6-difluoro-5-(4-((S)-3-hydroxypyrrolidin-1-yl)phenyl)-1H-indol-3-yl)thio)methyl)(methyl)phosphinic acid

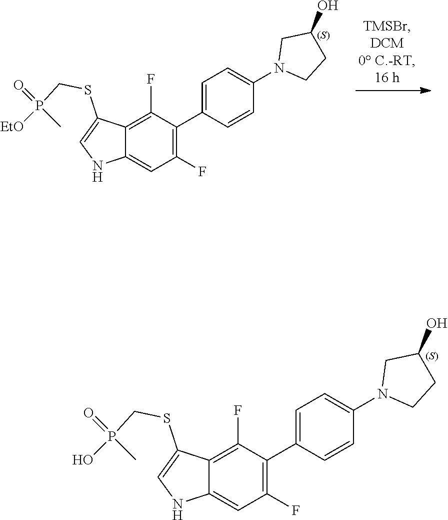

To a stirred solution of ethyl (((4,6-difluoro-5-(4-((S)-3-hydroxypyrrolidin-1-yl)phenyl)-1H-indol-3-yl)thio)methyl)(methyl)phosphinate (350 mg, 0.93 mmol, 1.0 eq) in DCM (10 mL) at 0° C. was added TMSBr (0.35 mL) and stirred at room temperature for 16 h. After completion of reaction by TLC, the reaction mixture was concentrated, diluted with water (10 mL), basified with 2M NaOH solution and washed with EtOAc (2×10 mL). Aqueous layer was acidified with 2N HCl (pH˜2) and concentrated under reduced pressure to afford crude product. The crude product was purified by prep-HPLC to afford (((4,6-difluoro-5-(4-((S)-3-hydroxypyrrolidin-1-yl)phenyl)-1H-indol-3-yl)thio)methyl)(methyl)phosphinic acid as white solid (64 mg, Yield 18%).
TLC system: MeOH/DCM (10:90),
R_{f} value: ^{˜}0;

LCMS (m/z): 437.15 (M−H)⁻.

¹H NMR (400 MHz, DMSO-d₆) δ: 11.62 (s, 1H), 7.52 (d, J=2.4 Hz, 1H), 7.24 (d, J=8.4 Hz, 2H), 7.13 (d, J=10 Hz, 1H), 6.60 (d, J=8.8 Hz, 2H), 4.96 (br, 1H), 4.43 (brs, 1H), 3.47-3.27 (m, 3H), 3.13-3.05 (m, 1H), 2.95 (d, J=12 Hz, 2H), 2.09-2.02 (m, 1H), 1.94-1.89 (m, 1H), 1.34 (d, J=14.4 Hz, 3H).

### Prep Conditions:

Crude purity (HPLC/LCMS) 55% Column GEMINI C-18 (250× 21.2) mm 5mic
Buffer MP-(A): 0.1% FA IN WATER MP-(B): ACN Gradient (T/% B) 0/30, 15/55, 25/95
Flow Rate 13 mL/min
Solubility: THF, water, acetonitrile

### SYNTHETIC EXAMPLE 22

### Synthesis of Compound I-35

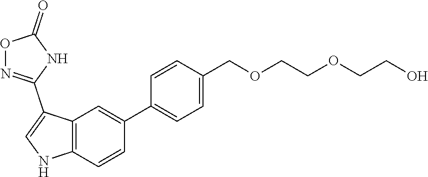 [SMILES: O=c1[nH]c(-c2c[nH]c3ccc(-c4ccc(COCCOCCO)cc4)cc23)no1]

### Synthesis of 3-(5-(4-((2-(2-hydroxyethoxy)ethoxy)methyl)phenyl)-1-((2-(trimethylsilyl)ethoxy)methyl)-1H-indol-3-yl)-1,2,4-oxadiazol-5(4H)-one

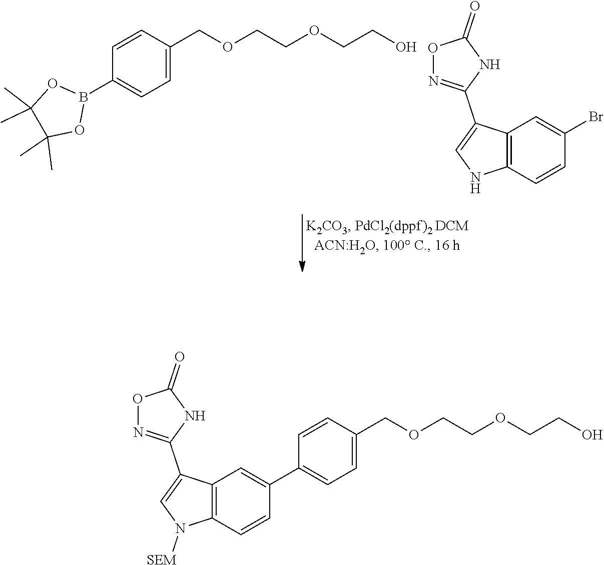

In a sealed tube, to a degassed solution of 3-(5-bromo-1H-indol-3-yl)-1,2,4-oxadiazol-5(4H)-one (300 mg, 0.73 mmol, 1.0 eq) and 2-(2-((4-(4,4,5,5-tetramethyl-1,3,2-dioxaborolan-2-yl)benzyl)oxy)ethoxy)ethan-1-ol (590 mg, 1.83 mmol, 2.5 eq) in ACN:H₂O (1:1, 6 mL) at room temperature, was added K₂CO₃ (303 mg, 2.20 mmol, 3 eq) followed by Pd(dppf)Cl₂·DCM (30 mg, 0.036 mmol, 0.05 eq) and stirred at 100° C. for 16 h. After completion of reaction by TLC, the reaction mixture was diluted with water (20 mL) and washed with 10% MeOH/DCM (2×20 mL). Aqueous layer concentrated and purified silica-gel column chromatography [elution with 30% Ethyl acetate in hexane] to afford 3-(5-(4-((2-(2-hydroxyethoxy)ethoxy)methyl)phenyl)-1-((2-(trimethylsilyl)ethoxy)methyl)-1H-indol-3-yl)-1,2,4-oxadiazol-5(4H)-one as an off white solid (150 mg, Yield: 39%).
TLC system: EtOAc (100%)
R_{f} value: ˜0.2

LCMS (m/z): 524.27 (M−H)⁺

### Synthesis of 3-(5-(4-((2-(2-hydroxyethoxy)ethoxy)methyl)phenyl)-1H-indol-3-yl)-1,2,4-oxadiazol-5(4H)-one

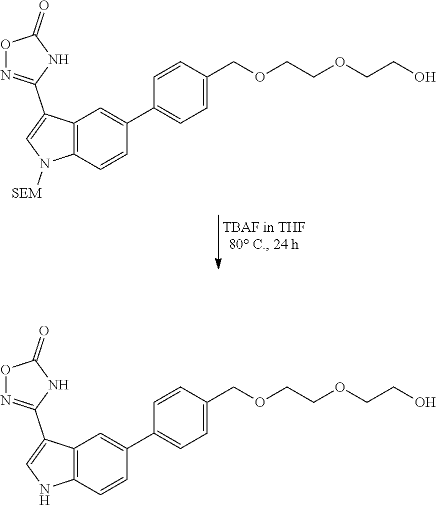 [SMILES: C[Si](C)(C)CCOCn1cc(-c2noc(=O)[nH]2)c2cc(-c3ccc(COCCOCCO)cc3)ccc21 ; O=c1[nH]c(-c2c[nH]c3ccc(-c4ccc(COCCOCCO)cc4)cc23)no1]

To a stirred solution of 3-(5-(4-((2-(2-hydroxyethoxy)ethoxy)methyl)phenyl)-1-((2-(trimethylsilyl)ethoxy)methyl)-1H-indol-3-yl)-1,2,4-oxadiazol-5(4H)-one (130 mg, 0.24 mmol, 1.0 eq) in THF (1.3 mL, 10 Vol) at 0° C., was added TBAF (4 mL) and stirred at 80° C. for 24 h. After completion of reaction by TLC, volatiles removed, washed with pentane (10 mL) and purified by RP column [elution with 40-45% ACN:Water, (0.1% FA buffer)]. After evaporation, triturated with pentane, ether & acetonitrile followed by lyophilization afforded 3-(5-(4-((2-(2-hydroxyethoxy)ethoxy)methyl)phenyl)-1H-indol-3-yl)-1,2,4-oxadiazol-5(4H)-one as off white solid (42 mg, 43%).
TLC system: 10% MeOH in DCM
R_{f} value: ˜0.2

LCMS (m/z): 394.1 (M−H)⁻

¹H NMR (400 MHz, DMSO-d₆) δ: 12.56 (brs, 1H), 11.97 (s, 1H), 8.14 (s, 1H), 7.99 (d, J=2.8 Hz, 1H), 7.66-7.55 (m, 4H), 7.42 (d, J=8.0 Hz, 2H), 4.59 (br, s, 1H), 4.54 (s, 2H), 3.59 (s, 4H), 3.51-3.44 (m, 4H).

### SYNTHETIC EXAMPLE 23

### Synthesis of Compound I-36

### Synthesis of 3-(6-fluoro-5-(4-((2-(2-hydroxyethoxy)ethoxy)methyl)phenyl)-1H-indol-3-yl)-1,2,4-oxadiazol-5(4H)-one

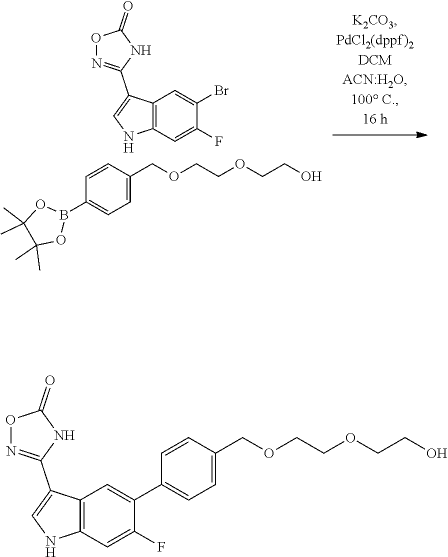

In a sealed tube, to a degassed solution of 3-(5-bromo-6-fluoro-1H-indol-3-yl)-1,2,4-oxadiazol-5(4H)-one (200 mg, 0.67 mmol, 1.0 eq) and 2-(2-((4-(4,4,5,5-tetramethyl-1,3,2-dioxaborolan-2-yl)benzyl)oxy)ethoxy)ethan-1-ol (542 mg, 1.68 mmol, 2.5 eq) in ACN:H₂O (1:1, 4 mL) at room temperature, was added K₂CO₃ (278 mg, 2.02 mmol, 3 eq) followed by Pd(dppf)Cl₂·DCM (28 mg, 0.033 mmol, 0.05 eq) and stirred at 100° C. for 16 h. After completion of reaction by TLC, the reaction mixture was diluted with water (20 mL) and washed with 10% MeOH/DCM (2×20 mL). Aqueous layer evaporated and purified by C₁₈ reverse phase column chromatography [elution with 40-45% ACN:Water, (0.1% FA Buffer)] to afford 3-(6-fluoro-5-(4-((2-(2-hydroxyethoxy)ethoxy)methyl)phenyl)-1H-indol-3-yl)-1,2,4-oxadiazol-5(4H)-one as an off white solid (66.7 mg, Yield: 24%).
TLC system: 10% MeOH in DCM,
R_{f} value: ˜0.1;

LCMS (m/z): 412.1 (M−H)⁻

¹H NMR (400 MHz, DMSO-d₆) δ: 12.66 (brs, 1H), 12.00 (s, 1H), 8.00 (d, J=2.8 Hz, 1H), 7.93 (d, J=7.6 Hz, 1H), 7.55-7.53 (m, 2H), 7.49-7.43 (m, 3H), 4.60 (t, J=4.8 Hz, 1H). 4.56 (s, 2H), 3.60 (s, 4H), 3.53-3.49 (m, 2H), 3.46-3.43 (m, 2H).

### SYNTHETIC EXAMPLE 24

### Synthesis of Compound I-37

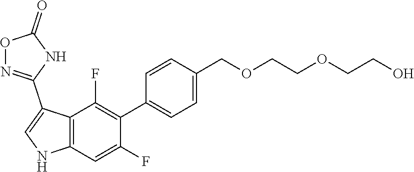 [SMILES: O=c1[nH]c(-c2c[nH]c3cc(F)c(-c4ccc(COCCOCCO)cc4)c(F)c23)no1]

### Synthesis of 2-(2-((4-bromobenzyl)oxy)ethoxy)ethan-1-ol

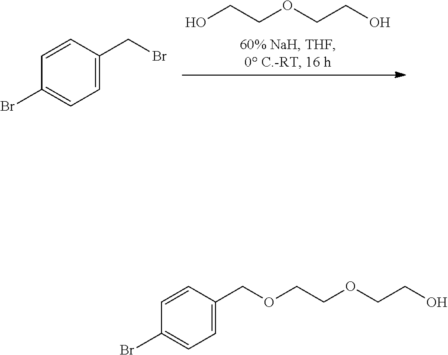 [SMILES: OCCOCCO ; BrCc1ccc(Br)cc1 ; OCCOCCOCc1ccc(Br)cc1]

To a stirred solution of 2,2′-oxybis (ethan-1-ol) (3.9 mL, 40.0 mmol, 5.0 eq) in THF (60 mL) at 0° C., was added 60% NaH (1.92 g, 80.0 mmol, 10 eq) and stirred for 30 min. After that 1-bromo-4-(bromomethyl)benzene (1.92 g, 8.00 mmol, 1 eq) was added to reaction mixture and stirred at room temperature for 16 h. After completion of reaction by TLC, the reaction mixture was diluted with ice cold water (30 mL) and extracted with EtOAc (2×30 mL). The combined organic layer was dried over Na₂SO₄, filtered, and concentrated under reduced pressure to afford crude product. The crude product was purified by silica gel (100-200 mesh) column chromatography [elution with 50% to 70% EtOAc in Hexane] to afford 2-(2-((4-bromobenzyl)oxy)ethoxy)ethan-1-ol as light yellow liquid (2 g, Yield: 91%).
TLC system: EtOAc/Hexane (50:50),
R_{f} value: ˜0.1;

LCMS (m/z): 275.1 (M+H)⁺;

¹H NMR (400 MHz, CDCl₃) δ: 7.47 (d, J=8.0 Hz, 2H), 7.22 (d, J=8.0 Hz, 2H), 4.52 (s, 2H), 3.76-3.68 (m, 4H), 3.65-3.61 (m, 4H).

### Synthesis of 2-(2-((4-(4,4,5,5-tetramethyl-1,3,2-dioxaborolan-2-yl)benzyl)oxy)ethoxy)ethan-1-ol

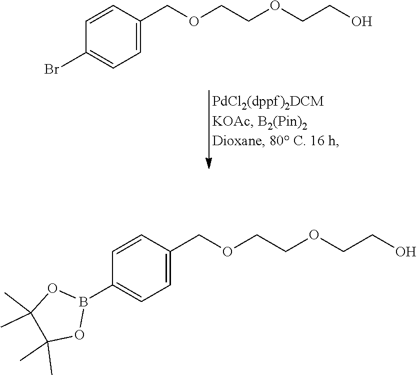 [SMILES: OCCOCCOCc1ccc(Br)cc1 ; CC1(C)OB(c2ccc(COCCOCCO)cc2)OC1(C)C]

In a sealed tube, to a degassed solution of 2-(2-((4-bromobenzyl)oxy)ethoxy)ethan-1-ol (2 g, 7.30 mmol, 1.0 eq) and B₂(pin)₂ (2.2 g, 8.75 mmol, 1.2 eq) in dioxane (20 mL) at room temperature, was added KOAc (1.43 g, 14.6 mmol, 2 eq), Pd(dppf)Cl₂DCM (0.3 g, 0.36 mmol, 0.05 eq) and stirred at 80° C. for 16 h. After completion of reaction by TLC, reaction mixture was filtered through a pad of diatomaceous earth (e.g., Celite®), washed with EtOAc (10 mL), and concentrated under reduced pressure to afford 2-(2-((4-(4,4,5,5-tetramethyl-1,3,2-dioxaborolan-2-yl)benzyl)oxy)ethoxy)ethan-1-ol as brown liquid (2 g, Yield: Crude). Material used as such without any modification.

### Synthesis of 3-(4,6-difluoro-5-(4-((2-(2-hydroxyethoxy)ethoxy)methyl)phenyl)-1H-indol-3-yl)-1,2,4-oxadiazol-5(4H)-one

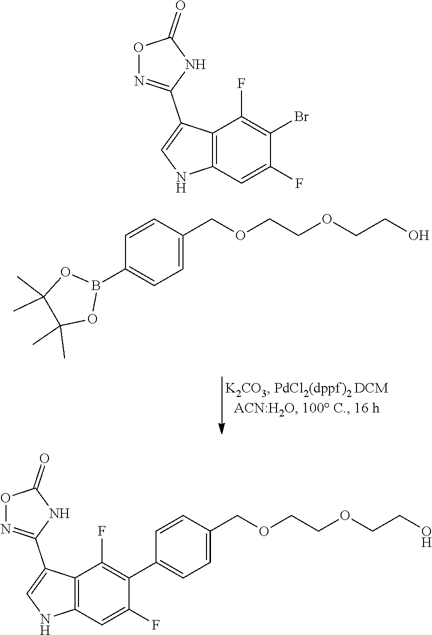

In a sealed tube to a degassed solution of 3-(5-bromo-4,6-difluoro-1H-indol-3-yl)-1,2,4-oxadiazol-5(4H)-one (250 mg, 0.79 mmol, 1.0 eq) and 2-(2-((4-(4,4,5,5-tetramethyl-1,3,2-dioxaborolan-2-yl)benzyl)oxy)ethoxy)ethan-1-ol (715 mg, 2.22 mmol, 2.8 eq) in ACN:H₂O (1:1, 5 mL) at room temperature, was added K₂CO₃ (328 mg, 2.38 mmol, 3 eq) followed by Pd(dppf)Cl₂·DCM (32 mg, 0.04 mmol, 0.05 eq) and stirred at 100° C. for 16 h. After completion of reaction by TLC, the reaction mixture was diluted with water (20 mL) and washed with 10% MeOH/DCM (2×20 mL). Aqueous layer concentrated under reduced pressure and purified by reverse phase column chromatography [elution with 35% ACN in 0.1% FA in H₂O] provided 150 mg semi pure compound. The semi pure product was purified by Prep-HPLC and lyophilized to afford 3-(4,6-difluoro-5-(4-((2-(2-hydroxyethoxy)ethoxy)methyl)phenyl)-1H-indol-3-yl)-1,2,4-oxadiazol-5(4H)-one as an off white solid (68 mg, Yield: 16%).
TLC system: EtOAc (100%),
R_{f} value: ˜0.2;

LCMS (m/z): 430.1 (M−H)⁻;

¹H NMR (400 MHz, DMSO-d₆) δ: 12.28 (s, 1H), 7.99 (s, 1H), 7.47-7.42 (m, 4H), 7.34 (d, J=9.6 Hz, 1H), 4.61 (brs, 1H), 4.57 (s, 2H), 3.62-3.58 (m, 4H), 3.52-3.48 (m, 2H), 3.46-3.43 (m, 2H).

### Prep Conditions:

Column: X-BRIDGE C-18 (250×19) mm 5mic,
Buffer MP-(A): 0.1% FA IN WATER MP-(B): CAN,
Gradient (T/% B): 0/25, 12/50, 15/95.
Flow Rate: 14 mL/min,
Solubility: THF, ACN, WATER.

### SYNTHETIC EXAMPLE 25

### Synthesis of Compound I-46

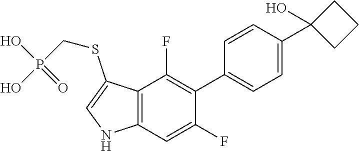 [SMILES: O=P(O)(O)CSc1c[nH]c2cc(F)c(-c3ccc(C4(O)CCC4)cc3)c(F)c12]

### Synthesis of (((5-bromo-4,6-difluoro-1H-indol-3-yl)thio)methyl)phosphonic acid

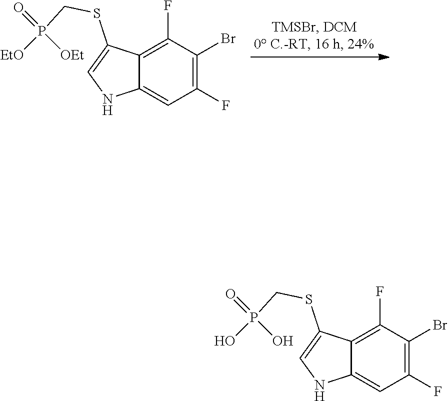 [SMILES: CCOP(=O)(CSc1c[nH]c2cc(F)c(Br)c(F)c12)OCC ; O=P(O)(O)CSc1c[nH]c2cc(F)c(Br)c(F)c12]

To a stirred solution of diethyl (((5-bromo-4,6-difluoro-1H-indol-3-yl)thio)methyl)phosphonate (1.6 g, 3.87 mmol, 1.0 eq) in DCM (16 mL) at 0° C., was added TMSBr (1.97 mL, 15.496 mmol, 4 eq) and stirred at room temperature for 16 h. After completion of reaction by TLC, reaction mixture was concentrated, diluted with water (50 mL) and washed with DCM (2×50 mL). Aqueous layer was concentrated under reduced pressure, purified by reverse phase C₁₈ column chromatography [eluted with 20-25% ACN with 0.1% FA in H₂O] and pure fractions were concentrated to afford (((5-bromo-4,6-difluoro-1H-indol-3-yl)thio)methyl)phosphonic acid as gummy liquid (330 mg, Yield: 24%).
TLC system: MeOH/DCM (10:90),
R_{f} value: ^{˜}0.1,

LCMS (m/z): 358.08 (M+H)⁺.

### Synthesis of (((4,6-difluoro-5-(4-(1-hydroxycyclobutyl)phenyl)-1H-indol-3-yl)thio)methyl)phosphonic acid

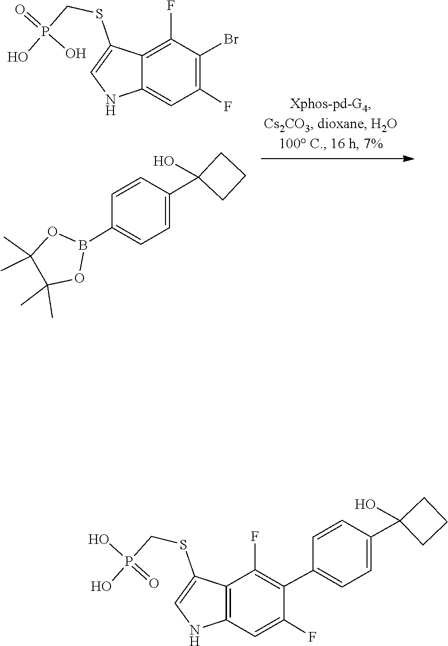

To a stirred solution of (((5-bromo-4,6-difluoro-1H-indol-3-yl)thio)methyl)phosphonic acid (250 mg, 0.70 mmol, 1.0 eq) in Dioxane:H₂O (8:2) (25 mL, 20 Vol), was added 1-(4-(4,4,5,5-tetramethyl-1,3,2-dioxaborolan-2-yl)phenyl)cyclobutan-1-ol (288 mg, 1.05 mmol, 1.5 eq), Cs₂CO₃ (682 mg, 2.10 mmol, 3 eq) and degassed with N₂ for 10 min. Later added Xphos-Pd-G4 (60 mg, 0.070 mmol, 0.1 eq) and stirred at 100° C. for 16 h. After completion of reaction by TLC, the reaction mixture was concentrated and purified by reverse phase C₁₈ column chromatography [eluted with 25-30% ACN with 0.1% ABC in H₂O]. Collected fractions were lyophilized and re-purified by Prep-HPLC, lyophilized to afford (((4,6-difluoro-5-(4-(1-hydroxycyclobutyl)phenyl)-1H-indol-3-yl)thio)methyl)phosphonic acid as off white solid (23 mg, Yield: 7%).
TLC system: MeOH/DCM (10:90),
R_{f} value: ˜0.01;

LCMS (m/z): 424.13 (M−H)⁺.

¹H NMR (400 MHz, DMSO-d₆) δ: 11.88 (s, 1H), 7.58-7.55 (m, 2H), 7.41-7.39 (m, 3H), 7.14 (d, J=10 Hz, 1H), 2.80 (d, J=14 Hz, 2H), 2.49-2.41 (m, 2H), 2.33-2.26 (m, 2H), 1.96-1.91 (m, 1H), 1.71-1.64 (m, 1H).

### Prep-HPLC Method:

Column: YMC C₁₈ (250× 21.2) mm 5mic
Buffer: MP-(A): 10 mM ABC IN WATER MP-(B): Acetonitrile:WATER (80:20)
Gradient: (T/% B) 0/20, 15/55, 25/95
Flow Rate: 13 mL/min
Solubility: WATER, Acetonitrile, THF

### SYNTHETIC EXAMPLE 26

### Synthesis of Compound I-47

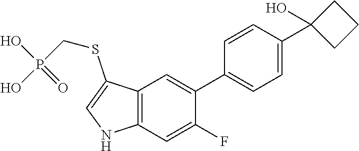 [SMILES: O=P(O)(O)CSc1c[nH]c2cc(F)c(-c3ccc(C4(O)CCC4)cc3)cc12]

### Synthesis of (((5-bromo-6-fluoro-1H-indol-3-yl)thio)methyl)phosphonic acid

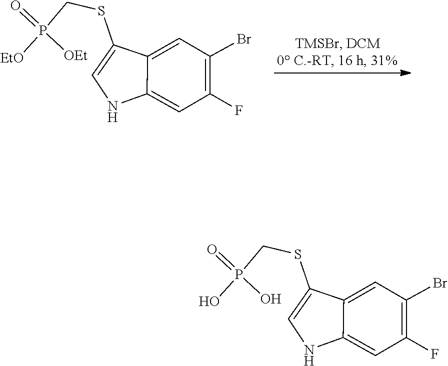 [SMILES: CCOP(=O)(CSc1c[nH]c2cc(F)c(Br)cc12)OCC ; O=P(O)(O)CSc1c[nH]c2cc(F)c(Br)cc12]

To a stirred solution of diethyl (((5-bromo-6-fluoro-1H-indol-3-yl)thio)methyl)phosphonate (1.1 g, 2.78 mmol, 1.0 eq) in DCM (11 mL) at 0° C., was added TMSBr (1.4 mL, 21.22 mmol, 4 eq) and stirred at room temperature for 16 h. After completion of reaction by TLC, volatiles removed, diluted with water (50 mL) and washed with DCM (2×50 mL). Aqueous layer was concentrated under reduced pressure and purified by reverse phase C₁₈ column chromatography [eluted with 20-25% ACN with 0.1% FA in H₂O], pure fractions were concentrated to afford (((5-bromo-6-fluoro-1H-indol-3-yl)thio)methyl)phosphonic acid as gummy liquid (300 mg, Yield: 31%).
TLC system: MeOH/DCM (10:90),
R_{f} value: ^{˜}0.1,

LCMS (m/z): 339.98 (M+H)⁺.

### Synthesis of (((6-fluoro-5-(4-(1-hydroxycyclobutyl)phenyl)-1H-indol-3-yl)thio)methyl)phosphonic acid

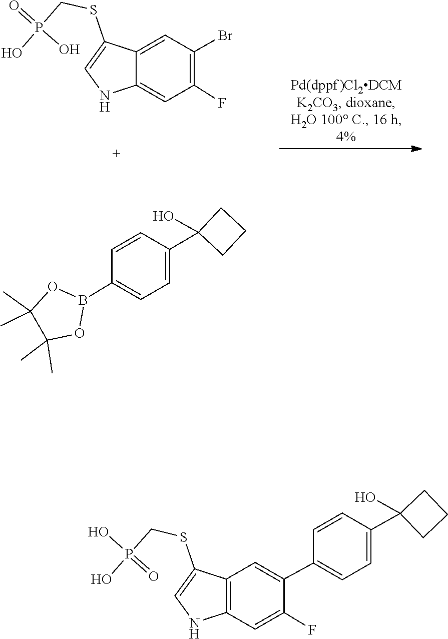 [SMILES: O=P(O)(O)CSc1c[nH]c2cc(F)c(Br)cc12 ; CC1(C)OB(c2ccc(C3(O)CCC3)cc2)OC1(C)C ; O=P(O)(O)CSc1c[nH]c2cc(F)c(-c3ccc(C4(O)CCC4)cc3)cc12]

To a stirred solution of (((5-bromo-6-fluoro-1H-indol-3-yl)thio)methyl)phosphonic acid (250 mg, 0.73 mmol, 1.0 eq) in dioxane: H₂O:ACN (3:1:1) (5 mL, 20 Vol), was added 1-(4-(4,4,5,5-tetramethyl-1,3,2-dioxaborolan-2-yl)phenyl)cyclobutan-1-ol (303 mg, 1.10 mmol, 1.5 eq), K₂CO₃ (305 mg, 2.21 mmol, 3 eq) and degassed with N₂ for 10 min. Later added Pd(dppf)Cl₂·DCM (60 mg, 0.073 mmol, 0.1 eq) and stirred at 100° C. for 16 h. After completion of reaction by TLC, reaction mixture was concentrated, purified by reverse phase C₁₈ column chromatography [eluted with 25-30% ACN with 0.1% ABC in H₂O]. Collected fractions were lyophilized and re-purified by prep-HPLC, lyophilized to afford (((6-fluoro-5-(4-(1-hydroxycyclobutyl)phenyl)-1H-indol-3-yl)thio)methyl)phosphonic acid as off white solid (12 mg, Yield: 4%).
TLC system: MeOH/DCM (10:90),
R_{f} value: ˜0.01;

LCMS (m/z): 406.12 (M+H)⁺.

¹H NMR (400 MHz, DMSO-d₆) δ: 11.39 (s, 1H), 7.65 (d, J=8.0 Hz, 1H), 7.56-7.50 (m, 4H), 7.47 (d, J=1.6 Hz, 1H), 7.27 (d, J=11.6 Hz, 1H), 2.72 (d, J=14.0 Hz, 2H), 2.46-2.39 (m, 2H), 2.33-2.25 (m, 2H), 1.97-1.88 (m, 1H), 1.70-1.63 (m, 1H).

### Prep-HPLC Method:

Column: GEMINI NX C₁₈ (250× 21.2) mm 5mic,
Buffer: MP-(A): 10 Mm ABC IN WATER MP-(B): Acetonitrile:water (80:20),
Gradient: (T/% B) 0/20, 12/49, 15/49, 25/95,
Flow Rate: 14 mL/min,
Solubility: WATER, ACN, THF.

### SYNTHETIC EXAMPLE 27

### Synthesis of Compound I-48

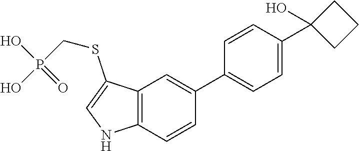 [SMILES: O=P(O)(O)CSc1c[nH]c2ccc(-c3ccc(C4(O)CCC4)cc3)cc12]

### Synthesis of (((5-bromo-1H-indol-3-yl)thio)methyl)phosphonic acid

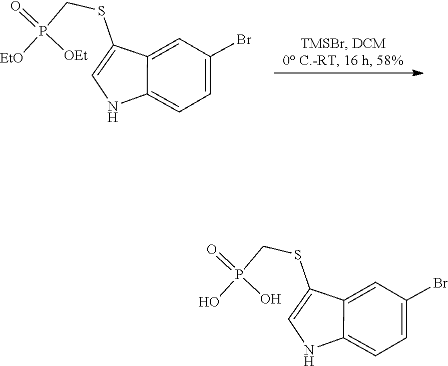 [SMILES: CCOP(=O)(CSc1c[nH]c2ccc(Br)cc12)OCC ; O=P(O)(O)CSc1c[nH]c2ccc(Br)cc12]

To a stirred solution of diethyl (((5-bromo-1H-indol-3-yl)thio)methyl)phosphonate (2 g, 5.30 mmol, 1.0 eq) in DCM (20 mL) at 0° C., was added TMSBr (2.8 mL, 21.2 mmol, 4 eq) and stirred at room temperature for 16 h. After completion of reaction by TLC, volatiles removed under vacuum, diluted with water (50 mL) and washed with DCM (2×50 mL). Aqueous layer was concentrated under reduced pressure and purified by reverse phase C₁₈ column chromatography [eluted with 30% ACN with 0.1% FA in H₂O], pure fractions were concentrated to afford (((5-bromo-1H-indol-3-yl)thio)methyl)phosphonic acid as gummy solid (1 g, Yield: 58%).
TLC system: MeOH/DCM (10:90),
R_{f} value: ^{˜}0.1,

LCMS (m/z): 322.0 (M+H)⁺.

### Synthesis of (((5-(4-(1-hydroxycyclobutyl)phenyl)-1H-indol-3-yl)thio)methyl)phosphonic acid

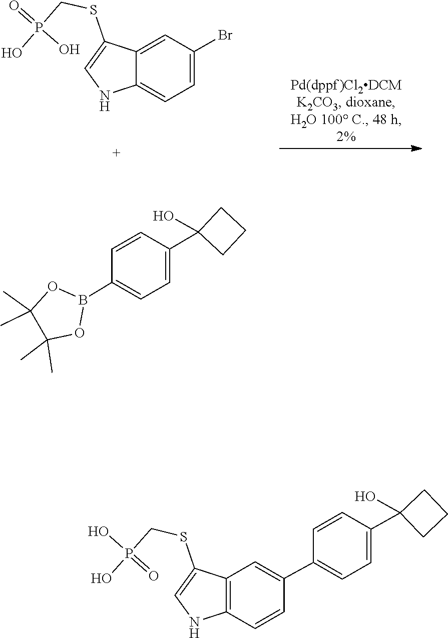 [SMILES: O=P(O)(O)CSc1c[nH]c2ccc(Br)cc12 ; CC1(C)OB(c2ccc(C3(O)CCC3)cc2)OC1(C)C ; O=P(O)(O)CSc1c[nH]c2ccc(-c3ccc(C4(O)CCC4)cc3)cc12]

To a stirred solution of (((5-bromo-1H-indol-3-yl)thio)methyl)phosphonic acid (500 mg, 1.55 mmol, 1.0 eq) in Dioxane:H₂O:ACN (3:1:1) (10 mL, 20 Vol), was added 1-(4-(4,4,5,5-tetramethyl-1,3,2-dioxaborolan-2-yl)phenyl)cyclobutan-1-ol (640 mg, 2.33 mmol, 1.5 eq), K₂CO₃ (645 mg, 4.67 mmol, 3 eq) and degassed with N₂ for 10 min. Later, added Pd(dppf)Cl₂·DCM (127 mg, 0.16 mmol, 0.1 eq) and stirred at 100° C. for 48 h. After completion of reaction by TLC, reaction mixture was concentrated and purified by reverse phase C₁₈ column chromatography [eluted with 25-30% ACN with 0.1% ABC in H₂O]. Collected fractions were lyophilized, re-purified by Prep-HPLC and lyophilized to afford (((5-(4-(1-hydroxycyclobutyl)phenyl)-1H-indol-3-yl)thio)methyl)phosphonic acid as off white solid (12 mg, Yield: 2%).
TLC system: MeOH/DCM (10:90),
R_{f} value: ˜0.01;

LCMS (m/z): 388.5 (M+H)⁺.

¹H NMR (400 MHz, DMSO-d₆) δ: 11.34 (s, 1H), 7.88 (s, 1H), 7.64 (d, J=8.4 Hz, 2H), 7.53 (d, J=8.4 Hz, 2H), 7.49 (d, J=2.4 Hz, 1H), 7.44-7.40 (m, 2H), 2.76 (d, J=14.0 Hz, 2H), 2.44-2.38 (m, 2H), 2.33-2.24 (m, 2H), 1.96-1.86 (m, 1H), 1.70-1.61 (m, 1H)

### Prep-HPLC Method:

Column: GEMINI C₁₈ (250×21.2) mm 5mic

Buffer: MP-(A): 10 Mm abc IN WATER MP-(B): Acetonotrile:WATER (80:20)
Gradient: (T/% B) 0/30, 15/55, 25/95
Flow Rate: 12 mL/min
Solubility: water, THF, acetonitrile

### SYNTHETIC EXAMPLE 28

### Synthesis of Compound I-49

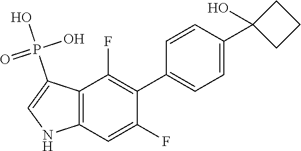 [SMILES: O=P(O)(O)c1c[nH]c2cc(F)c(-c3ccc(C4(O)CCC4)cc3)c(F)c12]

### Synthesis of (5-bromo-4,6-difluoro-1H-indol-3-yl)phosphonic acid

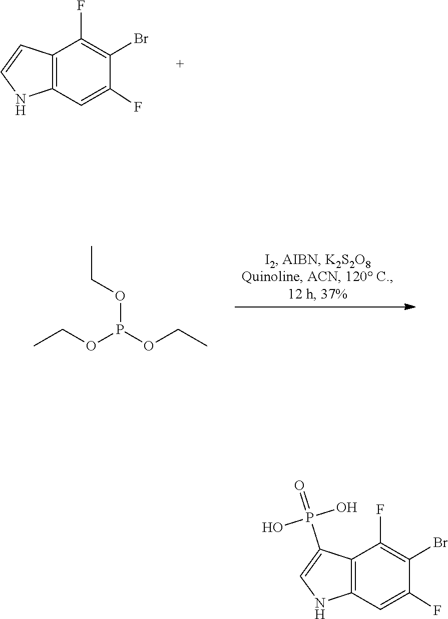

To a stirred solution of 5-bromo-4,6-difluoro-1H-indole (500 mg, 2.16 mmol, 1.0 eq) and triethyl phosphite (1.1 mL, 6.49 mmol, 3 eq) in ACN (10 mL) at room temperature, was added K₂S₂O₈ (1.1 g, 4.32 mmol, 2.0 eq), AIBN (533 mg, 3.25 mmol, 1.5 eq), Quinoline (0.12 mL, 1.08 mmol, 0.05 eq) and stirred at 120° C. for 12 h. After completion of reaction by TLC, the reaction mixture was concentrated under reduced pressure and purified by reverse phase column chromatography [elution with 16-18% ACN in 0.1% FA in H₂O] to afford (5-bromo-4,6-difluoro-1H-indol-3-yl)phosphonic acid as off white solid (250 mg, Yield: 37%).
TLC system: MeOH/DCM (10:90),
R_{f} value: ˜0.2;

LCMS (m/z): 309.95 [M−H]⁻;

### Synthesis of (4,6-difluoro-5-(4-(1-hydroxycyclobutyl)phenyl)-1H-indol-3-yl)phosphonic acid as bicarbonate salt

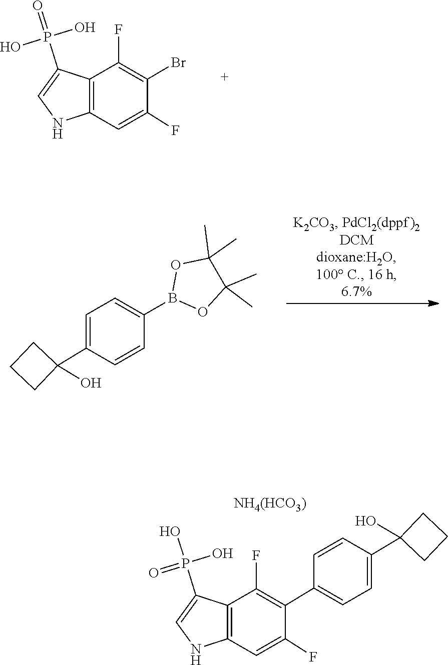 [SMILES: O=P(O)(O)c1c[nH]c2cc(F)c(Br)c(F)c12 ; CC1(C)OB(c2ccc(C3(O)CCC3)cc2)OC1(C)C ; O=P(O)(O)c1c[nH]c2cc(F)c(-c3ccc(C4(O)CCC4)cc3)c(F)c12 ; "NH4(HCO3)"]

In a sealed tube, to a degassed solution of (5-bromo-4,6-difluoro-1H-indol-3-yl)phosphonic acid (450 mg, 1.456 mmol, 1.0 eq) and 1-(4-(4,4,5,5-tetramethyl-1,3,2-dioxaborolan-2-yl)phenyl)cyclobutan-1-ol (997 mg, 3.64 mmol, 2.5 eq) in Dioxane:H₂O (2:1, 15 mL) at room temperature, was added K₂CO₃ (602 mg, 4.37 mmol, 2.0 eq) followed by Pd(dppf)Cl₂·DCM (118 mg, 0.145 mmol, 0.1 eq) and stirred at 100° C. for 16 h. After completion of reaction by TLC, the reaction mixture was concentrated under reduced pressure and purified by reverse phase column chromatography [elution with 35-40% ACN in 0.1% FA in H₂O] provided semi-pure material. Re-purified by Prep-HPLC and lyophilized to afford (4,6-difluoro-5-(4-(1-hydroxycyclobutyl)phenyl)-1H-indol-3-yl)phosphonic acid as bicarbonate salt as off white solid (37 mg, Yield: ˜6.7%).
TLC system: MeOH/DCM (10:90),
R_{f} value: ˜0.1;

LCMS (m/z): 378.11 [M−H]⁻,

¹H NMR (400 MHz, DMSO-d₆) δ: 11.32 (s, 1H), 7.59-7.57 (m, 2H), 7.39-7.37 (m, 3H), 7.12-7.08 (m, 4H), 5.55 (s, 1H), 2.47-2.41 (m, 2H), 2.33-2.26 (m, 2H), 1.98-1.91 (m, 1H), 1.73-1.66 (m, 1H).
Column name: Gemini NX C₁₈ (250×21.2) mm 5 micron
Mobile phase (A): 10 mm ABC in water, Mobile phase (B): ACN
Loading: 15 mg/injection
Method (% B): 0/20,15/43,20/95,25/95
Flow Rate: 13 mL/min
Solubility: acetonitrile, water

### SYNTHETIC EXAMPLE 29

### Synthesis of Compound I-55

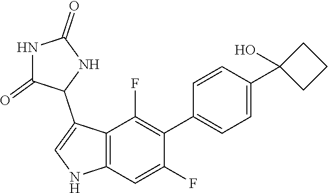 [SMILES: O=C1NC(=O)C(c2c[nH]c3cc(F)c(-c4ccc(C5(O)CCC5)cc4)c(F)c23)N1]

### Synthesis of 5-(5-bromo-4,6-difluoro-1H-indol-3-yl)imidazolidine-2,4-dione

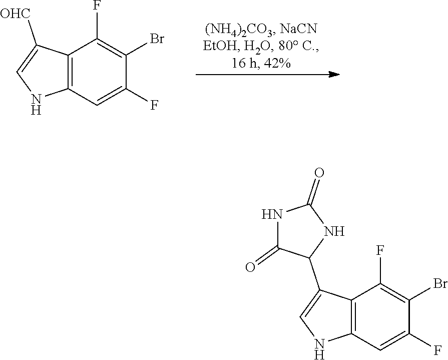 [SMILES: O=Cc1c[nH]c2cc(F)c(Br)c(F)c12 ; O=C1NC(=O)C(c2c[nH]c3cc(F)c(Br)c(F)c23)N1]

To a stirred solution of 5-bromo-4,6-difluoro-1H-indole-3-carbaldehyde (1.5 g, 4.17 mmol, 1.0 eq) and (NH₄)₂CO₃ (1.2 g, 12.5 mmol, 3 eq) in EtOH:H₂O (1:1, 21 mL) at room temperature, was added NaCN (300 mg, 6.20 mmol, 1.5 eq) and stirred at 80° C. for 16 h. After completion of reaction by TLC, reaction mixture was concentrated under reduced pressure and purified by silica gel (60-120 mesh) column chromatography [elution with 100% EtOAc] to afford 5-(5-bromo-4,6-difluoro-1H-indol-3-yl)imidazolidine-2,4-dione as brown solid (800 mg, Yield: 42%).
TLC system: EtOAc (100%),
R_{f} value: ˜0.2;

LCMS (m/z): 327.9 [M−H]⁻;

### Synthesis of 5-(4,6-difluoro-5-(4-(1-hydroxycyclobutyl)phenyl)-1H-indol-3-yl)imidazolidine-2,4-dione

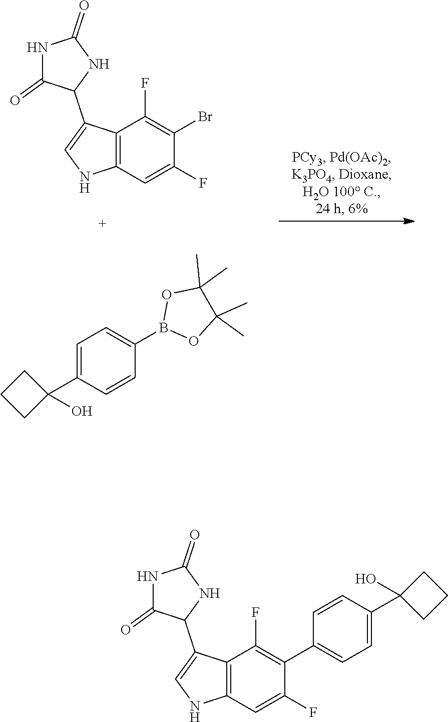 [SMILES: O=C1NC(=O)C(c2c[nH]c3cc(F)c(Br)c(F)c23)N1 ; CC1(C)OB(c2ccc(C3(O)CCC3)cc2)OC1(C)C ; O=C1NC(=O)C(c2c[nH]c3cc(F)c(-c4ccc(C5(O)CCC5)cc4)c(F)c23)N1]

In a sealed tube, to a degassed solution of 5-(5-bromo-4,6-difluoro-1H-indol-3-yl)imidazolidine-2,4-dione (800 mg, 2.43 mmol, 1.0 eq) and 1-(4-(4,4,5,5-tetramethyl-1,3,2-dioxaborolan-2-yl)phenyl)cyclobutan-1-ol (668 mg, 2.43 mmol, 1 eq) in Dioxane:H₂O (8:1, 20 mL) at room temperature, was added K₃PO₄ (1.5 g, 7.31 mmol, 3.0 eq), PCy₃ (136 mg, 0.48 mmol, 0.2 eq), followed by Pd(OAc)₂ (54 mg, 0.24 mmol, 0.1 eq) and stirred at 100° C. for 24 h. After completion of reaction by TLC, the reaction mixture was diluted with ice cold water (50 mL) and extracted with EtOAc (2×70 mL). The combined organic layer was dried over Na₂SO₄, filtered and concentrated under reduced pressure to afford crude product. The crude product was purified by silica gel (60-120 mesh) column chromatography [elution with 100% EtOAc] followed by Prep-HPLC purification and lyophilization afforded. 5-(4,6-difluoro-5-(4-(1-hydroxycyclobutyl)phenyl)-1H-indol-3-yl)imidazolidine-2,4-dione as off white solid (60 mg, Yield: ˜6%).
TLC system: EtOAc (100),
R_{f} value: ˜0.2;

LCMS (m/z): 396.1 [M−H]⁺;

¹H NMR (400 MHz, DMSO-d₆) δ: 11.55 (s, 1H), 10.64 (s, 1H), 8.15 (s, 1H), 7.59-7.57 (m, 2H), 7.51 (d, J=2.4 Hz, 1H), 7.35 (d, J=8 Hz, 2H), 7.20 (d, J=10 Hz, 1H), 5.52 (br s, 1H), 5.37 (s, 1H), 2.49-2.41 (m, 2H), 2.34-2.26 (m, 2H), 1.97-1.91 (m, 1H), 1.71-1.65 (m, 1H).
Column: GEMINI C₁₈ (250× 21.2) mm 5mic
Buffer: MP-(A): 0.1% TFA IN WATER MP-(B): ACN
Gradient: (T/% B) 0/25, 15/50, 25/95
Flow Rate: 13 mL/min
Solubility: water, THF, acetonitrile

### SYNTHETIC EXAMPLE 30

### Synthesis of Compound I-56

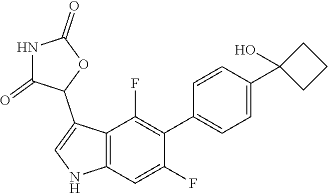 [SMILES: O=C1NC(=O)C(c2c[nH]c3cc(F)c(-c4ccc(C5(O)CCC5)cc4)c(F)c23)O1]

### Synthesis of 1-(4-(4,6-difluoro-1H-indol-5-yl)phenyl)cyclobutan-1-ol

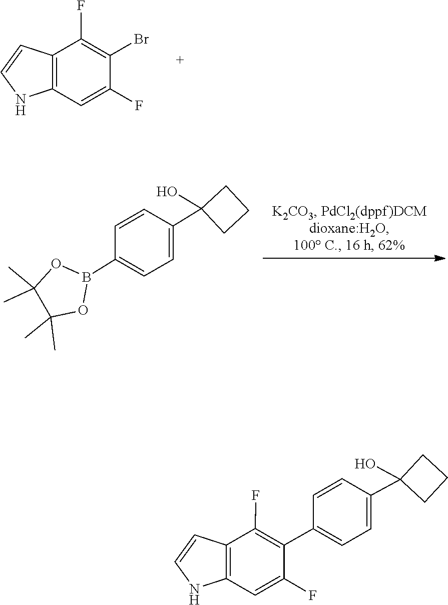 [SMILES: Fc1cc2[nH]ccc2c(F)c1Br ; CC1(C)OB(c2ccc(C3(O)CCC3)cc2)OC1(C)C ; OC1(c2ccc(-c3c(F)cc4[nH]ccc4c3F)cc2)CCC1]

To a stirred solution of 5-bromo-4,6-difluoro-1H-indole (1 g, 4.32 mmol, 1 eq) in dioxane and water (2:1) (10 mL), was added K₂CO₃ (1.8 g, 12.9 mmol, 3 eq), 1-(4-(4,4,5,5-tetramethyl-1,3,2-dioxaborolan-2-yl)phenyl)cyclobutan-1-ol and degassed for 10 min later added Pd(dppf)Cl₂DCM (350 mg, 0.43 mmol, 0.1 eq). RM degassed for 10 more min and the reaction mixture was stirred at 100° C. for 16 h. After completion of reaction by TLC, reaction mixture filtered through a pad of diatomaceous earth (e.g., Celite®) and washed with ethyl acetate. Filtrate was washed with brine solution, dried over sodium sulfate and concentrated to afford crude compound. Purified by silica (100-200 mesh) column chromatography [eluted with 30% EtOAc/Hexane] and pure fractions were concentrated under reduced pressure to afford 1-(4-(4,6-difluoro-1H-indol-5-yl)phenyl)cyclobutan-1-ol as gummy liquid (800 mg, yield: 62%).
TLC system: EtOAC/Hexane (30:70)
R_{f} value: ˜0.3;

LCMS (m/z): 298.15 (M−H)⁺;

¹H NMR (400 MHz, CDCl₃) δ 8.31 (s, 1H), 7.62-7.59 (m, 2H), 7.54-7.52 (m, 2H), 7.20-7.18 (m, 1H), 7.04-7.02 (m, 1H), 6.66-6.64 (m, 1H), 2.67-2.61 (m, 2H), 2.45-2.38 (m, 2H), 2.07-2.04 (m, 1H), 1.79-1.70 (m, 1H).

### Synthesis of 5-(4,6-difluoro-5-(4-(1-hydroxycyclobutyl)phenyl)-1H-indol-3-yl)-5-hydroxypyrimidine-2,4,6 (1H,3H,5H)-trione

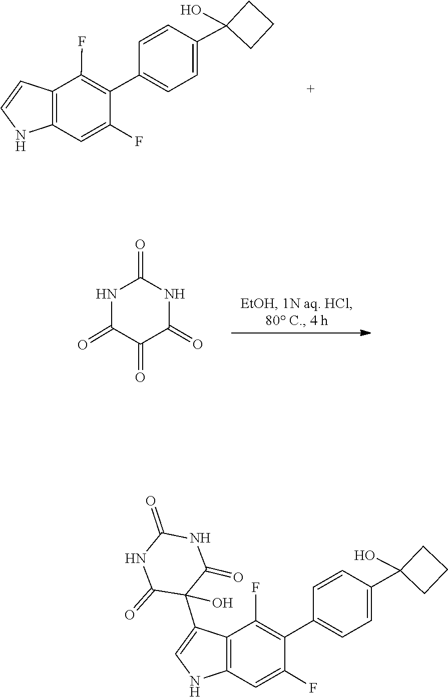 [SMILES: OC1(c2ccc(-c3c(F)cc4[nH]ccc4c3F)cc2)CCC1 ; O=C1NC(=O)C(=O)C(=O)N1 ; O=C1NC(=O)C(O)(c2c[nH]c3cc(F)c(-c4ccc(C5(O)CCC5)cc4)c(F)c23)C(=O)N1]

To a stirred and solution of 1-(4-(4,6-difluoro-1H-indol-5-yl)phenyl)cyclobutan-1-ol (800 mg, 2.67 mmol, 1.0 eq) in EtOH (8 mL, 10 Vol) at room temperature was added Alloxan (432 mg, 2.67 mmol, 1.0 eq) and 1N aq. HCl (6.4 mL, 8 Vol) drop-wise and reaction mixture was stirred at 80° C., for 4 h. After completion of reaction by TLC, reaction mixture cooled to room temperature, evaporated under vacuum, washed with water provided 5-(4,6-difluoro-5-(4-(1-hydroxycyclobutyl)phenyl)-1H-indol-3-yl)-5-hydroxypyrimidine-2,4,6 (1H,3H,5H)-trione as white color solid (800 mg, crude).
TLC system: EtOAc/Hexane (50:50),
R_{f} value: ˜0.1;

LCMS (m/z): 440.28 (M−H)⁺.

### Synthesis of 5-(4,6-difluoro-5-(4-(1-hydroxycyclobutyl)phenyl)-1H-indol-3-yl)oxazolidine-2,4-dione

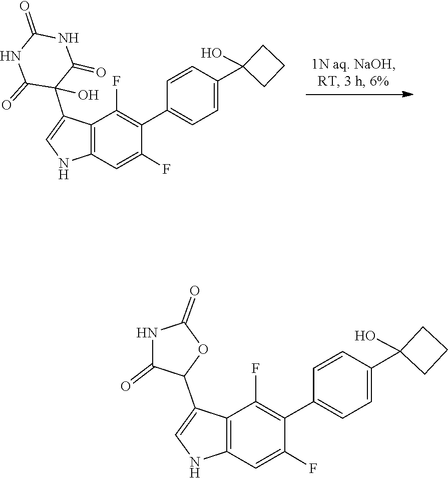 [SMILES: O=C1NC(=O)C(O)(c2c[nH]c3cc(F)c(-c4ccc(C5(O)CCC5)cc4)c(F)c23)C(=O)N1 ; O=C1NC(=O)C(c2c[nH]c3cc(F)c(-c4ccc(C5(O)CCC5)cc4)c(F)c23)O1]

To a stirred solution of 5-(4,6-difluoro-5-(4-(1-hydroxycyclobutyl)phenyl)-1H-indol-3-yl)-5-hydroxypyrimidine-2,4,6 (1H,3H,5H)-trione (800 mg, 1.8 mmol, 1.0 eq) at room temperature, was added 1N aq. NaOH (12 mL) and stirred at RT for 3 h. After completion of reaction by TLC, reaction mixture was acidified with conc HCl (8 mL) and extracted with ethyl acetate (2×100 mL). Organic layer was washed with aq. NaHCO₃ solution, dried over sodium sulfate, and concentrated under reduced pressure. The crude material was purified by reverse phase column chromatography (Grace) [elution with 30-35% of ACN in 0.1% FA in H₂O], followed by Prep-HPLC and pure fraction were lyophilized to afford 5-(4,6-difluoro-5-(4-(1-hydroxycyclobutyl)phenyl)-1H-indol-3-yl)oxazolidine-2,4-dione as white solid (62 mg, yield: 6%).
TLC system: EtOAc,
R_{f} value: ˜0.2;

LCMS: 397.4 [M−H]⁻;

¹H NMR (400 MHz, DMSO-d₆) § 11.9 (s, 1H), 11.85 (s, 1H), 7.76 (d, J=2.8 Hz, 1H), 7.59 (dd, J=4.8 & 1.6 Hz, 2H), 7.36 (d, J=8.4 Hz, 2H), 7.27 (d, J=10.0 Hz, 1H), 6.27 (s, 1H), 5.54 (s, 1H), 2.48-2.41 (m, 2H), 2.34-2.26 (m, 2H), 1.97-1.90 (m, 1H), 1.72-1.65 (m, 1H).
Column: GEMINI NX C₁₈ (250×21.2) mm 5mic
Buffer: MP-(A): 0.1% FA IN WATER MP-(B): Acetonitrile:water (80:20)
Gradient: (T/% B) 0/35, 15/60, 25/95
Flow Rate: 12 mL/min
Solubility: Water, Acetonitrile, THF

### SYNTHETIC EXAMPLE 31

### Synthesis of Compound I-5

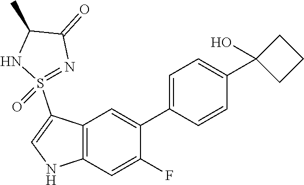 [SMILES: C[C@@H]1NS(=O)(c2c[nH]c3cc(F)c(-c4ccc(C5(O)CCC5)cc4)cc23)=NC1=O]

### Synthesis of 5-bromo-6-fluoro-1H-indole-3-sulfonyl chloride

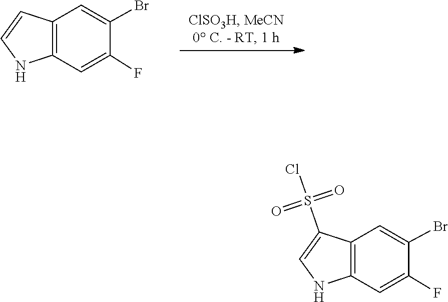 [SMILES: Fc1cc2[nH]ccc2cc1Br ; O=S(=O)(Cl)c1c[nH]c2cc(F)c(Br)cc12]

To a stirred solution of 5-bromo-6-fluoro-1H-indole (5 g, 23.47 mmol, 1.0 eq) in MeCN (125 mL) at 0° C., was added ClSO₃H (1.8 mL, 28.16 mmol, 1.2 eq) and reaction mixture was stirred at room temperature for 1 h. After completion of reaction by TLC, the reaction mixture was concentrated, diluted with water (30 mL) and extracted with EtOAc (2×70 mL). The combined organic layer was dried over Na₂SO₄, filtered and concentrated under reduced pressure to afford crude product. The crude product was triturated with n-Pentane (30 mL), decanted and dried under reduced pressure to afford 5-bromo-6-fluoro-1H-indole-3-sulfonyl chloride as a pale-yellow solid (5 g, Crude). TLC system: EtOAc/Hexane (20:80), R_{f} value: ˜0.4; LCMS (m/z): 292.10 sulfonic acid m/z

### Synthesis of 5-bromo-6-fluoro-1H-indole-3-sulfonamide

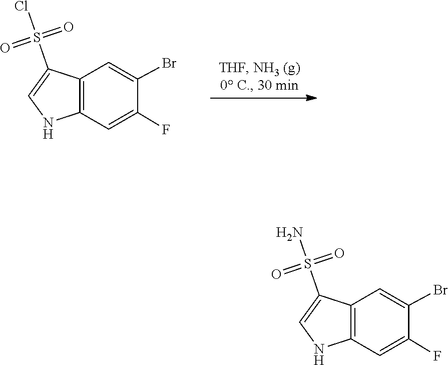 [SMILES: O=S(=O)(Cl)c1c[nH]c2cc(F)c(Br)cc12 ; NS(=O)(=O)c1c[nH]c2cc(F)c(Br)cc12]

To a stirred solution of 5-bromo-6-fluoro-1H-indole-3-sulfonyl chloride (5 g, 16.08 mmol, 1.0 eq) (Crude) in THF (100 mL) at 0° C., NH₃ gas was purged to the reaction mixture for 30 min. After completion of reaction by TLC, the reaction mixture was diluted with water (70 mL) and extracted with EtOAc (2×70 mL). The combined organic layer was dried over Na₂SO₄, filtered and concentrated under reduced pressure to afford crude product. The crude product was triturated with n-pentane (50 mL), decanted and dried under reduced pressure to afford 5-bromo-6-fluoro-1H-indole-3-sulfonamide as an off white solid (5.2 g, Yield: Crude). TLC system: EtOAc/Hexane (50:50), R_{f} value: ˜0.3;

LCMS (m/z): 292.9 (M−H)

### Synthesis of 5-bromo-N-(tert-butyldimethylsilyl)-6-fluoro-1H-indole-3-sulfonamide

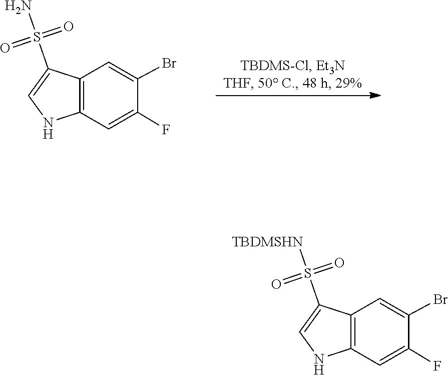

To a stirred solution of 5-bromo-6-fluoro-1H-indole-3-sulfonamide (5.2 g, 17.80 mmol, 1.0 eq) (Crude) in THF (52 mL) at room temperature, was added Et₃N (12.5 mL, 89.00 mmol, 5.0 eq) and TBDMS-Cl (10.68 g, 71.20 mmol, 4.0 eq). The reaction mixture was stirred at 50° C. for 48 h. After completion of reaction by TLC, the reaction mixture was diluted with ice cold water (60 mL) and extracted with EtOAc (2×80 mL). The combined organic layer was dried over Na₂SO₄, filtered and concentrated under reduced pressure to afford crude product. The crude product was purified by silica gel (100-200) column chromatography [elution with 20-25% EtOAc in Hexane] to afford 5-bromo-N-(tert-butyldimethylsilyl)-6-fluoro-1H-indole-3-sulfonamide as a pale-yellow solid (2.8 g, 3-Steps Yield: 29%). TLC system: EtOAc/Hexane (50:50), R_{f} value: ˜0.5; ¹H NMR (400 MHz, CDCl₃) δ: 8.59 (s, 1H), 8.12-8.07 (m, 1H), 7.72-7.70 (m, 1H), 7.23-7.21 (m, 1H), 4.36 (s, 1H), 0.89 (s, 9H), 0.22 (s, 6H).

### Synthesis of tert-butyl 5-bromo-3-(N-(tert-butyldimethylsilyl)sulfamoyl)-6-fluoro-1H-indole-1-carboxylate

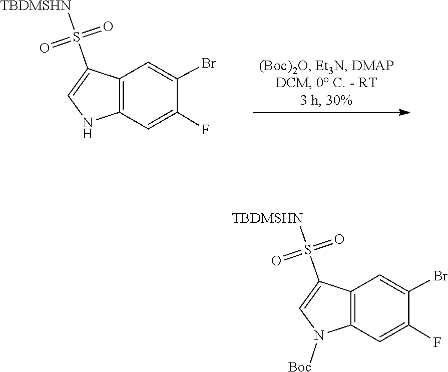

To a stirred solution of 5-bromo-N-(tert-butyldimethylsilyl)-6-fluoro-1H-indole-3-sulfonamide (2.8 g, 6.90 mmol, 1.0 eq) in DCM (30 mL) at 0° C., was added Et₃N (2.42 mL, 17.25 mmol, 2.5 eq), (Boc)₂O (3.15 mL, 13.80 mmol, 2.0 eq) followed by DMAP (253 mg, 2.07 mmol, 0.3 eq). The reaction mixture was stirred at room temperature for 3 h. After completion of reaction by TLC, the reaction mixture was diluted with ice cold water (40 mL) and extracted with DCM (2× 60 mL). The combined organic layer was dried over Na₂SO₄, filtered and concentrated under reduced pressure to afford crude product. The crude product was purified by silica gel (100-200) column chromatography [elution with 10-15% EtOAc in Hexane] to afford tert-butyl 5-bromo-3-(N-(tert-butyldimethylsilyl)sulfamoyl)-6-fluoro-1H-indole-1-carboxylate as off white solid (1.06 g, Yield: 30%). TLC system: EtOAc/Hexane (20:80), R_{f} value: ˜0.6; ¹H NMR (400 MHz, CDCl₃) δ: 8.07 (d, J=6.4 Hz, 1H), 8.03 (s, 1H), 8.00 (d, J=9.2 Hz, 1H), 4.49 (s, 1H), 1.69 (s, 9H), 0.92 (s, 9H), 0.26 (s, 6H).

### Synthesis of tert-butyl(S)-5-bromo-3-(N′-(tert-butyldimethylsilyl)-N-(1-methoxy-1-oxopropan-2-yl)sulfamidimidoyl)-6-fluoro-1H-indole-1-carboxylate

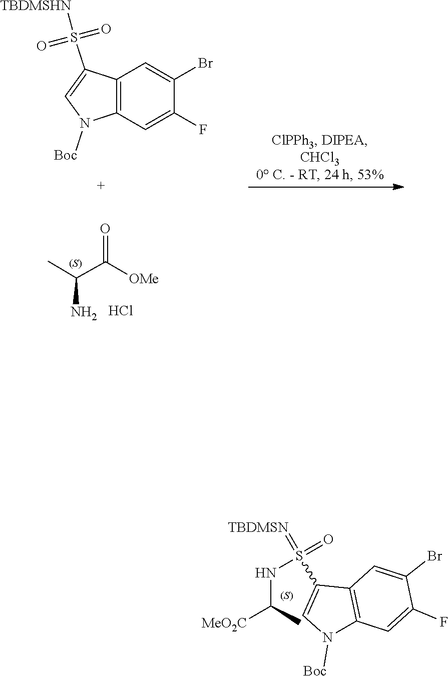

To a stirred solution of PPh₃Cl₂ (787 mg, 2.37 mmol, 1.5 eq) in CHCl₃ (20 mL) at 0° C., was added DIPEA (1.3 mL, 7.90 mmol, 5.0 eq) and stirred for 1 h. Later, a solution of tert-butyl 5-bromo-3-(N-(tert-butyldimethylsilyl)sulfamoyl)-6-fluoro-1H-indole-1-carboxylate (800 mg, 1.58 mmol, 1.0 eq) in CHCl₃ (10 mL) was added to reaction mixture and continued for 1 h. At same temperature, added solution of L-Alanine methyl ester HCl (663 mg, 4.74 mmol, 3.0 eq) in CHCl₃ (10 mL) and stirred for 24 h at room temperature. After completion of reaction by TLC, the reaction mixture was quenched with ice cold water (40 mL) and extracted with DCM (2×40 mL). The combined organic layer was dried over Na₂SO₄, filtered and concentrated under reduced pressure to afford crude product. The crude product was purified by silica gel (100-200) column chromatography [elution with 10% EtOAc in Hexane] to afford tert-butyl(S)-5-bromo-3-(N′-(tert-butyldimethylsilyl)-N-(1-methoxy-1-oxopropan-2-yl)sulfamidimidoyl)-6-fluoro-1H-indole-1-carboxylate as a gummy liquid (500 mg, Yield: 53%). TLC system: EtOAc/Hexane (10:90), R_{f} value: ˜0.4; LCMS (m/z): 592.2 (M+H)⁺.

### Synthesis of(S)-1-(5-bromo-6-fluoro-1H-indol-3-yl)-4-methyl-4,5-dihydro-3H-1λ⁶,2,5-thiadiazol-3-one 1-oxide

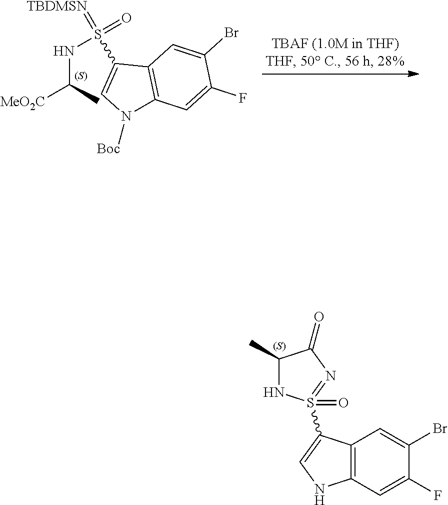

To a stirred solution of tert-butyl(S)-5-bromo-3-(N′-(tert-butyldimethylsilyl)-N-(1-methoxy-1-oxopropan-2-yl)sulfamidimidoyl)-6-fluoro-1H-indole-1-carboxylate (600 mg, 1.01 mmol, 1.0 eq) in THF (5 mL) at 0° C., was added TBAF (1.0 M in THF) (5 mL, 10 Vol). The reaction mixture was stirred at 50° C. for 56 h. After completion of reaction by TLC, the reaction mixture was quenched with NH₄Cl solution (20 mL) and extracted with DCM (2×50 mL). The combined organic layer was dried over Na₂SO₄, filtered and concentrated under reduced pressure to afford crude product. The crude product was purified by reverse phase column chromatography [elution 60% ACN in 0.1% FA in H₂O] to afford (S)-1-(5-bromo-6-fluoro-1H-indol-3-yl)-4-methyl-4,5-dihydro-3H-1λ⁶,2,5-thiadiazol-3-one 1-oxide as brown gummy solid (100 mg, Yield: 28%). TLC system: MeOH/DCM (10:90), R_{f} value: ˜0.4; LCMS (m/z): 346.0 (M+H)⁺. Diastereomeric mixture

### Synthesis of(S)-1-(6-chloro-5-(4-(1-hydroxycyclobutyl)phenyl)-1H-indol-3-yl)-4-methyl-4,5-dihydro-3H-1λ⁶,2,5-thiadiazol-3-one 1-oxide

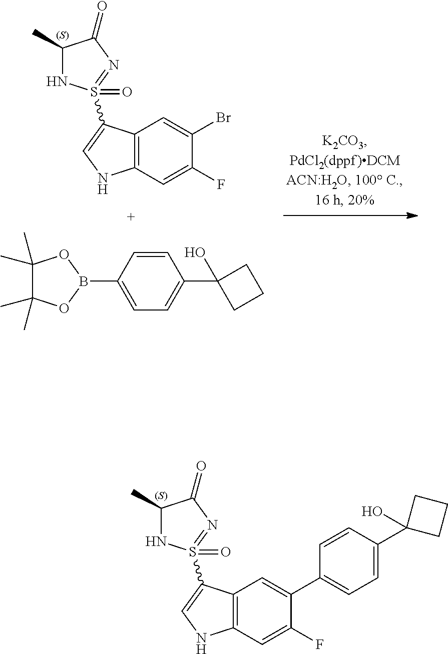

In a sealed tube to a degassed solution of(S)-1-(5-bromo-6-fluoro-1H-indol-3-yl)-4-methyl-4,5-dihydro-3H-1λ⁶,2,5-thiadiazol-3-one 1-oxide (100 mg, 0.28 mmol, 1.0 eq) and 1-(4-(4,4,5,5-tetramethyl-1,3,2-dioxaborolan-2-yl)phenyl)cyclobutan-1-ol (127 mg, 0.45 mmol, 1.6 eq) in ACN:H₂O (1:1, 2 mL) at room temperature, was added K₂CO₃ (77 mg, 0.56 mmol, 2.0 eq) followed by PdCl₂(dppf)·DCM (23 mg, 0.028 mmol, 0.1 eq) and stirred at 100° C. for 16 h. After completion of reaction by TLC, the reaction mixture was diluted with water (15 mL) and extracted with 10% MeOH in DCM (2×15 mL). The combined organic layer was dried over Na₂SO₄, filtered and concentrated under reduced pressure to afford crude product. Purified by Prep-HPLC and lyophilized to afford (S)-1-(6-chloro-5-(4-(1-hydroxycyclobutyl)phenyl)-1H-indol-3-yl)-4-methyl-4,5-dihydro-3H-1λ⁶,2,5-thiadiazol-3-one 1-oxide as off white solid (23 mg, Yield: 20%). TLC system: MeOH/DCM (10:90), R_{f} value: ˜0.2; LCMS (m/z): 414.32 (M+H)⁺; ¹H NMR (400 MHz, DMSO-d₆) δ: 12.50 (br, 1H), 9.18-9.09 (m, 1H), 8.26-8.24 (m, 1H), 7.61-7.59 (m, 2H), 7.52-7.43 (m, 3H), 7.40 (d, J=7.2 Hz, 1H), 5.56 (s, 1H), 4.30-4.27 (m, 1H), 2.45-2.42 (m, 2H), 2.33-2.30 (m, 2H), 1.95-1.92 (m, 1H), 1.70-1.66 (m, 1H), 1.39 (dd, J=6.8 Hz, 13.6 Hz, 3H). (Diastereomeric mixture).

### SYNTHETIC EXAMPLE 32

### Synthesis of Compound I-20

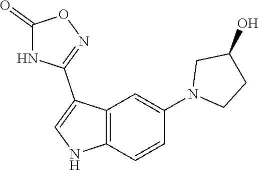 [SMILES: O=c1[nH]c(-c2c[nH]c3ccc(N4CC[C@H](O)C4)cc23)no1]

### Synthesis of(S)-5-(3-((tert-butyldimethylsilyl)oxy)pyrrolidin-1-yl)-1-((2-(trimethylsilyl)ethoxy)methyl)-1H-indole-3-carbonitrile

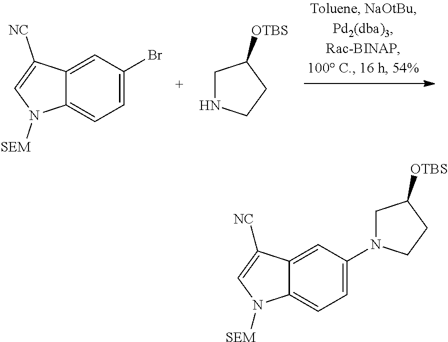 [SMILES: C[Si](C)(C)CCOCn1cc(C#N)c2cc(Br)ccc21 ; CC(C)(C)[Si](C)(C)O[C@H]1CCNC1 ; CC(C)(C)[Si](C)(C)O[C@H]1CCN(c2ccc3c(c2)c(C#N)cn3COCC[Si](C)(C)C)C1]

To a stirred solution of 5-bromo-1-((2-(trimethylsilyl)ethoxy)methyl)-1H-indole-3-carbonitrile (1.5 g, 4.27 mmol, 1.0 eq) in Toluene (45 mL) at room temperature, was added (S)-3-((tert-butyldimethylsilyl)oxy)pyrrolidine (1.03 g, 5.12 mmol, 1.2 eq) and NaOtBu (0.82 g, 8.54 mmol, 2 eq) and Rac-BINAP (160 mg, 0.25 mmol, 0.06 eq). Reaction mixture was degassed with N₂ for 10 min then added Pd₂(dba)₃ (117 mg, 0.13 mmol, 0.03 eq). The reaction mixture was stirred at 100° C. for 16 h. After completion of reaction by TLC, reaction mixture was diluted with water (35 mL) and extracted with EtOAc (2×70 mL). The combined organic layer was dried over Na₂SO₄, filtered and concentrated under reduced pressure. The crude product was purified by reverse phase column chromatography [elution with 70% ACN in 0.1% FA in H₂O] to afford (S)-5-(3-((tert-butyldimethylsilyl)oxy)pyrrolidin-1-yl)-1-((2-(trimethylsilyl)ethoxy)methyl)-1H-indole-3-carbonitrile as a pale-yellow liquid (1.1 g, Yield: 54%). TLC system: EtOAc/Hexane (50:50), R_{f} value: ˜0.8; LCMS (m/z): 472.55 (M+H)⁺.

### Synthesis of(S)-5-(3-((tert-butyldimethylsilyl)oxy)pyrrolidin-1-yl)-N-hydroxy-1-((2-(trimethylsilyl)ethoxy)methyl)-1H-indole-3-carboximidamide

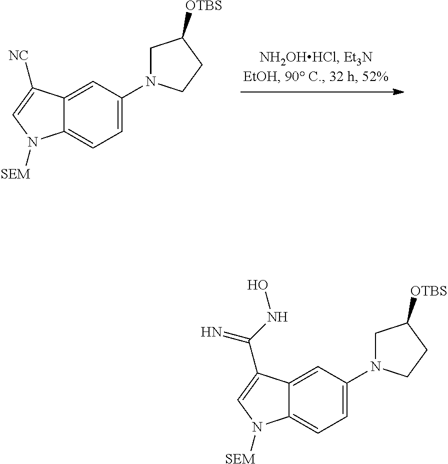 [SMILES: CC(C)(C)[Si](C)(C)O[C@H]1CCN(c2ccc3c(c2)c(C#N)cn3COCC[Si](C)(C)C)C1 ; CC(C)(C)[Si](C)(C)O[C@H]1CCN(c2ccc3c(c2)c(C(=N)NO)cn3COCC[Si](C)(C)C)C1]

To a stirred solution of(S)-5-(3-((tert-butyldimethylsilyl)oxy)pyrrolidin-1-yl)-1-((2-(trimethylsilyl)ethoxy)methyl)-1H-indole-3-carbonitrile (0.9 g, 1.90 mmol, 1.0 eq) in EtOH (10 mL) at room temperature, was added Et₃N (0.55 mL, 3.80 mmol, 2 eq) and NH₂OH·HCl (0.21 g, 3.04 mmol, 1.6 eq). The reaction mixture was stirred at 90° C. for 32 h. After completion of reaction by TLC, the reaction mixture was diluted with water (35 mL) and extracted with EtOAc (2×70 mL). The combined organic layer was dried over Na₂SO₄, filtered and concentrated under reduced pressure to afford crude product. The crude product was purified by silica gel (100-200 mesh) column chromatography [elution with 70% EtOAc/Hexane] to afford (S)-5-(3-((tert-butyldimethylsilyl)oxy)pyrrolidin-1-yl)-N-hydroxy-1-((2-(trimethylsilyl)ethoxy)methyl)-1H-indole-3-carboximidamide as a brown liquid (500 mg, Yield: 52%). TLC system: EtOAc/Hexane (50:50), R_{f} value: ˜0.2; LCMS (m/z): 505.4 (M+H)⁺.

### Synthesis of(S)-5-(3-((tert-butyldimethylsilyl)oxy)pyrrolidin-1-yl)-N-((ethoxycarbonyl)oxy)-1-((2-(trimethylsilyl)ethoxy)methyl)-1H-indole-3-carboximidamide

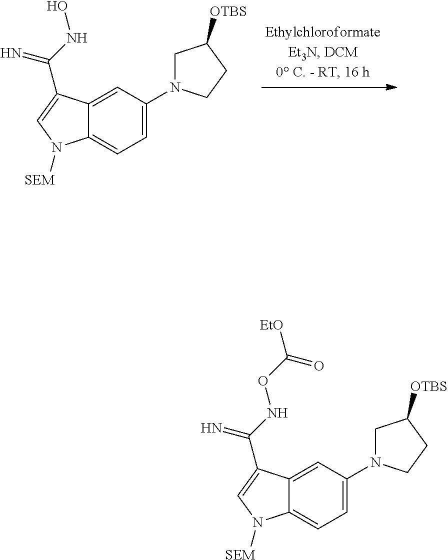 [SMILES: CC(C)(C)[Si](C)(C)O[C@H]1CCN(c2ccc3c(c2)c(C(=N)NO)cn3COCC[Si](C)(C)C)C1 ; CCOC(=O)ONC(=N)c1cn(COCC[Si](C)(C)C)c2ccc(N3CC[C@H](O[Si](C)(C)C(C)(C)C)C3)cc12]

To a stirred solution of(S)-5-(3-((tert-butyldimethylsilyl)oxy)pyrrolidin-1-yl)-N-hydroxy-1-((2-(trimethylsilyl)ethoxy)methyl)-1H-indole-3-carboximidamide (500 mg, 1.00 mmol, 1.0 eq) in DCM (5 mL, 10 Vol) at 0° C., was added Et₃N (0.18 mL, 1.30 mmol, 1.3 eq) and ethylchloroformate (0.12 mL, 1.20 mmol, 1.2 eq). The reaction mixture was stirred at room temperature for 16 h. After completion of reaction by TLC, the reaction mixture was quenched with NaHCO₃ solution (10 mL) and extracted with EtOAc (2×75 mL). The organic layer was dried over Na₂SO₄, filtered and concentrated to afford (S)-5-(3-((tert-butyldimethylsilyl)oxy)pyrrolidin-1-yl)-N-((ethoxycarbonyl)oxy)-1-((2-(trimethylsilyl)ethoxy)methyl)-1H-indole-3-carboximidamide as brown gummy solid (500 mg, Crude). TLC system: EtOAc/Hexane (50:50), R_{f} value: ˜0.8; LCMS (m/z): 577.5 (M+H)⁺.

### Synthesis of(S)-3-(5-(3-((tert-butyldimethylsilyl)oxy)pyrrolidin-1-yl)-1-((2-(trimethylsilyl)ethoxy)methyl)-1H-indol-3-yl)-1,2,4-oxadiazol-5(4H)-one

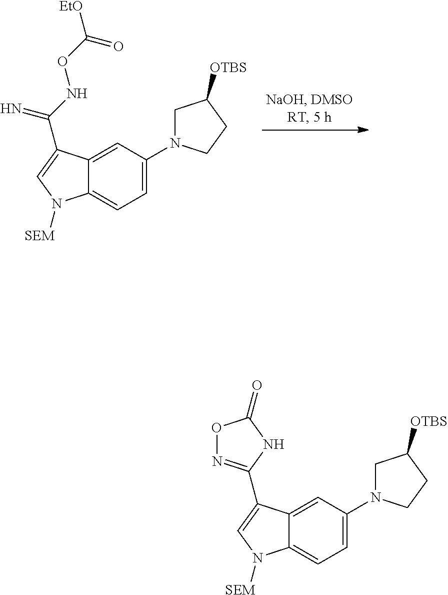 [SMILES: CCOC(=O)ONC(=N)c1cn(COCC[Si](C)(C)C)c2ccc(N3CC[C@H](O[Si](C)(C)C(C)(C)C)C3)cc12 ; CC(C)(C)[Si](C)(C)O[C@H]1CCN(c2ccc3c(c2)c(-c2noc(=O)[nH]2)cn3COCC[Si](C)(C)C)C1]

To a stirred solution of(S)-5-(3-((tert-butyldimethylsilyl)oxy)pyrrolidin-1-yl)-N-((ethoxycarbonyl)oxy)-1-((2-(trimethylsilyl)ethoxy)methyl)-1H-indole-3-carboximidamide (500 mg, 0.86 mmol, 1.0 eq) in DMSO (5 mL) at 0° C., was added NaOH (103 mg, 2.58 mmol, 3 eq) and stirred for 5 h at room temperature. After completion of reaction by TLC, the reaction mixture was diluted with water (20 mL), quenched with 2NHCl solution (2 mL), precipitated solid was filtered & dried to afford (S)-3-(5-(3-((tert-butyldimethylsilyl)oxy)pyrrolidin-1-yl)-1-((2-(trimethylsilyl)ethoxy)methyl)-1H-indol-3-yl)-1,2,4-oxadiazol-5(4H)-one as brown solid (350 mg, Crude). TLC system: EtOAc/Hexane (50:50), R_{f} value: ˜0.2; LCMS (m/z): 531.51 (M+H)⁺.

### Synthesis of(S)-3-(1-(hydroxymethyl)-5-(3-hydroxypyrrolidin-1-yl)-1H-indol-3-yl)-1,2,4-oxadiazol-5(4H)-one

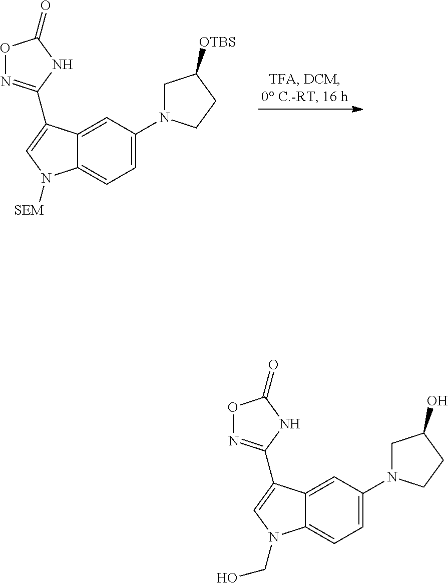 [SMILES: CC(C)(C)[Si](C)(C)O[C@H]1CCN(c2ccc3c(c2)c(-c2noc(=O)[nH]2)cn3COCC[Si](C)(C)C)C1 ; O=c1[nH]c(-c2cn(CO)c3ccc(N4CC[C@H](O)C4)cc23)no1]

To a stirred solution of(S)-3-(5-(3-((tert-butyldimethylsilyl)oxy)pyrrolidin-1-yl)-1-((2-(trimethylsilyl)ethoxy)methyl)-1H-indol-3-yl)-1,2,4-oxadiazol-5(4H)-one (300 mg, 0.56 mmol, 1.0 eq) in DCM (6 mL, 20 Vol) at 0° C., was added TFA (1.5 mL, 5 Vol) and stirred at room temperature for 16 h. After completion of reaction by TLC, reaction mixture concentrated, triturated with pentane (10 mL) to afford (S)-3-(1-(hydroxymethyl)-5-(3-hydroxypyrrolidin-1-yl)-1H-indol-3-yl)-1,2,4-oxadiazol-5(4H)-one as brown solid (300 mg, crude). TLC system: EtOAc (100%), R_{f} value: ˜0.2; LCMS (m/z): 317.2 (M+H).+

### Synthesis of(S)-3-(5-(3-hydroxypyrrolidin-1-yl)-1H-indol-3-yl)-1,2,4-oxadiazol-5(4H)-one

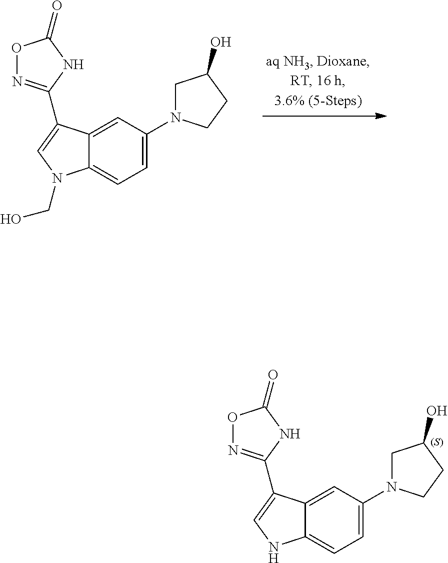

To a stirred solution of(S)-3-(1-(hydroxymethyl)-5-(3-hydroxypyrrolidin-1-yl)-1H-indol-3-yl)-1,2,4-oxadiazol-5(4H)-one (Crude 48%) (300 mg, 0.95 mmol, 1.0 eq) in 1,4-dioxane (3 mL) added aq NH₃ (3 mL) and stirred at room temperature for 16 h. Reaction mass was concentrated under reduced pressure, diluted with water (20 mL), solid filtered, washed with water & ether (10 mL), dried to provide crude product. The crude product was purified by reverse phase column chromatography [elution with 35% ACN in 0.1% FA in H₂O] and concentrated to afford semi pure compound. This was re-purified by Prep-HPLC, collected fractions were frozen and lyophilized to afford (S)-3-(5-(3-hydroxypyrrolidin-1-yl)-1H-indol-3-yl)-1,2,4-oxadiazol-5(4H)-one as off white solid (20 mg, Yield: 3.6%-5 steps). TLC system: EtOAc (100%), R_{f} value: ˜0.2; LCMS (m/z): 287.23 [M+H]⁺. ¹H NMR (400 MHz, DMSO-d₆) δ: 11.29 (s, 1H), 8.31 (s, 1H), 7.68 (d, J=2.8 Hz, 1H), 7.28 (d, J=8.8 Hz, 1H), 6.98 (d, J=2 Hz, 1H), 6.58 (dd, J=2.4 Hz, 8.8 Hz, 1H), 4.93 (br s, 1H), 4.42-4.40 (m, 1H), 3.51-3.40 (m, 1H), 3.37-3.31 (m, 1H), 3.28-3.23 (m, 1H), 3.09-3.06 (m, 1H), 2.10-2.05 (m, 1H), 1.92-1.89 (m, 1H).

Prep Conditions: COLUMN NAME: X-bridge C18 (250*19) mm 5 MICRON, Buffer MP-(A): 0.1% FA IN WATER MP-(B): 100% ACN Gradient (T/% B) 0/5, 13/35, 15/60,20/95, Flow Rate 12 ml/min, Solubility ACN, WATER.

### SYNTHETIC EXAMPLE 33

### Synthesis of Compound I-40

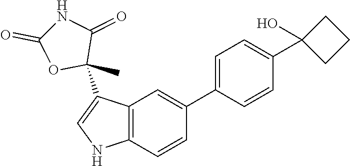 [SMILES: C[C@@]1(c2c[nH]c3ccc(-c4ccc(C5(O)CCC5)cc4)cc23)OC(=O)NC1=O]

### Synthesis of 2-(5-bromo-1H-indol-3-yl)-2-oxoacetamide

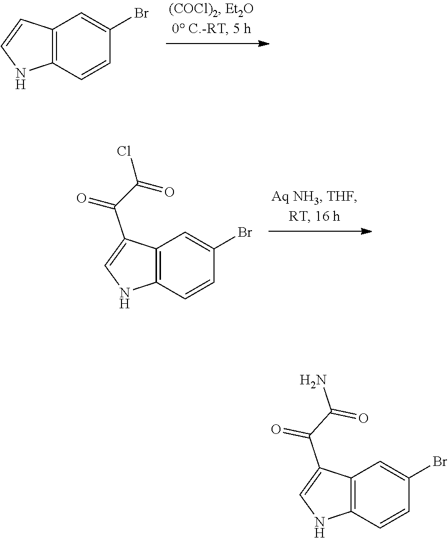 [SMILES: Brc1ccc2[nH]ccc2c1 ; O=C(Cl)C(=O)c1c[nH]c2ccc(Br)cc12 ; NC(=O)C(=O)c1c[nH]c2ccc(Br)cc12]

To a stirred solution of 5-bromo-1H-indole (5 g, 21.5 mmol, 1.0 eq) in diethyl ether (50 mL) at 0° C., added Oxalyl chloride (4.8 g, 38.2 mmol, 1.5 eq) and allowed to room temperature, stirred for 5 h. After completion of reaction by TLC, solid material was filtered, washed with n-pentane (50 mL) and dried under vacuum to afford 2-(5-bromo-1H-indol-3-yl)-2-oxoacetyl chloride as pale-yellow solid (6 g, Crude). TLC system: EtOAc/petroleum ether (20:80), R_{f} value: ˜0.8.

A solution of 2-(5-bromo-1H-indol-3-yl)-2-oxoacetyl chloride (6.0 g, 21.1 mmol, 1.0 eq) in THF (60 mL) at 0° C., NH₃ gas purged to the reaction mixture for 20 min, and stirred at room temperature for 16 h. After completion of reaction by TLC, solid material was filtered, washed with n-pentane (50 mL) and dried under vacuum to afford 2-(5-bromo-1H-indol-3-yl)-2-oxoacetamide as off white solid (4.2 g, Crude). TLC system: EtOAc/petroleum ether (50:50), R_{f} value: ^{˜}0.5, LCMS (m/z): 267.06 (M+H)⁺.

### Synthesis of 2-(5-bromo-1-tosyl-1H-indol-3-yl)-2-oxoacetamide

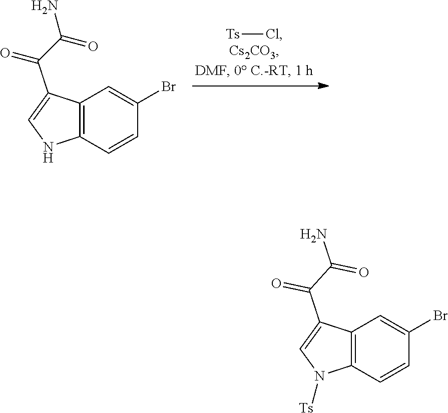 [SMILES: NC(=O)C(=O)c1c[nH]c2ccc(Br)cc12 ; Cc1ccc(S(=O)(=O)Cl)cc1 ; Cc1ccc(S(=O)(=O)n2cc(C(=O)C(N)=O)c3cc(Br)ccc32)cc1]

To a stirred solution of 2-(5-bromo-1H-indol-3-yl)-2-oxoacetamide (4.2 g, 15.8 mmol, 1 eq), in DMF (42 mL) at 0° C. was added Cs₂CO₃ (15.4 g, 47.4 mmol, 3 eq). After 5 min, Ts-Cl (6 g, 31.5 mmol, 3.0 eq) was added at 0° C. and stirred at room temperature for 1 h. After completion of reaction by TLC, diluted with water (50 mL) and extracted with EtOAc (2×100 mL). Organic layer dried over anhydrous Na₂SO₄ and concentrated under reduced pressure to afford 2-(5-bromo-1-tosyl-1H-indol-3-yl)-2-oxoacetamide as off white solid (6.2 g, yield: 93%). TLC system: EtOAc/petroleum ether (50:50), R_{f} value: ^{˜}0.8; LCMS (m/z): 421.1 (M+H)⁺. ¹H NMR (400 MHz, CDCl₃) δ: 9.36 (s, 1H), 8.53 (d, J=2.0 Hz, 1H), 7.90-7.87 (m, 3H), 7.54-7.51 (m, 1H), 7.32 (d, J=8.0 Hz, 2H), 7.20 (s, 1H), 5.59 (s, 1H), 2.41 (s, 3H).

### Synthesis of 2-(5-bromo-1-tosyl-1H-indol-3-yl)-2-hydroxypropanamide

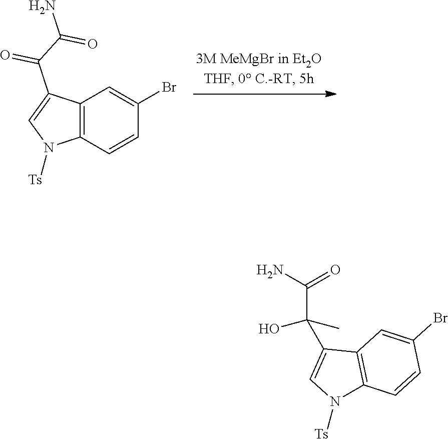 [SMILES: Cc1ccc(S(=O)(=O)n2cc(C(=O)C(N)=O)c3cc(Br)ccc32)cc1 ; Cc1ccc(S(=O)(=O)n2cc(C(C)(O)C(N)=O)c3cc(Br)ccc32)cc1]

To a stirred solution of 2-(5-bromo-1-tosyl-1H-indol-3-yl)-2-oxoacetamide (3 g, 7.14 mmol, 1 eq) in THF (30 mL) at 0° C., was added MeMgBr (3.0 M in Et₂O) (3.6 mL, 10.7 mmol, 1.5 eq) and stirred at room temperature for 5 h. After completion of reaction by TLC, quenched with NH₄Cl solution (20 mL) and extracted with 10% MeOH/DCM (2×100 mL). The combined organic layer was dried over anhydrous Na₂SO₄, filtered and concentrated under reduced pressure. The crude compound was triturated with diethyl ether (20 mL) to afford 2-(5-bromo-1-tosyl-1H-indol-3-yl)-2-hydroxypropanamide as off white solid (1.5 g, yield: 48%). TLC system: EtOAc/Hexane (50:50), R_{f} value: ^{˜}0.3; LCMS (m/z): 419.16 (M−17)⁺:

### Synthesis of 5-(5-bromo-1-tosyl-1H-indol-3-yl)-5-methyloxazolidine-2,4-dione

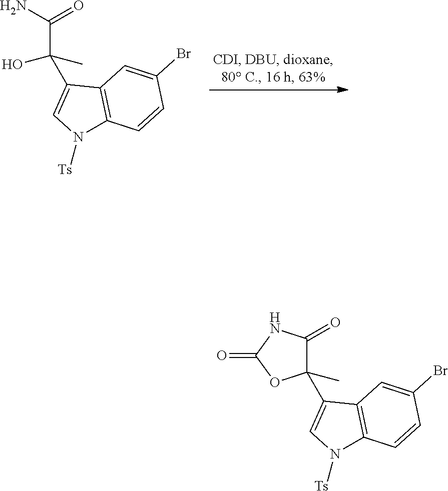 [SMILES: Cc1ccc(S(=O)(=O)n2cc(C(C)(O)C(N)=O)c3cc(Br)ccc32)cc1 ; Cc1ccc(S(=O)(=O)n2cc(C3(C)OC(=O)NC3=O)c3cc(Br)ccc32)cc1]

To a stirred solution of 2-(5-bromo-1-tosyl-1H-indol-3-yl)-2-hydroxypropanamide (1.5 g, 3.44 mmol, 1.0 eq) in dioxane (15 mL, 10 Vol) at room temperature, was added DBU (1.02 mL, 6.80 mmol, 2 eq), CDI (1.1 g, 6.80 mmol, 2 eq) and stirred at 80° C. for 16 h. After completion of reaction by TLC, reaction mixture was diluted with ice cold water (20 mL) and acidified with 2N aq. HCl (5 mL), stirred at room temperature for 20 min. Solid was filtered, washed with n-pentane (10 mL) and dried to afford 5-(5-bromo-1-tosyl-1H-indol-3-yl)-5-methyloxazolidine-2,4-dione as brown solid (1 g, yield: 63%). TLC system: EtOAc/Hexane (50:50), R_{f} value: ˜0.8; LCMS (m/z): 461.03 (M−H)⁻:

### Synthesis of 5-(5-(4-(1-hydroxycyclobutyl)phenyl)-1-tosyl-1H-indol-3-yl)-5-methyloxazolidine-2,4-dione

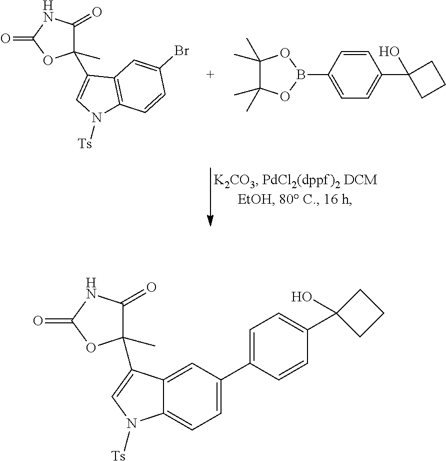 [SMILES: Cc1ccc(S(=O)(=O)n2cc(C3(C)OC(=O)NC3=O)c3cc(Br)ccc32)cc1 ; CC1(C)OB(c2ccc(C3(O)CCC3)cc2)OC1(C)C ; Cc1ccc(S(=O)(=O)n2cc(C3(C)OC(=O)NC3=O)c3cc(-c4ccc(C5(O)CCC5)cc4)ccc32)cc1]

In a sealed tube to a degassed solution of 5-(5-bromo-1-tosyl-1H-indol-3-yl)-5-methyloxazolidine-2,4-dione (300 mg, 0.65 mmol, 1.0 eq) and 1-(4-(4,4,5,5-tetramethyl-1,3,2-dioxaborolan-2-yl)phenyl)cyclobutan-1-ol (444 mg, 1.62 mmol, 2.5 eq) in EtOH (3 mL) at room temperature, was added K₂CO₃ (268 mg, 1.94 mmol, 3.0 eq) followed by Pd(dppf)Cl₂·DCM (53 mg, 0.065 mmol, 0.1 eq) and stirred at 80° C. for 16 h. After completion of reaction by TLC, reaction mixture was evaporated and purified by silica column chromatography [with a gradient elution of 5-10% MeOH/DCM] to afford 5-(5-(4-(1-hydroxycyclobutyl)phenyl)-1-tosyl-1H-indol-3-yl)-5-methyloxazolidine-2,4-dione as brown gummy (250 mg, semi pure). TLC system: EtOAc/Hexane (50:50), R_{f} value: ˜0.3; LCMS (m/z): 529.2 (M−H)

### Synthesis of 5-(5-(4-(1-hydroxycyclobutyl)phenyl)-1H-indol-3-yl)-5-methyloxazolidine-2,4-dione

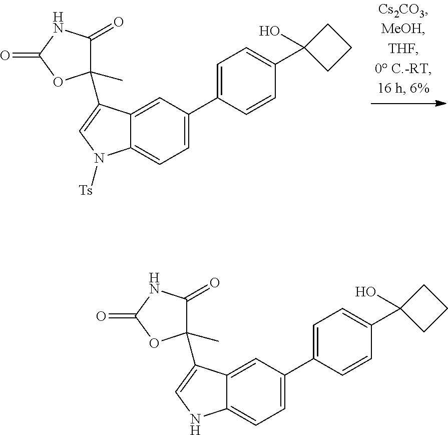 [SMILES: Cc1ccc(S(=O)(=O)n2cc(C3(C)OC(=O)NC3=O)c3cc(-c4ccc(C5(O)CCC5)cc4)ccc32)cc1 ; CC1(c2c[nH]c3ccc(-c4ccc(C5(O)CCC5)cc4)cc23)OC(=O)NC1=O]

To a stirred solution of 5-(5-(4-(1-hydroxycyclobutyl)phenyl)-1-tosyl-1H-indol-3-yl)-5-methyloxazolidine-2,4-dione (210 mg, 0.40 mmol, 1 eq) (Crude) in THF:MeOH (1:1 ratio) (2 mL) at 0° C. was added Cs₂CO₃ (776 mg, 2.40 mmol, 6 eq) and stirred at room temperature for 16 h. After completion of reaction by TLC, reaction mass was concentrated and purified by reverse phase column chromatography [elution with 35-40% ACN in 0.1% FA in H₂O], lyophilized to afford 5-(5-(4-(1-hydroxycyclobutyl)phenyl)-1H-indol-3-yl)-5-methyloxazolidine-2,4-dione as off white solid (14 mg, Yield: 5.7% (2-Steps). TLC system: EtOAc (100%), R_{f} value: ˜0.6; LCMS (m/z): 375.0 [M−H]⁻. ¹H NMR (400 MHz, DMSO-d₆) δ: 12.80-11.90 (br, 1H), 11.46 (s, 1H), 7.60-7.50 (m, 7H), 7.45-7.43 (m, 1H), 5.50 (s, 1H), 2.45-2.40 (m, 2H), 2.33-2.28 (m, 2H), 1.97 (s, 3H), 1.95-1.92 (m, 1H), 1.68-1.66 (m, 1H).

### SYNTHETIC EXAMPLE 34

### Synthesis of Compound I-43

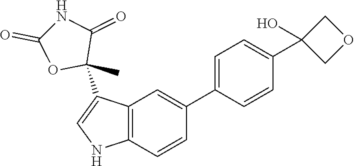 [SMILES: C[C@@]1(c2c[nH]c3ccc(-c4ccc(C5(O)COC5)cc4)cc23)OC(=O)NC1=O]

### Synthesis of 5-(5-(4-(3-hydroxyoxetan-3-yl)phenyl)-1-tosyl-1H-indol-3-yl)-5-methyloxazolidine-2,4-dione

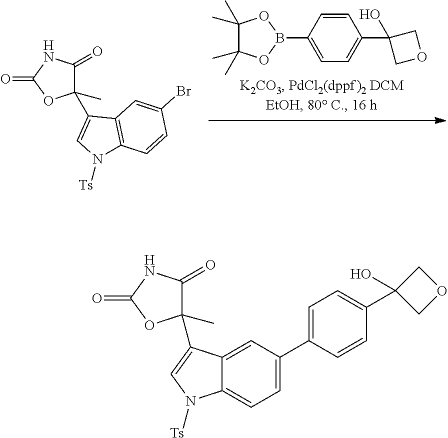 [SMILES: Cc1ccc(S(=O)(=O)n2cc(C3(C)OC(=O)NC3=O)c3cc(Br)ccc32)cc1 ; CC1(C)OB(c2ccc(C3(O)COC3)cc2)OC1(C)C ; Cc1ccc(S(=O)(=O)n2cc(C3(C)OC(=O)NC3=O)c3cc(-c4ccc(C5(O)COC5)cc4)ccc32)cc1]

In a sealed tube to a degassed solution of 5-(5-bromo-1-tosyl-1H-indol-3-yl)-5-methyloxazolidine-2,4-dione (150 mg, 0.32 mmol, 1.0 eq) and 3-(4-(4,4,5,5-tetramethyl-1,3,2-dioxaborolan-2-yl)phenyl)oxetan-3-ol (224 mg, 0.80 mmol, 2.5 eq) in EtOH (2 mL, 10 Vol) at room temperature was added K₂CO₃ (134 mg, 0.98 mmol, 3.0 eq) followed by Pd(dppf)Cl₂·DCM (26 mg, 0.03 mmol, 0.1 eq) and stirred at 80° C. for 16 h. After completion of reaction by TLC, reaction mixture was concentrated under reduced pressure and purified by reverse phase column chromatography [elution with 40-46% ACN in 0.1% FA in H₂O] to afford 5-(5-(4-(3-hydroxyoxetan-3-yl)phenyl)-1-tosyl-1H-indol-3-yl)-5-methyloxazolidine-2,4-dione as a off brown solid (180 mg, semi-pure). TLC system: EtOAC/Hexane (50:50), R_{f} value: ˜0.1; LCMS (m/z) (>50% Purity): 531.22 (M+H)⁺

### Synthesis of 5-(5-(4-(3-hydroxyoxetan-3-yl)phenyl)-1H-indol-3-yl)-5-methyloxazolidine-2,4-dione

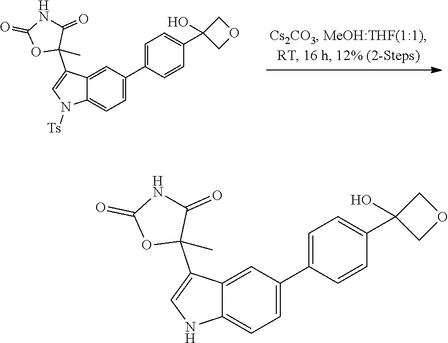 [SMILES: Cc1ccc(S(=O)(=O)n2cc(C3(C)OC(=O)NC3=O)c3cc(-c4ccc(C5(O)COC5)cc4)ccc32)cc1 ; CC1(c2c[nH]c3ccc(-c4ccc(C5(O)COC5)cc4)cc23)OC(=O)NC1=O]

To a stirred solution of 5-(5-(4-(3-hydroxyoxetan-3-yl)phenyl)-1-tosyl-1H-indol-3-yl)-5-methyloxazolidine-2,4-dione (170 mg, 0.32 mmol, 1 eq) in THF:MeOH (1:1 ratio) (1.7 mL, 10 Vol) at 0° C. was added Cs₂CO₃ (620 mg, 1.92 mmol, 6 eq) and stirred at room temperature for 16 h. After completion of reaction by TLC, reaction mixture was evaporated, diluted with water (20 mL) and extracted with Ethyl acetate (2×50 mL). Organic layer was dried over Na₂SO₄, evaporated and purified by reverse phase column chromatography [elution with 40-45% ACN in 0.1% FA in H₂O] to afford 5-(5-(4-(3-hydroxyoxetan-3-yl)phenyl)-1H-indol-3-yl)-5-methyloxazolidine-2,4-dione as off white solid (15 mg, yield: 12% (2-steps)). TLC system: EtOAc (100), R_{f} value: ^{˜}0.1; LCMS (m/z): 377.23 [M−H]⁻; ¹H NMR (400 MHz, DMSO-d₆) δ: 12.24 (s, 1H), 11.49 (s, 1H), 7.69-7.60 (m, 6H), 7.53-7.45 (m, 2H), 6.36 (s, 1H), 4.80 (d, J=6.6 Hz, 2H), 4.74 (d, J=6.6 Hz, 2H), 1.98 (s, 3H).

### SYNTHETIC EXAMPLE 35

### Synthesis of Compound I-44

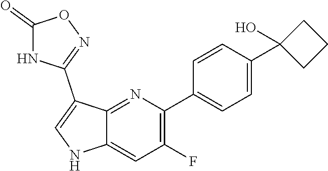 [SMILES: O=c1[nH]c(-c2c[nH]c3cc(F)c(-c4ccc(C5(O)CCC5)cc4)nc23)no1]

### Synthesis of 5-fluoro-2-((trimethylsilyl) ethynyl) pyridin-3-amine

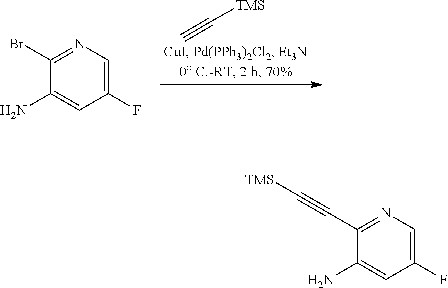

To a stirred solution of 2-bromo-5-fluoropyridin-3-amine (5 g, 26.17 mmol, 1.0 eq) in Et₃N (75 mL) at 0° C., was added CuI (250 mg, 1.31 mmol, 0.05 eq), Pd(PPh₃)Cl₂ (940 mg, 1.31 mmol, 0.05 eq), ethynyltrimethylsilane (3.0 g, 31.41 mmol, 1.2 eq) and stirred at room temperature for 2 h. After completion of reaction by TLC, volatiles removed under vacuum and purified by silica gel [60-120] column chromatography [elution with 10% EtOAc in petroleum ether] to afford 5-fluoro-2-((trimethylsilyl) ethynyl) pyridin-3-amine as yellow solid (3.8 g, Yield: 70%). TLC system: EtOAc/Hexane (20:80), R_{f} value: ˜0.5; ¹H NMR (400 MHz, CDCl₃) δ: 7.83 (d, J=2.4 Hz, 1H), 6.73 (dd, J=2.4 Hz, 9.6 Hz, 1H), 4.39 (bs, 2H), 0.28 (s, 9H).

### Synthesis of 6-fluoro-1H-pyrrolo[3,2-b]pyridine

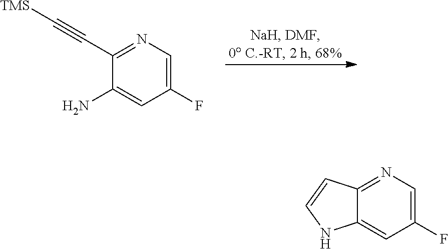 [SMILES: C[Si](C)(C)C#Cc1ncc(F)cc1N ; Fc1cnc2cc[nH]c2c1]

To a stirred solution of 5-fluoro-2-((trimethylsilyl) ethynyl) pyridin-3-amine (3.8 g, 18.3 mmol, 1.0 eq) in DMF (40 mL) at 0° C., was added NaH (1.75 g, 73.2 mmol, 4.0 eq) portion wise and stirred at room temperature for 2 h. After completion of reaction by TLC, quenched with ice cold water (100 mL) and extracted with EtOAc (2×50 mL). The combined organic layer was washed with brine (100 mL), dried over Na₂SO₄, filtered and concentrated under reduced pressure. Obtained crude was purified by silica gel (100-200) column chromatography [elution with 20% EtOAc in petroleum ether] to afford 6-fluoro-1H-pyrrolo[3,2-b]pyridine as yellow solid (1.7 g, Yield: 68%). TLC system: EtOAc/Hexane (30:70), R_{f} value: ˜0.3; LCMS (m/z): 137.2 (M+H)⁺; ¹H NMR (400 MHz, CDCl₃) δ: 8.59 (bs, 1H), 8.40-8.39 (m, 1H), 7.46-7.41 (m, 2H), 6.76-6.74 (m, 1H).

### Synthesis of 6-fluoro-1H-pyrrolo[3,2-b]pyridine 4-oxide

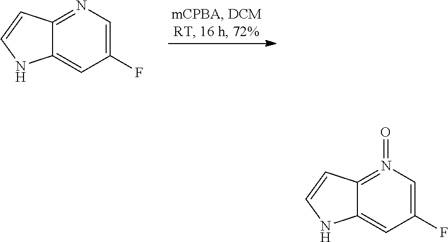 [SMILES: Fc1cnc2cc[nH]c2c1 ; [O-][n+]1cc(F)cc2[nH]ccc21]

To a stirred solution of 6-fluoro-1H-pyrrolo[3,2-b]pyridine (1.7 g, 12.4 mmol, 1.0 eq) in DCM (170 mL) at room temperature, was added m-CPBA (4.27 g, 24.8 mmol, 2.0 eq) and stirred at room temperature for 16 h. After completion of reaction by TLC, the reaction mixture was cooled to 0° C., precipitation observed. Precipitated solid was filtered, washed with DCM (500 mL) dried under vacuum to afford 6-fluoro-1H-pyrrolo[3,2-b]pyridine 4-oxide as pale brown solid (1.35 g, Yield: 72%). TLC system: MeOH/DCM (10:90), R_{f} value: ˜0.2; LCMS (m/z): 153.1 (M+H)⁺; ¹H NMR (400 MHz, CDCl₃) δ: 11.86 (s, 1H), 8.32-8.30 (m, 1H), 7.58-7.56 (m, 1H), 7.47-7.45 (m, 1H), 6.66-6.65 (m, 1H).

### Synthesis of methyl 5-chloro-6-fluoro-1H-pyrrolo[3,2-b]pyridine-1-carboxylate

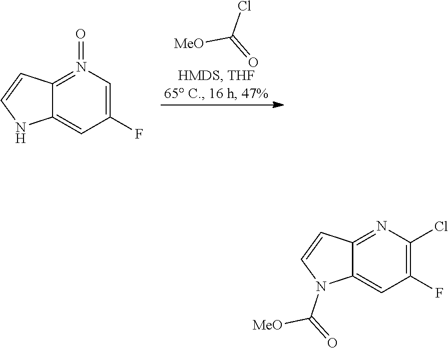 [SMILES: [O-][n+]1cc(F)cc2[nH]ccc21 ; COC(=O)Cl ; COC(=O)n1ccc2nc(Cl)c(F)cc21]

To a stirred solution of 6-fluoro-1H-pyrrolo[3,2-b]pyridine 4-oxide (2.7 g, 17.76 mmol, 1.0 eq) in THF (27 mL) at room temperature, was added HMDS (3.14 g, 19.54 mmol, 1.1 eq) followed by methyl carbonochloridate (27 mL, 10 Vol) and stirred at 65° C. for 16 h. After completion of reaction by TLC, diluted with water (100 mL) and extracted with EtOAc (2× 100 mL). Combined organic layer was dried over anhydrous Na₂SO₄, filtered and concentrated under reduced pressure. Obtained crude was purified by silica gel [100-200] column chromatography [elution with 10% EtOAc in petroleum ether] to afford methyl 5-chloro-6-fluoro-1H-pyrrolo[3,2-b]pyridine-1-carboxylate as white solid (1.9 g, Yield: 47%). TLC system: EtOAc/Hexane (20:80), R_{f} value: ˜0.5; LCMS (m/z): 229.0 (M+H)⁺; ¹H NMR (400 MHz, CDCl₃) δ: 8.25 (brs, 1H), 7.85-7.84 (m, 1H), 6.75 (dd, J=0.4 & 4 Hz, 1H), 4.09 (s, 3H).

### Synthesis of 5-chloro-6-fluoro-1H-pyrrolo[3,2-b]pyridine

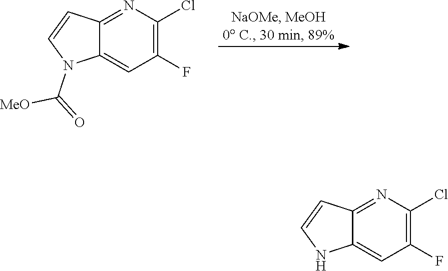 [SMILES: COC(=O)n1ccc2nc(Cl)c(F)cc21 ; Fc1cc2[nH]ccc2nc1Cl]

To a stirred solution of methyl 5-chloro-6-fluoro-1H-pyrrolo[3,2-b]pyridine-1-carboxylate (1.9 g, 8.29 mmol, 1.0 eq) in MeOH (19 mL) at 0° C., was added NaOMe (1.34 g, 24.89 mmol, 3.0 eq) and stirred at same temperature for 30 min. After completion of reaction by TLC, the reaction mixture was quenched with 2N HCl solution (20 mL), diluted with water (30 mL) and extracted with EtOAc (2×50 mL). The combined organic layer was dried over anhydrous Na₂SO₄, filtered and concentrated under reduced pressure. Crude product was triturated with n-pentane (50 mL), decanted and dried under vacuum to afford 5-chloro-6-fluoro-1H-pyrrolo[3,2-b]pyridine as off white solid (1.25 g, Yield: 89%). TLC system: EtOAc/Hexane (30:70), R_{f} value: ˜0.3; LCMS (m/z): 171.0 (M+H)⁺.

### Synthesis of 5-chloro-6-fluoro-1H-pyrrolo[3,2-b]pyridine-3-carbaldehyde

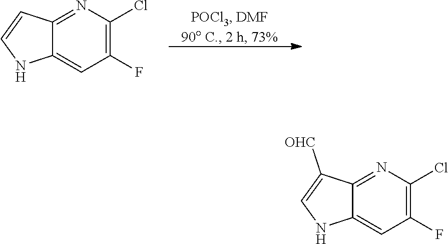 [SMILES: Fc1cc2[nH]ccc2nc1Cl ; O=Cc1c[nH]c2cc(F)c(Cl)nc12]

To a stirred solution of POCl₃ (1.79 g, 11.7 mmol, 2.0 eq) in DMF (897 mg, 12.3 mmol, 2.1 eq) at room temperature, was added a solution of 5-chloro-6-fluoro-1H-pyrrolo[3,2-b]pyridine (1.0 g, 5.85 mmol, 1.0 eq) in DMF (1 mL). Reaction mixture was stirred at 90° C. for 2 h. After completion of reaction by TLC, treated with 1N NaOH solution (10 mL), diluted with water (30 mL) and extracted with EtOAc (2×50 mL) followed by 10% MeOH in DCM (50 mL). Combined organic layer dried over anhydrous Na₂SO₄, filtered and concentrated under reduced pressure. Crude product was triturated with n-pentane (30 mL), decanted and dried under vacuum to afford 5-chloro-6-fluoro-1H-pyrrolo[3,2-b]pyridine-3-carbaldehyde as off white solid (850 mg, Yield: 73%). TLC system: EtOAc/Hexane (50:50), R_{f} value: ˜0.3; LCMS (m/z): 199.02 (M+H)⁺.

### Synthesis of 5-chloro-6-fluoro-1H-pyrrolo[3,2-b]pyridine-3-carbonitrile

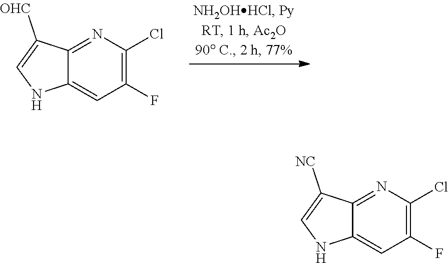 [SMILES: O=Cc1c[nH]c2cc(F)c(Cl)nc12 ; N#Cc1c[nH]c2cc(F)c(Cl)nc12]

To a stirred solution of 5-chloro-6-fluoro-1H-pyrrolo[3,2-b]pyridine-3-carbaldehyde (800 mg, 4.04 mmol, 1.0 eq) in pyridine (4 mL) at room temperature, was added NH₂OH·HCl (307 g, 4.44 mmol, 1.1 eq) and stirred for 1 h. Then Ac₂O (824 mg, 8.08 mmol, 2.0 eq) was added to reaction mixture and stirred at 90° C. for 2 h. After completion of reaction by TLC, diluted with water (20 mL) and extracted with EtOAc (2×20 mL). The combined organic layer was washed with 2N HCl solution (30 mL), dried over anhydrous Na₂SO₄, filtered and concentrated under reduced pressure to afford 5-chloro-6-fluoro-1H-pyrrolo[3,2-b]pyridine-3-carbonitrile as yellow solid (600 mg, Yield: 77%). TLC system: EtOAc/Hexane (50:50), R_{f} value: ˜0.4; LCMS (m/z): 195.99 (M+H)⁺.

### Synthesis of 5-chloro-6-fluoro-1-((2-(trimethylsilyl)ethoxy)methyl)-1H-pyrrolo[3,2-b]pyridine-3-carbonitrile

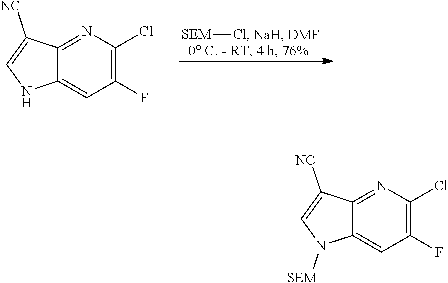 [SMILES: N#Cc1c[nH]c2cc(F)c(Cl)nc12 ; C[Si](C)(C)CCOCCl ; C[Si](C)(C)CCOCn1cc(C#N)c2nc(Cl)c(F)cc21]

To a stirred solution of 5-chloro-6-fluoro-1H-pyrrolo[3,2-b]pyridine-3-carbonitrile (550 mg, 2.82 mmol, 1.0 eq) in DMF (5.5 mL) at 0° C., was added NaH (226 mg, 5.64 mmol, 2.0 eq) and stirred for 30 min. Then SEM-Cl (1.17 g, 7.05 mmol, 2.5 eq) was added to reaction mixture and stirred at room temperature for 4 h. After completion of reaction by TLC, the reaction mixture was quenched with ice cold water (10 mL) and extracted with EtOAc (2×25 mL). The combined organic layer was washed with brine solution (20 mL), dried over Na₂SO₄, filtered and concentrated under reduced pressure. The crude product was purified by silica gel [60-120] column chromatography [elution with 10% EtOAc in petroleum ether] to afford 5-chloro-6-fluoro-1-((2-(trimethylsilyl)ethoxy)methyl)-1H-pyrrolo[3,2-b]pyridine-3-carbonitrile as yellow solid (650 mg, Yield: 76%). TLC system: EtOAc/Hexane (30:70), R_{f} value: ˜0.5; LCMS (m/z): 257.0 (M+H)⁺; ¹H NMR (400 MHz, CDCl₃) δ: 7.91 (s, 1H), 7.73 (d, J=8.0 Hz, 1H), 5.51 (s, 2H), 3.54-3.49 (m, 2H), 0.96-0.91 (m, 2H), −0.01 (s, 9H).

### Synthesis of 6-fluoro-5-(4-(1-hydroxycyclobutyl)phenyl)-1-((2-(trimethylsilyl)ethoxy)methyl)-1H-pyrrolo[3,2-b]pyridine-3-carbonitrile

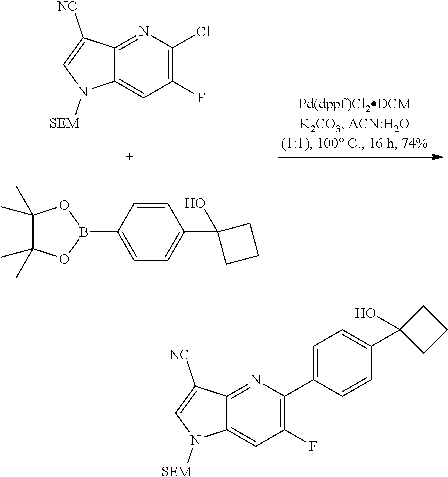 [SMILES: C[Si](C)(C)CCOCn1cc(C#N)c2nc(Cl)c(F)cc21 ; CC1(C)OB(c2ccc(C3(O)CCC3)cc2)OC1(C)C ; C[Si](C)(C)CCOCn1cc(C#N)c2nc(-c3ccc(C4(O)CCC4)cc3)c(F)cc21]

In a sealed tube, to a degassed solution of 5-chloro-6-fluoro-1-((2-(trimethylsilyl)ethoxy)methyl)-1H-pyrrolo[3,2-b]pyridine-3-carbonitrile (450 mg, 1.38 mmol, 1.0 eq) and 1-(4-(4,4,5,5-tetramethyl-1,3,2-dioxaborolan-2-yl)phenyl)cyclobutan-1-ol (570 mg, 2.07 mmol, 1.5 eq) in ACN:H₂O (1:1, 5 mL) at room temperature, was added K₂CO₃ (1.32 g, 4.14 mmol, 3.0 eq), Pd(dppf)Cl₂·DCM (113 mg, 0.14 mmol, 0.1 eq) and stirred at 100° C. for 16 h. After completion of reaction by TLC, reaction mixture was diluted with water (15 mL) and extracted with EtOAc (2×15 mL). The combined organic layer was dried over Na₂SO₄, filtered and concentrated under reduced pressure. The crude product was purified by silica gel [60-120] column chromatography [elution with 25% EtOAc in petroleum ether] to afford 6-fluoro-5-(4-(1-hydroxycyclobutyl)phenyl)-1-((2-(trimethylsilyl)ethoxy)methyl)-1H-pyrrolo[3,2-b]pyridine-3-carbonitrile as yellow gummy (550 mg, Yield: 74%). TLC system: EtOAc/Hexane (50:50), R_{f} value: ˜0.3; LCMS (m/z): 438.31 (M+H)⁺; ¹H NMR (400 MHz, CDCl₃) δ: 8.11-8.08 (m, 2H), 7.90 (s, 1H), 7.71-7.65 (m, 3H), 5.51 (s, 2H), 3.56-3.52 (m, 2H), 2.68-2.61 (m, 2H), 2.48-2.41 (m, 2H), 2.09-2.06 (m, 1H), 1.78-1.75 (m, 1H), 0.97-0.90 (m, 2H), 0.09 (s, 9H).

### Synthesis of (Z)-6-fluoro-N′-hydroxy-5-(4-(1-hydroxycyclobutyl)phenyl)-1-((2-(trimethylsilyl)ethoxy)methyl)-1H-pyrrolo[3,2-b]pyridine-3-carboximidamide

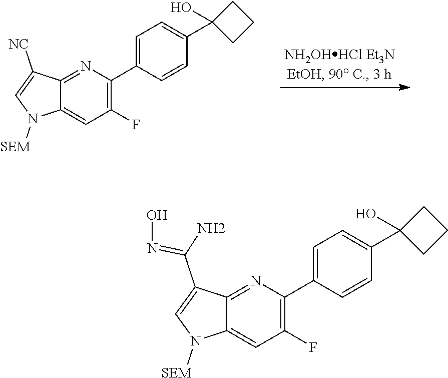 [SMILES: C[Si](C)(C)CCOCn1cc(C#N)c2nc(-c3ccc(C4(O)CCC4)cc3)c(F)cc21 ; C[Si](C)(C)CCOCn1cc(/C(N)=N/O)c2nc(-c3ccc(C4(O)CCC4)cc3)c(F)cc21]

To a stirred solution of 6-fluoro-5-(4-(1-hydroxycyclobutyl)phenyl)-1-((2-(trimethylsilyl)ethoxy)methyl)-1H-pyrrolo[3,2-b]pyridine-3-carbonitrile (500 mg, 1.14 mmol, 1.0 eq) in EtOH (5 mL) at room temperature, was added Et₃N (230 mg, 2.28 mmol, 2.0 eq) and NH₂OH·HCl (157 mg, 2.28 mmol, 2.0 eq). The reaction mixture was stirred at 90° C. for 3 h. After completion of reaction by TLC, the reaction mixture was diluted with water (10 mL) and extracted with 10% MeOH in DCM (2× 10 mL). The combined organic layer was dried over Na₂SO₄, filtered and concentrated under reduced pressure to afford (Z)-6-fluoro-N′-hydroxy-5-(4-(1-hydroxycyclobutyl)phenyl)-1-((2-(trimethylsilyl)ethoxy)methyl)-1H-pyrrolo[3,2-b]pyridine-3-carboximidamide as yellow gum (500 mg, Yield: Crude). TLC system: EtOAc/Hexane (100:00), R_{f} value: ˜0.1; LCMS (m/z): 471.3 (M+H)⁺.

### Synthesis of ethyl (Z)-((6-fluoro-5-(4-(1-hydroxycyclobutyl)phenyl)-1-((2-(trimethylsilyl)ethoxy)methyl)-1H-pyrrolo[3,2-b]pyridin-3-yl)(hydroxyimino)methyl)carbamate

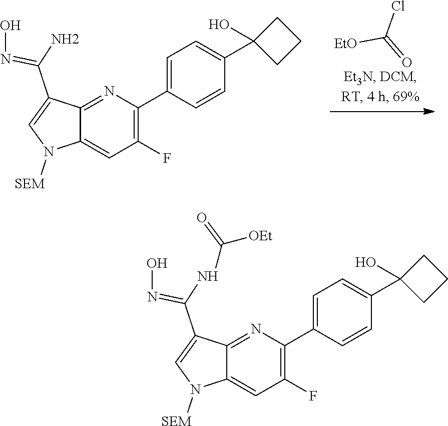 [SMILES: C[Si](C)(C)CCOCn1cc(/C(N)=N/O)c2nc(-c3ccc(C4(O)CCC4)cc3)c(F)cc21 ; CCOC(=O)Cl ; CCOC(=O)N/C(=N\O)c1cn(COCC[Si](C)(C)C)c2cc(F)c(-c3ccc(C4(O)CCC4)cc3)nc12]

To a stirred solution of (Z)-6-fluoro-N′-hydroxy-5-(4-(1-hydroxycyclobutyl)phenyl)-1-((2-(trimethylsilyl)ethoxy)methyl)-1H-pyrrolo[3,2-b]pyridine-3-carboximidamide (500 mg, 1.06 mmol, 1.0 eq) in DCM (5 mL) at 0° C., was added Et₃N (139 mg, 1.38 mmol, 1.3 eq) and ethylchloroformate (138 mg, 1.27 mmol, 1.2 eq). The reaction mixture was stirred at room temperature for 4 h. After completion of reaction by TLC, the reaction mixture was quenched with water (20 mL) and extracted with DCM (2×20 mL). The combined organic layer was dried over Na₂SO₄, filtered and concentrated to afford crude product. The crude product was purified by silica gel [100-200] column chromatography [elution with 20% EtOAc in petroleum ether] to afford ethyl (Z)-((6-fluoro-5-(4-(1-hydroxycyclobutyl)phenyl)-1-((2-(trimethylsilyl)ethoxy)methyl)-1H-pyrrolo[3,2-b]pyridin-3-yl)(hydroxyimino)methyl)carbamate as yellow gum (400 mg, Yield: 69%). TLC system: EtOAc/Hexane (30:70), R_{f} value: ˜0.4; LCMS (m/z): 543.48 (M+H)⁺; ¹H NMR (400 MHz, CDCl₃) δ: 8.12 (s, 1H), 7.96-7.94 (m, 2H), 7.69-7.64 (m, 3H), 5.46 (s, 2H), 4.35 (q, J=7.2 Hz, 2H), 3.49 (t, J=8.0 Hz, 2H), 2.67-2.61 (m, 2H), 2.47-2.39 (m, 2H), 2.09-2.05 (m, 1H), 1.79-1.73 (m, 1H), 1.38 (t, J=7.2 Hz, 3H), 0.93-0.86 (m, 2H), −0.03 (s, 9H).

### Synthesis of 3-(6-fluoro-5-(4-(1-hydroxycyclobutyl)phenyl)-1-((2-(trimethylsilyl)ethoxy)methyl)-1H-pyrrolo[3,2-b]pyridin-3-yl)-1,2,4-oxadiazol-5(4H)-one

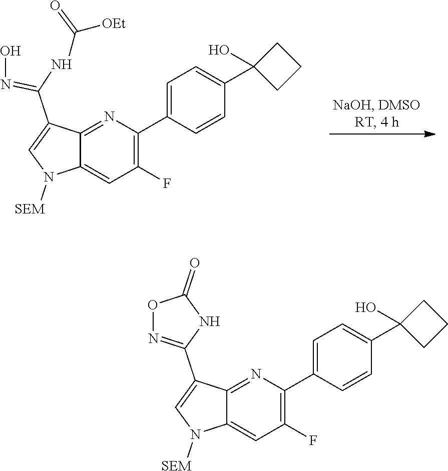 [SMILES: CCOC(=O)N/C(=N\O)c1cn(COCC[Si](C)(C)C)c2cc(F)c(-c3ccc(C4(O)CCC4)cc3)nc12 ; C[Si](C)(C)CCOCn1cc(-c2noc(=O)[nH]2)c2nc(-c3ccc(C4(O)CCC4)cc3)c(F)cc21]

To a stirred solution of ethyl (Z)-((6-fluoro-5-(4-(1-hydroxycyclobutyl)phenyl)-1-((2-(trimethylsilyl)ethoxy)methyl)-1H-pyrrolo[3,2-b]pyridin-3-yl)(hydroxyimino)methyl)carbamate (400 mg, 0.74 mmol, 1.0 eq) in DMSO (1.5 mL) at room temperature, was added NaOH (36 mg, 0.88 mmol, 1.2 eq) and reaction mixture was stirred at room temperature for 4 h. After completion of reaction by TLC, the reaction mixture was quenched with 2N aq. HCl solution (10 mL), precipitation observed. Solid filtered, washed with n-pentane (20 mL) and dried under vacuum to afford 3-(6-fluoro-5-(4-(1-hydroxycyclobutyl)phenyl)-1-((2-(trimethylsilyl)ethoxy)methyl)-1H-pyrrolo[3,2-b]pyridin-3-yl)-1,2,4-oxadiazol-5(4H)-one as yellow gum (350 mg, Yield: Crude). TLC system: MeOH/DCM (10:90), R_{f} value: ˜0.3; LCMS (m/z): 497.27 (M+H)⁺.

### Synthesis of 3-(6-fluoro-5-(4-(1-hydroxycyclobutyl)phenyl)-1H-pyrrolo[3,2-b]pyridin-3-yl)-1,2,4-oxadiazol-5(4H)-one

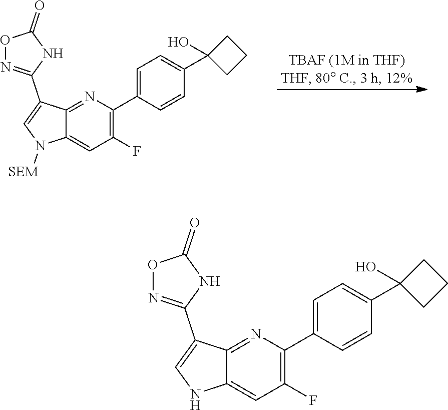 [SMILES: C[Si](C)(C)CCOCn1cc(-c2noc(=O)[nH]2)c2nc(-c3ccc(C4(O)CCC4)cc3)c(F)cc21 ; O=c1[nH]c(-c2c[nH]c3cc(F)c(-c4ccc(C5(O)CCC5)cc4)nc23)no1]

To a stirred solution of 3-(6-fluoro-5-(4-(1-hydroxycyclobutyl)phenyl)-1-((2-(trimethylsilyl)ethoxy)methyl)-1H-pyrrolo[3,2-b]pyridin-3-yl)-1,2,4-oxadiazol-5(4H)-one (350 mg, 0.70 mmol, 1.0 eq) in THF (3.5 mL) at 0° C., was added TBAF (1.0 M in THF) (7 mL, 7.00 mmol, 10 eq) and stirred at 80° C. for 3 h. After completion of reaction by TLC, diluted with water (50 mL) and extracted with 10% MeOH in DCM (2×20 mL). The combined organic layer was washed with NH₄Cl solution (30 mL), dried over Na₂SO₄, filtered and concentrated under reduced pressure. The crude product was purified by reverse phase column chromatography [elution with 50% ACN in 0.1% FA in H₂O] to afford product with TBAF impurities. Re-purified by prep-HPLC and pure fractions were lyophilized to afford 3-(6-fluoro-5-(4-(1-hydroxycyclobutyl)phenyl)-1H-pyrrolo[3,2-b]pyridin-3-yl)-1,2,4-oxadiazol-5(4H)-one as off white solid (31 mg, Yield: 12%). TLC system: MeOH/DCM (10:90), R_{f} value: ˜0.2; LCMS (m/z): 367.1 (M+H)⁺; ¹H NMR (400 MHz, DMSO-d₆) δ: 12.46 (bs, 1H), 12.23 (s, 1H), 8.25 (s, 1H), 7.98-7.93 (m, 3H), 7.64 (d, J=8.4 Hz, 2H), 5.59 (s, 1H), 2.51-2.41 (m, 2H), 2.36-2.28 (m, 2H), 1.99-1.93 (m, 1H), 1.73-1.67 (m, 1H).

Prep conditions: C0615-85-P1; Obtained Sample quantity 90 mg; Column GEMINI C18 (250*21.2) mm 5mic; Buffer MP-(A): 0.1% TFA IN WATER; MP-(B): Acetonitrile; Gradient (T/% B); 0/30, 15/60, 16/60, 16.50/95, 19/95, 19.50/30, 23/30; Flow Rate: 13 ml/min; Solubility WATER:THF:ACN

### SYNTHETIC EXAMPLE 36

### Synthesis of Compound I-45

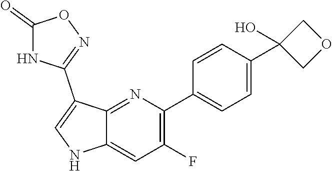 [SMILES: O=c1[nH]c(-c2c[nH]c3cc(F)c(-c4ccc(C5(O)COC5)cc4)nc23)no1]

### Synthesis of 3-(4-(4,4,5,5-tetramethyl-1,3,2-dioxaborolan-2-yl)phenyl)oxetan-3-ol

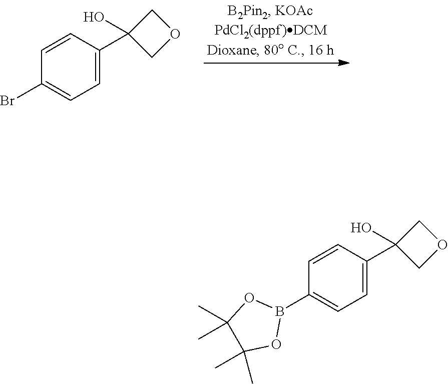 [SMILES: OC1(c2ccc(Br)cc2)COC1 ; CC1(C)OB(c2ccc(C3(O)COC3)cc2)OC1(C)C]

In a sealed tube to a degassed solution of 3-(4-bromophenyl)oxetan-3-ol (1 g, 4.37 mmol, 1.0 eq) and B₂Pin₂ (1.66 g, 6.55 mmol, 1.5 eq) in Dioxane (20 Vol, 20 mL) at room temperature, was added KOAc (1.28 g, 13.1 mmol, 3.0 eq) followed by Pd(dppf)Cl₂·DCM (357 mg, 0.43 mmol, 0.1 eq) and stirred at 80° C. for 16 h. After completion of reaction by TLC, reaction mixture was diluted with water (20 mL) and extracted with EtOAc (2×20 mL). The combined organic layer was dried over Na₂SO₄, filtered and concentrated under reduced pressure to afford 3-(4-(4,4,5,5-tetramethyl-1,3,2-dioxaborolan-2-yl)phenyl)oxetan-3-ol as brown solid (1.2 g, Crude). TLC system: EtOAc/Hexane (30:70), R_{f} value: ˜0.4; ¹H NMR (400 MHz, CDCl₃) δ: 7.87 (d, J=8.4 Hz, 2H), 7.61 (d, J=8.4 Hz, 2H), 4.92 (s, 4H), 1.35 (s, 12H).

### Synthesis of 6-fluoro-5-(4-(3-hydroxyoxetan-3-yl)phenyl)-1-((2-(trimethylsilyl)ethoxy)methyl)-1H-pyrrolo[3,2-b]pyridine-3-carbonitrile

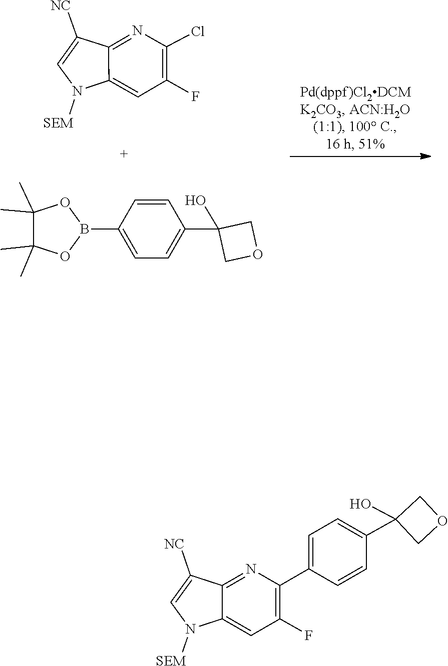 [SMILES: C[Si](C)(C)CCOCn1cc(C#N)c2nc(Cl)c(F)cc21 ; CC1(C)OB(c2ccc(C3(O)COC3)cc2)OC1(C)C ; C[Si](C)(C)CCOCn1cc(C#N)c2nc(-c3ccc(C4(O)COC4)cc3)c(F)cc21]

In a sealed tube, to a degassed solution of 5-chloro-6-fluoro-1-((2-(trimethylsilyl)ethoxy)methyl)-1H-pyrrolo[3,2-b]pyridine-3-carbonitrile (350 mg, 1.07 mmol, 1.0 eq) and 3-(4-(4,4,5,5-tetramethyl-1,3,2-dioxaborolan-2-yl)phenyl)oxetan-3-ol (443 mg, 1.6 mmol, 1.5 eq) in ACN:H₂O (1:1, 3.5 mL) at room temperature, was added K₂CO₃ (443 mg, 3.21 mmol, 3.0 eq) Pd(dppf)Cl₂·DCM (88 mg, 0.11 mmol, 0.1 eq) and stirred at 100° C. for 16 h. After completion of reaction by TLC, the reaction mixture was diluted with water (20 mL) and extracted with EtOAc (2×20 mL). The combined organic layer was dried over Na₂SO₄, filtered and concentrated under reduced pressure to afford crude product. The crude product was purified by silica gel [100-200] column chromatography [elution with 30% EtOAc in petroleum ether] to afford 6-fluoro-5-(4-(3-hydroxyoxetan-3-yl)phenyl)-1-((2-(trimethylsilyl)ethoxy)methyl)-1H-pyrrolo[3,2-b]pyridine-3-carbonitrile as an off white solid (400 mg, Yield: 51%). TLC system: EtOAc/Hexane (50:50), R_{f} value: ˜0.3; LCMS (m/z): 440.22 (M+H)⁺; ¹H NMR (400 MHz, CDCl₃) δ: 8.15 (d, J=8.0 Hz, 2H), 7.91 (s, 1H), 7.77 (d, J=8.0 Hz, 2H), 7.71 ((d, J=10.8 Hz, 1H), 5.52 (s, 2H), 4.99 (s, 4H), 3.54 (t, J=8.0 Hz, 2H), 0.95 (t, J=8.4 Hz, 2H), 0.02 (s, 9H).

### Synthesis of (Z)-6-fluoro-N′-hydroxy-5-(4-(3-hydroxyoxetan-3-yl)phenyl)-1-((2-(trimethylsilyl)ethoxy)methyl)-1H-pyrrolo[3,2-b]pyridine-3-carboximidamide

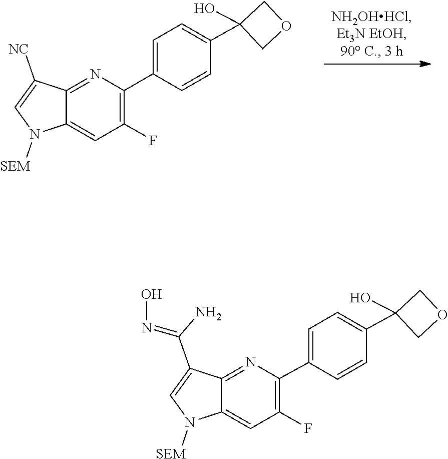 [SMILES: C[Si](C)(C)CCOCn1cc(C#N)c2nc(-c3ccc(C4(O)COC4)cc3)c(F)cc21 ; C[Si](C)(C)CCOCn1cc(/C(N)=N/O)c2nc(-c3ccc(C4(O)COC4)cc3)c(F)cc21]

To a stirred solution of 6-fluoro-5-(4-(3-hydroxyoxetan-3-yl)phenyl)-1-((2-(trimethylsilyl)ethoxy)methyl)-1H-pyrrolo[3,2-b]pyridine-3-carbonitrile (400 mg, 0.91 mmol, 1.0 eq) in EtOH (4 mL) at room temperature, was added Et₃N (276 mg, 2.73 mmol, 3.0 eq), NH₂OH·HCl (75 mg, 1.09 mmol, 1.2 eq) and stirred at 90° C. for 3 h. After completion of reaction by TLC, the reaction mixture was diluted with water (20 mL) and extracted with 10% MeOH in DCM (2×20 mL). The combined organic layer was dried over Na₂SO₄, filtered and concentrated under reduced pressure to afford (Z)-6-fluoro-N′-hydroxy-5-(4-(3-hydroxyoxetan-3-yl)phenyl)-1-((2-(trimethylsilyl)ethoxy)methyl)-1H-pyrrolo[3,2-b]pyridine-3-carboximidamide as a yellow gum (400 mg, Yield: Crude). TLC system: MeOH/DCM (10:90), R_{f} value: ˜0.2; LCMS (m/z): 473.38 (M+H)⁺.

### Synthesis of ethyl (Z)-((6-fluoro-5-(4-(3-hydroxyoxetan-3-yl)phenyl)-1-((2-(trimethylsilyl)ethoxy)methyl)-1H-pyrrolo[3,2-b]pyridin-3-yl) (hydroxyimino)methyl)carbamate

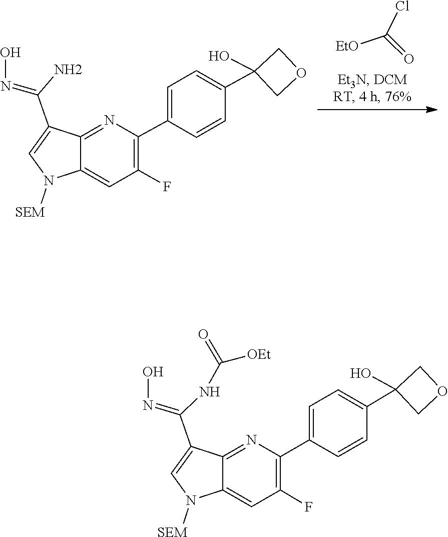 [SMILES: C[Si](C)(C)CCOCn1cc(/C(N)=N/O)c2nc(-c3ccc(C4(O)COC4)cc3)c(F)cc21 ; CCOC(=O)Cl ; CCOC(=O)N/C(=N\O)c1cn(COCC[Si](C)(C)C)c2cc(F)c(-c3ccc(C4(O)COC4)cc3)nc12]

To a stirred solution of (Z)-6-fluoro-N′-hydroxy-5-(4-(3-hydroxyoxetan-3-yl)phenyl)-1-((2-(trimethylsilyl)ethoxy)methyl)-1H-pyrrolo[3,2-b]pyridine-3-carboximidamide (400 mg, 0.85 mmol, 1.0 eq) in DCM (4 mL) at 0° C., was added Et₃N (112 mg, 1.1 mmol, 1.3 eq) and ethylchloroformate (110 mg, 1.02 mmol, 1.2 eq). The reaction mixture was stirred at room temperature for 4 h. After completion of reaction by TLC, the reaction mixture was quenched with water (20 mL) and extracted with DCM (2×20 mL). The combined organic layer was dried over Na₂SO₄, filtered and concentrated to afford crude product. The crude product was purified by silica gel [100-200] column chromatography [elution with 30% EtOAc in petroleum ether] to afford ethyl (Z)-((6-fluoro-5-(4-(3-hydroxyoxetan-3-yl)phenyl)-1-((2-(trimethylsilyl)ethoxy)methyl)-1H-pyrrolo[3,2-b]pyridin-3-yl)(hydroxyimino)methyl)carbamate as yellow gum (350 mg, Yield: 76%). TLC system: EtOAc/Hexane (50:50), R_{f} value: ˜0.5; LCMS (m/z): 545.39 (M+H)⁺; ¹H NMR (400 MHz, CDCl₃) δ: 8.02-8.00 (m, 2H), 7.78-7.76 (m, 2H), 7.73-7.70 (m, 1H), 5.49 (s, 2H), 5.00-4.96 (m, 4H), 4.37 (q, J=7.2 Hz, 2H), 3.53-3.49 (m, 2H), 1.41-1.37 (m, 3H), 0.94-0.86 (m, 2H), −0.02 (s, 9H).

### Synthesis of 3-(6-fluoro-5-(4-(3-hydroxyoxetan-3-yl)phenyl)-1-((2-(trimethylsilyl)ethoxy)methyl)-1H-pyrrolo[3,2-b]pyridin-3-yl)-1,2,4-oxadiazol-5(4H)-one

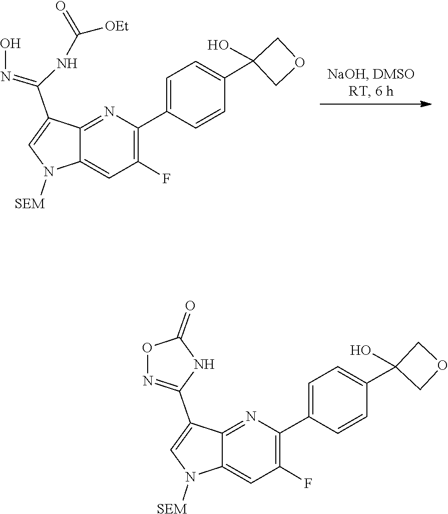 [SMILES: CCOC(=O)N/C(=N\O)c1cn(COCC[Si](C)(C)C)c2cc(F)c(-c3ccc(C4(O)COC4)cc3)nc12 ; C[Si](C)(C)CCOCn1cc(-c2noc(=O)[nH]2)c2nc(-c3ccc(C4(O)COC4)cc3)c(F)cc21]

To a stirred solution of ethyl (Z)-((6-fluoro-5-(4-(3-hydroxyoxetan-3-yl)phenyl)-1-((2-(trimethylsilyl)ethoxy)methyl)-1H-pyrrolo[3,2-b]pyridin-3-yl)(hydroxyimino)methyl)carbamate (350 mg, 0.64 mmol, 1.0 eq) in DMSO (1.3 mL) at room temperature, was added NaOH (31 mg, 0.77 mmol, 1.2 eq) and reaction mixture was stirred at room temperature for 6 h. After completion of reaction by TLC, the reaction mixture was quenched with 2N aq. HCl solution (10 mL), precipitation observed. Solid filtered, washed with n-pentane (10 mL) and dried under vacuum to afford 3-(6-fluoro-5-(4-(3-hydroxyoxetan-3-yl)phenyl)-1-((2-(trimethylsilyl)ethoxy)methyl)-1H-pyrrolo[3,2-b]pyridin-3-yl)-1,2,4-oxadiazol-5(4H)-one as yellow gum (300 mg, Crude). TLC system: MeOH/DCM (5:95), R_{f} value: ˜0.2; LCMS (m/z): 499.3 (M+H)⁺.

### Synthesis of 3-(6-fluoro-5-(4-(3-hydroxyoxetan-3-yl)phenyl)-1H-pyrrolo[3,2-b]pyridin-3-yl)-1,2,4-oxadiazol-5(4H)-one

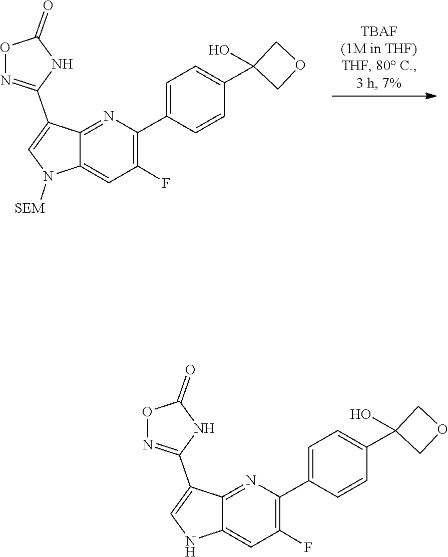 [SMILES: C[Si](C)(C)CCOCn1cc(-c2noc(=O)[nH]2)c2nc(-c3ccc(C4(O)COC4)cc3)c(F)cc21 ; O=c1[nH]c(-c2c[nH]c3cc(F)c(-c4ccc(C5(O)COC5)cc4)nc23)no1]

To a stirred solution of 3-(6-fluoro-5-(4-(3-hydroxyoxetan-3-yl)phenyl)-1-((2-(trimethylsilyl)ethoxy)methyl)-1H-pyrrolo[3,2-b]pyridin-3-yl)-1,2,4-oxadiazol-5(4H)-one (300 mg, 0.6 mmol, 1.0 eq) in THF (3.0 mL) at 0° C., was added TBAF (1.0 M in THF) (6.0 mL, 6.02 mmol, 10 eq) and stirred at 80° C. for 3 h. After completion of reaction by TLC, the reaction mixture was diluted with water (30 mL) and extracted with 10% MeOH in DCM (2×30 mL). The combined organic layer was dried over Na₂SO₄, filtered, concentrated and purified by Prep-HPLC. Collected fractions were frozen and lyophilized to afford 3-(6-fluoro-5-(4-(3-hydroxyoxetan-3-yl)phenyl)-1H-pyrrolo[3,2-b]pyridin-3-yl)-1,2,4-oxadiazol-5(4H)-one as white solid (15 mg, Yield: 7%). TLC system: MeOH/DCM (10:90), R_{f} value: ˜0.2; LCMS (m/z): 369.32 (M+H)⁺; ¹H NMR (400 MHz, DMSO-d₆) δ: 12.16 (s, 1H), 8.23 (s, 1H), 8.02-7.95 (m, 3H), 7.77-7.75 (m, 2H), 6.45 (s, 1H), 4.82 (d, J=6.4 Hz, 2H), 4.75 (d, J=6.4 Hz, 2H).

Prep-HPLC condition: Column X-Bridge, C18, (250*19.0 mm) 5u, Buffer A: 0.1% TFA IN WATER; B: A CN:H2O (80:20) Gradient (T/% B) 0/35, 15/45, 25/95/98@13ML/MIN Flow Rate 13 ml/min Solubility ACN+H2O

### SYNTHETIC EXAMPLE 37

### Synthesis of Compound I-57

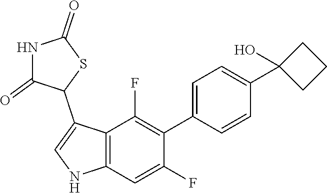 [SMILES: O=C1NC(=O)C(c2c[nH]c3cc(F)c(-c4ccc(C5(O)CCC5)cc4)c(F)c23)S1]

### Synthesis of 5-bromothiazolidine-2,4-dione

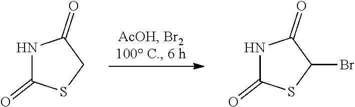 [SMILES: O=C1CSC(=O)N1 ; O=C1NC(=O)C(Br)S1]

To a stirred solution of thiazolidine-2,4-dione (3 g, 25.6 mmol, 1.0 eq) in acetic acid (4.5 mL, 1.5 Vol), was added Br₂ (1.3 mL, 25.6 mmol, 1.0 eq) in acetic acid (0.7, 0.25 Vol) at 100° C., continued stirring at 100° C. for 6 h. After completion of reaction by TLC, diluted with water and extracted with DCM (2×50 mL). Organic layer was separated, washed with brine solution, dried over sodium sulfate and concentrated. Obtained crude compound was purified by silica (100-200 mesh) column chromatography [eluted with 50% EtOAc/Hexane] and pure fractions were concentrated under reduced pressure to afford 5-bromothiazolidine-2,4-dione as a gummy liquid (3 g, yield: 61%). TLC system: EtOAC/Hexane (50:50) R_{f} value: ˜0.5 (KMnO4 stain); ¹H NMR (400 MHz, CDCl₃) δ 8.97 (br s, 1H), 5.89 (s, 1H).

### Synthesis of 5-(5-bromo-4,6-difluoro-1H-indol-3-yl) thiazolidine-2,4-dione

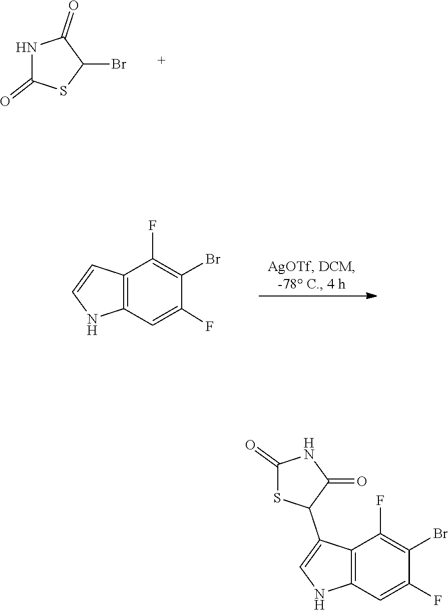 [SMILES: O=C1NC(=O)C(Br)S1 ; Fc1cc2[nH]ccc2c(F)c1Br ; O=C1NC(=O)C(c2c[nH]c3cc(F)c(Br)c(F)c23)S1]

To a stirred and solution of AgOTf (1.10 g, 4.3 mmol, 2.0 eq) in DCM (5 mL, 10 Vol) at −78° C. was added 5-bromo-4,6-difluoro-1H-indole (500 mg, 2.2 mmol, 1.0 eq) followed by 5-bromothiazolidine-2,4-dione (1.25 g, 6.5 mmol, 3.0 eq) slowly, and reaction mixture was stirred at −78° C. for 4 h. After completion of reaction by TLC, the reaction mixture was diluted with water and extracted with DCM (2×50 mL). Organic layer was separated, washed with brine solution, dried over sodium sulfate and concentrated. Obtained crude was purified by reverse phase C18 column chromatography [eluted with 45% ACN with 0.1% FA in H₂O], pure fractions was concentrated under reduced pressure to afford 5-(5-bromo-4,6-difluoro-1H-indol-3-yl) thiazolidine-2,4-dione as white solid (180 mg, yield: 24%). TLC system: EtOAC/Hexane (50:50) R_{f} value: ^{˜}0.3; LCMS (m/z): 344.98 (M−H)⁺; ¹H NMR (400 MHz, DMSO-d₆) δ 12.24 (bs, 1H), 11.76 (s, 1H), 7.59 (d, J=2.4 Hz, 1H), 7.32 (dd, J=8.0 & 0.8 Hz, 1H), 6.14 (s, 1H).

### Synthesis of 5-(4,6-difluoro-5-(4-(1-hydroxycyclobutyl)phenyl)-1H-indol-3-yl) thiazolidine-2,4-dione

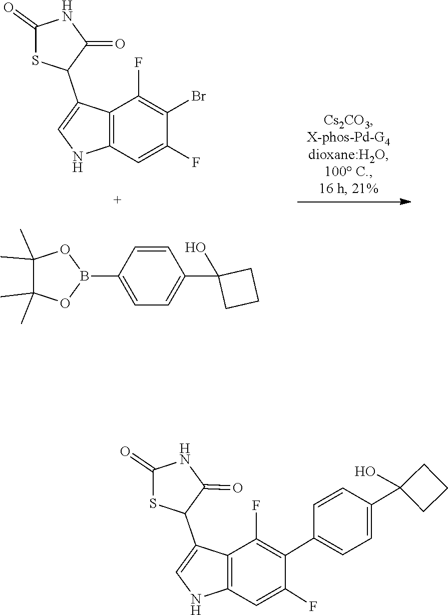 [SMILES: O=C1NC(=O)C(c2c[nH]c3cc(F)c(Br)c(F)c23)S1 ; CC1(C)OB(c2ccc(C3(O)CCC3)cc2)OC1(C)C ; O=C1NC(=O)C(c2c[nH]c3cc(F)c(-c4ccc(C5(O)CCC5)cc4)c(F)c23)S1]

To a stirred solution of 5-(5-bromo-4,6-difluoro-1H-indol-3-yl) thiazolidine-2,4-dione (180 mg, 0.52 mmol, 1 eq) in Dioxane: Water (2:1) (5.5 mL), was added Cs₂CO₃ (507 mg, 1.56 mmol, 3 eq) followed by 1-(4-(4,4,5,5-tetramethyl-1,3,2-dioxaborolan-2-yl)phenyl)cyclobutan-1-ol (356 mg, 1.30 mmol, 2.5 eq) and degassed for 10 min, later added X-phos-Pd-G₄ (45 mg, 0.05 mmol, 0.1 eq). RM was degassed for 10 min and the reaction mixture was stirred at 100° C. for 16 h. After completion of reaction by TLC, reaction mixture filtered through a pad of diatomaceous earth (e.g., Celite®), and washed with ethyl acetate. Organic layer was separated, washed with brine solution, dried over sodium sulfate and concentrated. Obtained crude compound was purified by Prep-HPLC and pure fractions were lyophilized to afford 5-(4,6-difluoro-5-(4-(1-hydroxycyclobutyl)phenyl)-1H-indol-3-yl) thiazolidine-2,4-dione as white solid (46 mg, yield: 21%). TLC system: EtOAC/Hexane (50:50) R_{f} value: ^{˜}0.1; LCMS (m/z): 413.19 (M+H)⁺; ¹H NMR (400 MHz, DMSO-d₆) δ 12.11 (s, 1H), 11.63 (s, 1H), 7.60-7.57 (m, 3H), 7.35 (d, J=8.4 Hz, 2H), 7.22 (d, J=10 Hz, 1H), 6.18 (s, 1H), 5.53 (s, 1H), 2.49-2.41 (m, 2H), 2.33-2.26 (m, 2H), 1.98-1.88 (m, 1H), 1.74-1.65 (m, 1H).

Prep Conditions: Column GEMINI C18 (250*21.2) mm 5mic Buffer MP-(A): 0.1% FA IN WATER MP-(B): Acetonitrile:WATER (80:20), Gradient (T/% B) 0/50, 15/70, 25/98, 30/98, Flow Rate 13 ml/min, Solubility WATER, Acetonitrile, THF

### SYNTHETIC EXAMPLE 38

### Synthesis of Compound I-61

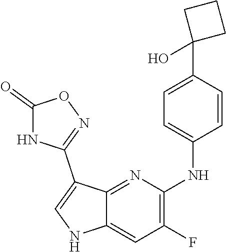 [SMILES: O=c1[nH]c(-c2c[nH]c3cc(F)c(Nc4ccc(C5(O)CCC5)cc4)nc23)no1]

### Synthesis of N-(4-bromophenyl)-2,2,2-trifluoroacetamide

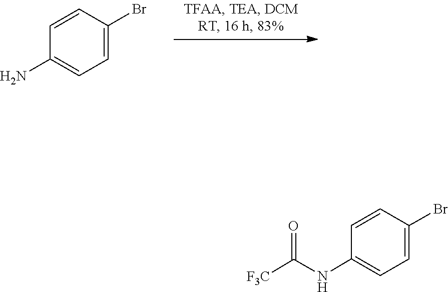 [SMILES: Nc1ccc(Br)cc1 ; O=C(Nc1ccc(Br)cc1)C(F)(F)F]

To a stirred solution of 4-bromoaniline (10 g, 29.1 mmol, 1 eq), in DCM (100 mL), at 0° C., was added Et₃N (1.2 mL, 46.5 mmol, 1.6 eq), TFAA (11.2 mL, 84.3 mmol, 2.9 eq) and stirred for 16 h at room temperature. After completion of reaction by TLC, the reaction mixture was quenched with 1 N aq. NaOH solution (100 mL) and extracted with DCM (2×100 mL). The combined organic layer was dried over Na₂SO₄, filtered and concentrated under reduced pressure. The crude was purified by silica gel (60-120 mesh) column chromatography [eluted with 5-10% EtOAc in hexane] to afford N-(4-bromophenyl)-2,2,2-trifluoroacetamide as white solid (11 g, 83% yield), TLC system: EtOAc: hexane (20:80), R_{f} value: ˜0.5; LCMS (m/z): 265.97 (M−H).⁻¹H NMR (400 MHz, CDCl₃). δ: 7.97 (brs, 1H), 7.54-7.46 (m, 4H).

### Synthesis of N-(4-bromophenyl)-2,2,2-trifluoroacetamide

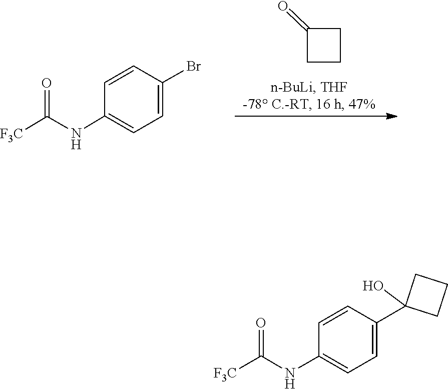 [SMILES: O=C1CCC1 ; O=C(Nc1ccc(Br)cc1)C(F)(F)F ; O=C(Nc1ccc(C2(O)CCC2)cc1)C(F)(F)F]

To a stirred solution of N-(4-bromophenyl)-2,2,2-trifluoroacetamide (5 g, 18.6 mmol, 1 eq), in THF (100 mL), cooled to −78° C., added n-BuLi 1.6 M in THF (44.3 mL, 70.9 mmol, 3.8 eq) and stirred for 30 min. Later at same temperature, added cyclobutanone (1.95 g, 27.9 mmol, 1.5 eq) and allowed to room temperature, stirred for 16 h. After completion of reaction by TLC, quenched with NH₄Cl solution (100 mL) and extracted with EtOAc (2×100 mL). Organic layer was dried over Na₂SO₄, filtered and concentrated under reduced pressure. Obtained crude was purified by silica gel (100-200 mesh) column chromatography [eluted with 20% EtOAc in hexane] to afford 2,2,2-trifluoro-N-(4-(1-hydroxycyclobutyl)phenyl)acetamide as off white solid (2.3 g, 47% yield), TLC system: EtOAc: hexane (30:70), R_{f} value: ˜0.3. LCMS (m/z): 258.16 (M−H)⁺.

### Synthesis of 1-(4-aminophenyl)cyclobutan-1-ol

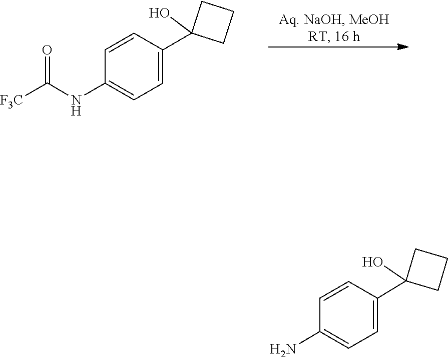 [SMILES: O=C(Nc1ccc(C2(O)CCC2)cc1)C(F)(F)F ; Nc1ccc(C2(O)CCC2)cc1]

To a stirred solution of 2,2,2-trifluoro-N-(4-(1-hydroxycyclobutyl)phenyl)acetamide 10.0 mmol, 1 eq), in MeOH (23 mL) at room temperature was added 1 N aq. NaOH (46 mL, 20 vol) and continued for 16 h. After completion of reaction by TLC, quenched with water (50 mL) and extracted with DCM (2×50 mL). Combined organic layer was dried over Na₂SO₄, filtered and concentrated under reduced pressure to afford 1-(4-aminophenyl)cyclobutan-1-ol as brown gummy solid (1.5 g, crude), TLC system: EtOAc: hexane (50:50), R_{f} value: ˜0.4. LCMS (m/z): 164.11 (M+H)⁺. ¹H NMR (400 MHz, CDCl₃) δ: 7.30 (dd, J=2 & 6.4 Hz, 2H), 6.70 (dd, J=2 & 6.4 Hz, 2H), 2.54-2.50 (m, 2H), 2.37-2.30 (m, 2H), 1.98-1.91 (m, 1H), 1.67-1.57 (m, 1H).

### Synthesis of 6-fluoro-5-((4-(1-hydroxycyclobutyl)phenyl)amino)-1-((2-(trimethylsilyl)ethoxy)methyl)-1H-pyrrolo[3,2-b]pyridine-3-carbonitrile

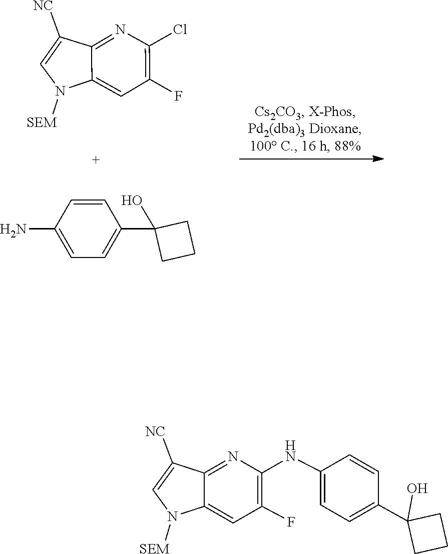 [SMILES: C[Si](C)(C)CCOCn1cc(C#N)c2nc(Cl)c(F)cc21 ; Nc1ccc(C2(O)CCC2)cc1 ; C[Si](C)(C)CCOCn1cc(C#N)c2nc(Nc3ccc(C4(O)CCC4)cc3)c(F)cc21]

To a stirred solution off 5-chloro-6-fluoro-1-((2-(trimethylsilyl)ethoxy)methyl)-1H-pyrrolo[3,2-b]pyridine-3-carbonitrile (900 mg, 2.77 mmol, 1 eq), in 1.4 dioxane (10 mL), was added 1-(4-aminophenyl)cyclobutan-1-ol (681 mg, 4.15 mmol, 1.5 eq), Cs₂CO₃ (1.80 g, 5.54 mmol, 2 eq) at room temperature, degassed with nitrogen for 5 min. Later added X-phos (527 mg, 1.10 mmol, 0.4 eq), Pd₂(dba)₃ (180 mg, 0.55 mmol, 0.2 eq) and degassed with nitrogen for additional 10 min, heated to 100° C., stirred for 16 h. After completion of reaction by TLC, quenched with water (25 mL) and extracted with EtOAc (2×50 mL). Combined organic layer was dried over Na₂SO₄, filtered, concentrated under reduced pressure and purified by silica gel (100-200 mesh) column chromatography [eluted with 40% EtOAc in hexane] to afford 6-fluoro-5-((4-(1-hydroxycyclobutyl)phenyl)amino)-1-((2-(trimethylsilyl)ethoxy)methyl)-1H-pyrrolo[3,2-b]pyridine-3-carbonitrile as off orange gummy (1.1 g, Yield: 88%). TLC system: EtOAc: hexane (50:50), R_{f} value: ˜0.3; LCMS (m/z): 453.2 (M+H)⁺.

### Synthesis of ((Z)-6-fluoro-N′-hydroxy-5-((4-(1-hydroxycyclobutyl)phenyl)amino)-1-((2-(trimethylsilyl)ethoxy)methyl)-1H-pyrrolo[3,2-b]pyridine-3-carboximidamide

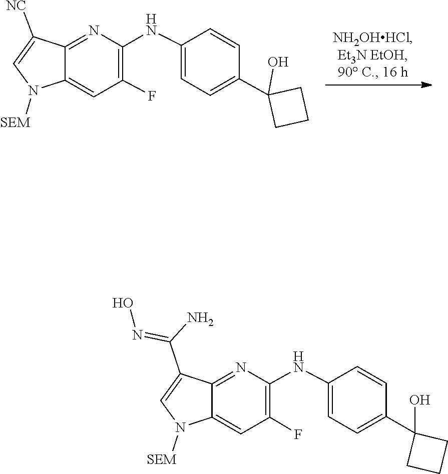

To a stirred solution off 6-fluoro-5-((4-(1-hydroxycyclobutyl)phenyl)amino)-1-((2-(trimethylsilyl)ethoxy)methyl)-1H-pyrrolo[3,2-b]pyridine-3-carbonitrile (1.1 g, 2.43 mmol, 2 eq) in EtOH (11 mL) at room temperature was added Et₃N (491 mg, 4.86 mmol, 2 eq), NH₂OH. HCl (335 mg, 4.86 mmol, 2 eq) and stirred at 90° C. for 16 h. After completion of reaction by TLC, diluted with ice water (20 mL) and extracted with 10% MeOH:DCM (2×50 mL). Combined organic layer was dried over Na₂SO₄, filtered and concentrated under reduced pressure to afford (Z)-6-fluoro-N′-hydroxy-5-((4-(1-hydroxycyclobutyl)phenyl)amino)-1-((2-(trimethylsilyl)ethoxy)methyl)-1H-pyrrolo[3,2-b]pyridine-3-carboximidamide as black gummy (1.1 g, crude), TLC system: EtOAc, R_{f} value: ˜0.1; LCMS (m/z): 486.3 (M+H)⁺.

### Synthesis of 3-(6-fluoro-5-((4-(1-hydroxycyclobutyl)phenyl)amino)-1-((2-(trimethylsilyl)ethoxy)methyl)-1H-pyrrolo[3,2-b]pyridin-3-yl)-1,2,4-oxadiazol-5(4H)-one

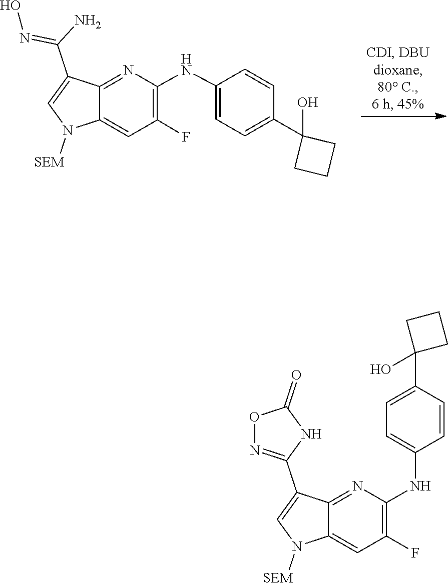

To a stirred solution of (Z)-6-fluoro-N′-hydroxy-5-((4-(1-hydroxycyclobutyl)phenyl)amino)-1-((2-(trimethylsilyl)ethoxy)methyl)-1H-pyrrolo[3,2-b]pyridine-3-carboximidamide (1.1 g, 2.26 mmol, 1 eq) in 1,4 dioxane (11 mL) at room temperature was added CDI (734 mg, 4.53 mmol, 2 eq), DBU (1.37 g, 9.07 mmol, 4 eq) and heated at 80° C. for 16 h. After completion of reaction by TLC, diluted with ice cold water (30 mL) and extracted with 10% MeOH/DCM (3×50 mL). Combined organic layer was dried over Na₂SO₄, filtered and concentrated under reduced pressure. Obtained crude was purified by reverse phase C18 column chromatography [eluted with 70% ACN with 0.1% FA in H₂O] and pure fractions were concentrated to afford 3-(6-fluoro-5-((4-(1-hydroxycyclobutyl)phenyl)amino)-1-((2-(trimethylsilyl)ethoxy)methyl)-1H-pyrrolo[3,2-b]pyridin-3-yl)-1,2,4-oxadiazol-5(4H)-one (500 mg, Yield: 45%). TLC system: 10% MeOH/DCM (10:90), R_{f} value: ˜0.2; LCMS (m/z): 512.32 (M+H)⁺;

### Synthesis of 3-(6-fluoro-5-((4-(1-hydroxycyclobutyl)phenyl)amino)-1H-pyrrolo[3,2-b]pyridin-3-yl)-1,2,4-oxadiazol-5(4H)-one

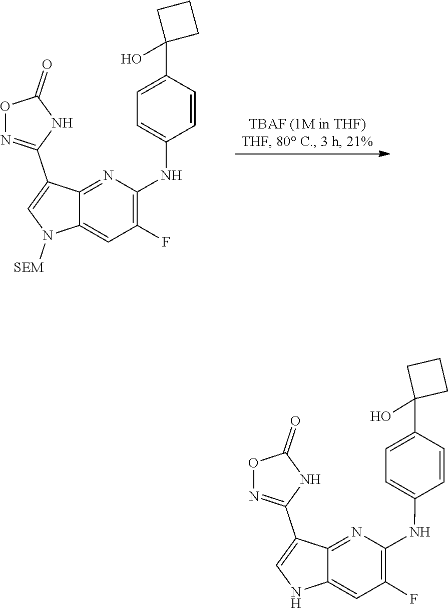 [SMILES: C[Si](C)(C)CCOCn1cc(-c2noc(=O)[nH]2)c2nc(Nc3ccc(C4(O)CCC4)cc3)c(F)cc21 ; O=c1[nH]c(-c2c[nH]c3cc(F)c(Nc4ccc(C5(O)CCC5)cc4)nc23)no1]

To a stirred solution of 3-(6-fluoro-5-((4-(1-hydroxycyclobutyl)phenyl)amino)-1-((2-(trimethylsilyl)ethoxy)methyl)-1H-pyrrolo[3,2-b]pyridin-3-yl)-1,2,4-oxadiazol-5(4H)-one (500 mg, 0.97 mmol, 1 eq), in THF (5 mL) at room temperature was added TBAF (1.0M) (9.7 mL, 9.78 mmol, 10 eq) and stirred for 3 h at 80° C. After completion of reaction by TLC, quenched with saturated NH₄Cl solution (20 mL) and extracted with 10% MeOH/DCM (2×50 mL). Combined organic layer was dried over Na₂SO₄, filtered and concentrated under reduced pressure. Obtained crude was purified by reverse phase C18 column chromatography [eluted with 50% ACN with 0.1% FA in H₂O], followed by PREP-HPLC (ABC method) purification afforded 3-(6-fluoro-5-((4-(1-hydroxycyclobutyl)phenyl)amino)-1H-pyrrolo[3,2-b]pyridin-3-yl)-1,2,4-oxadiazol-5(4H)-one as off white solid (80 mg, Yield: 21%). TLC system: MeOH/DCM (10:90), R_{f} value: ˜0.2; LCMS (m/z): 380.19 (M−H)⁻. ¹H NMR (400 MHz, DMSO-d₆) δ: 11.55 (brs, 1H), 8.66 (d, J=2.0 Hz, 1H), 8.09 (d, J=8.8 Hz, 2H), 7.91 (s, 1H), 7.77 (d, J=12 Hz, 1H), 7.34 (d, J=8.4 Hz, 2H), 5.33 (s, 1H), 2.42-2.40 (m, 2H), 2.39-2.20 (m, 2H), 1.94-1.84 (m, 1H), 1.66-1.57 (m, 1H).

Prep Conditions: Sample code: C0827-4-P; Column: GEMINI NX C₁₈ (250*21.2) mm 5mic; Buffer: MP-(A): 10 Mm ABC IN WATER MP-(B): Acetonitrile:WATER (80:20); Gradient: (T/% B) 0/20, 15/60, 25/95; Flow Rate: 14 ml/min; Solubility: WATER:ACN:THF.

### SYNTHETIC EXAMPLE 39

### Synthesis of Compound I-62

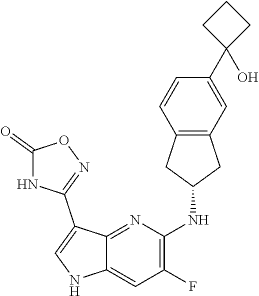 [SMILES: O=c1[nH]c(-c2c[nH]c3cc(F)c(N[C@@H]4Cc5ccc(C6(O)CCC6)cc5C4)nc23)no1]

### Synthesis of 5-bromo-2,3-dihydro-1H-inden-2-ol

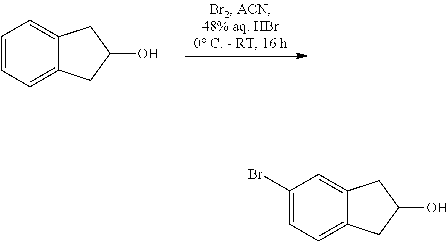 [SMILES: OC1Cc2ccccc2C1 ; OC1Cc2ccc(Br)cc2C1]

To a stirred solution of 2,3-dihydro-1H-inden-2-ol (5 g, 37.3 mmol, 1.0 eq) in ACN (30 mL, 6 Vol) at 0° C., was added 48% aq. HBr (150 mL, 30 Vol) followed by Br₂ (11.9 g, 74.6 mmol, 2.0 eq) drop wise. The reaction mixture was stirred at room temperature for 16 h. After completion of reaction by TLC, the reaction mixture was quenched with Na₂CO₃ solution (30 mL) (adjust PH=8) and extracted with EtOAc (2×30 mL). The combined organic layer was dried over Na₂SO₄, filtered and concentrated under reduced pressure to crude product. The crude product was triturated with n-pentane (30 mL), decanted and dried under vacuum to afford 5-bromo-2,3-dihydro-1H-inden-2-ol as white solid (6 g, Yield: Crude). TLC system: EtOAc/Hexane (20:80), R_{f} value: ˜0.4; ¹H NMR (400 MHz, CDCl₃) δ: 7.37 (s, 1H), 7.30-7.28 (m, 1H), 7.10 (d, J=8.0 Hz, 1H), 4.72-4.67 (m, 1H), 3.26-3.11 (m, 2H), 2.98-2.01 (m, 2H).

### Synthesis of 5-bromo-2,3-dihydro-1H-inden-2-yl methanesulfonate

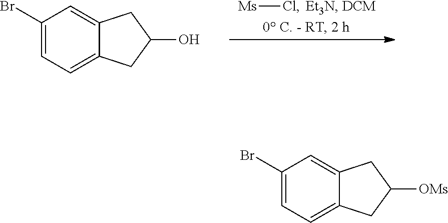

To a stirred solution of 5-bromo-2,3-dihydro-1H-inden-2-ol (6 g, 28.2 mmol, 1.0 eq) in DCM (60 mL) at 0° C., was added Et₃N (5.6 g, 56.4 mmol, 2.0 eq), Ms-Cl (3.9 g, 33.8 mmol, 1.2 eq) and stirred at room temperature for 2 h. After completion of reaction by TLC, the reaction mixture was quenched with water (40 mL) and extracted with DCM (2×40 mL). The combined organic layer was washed with NaHCO₃ solution (50 mL), dried over Na₂SO₄, filtered and concentrated under reduced pressure to afford 5-bromo-2,3-dihydro-1H-inden-2-yl methanesulfonate as yellow solid (8 g, Crude). TLC system: EtOAc/Hexane (20:80), R_{f} value: ˜0.5; ¹H NMR (400 MHz, CDCl₃) δ: 7.39 (s, 1H), 7.35-7.33 (m, 1H), 7.12 (d, J=8.0 Hz, 1H), 5.54-5.49 (m, 1H), 3.38-3.21 (m, 4H), 3.02 (s, 3H).

### Synthesis of 2-azido-5-bromo-2,3-dihydro-1H-indene

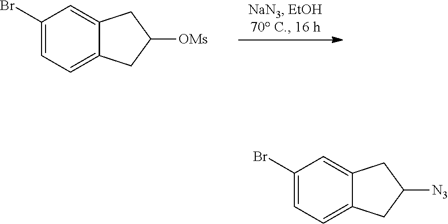 [SMILES: CS(=O)(=O)OC1Cc2ccc(Br)cc2C1 ; [N-]=[N+]=NC1Cc2ccc(Br)cc2C1]

To a stirred solution of 5-bromo-2,3-dihydro-1H-inden-2-yl methanesulfonate (8 g, 27.6 mmol, 1.0 eq) in EtOH (80 mL, 10 Vol) at room temperature, was added NaN₃ (2.7 g, 41.4 mmol, 1.5 eq) and stirred at 70° C. for 16 h. After completion of reaction by TLC, reaction mixture was quenched with water (50 mL) and extracted with EtOAc (2×50 mL). The combined organic layer was dried over Na₂SO₄, filtered and concentrated under reduced pressure to afford 2-azido-5-bromo-2,3-dihydro-1H-indene as a pale-yellow gummy liquid (6 g, Yield: Crude). TLC system: EtOAc/Hexane (30:70), R_{f} value: ˜0.5; ¹H NMR (400 MHz, CDCl₃) δ: 7.37 (s, 1H), 7.33-7.30 (m, 1H), 7.11-7.09 (m, 1H), 4.38-4.33 (m, 1H), 3.26-3.00 (m, 2H), 2.99-2.92 (m, 2H).

### Synthesis of 5-bromo-2,3-dihydro-1H-inden-2-amine

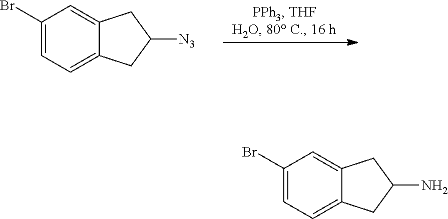 [SMILES: [N-]=[N+]=NC1Cc2ccc(Br)cc2C1 ; NC1Cc2ccc(Br)cc2C1]

To a stirred solution of 2-azido-5-bromo-2,3-dihydro-1H-indene (6 g, 25.3 mmol, 1.0 eq) in THF (120 mL, 20 Vol) at room temperature, was added PPh₃ (13 g, 50.6 mmol, 2.0 eq) followed by H₂O (1 mL, 2.0 eq) and stirred at 80° C. for 16 h. After completion of reaction by TLC, reaction mixture was acidified with 2N HCl solution (20 mL), precipitated solid was discarded by filtration and collected aq. layer was concentrated under reduced pressure to afford 5-bromo-2,3-dihydro-1H-inden-2-amine as a pale-yellow solid (6 g, Yield: Crude). TLC system: EtOAc/Hexane (100:00), R_{f} value: ˜0.1; LCMS (m/z): 212.08 (M+H)⁺.

### Synthesis of N-(5-bromo-2,3-dihydro-1H-inden-2-yl)acetamide

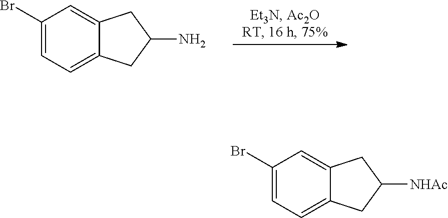 [SMILES: NC1Cc2ccc(Br)cc2C1 ; CC(=O)NC1Cc2ccc(Br)cc2C1]

To a stirred solution of 5-bromo-2,3-dihydro-1H-inden-2-amine (6 g, 28.3 mmol, 1.0 eq) in Ac₂O (30 mL, 5 Vol) at room temperature, was added Et₃N (12 mL, 84.9 mmol, 3.0 eq) and stirred for 16 h. After completion of reaction by TLC, the reaction mixture was quenched with water (30 mL) and extracted with EtOAc (2×30 mL). The combined organic layer was washed with NaHCO₃ solution (30 mL), dried over Na₂SO₄, filtered and concentrated under reduced pressure. The crude product was purified by silica gel (60-120) column chromatography [eluted 70% EtOAc in pet-ether] to afford N-(5-bromo-2,3-dihydro-1H-inden-2-yl)acetamide as off white solid (5.4 g, Yield: 75%). TLC system: EtOAc/Hexane (100:00), R_{f} value: ˜0.4; ¹H NMR (400 MHz, CDCl₃) δ: 7.37 (s, 1H), 7.31-7.28 (m, 1H), 7.09 (d, J=7.6 Hz, 1H), 5.73 (brs, 1H), 4.76-4.68 (m, 1H), 3.34-3.21 (m, 2H), 2.82-2.71 (m, 2H), 1.94 (s, 3H).

### Synthesis of N-(5-(1-hydroxycyclobutyl)-2,3-dihydro-1H-inden-2-yl)acetamide

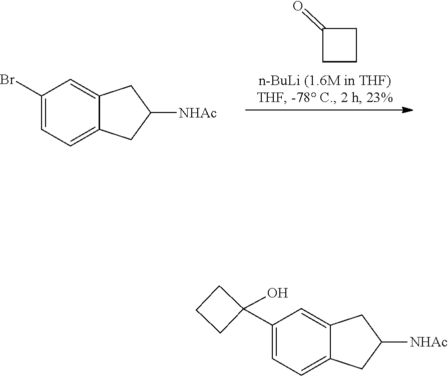 [SMILES: O=C1CCC1 ; CC(=O)NC1Cc2ccc(Br)cc2C1 ; CC(=O)NC1Cc2ccc(C3(O)CCC3)cc2C1]

To a stirred solution of N-(5-bromo-2,3-dihydro-1H-inden-2-yl)acetamide (5.4 g, 21.3 mmol, 1 eq) in THF (55 mL) at −78° C., was added n-BuLi (1.6 M in THF) (40 mL, 63.8 mmol, 3.0 eq) and stirred for 30 min. Later added cyclobutanone (2.2 g, 31.9 mmol, 1.5 eq) to reaction mixture and stirred for 2 h at −78° C. After completion of reaction by TLC, quenched with NH₄Cl solution (25 mL) and extracted with EtOAc (2×30 mL), The combined organic layer was dried over Na₂SO₄, filtered and concentrated under reduced pressure. The crude product was purified by silica gel (100-200 mesh) column chromatography [eluted with 50% EtOAc in Pet-ether] to afford N-(5-(1-hydroxycyclobutyl)-2,3-dihydro-1H-inden-2-yl)acetamide as off white solid (1.2 g, Yield: 23%), TLC system: EtOAc/Hexane (100:00), R_{f} value: ˜0.3; LCMS (m/z): 169.11 (M−OH); ¹H NMR (400 MHz, CDCl₃) δ: 7.38 (s, 1H), 7.32-7.28 (m, 2H), 5.73 (bs, 1H), 4.76-4.74 (m, 1H), 3.33-3.27 (m, 2H), 2.83-2.80 (m, 2H), 2.57-2.55 (m, 2H), 2.40-2.35 (m, 2H), 2.05-1.99 (m, 1H), 1.95 (s, 3H).

### Synthesis of 1-(2-amino-2,3-dihydro-1H-inden-5-yl)cyclobutan-1-ol

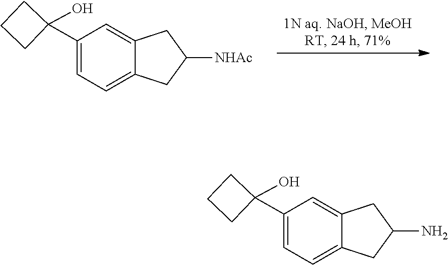 [SMILES: CC(=O)NC1Cc2ccc(C3(O)CCC3)cc2C1 ; NC1Cc2ccc(C3(O)CCC3)cc2C1]

To a stirred solution of N-(5-(1-hydroxycyclobutyl)-2,3-dihydro-1H-inden-2-yl)acetamide (1.2 g, 4.89 mmol, 1 eq) in MeOH (12 mL) was added 10% aq. NaOH (26 mL, 20 vol) and stirred at room temperature for 24 h. After completion of reaction by TLC, quenched with water (20 mL) and extracted with 10% MeOH in DCM (2×20 mL). The combined organic layer was dried over Na₂SO₄, filtered and concentrated under reduced pressure to afford crude product. The crude product was triturated with n-pentane (20 mL), diethyl ether (20 mL), decanted and dried under reduced pressure to afford 1-(2-amino-2,3-dihydro-1H-inden-5-yl)cyclobutan-1-ol as an off white solid (700 mg, yield: 71%); TLC system: MeOH:DCM (10:90), R_{f} value: ˜0.1; LCMS (m/z): 204.1 (M+H)⁺.

### Synthesis of 6-fluoro-5-((5-(1-hydroxycyclobutyl)-2,3-dihydro-1H-inden-2-yl)amino)-1-((2-(trimethylsilyl)ethoxy)methyl)-1H-pyrrolo[3,2-b]pyridine-3-carbonitrile

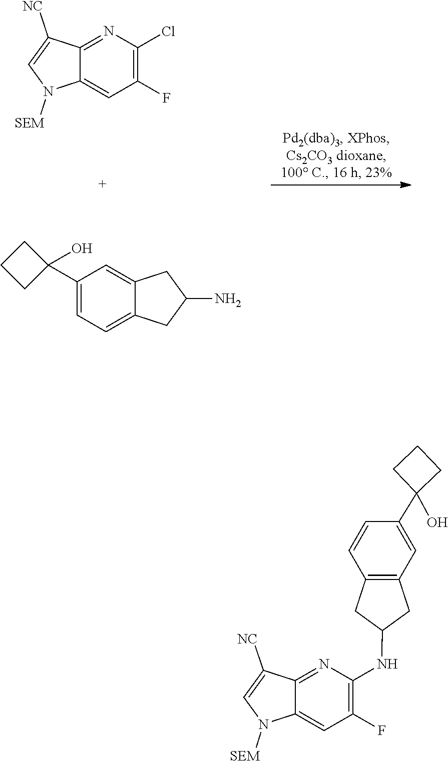 [SMILES: C[Si](C)(C)CCOCn1cc(C#N)c2nc(Cl)c(F)cc21 ; NC1Cc2ccc(C3(O)CCC3)cc2C1 ; C[Si](C)(C)CCOCn1cc(C#N)c2nc(NC3Cc4ccc(C5(O)CCC5)cc4C3)c(F)cc21]

In a sealed tube, to a degassed solution of 5-chloro-6-fluoro-1-((2-(trimethylsilyl)ethoxy)methyl)-1H-pyrrolo[3,2-b]pyridine-3-carbonitrile (1 g, 3.07 mmol, 1.0 eq) and 1-(2-amino-2,3-dihydro-1H-inden-5-yl)cyclobutan-1-ol (689 mg, 3.37 mmol, 1.1 eq) in Dioxane (10 mL) at room temperature, was added Cs₂CO₃ (2.0 g, 6.14 mmol, 2.0 eq) followed by X-Phos (293 mg, 0.61 mmol, 0.2 eq) and Pd₂(dba)₃ (281 mg, 0.31 mmol, 0.1 eq). The reaction mixture was stirred at 100° C. for 16 h. After completion of reaction by TLC, the reaction mixture was diluted with water (30 mL) and extracted with EtOAc (2×30 mL). The combined organic layer was dried over Na₂SO₄, filtered and concentrated under reduced pressure to afford crude product. The crude product was purified by silica gel [60-120] column chromatography [elution with 20% EtOAc in pet-ether] to afford 6-fluoro-5-((5-(1-hydroxycyclobutyl)-2,3-dihydro-1H-inden-2-yl)amino)-1-((2-(trimethylsilyl)ethoxy)methyl)-1H-pyrrolo[3,2-b]pyridine-3-carbonitrile as yellow gum (350 mg, Yield: 23%). TLC system: EtOAc/Hexane (30:70), R_{f} value: ˜0.3; LCMS (m/z): 493.46 (M+H)⁺; ¹H NMR (400 MHz, CDCl₃) δ: 7.59 (s, 1H), 7.40-7.32 (m, 3H), 7.26-7.25 (m, 1H), 5.36 (s, 2H), 5.07-5.06 (m, 1H), 3.53-3.49 (m, 2H), 3.47-3.42 (m, 2H), 2.96-2.91 (m, 2H), 2.60-2.55 (m, 2H), 2.41-2.36 (m, 2H), 2.03-2.00 (m, 1H), 1.70-1.67 (m, 1H), 0.90-0.86 (m, 2H), −0.04 (s, 9H).

### Synthesis of (E)-6-fluoro-N′-hydroxy-5-((5-(1-hydroxycyclobutyl)-2,3-dihydro-1H-inden-2-yl)amino)-1-((2-(trimethylsilyl)ethoxy)methyl)-1H-pyrrolo[3,2-b]pyridine-3-carboximidamide

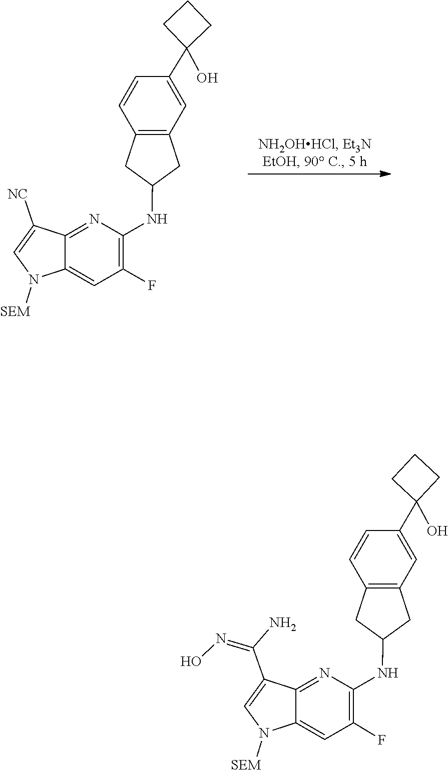 [SMILES: C[Si](C)(C)CCOCn1cc(C#N)c2nc(NC3Cc4ccc(C5(O)CCC5)cc4C3)c(F)cc21 ; C[Si](C)(C)CCOCn1cc(/C(N)=N\O)c2nc(NC3Cc4ccc(C5(O)CCC5)cc4C3)c(F)cc21]

To a stirred solution of 6-fluoro-5-((5-(1-hydroxycyclobutyl)-2,3-dihydro-1H-inden-2-yl)amino)-1-((2-(trimethylsilyl)ethoxy)methyl)-1H-pyrrolo[3,2-b]pyridine-3-carbonitrile (350 mg, 0.71 mmol, 1.0 eq) in EtOH (3.5 mL) at room temperature, was added Et₃N (215 mg, 2.13 mmol, 3.0 eq) and NH₂OH·HCl (98 mg, 1.42 mmol, 2.0 eq). The reaction mixture was stirred at 90° C. for 5 h. After completion of reaction by TLC, the reaction mixture was diluted with water (10 mL) and extracted with DCM (2×10 mL). The combined organic layer was dried over Na₂SO₄, filtered and concentrated under reduced pressure to afford (E)-6-fluoro-N′-hydroxy-5-((5-(1-hydroxycyclobutyl)-2,3-dihydro-1H-inden-2-yl)amino)-1-((2-(trimethylsilyl)ethoxy)methyl)-1H-pyrrolo[3,2-b]pyridine-3-carboximidamide as a brown solid (350 mg, Yield: Crude). TLC system: MeOH/DCM (10:90), R_{f} value: ˜0.4; LCMS (m/z): 526.49 (M+H)⁺.

### Synthesis of ethyl (E)-((6-fluoro-5-((5-(1-hydroxycyclobutyl)-2,3-dihydro-1H-inden-2-yl)amino)-1-((2-(trimethylsilyl)ethoxy)methyl)-1H-pyrrolo[3,2-b]pyridin-3-yl)(hydroxyimino)methyl)carbamate

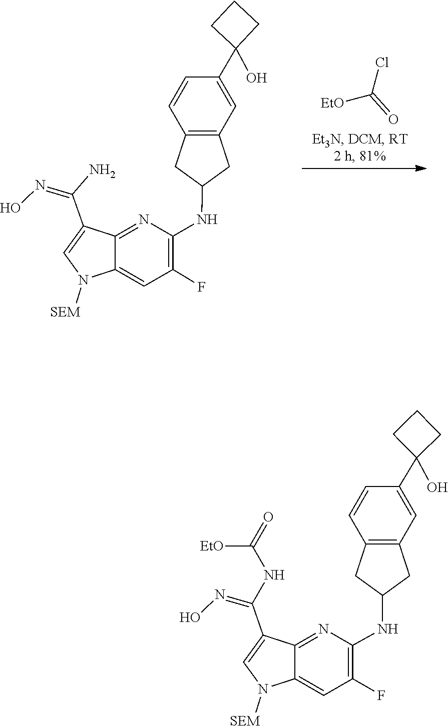 [SMILES: C[Si](C)(C)CCOCn1cc(/C(N)=N\O)c2nc(NC3Cc4ccc(C5(O)CCC5)cc4C3)c(F)cc21 ; CCOC(=O)Cl ; CCOC(=O)N/C(=N/O)c1cn(COCC[Si](C)(C)C)c2cc(F)c(NC3Cc4ccc(C5(O)CCC5)cc4C3)nc12]

To a stirred solution of (E)-6-fluoro-N′-hydroxy-5-((5-(1-hydroxycyclobutyl)-2,3-dihydro-1H-inden-2-yl)amino)-1-((2-(trimethylsilyl)ethoxy)methyl)-1H-pyrrolo[3,2-b]pyridine-3-carboximidamide (330 mg, 0.63 mmol, 1.0 eq) in DCM (3.3 mL) at 0° C., was added Et₃N (83 mg, 0.82 mmol, 1.3 eq) and Ethyl chloroformate (82 mg, 0.76 mmol, 1.2 eq). The reaction mixture was stirred at room temperature for 2 h. After completion of reaction by TLC, the reaction mixture was quenched with water (20 mL) and extracted with DCM (20 mL). The combined organic layer was dried over Na₂SO₄, filtered and concentrated to afford crude product. The crude product was purified by silica gel [60-120] column chromatography [elution with 50% EtOAc in petroleum ether] to afford ethyl (E)-((6-fluoro-5-((5-(1-hydroxycyclobutyl)-2,3-dihydro-1H-inden-2-yl)amino)-1-((2-(trimethylsilyl)ethoxy)methyl)-1H-pyrrolo[3,2-b]pyridin-3-yl)(hydroxyimino)methyl)carbamate as a yellow solid (300 mg, Yield: 81%). TLC system: EtOAc/Hexane (50:50), R_{f} value: ˜0.5; LCMS (m/z): 598.45 (M+H)⁺. ¹H NMR (400 MHz, CDCl₃) δ: 7.98 (brs, 1H), 7.45 (d, J=10.8 Hz, 1H), 7.40 (s, 1H), 7.36-7.31 (m, 1H), 5.37 (s, 2H), 4.98-4.97 (m, 1H), 4.34 (q, J=7.2 Hz, 2H), 3.47-3.41 (m, 4H), 3.05-2.98 (m, 2H), 2.60-2.55 (m, 2H), 2.39-2.36 (m, 2H), 2.07-2.04 (m, 1H), 1.75-1.66 (m, 2H), 1.36 (t, J=7.2 Hz, 3H), 0.89-0.85 (m, 2H), −0.05 (s, 9H).

### Synthesis of 3-(6-fluoro-5-((5-(1-hydroxycyclobutyl)-2,3-dihydro-1H-inden-2-yl)amino)-1-((2-(trimethylsilyl)ethoxy)methyl)-1H-pyrrolo[3,2-b]pyridin-3-yl)-1,2,4-oxadiazol-5(4H)-one

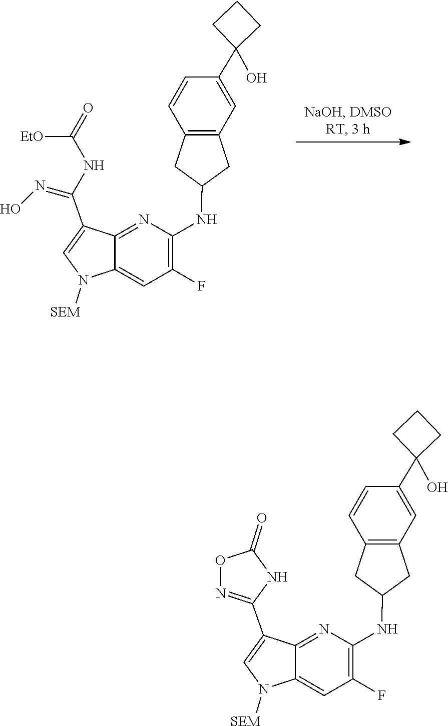 [SMILES: CCOC(=O)N/C(=N/O)c1cn(COCC[Si](C)(C)C)c2cc(F)c(NC3Cc4ccc(C5(O)CCC5)cc4C3)nc12 ; C[Si](C)(C)CCOCn1cc(-c2noc(=O)[nH]2)c2nc(NC3Cc4ccc(C5(O)CCC5)cc4C3)c(F)cc21]

To a stirred solution of ethyl (E)-((6-fluoro-5-((5-(1-hydroxycyclobutyl)-2,3-dihydro-1H-inden-2-yl)amino)-1-((2-(trimethylsilyl)ethoxy)methyl)-1H-pyrrolo[3,2-b]pyridin-3-yl)(hydroxyimino)methyl)carbamate (300 mg, 0.5 mmol, 1.0 eq) in DMSO (3.5 Vol, 1.0 mL) at room temperature, was added NaOH (24 mg, 0.6 mmol, 1.2 eq) and reaction mixture was stirred at room temperature for 3 h. After completion of reaction by TLC, the reaction mixture was quenched with 2N HCl solution (5 mL), precipitated solid was filtered, washed with n-pentane (5 mL) and dried under vacuum to afford 3-(6-fluoro-5-((5-(1-hydroxycyclobutyl)-2,3-dihydro-1H-inden-2-yl)amino)-1-((2-(trimethylsilyl)ethoxy)methyl)-1H-pyrrolo[3,2-b]pyridin-3-yl)-1,2,4-oxadiazol-5(4H)-one as a pale brown solid (250 mg, Yield: Crude). TLC system: EtOAc/Hexane (100:00), R_{f} value: ˜0.5; LCMS (m/z): 550.42 (M−H)⁻.

### Synthesis of 3-(6-fluoro-5-((5-(1-hydroxycyclobutyl)-2,3-dihydro-1H-inden-2-yl)amino)-1H-pyrrolo[3,2-b]pyridin-3-yl)-1,2,4-oxadiazol-5(4H)-one

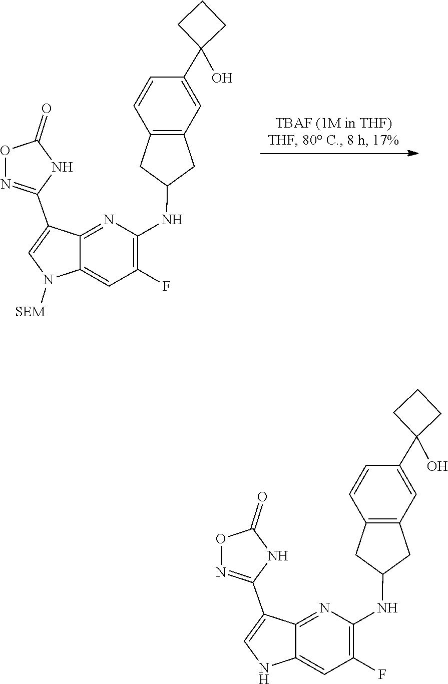 [SMILES: C[Si](C)(C)CCOCn1cc(-c2noc(=O)[nH]2)c2nc(NC3Cc4ccc(C5(O)CCC5)cc4C3)c(F)cc21 ; O=c1[nH]c(-c2c[nH]c3cc(F)c(NC4Cc5ccc(C6(O)CCC6)cc5C4)nc23)no1]

To a stirred solution of 3-(6-fluoro-5-((5-(1-hydroxycyclobutyl)-2,3-dihydro-1H-inden-2-yl)amino)-1-((2-(trimethylsilyl)ethoxy)methyl)-1H-pyrrolo[3,2-b]pyridin-3-yl)-1,2,4-oxadiazol-5(4H)-one (250 mg, 0.45 mmol, 1.0 eq) in THF (2.5 mL) at 0° C., was added TBAF (1.0 M in THF) (4.53 mL, 4.53 mmol, 10 eq) and stirred at 80° C. for 8 h. After completion of reaction by TLC, the reaction mixture was quenched with NH₄Cl solution (20 mL) and extracted with 10% MeOH in DCM (2×20 mL). The combined organic layer was dried over Na₂SO₄, filtered and concentrated under reduced pressure. Obtained crude was purified by reverse phase column chromatography [elution with 46% ACN in 0.1% FA in H₂O] followed by Prep-HPLC and collected fractions were lyophilized to afford 3-(6-fluoro-5-((5-(1-hydroxycyclobutyl)-2,3-dihydro-1H-inden-2-yl)amino)-1H-pyrrolo[3,2-b]pyridin-3-yl)-1,2,4-oxadiazol-5(4H)-one as off white solid (33 mg, Yield: 17%). TLC system: MeOH/DCM (10:90), R_{f} value: ˜0.4; LCMS (m/z): 420.19 (M−H)⁻; 1H NMR (400 MHz, DMSO-d₆) δ: 12.05 (s, 1H), 11.71 (s, 1H), 7.87 (d, J=3.2 Hz, 1H), 7.61 (d, J=11.6 Hz, 1H), 7.33 (s, 1H), 7.27-7.25 (m, 1H), 7.17 (d, J=8.0 Hz, 1H), 6.60 (d, J=6.0 Hz, 1H), 5.36 (s, 1H), 5.00-4.94 (m, 1H), 3.37-3.33 (m, 2H), 2.95-2.88 (m, 2H), 2.39-2.34 (m, 2H), 2.28-2.20 (m, 2H), 1.90-1.87 (m, 1H), 1.64-1.59 (m, 1H).

Prep-HPLC condition: Column X-Bridge C18, (250*19.0 mm) 5u Buffer A: 0.1% FA IN WATER; B: A CN:H2O (80:20) Gradient (T/% B) 0/50, 15/65, 15.1/98 Flow Rate 12 ml/min Solubility ACN+THF.

### SYNTHETIC EXAMPLE 40

### Synthesis of Compound I-66

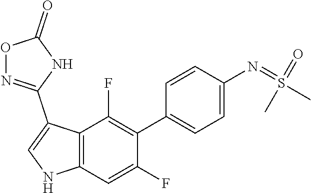 [SMILES: CS(C)(=O)=Nc1ccc(-c2c(F)cc3[nH]cc(-c4noc(=O)[nH]4)c3c2F)cc1]

### Synthesis of ((4-bromophenyl) imino) dimethyl-λ⁶-sulfanone

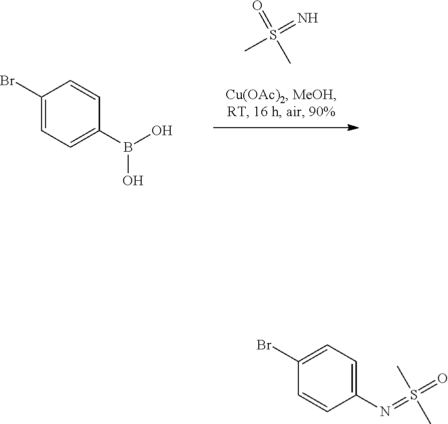

To a stirred solution iminodimethyl-λ⁶-sulfanone (5 g, 53.7 mmol, 1 eq) and (4-bromophenyl) boronic acid (220 mL, 107.4 mmol, 1.6 eq) in MeOH (50 mL) at room temperature was added Cu(OAc)₂ (0.98 g, 5.36 mmol, 0.1 eq) and stirred for 16 h under air atmosphere. After completion of reaction by TLC, reaction mixture was concentrated and purified by silica gel (100-200 mesh) column chromatography [elution with 30% EtOAc/Hexane] to afford ((4-bromophenyl) imino) dimethyl-λ⁶-sulfanone as off white solid (12 g, Yield: 90%), TLC system: EtOAc: hexane (50:50), R_{f} value: ˜0.3; LCMS (m/z): 248.04 (M+H).±¹H NMR (400 MHz, CDCl₃). 7.34 (dd, J=2.4 & 6.8 Hz, 2H), 6.98 (dd, J=2.4 & 6.8 Hz, 2H), 3.18 (s, 6H).

### Synthesis of dimethyl ((4-(4,4,5,5-tetramethyl-1,3,2-dioxaborolan-2-yl)phenyl) imino)-λ⁶-sulfanone

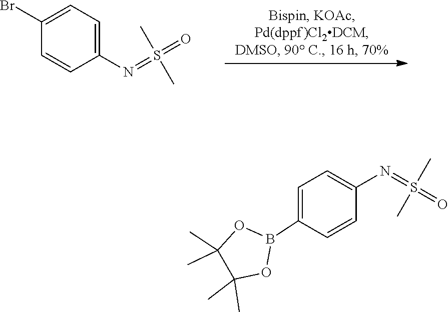 [SMILES: CS(C)(=O)=Nc1ccc(Br)cc1 ; CC1(C)OB(c2ccc(N=S(C)(C)=O)cc2)OC1(C)C]

In a sealed tube, to a degassed solution of ((4-bromophenyl) imino) dimethyl-16-sulfanone (6 g, 24.3 mmol, 1.0 eq) and B₂(pin)₂ (9.3 g, 36.4 mmol, 1.5 eq) in DMSO (120 mL) at room temperature, was added KOAc (7 g, 72.9 mmol, 3.0 eq) followed by Pd(dppf)Cl₂·DCM (2 g, 2.43 mmol, 0.1 eq) and stirred at 90° C. for 16 h. After completion of reaction by TLC, reaction mixture was filtered through a pad of diatomaceous earth (e.g., Celite®), washed with EtOAc (100 mL), collected filtrate was diluted with water (100 mL) and extracted with EtOAc (2×20 mL). The combined organic layer was dried over Na₂SO₄, filtered and concentrated under reduced pressure. Obtained crude was purified by silica gel (100-200 mesh) column chromatography [elution with 20% EtOAc/Hexane] to afford dimethyl ((4-(4,4,5,5-tetramethyl-1,3,2-dioxaborolan-2-yl)phenyl) imino)-λ⁶-sulfanone as light brown solid (5 g, Yield: 70%). TLC system: TLC system: EtOAc: hexane (50:50), R_{f} value: ˜0.25; ¹H NMR (400 MHz, CDCl₃) δ: 7.69 (d, J=8.4 Hz, 2H), 7.07 (d, J=8.4 Hz, 2H), 3.17 (s, 6H), 1.33 (s, 12H).

### Synthesis of 3-(5-(4-((dimethyl(oxo)-λ⁶-sulfaneylidene)amino)phenyl)-4,6-difluoro-1H-indol-3-yl)-1,2,4-oxadiazol-5(4H)-one

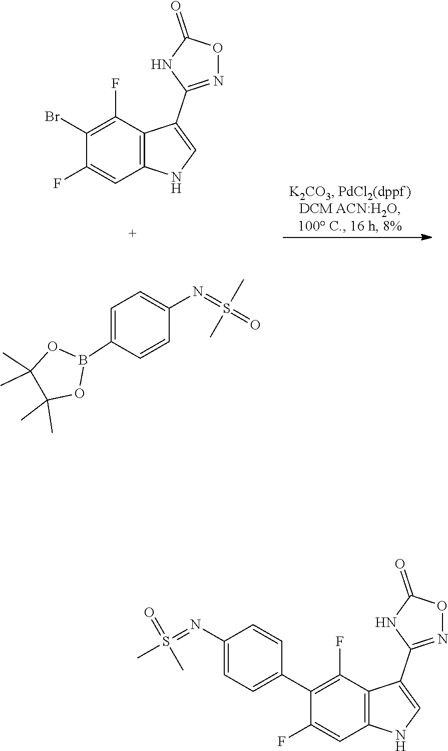 [SMILES: O=c1[nH]c(-c2c[nH]c3cc(F)c(Br)c(F)c23)no1 ; CC1(C)OB(c2ccc(N=S(C)(C)=O)cc2)OC1(C)C ; CS(C)(=O)=Nc1ccc(-c2c(F)cc3[nH]cc(-c4noc(=O)[nH]4)c3c2F)cc1]

In a sealed tube, to a degassed solution of 3-(5-bromo-4,6-difluoro-1H-indol-3-yl)-1,2,4-oxadiazol-5(4H)-one (300 mg, 0.95 mmol, 1.0 eq) and dimethyl ((4-(4,4,5,5-tetramethyl-1,3,2-dioxaborolan-2-yl)phenyl) imino)-λ⁶-sulfanone (562 mg, 1.90 mmol, 2 eq) in ACN:H₂O (8:2, 8 mL) at room temperature, was added K₂CO₃ (395 mg, 2.85 mmol, 3 eq) followed by Pd(dppf)Cl₂·DCM (78 mg, 0.09 mmol, 0.1 eq) and stirred at 100° C. for 16 h. After completion of reaction by TLC, reaction mixture was diluted with water (20 mL) and washed with EtOAc (2×20 mL). Aqueous layer was evaporated 90% of volume, resulting solid was filtered & dried to provide semi pure compound. This was purified by Prep-HPLC and collected fractions were lyophilized to afford 3-(5-(4-((dimethyl(oxo)-λ⁶-sulfaneylidene)amino)phenyl)-4,6-difluoro-1H-indol-3-yl)-1,2,4-oxadiazol-5(4H)-one as off white solid (30 mg, Yield: 8%). TLC system: EtOAc (100%), R_{f} value: ˜0.2; LCMS (m/z): 403.17 (M+H)⁺. ¹H NMR (400 MHz, DMSO-d₆) δ: 12.50 (br s, 1H), 12.26 (s, 1H), 7.99 (d, J=2.4 Hz, 1H), 7.32-7.26 (m, 3H), 7.05-7.02 (m, 2H), 3.27 (s, 6H).

Prep Conditions: Sample code: C0698-82-CR; Column Gemini C18 (250*21.2) mm, 5mic; Buffer MP-(A): 0.1% FA IN WATER, MP-(B): Acetonitrile; Gradient (T/% B) 0/25, 15/50, 25/98; Flow Rate: 15 ml/min: Solubility ACN, THF.

### SYNTHETIC EXAMPLE 41

### Synthesis of Compound I-67

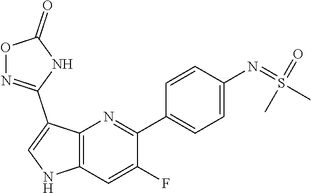 [SMILES: CS(C)(=O)=Nc1ccc(-c2nc3c(-c4noc(=O)[nH]4)c[nH]c3cc2F)cc1]

### Synthesis of 5-(4-((dimethyl(oxo)-16-sulfaneylidene)amino)phenyl)-6-fluoro-1-((2-(trimethylsilyl)ethoxy)methyl)-1H-pyrrolo[3,2-b]pyridine-3-carbonitrile

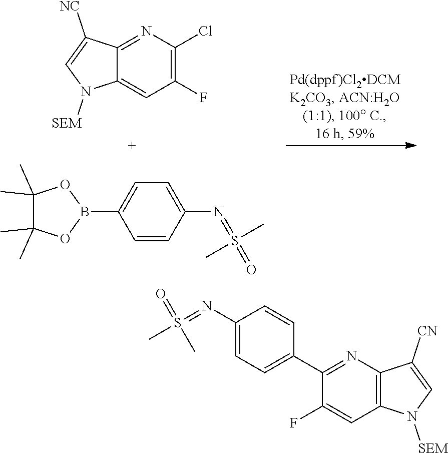

In a sealed tube to a degassed solution of 5-chloro-6-fluoro-1-((2-(trimethylsilyl)ethoxy)methyl)-1H-pyrrolo[3,2-b]pyridine-3-carbonitrile (600 mg, 1.85 mmol, 1.0 eq) and dimethyl ((4-(4,4,5,5-tetramethyl-1,3,2-dioxaborolan-2-yl)phenyl) imino)-16-sulfanone (820 mg, 2.77 mmol, 1.5 eq) in ACN:H₂O (1:1, 6 mL) at room temperature, was added K₂CO₃ (766 mg, 5.55 mmol, 3.0 eq) followed by Pd(dppf)Cl₂·DCM (151 mg, 0.18 mmol, 0.1 eq) and stirred at 100° C. for 16 h. After completion of reaction by TLC, the reaction mixture was diluted with water (20 mL) and extracted with EtOAc (2×20 mL). The combined organic layer was dried over Na₂SO₄, filtered and concentrated under reduced pressure. Crude product was purified by silica gel [100-200] column chromatography [elution with 70% EtOAc in petroleum ether] to afford 5-(4-((dimethyl(oxo)-16-sulfaneylidene)amino)phenyl)-6-fluoro-1-((2-(trimethylsilyl)ethoxy)methyl)-1H-pyrrolo[3,2-b]pyridine-3-carbonitrile as pale brown gum (500 mg, Yield: 59%). TLC system: EtOAc/Hexane (100:00), R_{f} value: ˜0.6; LCMS (m/z): 459.40 (M+H)⁺; ¹H NMR (400 MHz, CDCl₃) δ: 8.01 (dd, J=2.0 Hz, 8.8 Hz, 2H), 7.87 (s, 1H), 7.66 (d, J=10.8 Hz, 1H), 7.25-7.23 (m, 2H), 5.50 (s, 2H), 3.55-3.51 (m, 2H), 3.24 (s, 6H), 0.96-0.92 (m, 2H), −0.02 (s, 9H).

### Synthesis of (Z)-5-(4-((dimethyl(oxo)-16-sulfaneylidene)amino)phenyl)-6-fluoro-N′-hydroxy-1-((2-(trimethylsilyl)ethoxy)methyl)-1H-pyrrolo[3,2-b]pyridine-3-carboximidamide

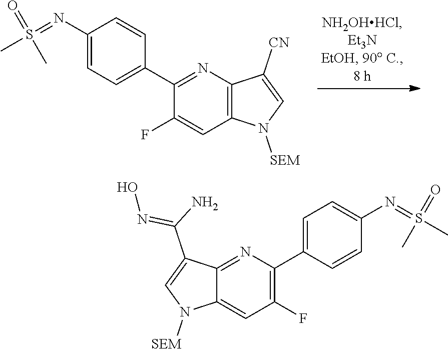 [SMILES: C[Si](C)(C)CCOCn1cc(C#N)c2nc(-c3ccc(N=S(C)(C)=O)cc3)c(F)cc21 ; C[Si](C)(C)CCOCn1cc(/C(N)=N/O)c2nc(-c3ccc(N=S(C)(C)=O)cc3)c(F)cc21]

To a stirred solution of 5-(4-((dimethyl(oxo)-16-sulfaneylidene)amino)phenyl)-6-fluoro-1-((2-(trimethylsilyl)ethoxy)methyl)-1H-pyrrolo[3,2-b]pyridine-3-carbonitrile (400 mg, 0.87 mmol, 1.0 eq) in EtOH (4 mL) at room temperature, was added Et₃N (264 mg, 2.61 mmol, 3.0 eq) and NH₂OH·HCl (120 mg, 1.74 mmol, 2.0 eq). The reaction mixture was stirred at 90° C. for 8 h. After completion of reaction by TLC, the reaction mixture was diluted with water (15 mL) and extracted with DCM (2×15 mL). The combined organic layer was dried over Na₂SO₄, filtered and concentrated under reduced pressure to afford (Z)-5-(4-((dimethyl(oxo)-16-sulfaneylidene)amino)phenyl)-6-fluoro-N′-hydroxy-1-((2-(trimethylsilyl)ethoxy)methyl)-1H-pyrrolo[3,2-b]pyridine-3-carboximidamide as a brown gum (400 mg, Yield: Crude). TLC system: MeOH/DCM (10:90), R_{f} value: ˜0.4; LCMS (m/z): 492.50 (M+H)⁺.

### Synthesis of ethyl (Z)-((5-(4-((dimethyl(oxo)-16-sulfaneylidene)amino)phenyl)-6-fluoro-1-((2-(trimethylsilyl)ethoxy)methyl)-1H-pyrrolo[3,2-b]pyridin-3-yl)(hydroxyimino)methyl)carbamate

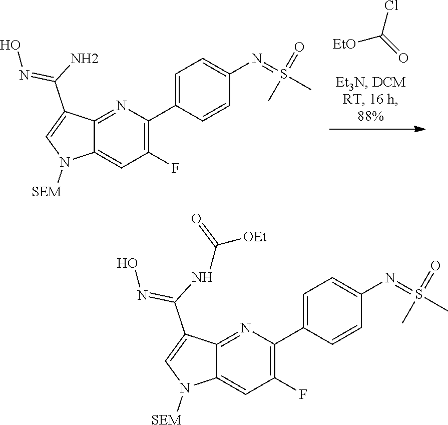 [SMILES: C[Si](C)(C)CCOCn1cc(/C(N)=N/O)c2nc(-c3ccc(N=S(C)(C)=O)cc3)c(F)cc21 ; CCOC(=O)Cl ; CCOC(=O)N/C(=N\O)c1cn(COCC[Si](C)(C)C)c2cc(F)c(-c3ccc(N=S(C)(C)=O)cc3)nc12]

To a stirred solution of (Z)-5-(4-((dimethyl(oxo)-16-sulfaneylidene)amino)phenyl)-6-fluoro-N′-hydroxy-1-((2-(trimethylsilyl)ethoxy)methyl)-1H-pyrrolo[3,2-b]pyridine-3-carboximidamide (400 mg, 0.81 mmol, 1.0 eq) in DCM (4 mL) at 0° C., was added Et₃N (106 mg, 1.05 mmol, 1.3 eq) and Ethyl chloroformate (106 mg, 0.98 mmol, 1.2 eq). The reaction mixture was stirred at room temperature for 16 h. After completion of reaction by TLC, the reaction mixture was quenched with water (15 mL) and extracted with DCM (2×mL). The combined organic layer was dried over Na₂SO₄, filtered and concentrated to afford crude product. The crude product was purified by silica gel [60-120] column chromatography [elution with 50% EtOAc in petroleum ether] to afford ethyl (Z)-((5-(4-((dimethyl(oxo)-16-sulfaneylidene)amino)phenyl)-6-fluoro-1-((2-(trimethylsilyl)ethoxy)methyl)-1H-pyrrolo[3,2-b]pyridin-3-yl) (hydroxyimino)methyl)carbamate as yellow solid (400 mg, Yield: 88%). TLC system: EtOAc/Hexane (100:0), R_{f} value: ˜0.4; ¹H NMR (400 MHz, CDCl₃) δ: 8.19 (s, 1H), 7.89 (dd, J=2.0 Hz, 8.8 Hz, 2H), 7.69 (d, J=11.2 Hz, 1H), 7.24 (d, J=8.8 Hz, 2H), 5.49 (s, 2H), 4.37 (q, J=7.2 Hz, 2H), 3.52 (t, J=8.0 Hz, 2H), 3.26 (s, 6H), 1.43 (t, J=7.2 Hz, 3H), 0.96-0.93 (m, 2H), −0.02 (s, 9H).

### Synthesis of 3-(5-(4-((dimethyl(oxo)-16-sulfaneylidene)amino)phenyl)-6-fluoro-1-((2-(trimethylsilyl)ethoxy)methyl)-1H-pyrrolo[3,2-b]pyridin-3-yl)-1,2,4-oxadiazol-5(4H)-one

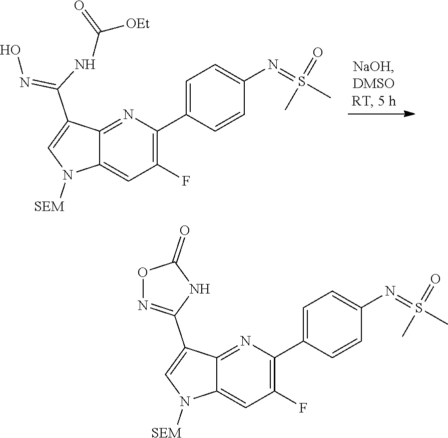 [SMILES: CCOC(=O)N/C(=N\O)c1cn(COCC[Si](C)(C)C)c2cc(F)c(-c3ccc(N=S(C)(C)=O)cc3)nc12 ; C[Si](C)(C)CCOCn1cc(-c2noc(=O)[nH]2)c2nc(-c3ccc(N=S(C)(C)=O)cc3)c(F)cc21]

To a stirred solution of ethyl (Z)-((5-(4-((dimethyl(oxo)-16-sulfaneylidene)amino)phenyl)-6-fluoro-1-((2-(trimethylsilyl)ethoxy)methyl)-1H-pyrrolo[3,2-b]pyridin-3-yl)(hydroxyimino)methyl)carbamate (400 mg, 0.71 mmol, 1.0 eq) in DMSO (3.5 Vol, 1.4 mL) at room temperature, was added NaOH (34 mg, 0.85 mmol, 1.2 eq) and reaction mixture was stirred at room temperature for 5 h. After completion of reaction by TLC, the reaction mixture was quenched with 2N HCl solution (5 mL), diluted with water (5 mL) and extracted with 10% MeOH in DCM (2×20 mL). The combined organic layer was dried over Na₂SO₄, filtered and concentrated to afford 3-(5-(4-((dimethyl(oxo)-16-sulfaneylidene)amino)phenyl)-6-fluoro-1-((2-(trimethylsilyl)ethoxy)methyl)-1H-pyrrolo[3,2-b]pyridin-3-yl)-1,2,4-oxadiazol-5(4H)-one as brown liquid (350 mg, Yield: Crude). TLC system: MeOH/DCM (10:90), R_{f} value: ˜0.3; LCMS (m/z): 518.45 (M+H)⁺.

### Synthesis of 3-(5-(4-((dimethyl(oxo)-16-sulfaneylidene)amino)phenyl)-6-fluoro-1H-pyrrolo[3,2-b]pyridin-3-yl)-1,2,4-oxadiazol-5(4H)-one

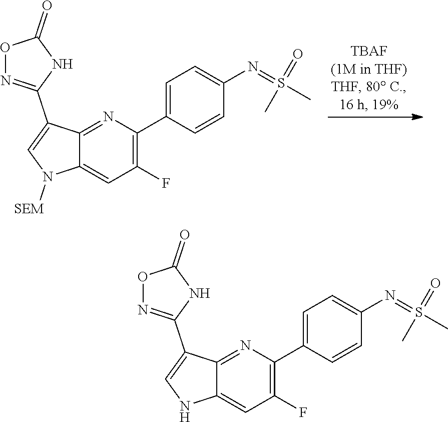 [SMILES: C[Si](C)(C)CCOCn1cc(-c2noc(=O)[nH]2)c2nc(-c3ccc(N=S(C)(C)=O)cc3)c(F)cc21 ; CS(C)(=O)=Nc1ccc(-c2nc3c(-c4noc(=O)[nH]4)c[nH]c3cc2F)cc1]

To a stirred solution of 3-(5-(4-((dimethyl(oxo)-16-sulfaneylidene)amino)phenyl)-6-fluoro-1-((2-(trimethylsilyl)ethoxy)methyl)-1H-pyrrolo[3,2-b]pyridin-3-yl)-1,2,4-oxadiazol-5(4H)-one (350 mg, 0.68 mmol, 1.0 eq) in THF (3.5 mL) at 0° C., was added TBAF (1.0 M in THF) (6.7 mL, 6.76 mmol, 10 eq) and stirred at 80° C. for 16 h. After completion of reaction by TLC, the reaction mixture was diluted with water (20 mL) and extracted with 10% MeOH in DCM (2×20 mL). The combined organic layer was dried over Na₂SO₄, filtered and concentrated under reduced pressure. The crude product was purified by reverse phase column chromatography [elution 60% ACN in 0.1% FA in H₂O] to afford desired product with TBAF residues, which was re-purified by prep-HPLC and fractions were feezed, lyophilized to afford 3-(5-(4-((dimethyl(oxo)-16-sulfaneylidene)amino)phenyl)-6-fluoro-1H-pyrrolo[3,2-b]pyridin-3-yl)-1,2,4-oxadiazol-5(4H)-one as white solid (35 mg, Yield: 19%). TLC system: MeOH/DCM (10:90), R_{f} value: ˜0.1; LCMS (m/z): 388.32 (M+H)⁺; ¹H NMR (400 MHz, DMSO-d₆) δ: 12.10 (s, 1H), 8.18 (s, 1H), 7.91-7.83 (m, 3H), 7.07-7.05 (m, 2H), 3.28 (s, 6H).

Prep-HPLC condition: Column GEMINI (250*21.2) mm 5mic Buffer MP-(A): 10 mM ABC in water MP-(B): Acetonitrile; water (80:20) Gradient (T/% B) 0/20, 15/55, 18/95, 25/98 Flow Rate 13 ml/min.

### SYNTHETIC EXAMPLE 42

### Synthesis of Compound I-68

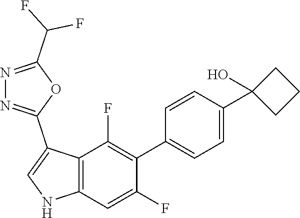 [SMILES: OC1(c2ccc(-c3c(F)cc4[nH]cc(-c5nnc(C(F)F)o5)c4c3F)cc2)CCC1]

### Synthesis of 5-bromo-4,6-difluoro-1H-indole-3-carbaldehyde

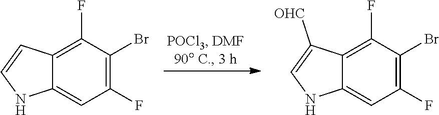 [SMILES: Fc1cc2[nH]ccc2c(F)c1Br ; O=Cc1c[nH]c2cc(F)c(Br)c(F)c12]

To a stirred solution of POCl₃ (6.7 mL, 72.2 mmol, 2.0 eq) in DMF (6.1 mL, 75.6 mmol, 2.1 eq) at room temperature, was added a solution of 5-bromo-4,6-difluoro-1H-indole (7.0 g, 36.1 mmol, 1.0 eq) in DMF (7 mL). The reaction mixture was stirred at 90° C. for 3 h. After completion of reaction by TLC, the reaction mixture was treated with 1N aq. NaOH solution (50 mL), obtained solid was filtered, washed with n-pentane (50 mL) and dried under vacuum to afford 5-bromo-4,6-difluoro-1H-indole-3-carbaldehyde as brick red solid (6 g, Yield: 64%). TLC system: EtOAc/Hexane (50:50), R_{f} value: ˜0.2; LCMS (m/z): 260.05 (M+H)⁺.

### Synthesis of 5-bromo-4,6-difluoro-1-((2-(trimethylsilyl)ethoxy)methyl)-1H-indole-3-carbaldehyde

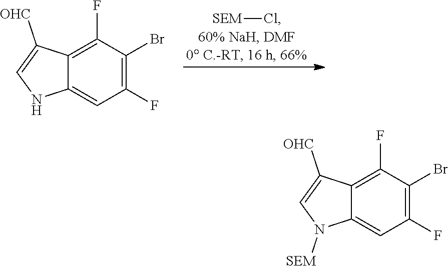 [SMILES: O=Cc1c[nH]c2cc(F)c(Br)c(F)c12 ; C[Si](C)(C)CCOCCl ; C[Si](C)(C)CCOCn1cc(C=O)c2c(F)c(Br)c(F)cc21]

To a stirred solution of 5-bromo-4,6-difluoro-1H-indole-3-carbaldehyde (6 g, 23.2 mmol, 1.0 eq) in DMF (120 mL) at 0° C., was added NaH (1.11 g, 46.3 mmol, 2.0 eq) and stirred for 30 min. Then SEM-Cl (6.4 mL, 34.7 mmol, 1.5 eq) was added to reaction mixture and stirred at room temperature for 16 h. After completion of reaction by TLC, the reaction mixture was quenched with ice cold water (50 mL) and extracted with EtOAc (2×50 mL). The combined organic layer was dried over Na₂SO₄, filtered and concentrated under reduced pressure to afford crude product. The crude product was purified by silica gel [100-200] column chromatography [elution with 30% EtOAc in petroleum ether] to afford 5-bromo-4,6-difluoro-1-((2-(trimethylsilyl)ethoxy)methyl)-1H-indole-3-carbaldehyde as a colorless solid (5.3 g, Yield: 66%). TLC system: EtOAc/Hexane (30:70), R_{f} value: ˜0.4; LCMS (m/z): 390.18 (M+H)⁺; ¹H NMR (400 MHz, CDCl₃) δ: 10.25 (s, 1H), 7.93 (s, 1H), 7.24 (d, J=8.0 Hz, 1H), 5.51 (s, 2H), 3.55-3.50 (m, 2H), 0.96-0.92 (m, 2H), −0.03 (s, 9H).

### Synthesis of 5-bromo-4,6-difluoro-1-((2-(trimethylsilyl)ethoxy)methyl)-1H-indole-3-carboxylic acid

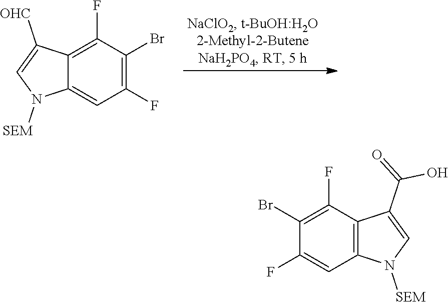 [SMILES: C[Si](C)(C)CCOCn1cc(C=O)c2c(F)c(Br)c(F)cc21 ; C[Si](C)(C)CCOCn1cc(C(=O)O)c2c(F)c(Br)c(F)cc21]

To a stirred solution of 5-bromo-4,6-difluoro-1-((2-(trimethylsilyl)ethoxy)methyl)-1H-indole-3-carbaldehyde (5.3 g, 13.6 mmol, 1.0 eq) in t-BuOH (26.5 mL) and H₂O (26.5 mL) at room temperature, was added NaClO₂ (12.3 g, 136.2 mmol, 10 eq), NaH₂PO₄ (16.3 g, 136.2 mmol, 10 eq) followed by 2-methyl-2-butene (29 mL, 272.4 mmol, 20 eq) and stirred for 5 h at room temperature. After completion of reaction by TLC, the reaction mixture was diluted with water (30 mL) and extracted with DCM (2×30 mL). The combined organic layer was dried over Na₂SO₄, filtered and concentrated under reduced pressure to afford 5-bromo-4,6-difluoro-1-((2-(trimethylsilyl)ethoxy)methyl)-1H-indole-3-carboxylic acid as a colorless solid (5.4 g, Yield: Crude). TLC system: EtOAc/Hexane (50:50), R_{f} value: ˜0.2; LCMS (m/z): 406.26 (M+H)⁺; ¹H NMR (400 MHz, CDCl₃) δ: 7.97 (s, 1H), 7.18 (dd, J=1.2 Hz, 8.0 Hz, 1H), 5.46 (s, 2H), 3.49 (t, J=8.4 Hz, 2H), 0.91 (t, J=8.0 Hz, 2H), −0.04 (s, 9H).

### Synthesis of methyl 5-bromo-4,6-difluoro-1-((2-(trimethylsilyl)ethoxy)methyl)-1H-indole-3-carboxylate

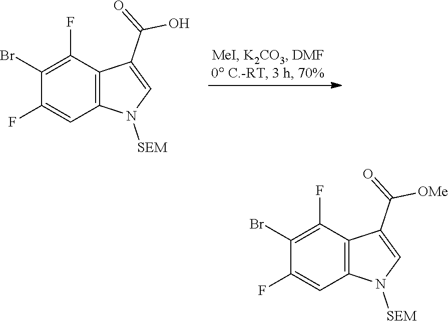 [SMILES: C[Si](C)(C)CCOCn1cc(C(=O)O)c2c(F)c(Br)c(F)cc21 ; COC(=O)c1cn(COCC[Si](C)(C)C)c2cc(F)c(Br)c(F)c12]

To a stirred solution of 5-bromo-4,6-difluoro-1-((2-(trimethylsilyl)ethoxy)methyl)-1H-indole-3-carboxylic acid (5.4 g, 13.3 mmol, 1.0 eq) in DMF (20 Vol) at 0° C., was added K₂CO₃ (3.68 g, 26.6 mmol, 2.0 eq), MeI (3.78 g, 26.6 mmol, 2.0 eq) and stirred for 3 h at room temperature. After completion of reaction by TLC, the reaction mixture was diluted with water (50 mL) and extracted with EtOAc (2×50 mL). The combined organic layer was washed with brine solution (50 mL), dried over Na₂SO₄, filtered and concentrated under reduced pressure to afford methyl 5-bromo-4,6-difluoro-1-((2-(trimethylsilyl)ethoxy)methyl)-1H-indole-3-carboxylate as a white solid (4.0 g, Yield: 70% in 2 steps). TLC system: EtOAc/Hexane (20:80), R_{f} value: ˜0.5; LCMS (m/z): 420.23 (M+H)⁺; ¹H NMR (400 MHz, CDCl₃) δ: 7.90 (s, 1H), 7.20 (dd, J=1.2 Hz, 8.0 Hz, 1H), 5.48 (s, 2H), 3.94 (s, 3H), 3.53-3.49 (m, 2H), 0.96-0.92 (m, 2H), −0.04 (s, 9H).

### Synthesis of methyl 4,6-difluoro-5-(4-(1-hydroxycyclobutyl)phenyl)-1-((2-(trimethylsilyl)ethoxy)methyl)-1H-indole-3-carboxylate

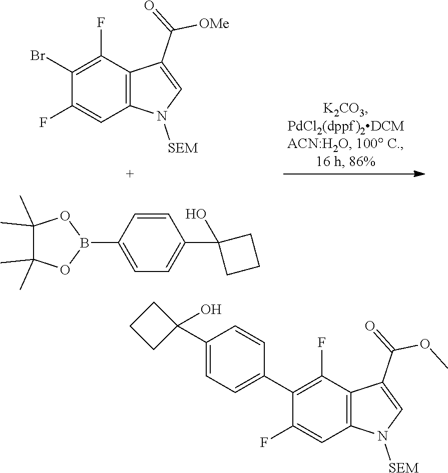

In a sealed tube, to a degassed solution of methyl 5-bromo-4,6-difluoro-1-((2-(trimethylsilyl)ethoxy)methyl)-1H-indole-3-carboxylate (500 mg, 1.19 mmol, 1.0 eq) and 1-(4-(4,4,5,5-tetramethyl-1,3,2-dioxaborolan-2-yl)phenyl)cyclobutan-1-ol (490 mg, 1.78 mmol, 1.5 eq) in ACN:H₂O (1:1, 10 Vol) at room temperature, was added K₂CO₃ (493 mg, 3.57 mmol, 3.0 eq) followed by Pd(dppf)Cl₂·DCM (97 mg, 0.12 mmol, 0.1 eq) and stirred at 100° C. for 16 h. One more batch of same scale was repeated and after completion of reaction by TLC, both the reactions combined, diluted with water (30 mL) and extracted with EtOAc (2×50 mL). The combined organic layer was dried over Na₂SO₄, filtered and concentrated under reduced pressure to afford crude product. The crude product was purified by silica gel [100-200 mesh] column chromatography [elution with 20% EtOAc in petroleum ether] to afford methyl 4,6-difluoro-5-(4-(1-hydroxycyclobutyl)phenyl)-1-((2-(trimethylsilyl)ethoxy)methyl)-1H-indole-3-carboxylate as a pale yellow gum (1 g, Yield: 86%). TLC system: EtOAc/Hexane (30:70), R_{f} value: ˜0.5; LCMS (m/z): 488.31 (M+H)⁺; ¹H NMR (400 MHz, CDCl₃) δ: 7.91 (s, 1H), 7.63 (d, J=8.4 Hz, 2H), 7.55 (d, J=8.0 Hz, 2H), 7.18 (d, J=8.8 Hz, 1H), 5.48 (s, 2H), 3.92 (s, 3H), 3.53 (t, J=8.4 Hz, 2H), 2.69-2.62 (m, 2H), 2.48-2.40 (m, 2H), 2.09-2.06 (m, 1H), 1.81-1.76 (m, 1H), 0.97-0.93 (m, 2H), −0.03 (s, 9H).

### Synthesis of 4,6-difluoro-5-(4-(1-hydroxycyclobutyl)phenyl)-1-((2-(trimethylsilyl)ethoxy)methyl)-1H-indole-3-carbohydrazide

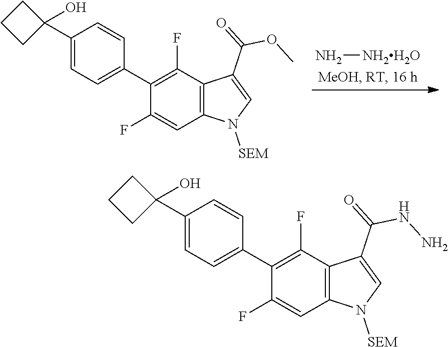

To a stirred solution of methyl 4,6-difluoro-5-(4-(1-hydroxycyclobutyl)phenyl)-1-((2-(trimethylsilyl)ethoxy)methyl)-1H-indole-3-carboxylate (1.0 g, 2.05 mmol, 1.0 eq) in MeOH (10 mL) at room temperature, was added NH₂—NH₂·H₂O (20 mL, 20 Vol) and stirred for 16 h. After completion of reaction by TLC, the reaction mixture was concentrated under reduced pressure to afford crude product. The crude product was triturated with 10% diethyl ether in n-pentane (50 mL), solid was filtered and dried under vacuum to afford 4,6-difluoro-5-(4-(1-hydroxycyclobutyl)phenyl)-1-((2-(trimethylsilyl)ethoxy)methyl)-1H-indole-3-carbohydrazide as off white solid (1.0 g, Yield: Crude). TLC system: EtOAc/Hexane (100:00), R_{f} value: ˜0.4; LCMS (m/z): 488.38 (M+H)⁺.

### Synthesis of 1-(4-(3-(5-(difluoromethyl)-1,3,4-oxadiazol-2-yl)-4,6-difluoro-1-((2-(trimethylsilyl)ethoxy)methyl)-1H-indol-5-yl)phenyl)cyclobutan-1-ol

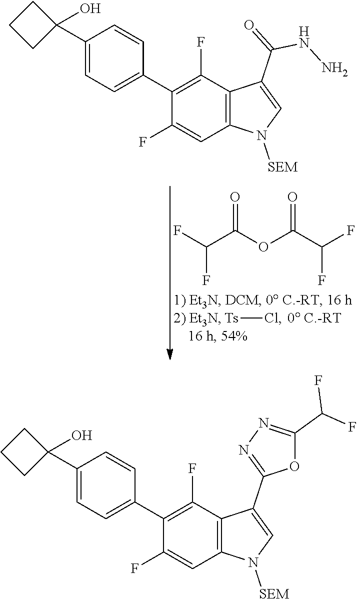

To a stirred solution of 4,6-difluoro-5-(4-(1-hydroxycyclobutyl)phenyl)-1-((2-(trimethylsilyl)ethoxy)methyl)-1H-indole-3-carbohydrazide (1.0 g, 2.05 mmol, 1.0 eq) in DCM (25 mL) at 0° C., was added Et₃N (621 mg, 6.15 mmol, 3.0 eq) and 2,2-difluoroacetic anhydride (714 mg, 4.11 mmol, 2.0 eq) drop wise and stirred at room temperature for 16 h. Later, added Et₃N (207 mg, 2.05 mmol, 1.0 eq) and Ts-Cl (1.17 g, 6.15 mmol, 3.0 eq) and stirred at room temperature for 16 h. After completion of reaction by TLC, the reaction mixture was quenched with water (50 mL) and extracted with DCM (3×30 mL). The combined organic layer was dried over Na₂SO₄, filtered and concentrated under reduced pressure to afford crude product. The crude product was purified by silica gel (100-200) column chromatography [elution with 20% EtOAc in petroleum ether] to afford 1-(4-(3-(5-(difluoromethyl)-1,3,4-oxadiazol-2-yl)-4,6-difluoro-1-((2-(trimethylsilyl)ethoxy)methyl)-1H-indol-5-yl)phenyl)cyclobutan-1-ol as a pale yellow gum (600 mg, Yield: 54%). TLC system: EtOAc/Hexane (30:70), R_{f} value: ˜0.5; LCMS (m/z): 548.45 (M+H)⁺.

### Synthesis of 1-(4-(3-(5-(difluoromethyl)-1,3,4-oxadiazol-2-yl)-4,6-difluoro-1H-indol-5-yl)phenyl)cyclobutan-1-ol

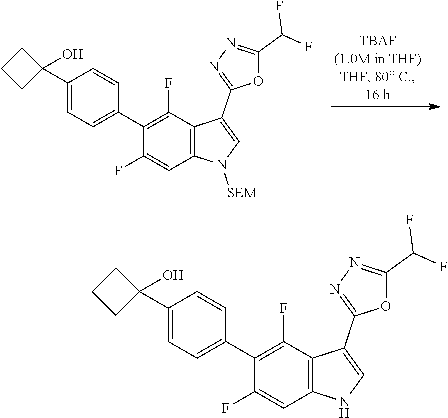 [SMILES: C[Si](C)(C)CCOCn1cc(-c2nnc(C(F)F)o2)c2c(F)c(-c3ccc(C4(O)CCC4)cc3)c(F)cc21 ; OC1(c2ccc(-c3c(F)cc4[nH]cc(-c5nnc(C(F)F)o5)c4c3F)cc2)CCC1]

To a stirred solution of 1-(4-(3-(5-(difluoromethyl)-1,3,4-oxadiazol-2-yl)-4,6-difluoro-1-((2-(trimethylsilyl)ethoxy)methyl)-1H-indol-5-yl)phenyl)cyclobutan-1-ol (4×50 mg, 0.36 mmol, 1.0 eq) in THF (10 Vol) at 0° C., was added TBAF (1.0 M in THF) (1.28 mL, 1.28 mmol, 3.5 eq) and stirred at 80° C. for 16 h. After completion of reaction by TLC, the reaction mixture was quenched with water (5 mL) and extracted with 10% MeOH in DCM (2×5 mL). The combined organic layer was dried over Na₂SO₄, filtered and concentrated under reduced pressure. The crude product was purified by reverse phase column chromatography [elution with 60% of ACN in 0.1% of FA in H₂O] followed by Prep-HPLC. The collected pure fractions were frozen and lyophilized to afford 1-(4-(3-(5-(difluoromethyl)-1,3,4-oxadiazol-2-yl)-4,6-difluoro-1H-indol-5-yl)phenyl)cyclobutan-1-ol as white solid (15 mg, Yield: 10%). TLC system: EtOAc/Hexane (50:50), R_{f} value: ˜0.4; LCMS (m/z): 416.21 (M+H)⁺; ¹H NMR (400 MHz, DMSO-d₆) δ: 12.56 (brs, 1H), 8.39 (s, 1H), 7.64-7.39 (m, 5H), 7.36 (d, J=9.6 Hz, 1H), 5.55 (s, 1H), 2.48-2.42 (m, 2H), 2.35-2.28 (m, 2H), 1.98-1.93 (m, 1H), 1.74-1.67 (m, 1H).

### SYNTHETIC EXAMPLE 43

### Synthesis of Compound I-69

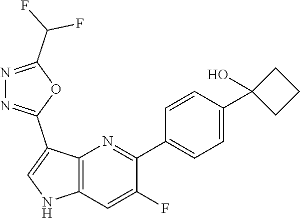 [SMILES: OC1(c2ccc(-c3nc4c(-c5nnc(C(F)F)o5)c[nH]c4cc3F)cc2)CCC1]

### Synthesis of 5-chloro-6-fluoro-1-((2-(trimethylsilyl)ethoxy)methyl)-1H-pyrrolo[3,2-b]pyridine-3-carbaldehyde

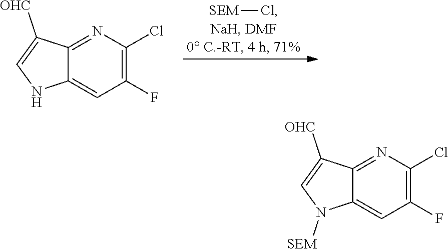 [SMILES: O=Cc1c[nH]c2cc(F)c(Cl)nc12 ; C[Si](C)(C)CCOCCl ; C[Si](C)(C)CCOCn1cc(C=O)c2nc(Cl)c(F)cc21]

To a stirred solution of 5-chloro-6-fluoro-1H-pyrrolo[3,2-b]pyridine-3-carbaldehyde (1.3 g, 6.53 mmol, 1.0 eq) in DMF (13 mL) at 0° C., was added NaH (523 mg, 13.06 mmol, 2.0 eq) and stirred for 30 min. Then SEM-Cl (2.73 g, 16.3 mmol, 2.5 eq) was added to reaction mixture and stirred at room temperature for 4 h. After completion of reaction by TLC, the reaction mixture was quenched with ice cold water (60 mL) and extracted with EtOAc (2×50 mL). The combined organic layer was dried over Na₂SO₄, filtered and concentrated under reduced pressure to afford crude product. The crude product was purified by silica gel [60-120] column chromatography [elution with 15% EtOAc in petroleum ether] to afford 5-chloro-6-fluoro-1-((2-(trimethylsilyl)ethoxy)methyl)-1H-pyrrolo[3,2-b]pyridine-3-carbaldehyde as a yellow solid (1.5 g, Yield: 71%). TLC system: EtOAc/Hexane (30:70), R_{f} value: ˜0.5; ¹H NMR (400 MHz, CDCl₃) δ: 10.37 (s, 1H), 8.09 (s, 1H), 7.74 (d, J=8.4 Hz, 1H), 5.54 (s, 2H), 3.54-3.50 (m, 2H), 0.96-0.92 (m, 2H), −0.01 (s, 9H).

### Synthesis of 5-chloro-6-fluoro-1-((2-(trimethylsilyl)ethoxy)methyl)-1H-pyrrolo[3,2-b]pyridine-3-carboxylic acid

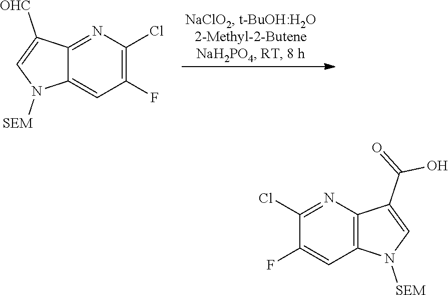 [SMILES: C[Si](C)(C)CCOCn1cc(C=O)c2nc(Cl)c(F)cc21 ; C[Si](C)(C)CCOCn1cc(C(=O)O)c2nc(Cl)c(F)cc21]

To a stirred solution of 5-chloro-6-fluoro-1-((2-(trimethylsilyl)ethoxy)methyl)-1H-pyrrolo[3,2-b]pyridine-3-carbaldehyde (1.5 g, 4.56 mmol, 1.0 eq) in t-BuOH (7.5 mL) and H₂O (7.5 mL) at room temperature, was added NaClO₂ (4.1 g, 45.6 mmol, 10 eq), NaH₂PO₄ (5.47 g, 45.6 mmol, 10 eq) followed by 2-methyl-2-butene (6.38 g, 91.2 mmol, 20 eq) and stirred for 8 h at room temperature. After completion of reaction by TLC, the reaction mixture was diluted with water (80 mL) and extracted with DCM (2×50 mL). The combined organic layer was dried over Na₂SO₄, filtered and concentrated under reduced pressure to afford 5-chloro-6-fluoro-1-((2-(trimethylsilyl)ethoxy)methyl)-1H-pyrrolo[3,2-b]pyridine-3-carboxylic acid as yellow solid (1.4 g, Yield: Crude). TLC system: EtOAc/Hexane (50:50), R_{f} value: ˜0.3; LCMS (m/z): 345.23 (M+H)⁺; ¹H NMR (400 MHz, CDCl₃) δ: 8.19 (s, 1H), 7.81 (d, J=8.0 Hz, 1H), 5.54 (s, 2H), 3.54-3.50 (m, 2H), 0.96-0.92 (m, 2H), −0.01 (s, 9H).

### Synthesis of methyl 5-chloro-6-fluoro-1-((2-(trimethylsilyl)ethoxy)methyl)-1H-pyrrolo[3,2-b]pyridine-3-carboxylate

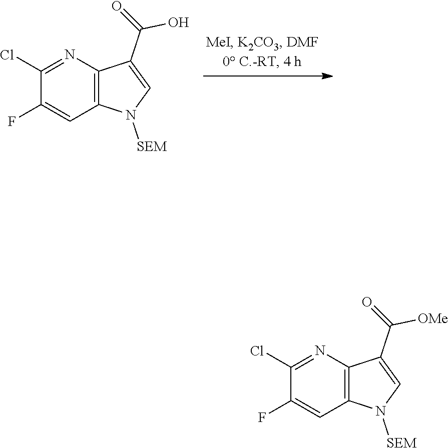

To a stirred solution of afford 5-chloro-6-fluoro-1-((2-(trimethylsilyl)ethoxy)methyl)-1H-pyrrolo[3,2-b]pyridine-3-carboxylic acid (1.4 g, 4.06 mmol, 1.0 eq) in DMF (21 mL) at 0° C., was added K₂CO₃ (1.12 g, 8.12 mmol, 2.0 eq) and MeI (1.15 g, 8.12 mmol, 2.0 eq) and stirred for 4 h at room temperature. After completion of reaction by TLC, the reaction mixture was diluted with water (30 mL) and extracted with EtOAc (2×20 mL). The combined organic layer was washed with brine solution (50 mL), dried over Na₂SO₄, filtered and concentrated under reduced pressure to afford methyl 5-chloro-6-fluoro-1-((2-(trimethylsilyl)ethoxy)methyl)-1H-pyrrolo[3,2-b]pyridine-3-carboxylate as yellow solid (1.5 g, Crude). TLC system: EtOAc/Hexane (30:70), R_{f} value: ˜0.5; LCMS (m/z): 359.30 (M+H)⁺.

### Synthesis of 5-chloro-6-fluoro-1-((2-(trimethylsilyl)ethoxy)methyl)-1H-pyrrolo[3,2-b]pyridine-3-carbohydrazide

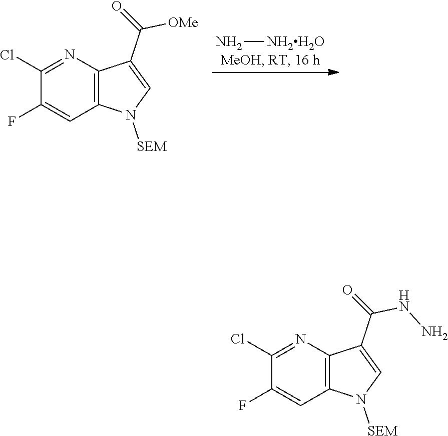

To a stirred solution of methyl 5-chloro-6-fluoro-1-((2-(trimethylsilyl)ethoxy)methyl)-1H-pyrrolo[3,2-b]pyridine-3-carboxylate (1.3 g, 3.63 mmol, 1.0 eq) in MeOH (13 mL) at room temperature, was added NH₂—NH₂·H₂O (65 mL, 50 Vol) and stirred for 16 h. After completion of reaction by TLC, the reaction mixture was filtered, washed with n-pentane (10 mL) and collected solid was dried under vacuum to afford 5-chloro-6-fluoro-1-((2-(trimethylsilyl)ethoxy)methyl)-1H-pyrrolo[3,2-b]pyridine-3-carbohydrazide as off white solid (1.0 g, Crude). TLC system: EtOAc/Hexane (100:00), R_{f} value: ˜0.3; LCMS (m/z): 359.26 (M+H)⁺.

### Synthesis of 2-(5-chloro-6-fluoro-1-((2-(trimethylsilyl)ethoxy)methyl)-1H-pyrrolo[3,2-b]pyridin-3-yl)-5-(difluoromethyl)-1,3,4-oxadiazole

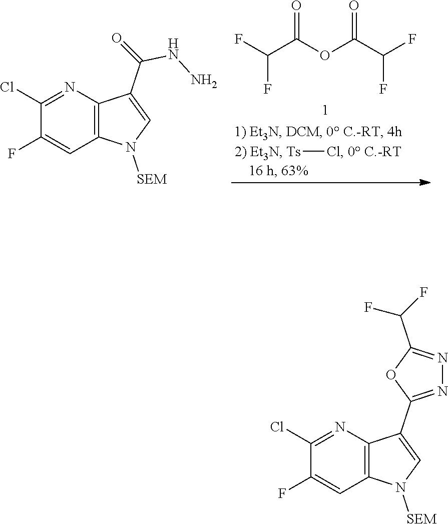

To a stirred solution of 5-chloro-6-fluoro-1-((2-(trimethylsilyl)ethoxy)methyl)-1H-pyrrolo[3,2-b]pyridine-3-carbohydrazide (1.0 g, 2.78 mmol, 1.0 eq) in DCM (25 mL) at 0° C., was added Et₃N (842 mg, 8.34 mmol, 3.0 eq) and 2,2-difluoroacetic anhydride (580 mg, 3.34 mmol, 1.2 eq) drop wise and stirred at room temperature for 4 h. Then added Et₃N (281 mg, 2.78 mmol, 1.0 eq) and Ts-Cl (1.58 g, 8.34 mmol, 3.0 eq) and stirred at room temperature for 16 h. After completion of reaction by TLC, the reaction mixture was quenched with water (50 mL) and extracted with DCM (2×50 mL). The combined organic layer was washed with saturated NaHCO₃ solution (50 mL), dried over Na₂SO₄, filtered and concentrated under reduced pressure. The crude product was purified by silica gel (100-200) column chromatography [elution with 15% EtOAc in petroleum ether] to afford 2-(5-chloro-6-fluoro-1-((2-(trimethylsilyl)ethoxy)methyl)-1H-pyrrolo[3,2-b]pyridin-3-yl)-5-(difluoromethyl)-1,3,4-oxadiazole as yellow gum (700 mg, Yield: 63%). TLC system: EtOAc/Hexane (30:70), R_{f} value: ˜0.5; LCMS (m/z): 419.24 (M+H)⁺; ¹H NMR (400 MHz, CDCl₃) δ: 8.19 (s, 1H), 7.74 (d, J=8.0 Hz, 1H), 6.96 (t, J=11.6 Hz, 1H), 5.54 (s, 2H), 3.54-348 (m, 2H), 0.94-0.89 (m, 2H), −0.02 (s, 9H).

### Synthesis of 1-(4-(3-(5-(difluoromethyl)-1,3,4-oxadiazol-2-yl)-6-fluoro-1-((2-(trimethylsilyl)ethoxy)methyl)-1H-pyrrolo[3,2-b]pyridin-5-yl)phenyl)cyclobutan-1-ol

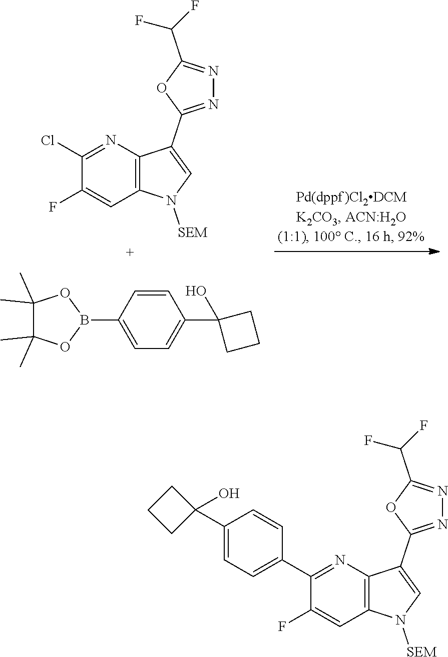 [SMILES: C[Si](C)(C)CCOCn1cc(-c2nnc(C(F)F)o2)c2nc(Cl)c(F)cc21 ; CC1(C)OB(c2ccc(C3(O)CCC3)cc2)OC1(C)C ; C[Si](C)(C)CCOCn1cc(-c2nnc(C(F)F)o2)c2nc(-c3ccc(C4(O)CCC4)cc3)c(F)cc21]

In a sealed tube to a degassed solution of 2-(5-chloro-6-fluoro-1-((2-(trimethylsilyl)ethoxy)methyl)-1H-pyrrolo[3,2-b]pyridin-3-yl)-5-(difluoromethyl)-1,3,4-oxadiazole (300 mg, 0.72 mmol, 1.0 eq) and 1-(4-(4,4,5,5-tetramethyl-1,3,2-dioxaborolan-2-yl)phenyl)cyclobutan-1-ol (392 mg, 1.43 mmol, 2.0 eq) in ACN:H₂O (3:1, 10 Vol) at room temperature, was added K₂CO₃ (248 mg, 1.80 mmol, 2.5 eq) followed by Pd(dppf)Cl₂·DCM (59 mg, 0.072 mmol, 0.1 eq) and stirred at 100° C. for 16 h. After completion of reaction by TLC, the reaction mixture was diluted with water (15 mL) and extracted with EtOAc (2×15 mL). The combined organic layer was dried over Na₂SO₄, filtered and concentrated under reduced pressure. The crude product was purified by silica gel [100-200] column chromatography [elution with 20% EtOAc in petroleum ether] to afford 6-fluoro-5-(4-(1-hydroxycyclobutyl)phenyl)-1-((2-(trimethylsilyl)ethoxy)methyl)-1H-pyrrolo[3,2-b]pyridine-3-carbonitrile as colorless gum (350 mg, Yield: 92%). TLC system: EtOAc/Hexane (30:50), R_{f} value: ˜0.3; LCMS (m/z): 531.39 (M+H)⁺.

### Synthesis of 1-(4-(3-(5-(difluoromethyl)-1,3,4-oxadiazol-2-yl)-6-fluoro-1H-pyrrolo[3,2-b]pyridin-5-yl)phenyl)cyclobutan-1-ol

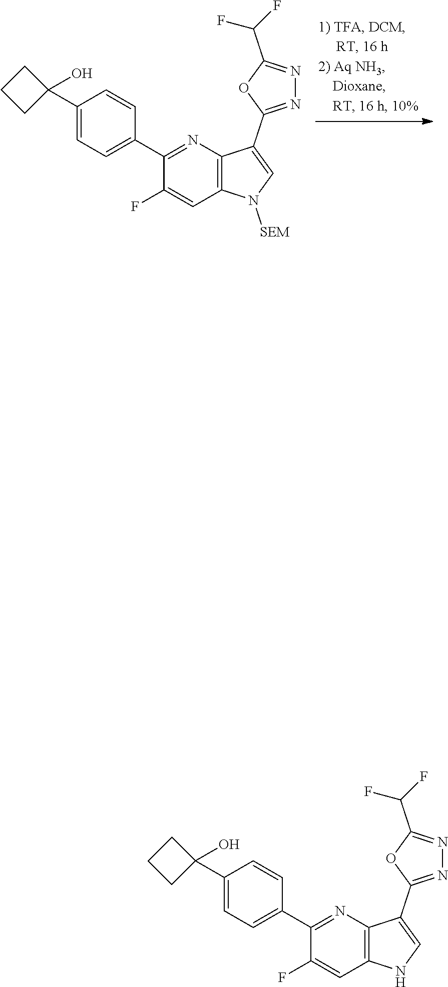 [SMILES: C[Si](C)(C)CCOCn1cc(-c2nnc(C(F)F)o2)c2nc(-c3ccc(C4(O)CCC4)cc3)c(F)cc21 ; OC1(c2ccc(-c3nc4c(-c5nnc(C(F)F)o5)c[nH]c4cc3F)cc2)CCC1]

To a stirred solution of 6-fluoro-5-(4-(1-hydroxycyclobutyl)phenyl)-1-((2-(trimethylsilyl)ethoxy)methyl)-1H-pyrrolo[3,2-b]pyridine-3-carbonitrile (350 mg, 0.64 mmol, 1.0 eq) in DCM (10 Vol) at 0° C., was added TFA (1.8 mL, 6 Vol) and stirred at room temperature for 16 h. After observing conversion, reaction mixture was concentrated, dissolved in 1,4 Dioxane (10 Vol) and added aq. NH₃, stirred for 16 h at room temperature. After completion of reaction by TLC, the reaction mixture was concentrated under reduced pressure. The crude product was purified by Prep-HPLC, collected fractions were freeze and lyophilized to afford 1-(4-(3-(5-(difluoromethyl)-1,3,4-oxadiazol-2-yl)-6-fluoro-1H-pyrrolo[3,2-b]pyridin-5-yl)phenyl)cyclobutan-1-ol as white solid (22 mg, Yield: 10%). TLC system: MeOH/DCM (10:90), R_{f} value: ˜0.2; LCMS (m/z): 401.25 (M+H)⁺; ¹H NMR (400 MHz, DMSO-d₆) δ: 12.47 (brs, 1H), 8.61 (s, 1H), 7.98-7.93 (m, 3H), 7.70-7.45 (m, 3H), 5.59 (s, 1H), 2.45-2.42 (m, 2H), 2.36-2.28 (m, 2H), 1.97-1.94 (m, 1H), 1.72-1.69 (m, 1H).

### SYNTHETIC EXAMPLE 44

### Synthesis of Compound I-71

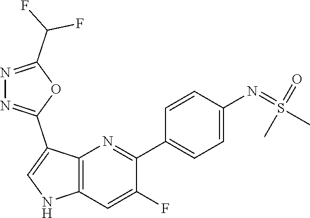 [SMILES: CS(C)(=O)=Nc1ccc(-c2nc3c(-c4nnc(C(F)F)o4)c[nH]c3cc2F)cc1]

### Synthesis of ((4-(3-(5-(difluoromethyl)-1,3,4-oxadiazol-2-yl)-6-fluoro-1-((2-(trimethylsilyl)ethoxy)methyl)-1H-pyrrolo[3,2-b]pyridin-5-yl)phenyl) imino) dimethyl-16-sulfanone

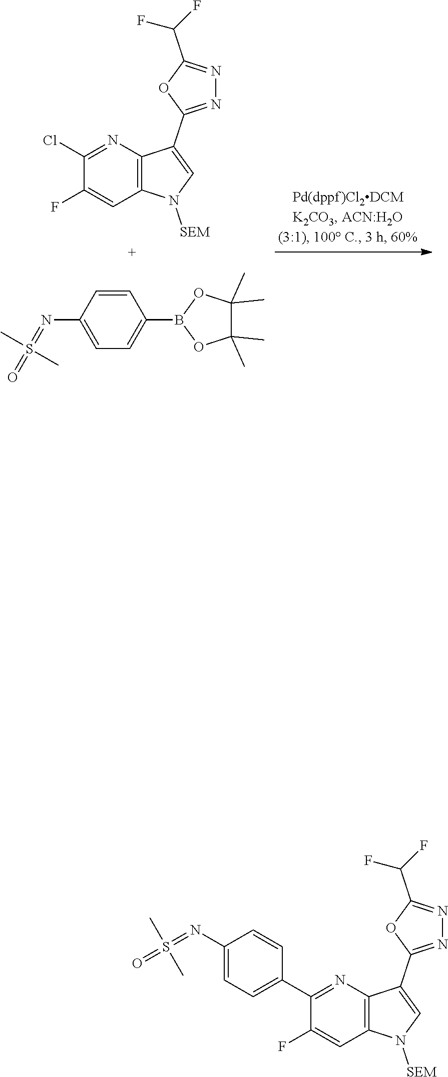

In a sealed tube to a degassed solution of 2-(5-chloro-6-fluoro-1-((2-(trimethylsilyl)ethoxy)methyl)-1H-pyrrolo[3,2-b]pyridin-3-yl)-5-(difluoromethyl)-1,3,4-oxadiazole (800 mg, 1.91 mmol, 1.0 eq) and dimethyl ((4-(4,4,5,5-tetramethyl-1,3,2-dioxaborolan-2-yl)phenyl) imino)-16-sulfanon (1.13 g, 3.82 mmol, 2.0 eq) in ACN:H₂O (3:1, 20 Vol) at room temperature, was added K₂CO₃ (660 mg, 4.77 mmol, 2.5 eq) followed by Pd(dppf)Cl₂·DCM (156 mg, 0.19 mmol, 0.1 eq) and stirred at 100° C. for 3 h. After completion of reaction by TLC, the reaction mixture was diluted with water (40 mL) and extracted with EtOAc (2×30 mL). The combined organic layer was dried over Na₂SO₄, filtered and concentrated under reduced pressure. The crude product was purified by silica gel [100-200] column chromatography [elution with 20% EtOAc in petroleum ether] to afford ((4-(3-(5-(difluoromethyl)-1,3,4-oxadiazol-2-yl)-6-fluoro-1-((2-(trimethylsilyl)ethoxy)methyl)-1H-pyrrolo[3,2-b]pyridin-5-yl)phenyl) imino) dimethyl-16-sulfanone as an off white solid (600 mg, Yield: 60%). TLC system: EtOAc/Hexane (100:00), R_{f} value: ˜0.5; LCMS (m/z): 552.44 (M+H)⁺.

### Synthesis of ((4-(3-(5-(difluoromethyl)-1,3,4-oxadiazol-2-yl)-6-fluoro-1H-pyrrolo[3,2-b]pyridin-5-yl)phenyl) imino) dimethyl-16-sulfanone

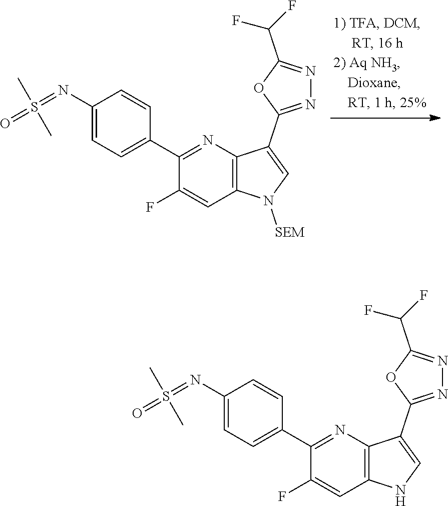 [SMILES: C[Si](C)(C)CCOCn1cc(-c2nnc(C(F)F)o2)c2nc(-c3ccc(N=S(C)(C)=O)cc3)c(F)cc21 ; CS(C)(=O)=Nc1ccc(-c2nc3c(-c4nnc(C(F)F)o4)c[nH]c3cc2F)cc1]

To a stirred solution of ((4-(3-(5-(difluoromethyl)-1,3,4-oxadiazol-2-yl)-6-fluoro-1-((2-(trimethylsilyl)ethoxy)methyl)-1H-pyrrolo[3,2-b]pyridin-5-yl)phenyl) imino) dimethyl-16-sulfanone (150 mg, 0.27 mmol, 1.0 eq) in DCM (10 Vol) at 0° C., was added TFA (1.0 mL, 6 Vol) and stirred at room temperature for 16 h. After observing conversion, reaction mixture was concentrated, dissolved in 1,4 dioxane (10 Vol) and added aq. NH₃ (0.3 mL, 2 Vol), stirred for 1 h at room temperature. After completion of reaction by TLC, reaction mixture was concentrated under reduced pressure and purified by Prep-HPLC. The collected fractions were frozen and lyophilized to afford ((4-(3-(5-(difluoromethyl)-1,3,4-oxadiazol-2-yl)-6-fluoro-1H-pyrrolo[3,2-b]pyridin-5-yl)phenyl) imino) dimethyl-16-sulfanone as white solid (28 mg, Yield: 25%). TLC system: MeOH/DCM (10:90), R_{f} value: ˜0.2; LCMS (m/z): 422.21 (M+H)⁺; ¹H NMR (400 MHz, DMSO-d₆) δ: 12.40 (brs, 1H), 8.56 (s, 1H), 7.92 (d, J=12.0 Hz, 1H), 7.86 (dd, J=1.6 Hz, 8.4 Hz, 2H), 7.57 (t, J=51.6 Hz, 1H), 7.07 (d, J=8.8 Hz, 2H), 3.28 (s, 6H).

### SYNTHETIC EXAMPLE 45

### Synthesis of Compound II-2

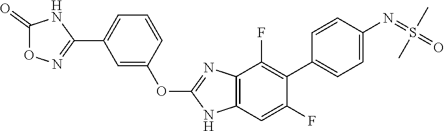 [SMILES: CS(C)(=O)=Nc1ccc(-c2c(F)cc3[nH]c(Oc4cccc(-c5noc(=O)[nH]5)c4)nc3c2F)cc1]

### Synthesis of N-(4-bromo-3,5-difluoro-2-nitrophenyl)acetamide

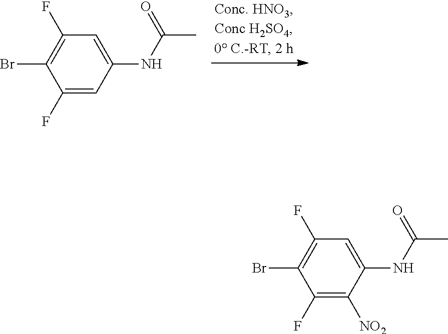 [SMILES: CC(=O)Nc1cc(F)c(Br)c(F)c1 ; CC(=O)Nc1cc(F)c(Br)c(F)c1[N+](=O)[O-]]

To a stirred solution of N-(4-bromo-3,5-difluorophenyl)acetamide (20 g, 80.3 mol, 1.0 eq) in concentrated HNO₃ (28 mL, 1.4 Vol) at 0° C., was added concentrated H₂SO₄ (28 mL, 1.4 Vol) dropwise. After 10 min, the reaction mixture was allowed to room temperature and stirred for another 2 h. After completion of reaction by TLC, the reaction mixture poured into ice cold water, precipitated solid was filtered and washed with n-pentane to afford N-(4-bromo-3,5-difluoro-2-nitrophenyl)acetamide as light-yellow color solid (15 g, 63%). TLC system: EtOAc/Hexane (30:70), R_{f} value: ^{˜}0.4; LCMS (m/z): 293.02 (M−H)⁺; ¹H NMR (400 MHz, CDCl₃) δ: 9.25 (brs, 1H), 8.39 (dd, J=8.4 Hz, 2.4 Hz, 1H), 2.27 (s, 3H).

### Synthesis of 4-bromo-3,5-difluoro-2-nitroaniline

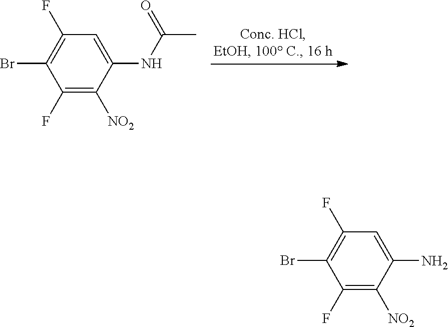 [SMILES: CC(=O)Nc1cc(F)c(Br)c(F)c1[N+](=O)[O-] ; Nc1cc(F)c(Br)c(F)c1[N+](=O)[O-]]

To a stirred of solution of N-(4-bromo-3,5-difluoro-2-nitrophenyl)acetamide (15 g, 51.0 mmol, 1.0 eq) in EtOH (150 mL, 10 Vol) at 0° C., was added Conc. HCl (75 mL, 5 Vol) drop-wise and reaction mixture was stirred at 100° C. for 16 h. After completion of reaction by TLC, the reaction mixture cooled to room temperature and evaporated under reduced pressure. Obtained residue was diluted with EtOAc (500 mL), washed with aq NaHCO₃ solution, dried over Na₂SO₄ and concentrated under reduced pressure to afford 4-bromo-3,5-difluoro-2-nitroaniline as yellow color solid (11 g, yield: 85%). TLC system: EtOAc/Hexane (20:80), R_{f} value: ^{˜}0.2; LCMS (m/z): 250.94 (M−H)⁺; ¹H NMR (400 MHz, CDCl₃) δ: 6.44 (dd, J=7.6 Hz, 2.0 Hz, 1H), 5.86 (brs, 2H).

### Synthesis of 4-bromo-3,5-difluorobenzene-1,2-diamine

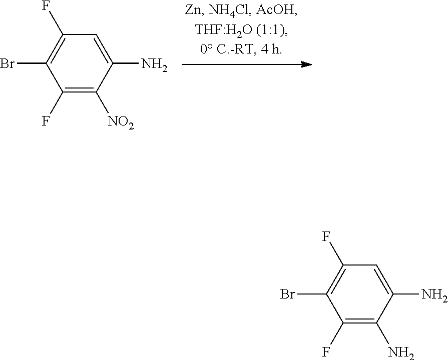 [SMILES: Nc1cc(F)c(Br)c(F)c1[N+](=O)[O-] ; Nc1cc(F)c(Br)c(F)c1N]

To a stirred solution of 4-bromo-3,5-difluoro-2-nitroaniline (11 g, 43.6 mmol, 1.0 eq) and NH₄Cl (9.4 g, 174.6 mmol, 4 eq) in THF:H₂O (1:1) (220 mL, 20 Vol) at 0° C., was added Zn dust (11.4 g, 174.6 mmol, 4 eq), acetic acid (3.3 mL, 0.3 Vol). The reaction mixture was stirred at room temperature for 4 h. After completion of reaction by TLC, the reaction mixture was filtered through a pad of diatomaceous earth (e.g., Celite®), and washed with 10% MeOH/DCM (200 mL). Filtrate was concentrated under reduced pressure to afford 4-bromo-3,5-difluorobenzene-1,2-diamine as a brown solid (8.5 g, yield: 87%). TLC system: EtOAc/Hexane (50:50), R_{f} value: ˜0.1; LCMS (m/z): 223.00 (M+H)⁺.

### Synthesis of 5-bromo-4,6-difluoro-1,3-dihydro-2H-benzo[d]imidazol-2-one

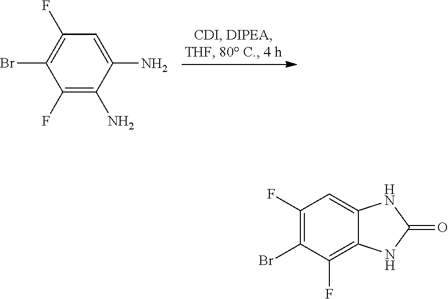 [SMILES: Nc1cc(F)c(Br)c(F)c1N ; O=c1[nH]c2cc(F)c(Br)c(F)c2[nH]1]

To a stirred solution of 4-bromo-3,5-difluorobenzene-1,2-diamine (8.5 g, 38.2 mmol, 1.0 eq) in THF (170 mL, 20 Vol) at room temperature, was added DIPEA (23.2 mL, 134.8 mmol, 3.5 eq), CDI (21.7 g, 134.8 mmol, 3.5 eq) and the reaction stirred at 80° C. for 4 h. After completion of reaction by TLC, the reaction mixture was diluted with ice cold water (70 mL) and acidified with 2N aq. HCl. Precipitated solid was filtered, washed with diethyl ether to provide 5-bromo-4,6-difluoro-1,3-dihydro-2H-benzo[d]imidazol-2-one as brown solid (7.8 g, crude). TLC system: EtOAc (100%), R_{f} value: ˜0.1; LCMS (m/z): 249.0 (M+H)⁺.

### Synthesis of 6-bromo-2-chloro-5,7-difluoro-1H-benzo[d]imidazole

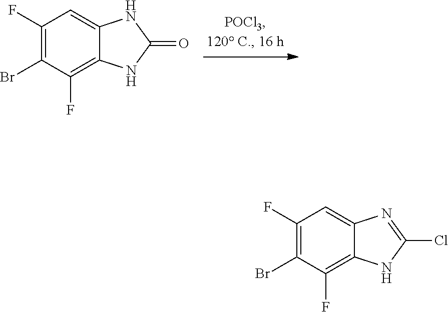 [SMILES: O=c1[nH]c2cc(F)c(Br)c(F)c2[nH]1 ; Fc1cc2nc(Cl)[nH]c2c(F)c1Br]

A solution of 5-bromo-4,6-difluoro-1,3-dihydro-2H-benzo[d]imidazol-2-one (7.8 g, 31.4 mmol, 1.0 eq) in POCl₃ (117 mL, 15 Vol) was stirred at 120° C. for 16 h. After completion of reaction by TLC, reaction mixture cooled to room temperature, evaporated under reduced pressure. Obtained residue quenched with aq. NaHCO₃ solution and extracted with EtOAc. Organic layer was dried over Na₂SO₄, concentrated and washed with n-pentane afforded 6-bromo-2-chloro-5,7-difluoro-1H-benzo[d]imidazole as light green color solid (8 g, yield: 79% in 2 steps). TLC system: EtOAc/Hexane (50:50), R_{f} value: 0.2; LCMS (m/z): 266.98 (M+H)⁺; ¹H NMR (400 MHz, CDCl₃) δ: 7.18-7.16 (m, 1H).

### Synthesis of 6-bromo-2-chloro-5,7-difluoro-1-((2-(trimethylsilyl)ethoxy)methyl)-1H-benzo[d]imidazole

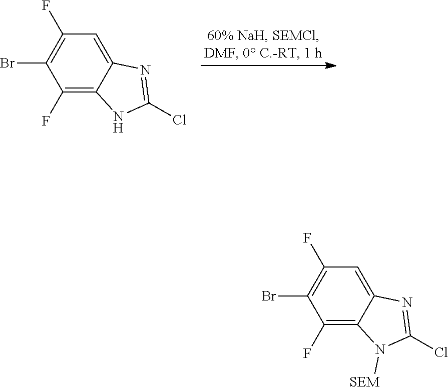 [SMILES: Fc1cc2nc(Cl)[nH]c2c(F)c1Br ; C[Si](C)(C)CCOCn1c(Cl)nc2cc(F)c(Br)c(F)c21]

To a stirred solution of 6-bromo-2-chloro-5,7-difluoro-1H-benzo[d]imidazole 3.9 g, 14.6 mmol, 1.0 eq) in DMF (39 mL, 10 Vol) at 0° C., was added 60% NaH (704 mg, 29.3 mmol, 2.0 eq) and stirred for 30 min. Later, SEM-Cl (3.86 mL, 21.9 mmol, 1.5 eq) was added and reaction mixture was allowed to room temperature and stirred for 1 h. After completion of reaction by TLC, the reaction mixture was diluted with ice cold water (80 mL) and extracted with EtOAc (2×300 mL). The combined organic layer was dried over Na₂SO₄, filtered and concentrated under reduced pressure to afford crude product. The crude product was purified by silica gel (100-200 mesh) column chromatography [elution with 10% EtOAc in Hexane] to afford 6-bromo-2-chloro-5,7-difluoro-1-((2-(trimethylsilyl)ethoxy)methyl)-1H-benzo[d]imidazole as colorless solid (1.7 g, Yield: 29%). TLC system: EtOAc/Hexane (10:90), R_{f} value: ˜0.3; LCMS (m/z): 397.13 (M+H)⁺.

### Synthesis of (Z)-N′,3-dihydroxybenzimidamide

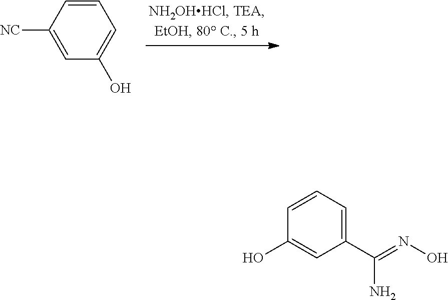 [SMILES: N#Cc1cccc(O)c1 ; N/C(=N\O)c1cccc(O)c1]

To a stirred solution of 3-hydroxybenzonitrile (5 g, 41.9 mmol, 1.0 eq) in EtOH (175 mL, 35 Vol) at room temperature, was added TEA (17 mL, 125.9 mmol, 3.0 eq), NH₂OH·HCl (5.8 g, 83.9 mmol, 2.0 eq) and stirred at 80° C. for 5 h. After completion of reaction by TLC, the reaction mixture evaporated under reduced pressure to afford (Z)-N′,3-dihydroxybenzimidamide a brown gummy solid (6.0 g, crude). TLC system: EtOAc/Hexane (50:50), R_{f} value: ˜0.1; LCMS (m/z): 153.10 (M+H)⁺.

### Synthesis of 3-(3-hydroxyphenyl)-1,2,4-oxadiazol-5(4H)-one

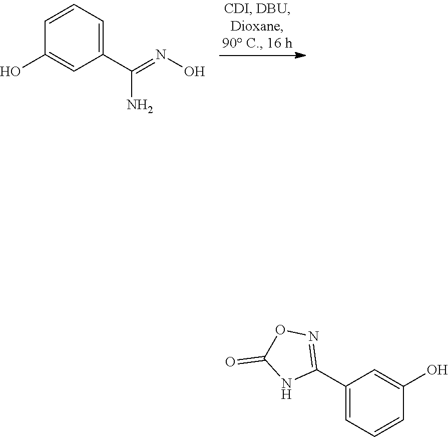 [SMILES: N/C(=N\O)c1cccc(O)c1 ; O=c1[nH]c(-c2cccc(O)c2)no1]

To a stirred solution of (Z)-N′,3-dihydroxybenzimidamide (3.0 g, 19.7 mmol, 1.0 eq) in dioxane (45 mL, 15 Vol) at room temperature, was added DBU (3.2 g, 21.7 mmol, 1.1 eq), CDI (3.5 g, 21.7 mmol, 1.1 eq) and stirred at 90° C. for 16 h. After completion of reaction by TLC, reaction mixture evaporated under reduced pressure, acidified with 2N aq. HCl and extracted with EtOAc (2×150 mL). The combined organic layer was dried over Na₂SO₄ and concentrated under reduced pressure to afford 3-(3-hydroxyphenyl)-1,2,4-oxadiazol-5(4H)-one as a brown solid (1.5 g, yield: 40% in 2 steps). TLC system: EtOAc (100%), R_{f} value: ˜0.2; LCMS (m/z): 179.02 (M+H)⁺.

### Synthesis of 3-(3-((6-bromo-5,7-difluoro-1-((2-(trimethylsilyl)ethoxy)methyl)-1H-benzo[d]imidazol-2-yl)oxy)phenyl)-1,2,4-oxadiazol-5(4H)-one

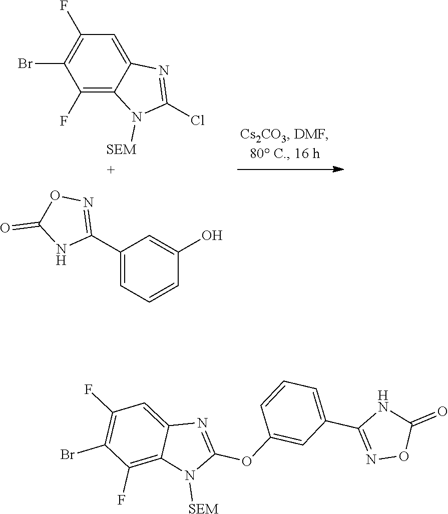 [SMILES: C[Si](C)(C)CCOCn1c(Cl)nc2cc(F)c(Br)c(F)c21 ; O=c1[nH]c(-c2cccc(O)c2)no1 ; C[Si](C)(C)CCOCn1c(Oc2cccc(-c3noc(=O)[nH]3)c2)nc2cc(F)c(Br)c(F)c21]

To a stirred solution of 6-bromo-2-chloro-5,7-difluoro-1-((2-(trimethylsilyl)ethoxy)methyl)-1H-benzo[d]imidazole (560 mg, 1.41 mmol, 1.0 eq) and 3-(3-hydroxyphenyl)-1,2,4-oxadiazol-5(4H)-one (326.4 mg, 1.83 mmol, 1.3 eq) in DMF (5.6 mL, 10 Vol) at room temperature, was added Cs₂CO₃ (919.6 mg, 2.82 mmol, 2.0 eq) and stirred at 80° C. for 16 h. After completion of reaction by TLC, the reaction mixture was diluted with water (30 mL) and extracted with EtOAc (2×80 mL). The combined organic layer was dried over Na₂SO₄, filtered and concentrated under reduced pressure to afford crude product. Purified by Reverse phase column, collected fractions were evaporated under reduced pressure to afford 3-(3-((6-bromo-5,7-difluoro-1-((2-(trimethylsilyl)ethoxy)methyl)-1H-benzo[d]imidazol-2-yl)oxy)phenyl)-1,2,4-oxadiazol-5(4H)-one as colorless gummy liquid (560 mg, Yield: 74%). TLC system: EtOAc/Hexane (50:50), R_{f} value: ˜0.2; LCMS (m/z): 537.16 (M−H)⁺.

### Synthesis of 3-(3-((5-(4-((dimethyl(oxo)-16-sulfaneylidene)amino)phenyl)-4,6-difluoro-1-((2-(trimethylsilyl)ethoxy)methyl)-1H-benzo[d]imidazol-2-yl)oxy)phenyl)-1,2,4-oxadiazol-5(4H)-one

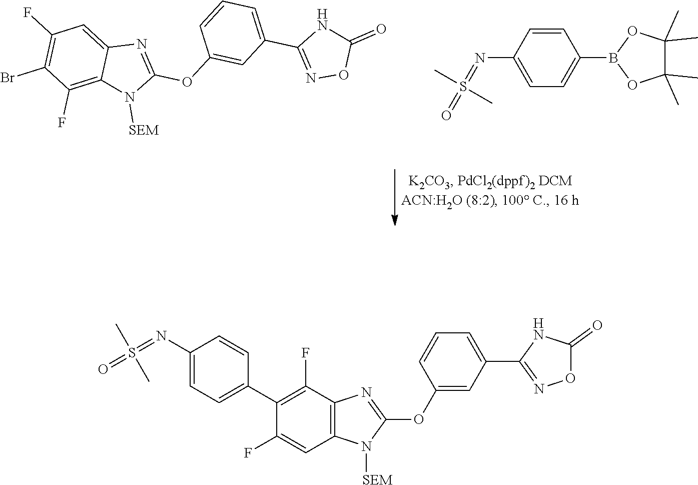

In a sealed tube to a degassed solution of 3-(3-((6-bromo-5,7-difluoro-1-((2-(trimethylsilyl)ethoxy)methyl)-1H-benzo[d]imidazol-2-yl)oxy)phenyl)-1,2,4-oxadiazol-5(4H)-one (560 mg, 1.04 mmol, 1.0 eq) and dimethyl ((4-(4,4,5,5-tetramethyl-1,3,2-dioxaborolan-2-yl)phenyl) imino)-16-sulfanone (768 mg, 2.60 mmol, 2.5 eq) in ACN:H₂O (8:2) (11.2 mL, 20 Vol) at room temperature, was added K₂CO₃ (431 mg, 3.12 mmol, 3.0 eq) followed by PdCl₂(dppf)·DCM (85 mg, 0.10 mmol, 0.1 eq) and stirred at 100° C. for 16 h. After completion of reaction by TLC, the reaction mixture was diluted with water (30 mL) and extracted with EtOAc (2× 80 mL). The combined organic layer was dried over Na₂SO₄, filtered and concentrated under reduced pressure. Obtained crude product was purified by reverse phase column and collected fractions were evaporated under reduced pressure to afford 3-(3-((5-(4-((dimethyl(oxo)-16-sulfaneylidene)amino)phenyl)-4,6-difluoro-1-((2-(trimethylsilyl)ethoxy)methyl)-1H-benzo[d]imidazol-2-yl)oxy)phenyl)-1,2,4-oxadiazol-5(4H)-one as brown solid (350 mg, Yield: 54%). TLC system: EtOAc (100%), R_{f} value: ˜0.2; LCMS (m/z): 628.41 (M+H)⁺.

### Synthesis of 3-(3-((5-(4-((dimethyl(oxo)-16-sulfaneylidene)amino)phenyl)-4,6-difluoro-1-(hydroxymethyl)-1H-benzo[d]imidazol-2-yl)oxy)phenyl)-1,2,4-oxadiazol-5(4H)-one

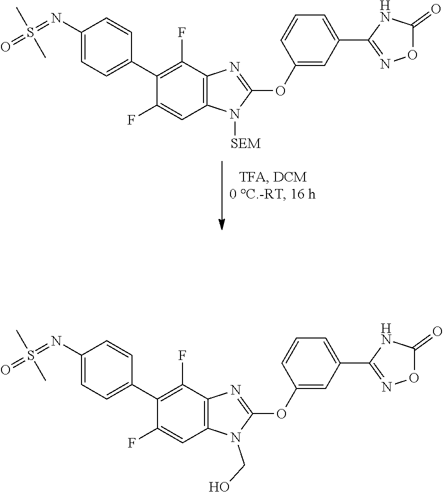 [SMILES: C[Si](C)(C)CCOCn1c(Oc2cccc(-c3noc(=O)[nH]3)c2)nc2c(F)c(-c3ccc(N=S(C)(C)=O)cc3)c(F)cc21 ; CS(C)(=O)=Nc1ccc(-c2c(F)cc3c(nc(Oc4cccc(-c5noc(=O)[nH]5)c4)n3CO)c2F)cc1]

To a stirred solution 3-(3-((5-(4-((dimethyl(oxo)-16-sulfaneylidene)amino)phenyl)-4,6-difluoro-1H-benzo[d]imidazol-2-yl)oxy)phenyl)-1,2,4-oxadiazol-5(4H)-one (350 mg, 0.55 mmol, 1.0 eq) in DCM (7 mL, 20 Vol) at 0° C., was added TFA (7 mL, 10 Vol) and stirred at room temperature for 16 h. After completion of reaction by TLC, the reaction mixture concentrated under reduced pressure and washed with ether (18 mL) to afford 3-(3-((5-(4-((dimethyl(oxo)-16-sulfaneylidene)amino)phenyl)-4,6-difluoro-1-(hydroxymethyl)-1H-benzo[d]imidazol-2-yl)oxy)phenyl)-1,2,4-oxadiazol-5(4H)-one as a colorless solid (200 mg, crude). TLC system: 10% MeOH/DCM (10:90), R_{f} value: ˜0.2; LCMS (m/z): 528.26 (M+H)⁺.

### Synthesis of 3-(3-((5-(4-((dimethyl(oxo)-16-sulfaneylidene)amino)phenyl)-4,6-difluoro-1H-benzo[d]imidazol-2-yl)oxy)phenyl)-1,2,4-oxadiazol-5(4H)-one

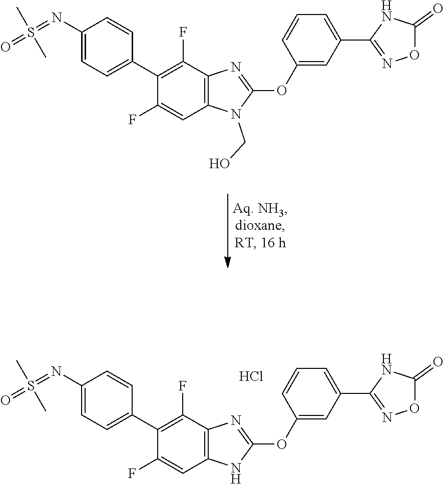 [SMILES: CS(C)(=O)=Nc1ccc(-c2c(F)cc3c(nc(Oc4cccc(-c5noc(=O)[nH]5)c4)n3CO)c2F)cc1 ; CS(C)(=O)=Nc1ccc(-c2c(F)cc3[nH]c(Oc4cccc(-c5noc(=O)[nH]5)c4)nc3c2F)cc1 ; Cl]

To a stirred solution of 3-(3-((5-(4-((dimethyl(oxo)-16-sulfaneylidene)amino)phenyl)-4,6-difluoro-1-(hydroxymethyl)-1H-benzo[d]imidazol-2-yl)oxy)phenyl)-1,2,4-oxadiazol-5(4H)-one (200 mg cr, 0.38 mmol, 1.0 eq) in Dioxane (4 mL, 20 Vol), was added aq. NH₃ (2 mL, 10 Vol) and stirred at room temperature for 16 h. After completion of reaction by TLC, the reaction mixture was concentrated under reduced pressure collected crude was dissolved in water (20 mL), the resulting solid was filtered, washed with ether (20 mL) followed by Prep-HPLC purification provided 08133 with clean NMR. To remove acetonitrile material was treated with 2N aq. HCl and lyophilized to afford 3-(3-((5-(4-((dimethyl(oxo)-16-sulfaneylidene)amino)phenyl)-4,6-difluoro-1H-benzo[d]imidazol-2-yl)oxy)phenyl)-1,2,4-oxadiazol-5(4H)-one HCl salt as brown solid (13.2 mg, Yield: 7%). TLC system: 10% MeOH/DCM (10:90), R_{f} value: ˜0.2; LCMS (m/z): 498.21 (M+H)⁺; ¹H NMR (400 MHz, DMSO-d₆) δ: 13.07 (s, 1H), 7.84-7.83 (m, 1H), 7.77-7.75 (m, 1H), 7.72-7.70 (m, 2H), 7.26-7.21 (m, 3H), 7.04-7.02 (m, 2H), 3.2 (s, 6H).

Prep-HPLC condition: SUNFIRE C-18 (250*19.0) mm 5mic, A: MP-(A): 0.2% FA in Water MP-(B): ACN:Water (80:20), Time: 0/40,15/60,20/95; diluent: Water/ACN/THF, Flow Rate: 13 ml/min.

### SYNTHETIC EXAMPLE 46

### Synthesis of Compound II-3

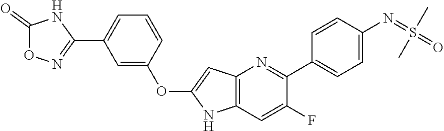 [SMILES: CS(C)(=O)=Nc1ccc(-c2nc3cc(Oc4cccc(-c5noc(=O)[nH]5)c4)[nH]c3cc2F)cc1]

### Synthesis of diethyl 2-(5-fluoro-3-nitropyridin-2-yl) malonate

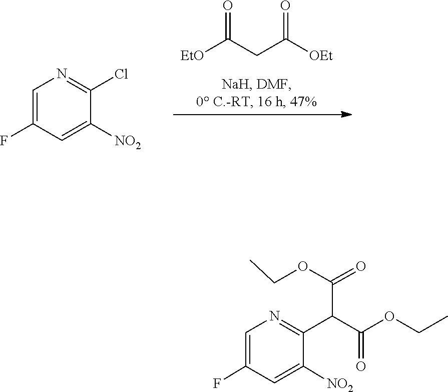 [SMILES: CCOC(=O)CC(=O)OCC ; O=[N+]([O-])c1cc(F)cnc1Cl ; CCOC(=O)C(C(=O)OCC)c1ncc(F)cc1[N+](=O)[O-]]

To a stirred solution of 2-chloro-5-fluoro-3-nitropyridine (15 g, 85.2 mmol, 1.0 eq) and diethyl malonate (25 mL, 89.5 mmol, 1.05 eq) in DMF (20 mL) at 0° C., was added 60% NaH (4.09 g, 170.4 mmol, 2.0 eq) portion wise and reaction mixture was allowed to room temperature and stirred for 16 h. After completion of reaction by TLC, the reaction mixture was diluted with ice cold water (100 mL) and extracted with EtOAc (2×200 mL). The combined organic layer was dried over Na₂SO₄, filtered and concentrated under reduced pressure. The crude product was purified by silica gel (60-120 mesh) column chromatography [elution with 15% EtOAc in Hexane] to afford diethyl 2-(5-fluoro-3-nitropyridin-2-yl) malonate as a brown liquid (12.1 g, Yield: 47%). TLC system: EtOAc/Hexane (20:80), R_{f} value: ˜0.2; ¹H NMR (400 MHz, CDCl₃) δ: 8.72 (d, J=2.8 Hz, 1H), 8.24 (dd, J=7.6 Hz, 2.8 Hz, 1H), 5.49 (s, 1H), 4.33-4.27 (m, 4H), 1.30 (t, J=7.4 Hz, 6H).

### Synthesis of ethyl 2-(5-fluoro-3-nitropyridin-2-yl)acetate

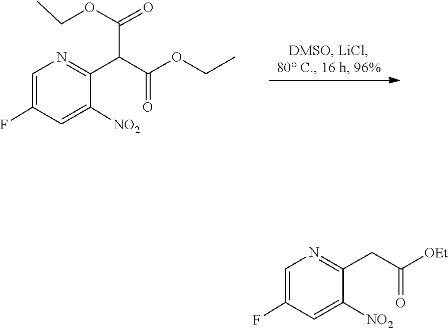 [SMILES: CCOC(=O)C(C(=O)OCC)c1ncc(F)cc1[N+](=O)[O-] ; CCOC(=O)Cc1ncc(F)cc1[N+](=O)[O-]]

To a stirred solution of diethyl 2-(5-fluoro-3-nitropyridin-2-yl) malonate (15 g, 50.0 mmol, 1 eq) in DMSO:H₂O (1:1) (150 mL, 10 Vol) at room temperature, was added LiCl (32 g, 750 mmol, 15 eq) portion wise and stirred at 80° C. for 16 h. After completion of reaction by TLC, the reaction mixture was diluted with ice cold water (100 mL) and extracted with EtOAc (2×200 mL). The combined organic layer was dried over Na₂SO₄, filtered and concentrated under reduced pressure. The crude product was purified by silica gel (60-120 mesh) column chromatography [elution with 5-10% EtOAc in Hexane] to afford ethyl 2-(5-fluoro-3-nitropyridin-2-yl)acetate as pale-yellow liquid (11 g, Yield: 96%). TLC system: EtOAc/Hexane (10:90), R_{f} value: ˜0.6; LCMS (m/z): 229.00 (M+H)⁺. ¹H NMR (400 MHz, CDCl₃) δ: 8.70 (d, J=2.4 Hz, 1H), 8.20 (dd, J=7.6 Hz, 2.8 Hz, 1H), 4.32 (s, 2H), 4.19 (q, J=7.2 Hz, 2H), 1.27 (t, J=7.2 Hz, 3H).

### Synthesis of ethyl 2-(3-amino-5-fluoropyridin-2-yl)acetate

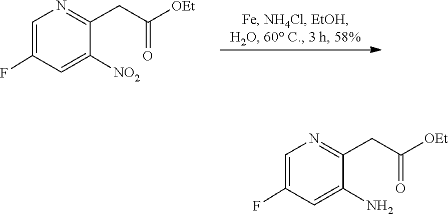 [SMILES: CCOC(=O)Cc1ncc(F)cc1[N+](=O)[O-] ; CCOC(=O)Cc1ncc(F)cc1N]

To a stirred solution of ethyl 2-(5-fluoro-3-nitropyridin-2-yl)acetate (11 g, 48.2 mmol, 1 eq) in EtOH:H₂O (2:1) (10 mL) at room temperature, was added Fe (10.7 g, 193 mmol, 4 eq) and NH₄Cl (10.3 g, 193 mmol, 5 eq) and heated to 80° C., stirred for 3 h. After completion of reaction by TLC, reaction mixture was filtered through a pad of diatomaceous earth (e.g., Celite®), and washed with EtOAc (30 mL). Filtrate was concentrated and purified by silica gel (60-120 mesh) column chromatography [elution with 5-10% EtOAc in Hexane] to afford ethyl 2-(3-amino-5-fluoropyridin-2-yl)acetate as brown solid (5.6 g, Yield: 58%). TLC system: EtOAc/Hexane (20:80), R_{f} value: ˜0.2. LCMS (m/z): 199.14 (M+H)⁺.

### Synthesis of ethyl 2-(3-amino-6-bromo-5-fluoropyridin-2-yl)acetate

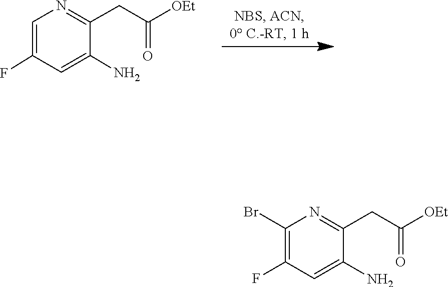 [SMILES: CCOC(=O)Cc1ncc(F)cc1N ; CCOC(=O)Cc1nc(Br)c(F)cc1N]

To a stirred solution of ethyl 2-(3-amino-5-fluoropyridin-2-yl)acetate (5 g, 25.2 mmol, 1.0 eq) in ACN (100 mL) at 0° C. was added NBS (3.59 g, 20.2 mmol, 0.8 eq) and stirred room temperature for 1 h. After completion of reaction by TLC, reaction mixture was concentrated and diluted with ice cold water (100 mL), precipitated solid was filtered, washed with diethyl ether (20 mL), pentane (20 mL) and dried under vacuum to afford ethyl 2-(3-amino-6-bromo-5-fluoropyridin-2-yl)acetate as a brown solid (6 g, crude). TLC system: EtOAc/petroleum ether (20:80), R_{f} value: ˜0.6; LCMS (m/z): 277.03 (M+H)⁺.

### Synthesis of 5-bromo-6-fluoro-1H-pyrrolo[3,2-b]pyridin-2-ol

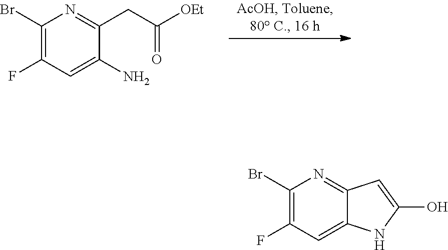 [SMILES: CCOC(=O)Cc1nc(Br)c(F)cc1N ; Oc1cc2nc(Br)c(F)cc2[nH]1]

To a stirred solution of ethyl 2-(3-amino-6-bromo-5-fluoropyridin-2-yl)acetate (3 g, 10.9 mmol, 1.0 eq) in Toluene (30 mL, 10 Vol) at room temperature, was added AcOH (3.1 mL, 54.4 mmol, 5 eq), and stirred at 80° C. for 16 h. After completion of reaction by TLC, reaction mixture was concentrated and quenched with aq. NaHCO₃ solution, precipitated solid was filtered, washed with pentane (20 mL) and dried under vacuum to afford 5-bromo-6-fluoro-1H-pyrrolo[3,2-b]pyridin-2-ol as a brown solid (2.2 g, Yield: 76%). TLC system: EtOAc/Hexane (50:50), R_{f} value: ^{˜}0.2; LCMS (m/z): 231.02 (M+H)⁺.

### Synthesis of 2,5-dibromo-6-fluoro-1H-pyrrolo[3,2-b]pyridine

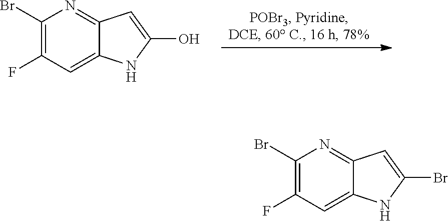 [SMILES: Oc1cc2nc(Br)c(F)cc2[nH]1 ; Fc1cc2[nH]c(Br)cc2nc1Br]

To a stirred solution of 5-bromo-6-fluoro-1H-pyrrolo[3,2-b]pyridin-2-ol (2.2 g, 9.56 mmol, 1.0 eq) in DCE (20 mL, 10 Vol) at 0° C., was added POBr₃ (5.5 g, 19.13 mmol, 2 eq), pyridine (1.1 mL, 14.34 mmol, 1.5 eq) and stirred at 60° C. for 16 h. After completion of reaction by TLC, reaction mixture was concentrated, quenched with aq. NaHCO₃ and extracted with EtOAc (2×100 mL). The combined organic layer was dried over Na₂SO₄, filtered and concentrated under reduced pressure. The crude product was triturated with n-pentane (30 mL) and dried to afford 2,5-dibromo-6-fluoro-1H-pyrrolo[3,2-b]pyridine as grey solid (2.2 g, Yield: 78%). TLC system: EtOAc/Hexane (20:80), R_{f} value: ^{˜}0.8; LCMS (m/z): 292.98 (M+H)⁺.

### Synthesis of 2,5-dibromo-6-fluoro-1-((2-(trimethylsilyl)ethoxy)methyl)-1H-pyrrolo[3,2-b]pyridine

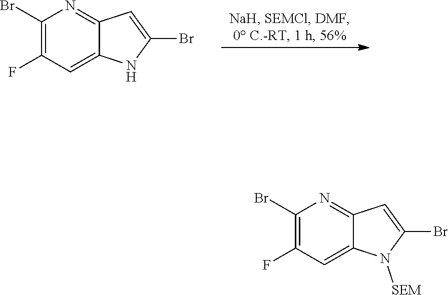 [SMILES: Fc1cc2[nH]c(Br)cc2nc1Br ; C[Si](C)(C)CCOCn1c(Br)cc2nc(Br)c(F)cc21]

To a stirred solution of 2,5-dibromo-6-fluoro-1H-pyrrolo[3,2-b]pyridine (2 g, 6.87 mmol, 1.0 eq) in DMF (20 mL, 10 Vol) at 0° C., was added 60% NaH (550 mg, 13.7 mmol, 2.0 eq) and stirred for 30 min. Later, added SEM-Cl (1.5 mL, 8.93 mmol, 1.3 eq) and allowed to room temperature, stirred for 1 h. After completion of reaction by TLC, the reaction mixture was diluted with ice cold water (80 mL) and extracted with EtOAc (2×100 mL). The combined organic layer was dried over Na₂SO₄, filtered and concentrated under reduced pressure. The crude product was purified by silica gel (60-120 mesh) column chromatography [elution with 10% EtOAc in Hexane] to afford 2,5-dibromo-6-fluoro-1-((2-(trimethylsilyl)ethoxy)methyl)-1H-pyrrolo[3,2-b]pyridine as a pale-yellow liquid (2 g, Yield: 56%). TLC system: EtOAc/Hexane (20:80), R_{f} value: ˜0.8; LCMS (m/z): 423.06 (M+H)⁺. ¹H NMR (400 MHz, CDCl₃) δ: 7.54 (d, J=8 Hz, 1H), 6.76 (s, 1H), 5.53 (s, 2H), 3.52 (t, J=8.2 Hz, 2H), 0.88 (t, J=8.2 Hz, 3H), −0.04 (s, 9H).

### Synthesis of 3-((5-bromo-6-fluoro-1-((2-(trimethylsilyl)ethoxy)methyl)-1H-pyrrolo[3,2-b]pyridin-2-yl)oxy)benzonitrile

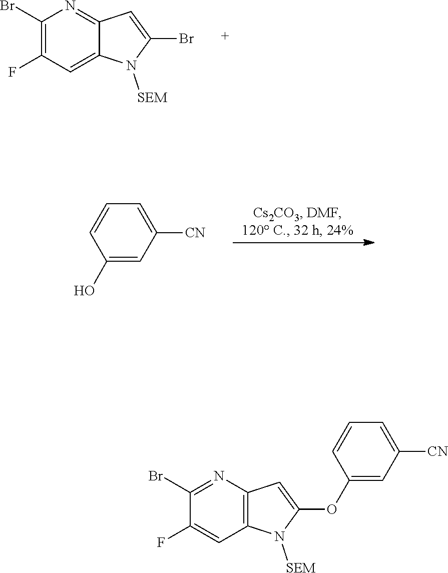 [SMILES: C[Si](C)(C)CCOCn1c(Br)cc2nc(Br)c(F)cc21 ; N#Cc1cccc(O)c1 ; C[Si](C)(C)CCOCn1c(Oc2cccc(C#N)c2)cc2nc(Br)c(F)cc21]

To a stirred solution of 2,5-dibromo-6-fluoro-1-((2-(trimethylsilyl)ethoxy)methyl)-1H-pyrrolo[3,2-b]pyridine (1.8 g, 4.26 mmol, 1.0 eq) and 3-hydroxybenzonitrile (660 mg, 5.54 mmol, 1.3 eq) in DMF (10 mL) at room temperature, was added Cs₂CO₃ (2.7 g, 8.52 mmol, 2.0 eq) and stirred at 120° C. for 32 h. After completion of reaction by TLC, the reaction mixture was diluted with water (50 mL) and extracted with EtOAc (2×100 mL). The combined organic layer was dried over Na₂SO₄, filtered and concentrated under reduced pressure. The crude product was purified by silica gel (100-200 mesh) column chromatography [elution with 10% EtOAc in Hexane]. The collected fractions were concentrated under reduced pressure to afford 3-((5-bromo-6-fluoro-1-((2-(trimethylsilyl)ethoxy)methyl)-1H-pyrrolo[3,2-b]pyridin-2-yl)oxy)benzonitrile as pale yellow liquid (480 mg, Yield: 24%). TLC system: EtOAc/Hexane (30:70), R_{f} value: ˜0.5; LCMS (m/z): 462.25 (M+H)⁺. ¹H NMR (400 MHz, CDCl₃) δ: 7.61-7.52 (m, 5H), 5.90 (s, 1H), 5.52 (s, 2H), 3.58 (t, J=8.2 H, 2H), 0.93 (t, J=8.2 H, 2H), −0.01 (s, 9H).

### Synthesis of (Z)-3-((5-bromo-6-fluoro-1-((2-(trimethylsilyl)ethoxy)methyl)-1H-pyrrolo[3,2-b]pyridin-2-yl)oxy)-N′-hydroxybenzimidamide

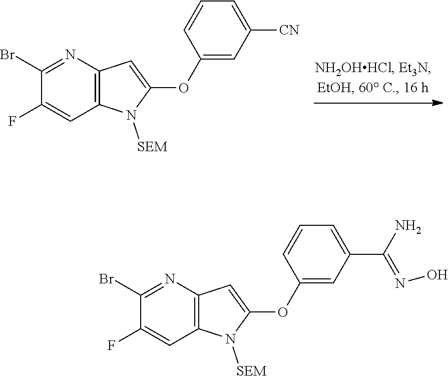 [SMILES: C[Si](C)(C)CCOCn1c(Oc2cccc(C#N)c2)cc2nc(Br)c(F)cc21 ; C[Si](C)(C)CCOCn1c(Oc2cccc(/C(N)=N/O)c2)cc2nc(Br)c(F)cc21]

To a stirred solution of 3-((5-bromo-6-fluoro-1-((2-(trimethylsilyl)ethoxy)methyl)-1H-pyrrolo[3,2-b]pyridin-2-yl)oxy)benzonitrile (630 mg, 1.37 mmol, 1.0 eq) in EtOH (22 mL) at room temperature, was added NH₂OH·HCl (190 mg, 2.73 mmol, 2 eq), Et₃N (0.6 mL, 4.10 mmol, 3.0 eq) and stirred at 60° C. for 3 h. After completion of reaction by TLC, reaction mixture was concentrated, diluted with ice cold water (20 mL) and extracted with EtOAc (2×50 mL). The combined organic layer was dried over Na₂SO₄, filtered and concentrated under reduced pressure to afford (Z)-3-((5-bromo-6-fluoro-1-((2-(trimethylsilyl)ethoxy)methyl)-1H-pyrrolo[3,2-b]pyridin-2-yl)oxy)-N′-hydroxybenzimidamide as colorless liquid (500 mg, crude). TLC system: EtOAc/Hexane (50:50), R_{f} value: ˜0.2; LCMS (m/z): 495.31 (M+H)⁺.

### Synthesis of ethyl (Z)-((3-((5-bromo-6-fluoro-1-((2-(trimethylsilyl)ethoxy)methyl)-1H-pyrrolo[3,2-b]pyridin-2-yl)oxy)phenyl)(hydroxyimino)methyl)carbamate

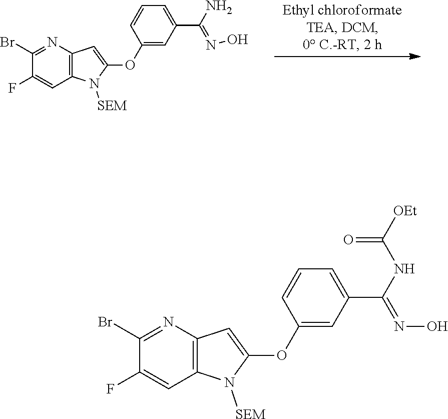 [SMILES: C[Si](C)(C)CCOCn1c(Oc2cccc(/C(N)=N/O)c2)cc2nc(Br)c(F)cc21 ; CCOC(=O)N/C(=N\O)c1cccc(Oc2cc3nc(Br)c(F)cc3n2COCC[Si](C)(C)C)c1]

To a stirred solution of (Z)-3-((5-bromo-6-fluoro-1-((2-(trimethylsilyl)ethoxy)methyl)-1H-pyrrolo[3,2-b]pyridin-2-yl)oxy)-N′-hydroxybenzimidamide (500 mg, 1.01 mmol, 1.0 eq) in DCM (10 mL) at 0° C. was added ethyl chloroformate (131 mg, 1.21 mmol, 1.2 eq), TEA (0.2 mL, 1.31 mmol, 1.3 eq) and stirred at room temperature for 2 h. After completion of reaction by TLC, reaction mixture concentrated under reduced pressure, diluted with ice cold water (20 mL) and extracted with EtOAc (2×50 mL), The combined organic layer was dried over Na₂SO₄, filtered and concentrated under reduced pressure to afford ethyl (Z)-((3-((5-bromo-6-fluoro-1-((2-(trimethylsilyl)ethoxy)methyl)-1H-pyrrolo[3,2-b]pyridin-2-yl)oxy)phenyl)(hydroxyimino)methyl)carbamate as off white solid (600 mg, crude). TLC system: EtOAc/Hexane (50:50), R_{f} value: ˜0.4; LCMS (m/z): 567.28 (M+H)⁺.

### Synthesis of 3-(3-((5-bromo-6-fluoro-1-((2-(trimethylsilyl)ethoxy)methyl)-1H-pyrrolo[3,2-b]pyridin-2-yl)oxy)phenyl)-1,2,4-oxadiazol-5(4H)-one

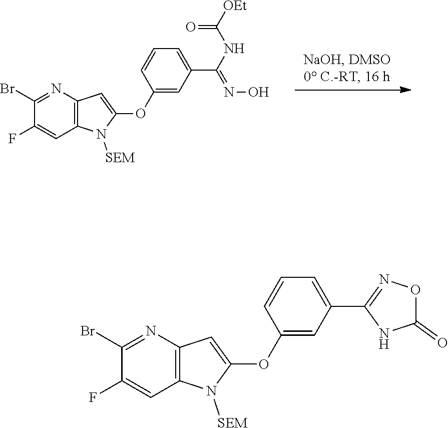

To a stirred solution of ethyl (Z)-((3-((5-bromo-6-fluoro-1-((2-(trimethylsilyl)ethoxy)methyl)-1H-pyrrolo[3,2-b]pyridin-2-yl)oxy)phenyl)(hydroxyimino)methyl)carbamate (600 mg, 1.06 mmol, 1.0 eq) in DMSO (3 mL, 5 Vol) at 0° C., was added NaOH (51 mg, 1.27 mmol, 1.3 eq), and stirred at RT for 16 h. After completion of reaction by TLC, the reaction mixture was acidified with 1N HCl (5 mL) and extracted with EtOAc (2×100 mL). The combined organic layer was dried over Na₂SO₄, filtered and concentrated under reduced pressure to afford 3-(3-((5-bromo-6-fluoro-1-((2-(trimethylsilyl)ethoxy)methyl)-1H-pyrrolo[3,2-b]pyridin-2-yl)oxy)phenyl)-1,2,4-oxadiazol-5(4H)-one as brown liquid (500 mg, Crude). TLC system: EtOAc/Hexane (50:50), R_{f} value: ˜0.1; LCMS (m/z): 521.27 (M+H)⁺.

### Synthesis of 3-(3-((5-(4-((dimethyl(oxo)-16-sulfaneylidene)amino)phenyl)-6-fluoro-1-((2-(trimethylsilyl)ethoxy)methyl)-1H-pyrrolo[3,2-b]pyridin-2-yl)oxy)phenyl)-1,2,4-oxadiazol-5(4H)-one

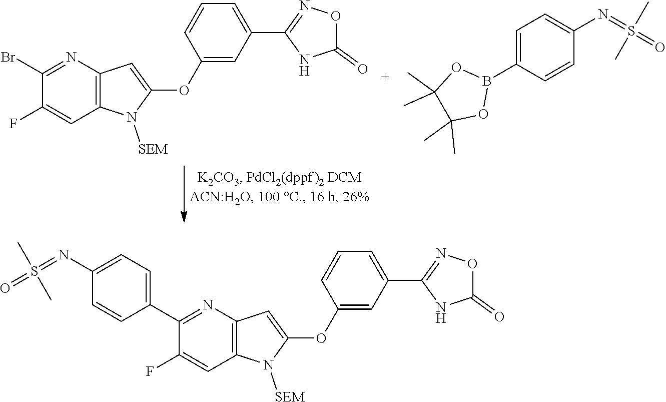

In a sealed tube, to a degassed solution of 3-(3-((5-bromo-6-fluoro-1-((2-(trimethylsilyl)ethoxy)methyl)-1H-pyrrolo[3,2-b]pyridin-2-yl)oxy)phenyl)-1,2,4-oxadiazol-5(4H)-one (500 mg, 0.96 mmol, 1.0 eq) and dimethyl ((4-(4,4,5,5-tetramethyl-1,3,2-dioxaborolan-2-yl)phenyl) imino)-16-sulfanone (570 mg, 1.92 mmol, 2 eq) in ACN:H₂O (1:1) (2 mL, 20 Vol) at room temperature, was added K₂CO₃ (398 mg, 2.88 mmol, 3.0 eq) followed by PdCl₂(dppf)·DCM (80 mg, 0.09 mmol, 0.1 eq) and stirred at 100° C. for 16 h. After completion of reaction by TLC, the reaction mixture was diluted with water (30 mL) and extracted with EtOAc (2×80 mL). The combined organic layer was dried over Na₂SO₄, filtered and concentrated under reduced pressure. Crude purified by reverse phase Column. [eluted with 60-65% ACN with 0.1% FA in H₂O] and fractions were evaporated under reduced pressure to afford 3-(3-((5-(4-((dimethyl(oxo)-16-sulfaneylidene)amino)phenyl)-6-fluoro-1-((2-(trimethylsilyl)ethoxy)methyl)-1H-pyrrolo[3,2-b]pyridin-2-yl)oxy)phenyl)-1,2,4-oxadiazol-5(4H)-one as brown gummy solid (220 mg, Yield: 26% in 4 steps). TLC system: EtOAc/Hexane (50:50), R_{f} value: ˜0.2; ¹H NMR (400 MHz, CDCl₃) δ: 7.67 (d, J=7.2 Hz, 2H), 7.61 (d, J=7.6 Hz, 1H), 7.55 (s, 1H), 7.52-7.45 (m, 2H), 7.36 (dd, J=8 Hz, 1.6 Hz, 1H), 7.10 (d, J=8.4 Hz, 2H), 5.89 (s, 1H), 5.42 (s, 2H), 3.55 (t, J=8.4 Hz, 2H), 3.18 (s, 6H), 0.89 (t, J=8.4 Hz, 3H), −0.05 (s, 9H).

### Synthesis of 3-(3-((5-(4-((dimethyl(oxo)-16-sulfaneylidene)amino)phenyl)-6-fluoro-1H-pyrrolo[3,2-b]pyridin-2-yl)oxy)phenyl)-1,2,4-oxadiazol-5(4H)-one

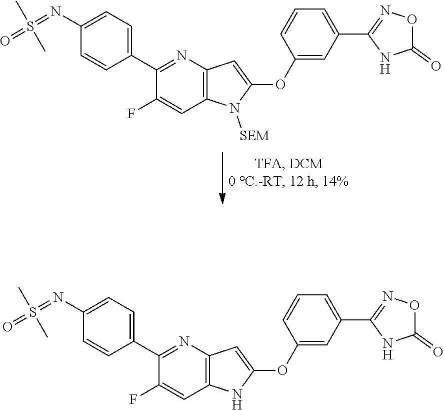

To a stirred solution of 3-(3-((5-(4-((dimethyl(oxo)-16-sulfaneylidene)amino)phenyl)-6-fluoro-1-((2-(trimethylsilyl)ethoxy)methyl)-1H-pyrrolo[3,2-b]pyridin-2-yl)oxy)phenyl)-1,2,4-oxadiazol-5(4H)-one (220 mg, 0.36 mmol, 1.0 eq) in DCM (4.5 mL, 20 Vol) at 0° C., was added TFA (4.4 mL, 10 Vol) and stirred at room temperature for 12 h. After completion of reaction by TLC, the reaction mixture was concentrated under reduced pressure and triturated with diethyl ether (10 mL), n-Pentane (10 mL) to provide crude product. Purified by Prep-HPLC and collected fractions were lyophilized to afford 3-(3-((5-(4-((dimethyl(oxo)-16-sulfaneylidene)amino)phenyl)-6-fluoro-1H-pyrrolo[3,2-b]pyridin-2-yl)oxy)phenyl)-1,2,4-oxadiazol-5(4H)-one as yellow solid (25 mg, Yield: 14%). TLC system: EtOAc/Hexane (100:00), R_{f} value: ˜0.3; LCMS (m/z): 480.30 (M+H)⁺; ¹H NMR (400 MHz, DMSO-d₆) δ: 13.0 (brs, 1H), 11.94 (s, 1H), 7.74-7.59 (m, 6H), 7.48-7.45 (m, 1H), 7.03-7.01 (m, 2H), 5.96 (s, 1H), 3.26 (s, 6H).

Prep-HPLC condition: YMC C-18 (250*19.0) mm 5mic, MP-(A): 10 Mm Ammonium bicarbonate in WATER MP-(B): ACN:WATER (80:20) Time: 0/25,15/60,25/98; diluent: Water/ACN/THF, Flow Rate: 12 ml/min.

### SYNTHETIC EXAMPLE 47

### Synthesis of Compound II-4

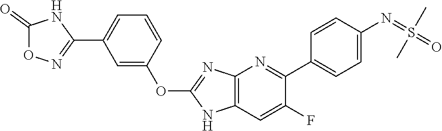 [SMILES: CS(C)(=O)=Nc1ccc(-c2nc3nc(Oc4cccc(-c5noc(=O)[nH]5)c4)[nH]c3cc2F)cc1]

### Synthesis of 5-fluoro-3-nitropyridin-2-amine

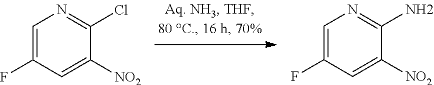 [SMILES: O=[N+]([O-])c1cc(F)cnc1Cl ; Nc1ncc(F)cc1[N+](=O)[O-]]

To a stirred solution of 2-chloro-5-fluoro-3-nitropyridine (8 g, 45.45 mol, 1.0 eq) in THF (40 mL, 5 Vol) at room temperature, was added Aq. NH₃ (48 mL, 6 Vol), and reaction mixture was allowed to stir at 80° C. for 16 h. After completion of reaction by TLC, the reaction mixture cooled to room temperature and evaporated under reduced pressure. The crude product was purified by neutral Al₂O₃ column chromatography [elution with 20% EtOAc in Hexane] to afford 5-fluoro-3-nitropyridin-2-amine as a yellow color solid (5 g, Yield: 70%). TLC system: EtOAc/Hexane (20:80), R_{f} value: ^{˜}0.3; LCMS (m/z): 158.06 (M+H)⁺; ¹H NMR (400 MHz, CDCl₃) δ: 8.32 (d, J=2.8 Hz, 1H), 8.20 (dd, J=5.2 Hz, 2.8 Hz, 1H), 6.67 (brs, 2H).

### Synthesis of 5-fluoropyridine-2,3-diamine

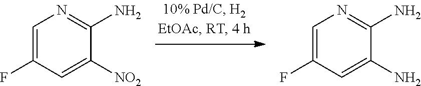 [SMILES: Nc1ncc(F)cc1[N+](=O)[O-] ; Nc1cc(F)cnc1N]

To a stirred of solution of 5-fluoro-3-nitropyridin-2-amine (5 g, 31.8 mmol, 1.0 eq) in EtOAc (100 mL, 20 Vol), was added 10% Pd/C (1 g) at room temperature and stirred for 4 h under H₂ balloon pressure. After completion of reaction by TLC, the reaction mixture was filtered through a pad of diatomaceous earth (e.g., Celite®), and washed with 10% MeOH/DCM (200 mL). Filtrate was concentrated under reduced pressure to afford 5-fluoropyridine-2,3-diamine as brown solid (4.2 g, crude). TLC system: EtOAc (100%), R_{f} value: ˜0.2; LCMS (m/z): 128.10 (M+H)⁺.

### Synthesis of 6-fluoro-1,3-dihydro-2H-imidazo[4,5-b]pyridin-2-one

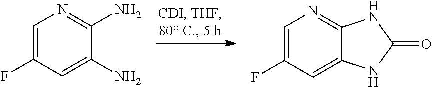 [SMILES: Nc1cc(F)cnc1N ; O=c1[nH]c2cc(F)cnc2[nH]1]

To a stirred of solution of 5-fluoropyridine-2,3-diamine (4.2 g, 33.1 mmol, 1.0 eq) in ACN (126 mL) at room temperature was added CDI (11 g, 66.2 mmol, 2.0 eq) and allowed to stir at 80° C. for 5 h. After completion of reaction by TLC, reaction mixture was acidified with 1N aq. HCl (100 mL), precipitated solid filtered, washed with diethyl ether (100 mL) and dried to afford 6-fluoro-1,3-dihydro-2H-imidazo[4,5-b]pyridin-2-one as ash color solid (3 g, yield: 62% in 2 steps). TLC system: EtOAc/Hexane (50:50), R_{f} value: ˜0.2; LCMS (m/z): 154.20 (M+H)⁺.

### Synthesis of 5-bromo-6-fluoro-1,3-dihydro-2H-imidazo[4,5-b]pyridin-2-one

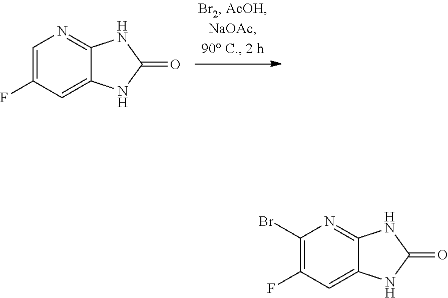 [SMILES: O=c1[nH]c2cc(F)cnc2[nH]1 ; O=c1[nH]c2cc(F)c(Br)nc2[nH]1]

To a stirred of solution of 6-fluoro-1,3-dihydro-2H-imidazo[4,5-b]pyridin-2-one (3 g, 19.6 mmol, 1.0 eq) in acetic acid (30 mL, 10 Vol) at room temperature was added NaOAc (3.2 g, 39.2 mmol, 2.0 eq), Br₂ (2 mL, 39.2 mmol, 2.0 eq) and reaction mixture was allowed to stir at 90° C. for 2 h. After completion of reaction by TLC, the reaction mixture was diluted with water (100 mL) and extracted with EtOAc (2×100 mL). The combined organic layer was washed with aq. NaHCO₃ solution, dried over Na₂SO₄, filtered and concentrated under reduced pressure to afford 5-bromo-6-fluoro-1,3-dihydro-2H-imidazo[4,5-b]pyridin-2-one as yellow solid (4.5 g, crude).TLC system: EtOAc/Hexane (50:50), R_{f} value: ˜0.3; LCMS (m/z): 232.03 (M+H)⁺.

### Synthesis of 5-bromo-2-chloro-6-fluoro-1H-imidazo[4,5-b]pyridine

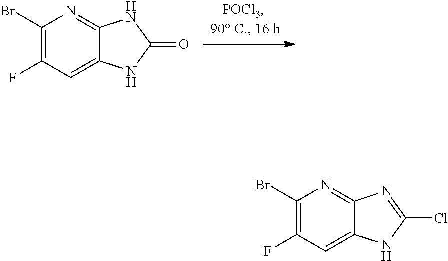 [SMILES: O=c1[nH]c2cc(F)c(Br)nc2[nH]1 ; Fc1cc2[nH]c(Cl)nc2nc1Br]

A solution of 5-bromo-6-fluoro-1,3-dihydro-2H-imidazo[4,5-b]pyridin-2-one (3 g, 12.98 mmol, 1.0 eq) in POCl₃ (30 mL, 10 Vol) was heated at 90° C. for 16 h. After completion of reaction by TLC, the reaction mixture cooled to room temperature, evaporated reduced pressure, diluted with water and extracted with EtOAc (2×250 mL). The combined organic layer was washed with sat. NaHCO₃ solution, dried over Na₂SO₄, filtered and concentrated under reduced pressure to afford 5-bromo-2-chloro-6-fluoro-1H-imidazo[4,5-b]pyridine as brown color solid (2 g, crude). TLC system: EtOAc/Hexane (50:50), R_{f} value: ˜0.4; LCMS (m/z): 247.95 (M−H)⁺.

### Synthesis of 5-bromo-2-chloro-6-fluoro-1-((2-(trimethylsilyl)ethoxy)methyl)-1H-imidazo[4,5-b]pyridine

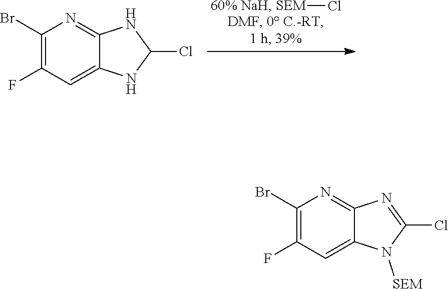

To a stirred solution of 5-bromo-2-chloro-6-fluoro-1H-imidazo[4,5-b]pyridine (1.7 g, 6.8 mmol, 1.0 eq) in DMF (34 mL, 20 Vol) at 0° C., was added 60% NaH (326 mg, 13.6 mmol, 2.0 eq) and stirred for 30 min. After 30 min was added SEM-Cl (1.8 mL, 10.2 mmol, 1.5 eq) and reaction mixture was allowed to stir at room temperature for 1 h. After completion of reaction by TLC, the reaction mixture was diluted with ice cold water (90 mL) and extracted with EtOAc (2×300 mL). The combined organic layer was dried over Na₂SO₄, filtered and concentrated under reduced pressure to afford crude product. The crude product was purified by silica gel (100-200 mesh) column chromatography [elution with 20% EtOAc in Hexane] to afford 5-bromo-2-chloro-6-fluoro-1-((2-(trimethylsilyl)ethoxy)methyl)-1H-imidazo[4,5-b]pyridine as a colorless solid (1 g, Yield: 39%). TLC system: EtOAc/Hexane (20:80), R_{f} value: ˜0.3; LCMS (m/z): 380.07 (M+H)⁺; observed not clean. ¹H NMR (400 MHz, CDCl₃) δ: 7.74 (d, J=7.6 Hz, 1H), 5.64 (s, 1H), 3.68-3.64 (m, 2H), 0.99-0.86 (m, 2H), −0.01 (s, 9H).

### Synthesis of 3-(3-((5-bromo-6-fluoro-1-((2-(trimethylsilyl)ethoxy)methyl)-1H-imidazo[4,5-b]pyridin-2-yl)oxy)phenyl)-1,2,4-oxadiazol-5(4H)-one

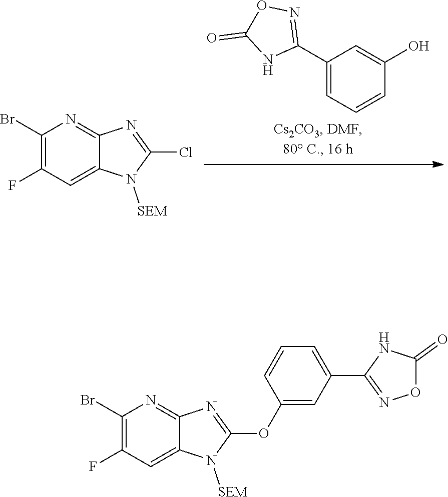 [SMILES: O=c1[nH]c(-c2cccc(O)c2)no1 ; C[Si](C)(C)CCOCn1c(Cl)nc2nc(Br)c(F)cc21 ; C[Si](C)(C)CCOCn1c(Oc2cccc(-c3noc(=O)[nH]3)c2)nc2nc(Br)c(F)cc21]

To a stirred solution of 5-bromo-2-chloro-6-fluoro-1-((2-(trimethylsilyl)ethoxy)methyl)-1H-imidazo[4,5-b]pyridine (150 mg, 0.39 mmol, 1.0 eq) and 3-(3-hydroxyphenyl)-1,2,4-oxadiazol-5(4H)-one (91.5 mg, 0.51 mmol, 1.3 eq) in DMF (1.5 mL, 10 Vol) at room temperature, was added Cs₂CO₃ (258 mg, 0.79 mmol, 2.0 eq) and stirred at 80° C. for 16 h. After completion of reaction by TLC, the reaction mixture was diluted with water (30 mL) and extracted with EtOAc (2×90 mL). The combined organic layer was dried over Na₂SO₄, filtered and concentrated under reduced pressure to afford 3-(3-((5-bromo-6-fluoro-1-((2-(trimethylsilyl)ethoxy)methyl)-1H-imidazo[4,5-b]pyridin-2-yl)oxy)phenyl)-1,2,4-oxadiazol-5(4H)-one as a colorless gummy liquid (220 mg, crude).TLC system: EtOAc/Hexane (50:50), R_{f} value: ˜0.2; LCMS (m/z): 522.23 (M+H)⁺; ionization was low and observed desired mass as small peaks.

### Synthesis of 3-(3-((5-(4-((dimethyl(oxo)-16-sulfaneylidene)amino)phenyl)-6-fluoro-1-((2-(trimethylsilyl)ethoxy)methyl)-1H-imidazo[4,5-b]pyridin-2-yl)oxy)phenyl)-1,2,4-oxadiazol-5(4H)-one

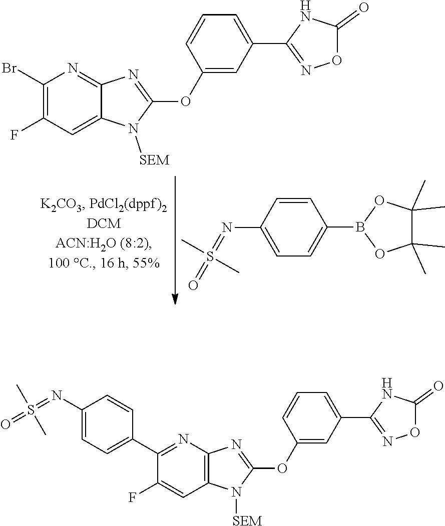

In a sealed tube, to a degassed solution of 3-(3-((5-bromo-6-fluoro-1-((2-(trimethylsilyl)ethoxy)methyl)-1H-imidazo[4,5-b]pyridin-2-yl)oxy)phenyl)-1,2,4-oxadiazol-5(4H)-one (220 mg, 0.42 mmol, 1.0 eq) and dimethyl ((4-(4,4,5,5-tetramethyl-1,3,2-dioxaborolan-2-yl)phenyl) imino)-16-sulfanone (311.5 mg, 1.05 mmol, 2.5 eq) in ACN:H₂O (8:2) (4.4 mL, 20 Vol) at room temperature, was added K₂CO₃ (174.8 mg, 1.26 mmol, 3.0 eq) followed by PdCl₂(dppf)·DCM (34 mg, 0.042 mmol, 0.1 eq) and stirred at 100° C. for 16 h. After completion of reaction by TLC, the reaction mixture was diluted with water (30 mL) and extracted with EtOAc (2×100 mL). The combined organic layer was dried over Na₂SO₄, filtered and concentrated under reduced pressure. Crude was purified by revere phase column and collected fractions were evaporated under reduced pressure to 3-(3-((5-(4-((dimethyl(oxo)-16-sulfaneylidene)amino)phenyl)-6-fluoro-1-((2-(trimethylsilyl)ethoxy)methyl)-1H-imidazo[4,5-b]pyridin-2-yl)oxy)phenyl)-1,2,4-oxadiazol-5(4H)-one as a off white solid (130 mg, Yield: 55% in 2 steps). TLC system: EtOAc (100%), R_{f} value: ˜0.2; LCMS (m/z): 611.40 (M+H)⁺.

### Synthesis of 3-(3-((5-(4-((dimethyl(oxo)-16-sulfaneylidene)amino)phenyl)-6-fluoro-1H-imidazo[4,5-b]pyridin-2-yl)oxy)phenyl)-1,2,4-oxadiazol-5(4H)-one

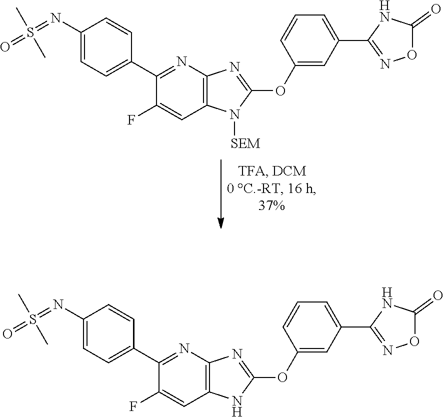 [SMILES: C[Si](C)(C)CCOCn1c(Oc2cccc(-c3noc(=O)[nH]3)c2)nc2nc(-c3ccc(N=S(C)(C)=O)cc3)c(F)cc21 ; CS(C)(=O)=Nc1ccc(-c2nc3nc(Oc4cccc(-c5noc(=O)[nH]5)c4)[nH]c3cc2F)cc1]

To a stirred solution 3-(3-((5-(4-((dimethyl(oxo)-16-sulfaneylidene)amino)phenyl)-6-fluoro-1-((2-(trimethylsilyl)ethoxy)methyl)-1H-imidazo[4,5-b]pyridin-2-yl)oxy)phenyl)-1,2,4-oxadiazol-5(4H)-one (130 mg, 0.21 mmol, 1.0 eq) in DCM (2.6 mL, 20 Vol) at 0° C., was added TFA (1.3 mL, 10 Vol) and stirred at room temperature. After completion of reaction by TLC, the reaction mixture concentrated under reduced pressure and purified by prep-HPLC to afford 3-(3-((5-(4-((dimethyl(oxo)-16-sulfaneylidene)amino)phenyl)-6-fluoro-1H-imidazo[4,5-b]pyridin-2-yl)oxy)phenyl)-1,2,4-oxadiazol-5(4H)-one as off white solid (37.2 mg, Yield: 37%). TLC system: EtOAc (100%), R_{f} value: ˜0.2; LCMS (m/z): 479.20 (M+H)⁺; ¹H NMR (400 MHz, DMSO-d₆) δ: 7.80-7.71 (m, 5H), 7.52 (t, J=8.0 Hz, 1H), 7.47-7.44 (m, 1H), 7.05-7.02 (m, 2H), 3.27 (s, 6H).

Prep-Conditions: Column: GEMINI NX C18 (250*21.2) mm 5mic; Buffer MP-(A): 0.1% FA in WATER, MP-(B): ACN:WATER (80:20); Gradient (T/% B): 0/10, 15/40, 20/95, 25/98; Flow Rate: 13 ml/min; Solubility: ACN, water, THF

### SYNTHETIC EXAMPLE 48

### Synthesis of Compound II-6

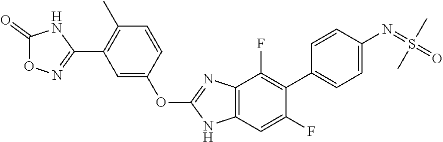 [SMILES: Cc1ccc(Oc2nc3c(F)c(-c4ccc(N=S(C)(C)=O)cc4)c(F)cc3[nH]2)cc1-c1noc(=O)[nH]1]

### Synthesis of (Z)-N′,3-dihydroxy-4-methylbenzimidamide

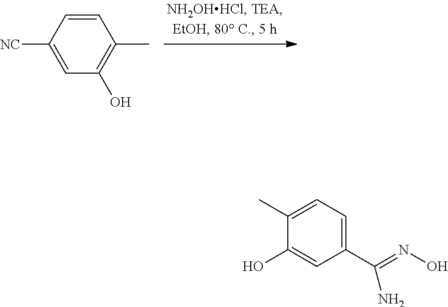

To a stirred solution of 3-hydroxy-4-methylbenzonitrile (1.0 g, 7.5 mmol, 1.0 eq) in EtOH (35 mL, 35 Vol) at room temperature, was added TEA (2.5 mL, 22.5 mmol, 3.0 eq), NH₂OH·HCl (1.0 g, 15.0 mmol, 2.0 eq) and stirred at 80° C. for 5 h. After completion of reaction by TLC, the reaction mixture was concentrated under reduced pressure to afford (Z)-N′,3-dihydroxy-4-methylbenzimidamide as a brown gummy solid (1.1 g, crude). TLC system: EtOAc (100%), R_{f} value: ˜0.1; LCMS (m/z): 167.23 (M+H)⁺.

### Synthesis of 3-(3-hydroxy-4-methylphenyl)-1,2,4-oxadiazol-5(4H)-one

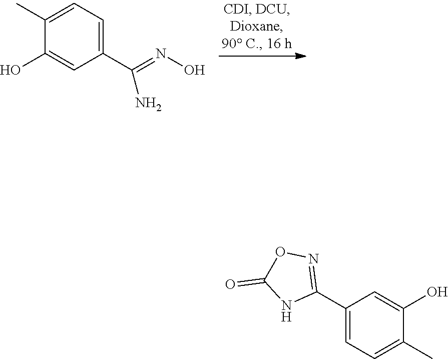

To a stirred solution of (Z)-N′,3-dihydroxy-4-methylbenzimidamide (1.1 g, 6.6 mmol, 1.0 eq) in dioxane (11 mL, 10 Vol) at room temperature, was added DBU (1.0 mL, 7.3 mmol, 1.1 eq), CDI (1.2 g, 7.3 mmol, 1.1 eq) and stirred at 90° C. for 16 h. After completion of reaction by TLC, the reaction mixture was evaporated under reduced pressure, acidified with 2N aq. HCl and extracted with EtOAc (2×100 mL). The combined organic layer was dried over Na₂SO₄ and concentrated under reduced pressure to afford 3-(3-hydroxy-4-methylphenyl)-1,2,4-oxadiazol-5(4H)-one as colorless solid (1.1 g, crude). TLC system: EtOAc/Hexane (50:50%), R_{f} value: ˜0.2; LCMS (m/z): 193.21 (M+H)⁺.

### Synthesis of 3-(3-((6-bromo-5,7-difluoro-1-((2-(trimethylsilyl)ethoxy)methyl)-1H-benzo[d]imidazol-2-yl)oxy)-4-methylphenyl)-1,2,4-oxadiazol-5(4H)-one

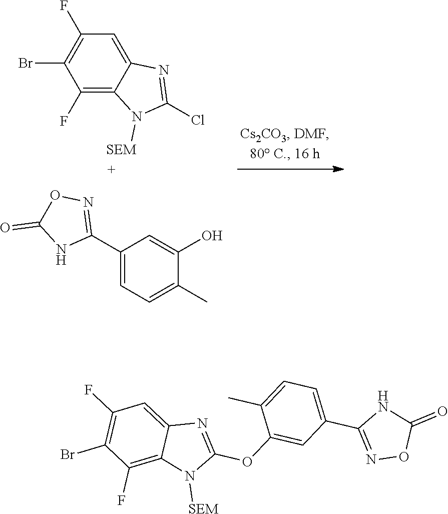 [SMILES: C[Si](C)(C)CCOCn1c(Cl)nc2cc(F)c(Br)c(F)c21 ; Cc1ccc(-c2noc(=O)[nH]2)cc1O ; Cc1ccc(-c2noc(=O)[nH]2)cc1Oc1nc2cc(F)c(Br)c(F)c2n1COCC[Si](C)(C)C]

To a stirred solution of 6-bromo-2-chloro-5,7-difluoro-1-((2-(trimethylsilyl)ethoxy)methyl)-1H-benzo[d]imidazole (1.2 g, 3.0 mmol, 1.0 eq) and 3-(3-hydroxy-4-methylphenyl)-1,2,4-oxadiazol-5(4H)-one (699.4 mg, 3.92 mmol, 1.3 eq) in DMF (12 mL, 10 Vol) at room temperature, was added Cs₂CO₃ (1.97 g, 6.04 mmol, 2.0 eq) and stirred at 80° C. for 16 h. After completion of reaction by TLC, the reaction mixture was diluted with water (100 mL) and extracted with EtOAc (2×150 mL). The combined organic layer was dried over Na₂SO₄, filtered and concentrated under reduced pressure to afford 3-(3-((6-bromo-5,7-difluoro-1-((2-(trimethylsilyl)ethoxy)methyl)-1H-benzo[d]imidazol-2-yl)oxy)-4-methylphenyl)-1,2,4-oxadiazol-5(4H)-one as a colorless solid (1.1 g, crude).TLC system: EtOAc/Hexane (50:50), R_{f} value: ˜0.2; LCMS (m/z): 553.32 (M+H)⁺.

### Synthesis of 3-(3-((5-(4-((dimethyl(oxo)-16-sulfaneylidene)amino)phenyl)-4,6-difluoro-1-((2-(trimethylsilyl)ethoxy)methyl)-1H-benzo[d]imidazol-2-yl)oxy)-4-methylphenyl)-1,2,4-oxadiazol-5(4H)-one

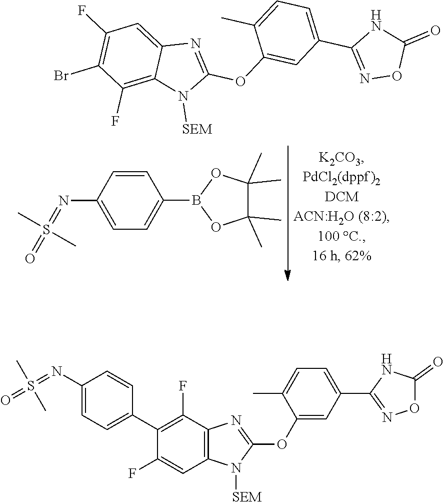

To a stirred solution of 3-(3-((6-bromo-5,7-difluoro-1-((2-(trimethylsilyl)ethoxy)methyl)-1H-benzo[d]imidazol-2-yl)oxy)-4-methylphenyl)-1,2,4-oxadiazol-5(4H)-one (300 mg, 0.57 mmol, 1.0 eq) and dimethyl ((4-(4,4,5,5-tetramethyl-1,3,2-dioxaborolan-2-yl)phenyl) imino)-16-sulfanone (423.8 mg, 1.43 mmol, 2.5 eq) in ACN:H₂O (8:2) (6 mL, 20 Vol) at room temperature, was added K₂CO₃ (238 mg, 1.72 mmol, 3.0 eq) followed by PdCl₂(dppf)·DCM (47 mg, 0.057 mmol, 0.1 eq) and stirred at 100° C. for 16 h. After completion of reaction by TLC, the reaction mixture was diluted with water (20 mL) and extracted with EtOAc (2×70 mL). The combined organic layer was dried over Na₂SO₄, filtered and concentrated under reduced pressure to afford crude product. Obtained material was purified by silica column chromatography to afford 3-(3-((5-(4-((dimethyl(oxo)-16-sulfaneylidene)amino)phenyl)-4,6-difluoro-1-((2-(trimethylsilyl)ethoxy)methyl)-1H-benzo[d]imidazol-2-yl)oxy)-4-methylphenyl)-1,2,4-oxadiazol-5(4H)-one as colorless solid (230 mg, Yield: 62%). TLC system: EtOAc (100), R_{f} value: ˜0.1; LCMS (m/z): 642.44 (M+H)⁺.

### Synthesis of 3-(3-((5-(4-((dimethyl(oxo)-16-sulfaneylidene)amino)phenyl)-4,6-difluoro-1H-benzo[d]imidazol-2-yl)oxy)-4-methylphenyl)-1,2,4-oxadiazol-5(4H)-one

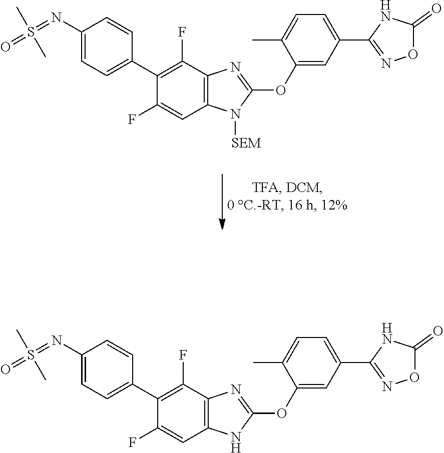 [SMILES: Cc1ccc(-c2noc(=O)[nH]2)cc1Oc1nc2c(F)c(-c3ccc(N=S(C)(C)=O)cc3)c(F)cc2n1COCC[Si](C)(C)C ; Cc1ccc(-c2noc(=O)[nH]2)cc1Oc1nc2c(F)c(-c3ccc(N=S(C)(C)=O)cc3)c(F)cc2[nH]1]

To a stirred solution of 3-(3-((5-(4-((dimethyl(oxo)-16-sulfaneylidene)amino)phenyl)-4,6-difluoro-1-((2-(trimethylsilyl)ethoxy)methyl)-1H-benzo[d]imidazol-2-yl)oxy)-4-methylphenyl)-1,2,4-oxadiazol-5(4H)-one (230 mg, 0.35 mmol, 1.0 eq) in DCM (4.6 mL, 20 Vol) at 0° C., was added TFA (2.3 mL, 10 Vol) and stirred at room temperature. After completion of reaction by TLC, the reaction mixture was concentrated, washed with pentane (20 mL) and purified by Prep-HPLC. The collected fractions were lyophilized to afford 3-(3-((5-(4-((dimethyl(oxo)-16-sulfaneylidene)amino)phenyl)-4,6-difluoro-1H-benzo[d]imidazol-2-yl)oxy)-4-methylphenyl)-1,2,4-oxadiazol-5(4H)-one as an off white solid (21.3 mg, Yield: 12%). TLC system: 10% MeOH/DCM (10:90), R_{f} value: ˜0.1; LCMS (m/z): 512.30 (M+H)⁺; ¹H NMR (400 MHz, DMSO-d₆) δ: 13.07 (brs, 1H), 7.76-7.75 (m, 1H), 7.70-7.67 (m, 1H), 7.59-7.56 (m, 1H), 7.26-7.19 (m, 3H), 7.02 (d, J=8.4 Hz, 2H), 3.26 (s, 6H), 2.29 (s, 3H).

Prep HPLC Method: YMC C-18 (250*20.0) mm 5mic, A: MP-(A): 10 mM ABC IN Water MP-(B): ACN:Water (80:20), Gradient (T/% B): 0/25, 15/60, 20/90, 25/98; Solubility: Water/ACN/THF, Flow Rate: 13 ml/min.

### SYNTHETIC EXAMPLE 49

### Synthesis of Compound II-8

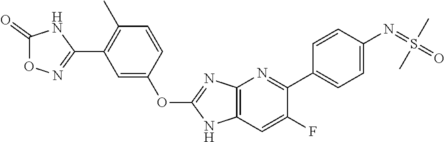 [SMILES: Cc1ccc(Oc2nc3nc(-c4ccc(N=S(C)(C)=O)cc4)c(F)cc3[nH]2)cc1-c1noc(=O)[nH]1]

### Synthesis of 3-(5-((5-(4-((dimethyl(oxo)-16-sulfaneylidene)amino)phenyl)-6-fluoro-1-((2-(trimethylsilyl)ethoxy)methyl)-1H-imidazo[4,5-b]pyridin-2-yl)oxy)-2-methylphenyl)-1,2,4-oxadiazol-5(4H)-one

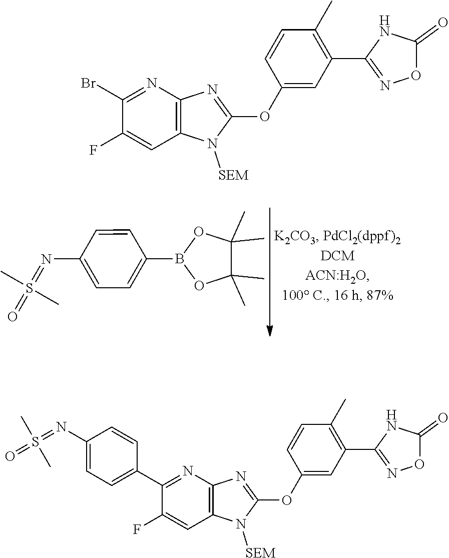

In a sealed tube to a degassed solution of 3-(5-((5-bromo-6-fluoro-1-((2-(trimethylsilyl)ethoxy)methyl)-1H-imidazo[4,5-b]pyridin-2-yl)oxy)-2-methylphenyl)-1,2,4-oxadiazol-5(4H)-one (200 mg, 0.37 mmol, 1.0 eq) and dimethyl ((4-(4,4,5,5-tetramethyl-1,3,2-dioxaborolan-2-yl)phenyl) imino)-16-sulfanone (276 mg, 0.93 mmol, 2.5 eq) in ACN:H₂O (4:1, 20 Vol) at room temperature, was added K₂CO₃ (153 mg, 1.11 mmol, 3.0 eq) followed by Pd(dppf)Cl₂·DCM (30 mg, 0.037 mmol, 0.1 eq) and stirred at 100° C. for 16 h. After completion of reaction by TLC, the reaction mixture was diluted with water (10 mL) and extracted with EtOAc (2×10 mL). The combined organic layer was dried over Na₂SO₄, filtered and concentrated under reduced pressure to afford crude product. The crude product was purified by silica gel [100-200] column chromatography [elution with 3% MeOH in DCM] to afford 3-(5-((5-(4-((dimethyl(oxo)-16-sulfaneylidene)amino)phenyl)-6-fluoro-1-((2-(trimethylsilyl)ethoxy)methyl)-1H-imidazo[4,5-b]pyridin-2-yl)oxy)-2-methylphenyl)-1,2,4-oxadiazol-5(4H)-one as pale brown gum (200 mg, Yield: 87%). TLC system: EtOAc/Hexane (100:00), R_{f} value: ˜0.5; LCMS (m/z): 625.47 (M+H)⁺; ¹H NMR (400 MHz, CDCl₃) δ: 8.39 (s, 1H), 7.97 (d, J=8.4 Hz, 2H), 7.48-7.36 (m, 2H), 7.23-7.11 (m, 2H), 7.08-7.05 (m, 1H), 4.69 (s, 2H), 3.45-3.37 (m, 2H), 3.21 (s, 6H), 2.62 (s, 3H), 0.83-0.79 (m, 2H), 0.03 (s, 9H). NMR was having excess aliphatic protons.

### Synthesis of 3-(5-((5-(4-((dimethyl(oxo)-16-sulfaneylidene)amino)phenyl)-6-fluoro-1H-imidazo[4,5-b]pyridin-2-yl)oxy)-2-methylphenyl)-1,2,4-oxadiazol-5(4H)-one

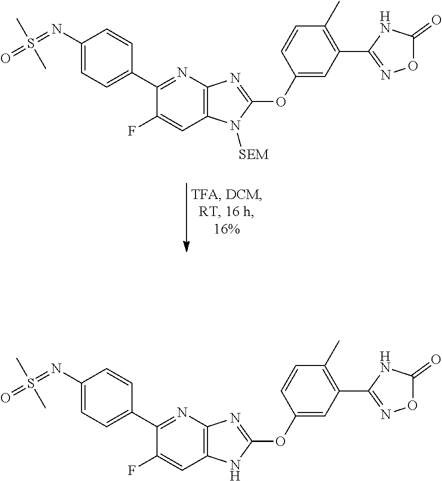 [SMILES: Cc1ccc(Oc2nc3nc(-c4ccc(N=S(C)(C)=O)cc4)c(F)cc3n2COCC[Si](C)(C)C)cc1-c1noc(=O)[nH]1 ; Cc1ccc(Oc2nc3nc(-c4ccc(N=S(C)(C)=O)cc4)c(F)cc3[nH]2)cc1-c1noc(=O)[nH]1]

To a stirred solution of 3-(5-((5-(4-((dimethyl(oxo)-16-sulfaneylidene)amino)phenyl)-6-fluoro-1-((2-(trimethylsilyl)ethoxy)methyl)-1H-imidazo[4,5-b]pyridin-2-yl)oxy)-2-methylphenyl)-1,2,4-oxadiazol-5(4H)-one (200 mg, 0.32 mmol, 1.0 eq) in DCM (10 Vol) at 0° C., was added TFA (1.0 mL) and stirred at room temperature for 16 h. After completion of reaction by TLC, the reaction mixture was concentrated under reduced pressure and purified by reverse phase column chromatography [elution with 90% ACN in 0.1% FA in H₂O], collected fractions were lyophilized to afford desired product. Material was further triturated with n-pentane (30 mL), decanted and dried under reduced pressure to afford 3-(5-((5-(4-((dimethyl(oxo)-16-sulfaneylidene)amino)phenyl)-6-fluoro-1H-imidazo[4,5-b]pyridin-2-yl)oxy)-2-methylphenyl)-1,2,4-oxadiazol-5(4H)-one as light brown solid (26 mg, Yield: 16%). TLC system: MeOH/DCM (10:90), R_{f} value: ˜0.2; LCMS (m/z): 495.41 (M+H)⁺; ¹H NMR (400 MHz, DMSO-d₆) δ: 12.79 (brs, 1H), 7.80 (d, J=12.0 Hz, 1H), 7.75 (dd, J=1.6 Hz, 8.8 Hz, 2H), 7.68-7.67 (m, 1H), 7.63 (dd, J=2.4 Hz, 8.4 Hz, 1H), 7.54 (d, J=8.8 Hz, 1H), 7.06-7.04 (m, 2H), 3.28 (s, 6H), 2.51 (s, 3H).

### SYNTHETIC EXAMPLE 50

### Synthesis of Compound II-10

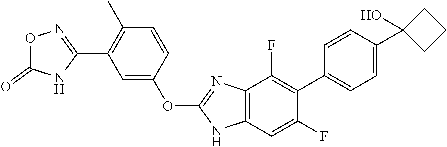 [SMILES: Cc1ccc(Oc2nc3c(F)c(-c4ccc(C5(O)CCC5)cc4)c(F)cc3[nH]2)cc1-c1noc(=O)[nH]1]

### Synthesis of 3-(3-((4,6-difluoro-5-(4-(1-hydroxycyclobutyl)phenyl)-1-((2-(trimethylsilyl)ethoxy)methyl)-1H-benzo[d]imidazol-2-yl)oxy)-4-methylphenyl)-1,2,4-oxadiazol-5(4H)-one

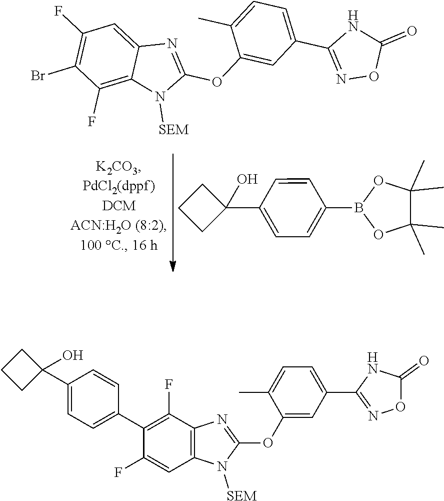 [SMILES: Cc1ccc(-c2noc(=O)[nH]2)cc1Oc1nc2cc(F)c(Br)c(F)c2n1COCC[Si](C)(C)C ; CC1(C)OB(c2ccc(C3(O)CCC3)cc2)OC1(C)C ; Cc1ccc(-c2noc(=O)[nH]2)cc1Oc1nc2c(F)c(-c3ccc(C4(O)CCC4)cc3)c(F)cc2n1COCC[Si](C)(C)C]

In a sealed tube to a degassed solution of 3-(3-((6-bromo-5,7-difluoro-1-((2-(trimethylsilyl)ethoxy)methyl)-1H-benzo[d]imidazol-2-yl)oxy)-4-methylphenyl)-1,2,4-oxadiazol-5(4H)-one (300 mg, 0.54 mmol, 1.0 eq) and 1-(4-(4,4,5,5-tetramethyl-1,3,2-dioxaborolan-2-yl)phenyl)cyclobutan-1-ol (372.5 mg, 1.36 mmol, 2.5 eq) in ACN:H₂O (8:2) (6 mL, 20 vol) at room temperature, was added K₂CO₃ (225 mg, 1.63 mmol, 3.0 eq) followed by PdCl₂(dppf)·DCM (44 mg, 0.054 mmol, 0.1 eq) and stirred at 100° C. for 16 h. After completion of reaction by TLC, the reaction mixture was diluted with water (20 mL) and extracted with EtOAc (2×100 mL). The combined organic layer was dried over Na₂SO₄, filtered and concentrated under reduced. Obtained crude was purified by reverse phase column chromatography to afford 3-(3-((4,6-difluoro-5-(4-(1-hydroxycyclobutyl)phenyl)-1-((2-(trimethylsilyl)ethoxy)methyl)-1H-benzo[d]imidazol-2-yl)oxy)-4-methylphenyl)-1,2,4-oxadiazol-5(4H)-one as a colorless solid (170 mg, Yield: 51%). TLC system: EtOAc/Hexane (50:50), R_{f} value: ˜0.2; LCMS (m/z): 621.33 (M+H)⁺.

### Synthesis of 3-(3-((4,6-difluoro-5-(4-(1-hydroxycyclobutyl)phenyl)-1H-benzo[d]imidazol-2-yl)oxy)-4-methylphenyl)-1,2,4-oxadiazol-5(4H)-one

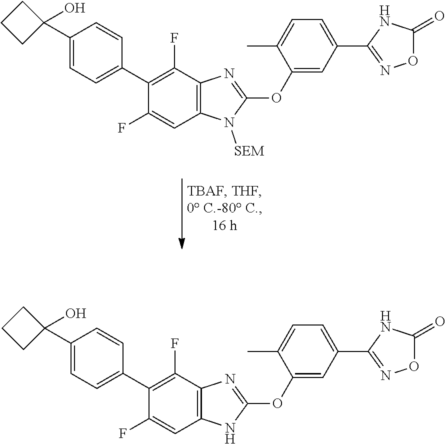 [SMILES: Cc1ccc(-c2noc(=O)[nH]2)cc1Oc1nc2c(F)c(-c3ccc(C4(O)CCC4)cc3)c(F)cc2n1COCC[Si](C)(C)C ; Cc1ccc(-c2noc(=O)[nH]2)cc1Oc1nc2c(F)c(-c3ccc(C4(O)CCC4)cc3)c(F)cc2[nH]1]

To a stirred solution of 3-(3-((4,6-difluoro-5-(4-(1-hydroxycyclobutyl)phenyl)-1-((2-(trimethylsilyl)ethoxy)methyl)-1H-benzo[d]imidazol-2-yl)oxy)-4-methylphenyl)-1,2,4-oxadiazol-5(4H)-one (150 mg, 0.24 mmol, 1.0 eq) in THF (1.5 mL, 10 vol) at 0° C., was added TBAF (1.5 mL, 10 vol) and stirred at 80° C. for 16 h. After completion of reaction by TLC, the reaction mixture concentrated under reduced pressure and washed with pentane (20 mL) to afford crude compound. Purified by Prep-HPLC and collected fractions were lyophilized to afford 3-(3-((4,6-difluoro-5-(4-(1-hydroxycyclobutyl)phenyl)-1H-benzo[d]imidazol-2-yl)oxy)-4-methylphenyl)-1,2,4-oxadiazol-5(4H)-one as off white solid (68 mg, Yield: 54%). TLC system: EtOAc/Hexane (50:50), R_{f} value: ˜0.1; LCMS (m/z): 491.30 (M+H)⁺; ¹H NMR (400 MHz, DMSO-d₆) δ: 13.95-12.20 (brs, 1H), 7.73 (s, 1H), 7.69-7.66 (m, 1H), 7.58 (d, J=8.4 Hz, 2H), 7.54 (d, J=8.0 Hz, 1H), 7.40 (d, J=8.0 Hz, 2H), 7.23 (d, J=9.6 Hz, 1H), 7.08 (brs, 1H), 5.55 (s, 1H), 2.50-2.41 (m, 2H), 2.33-2.26 (m, 5H), 1.98-1.89 (m, 1H), 1.73-1.70 (m, 1H).

Prep-HPLC condition: YMC TRIART C-18 (250*20.0) mm 5mic, A: MP-(A): 10 mM ABC IN Water MP-(B): ACN:Water (80:20), Gradient (T/% B): 0/40, 14/58, 15/58, 20/90, 25/98; Solubility: Water/ACN, Flow Rate: 13 ml/min.

### SYNTHETIC EXAMPLE 51

### Synthesis of Compound II-11

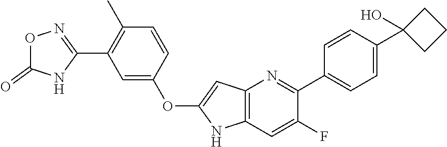 [SMILES: Cc1ccc(Oc2cc3nc(-c4ccc(C5(O)CCC5)cc4)c(F)cc3[nH]2)cc1-c1noc(=O)[nH]1]

### Synthesis of 5-((5-bromo-6-fluoro-1-((2-(trimethylsilyl)ethoxy)methyl)-1H-pyrrolo[3,2-b]pyridin-2-yl)oxy)-2-methylbenzonitrile

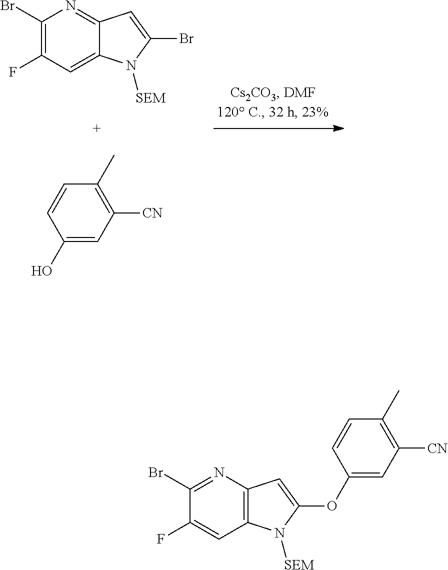

To a stirred solution of 2,5-dibromo-6-fluoro-1-((2-(trimethylsilyl)ethoxy)methyl)-1H-pyrrolo[3,2-b]pyridine (2 g, 4.74 mmol, 1.0 eq) in DMF (40 mL, 20 Vol) at room temperature, was added Cs₂CO₃ (3.1 g, 9.47 mmol, 2.0 eq) followed by 5-hydroxy-2-methylbenzonitrile (820 mg, 6.16 mmol, 1.3 eq)) and stirred at 120° C. for 32 h. After completion of reaction by TLC, the reaction mixture was diluted with water (100 mL) and extracted with EtOAc (2×150 mL). The combined organic layer was dried over Na₂SO₄, filtered and concentrated under reduced pressure afford crude product. The crude product was purified by silica gel (100-200 mesh) column chromatography [elution with 10% EtOAc in Hexane] to afford 5-((5-bromo-6-fluoro-1-((2-(trimethylsilyl)ethoxy)methyl)-1H-pyrrolo[3,2-b]pyridin-2-yl)oxy)-2-methylbenzonitrile as an pale green gum (500 mg, yield: 22%). TLC system: EtOAc/petroleum ether (20:80), R_{f} value: ˜0.3; LCMS (m/z): 476.32 (56+31% purity) (M+H)⁺

### Synthesis of (Z)-5-((5-bromo-6-fluoro-1-((2-(trimethylsilyl)ethoxy)methyl)-1H-pyrrolo[3,2-b]pyridin-2-yl)oxy)-N′-hydroxy-2-methylbenzimidamide

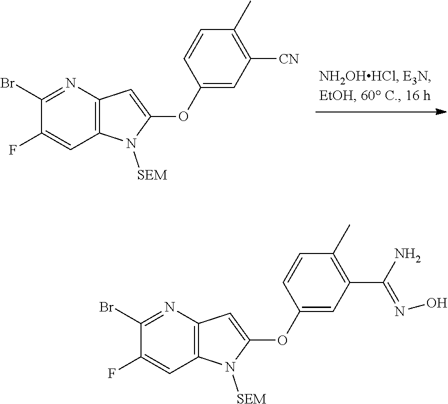 [SMILES: Cc1ccc(Oc2cc3nc(Br)c(F)cc3n2COCC[Si](C)(C)C)cc1C#N ; Cc1ccc(Oc2cc3nc(Br)c(F)cc3n2COCC[Si](C)(C)C)cc1/C(N)=N/O]

To a stirred solution 5-((5-bromo-6-fluoro-1-((2-(trimethylsilyl)ethoxy)methyl)-1H-pyrrolo[3,2-b]pyridin-2-yl)oxy)-2-methylbenzonitrile (500 mg, 1.05 mmol, 1.0 eq) in EtOH (5 mL, 10 Vol) at room temperature, was added Et₃N (0.44 mL, 3.16 mmol, 3.0 eq), NH₂OH·HCl (168 mg, 2.42 mmol, 2.03 eq) and stirred at 60° C. for 16 h. After completion of reaction by TLC, reaction mixture was concentrated under reduced pressure, diluted with ice cold water (20 mL) and extracted with EtOAc (2×50 mL). The combined organic layer was dried over Na₂SO₄, filtered and concentrated under reduced pressure to afford (Z)-5-((5-bromo-6-fluoro-1-((2-(trimethylsilyl)ethoxy)methyl)-1H-pyrrolo[3,2-b]pyridin-2-yl)oxy)-N′-hydroxy-2-methylbenzimidamide as a brown gum (520 mg, crude). TLC system: EtOAc/Hexane (30:70), R_{f} value: ˜0.2; LCMS (m/z): 509.34 (M+2H)⁺

### Synthesis of ethyl (Z)-((5-((5-bromo-6-fluoro-1-((2-(trimethylsilyl)ethoxy)methyl)-1H-pyrrolo[3,2-b]pyridin-2-yl)oxy)-2-methylphenyl)(hydroxyimino)methyl)carbamate

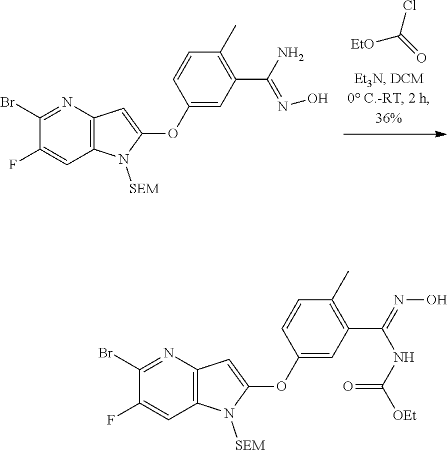 [SMILES: Cc1ccc(Oc2cc3nc(Br)c(F)cc3n2COCC[Si](C)(C)C)cc1/C(N)=N/O ; CCOC(=O)Cl ; CCOC(=O)N/C(=N\O)c1cc(Oc2cc3nc(Br)c(F)cc3n2COCC[Si](C)(C)C)ccc1C]

To a stirred solution of (Z)-5-((5-bromo-6-fluoro-1-((2-(trimethylsilyl)ethoxy)methyl)-1H-pyrrolo[3,2-b]pyridin-2-yl)oxy)-N′-hydroxy-2-methylbenzimidamide (520 mg, 1.02 mmol, 1.0 eq) in DCM (5.2 mL, 10 Vol) at 0° C., was added Et₃N (0.2 mL, 1.33 mmol, 1.3 eq) ethyl carbonochloridate (0.12 mL, 1.22 mmol, 1.2 eq) and stirred at room temperature for 2 h. After completion of reaction by TLC, reaction mixture was diluted with ice cold water (20 mL) extracted with DCM (2×30 mL). The combined organic layer was dried over Na₂SO₄, filtered and concentrated under reduced pressure to afford crude product. The crude product was purified by silica gel (100-200 mesh) column chromatography [elution with 10% EtOAc in Hexane] to afford ethyl (Z)-((5-((5-bromo-6-fluoro-1-((2-(trimethylsilyl)ethoxy)methyl)-1H-pyrrolo[3,2-b]pyridin-2-yl)oxy)-2-methylphenyl)(hydroxyimino)methyl)carbamate as pale brown solid (220 mg, Yield: 36% in 2 steps). TLC system: EtOAc/petroleum ether (30:70), R_{f} value: ˜0.4; LCMS (m/z): 581.35 (M+H)⁺.

### Synthesis of 3-(5-((5-bromo-6-fluoro-1-((2-(trimethylsilyl)ethoxy)methyl)-1H-pyrrolo[3,2-b]pyridin-2-yl)oxy)-2-methylphenyl)-1,2,4-oxadiazol-5(4H)-one

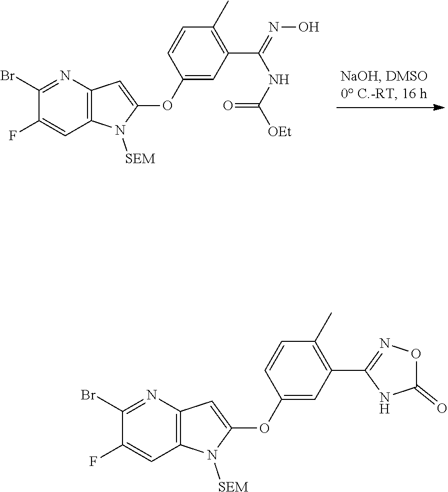 [SMILES: CCOC(=O)N/C(=N\O)c1cc(Oc2cc3nc(Br)c(F)cc3n2COCC[Si](C)(C)C)ccc1C ; Cc1ccc(Oc2cc3nc(Br)c(F)cc3n2COCC[Si](C)(C)C)cc1-c1noc(=O)[nH]1]

To a stirred solution of ethyl (Z)-((5-((5-bromo-6-fluoro-1-((2-(trimethylsilyl)ethoxy)methyl)-1H-pyrrolo[3,2-b]pyridin-2-yl)oxy)-2-methylphenyl)(hydroxyimino)methyl)carbamate (220 mg, 0.38 mmol, 1.0 eq) in DMSO (2.2 mL, 10 Vol) at room temperature, was added NaOH (75 mg, 1.89 mmol, 5.0 eq) and stirred at room temperature for 16 h. After completion of reaction by TLC, reaction mixture was diluted with ice cold water (15 mL), acidified with 2N HCl and extracted with EtOAc (2×30 mL), The combined organic layer washed with ice cold H₂O (20 mL), dried over Na₂SO₄, filtered and concentrated under reduced pressure to afford 3-(5-((5-bromo-6-fluoro-1-((2-(trimethylsilyl)ethoxy)methyl)-1H-pyrrolo[3,2-b]pyridin-2-yl)oxy)-2-methylphenyl)-1,2,4-oxadiazol-5(4H)-one as pale brown gummy (180 mg; Crude). TLC system: EtOAc/petroleum ether (50:50), R_{f} value: ˜0.3; LCMS (m/z): 535.29 (M+H)⁺.

### Synthesis of 3-(5-((6-fluoro-5-(4-(1-hydroxycyclobutyl)phenyl)-1-((2-(trimethylsilyl)ethoxy)methyl)-1H-pyrrolo[3,2-b]pyridin-2-yl)oxy)-2-methylphenyl)-1,2,4-oxadiazol-5(4H)-one

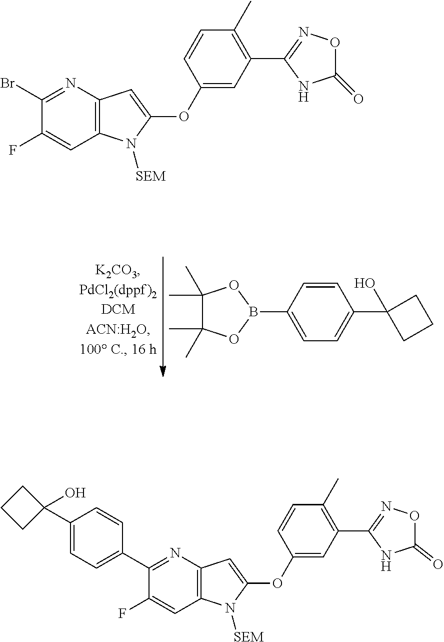 [SMILES: Cc1ccc(Oc2cc3nc(Br)c(F)cc3n2COCC[Si](C)(C)C)cc1-c1noc(=O)[nH]1 ; CC1(C)OB(c2ccc(C3(O)CCC3)cc2)OC1(C)C ; Cc1ccc(Oc2cc3nc(-c4ccc(C5(O)CCC5)cc4)c(F)cc3n2COCC[Si](C)(C)C)cc1-c1noc(=O)[nH]1]

In a sealed tube to a degassed solution of 3-(5-((5-bromo-6-fluoro-1-((2-(trimethylsilyl)ethoxy)methyl)-1H-pyrrolo[3,2-b]pyridin-2-yl)oxy)-2-methylphenyl)-1,2,4-oxadiazol-5(4H)-one (260 mg, 0.48 mmol, 1.0 eq) and 1-(4-(4,4,5,5-tetramethyl-1,3,2-dioxaborolan-2-yl)phenyl)cyclobutan-1-ol (200 mg, 0.73 mmol, 1.5 eq) in ACN:H₂O (4:1, 20 Vol) at room temperature, was added K₂CO₃ (200 mg, 1.46 mmol, 3.0 eq) followed by Pd(dppf)Cl₂·DCM (40 mg, 0.048 mmol, 0.1 eq) and stirred at 100° C. for 16 h. After completion of reaction by TLC, the reaction mixture was diluted with water (10 mL) and extracted with EtOAc (2×30 mL). The combined organic layer was dried over Na₂SO₄, filtered and concentrated under reduced pressure. The crude product was purified by silica gel [100-200] column chromatography [elution with 100% EtOAc] to afford 3-(5-((6-fluoro-5-(4-(1-hydroxycyclobutyl)phenyl)-1-((2-(trimethylsilyl)ethoxy)methyl)-1H-pyrrolo[3,2-b]pyridin-2-yl)oxy)-2-methylphenyl)-1,2,4-oxadiazol-5(4H)-one as a pale brown gum (200 mg, Yield: 87%). TLC system: EtOAc/Hexane (50:50), R_{f} value: ˜0.2; LCMS (m/z): 603.64 (M+H)⁺;

### Synthesis of 3-(5-((6-fluoro-5-(4-(1-hydroxycyclobutyl)phenyl)-1H-pyrrolo[3,2-b]pyridin-2-yl)oxy)-2-methylphenyl)-1,2,4-oxadiazol-5(4H)-one

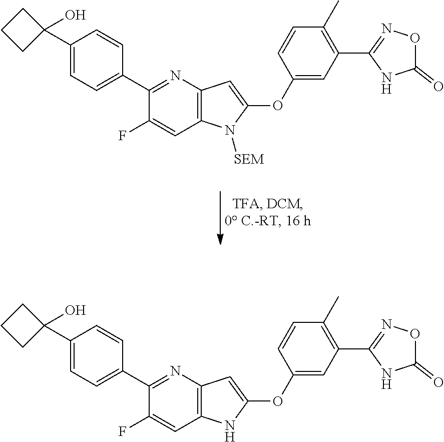 [SMILES: Cc1ccc(Oc2cc3nc(-c4ccc(C5(O)CCC5)cc4)c(F)cc3n2COCC[Si](C)(C)C)cc1-c1noc(=O)[nH]1 ; Cc1ccc(Oc2cc3nc(-c4ccc(C5(O)CCC5)cc4)c(F)cc3[nH]2)cc1-c1noc(=O)[nH]1]

To a stirred solution of 3-(5-((6-fluoro-5-(4-(1-hydroxycyclobutyl)phenyl)-1-((2-(trimethylsilyl)ethoxy)methyl)-1H-pyrrolo[3,2-b]pyridin-2-yl)oxy)-2-methylphenyl)-1,2,4-oxadiazol-5(4H)-one (100 mg, 0.16 mmol, 1.0 eq) in DCM (20 Vol) at 0° C., was added TFA (0.3 mL, 3 Vol) and stirred at room temperature for 16 h. After completion of reaction by TLC, the reaction mixture was concentrated, diluted with ice cold water (10 mL), basified with NaHCO₃ solution and extracted with 10% MeOH/DCM (2×25 mL), The combined organic layer washed with ice cold H₂O (10 mL), dried over Na₂SO₄, filtered and concentrated under reduced pressure. Obtained crude was purified by Prep-HPLC, collected fractions were lyophilized to afford 3-(5-((6-fluoro-5-(4-(1-hydroxycyclobutyl)phenyl)-1H-pyrrolo[3,2-b]pyridin-2-yl)oxy)-2-methylphenyl)-1,2,4-oxadiazol-5(4H)-one as white solid (10 mg; Yield: 13%). TLC system: EtOAc (100:00), R_{f} value: ˜0.2; LCMS (m/z): 473.35 (M+H)⁺; ¹H NMR (400 MHz, DMSO-d₆) δ: 13.0 (brs, 1H), 11.94 (brs, 1H), 7.84-7.82 (m, 2H), 7.63-7.55 (m, 4H), 7.50 (d, J=8.4 Hz, 1H), 7.42 (dd, J=10 Hz, 2.4 Hz, 1H), 5.91 (s, 1H), 5.52 (s, 1H), 2.48 (s, 3H), 2.44-2.40 (m, 2H), 2.33-2.26 (m, 2H), 2.00-1.91 (m, 1H), 1.71-1.68 (m, 1H),

Prep-HPLC condition: Column: ymc C-18 (250*19) mm 5mic; Buffer: MP-(A): 10 mM ABC in WATER MP-(B): ACN: water (80:20); Gradient: (T/% B) 0/30, 15/60, 25/98; Flow Rate: 12 ml/min; Solubility: WATER, ACN, THF

### SYNTHETIC EXAMPLE 52

### Synthesis of Compound II-12

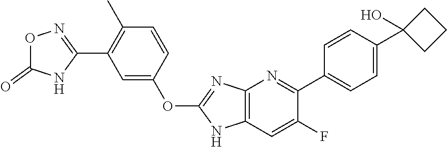 [SMILES: Cc1ccc(Oc2nc3nc(-c4ccc(C5(O)CCC5)cc4)c(F)cc3[nH]2)cc1-c1noc(=O)[nH]1]

### Synthesis of 3-(5-((5-bromo-6-fluoro-1-((2-(trimethylsilyl)ethoxy)methyl)-1H-imidazo[4,5-b]pyridin-2-yl)oxy)-2-methylphenyl)-1,2,4-oxadiazol-5(4H)-one

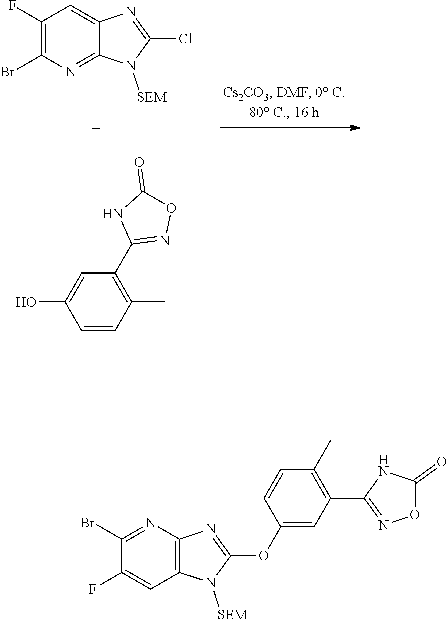 [SMILES: C[Si](C)(C)CCOCn1c(Cl)nc2cc(F)c(Br)nc21 ; Cc1ccc(O)cc1-c1noc(=O)[nH]1 ; Cc1ccc(Oc2nc3nc(Br)c(F)cc3n2COCC[Si](C)(C)C)cc1-c1noc(=O)[nH]1]

To a stirred solution of 6-bromo-2-chloro-5,7-difluoro-1-((2-(trimethylsilyl)ethoxy)methyl)-1H-benzo[d]imidazole (700 mg, 1.85 mmol, 1.0 eq) and 3-(3-hydroxyphenyl)-1,2,4-oxadiazol-5(4H)-one (461 mg, 2.40 mmol, 1.3 eq) in DMF (7.0 mL, 10 Vol) at room temperature, was added Cs₂CO₃ (1.2 g, 3.69 mmol, 2.0 eq) and stirred at 80° C. for 16 h. After completion of reaction by TLC, the reaction mixture was diluted with water (30 mL) and extracted with EtOAc (2×30 mL). The combined organic layer was dried over Na₂SO₄, filtered and concentrated under reduced pressure to afford crude [elution with 30% EtOAc in petroleum ether] to afford 3-(5-((5-bromo-6-fluoro-1-((2-(trimethylsilyl)ethoxy)methyl)-1H-imidazo[4,5-b]pyridin-2-yl)oxy)-2-methylphenyl)-1,2,4-oxadiazol-5(4H)-one as a colorless solid (700 mg, semi-pure). TLC system: EtOAc/Hexane (30:70), R_{f} value: ˜0.4; LCMS (m/z): 536.25 (M+H)⁺.

### Synthesis of 3-(5-((6-fluoro-5-(4-(1-hydroxycyclobutyl)phenyl)-1-((2-(trimethylsilyl)ethoxy)methyl)-1H-imidazo[4,5-b]pyridin-2-yl)oxy)-2-methylphenyl)-1,2,4-oxadiazol-5(4H)-one

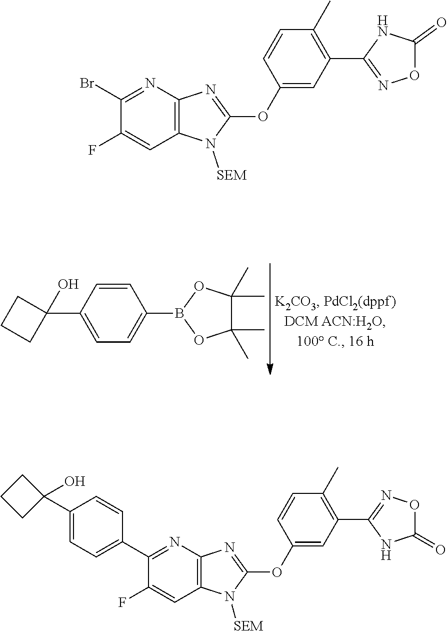

In a sealed tube to a degassed solution of 3-(5-((5-bromo-6-fluoro-1-((2-(trimethylsilyl)ethoxy)methyl)-1H-imidazo[4,5-b]pyridin-2-yl)oxy)-2-methylphenyl)-1,2,4-oxadiazol-5(4H)-one (300 mg, 0.56 mmol, 1.0 eq) and 1-(4-(4,4,5,5-tetramethyl-1,3,2-dioxaborolan-2-yl)phenyl)cyclobutan-1-ol (230 mg, 0.84 mmol, 1.5 eq) in ACN:H₂O (4:1, 20 Vol) at room temperature, was added K₂CO₃ (232 mg, 1.68 mmol, 3.0 eq) followed by Pd(dppf)Cl₂·DCM (46 mg, 0.056 mmol, 0.1 eq) and stirred at 100° C. for 16 h. After completion of reaction by TLC, the reaction mixture was diluted with water (10 mL) and extracted with EtOAc (2×10 mL). The combined organic layer was dried over Na₂SO₄, filtered and concentrated under reduced pressure to afford crude product. The crude product was purified by silica gel [100-200] column chromatography [elution with 50% EtOAc in Hexane] to afford 3-(5-((6-fluoro-5-(4-(1-hydroxycyclobutyl)phenyl)-1-((2-(trimethylsilyl)ethoxy)methyl)-1H-imidazo[4,5-b]pyridin-2-yl)oxy)-2-methylphenyl)-1,2,4-oxadiazol-5(4H)-one as a pale brown solid (250 mg, Yield: 50% in 2 steps). TLC system: EtOAc/Hexane (50:50), R_{f} value: ˜0.4; LCMS (m/z): 604.47 (M+H)⁺.

### Synthesis of 3-(5-((6-fluoro-5-(4-(1-hydroxycyclobutyl)phenyl)-1H-imidazo[4,5-b]pyridin-2-yl)oxy)-2-methylphenyl)-1,2,4-oxadiazol-5(4H)-one

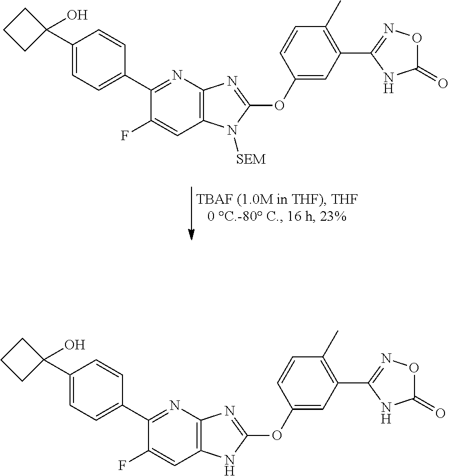 [SMILES: Cc1ccc(Oc2nc3nc(-c4ccc(C5(O)CCC5)cc4)c(F)cc3n2COCC[Si](C)(C)C)cc1-c1noc(=O)[nH]1 ; Cc1ccc(Oc2nc3nc(-c4ccc(C5(O)CCC5)cc4)c(F)cc3[nH]2)cc1-c1noc(=O)[nH]1]

To a stirred solution of 3-(5-((6-fluoro-5-(4-(1-hydroxycyclobutyl)phenyl)-1-((2-(trimethylsilyl)ethoxy)methyl)-1H-imidazo[4,5-b]pyridin-2-yl)oxy)-2-methylphenyl)-1,2,4-oxadiazol-5(4H)-one (250 mg, 0.41 mmol, 1.0 eq) in THF (10 Vol) at 0° C., was added TBAF (2.5 mL, 10 Vol) and stirred at 80° C. for 16 h. After completion of reaction by TLC, the reaction mixture was quenched with NH₄Cl solution (15 mL) and extracted with EtOAc (2×15 mL). The combined organic layer was dried over Na₂SO₄, filtered and concentrated under reduced pressure to afford crude product. The crude product was purified by reverse phase column chromatography [elution with 60% ACN in 0.1% FA in H₂O] to afford semi-pure material which was re-purified by reverse phase column chromatography [elution with 60% ACN in 0.1% FA in H₂O], collected fractions were lyophilized to afford 3-(5-((6-fluoro-5-(4-(1-hydroxycyclobutyl)phenyl)-1H-imidazo[4,5-b]pyridin-2-yl)oxy)-2-methylphenyl)-1,2,4-oxadiazol-5(4H)-one as a white solid (45 mg, Yield: 23%). TLC system: EtOAc/Hexane (100:00), R_{f} value: ˜0.3; LCMS (m/z): 472.23 (M−H)⁻; ¹H NMR (400 MHz, DMSO-d₆) δ: 12.9 (br, 1H), 7.87-7.83 (m, 3H), 7.69-7.67 (m, 1H), 7.62-7.60 (m, 3H), 7.53 (d, J=8.8 Hz, 1H), 5.57 (s, 1H), 2.50 (s, 3H), 2.49-2.40 (m, 2H), 2.34-2.27 (m, 2H), 2.00-1.96 (m, 1H), 1.72-1.68 (m, 1H).

### SYNTHETIC EXAMPLE 53

### Synthesis of Compound II-13

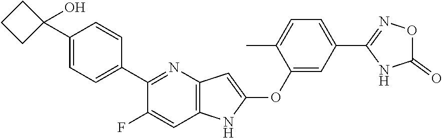 [SMILES: Cc1ccc(-c2noc(=O)[nH]2)cc1Oc1cc2nc(-c3ccc(C4(O)CCC4)cc3)c(F)cc2[nH]1]

### Synthesis of 3-((5-bromo-6-fluoro-1-((2-(trimethylsilyl)ethoxy)methyl)-1H-pyrrolo[3,2-b]pyridin-2-yl)oxy)-4-methylbenzonitrile

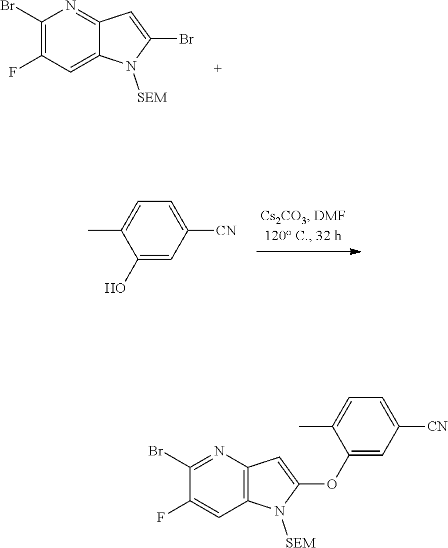

To a stirred solution of 2,5-dibromo-6-fluoro-1-((2-(trimethylsilyl)ethoxy)methyl)-1H-pyrrolo[3,2-b]pyridine (800 mg, 1.89 mmol, 1.0 eq) in DMF (16 mL, 20 Vol) at room temperature, was added Cs₂CO₃ (1.2 g, 3.79 mmol, 2.0 eq) followed by 3-hydroxy-4-methylbenzonitrile (580 mg, 4.36 mmol, 2.3 eq) and stirred at 120° C. for 32 h. After completion of reaction by TLC, the reaction mixture was diluted with water (100 mL) and extracted with EtOAc (2×150 mL). The combined organic layer was dried over Na₂SO₄, filtered and concentrated under reduced pressure. The crude product was purified by silica gel (100-200 mesh) column chromatography [elution with 10% EtOAc in Hexane] to afford 3-((5-bromo-6-fluoro-1-((2-(trimethylsilyl)ethoxy)methyl)-1H-pyrrolo[3,2-b]pyridin-2-yl)oxy)-4-methylbenzonitrile as a brown gum (160 mg, Yield: 18%). TLC system: EtOAc/Hexane (30:70), R_{f} value: ˜0.3; LCMS (m/z): 476.24 (M+H)⁺. Accumulated additional quantities by repeating the reactions.

### Synthesis of (Z)-3-((5-bromo-6-fluoro-1-((2-(trimethylsilyl)ethoxy)methyl)-1H-pyrrolo[3,2-b]pyridin-2-yl)oxy)-N′-hydroxy-4-methylbenzimidamide

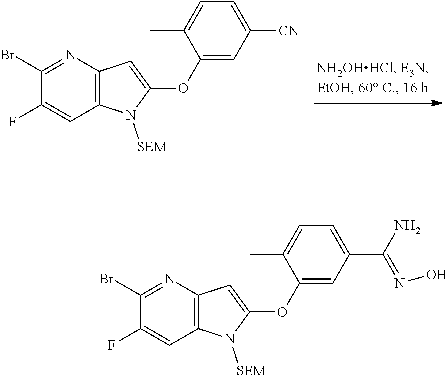 [SMILES: Cc1ccc(C#N)cc1Oc1cc2nc(Br)c(F)cc2n1COCC[Si](C)(C)C ; Cc1ccc(/C(N)=N/O)cc1Oc1cc2nc(Br)c(F)cc2n1COCC[Si](C)(C)C]

To a stirred solution of 3-((5-bromo-6-fluoro-1-((2-(trimethylsilyl)ethoxy)methyl)-1H-pyrrolo[3,2-b]pyridin-2-yl)oxy)-4-methylbenzonitrile (420 mg, 0.88 mmol, 1.0 eq) in EtOH (4.2 mL, 10 Vol) at room temperature, was added Et₃N (0.37 mL, 2.65 mmol, 3.0 eq), NH₂OH·HCl (140 mg, 2.03 mmol, 2.3 eq), and stirred at 60° C. for 16 h. After completion of reaction by TLC, reaction mixture concentrated, diluted with ice cold water (20 mL) and extracted with EtOAc (2×50 mL). The combined organic layer was dried over Na₂SO₄, filtered and concentrated under reduced pressure to afford (Z)-3-((5-bromo-6-fluoro-1-((2-(trimethylsilyl)ethoxy)methyl)-1H-pyrrolo[3,2-b]pyridin-2-yl)oxy)-N′-hydroxy-4-methylbenzimidamide as a brown gummy solid (430 mg, crude). TLC system: EtOAc/Hexane (30:70%), R_{f} value: ˜0.3; LCMS (m/z): 507.17 (M−H)⁺

### Synthesis of ethyl (Z)-((3-((5-bromo-6-fluoro-1-((2-(trimethylsilyl)ethoxy)methyl)-1H-pyrrolo[3,2-b]pyridin-2-yl)oxy)-4-methylphenyl)(hydroxyimino)methyl)carbamate

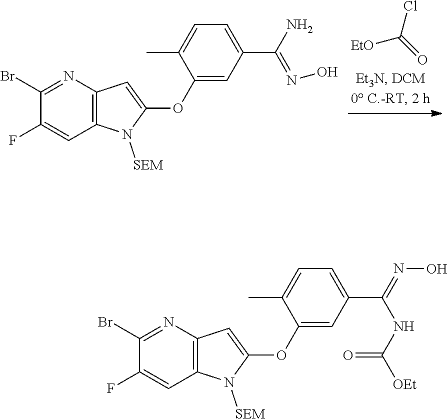 [SMILES: Cc1ccc(/C(N)=N/O)cc1Oc1cc2nc(Br)c(F)cc2n1COCC[Si](C)(C)C ; CCOC(=O)Cl ; CCOC(=O)N/C(=N\O)c1ccc(C)c(Oc2cc3nc(Br)c(F)cc3n2COCC[Si](C)(C)C)c1]

To a stirred solution of (Z)-3-((5-bromo-6-fluoro-1-((2-(trimethylsilyl)ethoxy)methyl)-1H-pyrrolo[3,2-b]pyridin-2-yl)oxy)-N′-hydroxy-4-methylbenzimidamide (430 mg, 0.84 mmol, 1.0 eq) in DCM (4.3 mL, 10 Vol) at 0° C., was added Et₃N (0.15 mL, 1.10 mmol, 1.3 eq), ethyl carbonochloridate (0.1 mL, 1.01 mmol, 1.2 eq) and stirred at room temperature for 2 h. After completion of reaction by TLC, reaction mixture was diluted with ice cold water (20 mL) extracted with DCM (2×30 mL). The combined organic layer was dried over Na₂SO₄, filtered and concentrated under reduced pressure to afford crude product. The crude product was purified by silica gel (100-200 mesh) column chromatography [elution with 30% EtOAc in Hexane] to afford ethyl (Z)-((3-((5-bromo-6-fluoro-1-((2-(trimethylsilyl)ethoxy)methyl)-1H-pyrrolo[3,2-b]pyridin-2-yl)oxy)-4-methylphenyl)(hydroxyimino)methyl)carbamate as a pale brown solid (170 mg, Yield: 34% in 2 steps).TLC system: EtOAc/Petroleum ether (30:70), R_{f} value: ˜0.4; LCMS (m/z): 581.37 (M+H)⁺.

### Synthesis of 3-(3-((5-bromo-6-fluoro-1-((2-(trimethylsilyl)ethoxy)methyl)-1H-pyrrolo[3,2-b]pyridin-2-yl)oxy)-4-methylphenyl)-1,2,4-oxadiazol-5(4H)-one

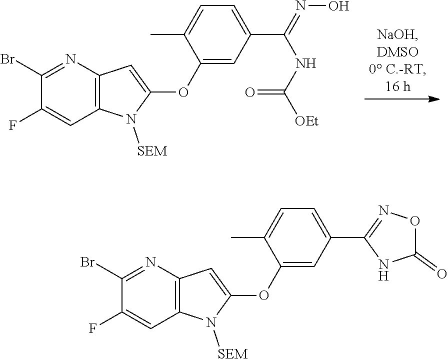 [SMILES: CCOC(=O)N/C(=N\O)c1ccc(C)c(Oc2cc3nc(Br)c(F)cc3n2COCC[Si](C)(C)C)c1 ; Cc1ccc(-c2noc(=O)[nH]2)cc1Oc1cc2nc(Br)c(F)cc2n1COCC[Si](C)(C)C]

To a stirred solution of ethyl (Z)-((3-((5-bromo-6-fluoro-1-((2-(trimethylsilyl)ethoxy)methyl)-1H-pyrrolo[3,2-b]pyridin-2-yl)oxy)-4-methylphenyl)(hydroxyimino)methyl)carbamate (170 mg, 0.29 mmol, 1.0 eq) in DMSO (1.7 mL, 5 Vol) at room temperature, was added NaOH (58 mg, 1.46 mmol, 5.0 eq) and stirred at room temperature for 16 h. After completion of reaction by TLC, reaction mixture was diluted with ice cold water (15 mL), acidified with 2N HCl and extracted with EtOAc (2×30 mL). The combined organic layer washed with ice cold H₂O (20 mL), dried over Na₂SO₄, filtered and concentrated under reduced pressure to afford 3-(3-((5-bromo-6-fluoro-1-((2-(trimethylsilyl)ethoxy)methyl)-1H-pyrrolo[3,2-b]pyridin-2-yl)oxy)-4-methylphenyl)-1,2,4-oxadiazol-5(4H)-one as a pale brown gum (130 mg; Crude). TLC system: EtOAc/Petroleum ether (30:70), R_{f} value: ˜0.2; LCMS (m/z): 535.24 (M+H)⁺.

### Synthesis of 3-(3-((6-fluoro-5-(4-(1-hydroxycyclobutyl)phenyl)-1-((2-(trimethylsilyl)ethoxy)methyl)-1H-pyrrolo[3,2-b]pyridin-2-yl)oxy)-4-methylphenyl)-1,2,4-oxadiazol-5(4H)-one

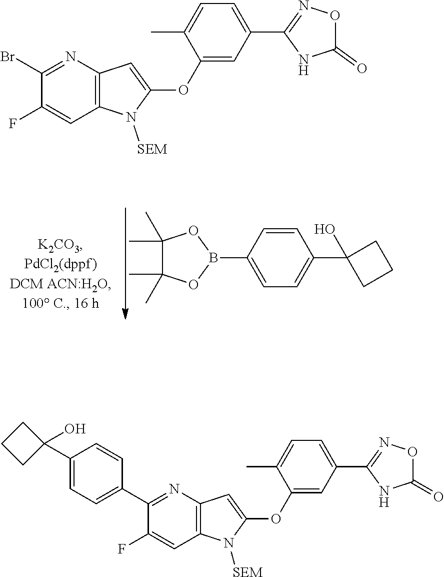 [SMILES: Cc1ccc(-c2noc(=O)[nH]2)cc1Oc1cc2nc(Br)c(F)cc2n1COCC[Si](C)(C)C ; CC1(C)OB(c2ccc(C3(O)CCC3)cc2)OC1(C)C ; Cc1ccc(-c2noc(=O)[nH]2)cc1Oc1cc2nc(-c3ccc(C4(O)CCC4)cc3)c(F)cc2n1COCC[Si](C)(C)C]

In a sealed tube to a degassed solution of 3-(3-((5-bromo-6-fluoro-1-((2-(trimethylsilyl)ethoxy)methyl)-1H-pyrrolo[3,2-b]pyridin-2-yl)oxy)-4-methylphenyl)-1,2,4-oxadiazol-5(4H)-one (130 mg, 0.24 mmol, 1.0 eq) and 1-(4-(4,4,5,5-tetramethyl-1,3,2-dioxaborolan-2-yl)phenyl)cyclobutan-1-ol (100 mg, 0.36 mmol, 1.5 eq) in ACN:H₂O (4:1) (3 mL, 20 Vol) at room temperature, was added K₂CO₃ (100 mg, 0.73 mmol, 3.0 eq) followed by Pd(dppf)Cl₂·DCM (20 mg, 0.024 mmol, 0.1 eq) and stirred at 100° C. for 16 h. After completion of reaction by TLC, the reaction mixture was diluted with water (10 mL) and extracted with EtOAc (2×30 mL). The combined organic layer was dried over Na₂SO₄, filtered and concentrated under reduced pressure to afford crude product. The crude product was purified by silica gel [100-200] column chromatography [elution with 100% EtOAc] to afford 3-(3-((6-fluoro-5-(4-(1-hydroxycyclobutyl)phenyl)-1-((2-(trimethylsilyl)ethoxy)methyl)-1H-pyrrolo[3,2-b]pyridin-2-yl)oxy)-4-methylphenyl)-1,2,4-oxadiazol-5(4H)-one as a pale brown solid (90 mg, Yield: 69%). TLC system: EtOAc/Hexane (50:50), R_{f} value: ˜0.2; LCMS (m/z): 603.55 (M+H)⁺.

### Synthesis of 3-(3-((6-fluoro-5-(4-(1-hydroxycyclobutyl)phenyl)-1H-pyrrolo[3,2-b]pyridin-2-yl)oxy)-4-methylphenyl)-1,2,4-oxadiazol-5(4H)-one

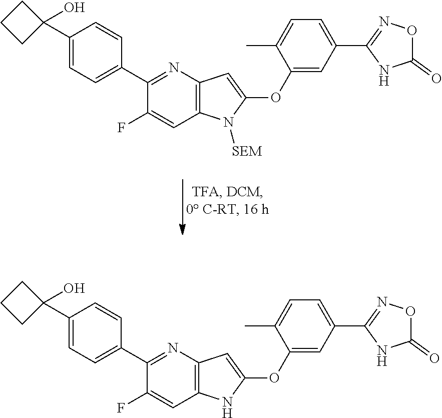 [SMILES: Cc1ccc(-c2noc(=O)[nH]2)cc1Oc1cc2nc(-c3ccc(C4(O)CCC4)cc3)c(F)cc2n1COCC[Si](C)(C)C ; Cc1ccc(-c2noc(=O)[nH]2)cc1Oc1cc2nc(-c3ccc(C4(O)CCC4)cc3)c(F)cc2[nH]1]

To a stirred solution of 3-(3-((6-fluoro-5-(4-(1-hydroxycyclobutyl)phenyl)-1-((2-(trimethylsilyl)ethoxy)methyl)-1H-pyrrolo[3,2-b]pyridin-2-yl)oxy)-4-methylphenyl)-1,2,4-oxadiazol-5(4H)-one (90 mg, 0.15 mmol, 1.0 eq) in DCM (2 mL, 20 Vol) at 0° C., was added TFA (0.3 mL, 3 Vol) and stirred at room temperature for 16 h. After completion of reaction by TLC, the reaction mixture was concentrated, diluted with ice cold water (10 mL), basified with NaHCO₃ solution and extracted with 10% MeOH/DCM (2×25 mL), The combined organic layer washed with ice cold H₂O (10 mL), dried over Na₂SO₄, filtered and concentrated under reduced pressure. The crude product was purified by Prep-HPLC, collected fractions were lyophilized to afford 3-(3-((6-fluoro-5-(4-(1-hydroxycyclobutyl)phenyl)-1H-pyrrolo[3,2-b]pyridin-2-yl)oxy)-4-methylphenyl)-1,2,4-oxadiazol-5(4H)-one as a white solid (8.3 mg; Yield: 12%). TLC system: EtOAc (100%), R_{f} value: ˜0.2; LCMS (m/z): 473.44 (M+H)⁺; ¹H NMR (400 MHz, DMSO-d₆) δ: 12.95 (brs, 1H), 11.98 (s, 1H), 7.85-7.82 (m, 2H), 7.65-7.55 (m, 6H), 5.81 (s, 1H), 5.53 (s, 1H), 2.46-2.40 (m, 2H), 2.36 (s, 3H), 2.33-2.26 (m, 2H), 1.96-1.91 (m, 1H), 1.71-1.66 (m, 1H).

### SYNTHETIC EXAMPLE 54

### Synthesis of Compound II-14

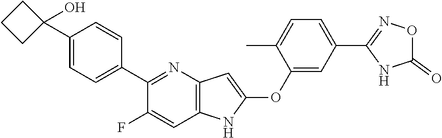 [SMILES: Cc1ccc(-c2noc(=O)[nH]2)cc1Oc1cc2nc(-c3ccc(C4(O)CCC4)cc3)c(F)cc2[nH]1]

### Synthesis of 3-(3-((5-bromo-6-fluoro-1-((2-(trimethylsilyl)ethoxy)methyl)-1H-imidazo[4,5-b]pyridin-2-yl)oxy)-4-methylphenyl)-1,2,4-oxadiazol-5(4H)-one

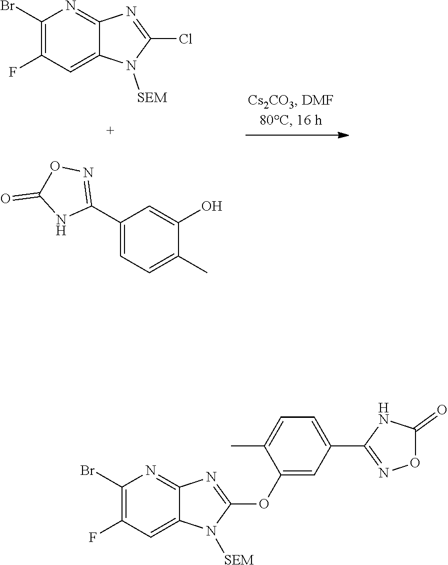

To a stirred solution of 5-bromo-2-chloro-6-fluoro-1-((2-(trimethylsilyl)ethoxy)methyl)-1H-imidazo[4,5-b]pyridine (500 mg, 1.32 mmol, 1.0 eq) and 3-(3-hydroxy-4-methylphenyl)-1,2,4-oxadiazol-5(4H)-one (329 mg, 1.72 mmol, 1.3 eq) in DMF (5 mL, 10 vol) at room temperature, was added Cs₂CO₃ (860 mg, 2.64 mmol, 2.0 eq), and stirred at 80° C. for 16 h. After completion of reaction by TLC, the reaction mixture was diluted with water (80 mL) and extracted with EtOAc (2×150 mL). The combined organic layer was dried over Na₂SO₄, filtered and concentrated under reduced pressure to afford crude compound. Purified by silica column (100-200 mesh) chromatography [elution with 50% EtOAc in petroleum ether] to afford 3-(3-((5-bromo-6-fluoro-1-((2-(trimethylsilyl)ethoxy)methyl)-1H-imidazo[4,5-b]pyridin-2-yl)oxy)-4-methylphenyl)-1,2,4-oxadiazol-5(4H)-one as a brown solid (400 mg, Yield: 56%). TLC system: EtOAc/Hexane (50:50), R_{f} value: ˜0.3; LCMS (m/z): 534.11 (M−H)⁻.

### Synthesis of 3-(3-((6-fluoro-5-(4-(1-hydroxycyclobutyl)phenyl)-1-((2-(trimethylsilyl)ethoxy)methyl)-1H-imidazo[4,5-b]pyridin-2-yl)oxy)-4-methylphenyl)-1,2,4-oxadiazol-5(4H)-one

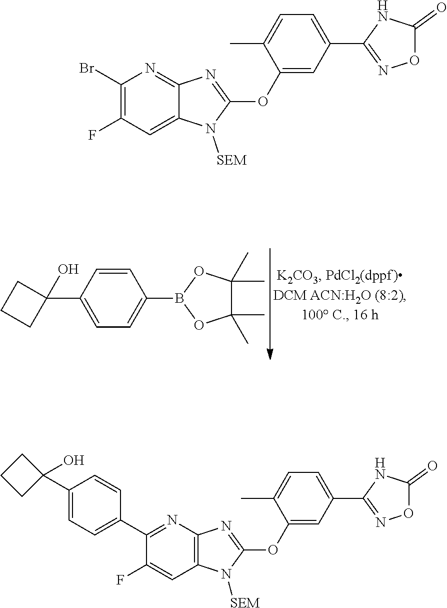 [SMILES: Cc1ccc(-c2noc(=O)[nH]2)cc1Oc1nc2nc(Br)c(F)cc2n1COCC[Si](C)(C)C ; CC1(C)OB(c2ccc(C3(O)CCC3)cc2)OC1(C)C ; Cc1ccc(-c2noc(=O)[nH]2)cc1Oc1nc2nc(-c3ccc(C4(O)CCC4)cc3)c(F)cc2n1COCC[Si](C)(C)C]

In a sealed tube, to a degassed solution of 3-(3-((5-bromo-6-fluoro-1-((2-(trimethylsilyl)ethoxy)methyl)-1H-imidazo[4,5-b]pyridin-2-yl)oxy)-4-methylphenyl)-1,2,4-oxadiazol-5(4H)-one (150 mg, 0.28 mmol, 1.0 eq) and 1-(4-(4,4,5,5-tetramethyl-1,3,2-dioxaborolan-2-yl)phenyl)cyclobutan-1-ol (115 mg, 0.42 mmol, 1.5 eq) in ACN:H₂O (8:2) (3 mL, 20 vol) at room temperature, was added K₂CO₃ (116 mg, 0.84 mmol, 3.0 eq) followed by PdCl₂(dppf)·DCM (23 mg, 0.028 mmol, 0.1 eq) and stirred at 100° C. for 16 h. After completion of reaction by TLC, the reaction mixture was diluted with water (20 mL) and extracted with EtOAc (2×100 mL). The combined organic layer was dried over Na₂SO₄, filtered and concentrated under reduced pressure. Obtained crude was purified by silica column (100-200 mesh) chromatography to afford 3-(3-((6-fluoro-5-(4-(1-hydroxycyclobutyl)phenyl)-1-((2-(trimethylsilyl)ethoxy)methyl)-1H-imidazo[4,5-b]pyridin-2-yl)oxy)-4-methylphenyl)-1,2,4-oxadiazol-5(4H)-one as colorless gummy liquid (140 mg, Yield: 83%). TLC system: EtOAc/Hexane (50:50), R_{f} value: ˜0.2; LCMS (m/z): 604.52 (M+H)⁺.

### Synthesis of 3-(3-((6-fluoro-5-(4-(1-hydroxycyclobutyl)phenyl)-1H-imidazo[4,5-b]pyridin-2-yl)oxy)-4-methylphenyl)-1,2,4-oxadiazol-5(4H)-one

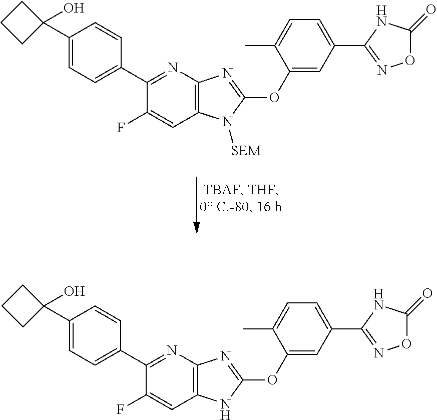 [SMILES: Cc1ccc(-c2noc(=O)[nH]2)cc1Oc1nc2nc(-c3ccc(C4(O)CCC4)cc3)c(F)cc2n1COCC[Si](C)(C)C ; Cc1ccc(-c2noc(=O)[nH]2)cc1Oc1nc2nc(-c3ccc(C4(O)CCC4)cc3)c(F)cc2[nH]1]

To a stirred solution of 3-(3-((5-(4-((dimethyl(oxo)-16-sulfaneylidene)amino)phenyl)-6-fluoro-1-((2-(trimethylsilyl)ethoxy)methyl)-1H-imidazo[4,5-b]pyridin-2-yl)oxy)-4-methylphenyl)-1,2,4-oxadiazol-5(4H)-one (140 mg, 0.23 mmol, 1.0 eq) in THF (1.4 mL, 10 vol) at 0° C., was added TBAF (1.4 mL, 10 vol) and stirred at 80° C. for 16 h. After completion of reaction by TLC, the reaction mixture concentrated under reduced pressure and washed with pentane (20 mL) to afford crude compound. Purified by Prep-HPLC, collected fractions were lyophilized to afford 3-(3-((6-fluoro-5-(4-(1-hydroxycyclobutyl)phenyl)-1H-imidazo[4,5-b]pyridin-2-yl)oxy)-4-methylphenyl)-1,2,4-oxadiazol-5(4H)-one as off white solid (13 mg, Yield: 10%). TLC system: EtOAc (100%), R_{f} value: ˜0.1; LCMS (m/z): 472.23 (M−H)⁻; ¹H NMR (400 MHz, DMSO-d₆) δ: 13.44 (brs, 1H), 12.95 (s, 1H), 7.86-7.82 (m, 4H), 7.70 (dd, J=6.4 Hz, J=1.6 Hz, 1H), 7.61 (d, J=8.0 Hz, 3H), 5.57 (brs, 1H), 2.49-2.40 (m, 2H), 2.33-2.29 (m, 5H), 1.99-1.90 (m, 1H), 1.74-1.65 (m, 1H).

### SYNTHETIC EXAMPLE 55

### Synthesis of Compound I-73

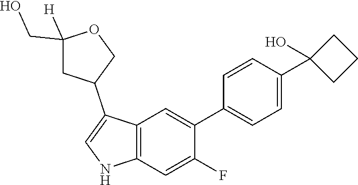 [SMILES: OCC1CC(c2c[nH]c3cc(F)c(-c4ccc(C5(O)CCC5)cc4)cc23)CO1]

### Synthesis of ethyl 4-(((trifluoromethyl)sulfonyl)oxy)-2,5-dihydrofuran-2-carboxylate

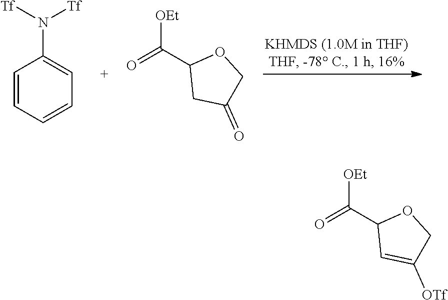

To a stirred solution of ethyl 4-oxotetrahydrofuran-2-carboxylate (500 mg, 3.16 mmol, 1.0 eq) in dry THF (20 Vol) at −78° C., was added KHMDS (1.0 M in THF) (4.1 mL, 4.11 mmol, 1.3 eq) and stirred for 30 min. After that a solution of 1,1,1-trifluoro-N-phenyl-N-((trifluoromethyl)sulfonyl)methanesulfonamide (1.12 g, 3.16 mmol, 1 eq) in THF (10 Vol) was added to reaction mixture at same temperature and stirred for 30 min. After completion of reaction by TLC, the reaction mixture was quenched with NH₄Cl solution (30 mL) and extracted with EtOAc (2×30 mL). The combined organic layer was dried over Na₂SO₄, filtered and concentrated under reduced pressure to afford crude product. Three more batches of same scale were repeated and combined crude material purified by silica gel [100-200] column chromatography [elution with 40% DCM in Hexane] to afford ethyl 4-(((trifluoromethyl)sulfonyl)oxy)-2,5-dihydrofuran-2-carboxylate as a yellow liquid (600 mg, Yield: 16%). TLC system: EtOAc/Hexane (20:80) (KmnO₄ active), R_{f} value: ˜0.5; ¹H NMR (400 MHz, CDCl₃) δ: 5.89-5.87 (m, 1H), 5.30-5.29 (m, 1H), 4.88-4.83 (m, 1H), 4.72-4.68 (m, 1H), 4.29-4.20 (m, 2H), 1.31 (t, J=7.2 Hz, 3H).

### Synthesis of 1-(4-(6-fluoro-1H-indol-5-yl)phenyl)cyclobutan-1-ol

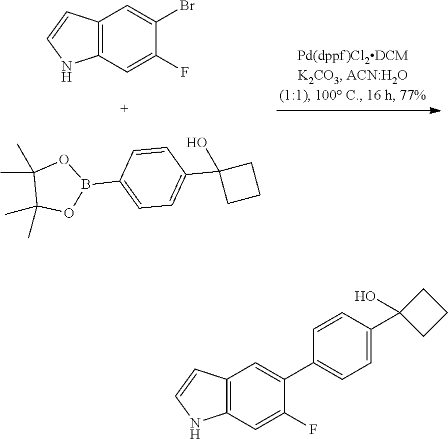 [SMILES: Fc1cc2[nH]ccc2cc1Br ; CC1(C)OB(c2ccc(C3(O)CCC3)cc2)OC1(C)C ; OC1(c2ccc(-c3cc4cc[nH]c4cc3F)cc2)CCC1]

In a sealed tube to a degassed solution of 5-bromo-6-fluoro-1H-indole (4.0 g, 18.7 mmol, 1.0 eq) and 1-(4-(4,4,5,5-tetramethyl-1,3,2-dioxaborolan-2-yl)phenyl)cyclobutan-1-ol (6.14 g, 22.4 mmol, 1.2 eq) in ACN:H₂O (1:1, 40 mL) at room temperature, was added K₂CO₃ (7.73 g, 56.1 mmol, 3.0 eq) followed by Pd(dppf)Cl₂·DCM (1.52 g, 1.87 mmol, 0.1 eq) and stirred at 100° C. for 16 h. After completion of reaction by TLC, the reaction mixture was diluted with water (30 mL) and extracted with EtOAc (2×80 mL). The combined organic layer was dried over Na₂SO₄, filtered and concentrated under reduced pressure. The crude product was purified by silica gel [100-200] column chromatography [elution with 15% EtOAc in petroleum ether] to afford 1-(4-(6-fluoro-1H-indol-5-yl)phenyl)cyclobutan-1-ol as orange gummy (4 g, Yield: 77%). TLC system: EtOAc/Hexane (30:80), R_{f} value: ˜0.2; ¹H NMR (400 MHz, CDCl₃) δ: 8.21 (bs, 1H), 7.65 (d, J=7.6 Hz, 1H), 7.62-7.51 (m, 4H), 7.23-7.17 (m, 2H), 6.58 (s, 1H), 2.64-2.60 (m, 2H), 2.45-2.38 (m, 2H), 2.09-2.01 (m, 1H), 1.77-1.67 (m, 1H).

### Synthesis of 1-(4-(3-bromo-6-fluoro-1H-indol-5-yl)phenyl)cyclobutan-1-ol

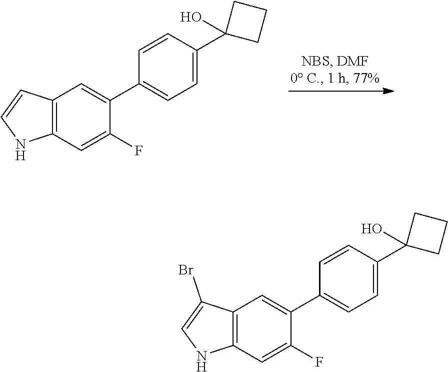 [SMILES: OC1(c2ccc(-c3cc4cc[nH]c4cc3F)cc2)CCC1 ; OC1(c2ccc(-c3cc4c(Br)c[nH]c4cc3F)cc2)CCC1]

To a stirred solution of 1-(4-(6-fluoro-1H-indol-5-yl)phenyl)cyclobutan-1-ol (4.0 g, 14.23 mmol, 1.0 eq) in DMF (10 Vol) at 0° C., was added solution of NBS (2.53 g, 14.23 mmol, 1.0 eq) in DMF (10 Vol) and stirred at 0° C. for 1 h. After completion of reaction by TLC, the reaction mixture was diluted with water (30 mL) and extracted with EtOAc (2×50 mL). The combined organic layer was dried over Na₂SO₄, filtered and concentrated under reduced pressure to afford crude product. The crude product was purified by silica gel [100-200] column chromatography [elution with 30% EtOAc in petroleum ether] to afford 1-(4-(3-bromo-6-fluoro-1H-indol-5-yl)phenyl)cyclobutan-1-ol as yellow gum (4.0 g, Yield: 77%). TLC system: EtOAc/Hexane (50:50), R_{f} value: ˜0.5; LCMS (m/z): 358.10 (M−H)⁻; ¹H NMR (400 MHz, CDCl₃) δ: 8.26 (bs, 1H), 7.64-7.58 (m, 5H), 7.20 (d, J=10.8 Hz, 1H), 2.67-2.60 (m, 2H), 2.46-2.39 (m, 2H), 2.08-2.03 (m, 1H), 1.78-1.71 (m, 1H).

### Synthesis of 1-(4-(3-bromo-6-fluoro-1-tosyl-1H-indol-5-yl)phenyl)cyclobutan-1-ol

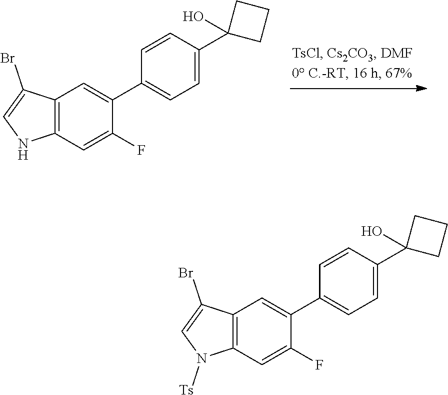 [SMILES: OC1(c2ccc(-c3cc4c(Br)c[nH]c4cc3F)cc2)CCC1 ; Cc1ccc(S(=O)(=O)n2cc(Br)c3cc(-c4ccc(C5(O)CCC5)cc4)c(F)cc32)cc1]

To a stirred solution of 1-(4-(3-bromo-6-fluoro-1H-indol-5-yl)phenyl)cyclobutan-1-ol (4.0 g, 11.23 mmol, 1.0 eq) in DMF (40 Vol) at 0° C., was added Cs₂CO₃ (7.26 g, 22.3 mmol, 2.0 eq) followed by Ts-Cl (4.2 g, 22.3 mmol, 2.0 eq) and stirred at room temperature for 16 h. After completion of reaction by TLC, the reaction mixture was diluted with water (50 mL) and extracted with EtOAc (2×100 mL). The combined organic layer was dried over Na₂SO₄, filtered and concentrated under reduced pressure to afford crude product. The crude product was purified by silica gel [100-200] column chromatography [elution with 30% EtOAc in petroleum ether] to afford 1-(4-(3-bromo-6-fluoro-1-tosyl-1H-indol-5-yl)phenyl)cyclobutan-1-ol as a brown solid (3.5 g, Yield: 67%). TLC system: EtOAc/Hexane (30:70), R_{f} value: ˜0.5; ¹H NMR (400 MHz, CDCl₃) δ: 7.83-7.80 (m, 3H), 7.61-7.58 (m, 3H), 7.56 (dd, J=1.6 Hz, 8.4 Hz, 2H), 7.49 (d, J=7.6 Hz, 1H), 7.29 (d, J=8 Hz, 2H), 2.65-2.58 (m, 2H), 2.45-2.37 (m, 5H), 2.09-2.03 (m, 1H), 1.77-1.70 (m, 1H).

### Synthesis of 1-(4-(6-fluoro-3-(4,4,5,5-tetramethyl-1,3,2-dioxaborolan-2-yl)-1-tosyl-1H-indol-5-yl)phenyl)cyclobutan-1-ol

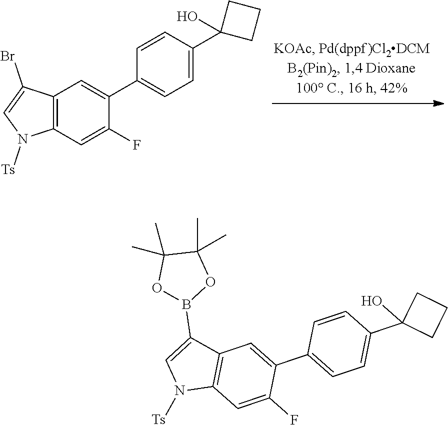 [SMILES: Cc1ccc(S(=O)(=O)n2cc(Br)c3cc(-c4ccc(C5(O)CCC5)cc4)c(F)cc32)cc1 ; Cc1ccc(S(=O)(=O)n2cc(B3OC(C)(C)C(C)(C)O3)c3cc(-c4ccc(C5(O)CCC5)cc4)c(F)cc32)cc1]

In a sealed tube to a degassed solution of 1-(4-(3-bromo-6-fluoro-1-tosyl-1H-indol-5-yl)phenyl)cyclobutan-1-ol (2.2 g, 4.29 mmol, 1.0 eq) and B₂Pin₂ (2.18 g, 8.57 mmol, 2.0 eq) in Dioxane (20 Vol, 20 mL) at room temperature, was added KOAc (1.26 g, 12.87 mmol, 3.0 eq) followed by Pd(dppf)Cl₂·DCM (350 mg, 0.43 mmol, 0.1 eq) and stirred at 100° C. for 16 h. After completion of reaction by TLC, the reaction mixture was filtered through a pad of diatomaceous earth (e.g., Celite®), washed with EtOAc (50 mL), collected filtrate was concentrated under reduced pressure. The crude product was purified by reverse phase column chromatography [elution with 95% ACN in 0.1% FA in H₂O] to afford 1-(4-(6-fluoro-3-(4,4,5,5-tetramethyl-1,3,2-dioxaborolan-2-yl)-1-tosyl-1H-indol-5-yl)phenyl)cyclobutan-1-ol as an off white solid (1.0 g, Yield: 42%). TLC system: EtOAc/Hexane (20:80), LCMS (m/z): 544.32 (M−18+H)⁺;

### Synthesis of ethyl 4-(6-fluoro-5-(4-(1-hydroxycyclobutyl)phenyl)-1-tosyl-1H-indol-3-yl)-2,5-dihydrofuran-2-carboxylate

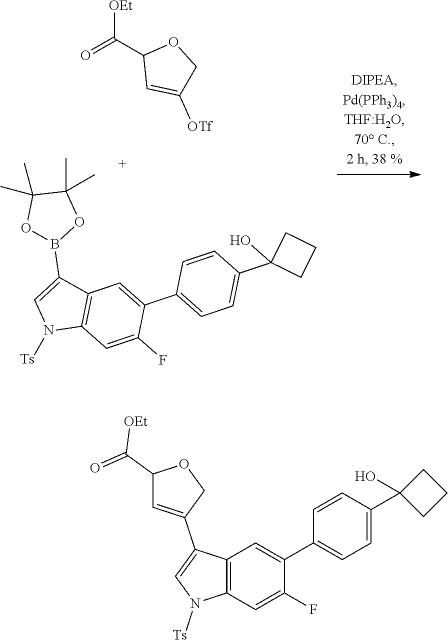 [SMILES: CCOC(=O)C1C=C(OS(=O)(=O)C(F)(F)F)CO1 ; Cc1ccc(S(=O)(=O)n2cc(B3OC(C)(C)C(C)(C)O3)c3cc(-c4ccc(C5(O)CCC5)cc4)c(F)cc32)cc1 ; CCOC(=O)C1C=C(c2cn(S(=O)(=O)c3ccc(C)cc3)c3cc(F)c(-c4ccc(C5(O)CCC5)cc4)cc23)CO1]

In a sealed tube to a degassed solution of ethyl 4-(((trifluoromethyl)sulfonyl)oxy)-2,5-dihydrofuran-2-carboxylate (600 mg, 2.07 mmol, 1.0 eq) and 1-(4-(6-fluoro-3-(4,4,5,5-tetramethyl-1,3,2-dioxaborolan-2-yl)-1-tosyl-1H-indol-5-yl)phenyl)cyclobutan-1-ol (697 mg, 1.24 mmol, 0.6 eq) in THF:H₂O (1:1, 6 mL) at room temperature, was added DIPEA (1.8 mL, 10.35 mmol, 5.0 eq) followed by Pd(PPh₃)₄ (120 mg, 0.1 mmol, 0.05 eq) and stirred at 70° C. for 2 h. After completion of reaction by TLC, the reaction mixture was filtered through a pad of diatomaceous earth (e.g., Celite®), washed with EtOAc (30 mL), collected filtrate was concentrated under reduced pressure. The crude product was purified by silica gel [100-200] column chromatography [elution with 30% EtOAc in petroleum ether] to afford ethyl 4-(6-fluoro-5-(4-(1-hydroxycyclobutyl)phenyl)-1-tosyl-1H-indol-3-yl)-2,5-dihydrofuran-2-carboxylate as an off white solid (450 mg, Yield: 38%). TLC system: EtOAc/Hexane (50:50), R_{f} value: ˜0.5; LCMS (m/z): 574.25 (M−H)⁻

### Synthesis of ethyl 4-(6-fluoro-5-(4-(1-hydroxycyclobutyl)phenyl)-1-tosyl-1H-indol-3-yl)tetrahydrofuran-2-carboxylate

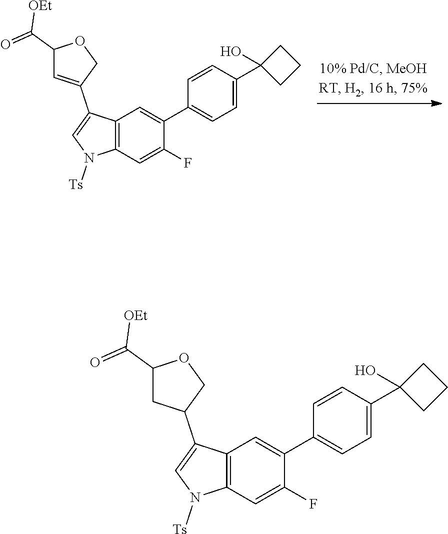 [SMILES: CCOC(=O)C1C=C(c2cn(S(=O)(=O)c3ccc(C)cc3)c3cc(F)c(-c4ccc(C5(O)CCC5)cc4)cc23)CO1 ; CCOC(=O)C1CC(c2cn(S(=O)(=O)c3ccc(C)cc3)c3cc(F)c(-c4ccc(C5(O)CCC5)cc4)cc23)CO1]

To a stirred solution of ethyl 4-(6-fluoro-5-(4-(1-hydroxycyclobutyl)phenyl)-1- to syl-1H-indol-3-yl)-2,5-dihydrofuran-2-carboxylate (450 mg, 0.78 mmol, 1.0 eq) in MeOH (4.5 mL) at room temperature, was added 10% Pd/C (130 mg, 30% Wt/Wt) and stirred for 16 h under H₂ atmosphere. After completion of reaction by TLC, the reaction mixture was filtered through a pad of diatomaceous earth (e.g., Celite®), washed with DCM (30 mL), collected filtrate was concentrated under reduced pressure. The crude product was purified by silica gel [100-200] column chromatography [elution with 30% EtOAc in petroleum ether] to afford ethyl 4-(6-fluoro-5-(4-(1-hydroxycyclobutyl)phenyl)-1-tosyl-1H-indol-3-yl)tetrahydrofuran-2-carboxylate as a white solid (340 mg, Yield: 75%). TLC system: EtOAc/Hexane (50:50), R_{f} value: ˜0.4; LCMS (m/z): 560.38 (M−OH)+; ¹H NMR (400 MHz, CDCl₃) δ: 7.81-7.78 (m, 3H), 7.60-7.58 (m, 2H), 7.54-7.49 (m, 3H), 7.39 (m, 1H), 7.30-7.27 (m, 2H), 4.62 (t, J=8.0 Hz, 1H), 4.33 (t, J=8.0 Hz, 1H), 4.22 (q, J=6.4 Hz, 2H), 3.98 (t, J=8.8 Hz, 1H), 3.63-3.60 (m, 1H), 2.80-2.77 (m, 1H), 2.63-2.58 (m, 2H), 2.45-2.38 (m, 5H), 2.22-2.19 (m, 1H), 2.04-2.02 (m, 1H), 1.75-1.73 (m, 1H), 1.26 (s, 3H).

### Synthesis of 1-(4-(6-fluoro-3-(5-(hydroxymethyl)tetrahydrofuran-3-yl)-1H-indol-5-yl)phenyl)cyclobutan-1-ol

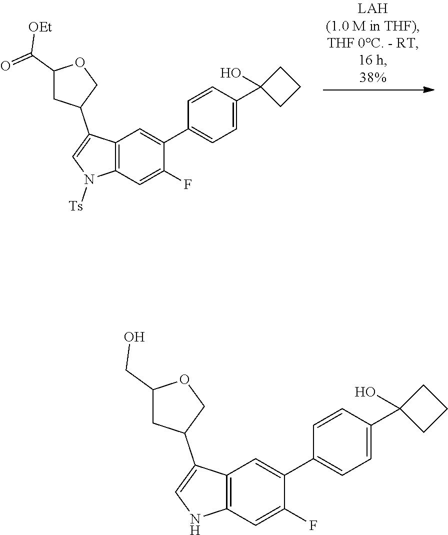 [SMILES: CCOC(=O)C1CC(c2cn(S(=O)(=O)c3ccc(C)cc3)c3cc(F)c(-c4ccc(C5(O)CCC5)cc4)cc23)CO1 ; OCC1CC(c2c[nH]c3cc(F)c(-c4ccc(C5(O)CCC5)cc4)cc23)CO1]

To a stirred solution of ethyl 4-(6-fluoro-5-(4-(1-hydroxycyclobutyl)phenyl)-1- to syl-1H-indol-3-yl)tetrahydrofuran-2-carboxylate (200 mg, 0.35 mmol, 1.0 eq) in THF (2.0 mL) at 0° C., was added LAH (2.0 M in THF) (0.52 mL, 1.04 mmol, 3.0 eq) and stirred at room temperature for 16 h. After completion of reaction by TLC, the reaction mixture was quenched with NH₄Cl solution (15 mL) and EtOAc (10 mL). Solution was filtered through a pad of diatomaceous earth (e.g., Celite®), washed with EtOAc (15 mL) and extracted with H₂O (2×5 mL). The combined organic layer was dried over Na₂SO₄, filtered and concentrated under reduced pressure to afford crude product. The crude product was purified by Prep-HPLC. Collected fractions were lyophilized to afford 1-(4-(6-fluoro-3-(5-(hydroxymethyl)tetrahydrofuran-3-yl)-1H-indol-5-yl)phenyl)cyclobutan-1-ol as white solid (50 mg, Yield: 38%). TLC system: EtOAc/Hexane (100:00), R_{f} value: ˜0.3; LCMS (m/z): 380.27 (M−H)⁻; ¹H NMR (400 MHz, DMSO-d₆) δ: 10.96 (s, 1H), 7.63 (d, J=8.0 Hz, 1H), 7.57-7.51 (m, 4H), 7.23-7.20 (m, 2H), 5.52 (s, 1H), 4.74 (t, J=5.6 Hz, 1H), 4.12 (t, J=7.2 Hz, 1H), 4.04-4.03 (m, 1H), 3.73-3.64 (m, 2H), 3.50-3.40 (m, 2H), 2.47-2.40 (m, 2H), 2.38-2.29 (m, 3H), 1.87-1.84 (m, 2H), 1.69-1.67 (m, 1H).

Prep-HPLC condition: Column Gemini C-18 (250*21.2) mm 5mic Buffer MP-(A): 0.1% FA in WATER MP-(B): ACN:WATER (80:20) Gradient (T/% B) 0/40, 15/70, 20/95, 25/98 Flow Rate 13 ml/min Solubility ACN, water, THF

### SYNTHETIC EXAMPLE 56

### Synthesis of Compound II-17

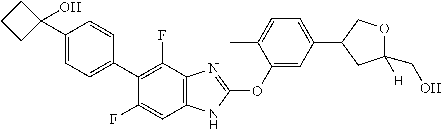 [SMILES: Cc1ccc(C2COC(CO)C2)cc1Oc1nc2c(F)c(-c3ccc(C4(O)CCC4)cc3)c(F)cc2[nH]1]

### Synthesis of 5-bromo-2-methylphenol

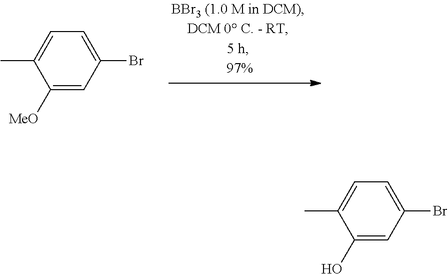

To a stirred solution of 4-bromo-2-methoxy-1-methylbenzene (5 g, 24.87 mmol, 1.0 eq) in dry DCM (10 Vol) at 0° C., was added BBr₃ (1.0 M in THF) (49.7 mL, 49.75 mmol, 2.0 eq) and stirred at room temperature for 5 h. After completion of reaction by TLC, the reaction mixture was quenched with NaHCO₃ solution (50 mL) and extracted with DCM (2×50 mL). The combined organic layer was dried over Na₂SO₄, filtered and concentrated under reduced pressure to afford 5-bromo-2-methylphenol as a brown solid (4.5 g, Yield: 97%). TLC system: EtOAc/Hexane (5:95), R_{f} value: ˜0.2; LCMS (m/z): 185.03 (M−H)⁻; ¹H NMR (400 MHz, CDCl₃) δ: 6.97-6.96 (m, 2H), 6.93 (s, 1H), 4.98 (bs, 1H), 2.18 (s, 3H).

### Synthesis of 2-(benzyloxy)-4-bromo-1-methylbenzene

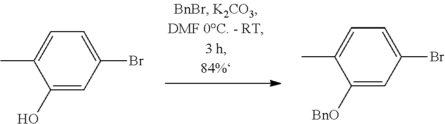

To a stirred solution of 5-bromo-2-methylphenol (4.5 g, 24.19 mmol, 1.0 eq) in DMF (10 Vol) at 0° C., was added K₂CO₃ (6.67 g, 48.38 mmol, 2.0 eq) followed by BnBr (5.0 g, 29.03 mmol, 1.2 eq) and stirred at room temperature for 3 h. After completion of reaction by TLC, the reaction mixture was diluted with water (50 mL) and extracted with EtOAc (2×50 mL). The combined organic layer was dried over Na₂SO₄, filtered and concentrated under reduced pressure to afford crude product. The crude product was purified by silica gel [60-120] column chromatography [elution with 100% petroleum ether] to afford 2-(benzyloxy)-4-bromo-1-methylbenzene as a colorless liquid (5.6 g, Yield: 84%). TLC system: EtOAc/Hexane (00:100), Re value: ˜0.6; ¹H NMR (400 MHz, CDCl₃) δ: 7.45-7.38 (m, 4H), 7.36-7.32 (m, 1H), 7.03-6.99 (m, 3H), 5.05 (s, 2H), 2.21 (s, 3H).

### Synthesis of 2-(3-(benzyloxy)-4-methylphenyl)-4,4,5,5-tetramethyl-1,3,2-dioxaborolane

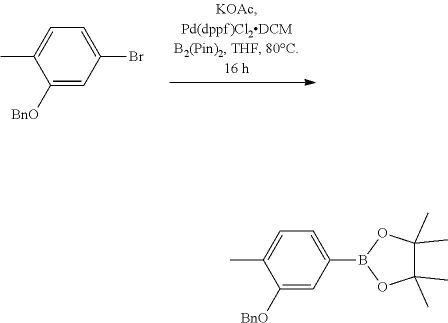

In a sealed tube to a degassed solution of 2-(benzyloxy)-4-bromo-1-methylbenzene (5.2 g, 23.01 mmol, 1.0 eq) and B₂Pin₂ (7.0 g, 27.61 mmol, 1.2 eq) in THF (10 Vol, 52 mL) at room temperature, was added KOAc (6.8 g, 69.02 mmol, 3.0 eq) followed by Pd(dppf)Cl₂·DCM (1.9 g, 2.30 mmol, 0.1 eq) and stirred at 80° C. for 16 h. After completion of reaction by TLC, the reaction mixture was diluted with water (50 mL) and extracted with EtOAc (2×50 mL). The combined organic layer was dried over Na₂SO₄, filtered and concentrated under reduced pressure to afford crude product. The crude product was purified by silica gel [100-200] column chromatography [elution with 10% EtOAc in petroleum ether] to afford 2-(3-(benzyloxy)-4-methylphenyl)-4,4,5,5-tetramethyl-1,3,2-dioxaborolane as an off white solid (4.0 g, semi-pure). TLC system: EtOAc/Hexane (10:90), R_{f} value: ˜0.4; ¹H NMR (400 MHz, CDCl₃) δ: 7.49-7.46 (m, 2H), 7.42-7.32 (m, 4H), 7.23-7.17 (m, 2H), 5.12 (s, 2H), 2.30 (s, 3H), 1.35 (s, 12H), semi-pure sample.

### Synthesis of ethyl 4-(3-(benzyloxy)-4-methylphenyl)-2,5-dihydrofuran-2-carboxylate

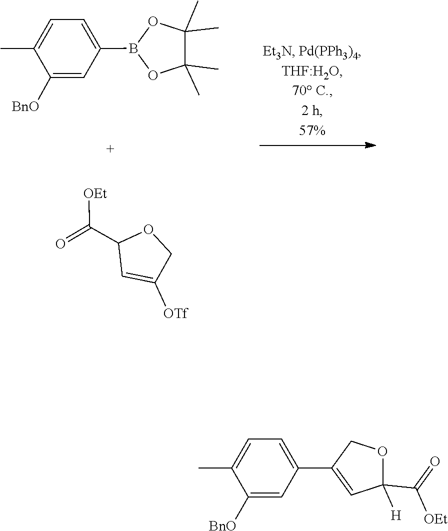

In a sealed tube to a degassed solution of ethyl 4-(((trifluoromethyl)sulfonyl)oxy)-2,5-dihydrofuran-2-carboxylate (600 mg, 2.07 mmol, 1.0 eq) and 2-(3-(benzyloxy)-4-methylphenyl)-4,4,5,5-tetramethyl-1,3,2-dioxaborolane (872 mg, 2.69 mmol, 1.3 eq) in THF:H₂O (5:1.6 mL) at room temperature, was added Et₃N (1.04 g, 10.35 mmol, 5.0 eq) followed by Pd(PPh₃)₄ (120 mg, 0.1 mmol, 0.05 eq) and stirred at 70° C. for 2 h. After completion of reaction by TLC, the reaction mixture was filtered through a pad of diatomaceous earth (e.g., Celite®), washed with EtOAc (15 mL), collected filtrate was concentrated under reduced pressure to afford crude product. The crude product was purified by silica gel [100-200] column chromatography [elution with 10% EtOAc in petroleum ether] to afford ethyl 4-(3-(benzyloxy)-4-methylphenyl)-2,5-dihydrofuran-2-carboxylate a yellow liquid (400 mg, Yield: 57%). TLC system: EtOAc/Hexane (20:80), R_{f} value: ˜0.5; LCMS (m/z): 339.31 (M+H)⁺;

### Synthesis of ethyl 4-(3-hydroxy-4-methylphenyl)tetrahydrofuran-2-carboxylate

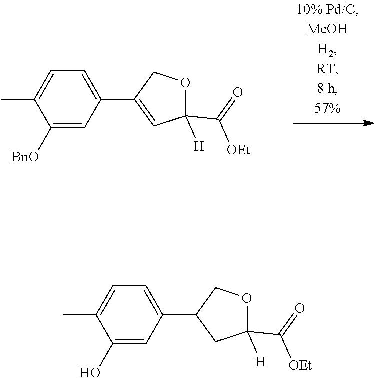 [SMILES: CCOC(=O)C1C=C(c2ccc(C)c(OCc3ccccc3)c2)CO1 ; CCOC(=O)C1CC(c2ccc(C)c(O)c2)CO1]

To a stirred solution of ethyl 4-(3-(benzyloxy)-4-methylphenyl)-2,5-dihydrofuran-2-carboxylate (700 mg, 2.07 mmol, 1.0 eq) in MeOH (10 Vol) at room temperature, was added 10% Pd/C (160 mg, 30% wt/wt) and stirred for 8 h under H₂ atmosphere. After completion of reaction by TLC, the reaction mixture was filtered through a pad of diatomaceous earth (e.g., Celite®), washed with DCM (20 mL), collected filtrate was concentrated under reduced pressure to afford crude product. The crude product was purified by silica gel [100-200] column chromatography [elution with 20% EtOAc in petroleum ether] to afford ethyl 4-(3-hydroxy-4-methylphenyl)tetrahydrofuran-2-carboxylate as a colorless gum (300 mg, Yield: 57%). TLC system: EtOAc/Hexane (30:70), R_{f} value: ˜0.3; LCMS (m/z): 251.23 (M+H)⁺.

### Synthesis of 4-(3-((5-bromo-4,6-difluoro-1-((2-(trimethylsilyl)ethoxy)methyl)-1H-benzo[d]imidazol-2-yl)oxy)-4-methylphenyl)tetrahydrofuran-2-carboxylic acid

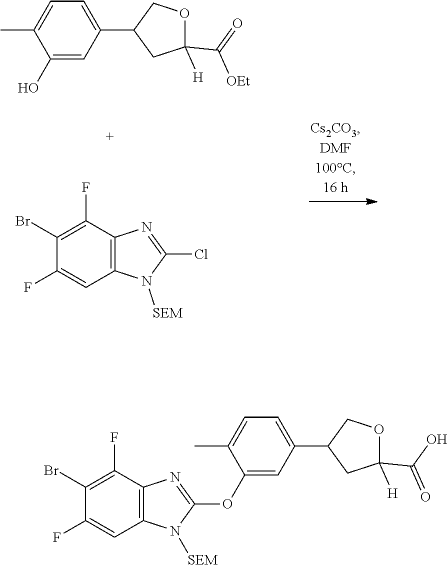 [SMILES: CCOC(=O)C1CC(c2ccc(C)c(O)c2)CO1 ; C[Si](C)(C)CCOCn1c(Cl)nc2c(F)c(Br)c(F)cc21 ; Cc1ccc(C2COC(C(=O)O)C2)cc1Oc1nc2c(F)c(Br)c(F)cc2n1COCC[Si](C)(C)C]

To a solution of ethyl 4-(3-hydroxy-4-methylphenyl)tetrahydrofuran-2-carboxylate (300 mg, 1.2 mmol, 1.0 eq) and 5-bromo-2-chloro-4,6-difluoro-1-((2-(trimethylsilyl)ethoxy)methyl)-1H-benzo[d]imidazole (505 mg, 1.56 mmol, 1.3 eq) in DMF (10 mL) at room temperature, was added Cs₂CO₃ (782 mg, 2.4 mmol, 2.0 eq) and stirred at 100° C. for 16 h. After completion of reaction by TLC, the reaction mixture was diluted with water (10 mL), acidified with 2N HCl solution (5 mL) and extracted with EtOAc (20 mL). The organic layer was dried over Na₂SO₄, filtered and concentrated under reduced pressure to afford 4-(3-((5-bromo-4,6-difluoro-1-((2-(trimethylsilyl)ethoxy)methyl)-1H-benzo[d]imidazol-2-yl)oxy)-4-methylphenyl)tetrahydrofuran-2-carboxylic acid as a yellow gum (370 mg, Crude). TLC system: EtOAc/Hexane (30:70), R_{f} value: ˜0.1; LCMS (m/z): 583.25 (M+H)⁺.

### Synthesis of ethyl 4-(3-((5-bromo-4,6-difluoro-1-((2-(trimethylsilyl)ethoxy)methyl)-1H-benzo[d]imidazol-2-yl)oxy)-4-methylphenyl)tetrahydrofuran-2-carboxylate

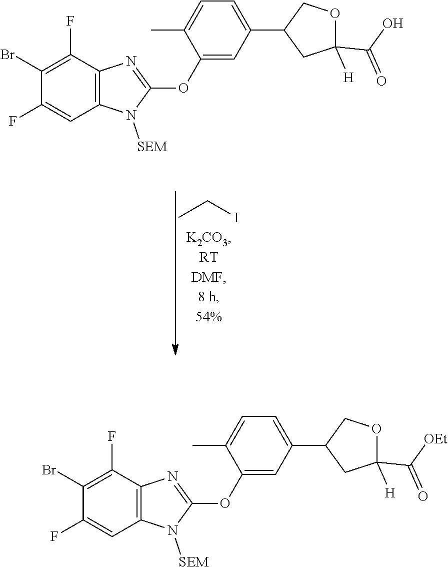

To a stirred solution of 4-(3-((5-bromo-4,6-difluoro-1-((2-(trimethylsilyl)ethoxy)methyl)-1H-benzo[d]imidazol-2-yl)oxy)-4-methylphenyl)tetrahydrofuran-2-carboxylic acid (350 mg, 0.6 mmol, 1.0 eq) in DMF (10 Vol) at 0° C., was added K₂CO₃ (250 mg, 1.8 mmol, 3.0 eq) followed by Ethyl iodide (141 mg, 0.9 mmol, 1.5 eq) and stirred at room temperature for 8 h. After completion of reaction by TLC, the reaction mixture was diluted with water (20 mL) and extracted with EtOAc (2×20 mL). The combined organic layer was dried over Na₂SO₄, filtered and concentrated under reduced pressure to afford crude product. The crude product was purified by silica gel [100-200] column chromatography [elution with 20% EtOAc in petroleum ether] to afford ethyl 4-(3-((5-bromo-4,6-difluoro-1-((2-(trimethylsilyl)ethoxy)methyl)-1H-benzo[d]imidazol-2-yl)oxy)-4-methylphenyl)tetrahydrofuran-2-carboxylate as a yellow gum (200 mg, Yield: 54%). TLC system: EtOAc/Hexane (50:50), R_{f} value: ˜0.6; LCMS (m/z): 611.39 (M+H)⁺.

### Synthesis of (4-(3-((4,6-difluoro-1-((2-(trimethylsilyl)ethoxy)methyl)-1H-benzo[d]imidazol-2-yl)oxy)-4-methylphenyl)tetrahydrofuran-2-yl)methanol

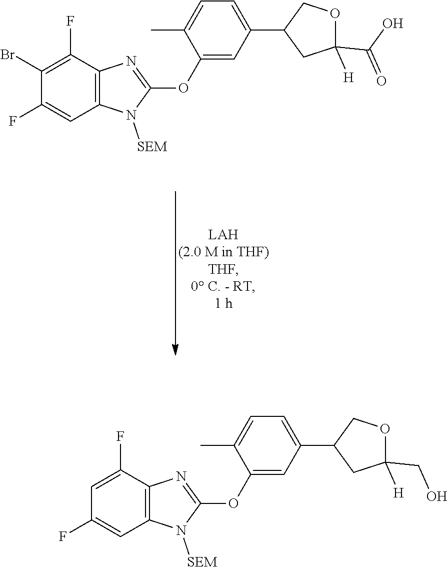

To a stirred solution of ethyl 4-(3-((5-bromo-4,6-difluoro-1-((2-(trimethylsilyl)ethoxy)methyl)-1H-benzo[d]imidazol-2-yl)oxy)-4-methylphenyl)tetrahydrofuran-2-carboxylate (250 mg, 0.41 mmol, 1.0 eq) in THF (2.5 mL) at 0° C., was added LAH (2.0 M in THF) (0.2 mL, 0.41 mmol, 1.0 eq) and stirred at room temperature for 1 h. After completion of reaction by TLC, the reaction mixture was quenched with NH₄Cl solution (15 mL) and diluted with EtOAc (10 mL). Solution was filtered through a pad of diatomaceous earth (e.g., Celite®), washed with EtOAc (15 mL). Organic layer separated and washed with H₂O (2×5 mL). The combined organic layer was dried over Na₂SO₄, filtered and concentrated under reduced pressure to afford (4-(3-((4,6-difluoro-1-((2-(trimethylsilyl)ethoxy)methyl)-1H-benzo[d]imidazol-2-yl)oxy)-4-methylphenyl)tetrahydrofuran-2-yl)methanol as a yellow gum (230 mg, Yield: Crude). TLC system: EtOAc/Hexane (100:00), R_{f} value: ˜0.5; LCMS (m/z): 491.36 (M+H)⁺.

### Synthesis of (4-(3-((5-bromo-4,6-difluoro-1-((2-(trimethylsilyl)ethoxy)methyl)-1H-benzo[d]imidazol-2-yl)oxy)-4-methylphenyl)tetrahydrofuran-2-yl)methanol

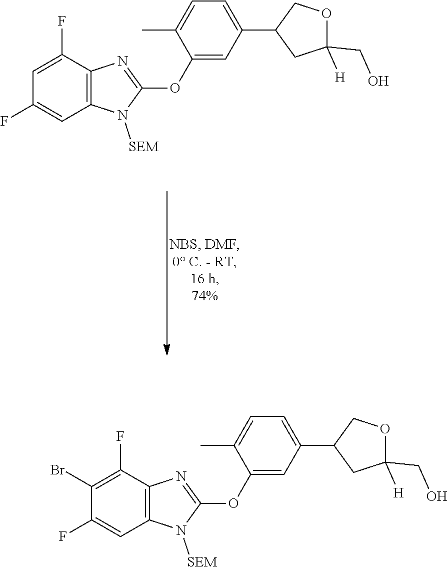

To a stirred solution of (4-(3-((4,6-difluoro-1-((2-(trimethylsilyl)ethoxy)methyl)-1H-benzo[d]imidazol-2-yl)oxy)-4-methylphenyl)tetrahydrofuran-2-yl)methanol (230 mg, 0.47 mmol, 1.0 eq) in DMF (10 Vol) at 0° C., was added NBS (125 mg, 0.7 mmol, 1.5 eq) and stirred at room temperature for 16 h. After completion of reaction by TLC, the reaction mixture was diluted with water (15 mL) and extracted with EtOAc (2×15 mL). The combined organic layer was dried over Na₂SO₄, filtered and concentrated under reduced pressure to afford crude product. The crude product was purified by silica gel [100-200] column chromatography [elution with 40% EtOAc in petroleum ether] to afford (4-(3-((5-bromo-4,6-difluoro-1-((2-(trimethylsilyl)ethoxy)methyl)-1H-benzo[d]imidazol-2-yl)oxy)-4-methylphenyl)tetrahydrofuran-2-yl)methanol as colorless gum (170 mg, Yield: 74%). TLC system: EtOAc/Hexane (100:50), R_{f} value: ˜0.5; LCMS (m/z): 603.11 (M−H)⁻; ¹H NMR (400 MHz, CDCl₃) δ: 7.22-7.19 (m, 2H), 7.10-7.05 (m, 2H), 5.52 (m, 2H), 4.21-4.19 (m, 2H), 3.83-3.79 (m, 2H), 3.67-3.60 (m, 3H), 3.52-3.48 (m, 1H), 2.27 (s, 2H), 2.39-2.37 (m, 1H), 2.35 (s, 1H), 1.90-1.87 (m, 1H), 0.98-0.94 (m, 2H), 0.02 (d, 9H).

### Synthesis of 1-(4-(4,6-difluoro-2-(5-(5-(hydroxymethyl)tetrahydrofuran-3-yl)-2-methylphenoxy)-1-((2-(trimethylsilyl)ethoxy)methyl)-1H-benzo[d]imidazol-5-yl)phenyl)cyclobutan-1-ol

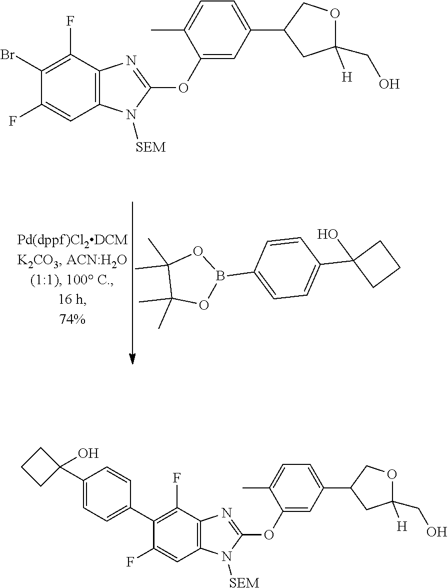 [SMILES: Cc1ccc(C2COC(CO)C2)cc1Oc1nc2c(F)c(Br)c(F)cc2n1COCC[Si](C)(C)C ; CC1(C)OB(c2ccc(C3(O)CCC3)cc2)OC1(C)C ; Cc1ccc(C2COC(CO)C2)cc1Oc1nc2c(F)c(-c3ccc(C4(O)CCC4)cc3)c(F)cc2n1COCC[Si](C)(C)C]

In a sealed tube to a degassed solution of (4-(3-((5-bromo-4,6-difluoro-1-((2-(trimethylsilyl)ethoxy)methyl)-1H-benzo[d]imidazol-2-yl)oxy)-4-methylphenyl)tetrahydrofuran-2-yl)methanol (170 mg, 0.29 mmol, 1.0 eq) and 1-(4-(4,4,5,5-tetramethyl-1,3,2-dioxaborolan-2-yl)phenyl)cyclobutan-1-ol (123 mg, 0.45 mmol, 1.5 eq) in ACN:H₂O (1:1, 1.7 mL) at room temperature, was added K₂CO₃ (124 mg, 0.9 mmol, 3.0 eq) followed by Pd(dppf)Cl₂·DCM (24 mg, 0.03 mmol, 0.1 eq) and stirred at 100° C. for 16 h. After completion of reaction by TLC, the reaction mixture was diluted with water (10 mL) and extracted with EtOAc (2×15 mL). The combined organic layer was dried over Na₂SO₄, filtered and concentrated under reduced pressure to afford crude product. The crude product was purified by silica gel [100-200] column chromatography [elution with 70% EtOAc in petroleum ether] to afford 1-(4-(4,6-difluoro-2-(5-(5-(hydroxymethyl)tetrahydrofuran-3-yl)-2-methylphenoxy)-1-((2-(trimethylsilyl)ethoxy)methyl)-1H-benzo[d]imidazol-5-yl)phenyl)cyclobutan-1-ol as a yellow gummy (140 mg, Yield: 74%). TLC system: EtOAc/Hexane (100:30), R_{f} value: ˜0.5; LCMS (m/z): 637.56 (M+H)⁺.

### Synthesis of 1-(4-(4,6-difluoro-2-(5-(5-(hydroxymethyl)tetrahydrofuran-3-yl)-2-methylphenoxy)-1H-benzo[d]imidazol-5-yl)phenyl)cyclobutan-1-ol

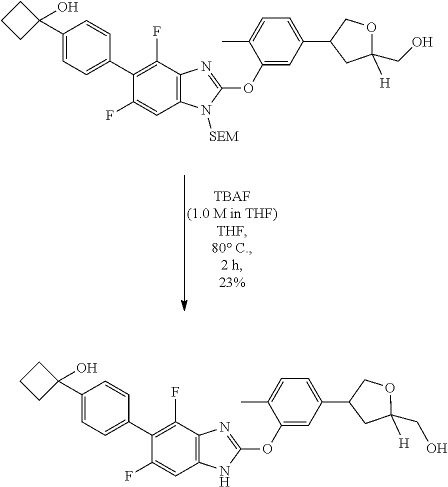 [SMILES: Cc1ccc(C2COC(CO)C2)cc1Oc1nc2c(F)c(-c3ccc(C4(O)CCC4)cc3)c(F)cc2n1COCC[Si](C)(C)C ; Cc1ccc(C2COC(CO)C2)cc1Oc1nc2c(F)c(-c3ccc(C4(O)CCC4)cc3)c(F)cc2[nH]1]

To a stirred solution of 1-(4-(4,6-difluoro-2-(5-(5-(hydroxymethyl)tetrahydrofuran-3-yl)-2-methylphenoxy)-1-((2-(trimethylsilyl)ethoxy)methyl)-1H-benzo[d]imidazol-5-yl)phenyl)cyclobutan-1-ol (150 mg, 0.23 mmol, 1.0 eq) in THF (10 Vol) at 0° C., was added TBAF (1.5 mL, 10 Vol) and stirred at 80° C. for 2 h. After completion of reaction by TLC, the reaction mixture was quenched with NH₄Cl solution (15 mL) and extracted with EtOAc (2×15 mL). The combined organic layer was dried over Na₂SO₄, filtered and concentrated under reduced pressure to afford crude product. The crude product was purified by Prep-HPLC. The collected fractions were frozen and lyophilized to afford 1-(4-(4,6-difluoro-2-(5-(5-(hydroxymethyl)tetrahydrofuran-3-yl)-2-methylphenoxy)-1H-benzo[d]imidazol-5-yl)phenyl)cyclobutan-1-ol as a white solid (28 mg, Yield: 23%). TLC system: EtOAc/Hexane (100:00), R_{f} value: ˜0.3; LCMS (m/z): 507.33 (M+H)⁺; ¹H NMR (400 MHz, DMSO-d₆) δ: 13.01 (bs, 1H), 7.59 (d, J=8.4 Hz, 2H), 7.40 (d, J=8.0 Hz, 2H), 7.29 (d, J=7.6 Hz, 1H), 7.23-7.15 (m, 3H), 5.53 (s, 1H), 4.71 (brs, 1H), 4.06-3.99 (m, 2H), 3.60 (t, J=8.4 Hz, 1H), 3.48-3.45 (m, 2H), 3.43-3.41 (m, 1H), 2.49-2.41 (m, 2H), 2.34-2.28 (m, 3H), 2.26 (s, 3H), 1.95-1.92 (m, 1H), 1.76-1.66 (m, 2H).

Prep-HPLC condition: Column X-BRIDGE (250*19) mm 5mic Buffer MP-(A): 10 mM ABC IN H2O MP-(B): ACN:WATER (80:20) Gradient (T/% B) 0/35, 15/80, 25/98 Flow Rate 15 ml/min Solubility ACN, water, THF.

### SYNTHETIC EXAMPLE 57

### Synthesis of Compound II-18

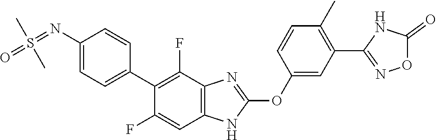 [SMILES: Cc1ccc(Oc2nc3c(F)c(-c4ccc(N=S(C)(C)=O)cc4)c(F)cc3[nH]2)cc1-c1noc(=O)[nH]1]

### Synthesis of 5-hydroxy-2-methylbenzonitrile

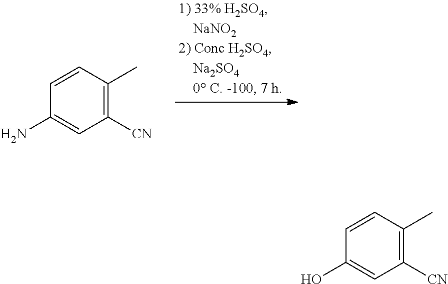

To a stirred solution of 5-amino-2-methylbenzonitrile (10 g, 75.7 mmol, 1.0 eq) in 33% H₂SO₄ (110 mL, 11 Vol), was added a solution of NaNO₂ (6.8 g, 75.7 mmol, 1.0 eq) in water (37 mL) at 0° C. and continued stirring for 2 h. In a separated flask, concentrated H₂SO₄ (80 mL, 8 Vol) was added carefully to a stirred solution of Na₂SO₄ (53.6 g, 378.2 mmol, 5.0 eq) in water (37 mL). The slurry was heated to reflux for 2 h. The prepared diazonium salt was added dropwise to the refluxing mixture and continued stirred for 5 hours. After completion of reaction by TLC, the reaction mixture was extracted with EtOAc (2×300 mL). The combined organic layer was dried over Na₂SO₄, filtered and concentrated under reduced pressure. Obtained crude was purified by silica column (100-200 mesh) chromatography to afford 5-hydroxy-2-methylbenzonitrile as a yellow solid (2.6 mg, Yield: 26%). TLC system: EtOAc/Hexane (30:70%), R_{f} value: ˜0.2; LCMS (m/z): 132.02 (M−H)⁻.

### Synthesis of (Z)-N′,5-dihydroxy-2-methylbenzimidamide

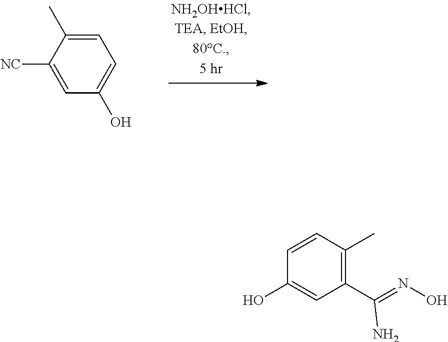

To a stirred solution of 5-hydroxy-2-methylbenzonitrile (2.6 g, 19.5 mmol, 1.0 eq) in EtOH (91 mL, 35 Vol) at room temperature, was added TEA (8.1 mL, 58.6 mmol, 3.0 eq), NH₂OH·HCl (2.7 g, 39.1 mmol, 2.0 eq) and stirred at 80° C. for 5 h. After completion of reaction by TLC, the reaction mixture evaporated to under reduced pressure, diluted with water and extracted with EtOAc (2×250 mL). The combined organic layer was dried over Na₂SO₄, filtered and concentrated under reduced pressure to afford (Z)-N′,5-dihydroxy-2-methylbenzimidamide as a colorless gummy solid (2.6 g, crude). TLC system: EtOAc (100%), R_{f} value: ˜0.1; LCMS (m/z): 167.14 (M+H)⁺.

### Synthesis of ethyl (Z)-((((ethoxycarbonyl)oxy) imino) (5-hydroxy-2-methylphenyl)methyl)carbamate

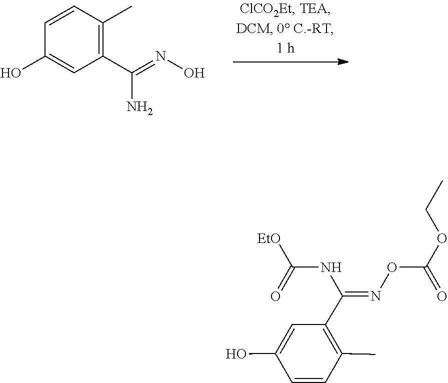 [SMILES: Cc1ccc(O)cc1/C(N)=N/O ; CCOC(=O)N/C(=N\OC(=O)OCC)c1cc(O)ccc1C]

To a stirred solution of (Z)-N′,5-dihydroxy-2-methylbenzimidamide (2.6 g, 15.66 mmol, 1.0 eq) in DCM (26 mL, 10 Vol) at 0° C., was added TEA (4.3 mL, 31.3 mmol, 2 eq), Ethyl chloroformate (1.78 mL, 18.79 mmol, 1.2 eq), and stirred at RT for 1 h. After completion of reaction by TLC, the reaction mixture was quenched with ice cold water and extracted with EtOAc (2×100 mL). The combined organic layer was dried over Na₂SO₄ and concentrated under reduced pressure. Obtained crude was purified by silica column (100-200 mesh) to afford ethyl (Z)-((((ethoxycarbonyl)oxy) imino) (5-hydroxy-2-methylphenyl)methyl)carbamate as colorless gummy liquid (3.4 g, Yield: 56% in 2 steps).TLC system: EtOAc/Hexane (20:80), R_{f} value: ˜0.3; LCMS (m/z): 311.21 (M+H)⁺.

### Synthesis of 3-(5-hydroxy-2-methylphenyl)-1,2,4-oxadiazol-5(4H)-one

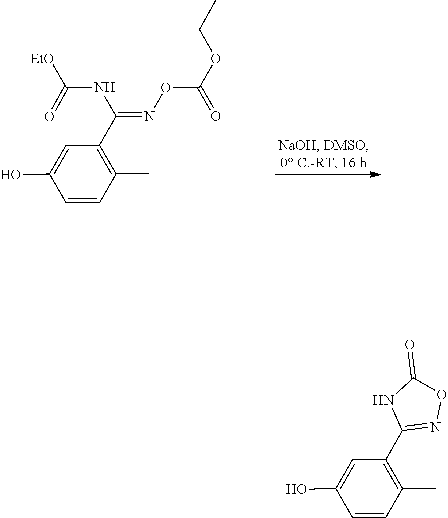 [SMILES: CCOC(=O)N/C(=N\OC(=O)OCC)c1cc(O)ccc1C ; Cc1ccc(O)cc1-c1noc(=O)[nH]1]

To a stirred solution of ethyl (Z)-((((ethoxycarbonyl)oxy) imino) (5-hydroxy-2-methylphenyl)methyl)carbamate (3.4 g, 10.96 mmol, 1.0 eq) in DMSO (34 mL, 10 Vol) at 0° C., was added NaOH (570.1 mg, 14.25 mmol, 1.3 eq), and stirred at RT for 16 h. After completion of reaction by TLC, the reaction mixture was acidified with 1N aq. HCl (200 mL) and extracted with EtOAc (2×200 mL). The combined organic layer was dried over Na₂SO₄, filtered and concentrated under reduced pressure. Obtained crude was purified by silica column (100-200 mesh) to afford to 3-(5-hydroxy-2-methylphenyl)-1,2,4-oxadiazol-5(4H)-one as a colorless gummy liquid (1.2 g, Yield: 57%). TLC system: EtOAc (100%), R_{f} value: ˜0.2; LCMS (m/z): 193.05 (M+H)⁺.

### Synthesis of 3-(5-((6-bromo-5,7-difluoro-1-((2-(trimethylsilyl)ethoxy)methyl)-1H-benzo[d]imidazol-2-yl)oxy)-2-methylphenyl)-1,2,4-oxadiazol-5(4H)-one

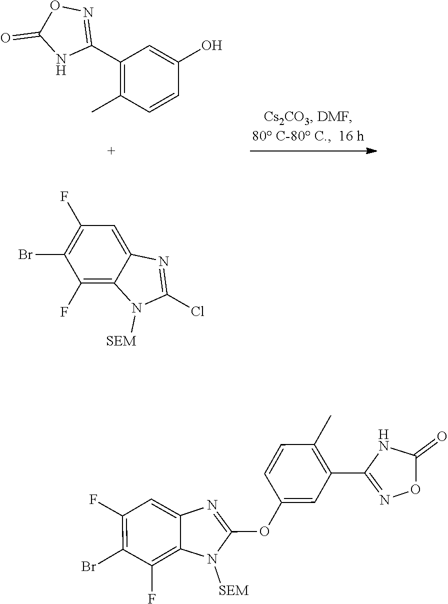 [SMILES: Cc1ccc(O)cc1-c1noc(=O)[nH]1 ; C[Si](C)(C)CCOCn1c(Cl)nc2cc(F)c(Br)c(F)c21 ; Cc1ccc(Oc2nc3cc(F)c(Br)c(F)c3n2COCC[Si](C)(C)C)cc1-c1noc(=O)[nH]1]

To a stirred solution of 6-bromo-2-chloro-5,7-difluoro-1-((2-(trimethylsilyl)ethoxy)methyl)-1H-benzo[d]imidazole (800 mg, 2.02 mmol, 1.0 eq) and 3-(5-hydroxy-2-methylphenyl)-1,2,4-oxadiazol-5(4H)-one (504.3 mg, 2.62 mmol, 1.3 eq) in DMF (8 mL, 10 Vol) at room temperature, was added Cs₂CO₃ (1.32 g, 4.04 mmol, 2.0 eq) and stirred at 80° C. for 16 h. After completion of reaction by TLC, reaction mixture was diluted with water (100 mL) and extracted with EtOAc (2×150 mL). The combined organic layer was dried over Na₂SO₄, filtered and concentrated under reduced pressure to afford 3-(5-((6-bromo-5,7-difluoro-1-((2-(trimethylsilyl)ethoxy)methyl)-1H-benzo[d]imidazol-2-yl)oxy)-2-methylphenyl)-1,2,4-oxadiazol-5(4H)-one as a colorless solid (900 mg, Yield: 81%). TLC system: EtOAc/Hexane (50:50), R_{f} value: ˜0.2; LCMS (m/z): 553.02 (M+H)⁺.

### Synthesis of 3-(5-((5-(4-((dimethyl(oxo)-16-sulfaneylidene)amino)phenyl)-4,6-difluoro-1-((2-(trimethylsilyl)ethoxy)methyl)-1H-benzo[d]imidazol-2-yl)oxy)-2-methylphenyl)-1,2,4-oxadiazol-5(4H)-one

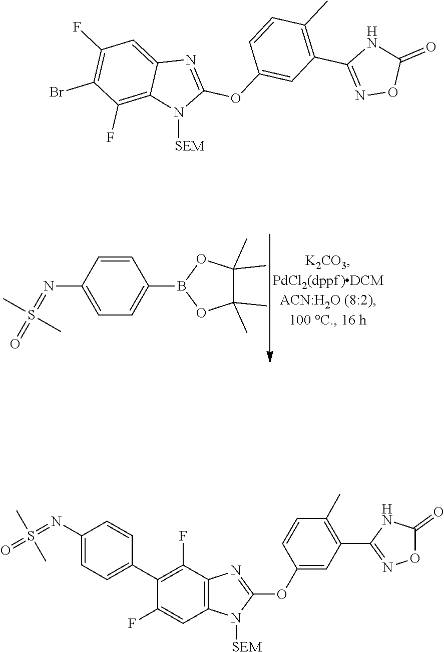

In a sealed tube to a degassed solution of 3-(5-((6-bromo-5,7-difluoro-1-((2-(trimethylsilyl)ethoxy)methyl)-1H-benzo[d]imidazol-2-yl)oxy)-2-methylphenyl)-1,2,4-oxadiazol-5(4H)-one (300 mg, 0.54 mmol, 1.0 eq) and dimethyl ((4-(4,4,5,5-tetramethyl-1,3,2-dioxaborolan-2-yl)phenyl) imino)-16-sulfanone (401 mg, 1.35 mmol, 2.5 eq) in ACN:H₂O (8:2) (6 mL, 20 Vol) at room temperature, was added K₂CO₃ (223.5 mg, 1.62 mmol, 3.0 eq) followed by PdCl₂(dppf)·DCM (44.06 mg, 0.054 mmol, 0.1 eq) and stirred at 100° C. for 16 h. After completion of reaction by TLC, the reaction mixture was diluted with water (20 mL) and extracted with EtOAc (2×100 mL). The combined organic layer was dried over Na₂SO₄, filtered and concentrated under reduced pressure. Obtained crude was purified by reverse phase column and pure fraction were evaporated to afford 3-(5-((5-(4-((dimethyl(oxo)-16-sulfaneylidene)amino)phenyl)-4,6-difluoro-1-((2-(trimethylsilyl)ethoxy)methyl)-1H-benzo[d]imidazol-2-yl)oxy)-2-methylphenyl)-1,2,4-oxadiazol-5(4H)-one as a colorless solid (250 mg, Yield: 72%). TLC system: EtOAc (100), R_{f} value: ˜0.1; LCMS (m/z): 642.42 (M+H)⁺.

### Synthesis of 3-(5-((5-(4-((dimethyl(oxo)-16-sulfaneylidene)amino)phenyl)-4,6-difluoro-1H-benzo[d]imidazol-2-yl)oxy)-2-methylphenyl)-1,2,4-oxadiazol-5(4H)-one

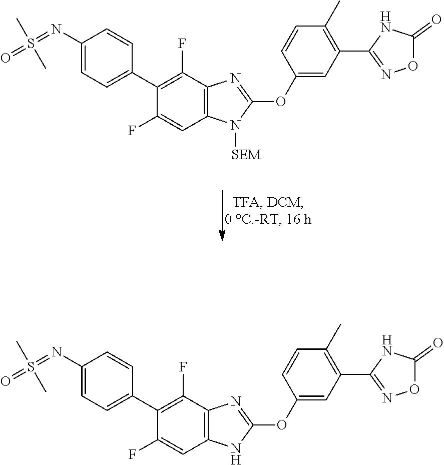 [SMILES: Cc1ccc(Oc2nc3c(F)c(-c4ccc(N=S(C)(C)=O)cc4)c(F)cc3n2COCC[Si](C)(C)C)cc1-c1noc(=O)[nH]1 ; Cc1ccc(Oc2nc3c(F)c(-c4ccc(N=S(C)(C)=O)cc4)c(F)cc3[nH]2)cc1-c1noc(=O)[nH]1]

To a stirred solution of 3-(5-((5-(4-((dimethyl(oxo)-16-sulfaneylidene)amino)phenyl)-4,6-difluoro-1-((2-(trimethylsilyl)ethoxy)methyl)-1H-benzo[d]imidazol-2-yl)oxy)-2-methylphenyl)-1,2,4-oxadiazol-5(4H)-one (250 mg, 0.39 mmol, 1.0 eq) in DCM (5 mL, 20 Vol) at 0° C., was added TFA (2.5 mL, 10 Vol) and stirred at room temperature for 16 h. After completion of reaction by TLC, the reaction mixture concentrated, washed with pentane (20 mL) and purified by Prep-HPLC. Collected fractions were lyophilized to afford 3-(5-((5-(4-((dimethyl(oxo)-16-sulfaneylidene)amino)phenyl)-4,6-difluoro-1H-benzo[d]imidazol-2-yl)oxy)-2-methylphenyl)-1,2,4-oxadiazol-5(4H)-one as an off white solid (61 mg, Yield: 26%). TLC system: EtOAc (100%), R_{f} value: ˜0.1; LCMS (m/z): 512.30 (M+H)⁺; ¹H NMR (400 MHz, DMSO-d₆) δ: 13.21-12.75 (m, 2H), 7.64-7.63 (m, 1H), 7.61-7.58 (m, 1H), 7.54-7.52 (m, 1H), 7.26-7.20 (m, 3H), 7.02 (d, J=8.4 Hz, 2H), 3.26 (s, 6H), 2.49 (s, 3H).

Prep-HPLC condition: YMC C-18 (250*20.0) mm 5mic, Buffer MP-(A): 10 mM ABC in Water MP-(B): ACN:Water (80:20); Gradient (T/% B): 0/25, 15/60, 20/90, 25/98; Flow Rate: 13 ml/min; Solubility: Water/ACN/THF

### SYNTHETIC EXAMPLE 58

### Synthesis of Compound II-19

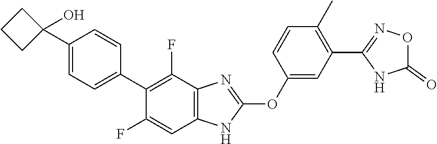 [SMILES: Cc1ccc(Oc2nc3c(F)c(-c4ccc(C5(O)CCC5)cc4)c(F)cc3[nH]2)cc1-c1noc(=O)[nH]1]

### Synthesis of 3-(5-((4,6-difluoro-5-(4-(1-hydroxycyclobutyl)phenyl)-1-((2-(trimethylsilyl)ethoxy)methyl)-1H-benzo[d]imidazol-2-yl)oxy)-2-methylphenyl)-1,2,4-oxadiazol-5(4H)-one

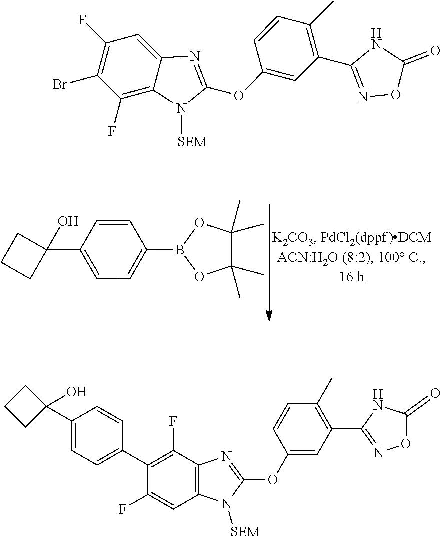 [SMILES: Cc1ccc(Oc2nc3cc(F)c(Br)c(F)c3n2COCC[Si](C)(C)C)cc1-c1noc(=O)[nH]1 ; CC1(C)OB(c2ccc(C3(O)CCC3)cc2)OC1(C)C ; Cc1ccc(Oc2nc3c(F)c(-c4ccc(C5(O)CCC5)cc4)c(F)cc3n2COCC[Si](C)(C)C)cc1-c1noc(=O)[nH]1]

In a sealed tube to a degassed solution of 3-(5-((6-bromo-5,7-difluoro-1-((2-(trimethylsilyl)ethoxy)methyl)-1H-benzo[d]imidazol-2-yl)oxy)-2-methylphenyl)-1,2,4-oxadiazol-5(4H)-one (300 mg, 0.54 mmol, 1.0 eq) and 1-(4-(4,4,5,5-tetramethyl-1,3,2-dioxaborolan-2-yl)phenyl)cyclobutan-1-ol (223.3 mg, 0.81 mmol, 1.5 eq) in ACN:H₂O (8:2) (6 mL, 20 Vol) at room temperature, was added K₂CO₃ (224.97 mg, 1.63 mmol, 3.0 eq) followed by PdCl₂(dppf)·DCM (44.3 mg, 0.054 mmol, 0.1 eq) and stirred at 100° C. for 16 h. After completion of reaction by TLC, the reaction mixture was diluted with water (20 mL) and extracted with EtOAc (2×100 mL). The combined organic layer was dried over Na₂SO₄, filtered and concentrated under reduced pressure. Obtained crude was purified by reverse phase C₁₈ column chromatography [eluted with ˜39% ACN and 0.1% FA in H₂O] and pure fraction were evaporated to afford 3-(5-((4,6-difluoro-5-(4-(1-hydroxycyclobutyl)phenyl)-1-((2-(trimethylsilyl)ethoxy)methyl)-1H-benzo[d]imidazol-2-yl)oxy)-2-methylphenyl)-1,2,4-oxadiazol-5(4H)-one as a colorless solid (300 mg, Yield: 89%). TLC system: EtOAc (100), R_{f} value: ˜0.2; LCMS (m/z): 621.48 (M+H)⁺.

### Synthesis of 3-(5-((4,6-difluoro-5-(4-(1-hydroxycyclobutyl)phenyl)-1H-benzo[d]imidazol-2-yl)oxy)-2-methylphenyl)-1,2,4-oxadiazol-5(4H)-one

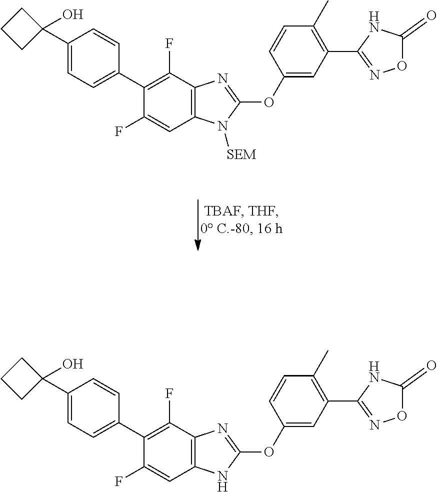 [SMILES: Cc1ccc(Oc2nc3c(F)c(-c4ccc(C5(O)CCC5)cc4)c(F)cc3n2COCC[Si](C)(C)C)cc1-c1noc(=O)[nH]1 ; Cc1ccc(Oc2nc3c(F)c(-c4ccc(C5(O)CCC5)cc4)c(F)cc3[nH]2)cc1-c1noc(=O)[nH]1]

To a stirred solution of 3-(5-((4,6-difluoro-5-(4-(1-hydroxycyclobutyl)phenyl)-1-((2-(trimethylsilyl)ethoxy)methyl)-1H-benzo[d]imidazol-2-yl)oxy)-2-methylphenyl)-1,2,4-oxadiazol-5(4H)-one (300 mg, 0.48 mmol, 1.0 eq) in THF (3 mL, 10 Vol) at 0° C., was added TBAF (3 mL, 10 Vol) and stirred at 80° C. for 16 h. After completion of reaction by TLC, the reaction mixture concentrated under reduced pressure and washed with pentane (20 mL) to afford crude compound. Purified by Prep-HPLC, collected fractions were lyophilized to afford 3-(5-((4,6-difluoro-5-(4-(1-hydroxycyclobutyl)phenyl)-1H-benzo[d]imidazol-2-yl)oxy)-2-methylphenyl)-1,2,4-oxadiazol-5(4H)-one as an off white solid (42 mg, Yield: 17%). TLC system: EtOAc/Hexane (70:30), R_{f} value: ˜0.1; LCMS (m/z): 491.33 (M+H)⁺; ¹H NMR (400 MHz, DMSO-d₆) δ: 13.05-12.80 (m, 2H), 7.64-7.58 (m, 4H), 7.54-7.52 (m, 1H), 7.41-7.39 (m, 2H), 7.24 (d, J=9.6 Hz, 1H), 5.55 (s, 1H), 2.49 (s, 3H), 2.47-2.41 (m, 2H), 2.33-2.26 (m, 2H), 1.96-1.91 (m, 1H), 1.72-1.64 (m, 1H).

Prep-HPLC condition: YMC TRIART C-18 (250*20.0) mm 5mic, Buffer MP-(A): 10 mM ABC in Water MP-(B): ACN:Water (80:20); Gradient (T/% B): 0/40, 14/58, 15/58, 20/90, 25/98; Flow Rate: 13 ml/min; Solubility: Water/ACN.

### SYNTHETIC EXAMPLE 59

### Synthesis of Compound I-74

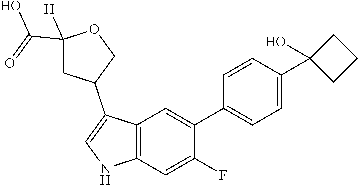 [SMILES: O=C(O)C1CC(c2c[nH]c3cc(F)c(-c4ccc(C5(O)CCC5)cc4)cc23)CO1]

### Synthesis of 4-(6-fluoro-5-(4-(1-hydroxycyclobutyl)phenyl)-1H-indol-3-yl)tetrahydrofuran-2-carboxylic acid

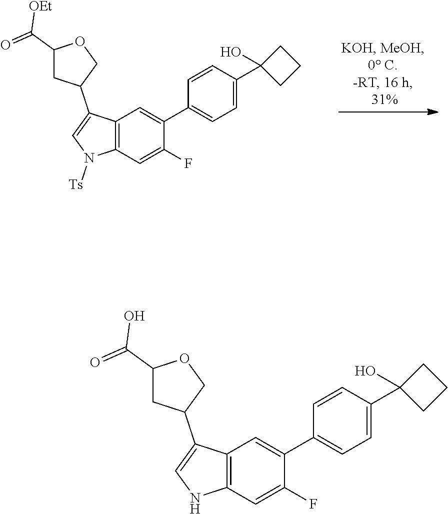 [SMILES: CCOC(=O)C1CC(c2cn(S(=O)(=O)c3ccc(C)cc3)c3cc(F)c(-c4ccc(C5(O)CCC5)cc4)cc23)CO1 ; O=C(O)C1CC(c2c[nH]c3cc(F)c(-c4ccc(C5(O)CCC5)cc4)cc23)CO1]

To a stirred solution of ethyl 4-(6-fluoro-5-(4-(1-hydroxycyclobutyl)phenyl)-1- to syl-1H-indol-3-yl)tetrahydrofuran-2-carboxylate (130 mg, 0.22 mmol, 1.0 eq) in MeOH (10 Vol) at 0° C., was added KOH (63 mg, 1.13 mmol, 5.0 eq) and stirred at room temperature for 16 h. After completion of reaction by TLC, the reaction mixture was concentrated under reduced pressure to afford crude product. The crude product was purified by Prep-HPLC, collected fractions were lyophilized to afford 1-(4-(6-fluoro-3-(5-(hydroxymethyl)tetrahydrofuran-3-yl)-1H-indol-5-yl)phenyl)cyclobutan-1-ol as a white solid (28 mg, Yield: 31%). TLC system: MeOH/DCM (10:90), R_{f} value: ˜0.1; LCMS (m/z): 394.23 (M−H)⁻; ¹H NMR (400 MHz, DMSO-d₆) δ: 12.9 (bs, 1H), 11.01 (s, 1H), 7.65 (d, J=7.6 Hz, 1H), 7.57-7.51 (m, 4H), 7.24-7.21 (m, 2H), 5.51 (s, 1H), 4.48 (t, J=8.0 Hz, 1H), 4.24 (t, J=7.6 Hz, 1H), 3.82-378 (m, 1H), 3.68-3.64 (m, 1H), 2.76-2.67 (m, 1H), 2.49-2.40 (m, 2H), 2.39-2.27 (m, 2H), 2.09-2.06 (m, 1H), 1.95-1.92 (m, 1H), 1.67-1.66 (m, 1H).

Prep-HPLC condition: Column GEMINI NX C18 (250*21.2) mm 5mic Buffer MP-(A): 0.1% FA in WATER MP-(B): ACN:WATER (80:20) Gradient (T/% B) 0/40, 15/75, 20/98 Flow Rate 13 ml/min Solubility ACN, water, THF.

### SYNTHETIC EXAMPLE 60

### Synthesis of Compound II-20

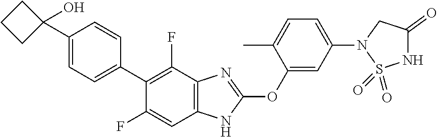 [SMILES: Cc1ccc(N2CC(=O)NS2(=O)=O)cc1Oc1nc2c(F)c(-c3ccc(C4(O)CCC4)cc3)c(F)cc2[nH]1]

### Synthesis of ethyl (3-(benzyloxy)-4-methylphenyl)glycinate

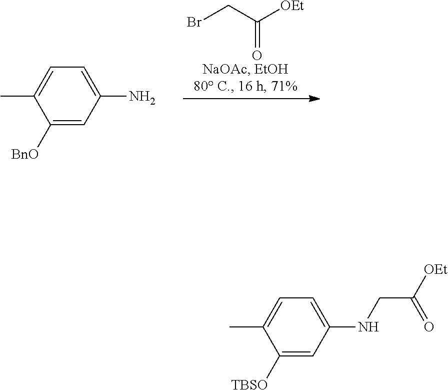 [SMILES: CCOC(=O)CBr ; Cc1ccc(N)cc1OCc1ccccc1 ; CCOC(=O)CNc1ccc(C)c(O[Si](C)(C)C(C)(C)C)c1]

To a stirred solution of 3-(benzyloxy)-4-methylaniline (2.0 g, 9.39 mmol, 1.0 eq) in EtOH (10 Vol) at room temperature, was added NaOAc (1.54 g, 9.39 mmol, 1.0 eq) followed by ethyl 2-bromoacetate (1.57 g, 18.78 mmol, 2.0 eq) and stirred at 80° C. for 8 h. After completion of reaction by TLC, the reaction mixture was diluted with water (30 mL) and extracted with EtOAc (2× 30 mL). The combined organic layer was dried over Na₂SO₄, filtered and concentrated under reduced pressure to afford crude product. The crude product was purified by silica gel [60-120 mesh] column chromatography [elution with 10% EtOAc in petroleum ether] to afford ethyl (3-(benzyloxy)-4-methylphenyl)glycinate as a brown gum (2.0 g, Yield: 71%). TLC system: EtOAc/Hexane (20:80), R_{f} value: ˜0.5; LCMS (m/z): 300.33 (M+H)⁺; ¹H NMR (400 MHz, CDCl₃) δ: 7.45-7.31 (m, 5H), 6.98-6.96 (m, 1H), 6.24-6.23 (m, 1H), 6.19-6.17 (m, 1H), 5.04 (s, 2H), 4.34-4.16 (m, 2H), 2.17 (s, 3H), 1.29 (t, J=7.2 Hz, 3H).

### Synthesis of benzyl (chlorosulfonyl)carbamate

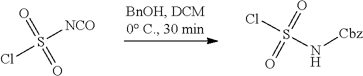

To a stirred solution of sulfurisocyanatidic chloride (5.0 g, 3.55 mmol, 1.0 eq) in DCM (25 Vol) at 0° C., was added solution of BnOH (4.02 g, 3.72 mmol, 1.0 eq) in DCM (10 Vol) dropwise over 30 min. After completion of addition, reaction mixture was concentrated under reduced pressure to afford benzyl (chlorosulfonyl)carbamate as a brown solid (6.0 g, Yield: Crude). Crude was directly used for next step. LCMS (m/z): 249.3 (M+H)⁺.

### Synthesis of ethyl N-(3-(benzyloxy)-4-methylphenyl)-N-(N-((benzyloxy)carbonyl)sulfamoyl)glycinate

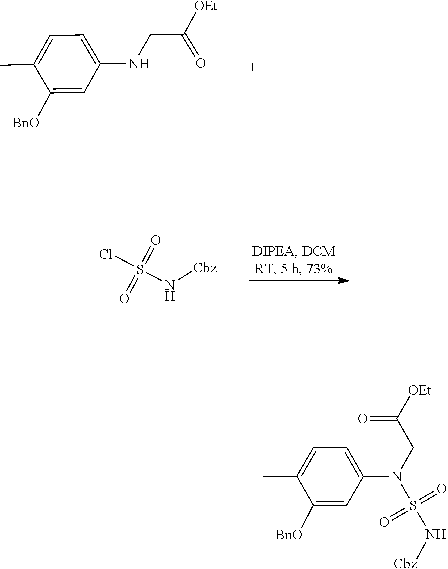 [SMILES: CCOC(=O)CNc1ccc(C)c(OCc2ccccc2)c1 ; O=C(NS(=O)(=O)Cl)OCc1ccccc1 ; CCOC(=O)CN(c1ccc(C)c(OCc2ccccc2)c1)S(=O)(=O)NC(=O)OCc1ccccc1]

To a stirred solution of ethyl (3-(benzyloxy)-4-methylphenyl)glycinate (2.0 g, 6.66 mmol, 1.0 eq) in DCM (10 Vol) at room temperature, was added DIPEA (2.57 g, 19.98 mmol, 3.0 eq) followed by benzyl (chlorosulfonyl)carbamate (1.84 g, 7.99 mmol, 1.2 eq) and stirred for 5 h. After completion of reaction by TLC, the reaction mixture was diluted with water (30 mL) and extracted with DCM (2×30 mL). The combined organic layer was dried over Na₂SO₄, filtered and concentrated under reduced pressure to afford crude product. The crude product was purified by silica gel [100-200 mesh] column chromatography [elution with 40% EtOAc in petroleum ether] to afford ethyl N-(3-(benzyloxy)-4-methylphenyl)-N-(N-((benzyloxy)carbonyl)sulfamoyl)glycinate as a brown gum (2.5 g, Yield: 73%). TLC system: EtOAc/Hexane (50:50), R_{f} value: ˜0.2; LCMS (m/z): 511.16 (M−H)⁻; ¹H NMR (400 MHz, CDCl₃) δ: 7.45-7.40 (m, 1H), 7.39-7.30 (m, 9H), 7.07-7.04 (m, 2H), 6.77-6.75 (m, 1H), 5.21 (s, 2H), 4.99 (s, 2H), 4.53 (s, 2H), 4.17 (q, J=6.8 Hz, 2H), 2.24 (s, 3H), 1.29-1.23 (m, 3H).

### Synthesis of ethyl N-(3-hydroxy-4-methylphenyl)-N-sulfamoylglycinate

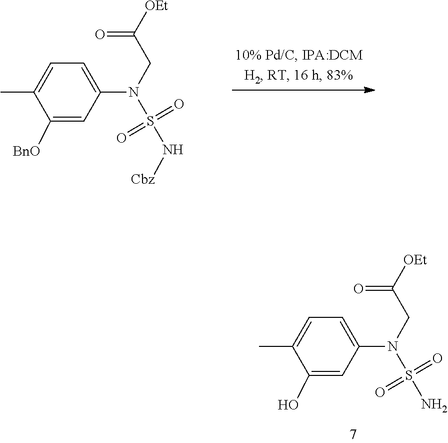 [SMILES: CCOC(=O)CN(c1ccc(C)c(OCc2ccccc2)c1)S(=O)(=O)NC(=O)OCc1ccccc1 ; CCOC(=O)CN(c1ccc(C)c(O)c1)S(N)(=O)=O]

To a stirred solution of ethyl N-(3-(benzyloxy)-4-methylphenyl)-N-(N-((benzyloxy)carbonyl)sulfamoyl)glycinate (1.5 g, 2.93 mmol, 1.0 eq) in IPA: DCM (5:1, 10 Vol) at room temperature, was added 10% Pd/C (300 mg, 20% wt/wt) and stirred at room temperature for 16 h under H₂ atmosphere. After completion of reaction by TLC, the reaction mixture was filtered through a pad of diatomaceous earth (e.g., Celite®), washed with DCM (30 mL) and collected filtrate was concentrated under reduced pressure to afford crude product. The crude product was purified by silica gel [60-120 mesh] column chromatography [elution with 50% EtOAc in petroleum ether] to afford ethyl N-(3-hydroxy-4-methylphenyl)-N-sulfamoylglycinate as a brown gum (700 mg, Yield: 83%). TLC system: EtOAc/Hexane (100:00), R_{f} value: ˜0.6; LCMS (m/z): 287.14 (M−H)⁺.

### Synthesis of 5-(3-hydroxy-4-methylphenyl)-1,2,5-thiadiazolidin-3-one 1,1-dioxide

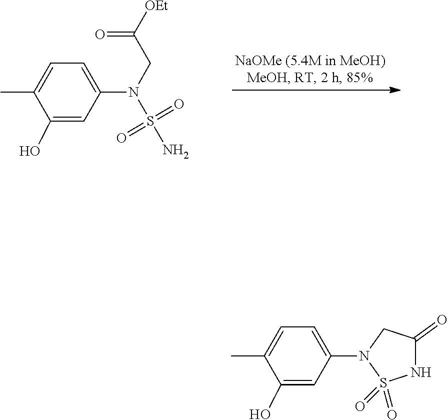 [SMILES: CCOC(=O)CN(c1ccc(C)c(O)c1)S(N)(=O)=O ; Cc1ccc(N2CC(=O)NS2(=O)=O)cc1O]

To a stirred solution of ethyl N-(3-hydroxy-4-methylphenyl)-N-sulfamoylglycinate (700 mg, 2.43 mmol, 1.0 eq) in MeOH (10 Vol) at room temperature, was added NaOMe (5.4 M in MeOH) (1 Vol) and stirred for 2 h. After completion of reaction by TLC, the reaction mixture was concentrated under reduced pressure and purified by reverse phase column chromatography [elution with 40% ACN in 0.1% FA in H₂O] to afford 5-(3-hydroxy-4-methylphenyl)-1,2,5-thiadiazolidin-3-one 1,1-dioxide as brown solid (500 mg, Yield: 85%). TLC system: MeOH/DCM (10:90), R_{f} value: ˜0.1 (Not lifted); LCMS (m/z): 241.10 (M−H)⁻; ¹H NMR (400 MHz, DMSO-d₆) δ: 8.52 (s, 1H), 6.83 (d, J=8.0 Hz, 1H), 6.55 (bs, 1H), 6.21 (bs, 1H), 3.82 (s, 2H), 1.99 (s, 3H).

### Synthesis of 5-(3-((5-bromo-4,6-difluoro-1-((2-(trimethylsilyl)ethoxy)methyl)-1H-benzo[d]imidazol-2-yl)oxy)-4-methylphenyl)-1,2,5-thiadiazolidin-3-one 1,1-dioxide

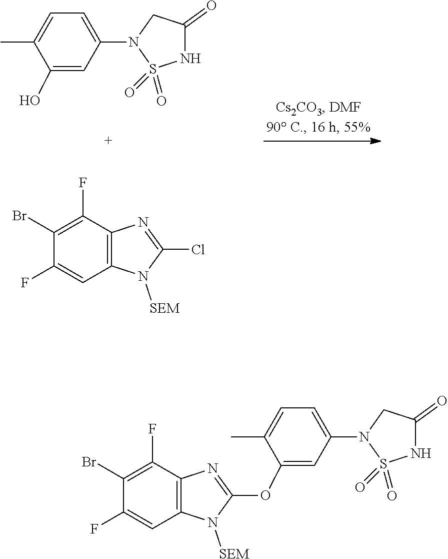 [SMILES: Cc1ccc(N2CC(=O)NS2(=O)=O)cc1O ; C[Si](C)(C)CCOCn1c(Cl)nc2c(F)c(Br)c(F)cc21 ; Cc1ccc(N2CC(=O)NS2(=O)=O)cc1Oc1nc2c(F)c(Br)c(F)cc2n1COCC[Si](C)(C)C]

To a stirred solution of 5-(3-hydroxy-4-methylphenyl)-1,2,5-thiadiazolidin-3-one 1,1-dioxide (500 mg, 2.10 mmol, 1.2 eq) in DMF (10 Vol) at room temperature, was added Cs₂CO₃ (1.73 g, 5.31 mmol, 3.0 eq) followed by 5-bromo-2-chloro-4,6-difluoro-1-((2-(trimethylsilyl)ethoxy)methyl)-1H-benzo[d]imidazole (700 mg, 1.77 mmol, 1.0 eq) and stirred at 90° C. for 16 h. After completion of reaction by TLC, the reaction mixture was filtered through a pad of diatomaceous earth (e.g., Celite®), washed with EtOAc (50 mL) and collected filtrate was concentrated under reduced pressure to afford crude product. The crude product was purified by reverse phase column chromatography [elution with 60% ACN in 0.1% FA in H₂O] to afford 5-(3-((5-bromo-4,6-difluoro-1-((2-(trimethylsilyl)ethoxy)methyl)-1H-benzo[d]imidazol-2-yl)oxy)-4-methylphenyl)-1,2,5-thiadiazolidin-3-one 1,1-dioxide as a brown solid (550 mg, Yield: 55%). TLC system: MeOH/DCM (10:90), R_{f} value: ˜0.2; LCMS (m/z): 602.9 (M+H)⁺.

### Synthesis of 5-(3-((4,6-difluoro-5-(4-(1-hydroxycyclobutyl)phenyl)-1-((2-(trimethylsilyl)ethoxy)methyl)-1H-benzo[d]imidazol-2-yl)oxy)-4-methylphenyl)-1,2,5-thiadiazolidin-3-one 1,1-dioxide

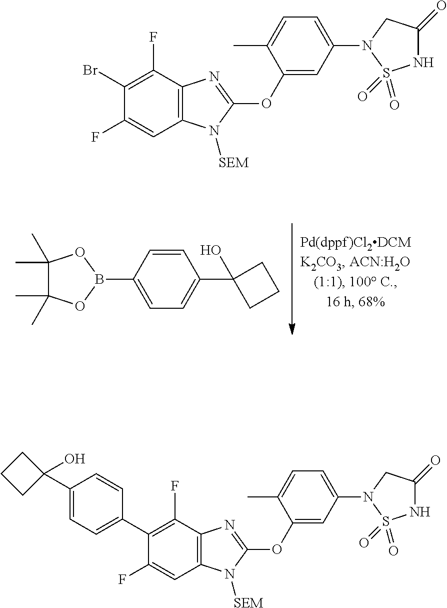 [SMILES: Cc1ccc(N2CC(=O)NS2(=O)=O)cc1Oc1nc2c(F)c(Br)c(F)cc2n1COCC[Si](C)(C)C ; CC1(C)OB(c2ccc(C3(O)CCC3)cc2)OC1(C)C ; Cc1ccc(N2CC(=O)NS2(=O)=O)cc1Oc1nc2c(F)c(-c3ccc(C4(O)CCC4)cc3)c(F)cc2n1COCC[Si](C)(C)C]

In a sealed tube, to a degassed solution of 5-(3-((5-bromo-4,6-difluoro-1-((2-(trimethylsilyl)ethoxy)methyl)-1H-benzo[d]imidazol-2-yl)oxy)-4-methylphenyl)-1,2,5-thiadiazolidin-3-one 1,1-dioxide (400 mg, 0.66 mmol, 1.0 eq) and 1-(4-(4,4,5,5-tetramethyl-1,3,2-dioxaborolan-2-yl)phenyl)cyclobutan-1-ol (218 mg, 0.79 mmol, 1.5 eq) in ACN:H₂O (1:1, 10 Vol) at room temperature, was added K₂CO₃ (273 mg, 1.98 mmol, 3.0 eq) followed by Pd(dppf)Cl₂·DCM (54 mg, 0.066 mmol, 0.1 eq) and stirred at 100° C. for 16 h. After completion of reaction by TLC, the reaction mixture was concentrated under reduced pressure and purified by reverse phase column chromatography [elution with 45% ACN in 0.1% FA in H₂O] to afford 5-(3-((4,6-difluoro-5-(4-(1-hydroxycyclobutyl)phenyl)-1-((2-(trimethylsilyl)ethoxy)methyl)-1H-benzo[d]imidazol-2-yl)oxy)-4-methylphenyl)-1,2,5-thiadiazolidin-3-one 1,1-dioxide as a brown solid (300 mg, Yield: 68%). TLC system: MeOH/DCM (20:80), R_{f} value: ˜0.3; LCMS (m/z): 671.3 (M+H)⁺; regio-isomers with respect to SEM protection on imidazole.

### Synthesis of 5-(3-((4,6-difluoro-5-(4-(1-hydroxycyclobutyl)phenyl)-1H-benzo[d]imidazol-2-yl)oxy)-4-methylphenyl)-1,2,5-thiadiazolidin-3-one 1,1-dioxide

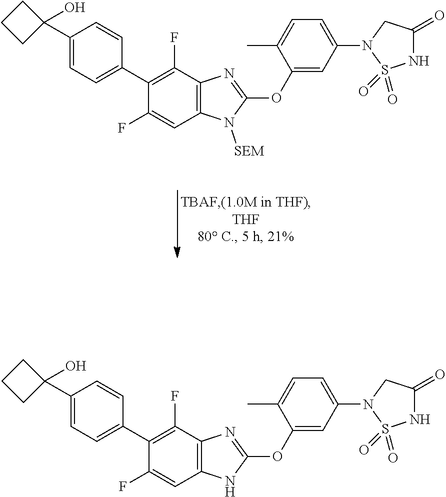 [SMILES: Cc1ccc(N2CC(=O)NS2(=O)=O)cc1Oc1nc2c(F)c(-c3ccc(C4(O)CCC4)cc3)c(F)cc2n1COCC[Si](C)(C)C ; Cc1ccc(N2CC(=O)NS2(=O)=O)cc1Oc1nc2c(F)c(-c3ccc(C4(O)CCC4)cc3)c(F)cc2[nH]1]

To a stirred solution of 5-(3-((4,6-difluoro-5-(4-(1-hydroxycyclobutyl)phenyl)-1-((2-(trimethylsilyl)ethoxy)methyl)-1H-benzo[d]imidazol-2-yl)oxy)-4-methylphenyl)-1,2,5-thiadiazolidin-3-one 1,1-dioxide (300 mg, 0.45 mmol, 1.0 eq) in THF (10 Vol) at 0° C., was added TBAF (1.0 M in THF) (10 Vol) and stirred at 80° C. for 5 h. After completion of reaction by TLC, the reaction mixture was quenched with NH₄Cl solution (20 mL) and extracted with DCM (2×20 mL). The combined organic layer was dried over Na₂SO₄, filtered and concentrated under reduced pressure to afford crude product. The crude product was purified by reverse phase column chromatography [elution with 32% ACN in 0.1% FA in H₂O] to afford desired product. Re-purified by Prep-HPLC. The collected fractions were lyophilized to afford desired product with TBAF impurities. Re-purified by reverse phase column chromatography [elution with 28% ACN in 0.1% FA in H₂O] to afford 5-(3-((4,6-difluoro-5-(4-(1-hydroxycyclobutyl)phenyl)-1H-benzo[d]imidazol-2-yl)oxy)-4-methylphenyl)-1,2,5-thiadiazolidin-3-one 1,1-dioxide as white solid (50 mg, Yield: 21%). TLC system: MeOH/DCM (20:80), R_{f} value: ˜0.1; LCMS (m/z): 541.24 (M+H)⁺; ¹H NMR (400 MHz, DMSO-d₆) δ: 7.58 (d, J=8.4 Hz, 2H), 7.40 (d, J=8.0 Hz, 2H), 7.26 (d, J=8.4 Hz, 1H), 7.19 (d, J=9.2 Hz, 1H), 7.01-7.00 (m, 1H), 6.98 (dd, J=2.0 Hz, 8.0 Hz, 1H), 4.03 (s, 2H), 2.49-2.41 (m, 2H), 2.33-2.25 (m, 2H), 2.08 (s, 3H), 1.98-1.88 (m, 1H), 1.75-1.72 (m, 1H).

Prep-HPLC condition: Column YMC C-18 (250*21.2 mm) 5μ; Buffer MP-(A): 10 Mm ABC in H2O, MP-(B): ACN:WATER (80:20); Gradient (T/% B) 0/30, 10/65, 25/98; Flow Rate: 15 ml/min; Solubility ACN, WATER, THF

### SYNTHETIC EXAMPLE 61

### Synthesis of Compound II-21

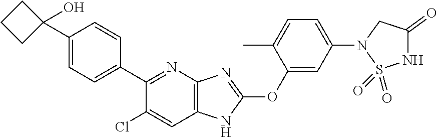 [SMILES: Cc1ccc(N2CC(=O)NS2(=O)=O)cc1Oc1nc2nc(-c3ccc(C4(O)CCC4)cc3)c(Cl)cc2[nH]1]

### Synthesis of 5,6-dichloro-3-nitropyridin-2-amine

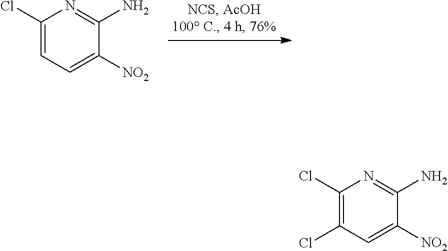 [SMILES: Nc1nc(Cl)ccc1[N+](=O)[O-] ; Nc1nc(Cl)c(Cl)cc1[N+](=O)[O-]]

To a stirred solution of 6-chloro-3-nitropyridin-2-amine (20 g, 115.60 mmol, 1.0 eq) in acetic acid (5 Vol) at room temperature, was added NCS (18.45 g, 138.73 mmol, 1.2 eq) and stirred at 100° C. for 4 h. After completion of reaction by TLC, the reaction mixture was cooled to room temperature, solid filtered, washed with EtOH (60 mL) and dried under reduced pressure to afford 5,6-dichloro-3-nitropyridin-2-amine as yellow solid (18 g, Yield: 76%). TLC system: EtOAc/Hexane (20:80), R_{f} value: ˜0.5; LCMS (m/z): 208.00 (M+H)⁺.

### Synthesis of 5-chloro-6-iodo-3-nitropyridin-2-amine

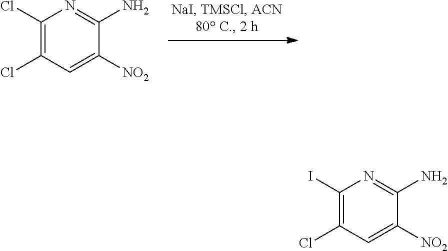 [SMILES: Nc1nc(Cl)c(Cl)cc1[N+](=O)[O-] ; Nc1nc(I)c(Cl)cc1[N+](=O)[O-]]

To a stirred solution of 5,6-dichloro-3-nitropyridin-2-amine (11 g, 53.14 mmol, 1.0 eq) in ACN (10 Vol) at room temperature, was added TMSCl (5.79 g, 53.14 mmol, 1.0 eq) followed by NaI (23.9 g, 159.4 mmol, 3.0 eq) and stirred at 80° C. for 2 h. After completion of reaction by TLC, the reaction mixture was diluted with water (70 mL) and extracted with EtOAc (2×70 mL). The organic layer was dried over Na₂SO₄, filtered and concentrated under reduced pressure to afford 5-chloro-6-iodo-3-nitropyridin-2-amine as a yellow solid (17 g, Yield: Crude). TLC system: EtOAc/Hexane (20:80), R_{f} value: ˜0.6; LCMS (m/z): 300.04 (M+H)⁺.

### Synthesis of 5-chloro-6-iodopyridine-2,3-diamine

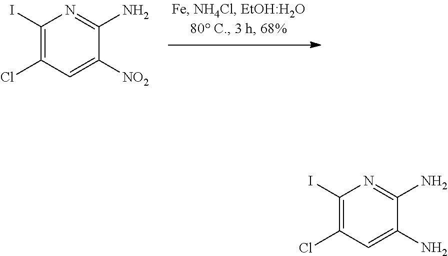 [SMILES: Nc1nc(I)c(Cl)cc1[N+](=O)[O-] ; Nc1cc(Cl)c(I)nc1N]

To a stirred solution of 5-chloro-6-iodo-3-nitropyridin-2-amine (17 g, 56.85 mmol, 1.0 eq) in EtOH:H₂O (1:1, 10 Vol) at room temperature, was added NH₄Cl (12.14 g, 227.4 mmol, 4.0 eq), Fe (12.69 g, 227.4 mmol, 4.0 eq) and stirred at 80° C. for 3 h. After completion of reaction by TLC, the reaction mixture was filtered through a pad of diatomaceous earth (e.g., Celite®), washed with EtOAc (50 mL) and collected filtrate was washed with water (70 mL). The organic layer was dried over Na₂SO₄, filtered and concentrated under reduced pressure to afford crude product. The crude product was purified by silica gel [60-120] column chromatography [elution with 40% EtOAc in petroleum ether] to afford 5-chloro-6-iodopyridine-2,3-diamine as brown solid (10.5 g, Yield: 68%). TLC system: EtOAc/Hexane (50:50), R_{f} value: ˜0.3. Crude directly used for next step.

### Synthesis of 6-chloro-5-iodo-1,3-dihydro-2H-imidazo[4,5-b]pyridine-2-thione

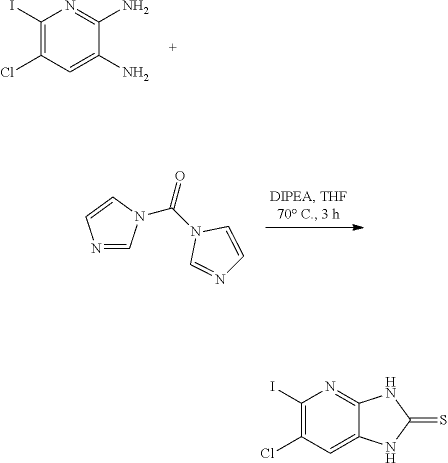 [SMILES: Nc1cc(Cl)c(I)nc1N ; O=C(n1ccnc1)n1ccnc1 ; S=c1[nH]c2cc(Cl)c(I)nc2[nH]1]

To a stirred solution of 5-chloro-6-iodopyridine-2,3-diamine (10.5 g, 39.03 mmol, 1.0 eq) in THF (10 Vol) at room temperature, was added DIPEA (16 mL, 117.1 mmol, 3.0 eq) followed by Thio-CDI (10.4 g, 58.54 mmol, 1.5 eq) and stirred at 70° C. for 3 h. After completion of reaction by TLC, the reaction mixture was quenched with 2N HCl solution (70 mL) and extracted with EtOAc (2×70 mL). The combined organic layer was dried over Na₂SO₄, filtered and concentrated under reduced pressure to afford 6-chloro-5-iodo-1,3-dihydro-2H-imidazo[4,5-b]pyridine-2-thione as yellow solid (12 g, Yield: Crude). TLC system: EtOAc/Hexane (50:50), R_{f} value: ˜0.4; LCMS (m/z): 311.96 (M+H)⁺.

### Synthesis of 6-chloro-5-iodo-2-(methylthio)-1H-imidazo[4,5-b]pyridine

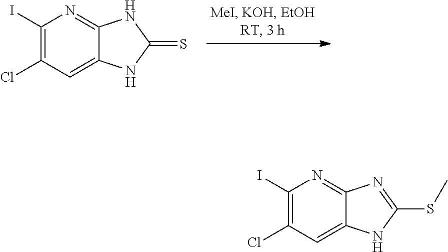

To a stirred solution of 6-chloro-5-iodo-1,3-dihydro-2H-imidazo[4,5-b]pyridine-2-thione (12 g, 38.58 mmol, 1.0 eq) in EtOH (20 Vol) at room temperature, was added KOH (2.6 g, 46.30 mmol, 1.2 eq) followed by MeI (2.4 mL, 38.58 mmol, 1.0 eq) and stirred at room temperature for 3 h. After completion of reaction by TLC, the reaction mixture was quenched with 2N HCl solution (50 mL), diluted with water (100 mL) and extracted with EtOAc (2×100 mL). The combined organic layer was dried over Na₂SO₄, filtered and concentrated under reduced pressure to afford 6-chloro-5-iodo-2-(methylthio)-1H-imidazo[4,5-b]pyridine as a yellow solid (13.3 g, Yield: Crude). TLC system: EtOAc/Hexane (30:70), R_{f} value: ˜0.4; LCMS (m/z): 326.04 (M+H)⁺; ¹H NMR (400 MHz, DMSO-d₆) δ: 13.35 (bs, 1H), 8.07 (s, 1H), 2.70 (s, 3H).

### Synthesis of 6-chloro-5-iodo-2-(methylsulfonyl)-1H-imidazo[4,5-b]pyridine

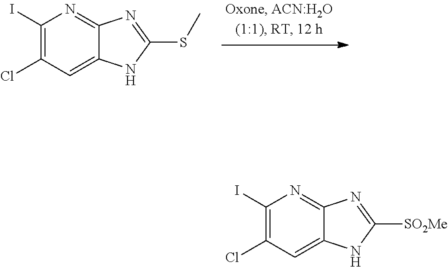

To a stirred solution of 6-chloro-5-iodo-2-(methylthio)-1H-imidazo[4,5-b]pyridine (13.3 g, 40.92 mmol, 1.0 eq) in ACN:H₂O (1:1, 10 Vol) at room temperature, was added Oxone (27.6 g, 90.02 mmol, 2.2 eq) and stirred at room temperature for 12 h. After completion of reaction by TLC, the reaction mixture was diluted with water (70 mL) and extracted with EtOAc (2×70 mL). The combined organic layer was dried over Na₂SO₄, filtered and concentrated under reduced pressure to afford 6-chloro-5-iodo-2-(methylsulfonyl)-1H-imidazo[4,5-b]pyridine as a yellow solid (12.5 g, Yield: Crude). TLC system: EtOAc/Hexane (50:50), R_{f} value: ˜0.4; LCMS (m/z): 358.00 (M+H)⁺.

### Synthesis of 6-chloro-5-iodo-2-(methylsulfonyl)-1-((2-(trimethylsilyl)ethoxy)methyl)-1H-imidazo[4,5-b]pyridine

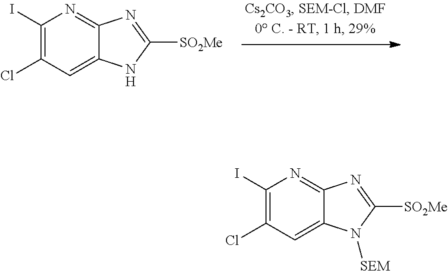 [SMILES: CS(=O)(=O)c1nc2nc(I)c(Cl)cc2[nH]1 ; C[Si](C)(C)CCOCn1c(S(C)(=O)=O)nc2nc(I)c(Cl)cc21]

To a stirred solution of 6-chloro-5-iodo-2-(methylsulfonyl)-1H-imidazo[4,5-b]pyridine (2 g, 5.60 mmol, 1.0 eq) in DMF (10 Vol) at 0° C., was added Cs₂CO₃ (3.65 g, 11.20 mmol, 2.0 eq) and stirred for 30 min. Then SEM-Cl (1.4 g, 8.4 mmol, 1.5 eq) was added to reaction mixture and stirred at room temperature for 1 h. After completion of reaction by TLC, the reaction mixture was quenched with water (20 mL) and extracted with EtOAc (2×20 mL). The combined organic layer was dried over Na₂SO₄, filtered and concentrated under reduced pressure to afford crude product. The crude product was purified by reverse phase column chromatography [elution with 80% ACN in 0.1% FA in H₂O] to afford 6-chloro-5-iodo-2-(methylsulfonyl)-1-((2-(trimethylsilyl)ethoxy)methyl)-1H-imidazo[4,5-b]pyridine as a yellow gum (800 mg, Yield: 29%). TLC system: EtOAc/Hexane (30:70), R_{f} value: ˜0.6; ¹H NMR (400 MHz, CdCl₃) δ: 8.18 (s, 1H), 5.99 (s, 2H), 3.72-3.68 (m, 2H), 3.47 (s, 3H), 1.00-0.96 (m, 2H), 0.01 (s, 9H).

### Synthesis of 5-(3-((6-chloro-5-iodo-1-((2-(trimethylsilyl)ethoxy)methyl)-1H-imidazo[4,5-b]pyridin-2-yl)oxy)-4-methylphenyl)-1,2,5-thiadiazolidin-3-one 1,1-dioxide

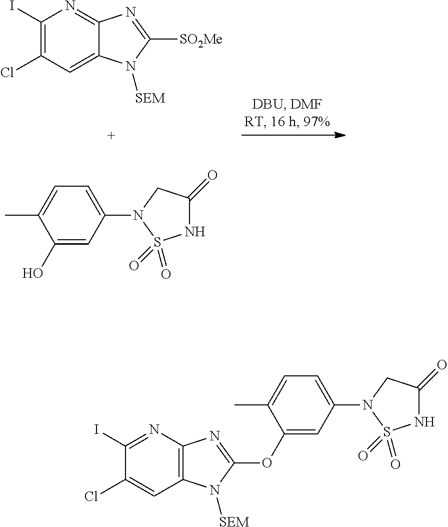 [SMILES: C[Si](C)(C)CCOCn1c(S(C)(=O)=O)nc2nc(I)c(Cl)cc21 ; Cc1ccc(N2CC(=O)NS2(=O)=O)cc1O ; Cc1ccc(N2CC(=O)NS2(=O)=O)cc1Oc1nc2nc(I)c(Cl)cc2n1COCC[Si](C)(C)C]

To a stirred solution of 6-chloro-5-iodo-2-(methylsulfonyl)-1-((2-(trimethylsilyl)ethoxy)methyl)-1H-imidazo[4,5-b]pyridine (500 mg, 1.02 mmol, 1.0 eq) in DMF (10 Vol) at room temperature, was added DBU (468 mg, 3.08 mmol, 3.0 eq) followed by 5-(3-hydroxy-4-methylphenyl)-1,2,5-thiadiazolidin-3-one 1,1-dioxide (373 mg, 1.54 mmol, 1.5 eq) and stirred at room temperature for 16 h. After completion of reaction by TLC, the reaction mixture was diluted with water (20 mL) and extracted with EtOAc (2×20 mL). The combined organic layer was concentrated under reduced pressure to afford crude product. The crude product was purified by silica gel [100-200] column chromatography [elution with 7% MeOH in DCM] to afford 5-(3-((6-chloro-5-iodo-1-((2-(trimethylsilyl)ethoxy)methyl)-1H-imidazo[4,5-b]pyridin-2-yl)oxy)-4-methylphenyl)-1,2,5-thiadiazolidin-3-one 1,1-dioxide) as yellow gum (650 mg, Yield: 97%). TLC system: MeOH/DCM (10:90), R_{f} value: ˜0.5; LCMS (m/z): 650.25 (M+H)⁺.

### Synthesis of 5-(3-((6-chloro-5-(4-(1-hydroxycyclobutyl)phenyl)-1-((2-(trimethylsilyl)ethoxy)methyl)-1H-imidazo[4,5-b]pyridin-2-yl)oxy)-4-methylphenyl)-1,2,5-thiadiazolidin-3-one 1,1-dioxide

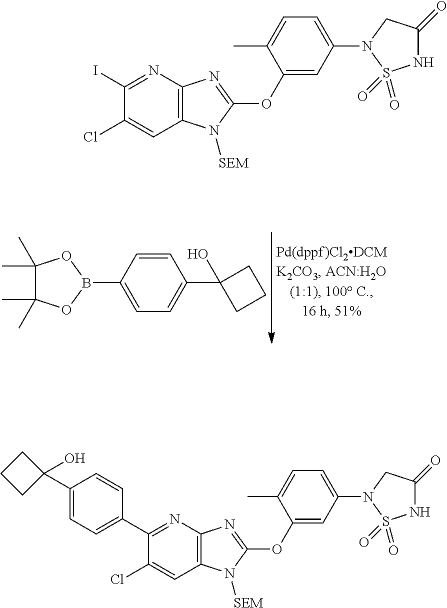 [SMILES: Cc1ccc(N2CC(=O)NS2(=O)=O)cc1Oc1nc2nc(I)c(Cl)cc2n1COCC[Si](C)(C)C ; CC1(C)OB(c2ccc(C3(O)CCC3)cc2)OC1(C)C ; Cc1ccc(N2CC(=O)NS2(=O)=O)cc1Oc1nc2nc(-c3ccc(C4(O)CCC4)cc3)c(Cl)cc2n1COCC[Si](C)(C)C]

In a sealed tube to a degassed solution of 5-(3-((6-chloro-5-iodo-1-((2-(trimethylsilyl)ethoxy)methyl)-1H-imidazo[4,5-b]pyridin-2-yl)oxy)-4-methylphenyl)-1,2,5-thiadiazolidin-3-one 1,1-dioxide (380 mg, 0.58 mmol, 1.0 eq) and 1-(4-(4,4,5,5-tetramethyl-1,3,2-dioxaborolan-2-yl)phenyl)cyclobutan-1-ol (192 mg, 0.7 mmol, 1.2 eq) in ACN:H₂O (1:1, 10 Vol) at room temperature, was added K₂CO₃ (240 mg, 1.74 mmol, 3.0 eq) followed by Pd(dppf)Cl₂·DCM (47 mg, 0.058 mmol, 0.1 eq) and stirred at 100° C. for 16 h. After completion of reaction by TLC, the reaction mixture was diluted with water (20 mL) and extracted with EtOAc (2×20 mL). The combined organic layer was concentrated under reduced pressure to afford crude product. The crude product was purified by reverse phase column chromatography [elution with 70% ACN in 0.1% FA in H₂O] to afford 5-(3-((6-chloro-5-(4-(1-hydroxycyclobutyl)phenyl)-1-((2-(trimethylsilyl)ethoxy)methyl)-1H-imidazo[4,5-b]pyridin-2-yl)oxy)-4-methylphenyl)-1,2,5-thiadiazolidin-3-one 1,1-dioxide as yellow solid (200 mg, Yield: 51%). TLC system: MeOH/DCM (10:90), R_{f} value: ˜0.3; LCMS (m/z): 670.3 (M+H)⁺. ¹H NMR (400 MHz, CDCl₃) δ: 7.86 (s, 1H), 7.74 (d, J=8.0 Hz, 2H), 7.60 (d, J=6.0 Hz, 3H), 7.22-7.14 (m, 2H), 5.68 (m, 2H), 4.39 (s, 2H), 3.49 (s, 2H), 2.63-2.61 (m, 3H), 2.17 (s, 3H), 2.09-2.04 (m, 2H), 1.76-1.74 (m, 2H), 1.00 (t, J=8.0 Hz, 2H), 0.08 (s, 9H).

### Synthesis of 5-(3-((6-chloro-5-(4-(1-hydroxycyclobutyl)phenyl)-1H-imidazo[4,5-b]pyridin-2-yl)oxy)-4-methylphenyl)-1,2,5-thiadiazolidin-3-one 1,1-dioxide

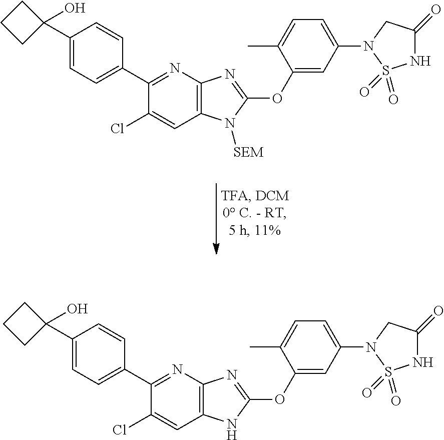 [SMILES: Cc1ccc(N2CC(=O)NS2(=O)=O)cc1Oc1nc2nc(-c3ccc(C4(O)CCC4)cc3)c(Cl)cc2n1COCC[Si](C)(C)C ; Cc1ccc(N2CC(=O)NS2(=O)=O)cc1Oc1nc2nc(-c3ccc(C4(O)CCC4)cc3)c(Cl)cc2[nH]1]

To a stirred solution of 5-(3-((6-chloro-5-(4-(1-hydroxycyclobutyl)phenyl)-1-((2-(trimethylsilyl)ethoxy)methyl)-1H-imidazo[4,5-b]pyridin-2-yl)oxy)-4-methylphenyl)-1,2,5-thiadiazolidin-3-one 1,1-dioxide (160 mg, 0.24 mmol, 1.0 eq) in DCM (10 Vol) at 0° C., was added TFA (1.6 mL, 10 Vol) and stirred at room temperature for 5 h. After completion of reaction by TLC, the reaction mixture was concentrated under reduced pressure and purified by Prep-HPLC. The collected fractions were frozen and lyophilized to afford desired product which was re-purified by reverse phase column chromatography [elution with 50% ACN in 0.1% FA in H₂O] to afford 5-(3-((6-chloro-5-(4-(1-hydroxycyclobutyl)phenyl)-1H-imidazo[4,5-b]pyridin-2-yl)oxy)-4-methylphenyl)-1,2,5-thiadiazolidin-3-one 1,1-dioxide as a white solid (14 mg, Yield: 11%). TLC system: MeOH/DCM (20:80), R_{f} value: ˜0.3; LCMS (m/z): 540.21 (M+H)⁺; ¹H NMR (400 MHz, DMSO-d₆) δ: 13.27 (bs, 1H), 7.94 (s, 1H), 7.63-7.56 (m, 4H), 7.25 (d, J=8.4 Hz, 1H), 7.01-6.97 (m, 2H), 5.55 (bs, 1H), 3.97 (s, 2H), 2.49-2.41 (m, 2H), 2.34-2.26 (m, 2H), 2.10 (s, 3H), 1.97-1.91 (m, 1H), 1.78-1.76 (m, 1H).

Prep-HPLC condition: Column X-Bridge C-18 (250*19 mm) 5u Buffer MP-(A): 10 mM ABC in H2O MP-(B): ACN Gradient (T/% B) 0/20, 15/70, 20/95 Flow Rate 15 ml/min Solubility ACN:WATER:THF

### SYNTHETIC EXAMPLE 62

### Synthesis of Compound II-24

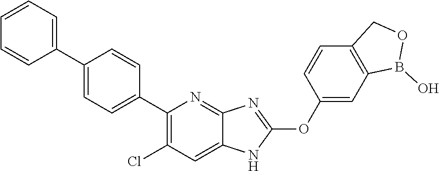 [SMILES: OB1OCc2ccc(Oc3nc4nc(-c5ccc(-c6ccccc6)cc5)c(Cl)cc4[nH]3)cc21]

### Synthesis of 5,6-dichloro-3-nitropyridin-2-amine

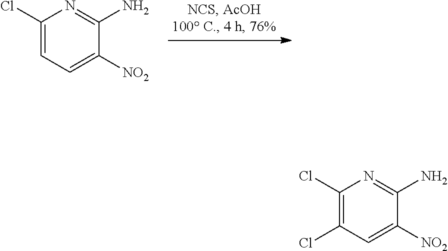 [SMILES: Nc1nc(Cl)ccc1[N+](=O)[O-] ; Nc1nc(Cl)c(Cl)cc1[N+](=O)[O-]]

To a stirred solution of 6-chloro-3-nitropyridin-2-amine (20 g, 115.6 mmol, 1.0 eq) in acetic acid (5 Vol) at room temperature, was added NCS (18.45 g, 138.7 mmol, 1.2 eq) and stirred at 100° C. for 4 h. After completion of reaction by TLC, the reaction mixture was cooled to room temperature, precipitated solid was filtered, washed with EtOH (60 mL) and dried under vacuum to afford 5,6-dichloro-3-nitropyridin-2-amine as yellow solid (18 g, Yield: 76%). TLC system: EtOAc/Hexane (20:80), R_{f} value: ˜0.5; LCMS (m/z): 208.00 (M+H)⁺.

### Synthesis of 6-([1,1′-biphenyl]-4-yl)-5-chloro-3-nitropyridin-2-amine

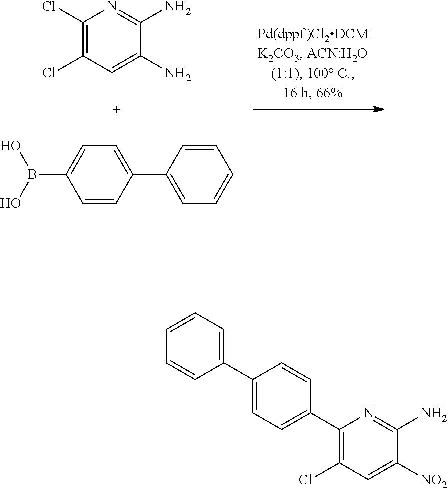 [SMILES: Nc1cc(Cl)c(Cl)nc1N ; OB(O)c1ccc(-c2ccccc2)cc1 ; Nc1nc(-c2ccc(-c3ccccc3)cc2)c(Cl)cc1[N+](=O)[O-]]

In a sealed tube, to a degassed solution of 5,6-dichloro-3-nitropyridin-2-amine (3 g, 14.49 mmol, 1.0 eq) and [1,1′-biphenyl]-4-ylboronic acid (3.44 g, 17.39 mmol, 1.2 eq) in ACN:H₂O (1:1, 10 Vol) at room temperature, was added K₂CO₃ (5.99 g, 43.47 mmol, 3.0 eq) followed by Pd(dppf)Cl₂·DCM (1.18 g, 1.45 mmol, 0.1 eq) and stirred at 100° C. for 16 h. After completion of reaction by TLC, the reaction mixture was diluted with water (50 mL) and extracted with EtOAc (2×50 mL). The combined organic layer was dried over Na₂SO₄, filtered and concentrated under reduced pressure. The crude product was purified by silica gel [100-200] column chromatography [elution with 25% EtOAc in petroleum ether] to afford 6-([1,1′-biphenyl]-4-yl)-5-chloro-3-nitropyridin-2-amine as yellow solid (3.1 g, Yield: 66%). TLC system: EtOAc/Hexane (20:80), R_{f} value: ˜0.3; LCMS (m/z): 326.20 (M+H)⁺; ¹H NMR (400 MHz, CDCl₃) δ: 8.55 (s, 1H), 8.12 (brs, 2H), 7.82 (brs, 4H), 7.76 (d, J=7.2 Hz, 2H), 7.51 (t, J=7.2 Hz, 2H), 7.44-7.40 (m, 1H). Confirmed by nOe.

### Synthesis of 6-([1,1′-biphenyl]-4-yl)-5-chloropyridine-2,3-diamine

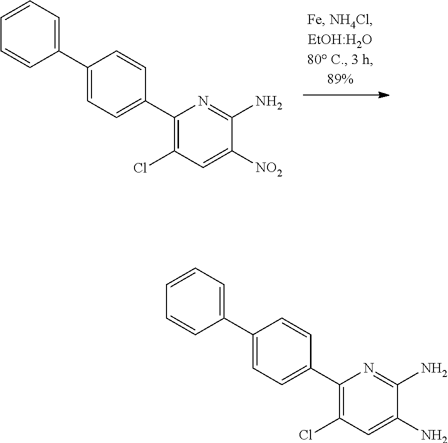 [SMILES: Nc1nc(-c2ccc(-c3ccccc3)cc2)c(Cl)cc1[N+](=O)[O-] ; Nc1cc(Cl)c(-c2ccc(-c3ccccc3)cc2)nc1N]

To a stirred solution of 6-([1,1′-biphenyl]-4-yl)-5-chloro-3-nitropyridin-2-amine (3.1 g, 9.54 mmol, 1.0 eq) in EtOH:H₂O (1:1, 10 Vol) at room temperature, was added NH₄Cl (2.06 g, 38.15 mmol, 4.0 eq), Fe (2.13 g, 38.15 mmol, 4.0 eq) and stirred at 80° C. for 3 h. After completion of reaction by TLC, the reaction mixture was filtered through a pad of diatomaceous earth (e.g., Celite®), washed with EtOAc (30 mL) and collected filtrate was washed with water (20 mL). Organic layer was dried over Na₂SO₄, filtered and concentrated under reduced pressure to afford crude product. The crude product was purified by silica gel [60-120] column chromatography [elution with 40% EtOAc in petroleum ether] to afford 6-([1,1′-biphenyl]-4-yl)-5-chloropyridine-2,3-diamine as brown solid (2.5 g, Yield: 89%). TLC system: EtOAc/Hexane (50:50), R_{f} value: ˜0.3; LCMS (m/z): 296.23 (M+H)⁺; ¹H NMR (400 MHz, CDCl₃) δ: 7.75 (d, J=8.4 Hz, 2H), 7.66-7.62 (m, 4H), 7.44 (t, J=7.6 Hz, 2H), 7.35-7.33 (m, 1H), 7.02 (s, 1H), 4.39 (br, 2H), 3.45 (br, 2H).

### Synthesis of 5-([1,1′-biphenyl]-4-yl)-6-chloro-1,3-dihydro-2H-imidazo[4,5-b]pyridine-2-thione

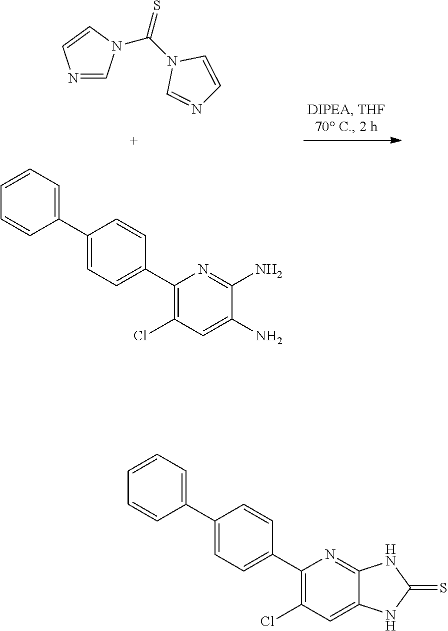 [SMILES: S=C(n1ccnc1)n1ccnc1 ; Nc1cc(Cl)c(-c2ccc(-c3ccccc3)cc2)nc1N ; S=c1[nH]c2cc(Cl)c(-c3ccc(-c4ccccc4)cc3)nc2[nH]1]

To a stirred solution of 6-([1,1′-biphenyl]-4-yl)-5-chloropyridine-2,3-diamine (2.0 g, 6.75 mmol, 1.0 eq) in THF (10 Vol) at room temperature, was added DIPEA (2.6 g, 20.25 mmol, 3.0 eq) followed by Thio-CDI (1.8 g, 10.13 mmol, 1.5 eq) and stirred at 70° C. for 2 h. After completion of reaction by TLC, the reaction mixture was quenched with 2N HCl solution (50 mL) and extracted with EtOAc (2× 40 mL). The combined organic layer was dried over Na₂SO₄, filtered and concentrated under reduced pressure to afford 5-([1,1′-biphenyl]-4-yl)-6-chloro-1,3-dihydro-2H-imidazo[4,5-b]pyridine-2-thione as a yellow solid (2.5 g, Yield: Crude). TLC system: EtOAc/Hexane (50:50), R_{f} value: ˜0.4; LCMS (m/z): 673.32 (M+H)⁺.

### Synthesis of 5-([1,1′-biphenyl]-4-yl)-6-chloro-2-(methylthio)-1H-imidazo[4,5-b]pyridine

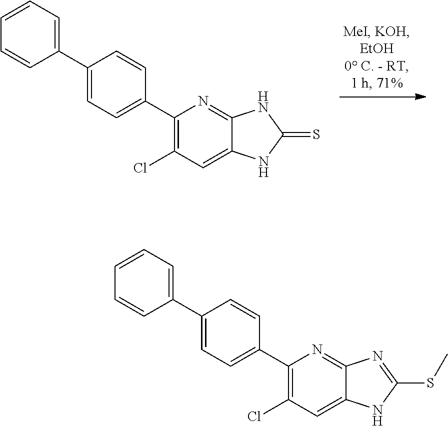 [SMILES: S=c1[nH]c2cc(Cl)c(-c3ccc(-c4ccccc4)cc3)nc2[nH]1 ; CSc1nc2nc(-c3ccc(-c4ccccc4)cc3)c(Cl)cc2[nH]1]

To a stirred solution of 5-([1,1′-biphenyl]-4-yl)-6-chloro-1,3-dihydro-2H-imidazo[4,5-b]pyridine-2-thione (2.3 g, 6.80 mmol, 1.0 eq) in EtOH (20 Vol) at 0° C., was added KOH (457 mg, 8.16 mmol, 1.2 eq) followed by MeI (965 mg, 6.80 mmol, 1.0 eq) and stirred at room temperature for 1 h. After completion of reaction by TLC, the reaction mixture was quenched with 2N HCl solution (15 mL), diluted with water (30 mL) and extracted with EtOAc (2×30 mL). The combined organic layer was dried over Na₂SO₄, filtered and concentrated under reduced pressure. The crude product was purified by silica gel [60-120 mesh] column chromatography [elution with 40% EtOAc in petroleum ether] to afford 5-([1,1′-biphenyl]-4-yl)-6-chloro-2-(methylthio)-1H-imidazo[4,5-b]pyridine as a yellow solid (1.7 g, Yield: 71%). TLC system: EtOAc/Hexane (70:30), R_{f} value: ˜0.4; LCMS (m/z): 352.16 (M+H)⁺; ¹H NMR (400 MHz, CDCl₃) δ: 8.12 (s, 1H), 7.80 (d, J=8.4 Hz, 2H), 7.72 (d, J=8.0 Hz, 2H), 7.66 (d, J=7.2 Hz, 2H), 7.52-7.41 (m, 2H), 7.32-7.26 (m, 1H), 2.79 (s, 3H).

### Synthesis of 5-([1,1′-biphenyl]-4-yl)-6-chloro-2-(methylsulfonyl)-1H-imidazo[4,5-b]pyridine

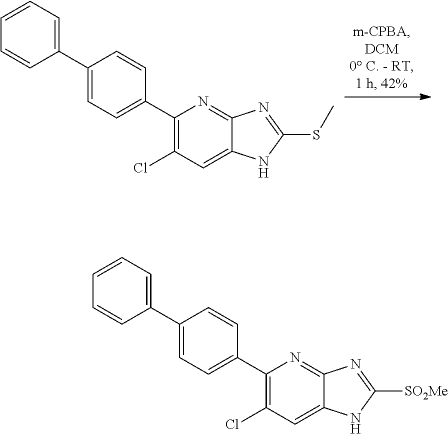 [SMILES: CSc1nc2nc(-c3ccc(-c4ccccc4)cc3)c(Cl)cc2[nH]1 ; CS(=O)(=O)c1nc2nc(-c3ccc(-c4ccccc4)cc3)c(Cl)cc2[nH]1]

To a stirred solution of 5-([1,1′-biphenyl]-4-yl)-6-chloro-2-(methylsulfonyl)-1H-imidazo[4,5-b]pyridine (1.2 g, 3.41 mmol, 1.0 eq) in DCM (10 Vol) at 0° C., was added m-CPBA (1.46 g, 8.52 mmol, 2.5 eq) and stirred at room temperature for 1 h. After completion of reaction by TLC, the reaction mixture was diluted with water (30 mL) and extracted with EtOAc (2×30 mL). The combined organic layer was washed with NaHCO₃ solution, dried over Na₂SO₄, filtered and concentrated under reduced pressure to afford crude product. The crude product was purified by silica gel [100-200 mesh] column chromatography [elution with 50% EtOAc in petroleum ether] to afford 5-([1,1′-biphenyl]-4-yl)-6-chloro-2-(methylsulfonyl)-1H-imidazo[4,5-b]pyridine as yellow solid (550 mg, Yield: 42%). TLC system: EtOAc/Hexane (50:50), R_{f} value: ˜0.5; LCMS (m/z): 384.21 (M+H)⁺; ¹H NMR (400 MHz, CDCl₃) δ: 8.30 (bs, 1H), 8.08-8.07 (s, 1H), 7.80 (d, J=8.0 Hz, 2H), 7.72 (d, J=8.0 Hz, 2H), 7.66 (d, J=7.2 Hz, 2H), 7.52-7.46 (m, 2H), 7.42-7.36 (m, 1H), 3.45 (s, 3H).

### Synthesis of 5-([1,1′-biphenyl]-4-yl)-6-chloro-2-(methylsulfonyl)-1-((2-(trimethylsilyl)ethoxy)methyl)-1H-imidazo[4,5-b]pyridine

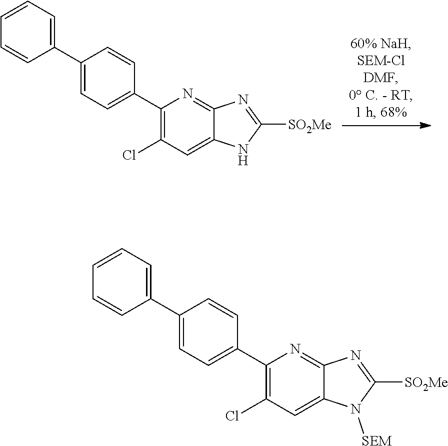 [SMILES: CS(=O)(=O)c1nc2nc(-c3ccc(-c4ccccc4)cc3)c(Cl)cc2[nH]1 ; C[Si](C)(C)CCOCn1c(S(C)(=O)=O)nc2nc(-c3ccc(-c4ccccc4)cc3)c(Cl)cc21]

To a stirred solution of 5-([1,1′-biphenyl]-4-yl)-6-chloro-2-(methylsulfonyl)-1H-imidazo[4,5-b]pyridine (500 mg, 1.30 mmol, 1.0 eq) in DMF (10 Vol) at 0° C., was added 60% NaH (62 mg, 1.56 mmol, 1.2 eq) and stirred for 30 min. Then SEM-Cl (261 mg, 1.56 mmol, 1.2 eq) was added to reaction mixture and stirred at room temperature for 1 h. After completion of reaction by TLC, the reaction mixture was quenched with water (20 mL) and extracted with EtOAc (2×20 mL). The combined organic layer was dried over Na₂SO₄, filtered and concentrated under reduced pressure to afford crude product. The crude product was purified by silica gel [60-120] column chromatography [elution with 20% EtOAc in petroleum ether] to afford 5-([1,1′-biphenyl]-4-yl)-6-chloro-2-(methylsulfonyl)-1-((2-(trimethylsilyl)ethoxy)methyl)-1H-imidazo[4,5-b]pyridine as a yellow gum (500 mg, Yield: 68%). TLC system: EtOAc/Hexane (30:70), R_{f} value: ˜0.6; LCMS (m/z): 514.28 (M+H)⁺.

### Synthesis of 6-((5-([1,1′-biphenyl]-4-yl)-6-chloro-1-((2-(trimethylsilyl)ethoxy)methyl)-1H-imidazo[4,5-b]pyridin-2-yl)oxy)benzo[c][1,2]oxaborol-1(3H)-ol

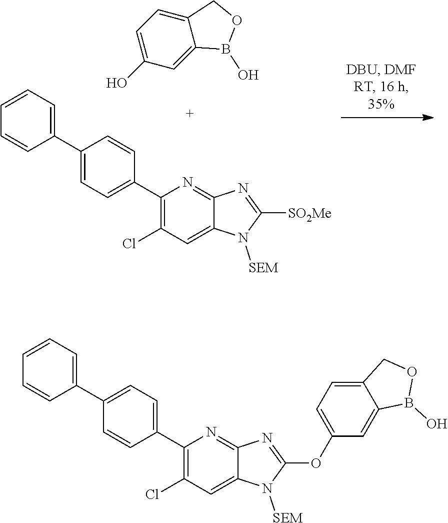 [SMILES: OB1OCc2ccc(O)cc21 ; C[Si](C)(C)CCOCn1c(S(C)(=O)=O)nc2nc(-c3ccc(-c4ccccc4)cc3)c(Cl)cc21 ; C[Si](C)(C)CCOCn1c(Oc2ccc3c(c2)B(O)OC3)nc2nc(-c3ccc(-c4ccccc4)cc3)c(Cl)cc21]

To a stirred solution of 5-([1,1′-biphenyl]-4-yl)-6-chloro-2-(methylsulfonyl)-1-((2-(trimethylsilyl)ethoxy)methyl)-1H-imidazo[4,5-b]pyridine (500 mg, 0.97 mmol, 1.0 eq) in DMF (5 Vol) at room temperature, was added DBU (442 mg, 2.91 mmol, 3.0 eq) followed by benzo[c][1,2]oxaborole-1,6 (3H)-diol (175 mg, 1.17 mmol, 1.2 eq) and stirred at room temperature for 16 h. After completion of reaction by TLC, the reaction mixture was concentrated under reduced pressure and purified by silica gel [60-120 mesh] column chromatography [elution with 5% MeOH in DCM] to afford semi-pure material. Re-repurified by reverse phase column chromatography [elution with 48% ACN in 0.1% FA in H2O] to afford 6-((5-([1,1′-biphenyl]-4-yl)-6-chloro-1-((2-(trimethylsilyl)ethoxy)methyl)-1H-imidazo[4,5-b]pyridin-2-yl)oxy)benzo[c][1,2]oxaborol-1(3H)-ol as a brown gum (200 mg, Yield: 35%). TLC system: EtOAc/Hexane (100:00), R_{f} value: ˜0.3; LCMS (m/z): 584.34 (M+H)⁺.

### Synthesis of 6-((5-([1,1′-biphenyl]-4-yl)-6-chloro-1H-imidazo[4,5-b]pyridin-2-yl)oxy)benzo[c][1,2]oxaborol-1(3H)-ol

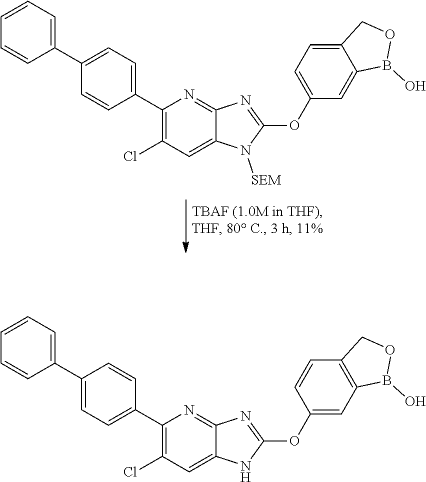 [SMILES: C[Si](C)(C)CCOCn1c(Oc2ccc3c(c2)B(O)OC3)nc2nc(-c3ccc(-c4ccccc4)cc3)c(Cl)cc21 ; OB1OCc2ccc(Oc3nc4nc(-c5ccc(-c6ccccc6)cc5)c(Cl)cc4[nH]3)cc21]

To a stirred solution of 6-((5-([1,1′-biphenyl]-4-yl)-6-chloro-1-((2-(trimethylsilyl)ethoxy)methyl)-1H-imidazo[4,5-b]pyridin-2-yl)oxy)benzo[c][1,2]oxaborol-1(3H)-ol (200 mg, 0.34 mmol, 1.0 eq) in THF (10 Vol) at 0° C., was added TBAF (1.0 M in THF) (2.0 mL, 10 Vol) and stirred at 80° C. for 3 h. After completion of reaction by TLC, the reaction mixture was quenched with NH₄Cl solution (10 mL) and extracted with EtOAc (2×10 mL). The combined organic layer was dried over Na₂SO₄, filtered and concentrated under reduced pressure to afford crude product. The crude product was purified by reverse phase column chromatography [elution with 25% ACN in 0.1% FA in H₂O] followed by Prep-HPLC and collected fractions were frozen, lyophilized to afford 6-((5-([1,1′-biphenyl]-4-yl)-6-chloro-1H-imidazo[4,5-b]pyridin-2-yl)oxy)benzo[c][1,2]oxaborol-1(3H)-ol white solid (17 mg, Yield: 11%). TLC system: MeOH/DCM (20:80), R_{f} value: ˜0.4; LCMS (m/z): 454.24 (M+H)⁺; ¹H NMR (400 MHz, DMSO-d₆) δ: 13.27 (bs, 1H), 9.32 (s, 1H), 8.00 (s, 1H), 7.80-7.73 (m, 7H), 7.54-7.49 (m, 4H), 7.42-7.38 (m, 1H), 5.04 (s, 2H).

Prep-HPLC condition: Column Gemini nx C-18 (250*21.2 mm) 5μ; Buffer MP-(A): 0.2% FA in H2O, MP-(B): ACN:WATER (80:20); Gradient (T/% B) 0/70, 10/90, 15/98, 25/98; Flow Rate 13 ml/min; Solubility ACN, WATER, THF

### SYNTHETIC EXAMPLE 63

### Synthesis of Compound I-75

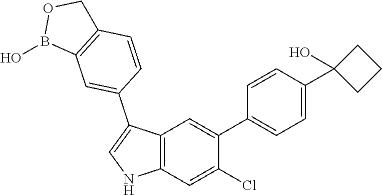 [SMILES: OB1OCc2ccc(-c3c[nH]c4cc(Cl)c(-c5ccc(C6(O)CCC6)cc5)cc34)cc21]

### Synthesis of 5-bromo-6-chloro-3-iodo-1H-indole

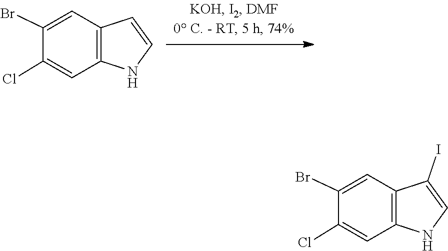 [SMILES: Clc1cc2[nH]ccc2cc1Br ; Clc1cc2[nH]cc(I)c2cc1Br]

To a stirred solution of 5-bromo-6-chloro-1H-indole (7.0 g, 30.43 mmol, 1.0 eq) in DMF (10 Vol) at 0° C., was added KOH (4.26 g, 76.08 mmol, 2.5 eq) followed by 12 (7.73 g, 30.43 mmol, 1.0 eq) and stirred at room temperature for 5 h. After completion of reaction by TLC, the reaction mixture was diluted with water (70 mL) and extracted with EtOAc (2×70 mL). The combined organic layer was dried over Na₂SO₄, filtered and concentrated under reduced pressure. The crude product was purified by silica gel [100-200] column chromatography [elution with 10% EtOAc in petroleum ether] to afford 5-bromo-6-chloro-3-iodo-1H-indole as an off white solid (8.0 g, Yield: 74%). TLC system: EtOAc/Hexane (20:80), R_{f} value: ˜0.5; ¹H NMR (400 MHz, CDCl₃) δ: 8.36 (br s, 1H), 7.69 (s, 1H), 7.49 (s, 1H), 7.27 (d, J=2.4 Hz, 1H).

### Synthesis of 5-bromo-6-chloro-3-iodo-1-((2-(trimethylsilyl)ethoxy)methyl)-1H-indole

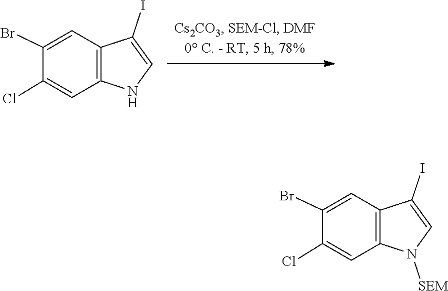 [SMILES: Clc1cc2[nH]cc(I)c2cc1Br ; C[Si](C)(C)CCOCn1cc(I)c2cc(Br)c(Cl)cc21]

To a stirred solution of 5-bromo-6-chloro-3-iodo-1H-indole (8.0 g, 22.53 mmol, 1.0 eq) in DMF (10 Vol) at 0° C., was added Cs₂CO₃ (14.68 g, 45.06 mmol, 2.0 eq) followed by SEM-Cl (5.64 g, 33.8, 1.5 eq) and stirred at room temperature for 2 h. After completion of reaction by TLC, the reaction mixture was diluted with water (100 mL) and extracted with EtOAc (2×100 mL). The combined organic layer was dried over Na₂SO₄, filtered and concentrated under reduced pressure to afford crude product. The crude product was purified by silica gel [100-200] column chromatography [elution with 20% EtOAc in petroleum ether] to afford 5-bromo-6-chloro-3-iodo-1-((2-(trimethylsilyl)ethoxy)methyl)-1H-indole as a yellow gum (8.5 g, Yield: 78%). TLC system: EtOAc/Hexane (20:80), R_{f} value: ˜0.6; ¹H NMR (400 MHz, CDCl₃) δ: 7.74 (s, 1H), 7.65 (s, 1H), 7.27 (s, 1H), 5.43 (s, 2H), 3.54-3.48 (m, 2H), 0.97-0.91 (m, 2H), 0.041 (s, 9H).

### Synthesis of 4-(5-bromo-6-chloro-1-((2-(trimethylsilyl)ethoxy)methyl)-1H-indol-3-yl)-2-hydroxybenzaldehyde

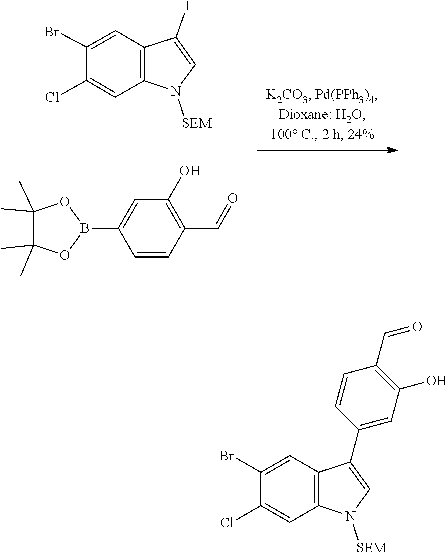 [SMILES: C[Si](C)(C)CCOCn1cc(I)c2cc(Br)c(Cl)cc21 ; CC1(C)OB(c2ccc(C=O)c(O)c2)OC1(C)C ; C[Si](C)(C)CCOCn1cc(-c2ccc(C=O)c(O)c2)c2cc(Br)c(Cl)cc21]

In a sealed tube to a degassed solution of 5-bromo-6-chloro-3-iodo-1-((2-(trimethylsilyl)ethoxy)methyl)-1H-indole (2×3 g, 12.37 mmol, 1.0 eq) and 2-hydroxy-4-(4,4,5,5-tetramethyl-1,3,2-dioxaborolan-2-yl)benzaldehyde (4.6 g, 18.56 mmol, 1.5 eq) in Dioxane:H₂O (6:1, 40 Vol) at room temperature, was added K₂CO₃ (8.53 g, 61.85 mmol, 5.0 eq) followed by Pd(PPh₃)₄ (1.43 g, 1.24 mmol, 0.1 eq) and stirred at 100° C. for 2 h. After completion of reaction by TLC, the reaction mixture was diluted with water (100 mL) and extracted with EtOAc (2×100 mL). The combined organic layer was dried over Na₂SO₄, filtered and concentrated under reduced pressure to afford crude product. The crude product was purified by silica gel [100-200] column chromatography [elution with 15% EtOAc in petroleum ether] to afford 4-(5-bromo-6-chloro-1-((2-(trimethylsilyl)ethoxy)methyl)-1H-indol-3-yl)-2-hydroxybenzaldehyde as a yellow solid (1.5 g, Yield: 24%). TLC system: EtOAc/Hexane (20:80), R_{f} value: ˜0.4; LCMS (m/z): 480.37 (M+H)⁺.

### Synthesis of 4-(6-chloro-5-(4-(1-hydroxycyclobutyl)phenyl)-1-((2-(trimethylsilyl)ethoxy)methyl)-1H-indol-3-yl)-2-hydroxybenzaldehyde

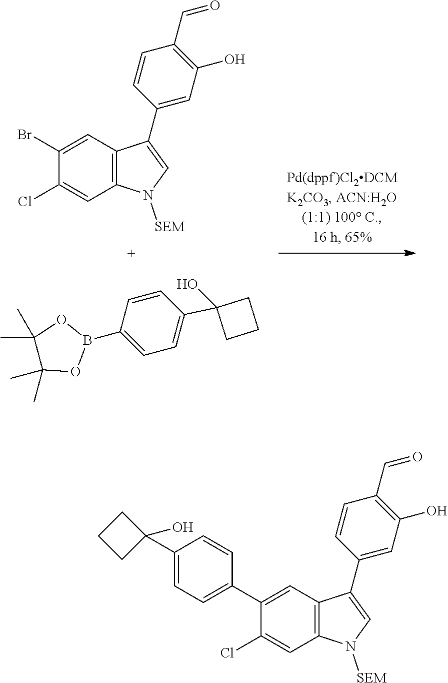 [SMILES: C[Si](C)(C)CCOCn1cc(-c2ccc(C=O)c(O)c2)c2cc(Br)c(Cl)cc21 ; CC1(C)OB(c2ccc(C3(O)CCC3)cc2)OC1(C)C ; C[Si](C)(C)CCOCn1cc(-c2ccc(C=O)c(O)c2)c2cc(-c3ccc(C4(O)CCC4)cc3)c(Cl)cc21]

In a sealed tube, to a degassed solution of 4-(5-bromo-6-chloro-1-((2-(trimethylsilyl)ethoxy)methyl)-1H-indol-3-yl)-2-hydroxybenzaldehyde (1.5 g, 3.12 mmol, 1.0 eq) and 1-(4-(4,4,5,5-tetramethyl-1,3,2-dioxaborolan-2-yl)phenyl)cyclobutan-1-ol (1.03 g, 3.75 mmol, 1.2 eq) in ACN:H₂O (1:1, 10 Vol) at room temperature, was added K₂CO₃ (1.29 g, 9.36 mmol, 3.0 eq) followed by Pd(dppf)Cl₂·DCM (253 mg, 0.31 mmol, 0.1 eq) and stirred at 100° C. for 16 h. After completion of reaction by TLC, the reaction mixture was diluted with water (30 mL) and extracted with EtOAc (2×30 mL). The combined organic layer was dried over Na₂SO₄, filtered and concentrated under reduced pressure to afford crude product. The crude product was purified by silica gel [100-200] column chromatography [elution with 15% EtOAc in petroleum ether] to afford 4-(6-chloro-5-(4-(1-hydroxycyclobutyl)phenyl)-1-((2-(trimethylsilyl)ethoxy)methyl)-1H-indol-3-yl)-2-hydroxybenzaldehyde as a brown solid (1.1 g, Yield: 65%). TLC system: EtOAc/Hexane (30:70), R_{f} value: ˜0.5; LCMS (m/z): 546.21 (M−H)⁻.

### Synthesis of 5-(6-chloro-5-(4-(1-hydroxycyclobutyl)phenyl)-1-((2-(trimethylsilyl)ethoxy)methyl)-1H-indol-3-yl)-2-formylphenyl trifluoromethanesulfonate

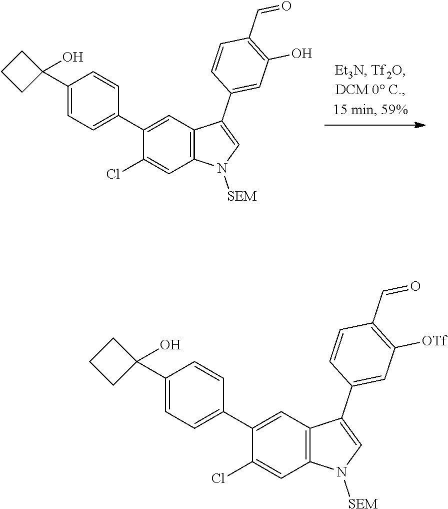 [SMILES: C[Si](C)(C)CCOCn1cc(-c2ccc(C=O)c(O)c2)c2cc(-c3ccc(C4(O)CCC4)cc3)c(Cl)cc21 ; C[Si](C)(C)CCOCn1cc(-c2ccc(C=O)c(OS(=O)(=O)C(F)(F)F)c2)c2cc(-c3ccc(C4(O)CCC4)cc3)c(Cl)cc21]

To a stirred solution of 4-(6-chloro-5-(4-(1-hydroxycyclobutyl)phenyl)-1-((2-(trimethylsilyl)ethoxy)methyl)-1H-indol-3-yl)-2-hydroxybenzaldehyde (1.1 g, 2.01 mmol, 1.0 eq) in DCM (20 Vol) at 0° C., was added Et₃N (3.66 g, 6.02 mmol, 3.0 eq) followed by Tf₂O (0.85 g, 3.01 mmol, 1.5 eq) and stirred for 15 min. After completion of reaction by TLC, the reaction mixture was diluted with water (30 mL) and extracted with EtOAc (2×30 mL). The combined organic layer was dried over Na₂SO₄, filtered and concentrated under reduced pressure to afford crude product. The crude product was purified by silica gel [100-200] column chromatography [elution with 20% EtOAc in petroleum ether] to afford 5-(6-chloro-5-(4-(1-hydroxycyclobutyl)phenyl)-1-((2-(trimethylsilyl)ethoxy)methyl)-1H-indol-3-yl)-2-formylphenyl trifluoromethanesulfonate as a yellow solid (800 mg, Yield: 59%). TLC system: EtOAc/Hexane (30:70), R_{f} value: ˜0.4; LCMS (m/z): 662.32 (M−17)+.

### Synthesis of 4-(6-chloro-5-(4-(1-hydroxycyclobutyl)phenyl)-1-((2-(trimethylsilyl)ethoxy)methyl)-1H-indol-3-yl)-2-(4,4,5,5-tetramethyl-1,3,2-dioxaborolan-2-yl)benzaldehyde

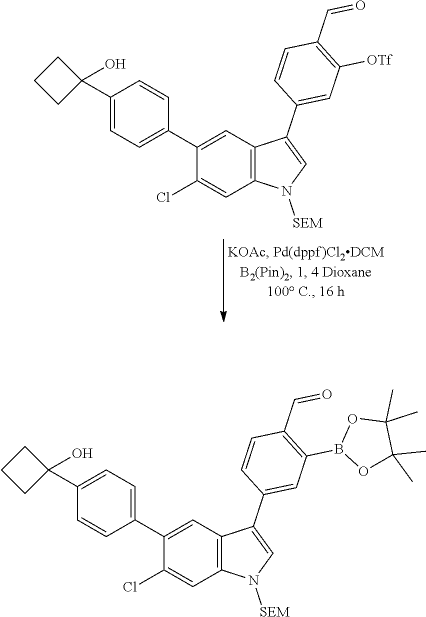 [SMILES: C[Si](C)(C)CCOCn1cc(-c2ccc(C=O)c(OS(=O)(=O)C(F)(F)F)c2)c2cc(-c3ccc(C4(O)CCC4)cc3)c(Cl)cc21 ; CC1(C)OB(c2cc(-c3cn(COCC[Si](C)(C)C)c4cc(Cl)c(-c5ccc(C6(O)CCC6)cc5)cc34)ccc2C=O)OC1(C)C]

In a sealed tube to a degassed solution of 5-(6-chloro-5-(4-(1-hydroxycyclobutyl)phenyl)-1-((2-(trimethylsilyl)ethoxy)methyl)-1H-indol-3-yl)-2-formylphenyl trifluoromethanesulfonate (800 mg, 1.18 mmol, 1.0 eq) and B₂Pin₂ (448 mg, 1.76 mmol, 1.5 eq) in Dioxane (20 Vol) at room temperature, was added KOAc (289 mg, 2.95 mmol, 2.5 eq) followed by Pd(dppf)Cl₂·DCM (96 mg, 0.12 mmol, 0.1 eq) and stirred at 100° C. for 16 h. After completion of reaction by TLC, the reaction mixture was diluted with water (20 mL) and extracted with EtOAc (2×20 mL). The combined organic layer was dried over Na₂SO₄, filtered and concentrated under reduced pressure to afford crude product. The crude product was purified by reverse phase column chromatography [elution with 90% ACN in 0.1% FA in H₂O] to afford 4-(6-chloro-5-(4-(1-hydroxycyclobutyl)phenyl)-1-((2-(trimethylsilyl)ethoxy)methyl)-1H-indol-3-yl)-2-(4,4,5,5-tetramethyl-1,3,2-dioxaborolan-2-yl)benzaldehyde as a brown solid (300 mg, semi-pure). TLC system: EtOAc/Hexane (50:50), R_{f} value: ˜0.6; LCMS (m/z): 658.61 (M+H)⁺;

### Synthesis of 6-(6-chloro-5-(4-(1-hydroxycyclobutyl)phenyl)-1-((2-(trimethylsilyl)ethoxy)methyl)-1H-indol-3-yl)benzo[c][1,2]oxaborol-1(3H)-ol

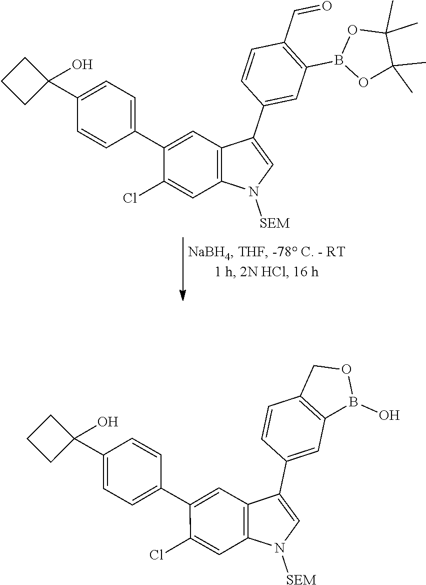 [SMILES: CC1(C)OB(c2cc(-c3cn(COCC[Si](C)(C)C)c4cc(Cl)c(-c5ccc(C6(O)CCC6)cc5)cc34)ccc2C=O)OC1(C)C ; C[Si](C)(C)CCOCn1cc(-c2ccc3c(c2)B(O)OC3)c2cc(-c3ccc(C4(O)CCC4)cc3)c(Cl)cc21]

To a stirred solution of 4-(6-chloro-5-(4-(1-hydroxycyclobutyl)phenyl)-1-((2-(trimethylsilyl)ethoxy)methyl)-1H-indol-3-yl)-2-(4,4,5,5-tetramethyl-1,3,2-dioxaborolan-2-yl)benzaldehyde (0.4 g, 0.72 mmol, 1.0 eq) in THF (20 Vol) at −78° C., was added NaBH₄ (41 mg, 1.07 mmol, 1.5 eq) and stirred at room temperature for 1 h. After completion of reaction by TLC, the reaction mixture was quenched with 2N HCl solution (5 Vol) and stirred at room temperature for 16 h. Then reaction mixture was diluted with water (20 mL) and extracted with EtOAc (2×20 mL). The combined organic layer was dried over Na₂SO₄, filtered and concentrated under reduced pressure to afford 6-(6-chloro-5-(4-(1-hydroxycyclobutyl)phenyl)-1-((2-(trimethylsilyl)ethoxy)methyl)-1H-indol-3-yl)benzo[c][1,2]oxaborol-1(3H)-ol as a brown solid (350 mg, semi-pure). TLC system: EtOAc/Hexane (50:50), R_{f} value: ˜0.3; LCMS (m/z): 542.48 (M−17)⁺:

### Synthesis of 6-(6-chloro-5-(4-(1-hydroxycyclobutyl)phenyl)-1H-indol-3-yl)benzo[c][1,2]oxaborol-1(3H)-ol

To a stirred solution of 6-(6-chloro-5-(4-(1-hydroxycyclobutyl)phenyl)-1-((2-(trimethylsilyl)ethoxy)methyl)-1H-indol-3-yl)benzo[c][1,2]oxaborol-1(3H)-ol (340 mg, 0.61 mmol, 1.0 eq) in THF (10 Vol) at 0° C., was added TBAF (1.0 M in THF) (10 Vol) and stirred at 80° C. for 2 h. After completion of reaction by TLC, the reaction mixture was quenched with NH₄Cl solution (20 mL) and extracted with EtOAc (2×20 mL). The combined organic layer was dried over Na₂SO₄, filtered and concentrated under reduced pressure to afford crude product. The crude product was purified by reverse phase column chromatography [elution with 55% ACN in 0.1% FA in H₂O] followed by Prep-HPLC and collected fractions were freeze, lyophilized to afford 6-(6-chloro-5-(4-(1-hydroxycyclobutyl)phenyl)-1H-indol-3-yl)benzo[c][1,2]oxaborol-1(3H)-ol as white solid (35 mg, Yield: 5.5% in 3 steps). TLC system: MeOH/DCM (100:00), R_{f} value: ˜0.3; LCMS (m/z): 541.24 (M+H)⁺; ¹H NMR (400 MHz, DMSO-d₆) δ: 11.54 (s, 1H), 9.24 (s, 1H), 8.06 (s, 1H), 7.82-7.77 (m, 3H), 7.63 (s, 1H), 7.56 (d, J=8.4 Hz, 2H), 7.46-7.43 (m, 3H), 5.53 (s, 1H), 5.00 (s, 1H), 2.49-2.42 (m, 2H), 2.34-2.26 (m, 2H), 1.96-1.93 (m, 1H), 1.70-1.67 (m, 1H).

Prep-HPLC condition: Column YMC C-18 (250.19 mm) 5u Buffer MP-(A): 0.1% FA in WATER MP-(B): 0.1% FA in ACN Gradient (T/% B) 0/55, 10/75, 15/75, 20/95, 25/95 Flow Rate 13 ml/min Solubility ACN&WATER.

### SYNTHETIC EXAMPLE 64

### Synthesis of Compound I-76

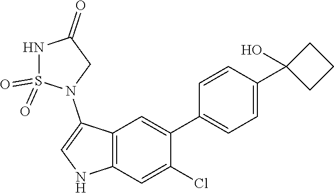 [SMILES: O=C1CN(c2c[nH]c3cc(Cl)c(-c4ccc(C5(O)CCC5)cc4)cc23)S(=O)(=O)N1]

### Synthesis of 5-bromo-6-chloro-1-tosyl-1H-indole

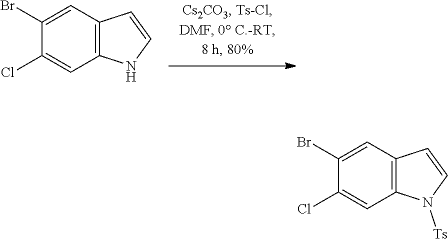 [SMILES: Clc1cc2[nH]ccc2cc1Br ; Cc1ccc(S(=O)(=O)n2ccc3cc(Br)c(Cl)cc32)cc1]

To a stirred solution of 5-bromo-6-chloro-1H-indole (3 g, 13.1 mmol, 1.0 eq) in DMF (30 mL, 10 Vol) at 0° C., was added Cs₂CO₃ (10.59 g, 32.7 mmol, 2.5 eq) and stirred at 0° C. for 10 min. Later, added tosyl chloride (6.19 g, 32.7 mmol, 2.5 eq) and stirred at room temperature for 8 h. After completion of reaction by TLC, the reaction mixture was diluted with water (70 mL) and extracted with EtOAc (2×100 mL). The combined organic layer was dried over Na₂SO₄, filtered and concentrated under reduced pressure to afford 5-bromo-6-chloro-1-tosyl-1H-indole as off white solid (4.0 g, Yield: 80%). TLC system: 20% EtOAc/petroleum ether (20:80), R_{f} value: ˜0.4; ¹H NMR (400 MHz, CDCl₃) δ: 8.12 (s, 1H), 7.77-7.73 (m, 3H), 7.55 (d, J=3.6 Hz, 1H), 6.57 (d, J=3.6 Hz, 1H), 2.37 (s, 3H). 2 aromatic protons merging with CHCl₃ residual peak.

### Synthesis of 5-bromo-6-chloro-3-nitro-1-tosyl-1H-indole

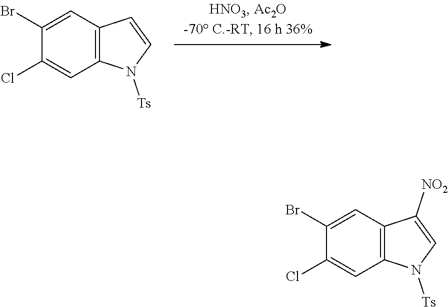 [SMILES: Cc1ccc(S(=O)(=O)n2ccc3cc(Br)c(Cl)cc32)cc1 ; Cc1ccc(S(=O)(=O)n2cc([N+](=O)[O-])c3cc(Br)c(Cl)cc32)cc1]

To a stirred solution of 5-bromo-6-chloro-1-tosyl-1H-indole (4 g, 10.4 mmol, 1.0 eq) in Ac₂O (80 mL, 20 Vol) at −70° C. was added HNO₃ (5.6 mL, 135.4 mol, 13 eq) dropwise (pre-mixed solution of Ac₂O) and the reaction mixture was allowed to room temperature, stirred for 16 h. After completion of reaction by TLC, the reaction mixture poured into ice cold water and filtered, washed with n-pentane to afford 5-bromo-6-chloro-3-nitro-1-tosyl-1H-indole as pale yellow color solid (1.6 g, Yield: 36%). TLC system: EtOAC/Hexane (20:80), R_{f} value: ^{˜}0.2; ¹H NMR (400 MHz, DMSO-d₆) δ: 9.22 (s, 1H), 8.39 (s, 1H), 8.25-8.22 (m, 3H), 7.51 (dd, J=8.0 Hz, 2H), 2.38 (s, 3H).

### Synthesis of 5-bromo-6-chloro-1-tosyl-1H-indol-3-amine

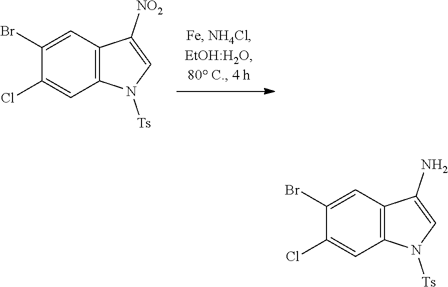 [SMILES: Cc1ccc(S(=O)(=O)n2cc([N+](=O)[O-])c3cc(Br)c(Cl)cc32)cc1 ; Cc1ccc(S(=O)(=O)n2cc(N)c3cc(Br)c(Cl)cc32)cc1]

To a stirred solution of 5-bromo-6-chloro-3-nitro-1-tosyl-1H-indole (1.8 g, 4.20 mmol, 1 eq) in EtOH:H₂O (1:1) (36 mL) at room temperature, was added Fe powder (1.17 g, 21.02 mmol, 5 eq) and NH₄Cl (1.11 g, 21.02 mmol, 5 eq). The resulting reaction mixture was heated to 80° C., stirred for 4 h. After completion of reaction by TLC, reaction mixture was filtered through a pad of diatomaceous earth (e.g., Celite®), and washed with EtOAc (200 mL). Filtrate concentrated, diluted with water (50 mL) and extracted with EtOAc (2×200 mL). Combined organic layer was dried over Na₂SO₄, filtered and concentrated under reduced pressure to afford 5-bromo-6-chloro-1-tosyl-1H-indol-3-amine as brown solid (1.7 g, Crude). TLC system: EtOAc/Hexane (30:70), R_{f} value: ^{˜}0.1; LCMS (m/z): 399.26 (M+H)⁺.

### Synthesis of ethyl (5-bromo-6-chloro-1-tosyl-1H-indol-3-yl)glycinate

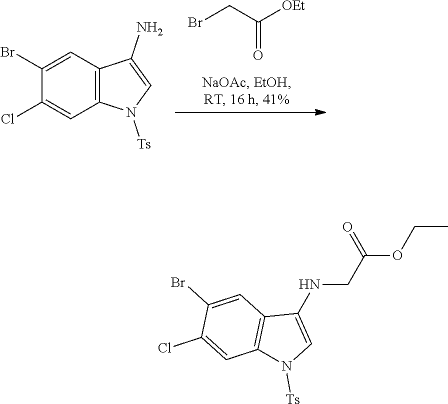 [SMILES: Cc1ccc(S(=O)(=O)n2cc(N)c3cc(Br)c(Cl)cc32)cc1 ; CCOC(=O)CBr ; CCOC(=O)CNc1cn(S(=O)(=O)c2ccc(C)cc2)c2cc(Cl)c(Br)cc12]

To a stirred solution of 5-bromo-6-chloro-1-tosyl-1H-indol-3-amine (1.2 g, 3.01 mmol, 1.0 eq), ethyl 2-bromoacetate (0.4 mL, 3.61 mmol, 1.2 eq) in EtOH (24 mL, 20 Vol) at RT, was added NaOAc (495 mg, 6.03 mmol, 2 eq), and stirred for 16 h. After completion of reaction by TLC, the reaction mixture was diluted with ice cold water (50 mL) and extracted with EtOAc (2×100 mL). The combined organic layer was dried over Na₂SO₄, filtered and concentrated under reduced to afford crude. The crude material was purified by silica gel (100-200) column chromatography [elution with 5-10% EtOAc/Hexane] to afford ethyl (5-bromo-6-chloro-1-tosyl-1H-indol-3-yl)glycinate as brown semi solid (600 mg, Yield: 41%). TLC system: EtOAc/Hexane (30:70) (2-Times elution), R_{f} value: ^{˜}0.4; LCMS (m/z): 485.30 (M+H)⁺.

### Synthesis of ethyl N-(5-bromo-6-chloro-1-tosyl-1H-indol-3-yl)-N-(N-(tert-butoxycarbonyl)sulfamoyl)glycinate

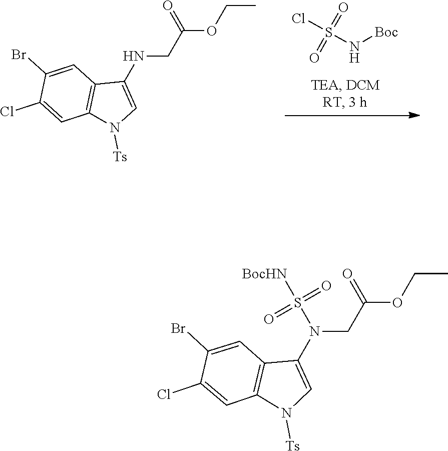 [SMILES: CCOC(=O)CNc1cn(S(=O)(=O)c2ccc(C)cc2)c2cc(Cl)c(Br)cc12 ; CC(C)(C)OC(=O)NS(=O)(=O)Cl ; CCOC(=O)CN(c1cn(S(=O)(=O)c2ccc(C)cc2)c2cc(Cl)c(Br)cc12)S(=O)(=O)NC(=O)OC(C)(C)C]

To a stirred solution of ethyl (5-bromo-6-chloro-1-tosyl-1H-indol-3-yl)glycinate (600 mg, 1.24 mmol, 1.0 eq) in DCM (12 mL, 10 Vol) at 0° C., added TEA (0.52 mL, 3.72 mmol, 3.0 eq), tert-butyl (chlorosulfonyl)carbamate (533 mg, 2.48 mmol, 2 eq) and stirred at room temperature for 3 h. After completion of reaction by TLC, the reaction mixture was diluted with ice cold water (15 mL) and extracted with EtOAc (2×30 mL). The combined organic layer was dried over Na₂SO₄, filtered and concentrated under reduced pressure to afford ethyl N-(5-bromo-6-chloro-1-tosyl-1H-indol-3-yl)-N-(N-(tert-butoxycarbonyl)sulfamoyl)glycinate as brown gummy (800 mg, Crude). TLC system: EtOAc/hexane (20:80), R_{f} value: ^{˜}0.2; LCMS (m/z): 662.09 (M−H)⁻.

### Synthesis of ethyl N-(5-bromo-6-chloro-1-tosyl-1H-indol-3-yl)-N-sulfamoylglycinate

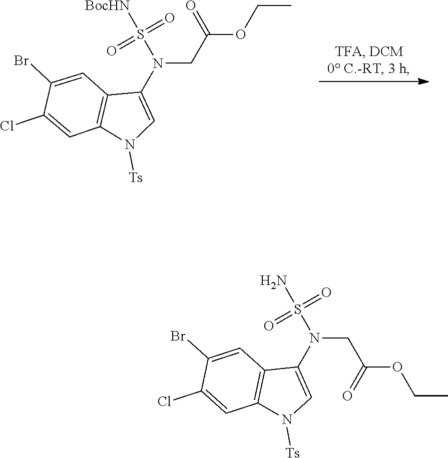 [SMILES: CCOC(=O)CN(c1cn(S(=O)(=O)c2ccc(C)cc2)c2cc(Cl)c(Br)cc12)S(=O)(=O)NC(=O)OC(C)(C)C ; CCOC(=O)CN(c1cn(S(=O)(=O)c2ccc(C)cc2)c2cc(Cl)c(Br)cc12)S(N)(=O)=O]

To a stirred solution of ethyl N-(5-bromo-6-chloro-1-tosyl-1H-indol-3-yl)-N-(N-(tert-butoxycarbonyl)sulfamoyl)glycinate (870 mg, 1.31 mmol, 1.0 eq) in DCM (17.5 mL, 20 Vol) at 0° C., was added TFA (4.35 mL, 5 Vol) and stirred at room temperature for 3 h. After completion of reaction by TLC, reaction mixture was concentrated, diluted with sat. NaHCO₃ (20 mL) and extracted with EtOAc (2×50 mL). The combined organic layer was dried over Na₂SO₄, filtered and concentrated under reduced to afford ethyl N-(5-bromo-6-chloro-1-tosyl-1H-indol-3-yl)-N-sulfamoylglycinate as brown gummy (700 mg, crude). TLC system: EtOAc/Hexane (30:70), R_{f} value: ^{˜}0.1; LCMS (m/z): 564.2 (M+H)⁺.

### Synthesis of 5-(5-bromo-6-chloro-1H-indol-3-yl)-1,2,5-thiadiazolidin-3-one 1,1-dioxide

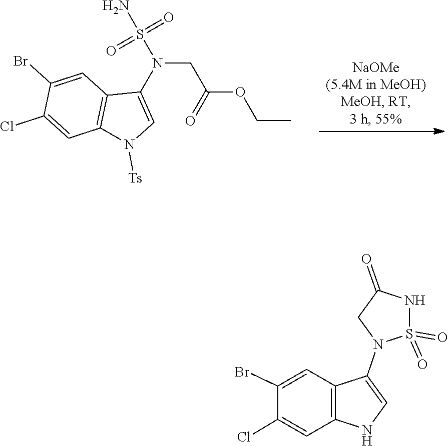 [SMILES: CCOC(=O)CN(c1cn(S(=O)(=O)c2ccc(C)cc2)c2cc(Cl)c(Br)cc12)S(N)(=O)=O ; O=C1CN(c2c[nH]c3cc(Cl)c(Br)cc23)S(=O)(=O)N1]

To a stirred solution of ethyl N-(5-bromo-6-chloro-1-tosyl-1H-indol-3-yl)-N-sulfamoylglycinate (Crude) (700 mg, 1.24 mmol, 1.0 eq) in MeOH (7 mL 10 vol) at 0° C., was added 5.4 M NaOMe in MeOH (0.7 mL, 1 vol) and stirred at RT for 3 h. After completion of reaction by Cr. LCMS, the reaction mixture concentrated under reduced pressure to afford crude compound (450 mg) as a brown gummy solid. The crude product was purified by reverse phase column chromatography (Grace) [elution with 18-22% of ACN in 0.1% FA in H₂O] and fractions were concentrated to afford 5-(5-bromo-6-chloro-1H-indol-3-yl)-1,2,5-thiadiazolidin-3-one 1,1-dioxide as a pale brown solid (250 mg, 55% in 3 steps). TLC system: MeOH/EtOAc (10:90), R_{f} value: ^{˜}0.05; LCMS (m/z): 361.99 (M−H)⁻. ¹H NMR (400 MHz, DMSO-d₆) δ: 11.45 (s, 1H), 8.12 (s, 1H), 7.65 (s, 1H), 7.55 (d, J=2.8 Hz, 1H), 4.37 (s, 2H).

### Synthesis of 5-(6-chloro-5-(4-(1-hydroxycyclobutyl)phenyl)-1H-indol-3-yl)-1,2,5-thiadiazolidin-3-one 1,1-dioxide

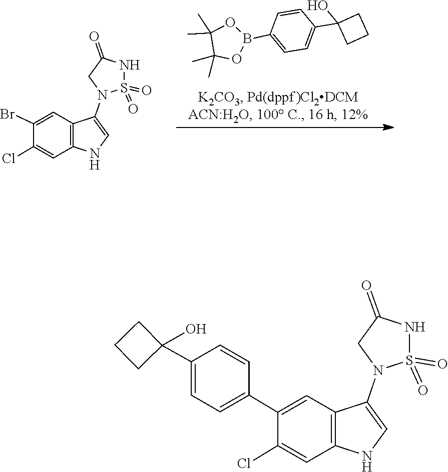 [SMILES: O=C1CN(c2c[nH]c3cc(Cl)c(Br)cc23)S(=O)(=O)N1 ; CC1(C)OB(c2ccc(C3(O)CCC3)cc2)OC1(C)C ; O=C1CN(c2c[nH]c3cc(Cl)c(-c4ccc(C5(O)CCC5)cc4)cc23)S(=O)(=O)N1]

In a sealed tube, to a degassed solution of 5-(5-bromo-6-chloro-1H-indol-3-yl)-1,2,5-thiadiazolidin-3-one 1,1-dioxide (200 mg, 0.55 mmol, 1.0 eq) and 1-(4-(4,4,5,5-tetramethyl-1,3,2-dioxaborolan-2-yl)phenyl)cyclobutan-1-ol (181 mg, 0.66 mmol, 1.5 eq) in ACN:H₂O (8:2, 2 mL) at room temperature, was added K₂CO₃ (230 mg, 1.65 mmol, 3.0 eq) followed by Pd(dppf)Cl₂·DCM (45 mg, 0.055 mmol, 0.1 eq) and stirred at 100° C. for 16 h. After completion of reaction by TLC, the reaction mixture was diluted with water (10 mL) and extracted with EtOAc (2×15 mL). The combined organic layer was dried over Na₂SO₄, filtered and concentrated under reduced pressure to afford crude product. The crude product was purified by reverse phase column chromatography (Grace) [elution with 30-35% of ACN in 0.1% FA in H₂O] and fractions were lyophilized to afford semi pure compound. Re-purified by Prep-HPLC and pure fractions were lyophilized to afford 5-(6-chloro-5-(4-(1-hydroxycyclobutyl)phenyl)-1H-indol-3-yl)-1,2,5-thiadiazolidin-3-one 1,1-dioxide as white solid (28 mg, Yield: 12%). TLC system: MeOH/EtOAc (20:80), R_{f} value: ˜0.1; LCMS (m/z): 430.16 (M−H)⁻. ¹H NMR (400 MHz, DMSO-d₆) δ: 11.04 (d, J=2 Hz, 1H), 7.85 (s, 1H), 7.54 (d, J=8.4 Hz, 2H), 7.48 (s, 1H), 7.38 (d, J=8.4 Hz, 2H), 7.30 (d, J=2.4 Hz, 1H), 5.51 (s, 1H), 4.00 (s, 2H), 2.48-2.42 (m, 2H), 2.33-2.26 (m, 2H), 1.96-1.90 (m, 1H), 1.72-1.67 (m, 1H).

Prep-HPLC condition: Column: YMC C-18 (250*19 mm) 5μ; Buffer: MP-(A): 10 mM abc in water, MP-(B): ACN; Gradient: 0/20,15/85, 25/95; Flow Rate: 15 ml/min; Solubility: ACN&Water.

### SYNTHETIC EXAMPLE 65

### Synthesis of Compound II-22

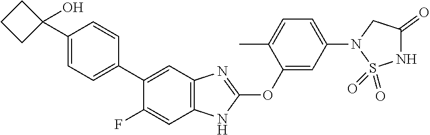 [SMILES: Cc1ccc(N2CC(=O)NS2(=O)=O)cc1Oc1nc2cc(-c3ccc(C4(O)CCC4)cc3)c(F)cc2[nH]1]

### Synthesis of 5-bromo-6-fluoro-1,3-dihydro-2H-benzo[d]imidazol-2-one

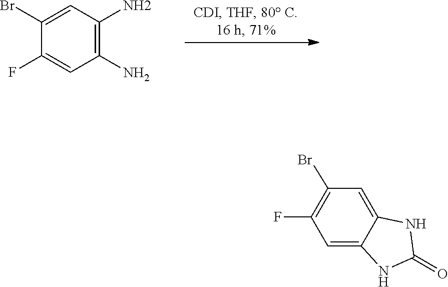 [SMILES: Nc1cc(F)c(Br)cc1N ; O=c1[nH]c2cc(F)c(Br)cc2[nH]1]

To a stirred solution of 4-bromo-5-chlorobenzene-1,2-diamine (5 g, 24.39 mmol, 1.0 eq) in THF (10 Vol) at room temperature, was added CDI (4.7 g, 29.26 mmol, 1.2 eq) and stirred at 80° C. for 16 h. After completion of reaction by TLC, the reaction mixture was quenched with water (50 mL), solid was filtered, washed with H₂O (60 mL) and collected solid was dried to afford crude Product. The Crude product was triturated with diethyl ether (50 mL), decanted and dried under reduced pressure to afford 5-bromo-6-fluoro-1,3-dihydro-2H-benzo[d]imidazol-2-one as brown solid (4 g, Yield: 71%). TLC system: EtOAc/Hexane (100:00), R_{f} value: ˜0.2; ¹H NMR (400 MHz, DMSO-d₆) δ: 10.93 (s, 1H), 10.78 (s, 1H), 7.11 (d, J=6 Hz, 1H), 6.96 (d, J=8.8 Hz, 1H).

### Synthesis of 5-bromo-2-chloro-6-fluoro-1H-benzo[d]imidazole

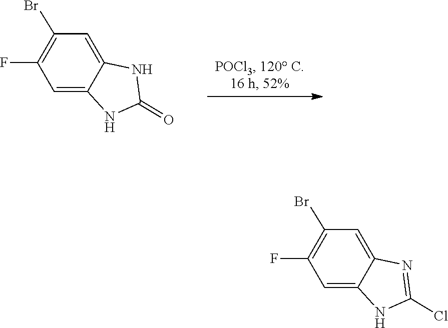 [SMILES: O=c1[nH]c2cc(F)c(Br)cc2[nH]1 ; Fc1cc2[nH]c(Cl)nc2cc1Br]

A solution of 5-bromo-6-fluoro-1,3-dihydro-2H-benzo[d]imidazol-2-one (2 g, 8.65 mmol, 1.0 eq) in POCl₃ (6 mL, 3 Vol) was stirred at 120° C. for 16 h. After completion of reaction by TLC, the reaction mixture was quenched with NaHCO₃ (50 mL), diluted with H₂O (50 mL) and extracted with EtOAc (2×50 mL). The combined organic layer was dried over Na₂SO₄, filtered and concentrated under reduced pressure. The crude product was purified by silica gel [100-200] column chromatography [elution with 30% EtOAc in petroleum ether] to afford 5-bromo-2-chloro-6-fluoro-1H-benzo[d]imidazole as pale brown solid (1.1 g, Yield: 52%). TLC system: EtOAc/Hexane (30:70), R_{f} value: ˜0.4; LCMS (m/z): 248.99 (M+H)⁺.

### Synthesis of 5-bromo-2-chloro-6-fluoro-1-((2-(trimethylsilyl)ethoxy)methyl)-1H-benzo[d]imidazole

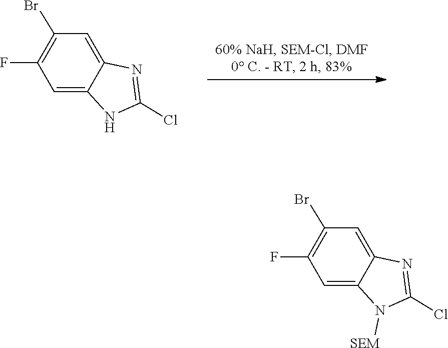 [SMILES: Fc1cc2[nH]c(Cl)nc2cc1Br ; C[Si](C)(C)CCOCn1c(Cl)nc2cc(Br)c(F)cc21]

To a stirred solution of 5-bromo-2-chloro-6-fluoro-1H-benzo[d]imidazole (1.1 g, 4.41 mmol, 1.0 eq) in DMF (10 Vol) at 0° C., was added 60% NaH (212 mg, 8.83 mmol, 2.0 eq) and stirred for 5 min. Then SEM-C₁ (0.94 mL, 5.30 mmol, 1.2 eq) was added to reaction mixture and stirred at room temperature for 2 h. After completion of reaction by TLC, the reaction mixture was quenched with ice cold water (100 mL) and extracted with EtOAc (2×100 mL). The combined organic layer was dried over Na₂SO₄, filtered and concentrated under reduced pressure to afford crude product. The crude product was purified by silica gel [100-200] column chromatography [elution with 5-10% EtOAc in petroleum ether] to afford 5-bromo-2-chloro-6-fluoro-1-((2-(trimethylsilyl)ethoxy)methyl)-1H-benzo[d]imidazole as an off-white solid (1.4 g, Yield: 83%). TLC system: EtOAc/Hexane (20:80), R_{f} value: ˜0.4; LCMS (m/z): 379.28 (M+H)⁺.

### Synthesis of 5-(3-((5-bromo-6-fluoro-1-((2-(trimethylsilyl)ethoxy)methyl)-1H-benzo[d]imidazol-2-yl)oxy)-4-methylphenyl)-1,2,5-thiadiazolidin-3-one 1,1-dioxide

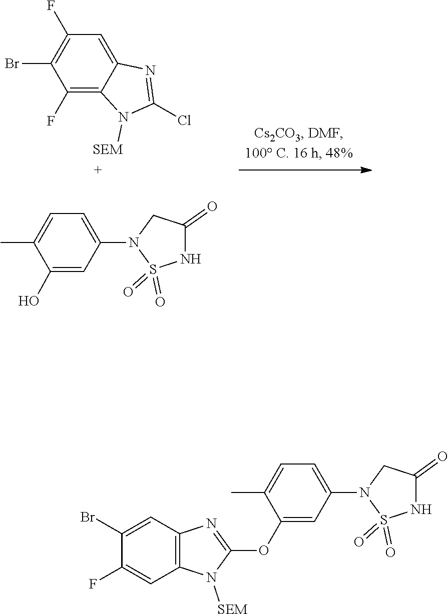 [SMILES: C[Si](C)(C)CCOCn1c(Cl)nc2cc(F)c(Br)c(F)c21 ; Cc1ccc(N2CC(=O)NS2(=O)=O)cc1O ; Cc1ccc(N2CC(=O)NS2(=O)=O)cc1Oc1nc2cc(Br)c(F)cc2n1COCC[Si](C)(C)C]

To a stirred solution of 5-bromo-2-chloro-6-fluoro-1-((2-(trimethylsilyl)ethoxy)methyl)-1H-benzo[d]imidazole (500 mg, 1.32 mmol, 1.0 eq) in DMF (10 Vol) at room temperature, was added Cs₂CO₃ (1.24 g, 3.96 mmol, 3.0 eq) followed by 5-(3-hydroxy-4-methylphenyl)-1,2,5-thiadiazolidin-3-one 1,1-dioxide (320 mg, 1.32 mmol, 1.1 eq) and stirred at 100° C. for 16 h. After completion of reaction by TLC, the reaction mixture was quenched with ice cold water (20 mL) and extracted with EtOAc (2×20 mL). The combined organic layer was dried over Na₂SO₄, filtered and concentrated under reduced pressure to afford crude product. The crude product was purified by reverse phase column chromatography [elution with 10-15% ACN in 0.1% FA in H₂O] to afford 5-(3-((5-bromo-6-fluoro-1-((2-(trimethylsilyl)ethoxy)methyl)-1H-benzo[d]imidazol-2-yl)oxy)-4-methylphenyl)-1,2,5-thiadiazolidin-3-one 1,1-dioxide as brown solid (370 mg, Yield: 48%). TLC system: MeOH/DCM (10:90), R_{f} value: ˜0.1; LCMS (m/z): 585.3 (M+H)⁺.

### Synthesis of 5-(3-((6-fluoro-5-(4-(1-hydroxycyclobutyl)phenyl)-1-((2-(trimethylsilyl)ethoxy)methyl)-1H-benzo[d]imidazol-2-yl)oxy)-4-methylphenyl)-1,2,5-thiadiazolidin-3-one 1,1-dioxide

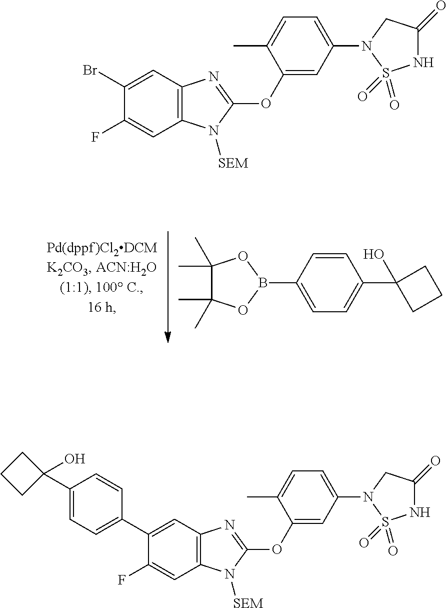 [SMILES: Cc1ccc(N2CC(=O)NS2(=O)=O)cc1Oc1nc2cc(Br)c(F)cc2n1COCC[Si](C)(C)C ; CC1(C)OB(c2ccc(C3(O)CCC3)cc2)OC1(C)C ; Cc1ccc(N2CC(=O)NS2(=O)=O)cc1Oc1nc2cc(-c3ccc(C4(O)CCC4)cc3)c(F)cc2n1COCC[Si](C)(C)C]

In a sealed tube to a degassed solution of 5-(3-((5-bromo-6-fluoro-1-((2-(trimethylsilyl)ethoxy)methyl)-1H-benzo[d]imidazol-2-yl)oxy)-4-methylphenyl)-1,2,5-thiadiazolidin-3-one 1,1-dioxide (320 mg, 0.54 mmol, 1.0 eq) and 1-(4-(4,4,5,5-tetramethyl-1,3,2-dioxaborolan-2-yl)phenyl)cyclobutan-1-ol (180 mg, 0.65 mmol, 1.2 eq) in ACN:H₂O (1:1, 10 Vol) at room temperature, was added K₂CO₃ (230 mg, 1.64 mmol, 3.0 eq) followed by Pd(dppf)Cl₂·DCM (45 mg, 0.054 mmol, 0.1 eq) and stirred at 100° C. for 16 h. After completion of reaction by TLC, the reaction mixture was diluted with water (15 mL) and extracted with EtOAc (2×15 mL). The combined organic layer was dried over Na₂SO₄, filtered and concentrated under reduced pressure to afford crude product. The crude product was triturated with diethyl ether to afford 5-(3-((6-fluoro-5-(4-(1-hydroxycyclobutyl)phenyl)-1-((2-(trimethylsilyl)ethoxy)methyl)-1H-benzo[d]imidazol-2-yl)oxy)-4-methylphenyl)-1,2,5-thiadiazolidin-3-one 1,1-dioxide as pale brown solid (400 mg, Crude). TLC system: MeOH/DCM (20:80), R_{f} value: ˜0.2; LCMS (m/z): 653.5 (M+H)⁺.

### Synthesis of 5-(3-((6-fluoro-5-(4-(1-hydroxycyclobutyl)phenyl)-1H-benzo[d]imidazol-2-yl)oxy)-4-methylphenyl)-1,2,5-thiadiazolidin-3-one 1,1-dioxide

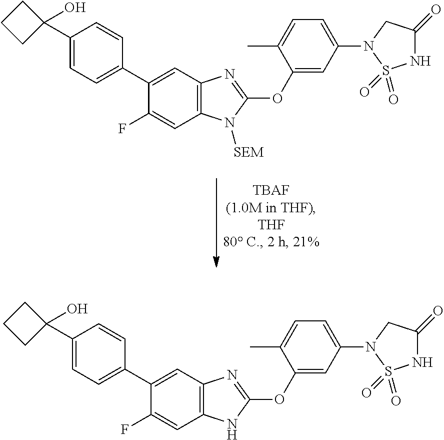 [SMILES: Cc1ccc(N2CC(=O)NS2(=O)=O)cc1Oc1nc2cc(-c3ccc(C4(O)CCC4)cc3)c(F)cc2n1COCC[Si](C)(C)C ; Cc1ccc(N2CC(=O)NS2(=O)=O)cc1Oc1nc2cc(-c3ccc(C4(O)CCC4)cc3)c(F)cc2[nH]1]

To a stirred solution of 5-(3-((6-fluoro-5-(4-(1-hydroxycyclobutyl)phenyl)-1-((2-(trimethylsilyl)ethoxy)methyl)-1H-benzo[d]imidazol-2-yl)oxy)-4-methylphenyl)-1,2,5-thiadiazolidin-3-one 1,1-dioxide (360 mg, 0.55 mmol, 1.0 eq) in THF (10 Vol) at 0° C., was added TBAF (1.0 M in THF) (10 Vol) and stirred at 80° C. for 2 h. After completion of reaction by TLC, the reaction mixture was quenched with NH₄Cl solution (20 mL) and extracted with DCM (2×50 mL). The combined organic layer was dried over Na₂SO₄, filtered and concentrated under reduced pressure. The crude product was purified by reverse phase column chromatography [elution with 35% ACN in 0.1% FA in H₂O] to afford desired product with TBAF residues. Re-purified by Prep-HPLC, collected fractions were frozen and lyophilized to afford desired product with salts. Obtained material passed through reverse phase column chromatography [elution with 30% ACN in 0.1% FA in H₂O] to afford 5-(3-((6-fluoro-5-(4-(1-hydroxycyclobutyl)phenyl)-1H-benzo[d]imidazol-2-yl)oxy)-4-methylphenyl)-1,2,5-thiadiazolidin-3-one 1,1-dioxide as a white solid (15 mg, Yield: 21%). TLC system: MeOH/DCM (20:80), R_{f} value: ˜0.1; LCMS (m/z): 523.42 (M+H)⁺; ¹H NMR (400 MHz, DMSO-d₆) δ: 7.57-7.55 (m, 2H), 7.50 (dd, J=1.6 Hz, 8.4 Hz, 2H), 7.37 (d, J=7.2 Hz, 1H), 7.29-7.24 (m, 2H), 6.99-6.95 (m, 2H), 4.01 (s, 2H), 2.50-2.40 (m, 2H), 2.33-2.25 (m, 2H), 2.09 (s, 3H), 1.97-1.87 (m, 1H), 1.68-1.65 (m, 1H).

Prep-HPLC condition: Column YMC C-18 (250*21.2 mm) 5μ Buffer MP-(A): 10 Mm ABC in H2O MP-(B): ACN:WATER (80:20) Gradient (T/% B) 0/30, 10/65, 25/98 Flow Rate 15 ml/min Solubility ACN, WATER, THF

### SYNTHETIC EXAMPLE 66

### Synthesis of Compound II-23

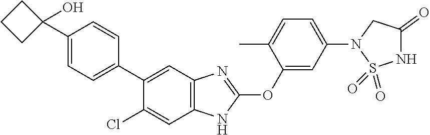 [SMILES: Cc1ccc(N2CC(=O)NS2(=O)=O)cc1Oc1nc2cc(-c3ccc(C4(O)CCC4)cc3)c(Cl)cc2[nH]1]

### Synthesis of 5-bromo-6-chloro-1,3-dihydro-2H-benzo[d]imidazol-2-one

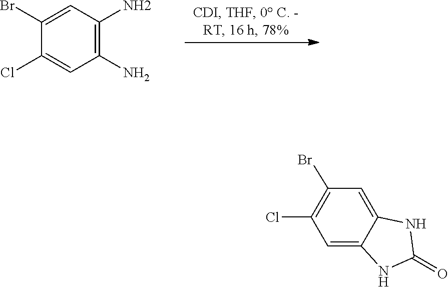 [SMILES: Nc1cc(Cl)c(Br)cc1N ; O=c1[nH]c2cc(Cl)c(Br)cc2[nH]1]

To a stirred solution of 4-bromo-5-chlorobenzene-1,2-diamine (5 g, 22.62 mmol, 1.0 eq) in THF (10 Vol) at 0° C., was added CDI (4.4 g, 27.15 mmol, 1.2 eq) and stirred at room temperature for 16 h. After completion of reaction by TLC, the reaction mixture was quenched with water (50 mL), solid was filtered, washed with H₂O (60 mL) and collected solid was dried to afford crude. The crude product was triturated with diethyl ether (50 mL), decanted and dried under reduced pressure to afford 5-bromo-6-chloro-1,3-dihydro-2H-benzo[d]imidazol-2-one as pale brown solid (4.3 g, Yield: 78%). TLC system: EtOAc/Hexane (100:00), R_{f} value: ˜0.2; ¹H NMR (400 MHz, DMSO-d₆) δ: 10.92-10.90 (s, 2H), 7.21 (s, 2H), 7.12 (s, 1H).

### Synthesis of 5-bromo-2,6-dichloro-1H-benzo[d]imidazole

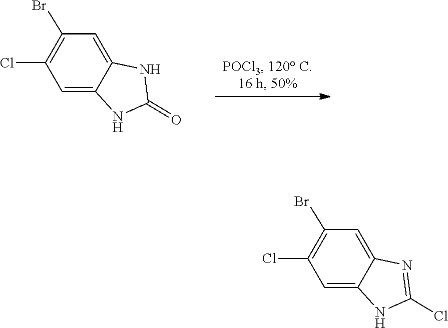 [SMILES: O=c1[nH]c2cc(Cl)c(Br)cc2[nH]1 ; Clc1nc2cc(Br)c(Cl)cc2[nH]1]

A solution of 5-bromo-6-chloro-1,3-dihydro-2H-benzo[d]imidazol-2-one (2.3 g, 9.31 mmol, 1.0 eq) in POCl₃ (6.9 mL, 3 Vol) was stirred at 120° C. for 16 h. After completion of reaction by TLC, the reaction mixture was quenched with NaHCO₃ (50 mL), diluted with H₂O (50 mL) and extracted with EtOAc (2×50 mL). The combined organic layer was dried over Na₂SO₄, filtered and concentrated under reduced pressure to afford crude product. The crude product was purified by silica gel [100-200] column chromatography [elution with 30% EtOAc in petroleum ether] to afford 5-bromo-2,6-dichloro-1H-benzo[d]imidazole as a pale brown solid (1.2 g, Yield: 50%). TLC system: EtOAc/Hexane (30:70), R_{f} value: ˜0.4; LCMS (m/z): 265.12 (M+H)⁺.

### Synthesis of 5-bromo-2,6-dichloro-1-((2-(trimethylsilyl)ethoxy)methyl)-1H-benzo[d]imidazole

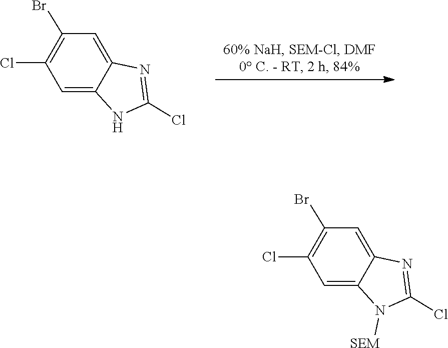 [SMILES: Clc1nc2cc(Br)c(Cl)cc2[nH]1 ; C[Si](C)(C)CCOCn1c(Cl)nc2cc(Br)c(Cl)cc21]

To a stirred solution of 5-bromo-2,6-dichloro-1H-benzo[d]imidazole (1.2 g, 4.54 mmol, 1.0 eq) in DMF (10 Vol) at 0° C., was added 60% NaH (218 mg, 9.09 mmol, 2.0 eq) and stirred for 5 min. Then SEM-Cl (1.0 mL, 5.45 mmol, 1.2 eq) was added to reaction mixture and stirred at room temperature for 2 h. After completion of reaction by TLC, the reaction mixture was quenched with ice cold water (50 mL) and extracted with EtOAc (2×100 mL). The combined organic layer was dried over Na₂SO₄, filtered and concentrated under reduced pressure to afford crude product. The crude product was purified by silica gel [100-200] column chromatography [elution with 5-10% EtOAc in petroleum ether] to afford 5-bromo-2,6-dichloro-1-((2-(trimethylsilyl)ethoxy)methyl)-1H-benzo[d]imidazole as a pale brown solid (1.5 g, Yield: 84%). TLC system: EtOAc/Hexane (20:80), R_{f} value: ˜0.5; LCMS (m/z): 395.21 (M+H)⁺; mixture of regio-isomers observed by NMR

### Synthesis of 5-(3-((5-bromo-6-chloro-1-((2-(trimethylsilyl)ethoxy)methyl)-1H-benzo[d]imidazol-2-yl)oxy)-4-methylphenyl)-1,2,5-thiadiazolidin-3-one 1,1-dioxide

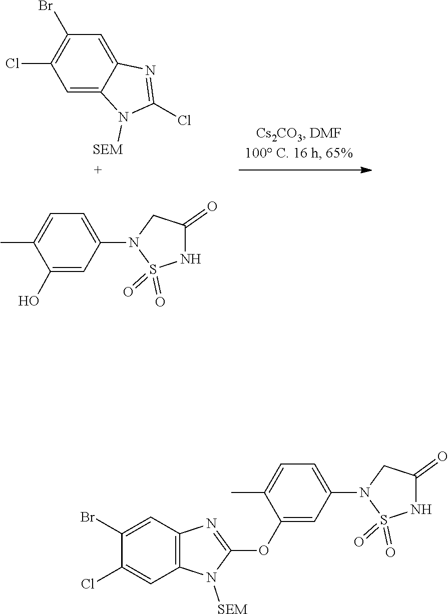 [SMILES: C[Si](C)(C)CCOCn1c(Cl)nc2cc(Br)c(Cl)cc21 ; Cc1ccc(N2CC(=O)NS2(=O)=O)cc1O ; Cc1ccc(N2CC(=O)NS2(=O)=O)cc1Oc1nc2cc(Br)c(Cl)cc2n1COCC[Si](C)(C)C]

To a stirred solution of 5-bromo-2,6-dichloro-1-((2-(trimethylsilyl)ethoxy)methyl)-1H-benzo[d]imidazole (500 mg, 1.27 mmol, 1.0 eq) in DMF (10 Vol) at room temperature, was added Cs₂CO₃ (1.24 g, 3.81 mmol, 3.0 eq) followed by 5-(3-hydroxy-4-methylphenyl)-1,2,5-thiadiazolidin-3-one 1,1-dioxide (338 mg, 1.39 mmol, 1.1 eq) and stirred at 100° C. for 16 h. After completion of reaction by TLC, the reaction mixture was quenched with ice cold water (20 mL) and extracted with EtOAc (2×20 mL). The combined organic layer was dried over Na₂SO₄, filtered and concentrated under reduced pressure to afford crude product. The crude product was purified by reverse phase column chromatography [elution with 45% ACN in 0.1% FA in H₂O] to afford 5-(3-((5-bromo-6-chloro-1-((2-(trimethylsilyl)ethoxy)methyl)-1H-benzo[d]imidazol-2-yl)oxy)-4-methylphenyl)-1,2,5-thiadiazolidin-3-one 1,1-dioxide as a brown solid (550 mg, Yield: 65%). TLC system: MeOH/DCM (10:90), R_{f} value: ˜0.5; LCMS (m/z): 601.22 (M+H)⁺.

### Synthesis of 5-(3-((6-chloro-5-(4-(1-hydroxycyclobutyl)phenyl)-1-((2-(trimethylsilyl)ethoxy)methyl)-1H-benzo[d]imidazol-2-yl)oxy)-4-methylphenyl)-1,2,5-thiadiazolidin-3-one 1,1-dioxide

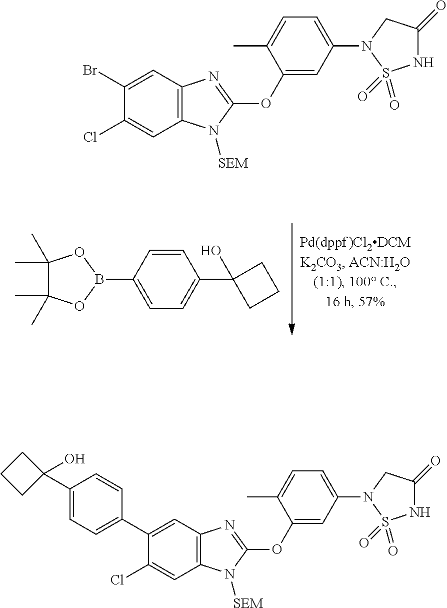 [SMILES: Cc1ccc(N2CC(=O)NS2(=O)=O)cc1Oc1nc2cc(Br)c(Cl)cc2n1COCC[Si](C)(C)C ; CC1(C)OB(c2ccc(C3(O)CCC3)cc2)OC1(C)C ; Cc1ccc(N2CC(=O)NS2(=O)=O)cc1Oc1nc2cc(-c3ccc(C4(O)CCC4)cc3)c(Cl)cc2n1COCC[Si](C)(C)C]

In a sealed tube, to a degassed solution of 5-(3-((5-bromo-6-chloro-1-((2-(trimethylsilyl)ethoxy)methyl)-1H-benzo[d]imidazol-2-yl)oxy)-4-methylphenyl)-1,2,5-thiadiazolidin-3-one 1,1-dioxide (500 mg, 0.83 mmol, 1.0 eq) and 1-(4-(4,4,5,5-tetramethyl-1,3,2-dioxaborolan-2-yl)phenyl)cyclobutan-1-ol (274 mg, 0.99 mmol, 1.2 eq) in ACN:H₂O (1:1, 10 Vol) at room temperature was added K₂CO₃ (344 mg, 2.49 mmol, 3.0 eq) followed by Pd(dppf)Cl₂·DCM (68 mg, 0.083 mmol, 0.1 eq) and stirred at 100° C. for 16 h. After completion of reaction by TLC, the reaction mixture was diluted with water (15 mL) and extracted with EtOAc (2× 15 mL). The combined organic layer was dried over Na₂SO₄, filtered and concentrated under reduced pressure to afford crude product. The crude product was purified by reverse phase column chromatography [elution with 35% ACN in 0.1% FA in H₂O] to afford 5-(3-((6-chloro-5-(4-(1-hydroxycyclobutyl)phenyl)-1-((2-(trimethylsilyl)ethoxy)methyl)-1H-benzo[d]imidazol-2-yl)oxy)-4-methylphenyl)-1,2,5-thiadiazolidin-3-one 1,1-dioxide as brown solid (350 mg, Yield: 57%). TLC system: MeOH/DCM (20:80), R_{f} value: ˜0.4; LCMS (m/z): 669.5 (M+H)⁺.

### Synthesis of 5-(3-((6-chloro-5-(4-(1-hydroxycyclobutyl)phenyl)-1H-benzo[d]imidazol-2-yl)oxy)-4-methylphenyl)-1,2,5-thiadiazolidin-3-one 1,1-dioxide

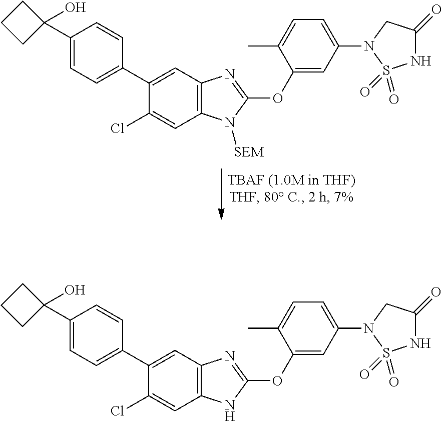 [SMILES: Cc1ccc(N2CC(=O)NS2(=O)=O)cc1Oc1nc2cc(-c3ccc(C4(O)CCC4)cc3)c(Cl)cc2n1COCC[Si](C)(C)C ; Cc1ccc(N2CC(=O)NS2(=O)=O)cc1Oc1nc2cc(-c3ccc(C4(O)CCC4)cc3)c(Cl)cc2[nH]1]

To a stirred solution of 5-(3-((6-chloro-5-(4-(1-hydroxycyclobutyl)phenyl)-1-((2-(trimethylsilyl)ethoxy)methyl)-1H-benzo[d]imidazol-2-yl)oxy)-4-methylphenyl)-1,2,5-thiadiazolidin-3-one 1,1-dioxide (340 mg, 0.51 mmol, 1.0 eq) in THF (10 Vol) at 0° C., was added TBAF (1.0 M in THF) (10 Vol) and stirred at 80° C. for 2 h. After completion of reaction by TLC, reaction mixture was quenched with NH₄Cl solution (20 mL) and extracted with DCM (2×20 mL). The combined organic layer was dried over Na₂SO₄, filtered and concentrated under reduced pressure. The crude product was purified by reverse phase column chromatography [elution with 35% ACN in 0.1% FA in H₂O] followed by prep-HPLC and lyophilized pure fractions to afford 5-(3-((6-chloro-5-(4-(1-hydroxycyclobutyl)phenyl)-1H-benzo[d]imidazol-2-yl)oxy)-4-methylphenyl)-1,2,5-thiadiazolidin-3-one 1,1-dioxide as white solid (19 mg, Yield: 7%). TLC system: MeOH/DCM (20:80), R_{f} value: ˜0.1; LCMS (m/z): 539.39 (M+H)⁺; ¹H NMR (400 MHz, DMSO-d₆) δ: 7.54 (d, J=8.4 Hz, 2H), 7.50 (s, 1H), 7.39 (d, J=8.4 Hz, 2H), 7.30 (s, 1H), 7.25 (d, J=8.4 Hz, 1H), 7.00-6.96 (m, 2H), 4.02 (s, 2H), 2.45-2.41 (m, 2H), 2.32-2.25 (m, 2H), 2.09 (s, 3H), 1.96-1.91 (m, 1H), 1.71-1.67 (m, 1H).

Prep-HPLC condition: Column X-Bridge C-18 (250·19.0 mm) 5μ Buffer MP-(A): 10 Mm Ammonium acetate IN WATER MP-(B): ACN Gradient (T/% B) 0/5, 13/58, 16/58, 25/95 Flow Rate 15 mL/min Solubility acetonitrile and ATER.

### SYNTHETIC EXAMPLE 67

### Synthesis of Compound III-3

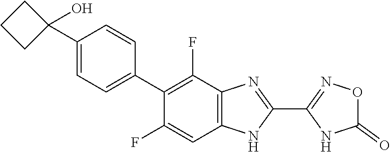 [SMILES: O=c1[nH]c(-c2nc3c(F)c(-c4ccc(C5(O)CCC5)cc4)c(F)cc3[nH]2)no1]

### Synthesis of N-(4-bromo-3,5-difluoro-2-nitrophenyl)acetamide

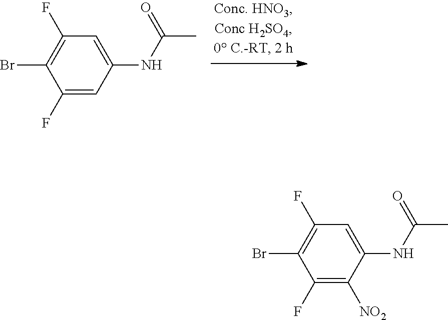 [SMILES: CC(=O)Nc1cc(F)c(Br)c(F)c1 ; CC(=O)Nc1cc(F)c(Br)c(F)c1[N+](=O)[O-]]

To a stirred solution of N-(4-bromo-3,5-difluorophenyl)acetamide (25 g, 100.4 mol, 1.0 eq) in concentrated HNO₃ (35 mL, 1.4 vol) at 0° C., was added concentrated H₂SO₄ (35 mL, 1.4 vol) dropwise. After 10 min, the reaction mixture was allowed to room temperature, stirred for 2 h. After completion of reaction by TLC, diluted with chilled H₂O (250 mL, 10 vol) and stirred for 30 min at RT. Solid was filtered and washed with water (100 ml, 2 vol), dried to afford N-(4-bromo-3,5-difluoro-2-nitrophenyl)acetamide as light-yellow color solid (18 g, crude). TLC system: EtOAc/Hexane (20:80), R_{f} value: ˜0.3; LCMS (m/z): 251.0 (M−Ac)⁺.

### Synthesis of 4-bromo-3,5-difluoro-2-nitroaniline

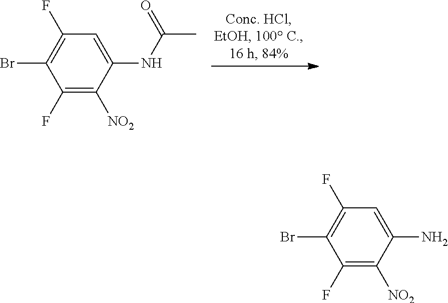 [SMILES: CC(=O)Nc1cc(F)c(Br)c(F)c1[N+](=O)[O-] ; Nc1cc(F)c(Br)c(F)c1[N+](=O)[O-]]

To a stirred of solution of N-(4-bromo-3,5-difluoro-2-nitrophenyl)acetamide (18 g, 61.23 mmol, 1.0 eq) in EtOH (180 mL, 10 vol) at 0° C., was added Conc. HCl (90 mL, 5 vol) slowly, and reaction mixture was heated at 100° C. for 16 h. After completion of reaction by TLC, the reaction mixture was concentered under reduced pressure. the reaction mixture was diluted with water (150 mL) and extracted with EtOAc (2×200 mL). Organic layer was washed with aq. NaHCO₃ solution (100 mL), dried over Na₂SO₄, filtered and concentrated under reduced pressure to afford 4-bromo-3,5-difluoro-2-nitroaniline as a yellow color solid (13 g, Yield: 84%). TLC system: EtOAc/Hexane (20:80), R_{f} value: ˜0.2; LCMS (m/z): 250.94 (M−H)⁺; ¹H NMR (400 MHz, CDCl₃) δ: 6.44 (dd, J=7.6 Hz, 2.0 Hz, 1H), 5.86 (brs, 2H).

### Synthesis of 4-bromo-3,5-difluorobenzene-1,2-diamine

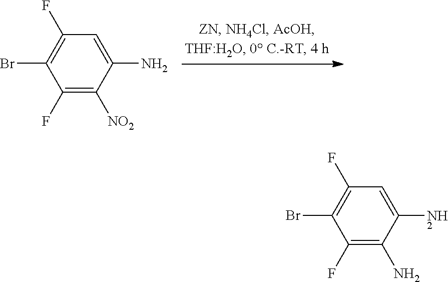 [SMILES: Nc1cc(F)c(Br)c(F)c1[N+](=O)[O-] ; Nc1cc(F)c(Br)c(F)c1N]

To a stirred solution of 4-bromo-3,5-difluoro-2-nitroaniline (13 g, 51.6 mmol, 1.0 eq) in THF:H₂O (1:1) (260 mL, 20 vol) at 0° C., was added NH₄Cl (11.04 g, 206.4 mmol, 4 eq), Zn dust (13.49 g, 206.4 mmol, 4 eq) followed by acetic acid (3.9 mL, 0.3 vol), and stirred at room temperature for 4 h. After completion of reaction by TLC, the reaction mixture was filtered through a pad of diatomaceous earth (e.g., Celite®), and washed with 10% MeOH/DCM (200 mL). Filtrate was concentrated under reduced pressure to afford 4-bromo-3,5-difluorobenzene-1,2-diamine as a brown color solid (8 g, Yield: 70%). TLC system: EtOAc/Hexane (50:50), R_{f} value: ˜0.1; LCMS (m/z): 223.1 (M+H)⁺.

### Synthesis of 5-bromo-4,6-difluoro-2-(trichloromethyl)-1H-benzo[d]imidazole

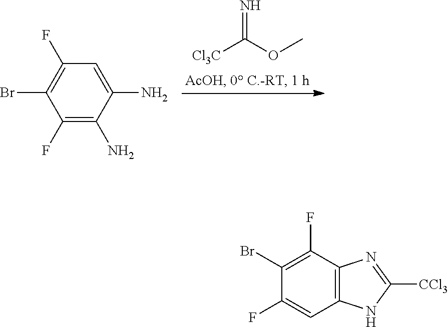

To a stirred solution of 4-bromo-3,5-difluorobenzene-1,2-diamine (4 g, 18.02 mmol, 1.0 eq) in acetic acid (160 mL, 40 vol) at 0° C., was added methyl 2,2,2-trichloroacetimidate (6.30 g, 36.04 mmol, 2.0 eq), and stirred at room temperature for 1 h. After completion of reaction by TLC, the reaction mixture was diluted with water (60 mL) and extracted with EtOAc (2×150 mL). The combined organic layer was dried over Na₂SO₄, filtered and concentrated under reduced pressure to afford 5-bromo-4,6-difluoro-2-(trichloromethyl)-1H-benzo[d]imidazole as a black color solid (5 g, crude). TLC system: EtOAc/Hexane (20:20), R_{f} value: ˜0.3. Material was used as such in next step immediately.

### Synthesis of 5-bromo-4,6-difluoro-1H-benzo[d]imidazole-2-carboxylic acid

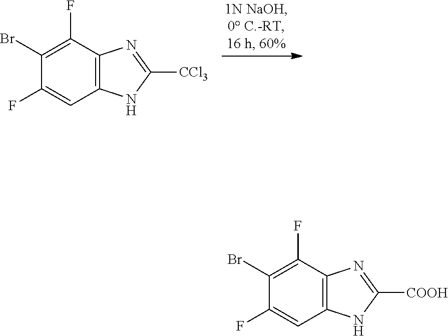

A solution of 5-bromo-4,6-difluoro-2-(trichloromethyl)-1H-benzo[d]imidazole (5 g, 14.37 mmol, 1.0 eq) in 1N aq. NaOH (50 mL, 10 vol) was allowed to stir at room temperature for 16 h. After completion of reaction by TLC, the reaction mixture was acidified with 1N aq. HCl (200 mL) and extracted with EtOAc (2×200 mL). The combined organic layer was dried over Na₂SO₄, filtered and concentrated under reduced pressure. Obtained material was triturated with n-pentane and dried to afford 5-bromo-4,6-difluoro-1H-benzo[d]imidazole-2-carboxylic acid as black color solid (3 g, Yield: 60% in 2 steps). TLC system: EtOAc/Hexane (50:50), R_{f} value: ^{˜}0.1; LCMS (m/z): 277.06 (M+H)⁺.

### Synthesis of 5-bromo-4,6-difluoro-1H-benzo[d]imidazole-2-carboxamide

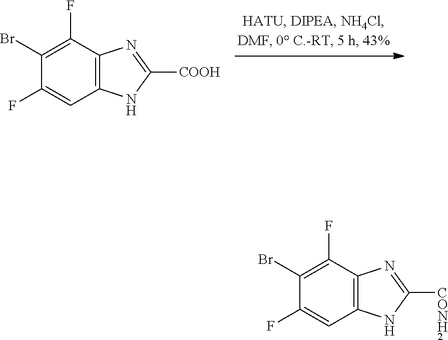 [SMILES: O=C(O)c1nc2c(F)c(Br)c(F)cc2[nH]1 ; NC(=O)c1nc2c(F)c(Br)c(F)cc2[nH]1]

To a stirred solution of 5-bromo-4,6-difluoro-1H-benzo[d]imidazole-2-carboxylic acid (3 g, 10.87 mmol, 1 eq) in DMF (30 mL, 10 vol) at 0° C., was added HATU (4.96 g, 13.04 mmol, 1.2 eq), DIPEA (2.8 g, 21.74 mmol, 2 eq) and stirred for 15 min. Later, added NH₄Cl (872.1 mg, 16.3 mmol, 1.5 eq) and stirred at room temperature for 5 h. After completion of reaction by TLC, diluted with chilled H₂O (165 mL, 5 vol) and stirred for 30 min at RT. Precipitated solid was filtered, washed with water & pentane and dried to afford 5-bromo-4,6-difluoro-1H-benzo[d]imidazole-2-carboxamide as a yellow solid (1.3 g Yield: 43%), TLC system: EtOAc/Hexane (50:50%), R_{f} value: ˜0.2; LCMS (m/z): 274.0 (M−H)⁺.

### Synthesis of 5-bromo-4,6-difluoro-1H-benzo[d]imidazole-2-carbonitrile

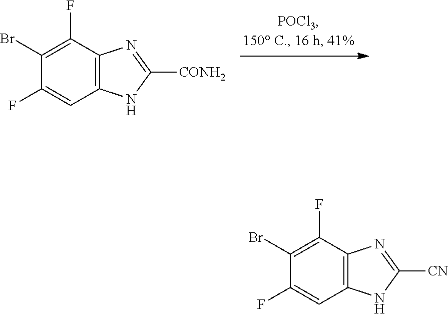 [SMILES: NC(=O)c1nc2c(F)c(Br)c(F)cc2[nH]1 ; N#Cc1nc2c(F)c(Br)c(F)cc2[nH]1]

A solution of 5-bromo-4,6-difluoro-1H-benzo[d]imidazole-2-carboxamide (1.3 g, 4.72 mmol, 1.0 eq) in POCl₃ (45.5 mL, 35 vol) was heated to 150° C., stirred for 16 h. After completion of reaction by TLC, reaction mixture was cooled to room temperature, concentrated under reduced pressure to afford crude compound. The crude compound was purified by silica column (100-200 mesh) [elution with 50% EtOAc in Hexane] to afford 5-bromo-4,6-difluoro-1H-benzo[d]imidazole-2-carbonitrile as a colorless liquid (500 mg, Yield: 41%). TLC system: EtOAc/Hexane (50:50%), R_{f} value: ˜0.3; LCMS (m/z): 256.04 (M−H)⁻.

### Synthesis of 5-bromo-4,6-difluoro-1-((2-(trimethylsilyl)ethoxy)methyl)-1H-benzo[d]imidazole-2-carbonitrile

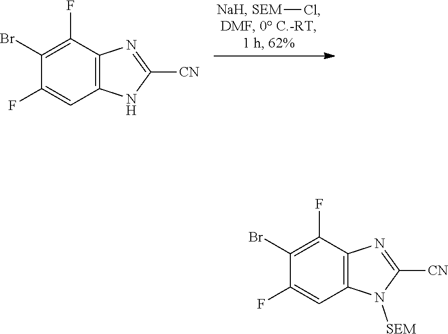

To a stirred solution of 5-bromo-4,6-difluoro-1H-benzo[d]imidazole-2-carbonitrile (500 mg, 1.94 mmol, 1.0 eq) in DMF (5 mL, 10 vol) at 0° C., was added 100% NaH (145.87 mg, 3.89 mmol, 2.0 eq) and stirred for 30 min. Later, added SEM-C₁ (0.41 mL, 2.33 mmol, 1.2 eq) and reaction mixture was slowly warm-up at room temperature and stirred for another 1 h. After completion of reaction by TLC, the reaction mixture was diluted with ice cold water (30 mL) and extracted with EtOAc (2×100 mL). The combined organic layer was dried over Na₂SO₄, filtered and concentrated under reduced pressure. The crude product was purified by silica gel (100-200 mesh) column chromatography [elution with 20% EtOAc in Hexane] to afford 5-bromo-4,6-difluoro-1-((2-(trimethylsilyl)ethoxy)methyl)-1H-benzo[d]imidazole-2-carbonitrile as colorless liquid (470 mg, Yield: 62%). TLC system: EtOAc/Hexane (20:80), R_{f} value: ˜0.3; LCMS (m/z): No proper ionization

### Synthesis of (Z)-5-bromo-4,6-difluoro-N′-hydroxy-1-((2-(trimethylsilyl)ethoxy)methyl)-1H-benzo[d]imidazole-2-carboximidamide

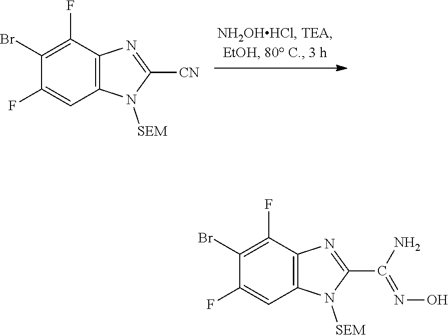 [SMILES: C[Si](C)(C)CCOCn1c(C#N)nc2c(F)c(Br)c(F)cc21 ; C[Si](C)(C)CCOCn1c(/C(N)=N/O)nc2c(F)c(Br)c(F)cc21]

To a stirred solution of 5-bromo-4,6-difluoro-1-((2-(trimethylsilyl)ethoxy)methyl)-1H-benzo[d]imidazole-2-carbonitrile (470 mg, 1.21 mmol, 1.0 eq) was added TEA (0.5 mL, 3.64 mmol, 3.0 eq), NH₂OH·HCl (168 mg, 2.42 mmol, 2.0 eq) and stirred at 80° C. for 3 h. After completion of reaction by TLC, reaction mixture was concentrated under reduced pressure, diluted with ice cold water (50 mL) and extracted with EtOAc (2×150 mL). The combined organic layer was dried over Na₂SO₄, filtered and concentrated under reduced pressure to afford (Z)-5-bromo-4,6-difluoro-N′-hydroxy-1-((2-(trimethylsilyl)ethoxy)methyl)-1H-benzo[d]imidazole-2-carboximidamide a colorless gummy liquid (400 mg, Crude). TLC system: EtOAc/Hexane (30:70), R_{f} value: ˜0.2; LCMS (m/z): 419.18 (M−H)⁻.

### Synthesis of 3-(5-bromo-4,6-difluoro-1-((2-(trimethylsilyl)ethoxy)methyl)-1H-benzo[d]imidazol-2-yl)-1,2,4-oxadiazol-5(4H)-one

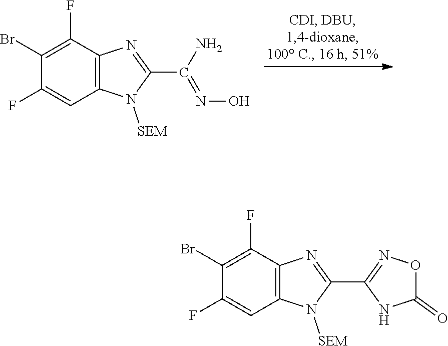 [SMILES: C[Si](C)(C)CCOCn1c(/C(N)=N/O)nc2c(F)c(Br)c(F)cc21 ; C[Si](C)(C)CCOCn1c(-c2noc(=O)[nH]2)nc2c(F)c(Br)c(F)cc21]

To a stirred of solution of Z)-5-bromo-4,6-difluoro-N′-hydroxy-1-((2-(trimethylsilyl)ethoxy)methyl)-1H-benzo[d]imidazole-2-carboximidamide (400 mg, 0.95 mmol, 1.0 eq) in 1,4-dioxane (4 mL, 10 vol), was added DBU (289.9 mg, 1.90 mmol, 2.0 eq) CDI (308.1 mg, 1.90 mmol, 2.0 eq) at room temperature and reaction mixture was heated to 100° C. for 16 h. After completion of reaction by TLC, reaction mixture was diluted with ice cold water (300 mL) and acidified with 2N HCl, stirred at room temperature for 20 min. The reaction mixture was filtered, solid was washed with pentane (10 mL), and dried to afford 3-(5-bromo-4,6-difluoro-1-((2-(trimethylsilyl)ethoxy)methyl)-1H-benzo[d]imidazol-2-yl)-1,2,4-oxadiazol-5(4H)-one as colorless gummy solid (215 mg, Yield: 51%). TLC system: EtOAc/Hexane (50:50), R_{f} value: ˜0.2; LCMS (m/z): 447.17 (M+H)⁺.

### Synthesis of 3-(4,6-difluoro-5-(4-(1-hydroxycyclobutyl)phenyl)-1-((2-(trimethylsilyl)ethoxy)methyl)-1H-benzo[d]imidazol-2-yl)-1,2,4-oxadiazol-5(4H)-one

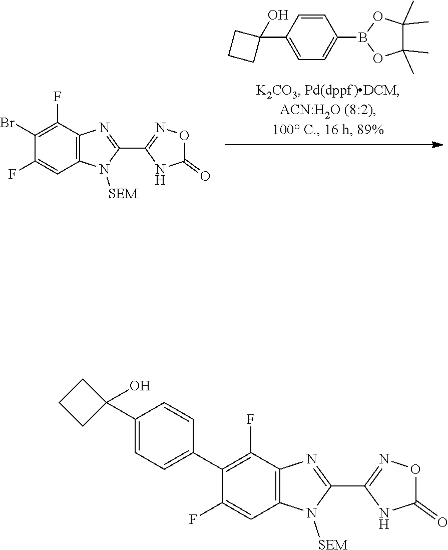 [SMILES: CC1(C)OB(c2ccc(C3(O)CCC3)cc2)OC1(C)C ; C[Si](C)(C)CCOCn1c(-c2noc(=O)[nH]2)nc2c(F)c(Br)c(F)cc21 ; C[Si](C)(C)CCOCn1c(-c2noc(=O)[nH]2)nc2c(F)c(-c3ccc(C4(O)CCC4)cc3)c(F)cc21]

In a sealed tube to a degassed solution of 3-(5-bromo-4,6-difluoro-1-((2-(trimethylsilyl)ethoxy)methyl)-1H-benzo[d]imidazol-2-yl)-1,2,4-oxadiazol-5(4H)-one (215 mg, 0.48 mmol, 1.0 eq) and 1-(4-(4,4,5,5-tetramethyl-1,3,2-dioxaborolan-2-yl)phenyl)cyclobutan-1-ol (198.2 mg, 0.72 mmol, 1.5 eq) in ACN:H₂O (8:2) (4.3 mL, 20 vol) at room temperature, was added K₂CO₃ (198.7 mg, 1.44 mmol, 3.0 eq) followed by PdCl₂(dppf)·DCM (39.2 mg, 0.048 mmol, 0.1 eq) and stirred at 100° C. for 16 h. After completion of reaction by TLC, the reaction mixture was diluted with water (30 mL) and extracted with EtOAc (2×80 mL). The combined organic layer was dried over Na₂SO₄, filtered and concentrated under reduced pressure. Obtained crude was purified by reverse phase C18 column chromatography [eluted with ˜45% ACN and 0.1% FA in H₂O], collected fractions were evaporated to under reduced pressure to afford 3-(4,6-difluoro-5-(4-(1-hydroxycyclobutyl)phenyl)-1-((2-(trimethylsilyl)ethoxy)methyl)-1H-benzo[d]imidazol-2-yl)-1,2,4-oxadiazol-5(4H)-one as a colorless gummy solid (220 mg, Yield: 89%). TLC system: EtOAc/Hexane (50:50), R_{f} value: ˜0.15; LCMS (m/z): 515.34 (M+H)⁺.

### Synthesis of 3-(4,6-difluoro-5-(4-(1-hydroxycyclobutyl)phenyl)-1H-benzo[d]imidazol-2-yl)-1,2,4-oxadiazol-5(4H)-one

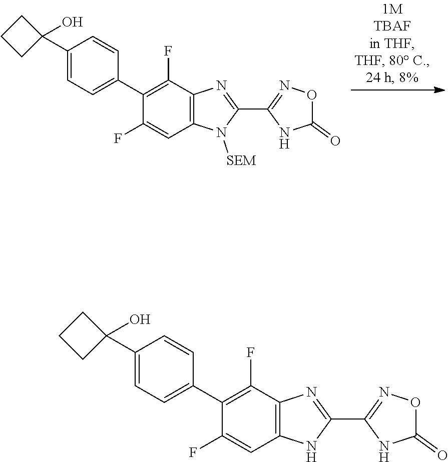 [SMILES: C[Si](C)(C)CCOCn1c(-c2noc(=O)[nH]2)nc2c(F)c(-c3ccc(C4(O)CCC4)cc3)c(F)cc21 ; O=c1[nH]c(-c2nc3c(F)c(-c4ccc(C5(O)CCC5)cc4)c(F)cc3[nH]2)no1]

To a stirred solution of 3-(4,6-difluoro-5-(4-(1-hydroxycyclobutyl)phenyl)-1-((2-(trimethylsilyl)ethoxy)methyl)-1H-benzo[d]imidazol-2-yl)-1,2,4-oxadiazol-5(4H)-one (220 mg, 0.43 mmol, 1.0 eq) in THF (2.2 mL, 10 vol) at 0° C., was added TBAF (4.4 mL, 20 vol) and stirred at 80° C. for 24 h. After completion of reaction by TLC, the reaction mixture was quenched with aq. NH₄Cl solution (30 mL) and extracted with EtOAc (2×80 mL). The combined organic layer was dried over Na₂SO₄, filtered and concentrated under reduced pressure. Obtained crude was purified by Prep-HPLC, collected fractions were lyophilized to afford 3-(4,6-difluoro-5-(4-(1-hydroxycyclobutyl)phenyl)-1H-benzo[d]imidazol-2-yl)-1,2,4-oxadiazol-5(4H)-one as off white solid (16 mg, Yield: 8%). TLC system: EtOAc (100%), R_{f} value: ˜0.1; LCMS (m/z): 383.14 (M−H)⁻; ¹H NMR (400 MHz, DMSO-d₆) δ: 14.23-13.88 (m, 2H), 7.62 (d, J=8.4 Hz, 2H), 7.46 (d, J=8.0 Hz, 2H), 7.41-7.39 (m, 1H), 5.57 (brs, 1H), 2.49-2.41 (m, 2H), 2.35-2.27 (m, 2H), 2.00-1.97 (m, 1H), 1.75-1.70 (m, 1H).

Prep-HPLC condition: Gemini NX C-18 (25*21.2) mm 5mic, Buffer: MP-(A): 0.1% FA in Water MP-(B): ACN; Gradient (T/% B) 0/45, 12/65, 20/95, 25/98; Flow Rate: 13 ml/min; Solubility: ACN/THF/water

### SYNTHETIC EXAMPLE 68

### Synthesis of Compound II-25

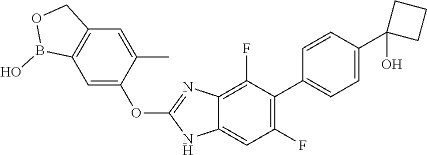 [SMILES: Cc1cc2c(cc1Oc1nc3c(F)c(-c4ccc(C5(O)CCC5)cc4)c(F)cc3[nH]1)B(O)OC2]

### Synthesis of 6-((4,6-difluoro-5-(4-(1-hydroxycyclobutyl)phenyl)-1-((2-(trimethylsilyl)ethoxy)methyl)-1H-benzo[d]imidazol-2-yl)oxy)-5-methylbenzo[c][1,2]oxaborol-1(3H)-ol

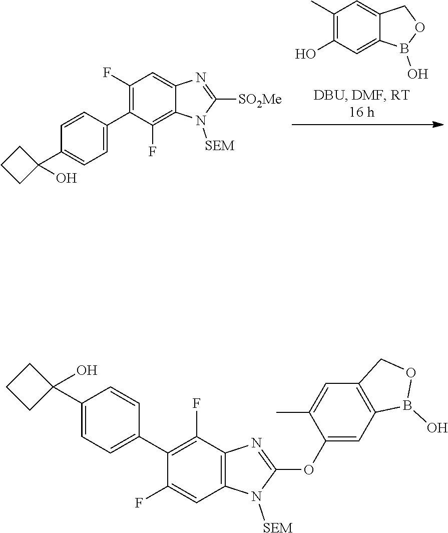

To a stirred solution of 1-(4-(5,7-difluoro-2-(methylsulfonyl)-1-((2-(trimethylsilyl)ethoxy)methyl)-1H-benzo[d]imidazol-6-yl)phenyl)cyclobutan-1-ol (160 mg, 0.31 mmol, 1.0 eq) in DMF (10 Vol) at room temperature, was added DBU (141 mg, 0.93 mmol, 3.0 eq) followed by 5-methylbenzo[c][1,2]oxaborole-1,6 (3H)-diol (80 mg, 0.47 mmol, 1.5 eq) and stirred at room temperature for 16 h. After completion of reaction by TLC, the reaction mixture was diluted with water (10 mL) and extracted with EtOAc (2×15 mL). The combined organic layer was concentrated under reduced pressure to afford crude product. The crude product was purified by silica gel [100-200] column chromatography [elution with EtOAc to 10% MeOH in DCM] to afford 6-((4,6-difluoro-5-(4-(1-hydroxycyclobutyl)phenyl)-1-((2-(trimethylsilyl)ethoxy)methyl)-1H-benzo[d]imidazol-2-yl)oxy)-5-methylbenzo[c][1,2]oxaborol-1(3H)-ol as a yellow gum (190 mg). TLC system: EtOAc/Hexane (50:50), R_{f} value: ˜0.2; LCMS (m/z): 593.57 (M+H)⁺.

### Synthesis of 6-((4,6-difluoro-5-(4-(1-hydroxycyclobutyl)phenyl)-1H-benzo[d]imidazol-2-yl)oxy)-5-methylbenzo[c][1,2]oxaborol-1(3H)-ol

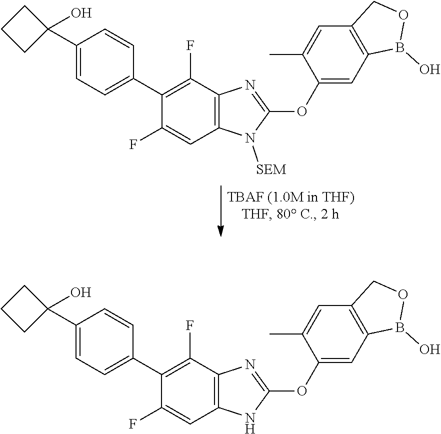 [SMILES: Cc1cc2c(cc1Oc1nc3c(F)c(-c4ccc(C5(O)CCC5)cc4)c(F)cc3n1COCC[Si](C)(C)C)B(O)OC2 ; Cc1cc2c(cc1Oc1nc3c(F)c(-c4ccc(C5(O)CCC5)cc4)c(F)cc3[nH]1)B(O)OC2]

To a stirred solution of 6-((4,6-difluoro-5-(4-(1-hydroxycyclobutyl)phenyl)-1-((2-(trimethylsilyl)ethoxy)methyl)-1H-benzo[d]imidazol-2-yl)oxy)-5-methylbenzo[c][1,2]oxaborol-1(3H)-ol (190 mg, 0.32 mmol, 1.0 eq) in THF (10 Vol) at 0° C., was added TBAF (1.0 M in THF) (10 Vol) and stirred at 80° C. for 2 h. After completion of reaction by TLC, the reaction mixture was quenched with NH₄Cl solution (10 mL) and extracted with DCM (2× 10 mL). The combined organic layer was dried over Na₂SO₄, filtered and concentrated under reduced pressure to afford crude product. The crude product was triturated with n-pentane (15 mL), diethyl ether (5 mL), decanted and dried under reduced pressure to afford crude product. Obtained crude purified by Prep-HPLC, collected fractions were frozen and lyophilized to afford 6-((4,6-difluoro-5-(4-(1-hydroxycyclobutyl)phenyl)-1H-benzo[d]imidazol-2-yl)oxy)-5-methylbenzo[c][1,2]oxaborol-1(3H)-ol as white solid (15 mg, Yield: 10% in 2 steps). TLC system: EtOAc/Hexane (100:00), R_{f} value: ˜0.2; LCMS (m/z): 463.44 (M+H)⁺; ¹H NMR (400 MHz, DMSO-d₆) δ: 13.01 (bs, 1H), 9.23 (s, 1H), 7.59-7.57 (m, 3H), 7.40-7.38 (m, 3H), 7.20 (d, J=9.6 Hz, 1H), 5.56 (s, 1H), 5.00 (s, 2H), 2.47-2.40 (m, 2H), 2.33-2.26 (m, 5H), 1.98-1.88 (m, 1H), 1.73-1.62 (m, 1H).

Prep-HPLC condition: Column GEMINI NX C-18 (250*21.2 mm) 5μ Buffer MP-(A): 0.1% FA in WATER MP-(B): ACN:WATER (80:20) Gradient (T/% B) 0/55, 15/75, 25/95 Flow Rate 13 mL/min Solubility WATER, Acetonitrile

### SYNTHETIC EXAMPLE 69

### Synthesis of Compound II-26

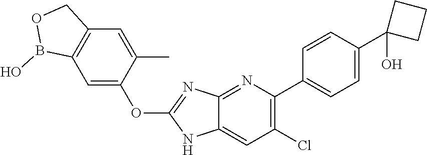 [SMILES: Cc1cc2c(cc1Oc1nc3nc(-c4ccc(C5(O)CCC5)cc4)c(Cl)cc3[nH]1)B(O)OC2]

### Synthesis of 2,4-dihydroxy-5-methylbenzaldehyde

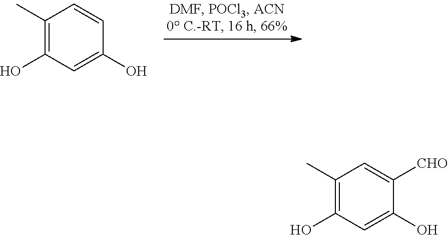

To a stirred solution of 4-methylbenzene-1,3-diol (15 g, 120.9 mmol, 1.0 eq) and DMF (16 mL, 205.6 mmol, 1.7 eq) in ACN (15 Vol) at 0° C., was added POCl₃ (13.5 mL, 145.1 mmol, 1.2 eq) dropwise and stirred at room temperature for 16 h. After completion of reaction by TLC, the reaction mixture was diluted with water (100 mL) and extracted with EtOAc (2×100 mL). The combined organic layer was washed with NaHCO₃ solution (100 mL), dried over Na₂SO₄, filtered and concentrated under reduced pressure to afford crude product. The crude product was purified by silica gel [100-200] column chromatography [elution with 20% EtOAc in petroleum ether] to afford 2,4-dihydroxy-5-methylbenzaldehyde as an off white solid (12 g, Yield: 66%). TLC system: EtOAc/Hexane (20:80), R_{f} value: ˜0.5; LCMS (m/z): 153.11 (M+H)⁺; ¹H NMR (400 MHz, CDCl₃) δ: 11.27 (s, 1H), 9.67 (s, H), 6.35 (s, 1H), 6.06 (s, 1H), 2.20 (s, 3H). One aromatic proton merged with NMR solvent residues.

### Synthesis of 4-(benzyloxy)-2-hydroxy-5-methylbenzaldehyde

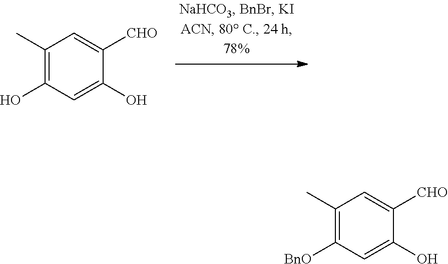 [SMILES: Cc1cc(C=O)c(O)cc1O ; Cc1cc(C=O)c(O)cc1OCc1ccccc1]

To a stirred solution of 2,4-dihydroxy-5-methylbenzaldehyde (12 g, 78.95 mmol, 1.0 eq) in ACN (10 Vol) at room temperature, was added NaHCO₃ (7.96 g, 94.74 mmol, 1.2 eq), KI (1.31 g, 7.89 mmol, 0.1 eq) followed by BnBr (14.85 g, 86.84 mmol, 1.1 eq) and stirred at 80° C. for 24 h. After completion of reaction by TLC, the reaction mixture was diluted with water (150 mL) and extracted with EtOAc (2×100 mL). The combined organic layer was dried over Na₂SO₄, filtered and concentrated under reduced pressure. Obtained crude was purified by silica column [eluted with 20% EtOAc in petroleum ether] to afford 4-(benzyloxy)-2-hydroxy-5-methylbenzaldehyde as a brown gum (15 g, Yield: 78%). TLC system: EtOAc/Hexane (20:80), R_{f} value: ˜0.5; LCMS (m/z): 243.25 (M+H)⁺; ¹H NMR (400 MHz, CDCl₃) δ: 11.41 (s, 1H), 9.69 (s, 1H), 7.42-7.35 (m, 5H), 6.46 (s, 1H), 5.12 (s, 2H), 2.21 (s, 3H).

### Synthesis of 5-(benzyloxy)-2-formyl-4-methylphenyl trifluoromethanesulfonate

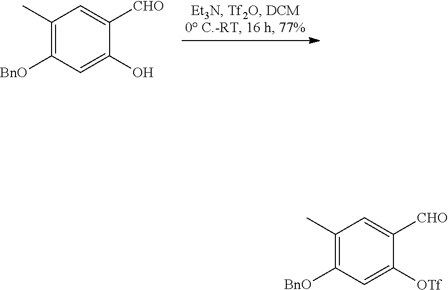 [SMILES: Cc1cc(C=O)c(O)cc1OCc1ccccc1 ; Cc1cc(C=O)c(OS(=O)(=O)C(F)(F)F)cc1OCc1ccccc1]

To a stirred solution of 4-(benzyloxy)-2-hydroxy-5-methylbenzaldehyde (15 g, 61.98 mmol, 1.0 eq) in DCM (20 Vol) at 0° C., was added Et₃N (43 mL, 309.9 mmol, 5.0 eq) followed by Tf₂O (38.4 g, 136.35 mmol, 2.2 eq) and stirred at room temperature for 16 h. After completion of reaction by TLC, the reaction mixture was diluted with water (150 mL) and extracted with DCM (2× 150 mL). The combined organic layer was dried over Na₂SO₄, filtered and concentrated under reduced pressure to afford crude product. The crude product was purified by silica gel [100-200] column chromatography [elution with 10% EtOAc in petroleum ether] to afford 5-(benzyloxy)-2-formyl-4-methylphenyl trifluoromethanesulfonate as a brown gum (18 g, Yield: 77%). TLC system: EtOAc/Hexane (30:70), R_{f} value: ˜0.4; LCMS (m/z): 375.14 (M+H)⁺.

### Synthesis of 4-(benzyloxy)-5-methyl-2-(4,4,5,5-tetramethyl-1,3,2-dioxaborolan-2-yl)benzaldehyde

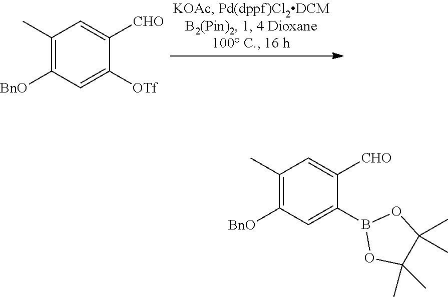 [SMILES: Cc1cc(C=O)c(OS(=O)(=O)C(F)(F)F)cc1OCc1ccccc1 ; Cc1cc(C=O)c(B2OC(C)(C)C(C)(C)O2)cc1OCc1ccccc1]

In a sealed tube, to a degassed solution of 5-(benzyloxy)-2-formyl-4-methylphenyl trifluoromethanesulfonate (18 g, 48.1 mmol, 1.0 eq) and B₂Pin₂ (18.28 g, 72.2 mmol, 1.5 eq) in 1,4-dioxane (20 Vol) at room temperature, was added KOAc (9.44 g, 96.2 mmol, 2 eq) followed by Pd(dppf)Cl₂·DCM (3.92 g, 4.81 mmol, 0.1 eq) and stirred at 100° C. for 16 h. After completion of reaction by TLC, the reaction mixture was diluted with water (150 mL) and extracted with EtOAc (2×150 mL). The combined organic layer was dried over Na₂SO₄, filtered and concentrated under reduced pressure to afford crude product. The crude product was purified by silica gel [100-200] column chromatography [elution with 20% EtOAc in petroleum ether] to afford 4-(benzyloxy)-5-methyl-2-(4,4,5,5-tetramethyl-1,3,2-dioxaborolan-2-yl)benzaldehyde as a yellow gum (9.0 g, semi-pure). TLC system: EtOAc/Hexane (50:50), R_{f} value: ˜0.6; LCMS (m/z): 352.25 (M)⁺.

### Synthesis of 6-(benzyloxy)-5-methylbenzo[c][1,2]oxaborol-1(3H)-ol

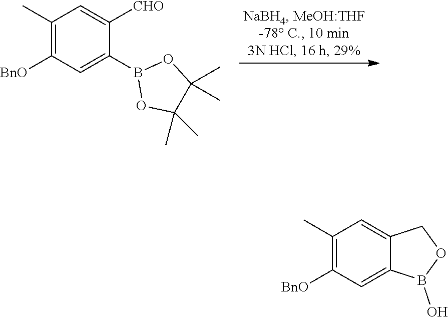 [SMILES: Cc1cc(C=O)c(B2OC(C)(C)C(C)(C)O2)cc1OCc1ccccc1 ; Cc1cc2c(cc1OCc1ccccc1)B(O)OC2]

To a stirred solution of 4-(benzyloxy)-5-methyl-2-(4,4,5,5-tetramethyl-1,3,2-dioxaborolan-2-yl)benzaldehyde (9 g, 25.5 mmol, 1.0 eq) in MeOH: THF (1:1, 10 Vol) at −78° C., was added NaBH₄ (969 mg, 25.5 mmol, 1.0 eq) and stirred for 10 min. After completion of reaction by TLC, the reaction mixture was quenched with 3N aq. HCl solution (10 Vol) and stirred at room temperature for 16 h. Then reaction mixture was diluted with water (80 mL) and extracted with EtOAc (2×80 mL). The combined organic layer was dried over Na₂SO₄, filtered and concentrated under reduced pressure to afford crude product. The crude product was purified by silica gel [100-200] column chromatography [elution with 30% EtOAc in petroleum ether] to afford 6-(benzyloxy)-5-methylbenzo[c][1,2]oxaborol-1(3H)-ol as an off white solid (3.5 g, Yield: 29% in 2 steps). TLC system: EtOAc/Hexane (50:50), R_{f} value: ˜0.4; LCMS (m/z): 255.24 (M+H)⁺.

### Synthesis of 5-methylbenzo[c][1,2]oxaborole-1,6 (3H)-diol

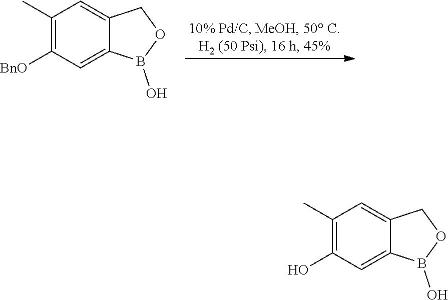 [SMILES: Cc1cc2c(cc1OCc1ccccc1)B(O)OC2 ; Cc1cc2c(cc1O)B(O)OC2]

In a steel bomb, to a stirred solution of 6-(benzyloxy)-5-methylbenzo[c][1,2]oxaborol-1(3H)-ol (3.5 g, 13.74 mmol, 1.0 eq) in MeOH (10 Vol) at room temperature, was added 10% Pd/C (650 mg, 20% wt/wt) and stirred at 50° C. for 16 h under H₂ (50 Psi) atmosphere. After completion of reaction by TLC, the reaction mixture was filtered through a pad of diatomaceous earth (e.g., Celite®), washed with DCM (50 mL) and collected filtrate was concentrated under reduced pressure to afford crude product. The crude product was purified by reverse phase column chromatography [elution with 28% ACN in 0.1% FA in H₂O] to afford 5-methylbenzo[c][1,2]oxaborole-1,6 (3H)-diol as a white solid (1.0 g, Yield: 45%). TLC system: EtOAc/Hexane (50:50), R_{f} value: ˜0.3; LCMS (m/z): 164.11 (M)⁺. ¹H NMR (400 MHz, DMSO-d₆) δ: 9.25 (s, 1H), 8.96 (s, 1H), 7.08 (d, J=6.4 Hz, 2H), 4.82 (s, 2H), 2.16 (s, 3H).

### Synthesis of 6-((6-chloro-5-iodo-1-((2-(trimethylsilyl)ethoxy)methyl)-1H-imidazo[4,5-b]pyridin-2-yl)oxy)-5-methylbenzo[c][1,2]oxaborol-1(3H)-ol

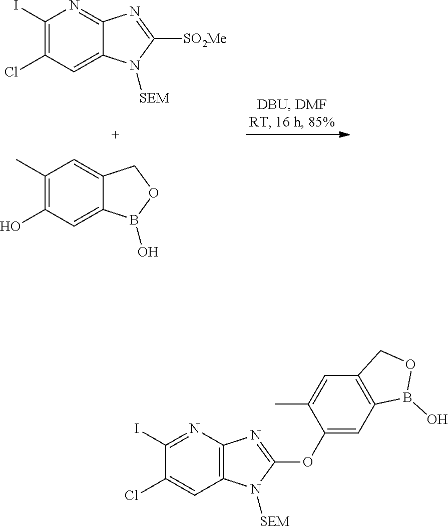 [SMILES: C[Si](C)(C)CCOCn1c(S(C)(=O)=O)nc2nc(I)c(Cl)cc21 ; Cc1cc2c(cc1O)B(O)OC2 ; Cc1cc2c(cc1Oc1nc3nc(I)c(Cl)cc3n1COCC[Si](C)(C)C)B(O)OC2]

To a stirred solution of 6-chloro-5-iodo-2-(methylsulfonyl)-1-((2-(trimethylsilyl)ethoxy)methyl)-1H-imidazo[4,5-b]pyridine (1.0 g, 2.05 mmol, 1.0 eq) in DMF (10 Vol) at room temperature, was added DBU (935 mg, 6.15 mmol, 3.0 eq) followed by 5-methylbenzo[c][1,2]oxaborole-1,6 (3H)-diol (504 mg, 3.07 mmol, 1.5 eq) and stirred at room temperature for 16 h. After completion of reaction by TLC, the reaction mixture was diluted with water (25 mL) and extracted with EtOAc (2×25 mL). The combined organic layer was concentrated under reduced pressure to afford crude product. The crude product was purified by silica gel [100-200] column chromatography [elution with EtOAc to 10% MeOH in DCM] to afford 6-((6-chloro-5-iodo-1-((2-(trimethylsilyl)ethoxy)methyl)-1H-imidazo[4,5-b]pyridin-2-yl)oxy)-5-methylbenzo[c][1,2]oxaborol-1(3H)-ol as a yellow gum (1.0 g, Yield: 85%). TLC system: EtOAc/Hexane (50:50), R_{f} value: ˜0.2; LCMS (m/z): 572.26 (M+H)⁺.

### Synthesis of 6-((6-chloro-5-(4-(1-hydroxycyclobutyl)phenyl)-1-((2-(trimethylsilyl)ethoxy)methyl)-1H-imidazo[4,5-b]pyridin-2-yl)oxy)-5-methylbenzo[c][1,2]oxaborol-1(3H)-ol

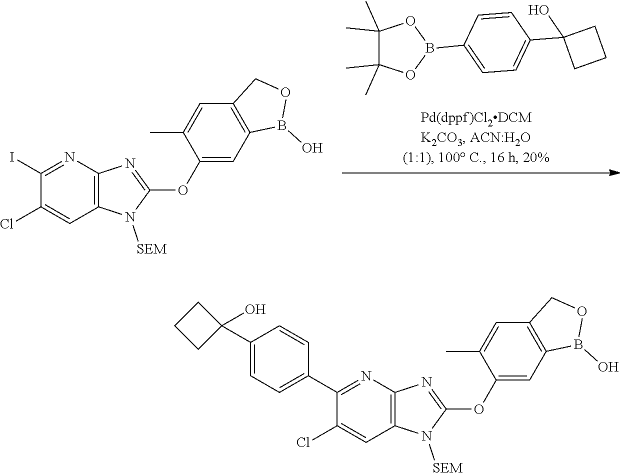 [SMILES: Cc1cc2c(cc1Oc1nc3nc(I)c(Cl)cc3n1COCC[Si](C)(C)C)B(O)OC2 ; CC1(C)OB(c2ccc(C3(O)CCC3)cc2)OC1(C)C ; Cc1cc2c(cc1Oc1nc3nc(-c4ccc(C5(O)CCC5)cc4)c(Cl)cc3n1COCC[Si](C)(C)C)B(O)OC2]

In a sealed tube, to a degassed solution of 6-((6-chloro-5-iodo-1-((2-(trimethylsilyl)ethoxy)methyl)-1H-imidazo[4,5-b]pyridin-2-yl)oxy)-5-methylbenzo[c][1,2]oxaborol-1(3H)-ol (1.0 g, 1.75 mmol, 1.0 eq) and 1-(4-(4,4,5,5-tetramethyl-1,3,2-dioxaborolan-2-yl)phenyl)cyclobutan-1-ol (575 mg, 2.09 mmol, 1.2 eq) in ACN:H₂O (1:1, 10 Vol) at room temperature, was added K₂CO₃ (725 mg, 5.25 mmol, 3.0 eq) followed by Pd(dppf)Cl₂·DCM (143 mg, 0.17 mmol, 0.1 eq) and stirred at 100° C. for 16 h. After completion of reaction by TLC, the reaction mixture was diluted with water (25 mL) and extracted with EtOAc (2×25 mL). The combined organic layer was concentrated under reduced pressure to afford crude product. The crude product was purified by reverse phase column chromatography [elution with 70% ACN in 0.1% FA in H₂O] to afford 6-((6-chloro-5-(4-(1-hydroxycyclobutyl)phenyl)-1-((2-(trimethylsilyl)ethoxy)methyl)-1H-imidazo[4,5-b]pyridin-2-yl)oxy)-5-methylbenzo[c][1,2]oxaborol-1(3H)-ol as brown solid (200 mg, Yield: 20%). TLC system: EtOAc/Hexane (100:00), R_{f} value: ˜0.3; LCMS (m/z): 592.38 (M+H)⁺.

### Synthesis of 6-((6-chloro-5-(4-(1-hydroxycyclobutyl)phenyl)-1H-imidazo[4,5-b]pyridin-2-yl)oxy)-5-methylbenzo[c][1,2]oxaborol-1(3H)-ol

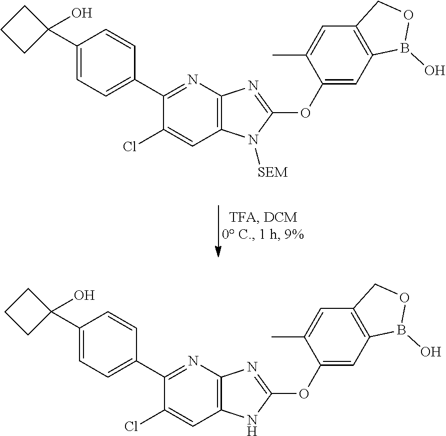 [SMILES: Cc1cc2c(cc1Oc1nc3nc(-c4ccc(C5(O)CCC5)cc4)c(Cl)cc3n1COCC[Si](C)(C)C)B(O)OC2 ; Cc1cc2c(cc1Oc1nc3nc(-c4ccc(C5(O)CCC5)cc4)c(Cl)cc3[nH]1)B(O)OC2]

To a stirred solution of 6-((6-chloro-5-(4-(1-hydroxycyclobutyl)phenyl)-1-((2-(trimethylsilyl)ethoxy)methyl)-1H-imidazo[4,5-b]pyridin-2-yl)oxy)-5-methylbenzo[c][1,2]oxaborol-1(3H)-ol (230 mg, 0.38 mmol, 1.0 eq) in DCM (10 Vol) at 0° C., was added TFA (1.15 mL, 5 Vol) and stirred at 0° C. for 1 h. After completion of reaction by TLC, the reaction mixture was concentrated under reduced pressure and purified by Prep-HPLC, collected fractions were lyophilized to afford desired product. This was re-purified by reverse phase column chromatography [elution with 56% ACN in 0.1% FA in H₂O] to afford 6-((6-chloro-5-(4-(1-hydroxycyclobutyl)phenyl)-1H-imidazo[4,5-b]pyridin-2-yl)oxy)-5-methylbenzo[c][1,2]oxaborol-1(3H)-ol as white solid (16 mg, Yield: 9%). TLC system: MeOH/DCM (20:80), R_{f} value: ˜0.1; LCMS (m/z): 460.22 (M−H)⁻; ¹H NMR (400 MHz, DMSO-d₆) δ: 13.26 (bs, 1H), 9.24 (s, 1H), 7.92 (s, 1H), 7.63-7.56 (m, 5H), 7.41 (s, 1H), 5.58 (s, 1H), 5.00 (s, 2H), 2.49-2.41 (m, 2H), 2.34-2.29 (m, 2H), 2.25 (s, 3H), 1.99-1.93 (m, 1H), 1.75-1.66 (m, 1H).

Prep-HPLC condition: Column YMC C-18 (250·21.2 mm) 5μ Buffer MP-(A): 10 Mm ammonium acetate MP-(B): ACN: water (80:20) Gradient (T/% B) 0/40, 11/65, 15/65, 25/95 Flow Rate 13 mL/min Solubility ACN&WATER

### SYNTHETIC EXAMPLE 70

### Synthesis of Compound IV-5

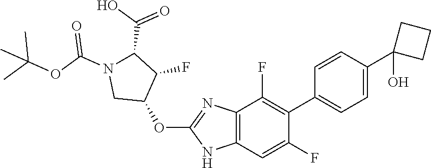 [SMILES: CC(C)(C)OC(=O)N1C[C@@H](Oc2nc3c(F)c(-c4ccc(C5(O)CCC5)cc4)c(F)cc3[nH]2)[C@@H](F)[C@H]1C(=O)O]

### Synthesis of 1-(3′,4′-diamino-2′,6′-difluoro-[1,1′-biphenyl]-4-yl)cyclobutan-1-ol

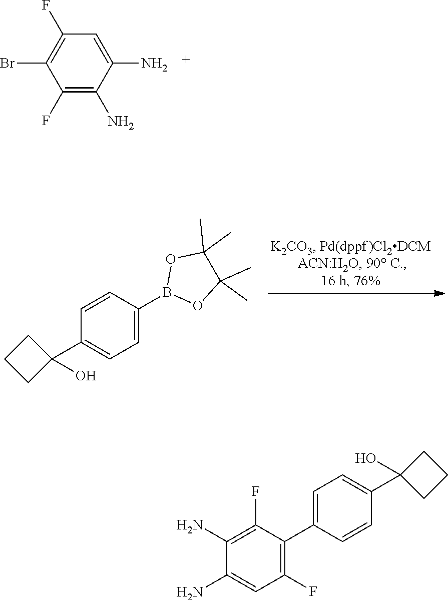 [SMILES: Nc1cc(F)c(Br)c(F)c1N ; CC1(C)OB(c2ccc(C3(O)CCC3)cc2)OC1(C)C ; Nc1cc(F)c(-c2ccc(C3(O)CCC3)cc2)c(F)c1N]

In a sealed tube, to a degassed solution of 4-bromo-3,5-difluorobenzene-1,2-diamine (3 g, 13.5 mmol, 1.0 eq) and 1-(4-(4,4,5,5-tetramethyl-1,3,2-dioxaborolan-2-yl)phenyl)cyclobutan-1-ol (4.44 g, 16.2 mmol, 1.2 eq) in ACN:H₂O (8:2, 10 Vol) at room temperature, was added K₂CO₃ (5.59 g, 40.54 mmol, 3.0 eq) followed by Pd(dppf)Cl₂·DCM (0.55 g, 0.67 mmol, 0.05 eq) and stirred at 90° C. for 16 h. After completion of reaction by TLC, the reaction mixture was diluted with water (60 mL) and extracted with EtOAc (2×30 mL). The combined organic layer was dried over Na₂SO₄, filtered and concentrated under reduced pressure. The crude product was purified by silica gel [100-200] column chromatography [elution with 50% EtOAc in hexane] to afford 1-(3′,4′-diamino-2′,6′-difluoro-[1,1′-biphenyl]-4-yl)cyclobutan-1-ol as a yellow solid (3 g, Yield: 76%). TLC system: EtOAc/Hexane (50:50), R_{f} value: ˜0.3; LCMS (m/z): 291.3 (M+H)⁺; ¹H NMR (400 MHz, CDCl₃) δ: 7.56 (d, J=8.4 Hz, 2H), 7.45 (d, J=8.4 Hz, 2H), 6.36 (dd, J=11.2 Hz, 2 Hz, 1H), 2.65-2.60 (m, 2H), 2.43-2.36 (m, 2H), 2.10-2.00 (m, 1H), 1.76-1.71 (m, 1H), aliphatic impurities observed.

### Synthesis of 4,6-difluoro-5-(4-(1-hydroxycyclobutyl)phenyl)-1,3-dihydro-2H-benzo[d]imidazole-2-thione

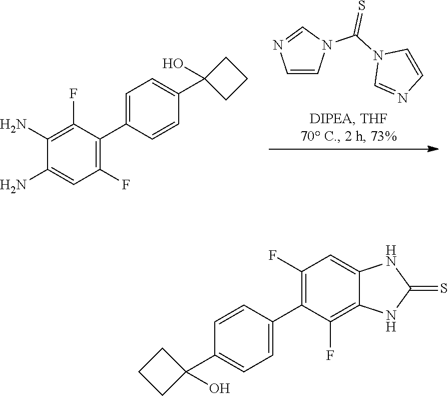

To a stirred solution of 1-(3′,4′-diamino-2′,6′-difluoro-[1,1′-biphenyl]-4-yl)cyclobutan-1-ol (3 g, 10.34 mmol, 1.0 eq) in THF (30 mL, 10 Vol) at room temperature, was added DIPEA (5.7 mL, 31.03 mmol, 3.0 eq) followed by Thio-CDI (2.76 g, 15.51 mmol, 1.5 eq) and stirred at 70° C. for 2 h. After completion of reaction by TLC, the reaction mixture was diluted with water (60 mL) and extracted with EtOAc (2×30 mL). The combined organic layer was dried over Na₂SO₄, filtered and concentrated under reduced pressure to afford crude product. The crude product was purified by silica gel [100-200] column chromatography [elution with 30% EtOAc in hexane] to afford 4,6-difluoro-5-(4-(1-hydroxycyclobutyl)phenyl)-1,3-dihydro-2H-benzo[d]imidazole-2-thione as a yellow solid (2.5 g, Yield: 73%). TLC system: EtOAc/Hexane (50:50), R_{f} value: ˜0.7; LCMS (m/z): 333.3 (M+H)⁺.

### Synthesis of 1-(4-(5,7-difluoro-2-(methylthio)-1H-benzo[d]imidazol-6-yl)phenyl)cyclobutan-1-ol

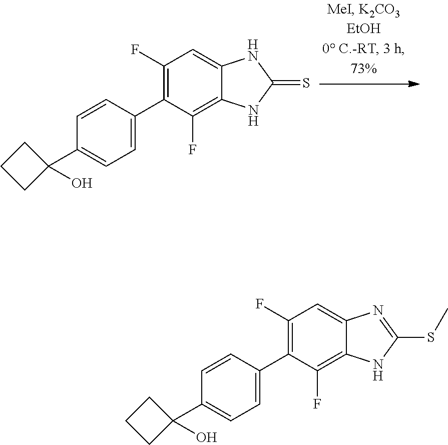 [SMILES: OC1(c2ccc(-c3c(F)cc4[nH]c(=S)[nH]c4c3F)cc2)CCC1 ; CSc1nc2cc(F)c(-c3ccc(C4(O)CCC4)cc3)c(F)c2[nH]1]

To a stirred solution of 4,6-difluoro-5-(4-(1-hydroxycyclobutyl)phenyl)-1,3-dihydro-2H-benzo[d]imidazole-2-thione (2.5 g, 7.53 mmol, 1.0 eq) in EtOH (50 mL, 20 Vol) at 0° C., was added K₂CO₃ (1.55 g, 11.30 mmol, 1.5 eq) followed by MeI (0.4 mL, 7.53 mmol, 1.0 eq) and stirred at room temperature for 3 h. After completion of reaction by TLC, the reaction mixture was concentrated, diluted with water (50 mL) and extracted with EtOAc (2×25 mL). The combined organic layer was dried over Na₂SO₄, filtered and concentrated under reduced pressure. The crude product was purified by silica gel [100-200 mesh] column chromatography [elution with 35% EtOAc in hexane] to afford 1-(4-(5,7-difluoro-2-(methylthio)-1H-benzo[d]imidazol-6-yl)phenyl)cyclobutan-1-ol as pale yellow solid (1.9 g, Yield: 73%). TLC system: EtOAc/Hexane (50:50), R_{f} value: ˜0.6; LCMS (m/z): 347.38 (M+H)⁺; ¹H NMR (400 MHz, CDCl₃) δ: 7.61 (d, J=8.4 Hz, 2H), 7.50 (d, J=8.0 Hz, 2H), 7.12 (d, J=8.8 Hz, 1H), 2.81 (s, 3H), 2.65-2.60 (m, 2H), 2.45-2.40 (m, 2H), 2.07-2.05 (s, 1H), 1.80-1.72 (m, 1H), EtOAc solvent residues observed.

### Synthesis of 1-(4-(5,7-difluoro-2-(methylsulfonyl)-1H-benzo[d]imidazol-6-yl)phenyl)cyclobutan-1-ol

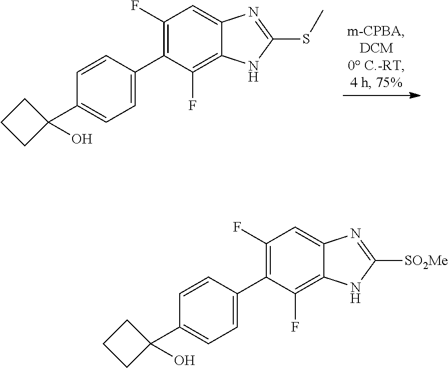 [SMILES: CSc1nc2cc(F)c(-c3ccc(C4(O)CCC4)cc3)c(F)c2[nH]1 ; CS(=O)(=O)c1nc2cc(F)c(-c3ccc(C4(O)CCC4)cc3)c(F)c2[nH]1]

To a stirred solution of 1-(4-(5,7-difluoro-2-(methylthio)-1H-benzo[d]imidazol-6-yl)phenyl)cyclobutan-1-ol (1.9 g, 5.49 mmol, 1.0 eq) in DCM (19 mL, 10 Vol) at 0° C., was added m-CPBA (2.36 g, 13.7 mmol, 2.5 eq) and stirred at room temperature for 4 h. After completion of reaction by TLC, the reaction mixture was diluted with water (30 mL) and extracted with EtOAc (2×30 mL). The combined organic layer was washed with NaHCO₃ solution, dried over Na₂SO₄, filtered and concentrated under reduced pressure. The crude product was purified by silica gel [100-200 mesh] column chromatography [elution with 30% EtOAc in hexane] to afford 1-(4-(5,7-difluoro-2-(methylsulfonyl)-1H-benzo[d]imidazol-6-yl)phenyl)cyclobutan-1-ol as off white solid (1.5 g, Yield: 75%). TLC system: EtOAc/Hexane (50:50), R_{f} value: ˜0.5; LCMS (m/z): 379.38 (M+H)⁺;

### Synthesis of 1-(4-(5,7-difluoro-2-(methylsulfonyl)-1-((2-(trimethylsilyl)ethoxy)methyl)-1H-benzo[d]imidazol-6-yl)phenyl)cyclobutan-1-ol

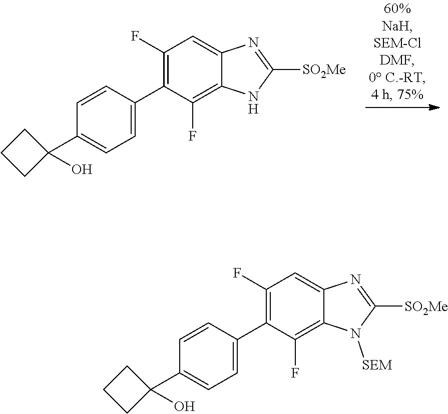 [SMILES: CS(=O)(=O)c1nc2cc(F)c(-c3ccc(C4(O)CCC4)cc3)c(F)c2[nH]1 ; C[Si](C)(C)CCOCn1c(S(C)(=O)=O)nc2cc(F)c(-c3ccc(C4(O)CCC4)cc3)c(F)c21]

To a stirred solution of 1-(4-(5,7-difluoro-2-(methylsulfonyl)-1H-benzo[d]imidazol-6-yl)phenyl)cyclobutan-1-ol (1.5 g, 3.96 mmol, 1.0 eq) in DMF (10 Vol) at 0° C., was added 60% NaH (190 mg, 7.93 mmol, 2 eq) and stirred for 30 min. Then SEM-Cl (1 mL, 5.95 mmol, 1.5 eq) was added to reaction mixture and stirred at room temperature for 4 h. After completion of reaction by TLC, the reaction mixture was quenched with water (30 mL) and extracted with EtOAc (2×20 mL). The combined organic layer was dried over Na₂SO₄, filtered and concentrated under reduced pressure. The crude product was purified by silica gel [100-200] column chromatography [elution with 20% EtOAc in hexane] to afford 1-(4-(5,7-difluoro-2-(methylsulfonyl)-1-((2-(trimethylsilyl)ethoxy)methyl)-1H-benzo[d]imidazol-6-yl)phenyl)cyclobutan-1-ol as off white solid (1.5 g, Yield: 75%). TLC system: EtOAc/Hexane (40:60), R_{f} value: ˜0.6; LCMS (m/z): 509.5 (M+H)⁺, mixture of regio-isomers.

### Synthesis of (2R,3S,4R)-1-(tert-butoxycarbonyl)-4-((4,6-difluoro-5-(4-(1-hydroxycyclobutyl)phenyl)-1-((2-(trimethylsilyl)ethoxy)methyl)-1H-benzo[d]imidazol-2-yl)oxy)-3-fluoropyrrolidine-2-carboxylic acid

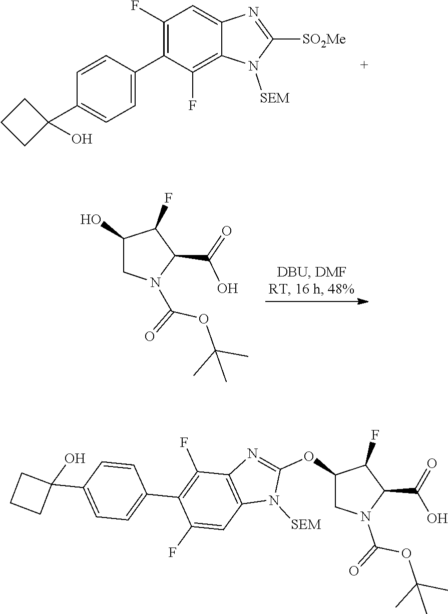 [SMILES: C[Si](C)(C)CCOCn1c(S(C)(=O)=O)nc2cc(F)c(-c3ccc(C4(O)CCC4)cc3)c(F)c21 ; CC(C)(C)OC(=O)N1C[C@@H](O)[C@@H](F)[C@H]1C(=O)O ; CC(C)(C)OC(=O)N1C[C@@H](Oc2nc3c(F)c(-c4ccc(C5(O)CCC5)cc4)c(F)cc3n2COCC[Si](C)(C)C)[C@@H](F)[C@H]1C(=O)O]

To a stirred solution of 1-(4-(5,7-difluoro-2-(methylsulfonyl)-1-((2-(trimethylsilyl)ethoxy)methyl)-1H-benzo[d]imidazol-6-yl)phenyl)cyclobutan-1-ol (200 mg, 0.39 mmol, 1.0 eq) in DMF (2 mL, 10 vol) at room temperature, was added DBU (0.1 mL, 1.18 mmol, 3.0 eq) followed by (2R,3S,4R)-1-(tert-butoxycarbonyl)-3-fluoro-4-hydroxypyrrolidine-2-carboxylic acid (117 mg, 0.55 mmol, 1.2 eq) and stirred at room temperature for 16 h. After completion of reaction by TLC, the reaction mixture was concentrated under reduced pressure and purified by silica gel [100-200] column chromatography [elution with 5% MeOH in DCM] to afford (2R,3S,4R)-1-(tert-butoxycarbonyl)-4-((4,6-difluoro-5-(4-(1-hydroxycyclobutyl)phenyl)-1-((2-(trimethylsilyl)ethoxy)methyl)-1H-benzo[d]imidazol-2-yl)oxy)-3-fluoropyrrolidine-2-carboxylic acid off white solid (130 mg, Yield: 48%). TLC system: MeOH/DCM (10:90), R_{f} value: ˜0.4; LCMS (m/z): 678.5 (M+H)⁺; regio-isomers from SEM protection.

### Synthesis of (2R,3S,4R)-1-(tert-butoxycarbonyl)-4-((4,6-difluoro-5-(4-(1-hydroxycyclobutyl)phenyl)-1H-benzo[d]imidazol-2-yl)oxy)-3-fluoropyrrolidine-2-carboxylic acid

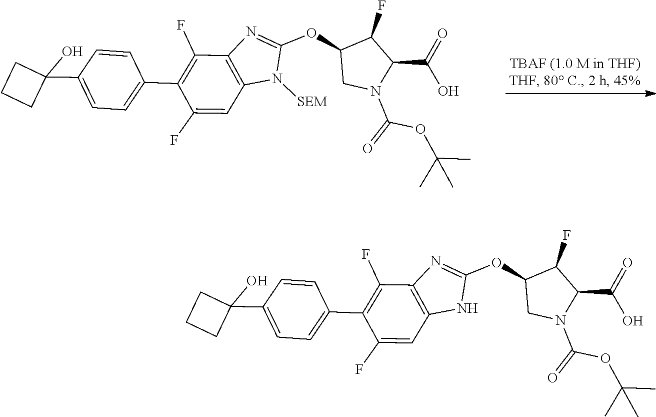 [SMILES: CC(C)(C)OC(=O)N1C[C@@H](Oc2nc3c(F)c(-c4ccc(C5(O)CCC5)cc4)c(F)cc3n2COCC[Si](C)(C)C)[C@@H](F)[C@H]1C(=O)O ; CC(C)(C)OC(=O)N1C[C@@H](Oc2nc3c(F)c(-c4ccc(C5(O)CCC5)cc4)c(F)cc3[nH]2)[C@@H](F)[C@H]1C(=O)O]

To a stirred solution of (2R,3S,4R)-1-(tert-butoxycarbonyl)-4-((4,6-difluoro-5-(4-(1-hydroxycyclobutyl)phenyl)-1-((2-(trimethylsilyl)ethoxy)methyl)-1H-benzo[d]imidazol-2-yl)oxy)-3-fluoropyrrolidine-2-carboxylic acid (130 mg, 0.192 mmol, 1.0 eq) in THF (1.3 mL, 10 Vol) at 0° C., was added TBAF (1.0 M in THF) (1.3 mL, 10 Vol) and stirred at 80° C. for 2 h. After completion of reaction by TLC, the reaction mixture was quenched with NH₄Cl solution (5 mL) and extracted with 10% MeOH in DCM (2× 10 mL). The combined organic layer was dried over Na₂SO₄, filtered and concentrated under reduced pressure. The crude product was purified by Prep-HPLC, collected fractions were lyophilized to afford (2R,3S,4R)-1-(tert-butoxycarbonyl)-4-((4,6-difluoro-5-(4-(1-hydroxycyclobutyl)phenyl)-1H-benzo[d]imidazol-2-yl)oxy)-3-fluoropyrrolidine-2-carboxylic acid off white solid (45 mg, Yield: 42%). TLC system: MeOH/DCM (10:90), R_{f} value: ˜0.1; LCMS (m/z): 548.3 (M+H)⁺; ¹H NMR (400 MHz, DMSO-d₆) δ: 13.27 (bs, 2H) (D₂O Exchangeable protons), 7.59 (d, J=8.4 Hz, 2H), 7.41 (d, J=8 Hz, 2H), 7.15 (br, 1H), 5.73-5.48 (m, 3H) (1 proton D₂O Exchangeable), 4.68-4.59 (m, 1H), 4.24-4.17 (m, 1H), 3.46-3.42 (m, 1H), 2.45-2.43 (m, 2H), 2.33-2.30 (m, 2H), 1.98-1.90 (m, 1H), 1.72-1.65 (m, 1H), 1.43-1.37 (s→d, 9H).

Prep-HPLC condition: Column GEMINI NX C-18 (250*21.2 mm) 5μ Buffer MP-(A): 0.1% FA in WATER MP-(B): ACN Gradient (T/% B) 0/40, 15/75, 25/95 Flow Rate 13 mL/min Solubility ACN, WATER.

### SYNTHETIC EXAMPLE 71

### Synthesis of Compound II-27

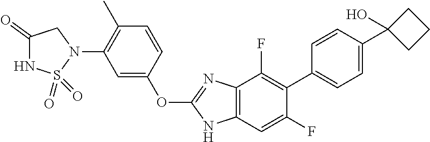 [SMILES: Cc1ccc(Oc2nc3c(F)c(-c4ccc(C5(O)CCC5)cc4)c(F)cc3[nH]2)cc1N1CC(=O)NS1(=O)=O]

### Synthesis of 5-((tert-butyldimethylsilyl)oxy)-2-methylaniline

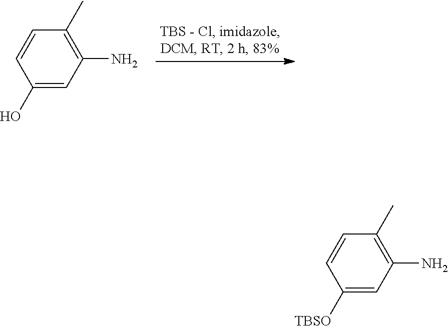

To a stirred solution of 3-amino-4-methylphenol (1.5 g, 12.2 mmol, 1.0 eq) in DCM (15 mL, 10 Vol) at room temperature, was added imidazole (995 mg, 14.6 mmol, 1.2 eq), TBS-Cl (2.2 g, 14.6 mmol, 1.2 eq) and stirred at room temperature for 2 h. After completion of reaction by TLC, the reaction mixture was diluted with water (50 mL) and extracted with DCM (2×35 mL). The combined organic layer was dried over Na₂SO₄, filtered and concentrated under reduced pressure to afford crude product. The crude product was purified by silica gel [60-120] column chromatography [elution with 10% EtOAc in petroleum ether] to afford 5-((tert-butyldimethylsilyl)oxy)-2-methylaniline as a yellow gummy (2.4 g, Yield: 83%). TLC system: EtOAc/Hexane (10:90), R_{f} value: ˜0.5; LCMS (m/z): 238.25 (M+H)⁺; ¹H NMR (400 MHz, CDCl₃) δ: 6.87-6.85 (m, 1H), 6.22-6.19 (m, 2H) 2.42 (br s, 2H), 2.09 (s, 3H), 0.96 (s, 9H), 0.17 (s, 6H).

### Synthesis of ethyl (5-((tert-butyldimethylsilyl)oxy)-2-methylphenyl)glycinate

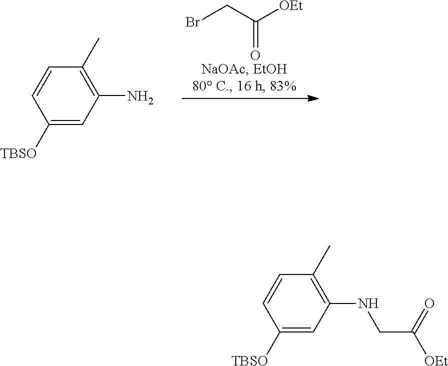 [SMILES: Cc1ccc(O[Si](C)(C)C(C)(C)C)cc1N ; CCOC(=O)CBr ; CCOC(=O)CNc1cc(O[Si](C)(C)C(C)(C)C)ccc1C]

To a stirred solution of 5-((tert-butyldimethylsilyl)oxy)-2-methylaniline (2.4 g, 10.08 mmol, 1.0 eq) in EtOH (24 mL, 10 Vol) at room temperature, was added NaOAc (1.9 g, 23.19 mmol, 2.3 eq) followed by ethyl 2-bromoacetate (2.02 g, 12.10 mmol, 1.2 eq) and stirred at 80° C. for 16 h. After completion of reaction by TLC, the reaction mixture was diluted with water (60 mL) and extracted with EtOAc (2×45 mL). The combined organic layer was dried over Na₂SO₄, filtered and concentrated under reduced pressure to afford crude product. The crude product was purified by silica gel [100-200] column chromatography [elution with 10% EtOAc in petroleum ether] to afford ethyl (5-((tert-butyldimethylsilyl)oxy)-2-methylphenyl)glycinate as a brown gummy (2.7 g, Yield: 83%). TLC system: EtOAc/Hexane (10:90), R_{f} value: ˜0.6; LCMS (m/z): 324.39 (M+H)⁺; ¹H NMR (400 MHz, CDCl₃) δ: 6.89 (d, J=8.0 Hz, 1H), 6.18 (dd, J=2.4 & 8.0 Hz, 1H), 6.00 (d, J=2.0 Hz, 1H), 4.26 (q, J=7.2 Hz, 2H), 3.89 (s, 3H), 2.13 (s, 3H), 1.31 (t, J=7.2 Hz, 3H), 0.97 (s, 9H), 0.18 (s, 6H).

### Synthesis of ethyl N-(N-(tert-butoxycarbonyl)sulfamoyl)-N-(5-((tert-butyldimethylsilyl)oxy)-2-methylphenyl)glycinate

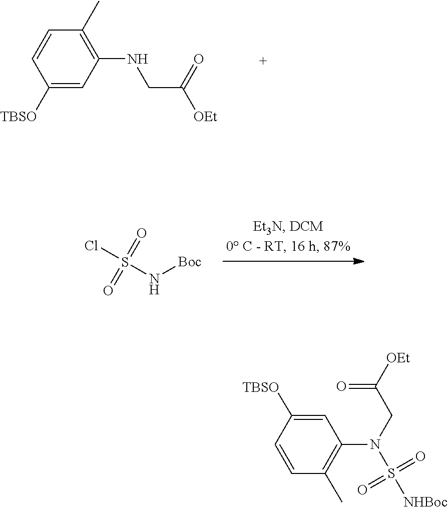 [SMILES: CCOC(=O)CNc1cc(O[Si](C)(C)C(C)(C)C)ccc1C ; CC(C)(C)OC(=O)NS(=O)(=O)Cl ; CCOC(=O)CN(c1cc(O[Si](C)(C)C(C)(C)C)ccc1C)S(=O)(=O)NC(=O)OC(C)(C)C]

To a stirred solution of ethyl (5-((tert-butyldimethylsilyl)oxy)-2-methylphenyl)glycinate (2.7 g, 8.35 mmol, 1.0 eq) in DCM (54 mL, 20 Vol) at 0° C., was added Et₃N (3.5 mL, 25.07 mmol, 3.0 eq) followed by tert-butyl (chlorosulfonyl)carbamate (5.4 g, 25.07 mmol, 3.0 eq) and stirred for 16 h at room temperature. After completion of reaction by TLC, reaction mixture was diluted with water (30 mL) and extracted with DCM (2×40 mL). The combined organic layer was dried over Na₂SO₄, filtered and concentrated under reduced pressure. The crude product was purified by silica gel [100-200] column chromatography [elution with 20% EtOAc in petroleum ether] afforded ethyl N-(N-(tert-butoxycarbonyl)sulfamoyl)-N-(5-((tert-butyldimethylsilyl)oxy)-2-methylphenyl)glycinate as a brown gummy (3.6 g, Yield: 87%). TLC system: EtOAc/Hexane (10:90), R_{f} value: ˜0.2; LCMS (m/z): 501.35 (M−H)⁺.

### Synthesis of ethyl N-(5-((tert-butyldimethylsilyl)oxy)-2-methylphenyl)-N-sulfamoylglycinate

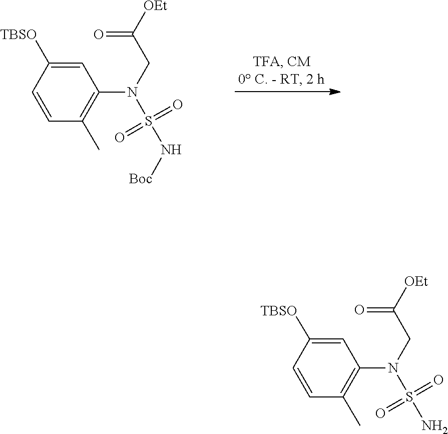 [SMILES: CCOC(=O)CN(c1cc(O[Si](C)(C)C(C)(C)C)ccc1C)S(=O)(=O)NC(=O)OC(C)(C)C ; CCOC(=O)CN(c1cc(O[Si](C)(C)C(C)(C)C)ccc1C)S(N)(=O)=O]

To a stirred solution of ethyl N-(N-(tert-butoxycarbonyl)sulfamoyl)-N-(5-((tert-butyldimethylsilyl)oxy)-2-methylphenyl)glycinate (3.6 g, 7.17 mmol, 1.0 eq) in DCM (72 mL, 20 Vol) at 0° C., was added TFA (18 mL, 5 Vol) and stirred at room temperature for 2 h. After completion of reaction by TLC, reaction mixture was concentrated, diluted with sat. NaHCO₃ solution (30 mL) and extracted with EtOAc (2×100 mL). The combined organic layer was dried over Na₂SO₄, filtered and concentrated under reduced to afford ethyl N-(5-((tert-butyldimethylsilyl)oxy)-2-methylphenyl)-N-sulfamoylglycinate as pale brown gummy (3.0 g, crude). TLC system: EtOAc/Hexane (30:70), R_{f} value: ^{˜}0.4; LCMS (m/z): 403.37 [M+H]⁺; ¹H NMR (400 MHz, CDCl₃) δ: 7.14 (d, J=8.4 Hz, 1H), 7.10 (d, J=2.4 Hz, 1H), 6.79 (dd, J=2.4 & 8.4 Hz, 1H), 5.19 (s, 2H), 4.23 (q, J=7.2 Hz, 2H), 2.30 (s, 3H), 1.32-1.25 (m, 5H), 0.96 (s, 9H), 0.18 (s, 6H).

### Synthesis of ethyl N-(5-hydroxy-2-methylphenyl)-N-sulfamoylglycinate

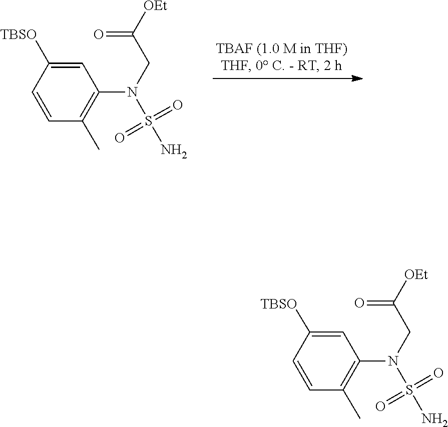 [SMILES: CCOC(=O)CN(c1cc(O[Si](C)(C)C(C)(C)C)ccc1C)S(N)(=O)=O ; CCOC(=O)CN(c1cc(O[Si](C)(C)C(C)(C)C)ccc1C)S(N)(=O)=O]

To a stirred solution of ethyl N-(5-((tert-butyldimethylsilyl)oxy)-2-methylphenyl)-N-sulfamoylglycinate (3.0 g, 7.46 mmol, 1.0 eq) in THF (30 mL, 10 Vol) at 0° C., was added TBAF (1.0 M in THF) (6 mL, 2 Vol) and stirred at room temperature for 2 h. After completion of reaction by TLC, the reaction mixture was quenched with NH₄Cl solution (30 mL) and extracted with EtOAc (2×100 mL). The combined organic layer was dried over Na₂SO₄, filtered and concentrated under reduced pressure to afford ethyl N-(5-hydroxy-2-methylphenyl)-N-sulfamoylglycinate as a pale brown gummy (2.0 g, crude). TLC system: EtOAc/Hexane (30:70), R_{f} value: ^{˜}0.2; LCMS (m/z): 289.24 [M+H]⁺.

### Synthesis of 5-(5-hydroxy-2-methylphenyl)-1,2,5-thiadiazolidin-3-one 1,1-dioxide

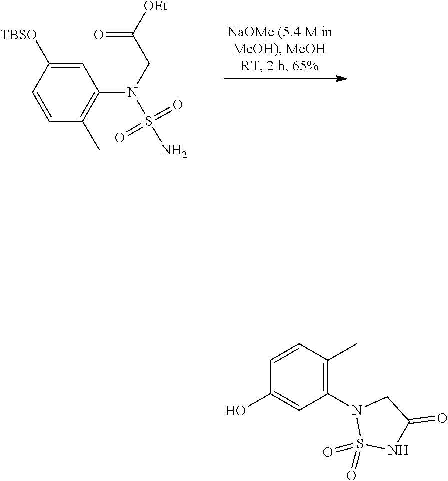 [SMILES: CCOC(=O)CN(c1cc(O[Si](C)(C)C(C)(C)C)ccc1C)S(N)(=O)=O ; Cc1ccc(O)cc1N1CC(=O)NS1(=O)=O]

To a stirred solution of ethyl N-(5-hydroxy-2-methylphenyl)-N-sulfamoylglycinate (2.0 g, 6.94 mmol, 1.0 eq) in MeOH (20 mL, 10 Vol) at room temperature, was added NaOMe (5.4 M in MeOH) (1 Vol) and stirred for 2 h. After completion of reaction by TLC, the reaction mixture was concentrated under reduced pressure and purified by reverse phase column chromatography [elution with 5% ACN in 0.1% FA in H₂O] to afford 5-(5-hydroxy-2-methylphenyl)-1,2,5-thiadiazolidin-3-one 1,1-dioxide as off-white solid (1.1 g, Yield: 65%). TLC system: MeOH/DCM (10:90), R_{f} value: ˜0.1 (Not lifted); ¹H NMR (400 MHz, DMSO-d₆) δ: 9.39 (s, 1H), 8.51 (s, 1H), 6.98 (d, J=8.4 Hz, 1H), 6.81 (d, J=2.4 Hz, 1H), 6.58 (d, J=2.4 & 8.4 Hz, 1H), 3.84 (s, 2H), 2.18 (s, 3H).

### Synthesis of 5-(5-((4,6-difluoro-5-(4-(1-hydroxycyclobutyl)phenyl)-1-((2-(trimethylsilyl)ethoxy)methyl)-1H-benzo[d]imidazol-2-yl)oxy)-2-methylphenyl)-1,2,5-thiadiazolidin-3-one 1,1-dioxide

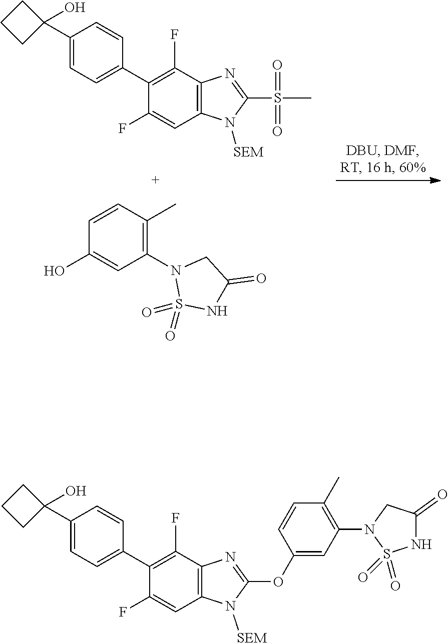 [SMILES: C[Si](C)(C)CCOCn1c(S(C)(=O)=O)nc2c(F)c(-c3ccc(C4(O)CCC4)cc3)c(F)cc21 ; Cc1ccc(O)cc1N1CC(=O)NS1(=O)=O ; Cc1ccc(Oc2nc3c(F)c(-c4ccc(C5(O)CCC5)cc4)c(F)cc3n2COCC[Si](C)(C)C)cc1N1CC(=O)NS1(=O)=O]

To a stirred solution of 1-(4-(4,6-difluoro-2-(methylsulfonyl)-1-((2-(trimethylsilyl)ethoxy)methyl)-1H-benzo[d]imidazol-5-yl)phenyl)cyclobutan-1-ol (250 mg, 0.50 mmol, 1.0 eq) in DMF (2.5 mL, 10 vol) at room temperature, was added DBU (225 mg, 1.47 mmol, 3.0 eq) followed by 5-(5-hydroxy-2-methylphenyl)-1,2,5-thiadiazolidin-3-one 1,1-dioxide (180 mg, 0.74 mmol, 1.5 eq) and stirred at room temperature for 16 h. After completion of reaction by TLC, the reaction mixture was quenched with NH₄Cl solution (20 mL) and extracted with EtOAc (2×20 mL). The combined organic layer was dried over Na₂SO₄, filtered and concentrated under reduced pressure to afford crude product. The crude product was purified by silica gel [100-200] column chromatography [elution with 15% MeOH in DCM] to afford 5-(5-((4,6-difluoro-5-(4-(1-hydroxycyclobutyl)phenyl)-1-((2-(trimethylsilyl)ethoxy)methyl)-1H-benzo[d]imidazol-2-yl)oxy)-2-methylphenyl)-1,2,5-thiadiazolidin-3-one 1,1-dioxide as off white solid (200 mg, Yield: 60%). TLC system: MeOH/DCM (10:90), R_{f} value: ˜0.1; LCMS (m/z): 671.3 (M+H)⁺; mixture of regio-isomers.

### Synthesis of 5-(5-((4,6-difluoro-5-(4-(1-hydroxycyclobutyl)phenyl)-1H-benzo[d]imidazol-2-yl)oxy)-2-methylphenyl)-1,2,5-thiadiazolidin-3-one 1,1-dioxide

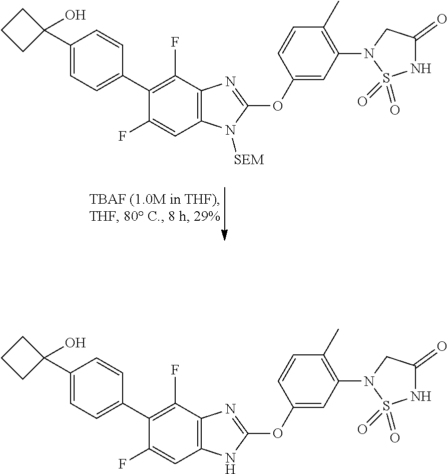 [SMILES: Cc1ccc(Oc2nc3c(F)c(-c4ccc(C5(O)CCC5)cc4)c(F)cc3n2COCC[Si](C)(C)C)cc1N1CC(=O)NS1(=O)=O ; Cc1ccc(Oc2nc3c(F)c(-c4ccc(C5(O)CCC5)cc4)c(F)cc3[nH]2)cc1N1CC(=O)NS1(=O)=O]

To a stirred solution of 5-(5-((4,6-difluoro-5-(4-(1-hydroxycyclobutyl)phenyl)-1-((2-(trimethylsilyl)ethoxy)methyl)-1H-benzo[d]imidazol-2-yl)oxy)-2-methylphenyl)-1,2,5-thiadiazolidin-3-one 1,1-dioxide (200 mg, 0.30 mmol, 1.0 eq) in THF (2 mL, 10 Vol) at 0° C., was added TBAF (1.0 M in THF) (2 mL, 10 Vol) and stirred at 80° C. for 8 h. After completion of reaction by TLC, the reaction mixture was quenched with NH₄Cl solution (20 mL) and extracted with EtOAc (2×20 mL). The combined organic layer was dried over Na₂SO₄, filtered and concentrated under reduced pressure to afford crude product. The crude product was purified triturated with n-pentane: Et₂₀ (1:1) (20 mL), purified by Prep-HPLC, collected fractions were frozen and lyophilized to afford desired product with salts. Re-purified by reverse phase column chromatography [elution with 40% ACN in 0.1% FA in H₂O] afforded 5-(5-((4,6-difluoro-5-(4-(1-hydroxycyclobutyl)phenyl)-1H-benzo[d]imidazol-2-yl)oxy)-2-methylphenyl)-1,2,5-thiadiazolidin-3-one 1,1-dioxide as a white solid (48 mg, Yield: 29%). TLC system: MeOH/DCM (20:80), R_{f} value: ˜0.5; LCMS (m/z): 541.39 (M+H)⁺;

¹H NMR (400 MHz, DMSO-d₆) δ: 13.00 (br s, 1H), 7.59 (d, J=8.4 Hz, 2H), 7.44-7.40 (m, 3H), 7.35 (d, J=8.4 Hz, 1H), 7.28 (dd, J=2.4 & 8.4 Hz, 1H), 7.21 (d, J=9.6 Hz, 1H), 5.54 (br s, 1H) 4.04 (s, 2H), 2.47-2.41 (m, 2H), 2.35 (s, 3H), 2.33-2.28 (m, 2H), 1.97-1.92 (m, 1H), 1.72-1.65 (m, 1H).

Prep-HPLC condition: Column X-Bridge C-18 (250*19.0 mm) 5μ Buffer MP-(A): 10 Mm ABC in H₂O MP-(B): ACN:WATER (80:20) Gradient (T/% B) 0/5, 15/75, 20/98 Flow Rate 13 mL/min Solubility ACN, WATER, THF

### SYNTHETIC EXAMPLE 72

### Synthesis of Compound II-28

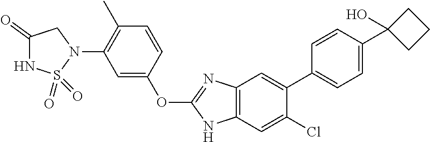 [SMILES: Cc1ccc(Oc2nc3cc(-c4ccc(C5(O)CCC5)cc4)c(Cl)cc3[nH]2)cc1N1CC(=O)NS1(=O)=O]

### Synthesis of 5-(5-((5-bromo-6-chloro-1-((2-(trimethylsilyl)ethoxy)methyl)-1H-benzo[d]imidazol-2-yl)oxy)-2-methylphenyl)-1,2,5-thiadiazolidin-3-one 1,1-dioxide

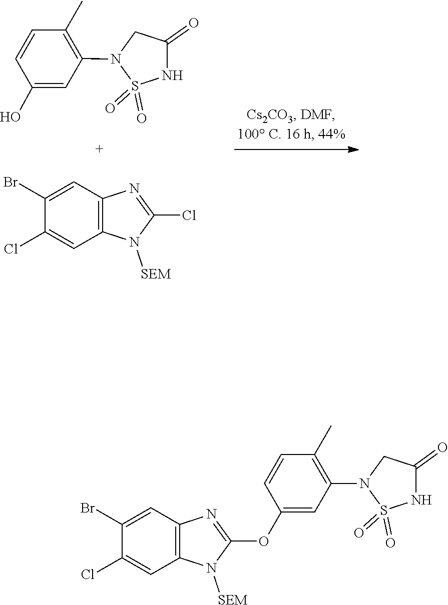

To a stirred solution of 5-bromo-2,6-dichloro-1-((2-(trimethylsilyl)ethoxy)methyl)-1H-benzo[d]imidazole (500 mg, 1.27 mmol, 1.0 eq) in DMF (5 mL, 10 Vol) at room temperature, was added Cs₂CO₃ (1.2 g, 3.81 mmol, 3.0 eq) followed by 5-(5-hydroxy-2-methylphenyl)-1,2,5-thiadiazolidin-3-one 1,1-dioxide (337 mg, 1.39 mmol, 1.1 eq) and stirred at 100° C. for 16 h. After completion of reaction by TLC, the reaction mixture was quenched with ice cold water (50 mL) and extracted with EtOAc (3×30 mL). The combined organic layer was dried over Na₂SO₄, filtered and concentrated under reduced pressure to afford crude product. The crude product was purified by reverse phase column chromatography [elution with 40% ACN in 0.1% FA in H₂O] to afford 5-(5-((5-bromo-6-chloro-1-((2-(trimethylsilyl)ethoxy)methyl)-1H-benzo[d]imidazol-2-yl)oxy)-2-methylphenyl)-1,2,5-thiadiazolidin-3-one 1,1-dioxide as brown solid (340 mg, Yield: 44%). TLC system: MeOH/DCM (10:90), R_{f} value: ˜0.1; LCMS (m/z): 601.27 (M+H)⁺.

### Synthesis of 5-(5-((6-chloro-5-(4-(1-hydroxycyclobutyl)phenyl)-1-((2-(trimethylsilyl)ethoxy)methyl)-1H-benzo[d]imidazol-2-yl)oxy)-2-methylphenyl)-1,2,5-thiadiazolidin-3-one 1,1-dioxide

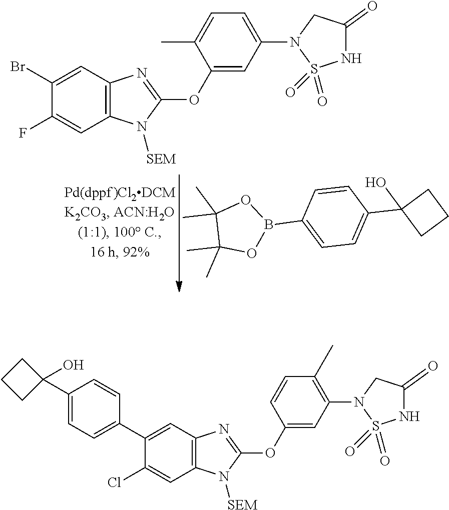 [SMILES: Cc1ccc(N2CC(=O)NS2(=O)=O)cc1Oc1nc2cc(Br)c(F)cc2n1COCC[Si](C)(C)C ; CC1(C)OB(c2ccc(C3(O)CCC3)cc2)OC1(C)C ; Cc1ccc(Oc2nc3cc(-c4ccc(C5(O)CCC5)cc4)c(Cl)cc3n2COCC[Si](C)(C)C)cc1N1CC(=O)NS1(=O)=O]

In a sealed tube, to a degassed solution of 5-(3-((5-bromo-6-chloro-1-((2-(trimethylsilyl)ethoxy)methyl)-1H-benzo[d]imidazol-2-yl)oxy)-4-methylphenyl)-1,2,5-thiadiazolidin-3-one 1,1-dioxide (330 mg, 0.55 mmol, 1.0 eq) and 1-(4-(4,4,5,5-tetramethyl-1,3,2-dioxaborolan-2-yl)phenyl)cyclobutan-1-ol (180 mg, 0.66 mmol, 1.2 eq) in ACN:H₂O (1:1, 10 Vol) at room temperature, was added K₂CO₃ (230 mg, 1.65 mmol, 3.0 eq) followed by Pd(dppf)Cl₂·DCM (45 mg, 0.055 mmol, 0.1 eq) and stirred at 100° C. for 16 h. After completion of reaction by TLC, the reaction mixture was filtered through a pad of diatomaceous earth (e.g., Celite®), washed with EtOAc (50 mL) and filtrate was concentrated under reduced pressure to afford crude product. The crude product was purified by silica gel [60-120] column chromatography [elution with 20% MeOH in DCM] to afford 5-(5-((6-chloro-5-(4-(1-hydroxycyclobutyl)phenyl)-1-((2-(trimethylsilyl)ethoxy)methyl)-1H-benzo[d]imidazol-2-yl)oxy)-2-methylphenyl)-1,2,5-thiadiazolidin-3-one 1,1-dioxide as a brown solid (340 mg, Yield: 92%). TLC system: MeOH/DCM (10:90), R_{f} value: ˜0.2; LCMS (m/z): 669.41 (M+H)⁺; mixture of regioisomers.

### Synthesis of 5-(5-((6-chloro-5-(4-(1-hydroxycyclobutyl)phenyl)-1H-benzo[d]imidazol-2-yl)oxy)-2-methylphenyl)-1,2,5-thiadiazolidin-3-one 1,1-dioxide

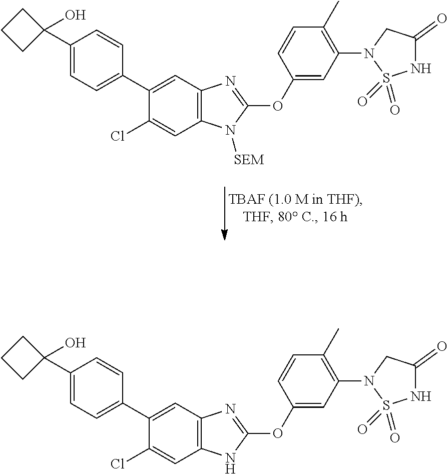 [SMILES: Cc1ccc(Oc2nc3cc(-c4ccc(C5(O)CCC5)cc4)c(Cl)cc3n2COCC[Si](C)(C)C)cc1N1CC(=O)NS1(=O)=O ; Cc1ccc(Oc2nc3cc(-c4ccc(C5(O)CCC5)cc4)c(Cl)cc3[nH]2)cc1N1CC(=O)NS1(=O)=O]

To a stirred solution of 5-(5-((6-chloro-5-(4-(1-hydroxycyclobutyl)phenyl)-1-((2-(trimethylsilyl)ethoxy)methyl)-1H-benzo[d]imidazol-2-yl)oxy)-2-methylphenyl)-1,2,5-thiadiazolidin-3-one 1,1-dioxide (340 mg, 0.51 mmol, 1.0 eq) in THF (10 Vol) at 0° C., was added TBAF (1.0 M in THF) (10 Vol) and stirred at 80° C. for 16 h. After completion of reaction by TLC, the reaction mixture was quenched with NH₄Cl solution (20 mL) and extracted with EtOAc (2×20 mL). The combined organic layer was dried over Na₂SO₄, filtered and concentrated under reduced pressure. The crude was purified by Prep-HPLC, collected fractions were concentrated to afford desired product with ammonium salts. Re-purified by reverse phase column chromatography [elution with 40% ACN in 0.1% FA in H₂O] and lyophilized to afford 5-(5-((6-chloro-5-(4-(1-hydroxycyclobutyl)phenyl)-1H-benzo[d]imidazol-2-yl)oxy)-2-methylphenyl)-1,2,5-thiadiazolidin-3-one 1,1-dioxide as white solid (59 mg, Yield: 21%). TLC system: MeOH/DCM (20:80), R_{f} value: ˜0.1; LCMS (m/z): 539.23 (M+H)⁺; ¹H NMR (400 MHz, DMSO-d₆) δ: 7.56-7.51 (m, 4H), 7.41-7.38 (m, 3H), 7.35-7.33 (m, 2H), 4.27 (s, 2H), 2.47-2.41 (m, 2H), 2.37 (s, 3H), 2.31-2.26 (m, 2H), 1.99-1.89 (m, 1H), 1.72-1.66 (m, 1H).

Prep-HPLC condition: Column YMC C-18 (250*21.2 mm) 5μ Buffer MP-(A): 10 Mm ABC in H₂O MP-(B): ACN:WATER (80:20) Gradient (T/% B) 0/30, 10/65, 25/98 Flow Rate 15 mL/min Solubility ACN, WATER, THF

### BIOLOGICAL EXAMPLE 1

Background: AMPK activators have been designed to treat type 2 diabetes, obesity, and other metabolic diseases, which play a crucial role in modulating AMP-activated protein kinase (AMPK) signaling, coordinating cell growth, autophagy, and metabolism. However, some AMPK activators have cardiotoxicity. To overcome this side effect, the present disclosure includes novel AMPK activators. Compounds of the present disclosure induce specific activation of AMPK in human skeletal muscle, human liver cells and human adipocyte cells rather than cardiomyocyte.

Methods: After starving cells for 3 hours, they were then treated with representative compounds of Structure (I) or (II) for 1 hour. After treatment, cells were analyzed using a glucose uptake assay and a western blot assay for human skeletal myoblasts, human cardiomyocytes cells, human hepatocytes-THLE-2 cells, preadipocyte HWP-c cells.

Results: Compounds I-1, I-7, I-10, and I-11 induced glucose uptake in human skeletal muscle cells, human adipocyte cells, and human hepatocytes-THLE-2 cells. Compared with other components and reference components, Compound I-11 induced glucose uptake in dose-dependent manner. In human cardiomyocyte cells, Compounds I-1, I-7, I-10, and I-11 and PF-06409577 did not induce glucose uptake in dose-dependent manner, however, MK-8722 could increase glucose uptake in HCM.

This data illustrated that compound I-11 and its analogue series could induce glucose uptake in skeletal muscles, adipocytes cells and hepatocytes rather than cardiomyocytes, which increase metabolism in muscle, adipocyte, and hepatocytes, without cardiotoxicity. The western blot data is consistent with glucose uptake data. Without wishing to be bound by theory, it appears that compounds I-1, I-7, I-10, and I-11 could induce p-AMPK, p-ACC, and p-Raptor expression in some high dosage groups. In human liver cells and human adipocyte cells, compound I-11 induced AMPK signaling activation in dose-dependent manner compared with other components, which is consistent with glucose uptake assay. In human cardiomyocyte cells, activation of AMPK signaling pathway was not detected for different dosages.

Conclusion: Compound I-11 and its analogue series can induce AMPK activation in human skeletal muscle myotubes, liver and adipocyte rather than cardiomyocyte, which means these compounds do not have cardiotoxicity.

### BIOLOGICAL EXAMPLE 2

### In Vitro AMPK Kinase Inhibition Experiments

TABLE 2
AMPK kinase binding activity in activation mode
(AC₅₀) screened for 12 AMPK holoenzymes.

| | AMPK | AMPK | AMPK | AMPK |
|---|---|---|---|---|
| Compound | (A2/B1/G3) | (A2/B1/G2) | (A2/B1/G1) | (A2/B2/G3) |
| No. | AC₅₀ (nM) | AC₅₀ (nM) | AC₅₀ (nM) | AC₅₀ (nM) |
| I-1 | +++++ | +++++ | ++++ | − |
| I-2 | ++++ | − | − | − |
| I-3 | ++++ | − | − | − |
| I-4 | ++++ | − | − | − |
| I-9 | ++++ | − | − | − |
| I-10 | ++++ | ++++ | ++++ | − |
| I-12 | ++++ | ++++ | − | − |
| I-13 | +++++ | +++++ | ++++ | − |
| I-14 | ++++ | − | ++++ | − |
| I-15 | ++++ | +++++ | ++++ | − |
| I-16 | +++ | +++ | ++ | − |
| I-17 | +++ | +++ | +++ | − |
| I-18 | − | − | + | − |
| I-20 | +++ | − | ++ | − |
| I-24 | ++++ | − | − | +++ |
| I-25 | ++++ | − | − | +++ |
| I-26 | ++++ | − | − | +++ |
| I-44 | +++ | − | − | − |
| I-45 | ++ | − | − | − |
| I-46 | ++++ | − | − | +++ |
| I-47 | ++++ | − | − | +++ |
| I-48 | ++++ | − | − | +++ |
| I-75 | +++ | − | − | +++ |
| I-76 | +++++ | − | − | +++ |
| II-21 | +++++ | − | − | ++ |
| II-22 | ++++ | − | − | ++++ |
| II-23 | ++++ | − | − | ++++ |
| II-26 | +++++ | − | − | ++++ |

For AMPK activity in Table 2:
+ AC₅₀ concentration ranging from greater than 7500 to 20,000 nM
++ AC₅₀ concentration ranging from greater than 5000 to 7500 nM
+++ AC₅₀ concentration ranging from greater than 500 to 5000 nM
++++ AC₅₀ concentration ranging from greater than 10 to 500 nM
+++++ AC₅₀ concentration of 10 nM or less

### BIOLOGICAL EXAMPLE 3

### Cell Culture and Cell Differentiation

Human skeletal myoblasts were obtained (Thermofisher; BYU) and cultured in DMEM/F12 medium with 10% FBS. For differentiation, 2% horse serum low glucose DMEM medium was employed for 72 hours. Human cardiomyocytes cells (HCM cells; Promocell) were cultured in myocyte growth medium (Promocell). Human preadipocyte HWP-c cells (Promocell) were obtained from upper arm of female diabetic II donor. Human preadipocyte HWP-c cells (Promocell) were obtained from abdomen of female diabetic II donor. For differentiating human preadipocytes, cells were cultured in preadipocyte growth medium to total confluency stage. The growth medium was replaced with preadipocyte differentiation medium for 72 hours, then changed to PromoCell adipocyte nutrition medium to complete the differentiation process. THLE-2 cells were cultured in BEGM from Lonza/Clonetics Corporation, Walkersville, MD 21793 (BEGM Bullet Kit; CC3170). HepG2, L6 and C2Cl2 cells were cultured in EMEM medium.

### BIOLOGICAL EXAMPLE 4

### Glucose Uptake Assay

After differentiation myoblasts, preadipocyte cells were starved by removing the medium and adding 100 μL DMEM without serum. The medium was then replaced with 100 μL DMEM±1 μM insulin at indicated concentration of representative compounds without serum or glucose. The cells were then incubated for 1 hour at 37° C. in 5% CO₂. The cells were then removed from the medium and 50 μL of 0.1 mM 2DG in PBS was added. Cells were incubated for 30 minutes at 25° C. 25 μL of Stop Buffer was added and samples were mixed. 25 μL of Neutralization Buffer was then added and mixed. 100 μL of 2DG6P Detection Reagent was then added and mixed. Samples were then incubated for 1 hour at 25° C. Luminescence was then recorded with 0.3-1 second integration on a luminometer.

### BIOLOGICAL EXAMPLE 5

### Western Blot Assay

Samples for immunoblotting studies were prepared for electrophoresis by adding in a final concentration of 1× Laemmli loading buffer (supplemented with 5% 2-mercaptoethanol). Samples were then electrophorized by SDS-PAGE (4-15% or 4-20% Criterion TGX Precast gel, Bio-Rad) at a constant voltage within the range of 140V. Proteins were transferred to a nitrocellulose membrane (Bio-Rad) using the Trans-Blot Turbo transfer system (Bio-Rad). Membranes were blocked with 5% BSA in TBST for a minimum of 1 hour at room temperature before incubating with the following primary antibodies:
Phospho-AMPKα (Thr172) (40H9) Rabbit mAb (CST, cat no: 2535S),
AMPKα Antibody (CST, cat no: 2532S)
Phospho-Raptor (Ser792) Antibody (CST, cat no: 2083S)
Raptor (24C12) Rabbit mAb (CST, cat no: 2280S)
Phospho-Acetyl-CoA Carboxylase (Ser79) Antibody (CST, cat no: 3661S)
Acetyl-CoA Carboxylase Antibody (CST, cat no: 3662S)
AMPKα2 Antibody (CST, cat no: 2757S)
AMPKβ1 antibody (CST, cat no: 12063)
AMPKβ2 Antibody (CST, CST, cat no: 4148S)
AMPKγ3 Antibody (CST, cat no: 2550S).

### BIOLOGICAL EXAMPLE 6

### PK Oral Bioavailability Experiment

The bioavailability, pharmacokinetics, and brain to plasma distribution in mouse plasma following oral and intravenous administration of compound I-11 in male Swiss Albino Mouse was performed. A total of 24 male Swiss albino mouse were allotted for the study, Animals were allotted to 2 groups containing 12 mice/group, on day of dosing all the animals were fasted 3 hours before dosing. The test formulation was administered as a single dose by intravenous and oral route via tail vein and oral gavage needle at a dose of 1 and 5 mg/kg with a dose volume of 5 and 10 mL/kg, respectively.

Approximately 0.1 mL of blood sample from each animal was collected via retro orbital at predetermined time points (3 animals/Timepoint) in pre-labeled Eppendorf tubes containing anticoagulant. After blood collection at 0.5 h and 1 h, after collection of CSF, whole blood was withdrawn, and brain tissue was collected as a terminal sampling. Blood samples were centrifuged at 10000 rpm for 5 min under refrigeration (2-4° C.) within 0.5 h to obtain plasma. The separated plasma samples were collected in pre labeled tubes within 60 min of separation and stored at −20° C. up to 24 h. Post 24 h sample collection, all plasma samples were transferred to deep freezer custodian to store below or at −70° C. until shipment.

Similarly, CSF and Brain were collected at 0.5 h and 1h time point by scarification. Tissues were blotted and dried and then transferred to 15 mL falcon tubes. Initially all tissue samples were stored at −20±3° C. Later all brain tissue samples were homogenized with PBS at 1:4 ratio and the brain homogenate were transferred to deep freezer (−70±10° C.) and stored.

Mouse plasma, CSF and Brain homogenate concentrations were analyzed to quantify Compound I-11 using a fit-for purpose LC-MS/MS method with LLOQ of 0.977 ng/mL. The PK parameters were evaluated using Phoenix WinNonlin® Ent-Version 8.3 by non-compartmental analysis.

Results: Compound I-11 attained moderate systemic exposure with low clearance. Compound I-11 demonstrated biphasic elimination. Later time points, i.e., after 4 h plasma concentration was increasing than previously reported concentrations it could be one of the reasons of enterohepatic circulation or Pgp inhibitor further studies may require evaluating this second peak phenomenon.

### BIOLOGICAL EXAMPLE 7

### PK Oral Bioavailability Experiment

The bioavailability, pharmacokinetics and CSF and Brain uptake ratio of Compound I-11 following intravenous bolus and oral administration in male Wistar rat at a dose of 1 mg/kg and 5 mg/kg was performed. A total of 18 male Wistar rats were allotted for the study. Animals were allotted in to 2 groups of 9 rat per each group. In each group, first set of three animals were used for PK study, next set of three and followed by other set of three animals were used for 0.5 h and 1 h brain, CSF, and blood collection purpose for intravenous and oral groups, respectively. On day of dosing all the animals were fasted overnight. The compound I-11 formulation was administered to G1 and G2 as a single dose by intravenous at dose of 1 mg/kg and oral route at a dose of 5 mg/kg. Compound I-11 was administered with a dose volume of 2 & 10 mL/kg for intravenous and oral routes, respectively.

Approximately 0.3 mL of blood sample from each animal was collected via jugular vein at predetermined time points at 0.083 (only IV), 0.25, 0.5, 1, 2, 4, 6, 8 and 24 h intervals in pre-labeled Eppendorf tubes containing anticoagulant K₂EDTA (20 μL per mL of blood). Blood sample was centrifuged at 5000 rpm for 10 min under refrigeration (2-4° C.) to obtain plasma. For brain and CSF uptake study, after 0.5 h and 1 h CSF and whole brain was withdrawn, and brain tissue was homogenized as a terminal sampling. Blood samples were centrifuged under refrigeration (2-4° C.) within 0.5 h to obtain plasma. The separated plasma samples were collected in pre labeled tubes within 60 min of separation and stored at −20° C. up to 24 h. Post 24 h sample collection, all plasma samples were transferred to deep freezer custodian to store below or at −70° C. till shipment.

Brain tissues were blotted and dried and then transferred to 15 mL falcon tubes. Initially all tissue samples were stored at −20±3° C. Later, all brain tissue samples were homogenized with PBS at 1:4 ratio and the Brain homogenate was transferred to Deep freezer (−70±10° C.) and stored until bioanalysis.

Plasma, CSF and Brain homogenate concentrations were analyzed to quantify compound I-11 using a fit-for purpose LC-MS/MS method with LLOQ of 1 ng/mL. The PK parameters were evaluated using Phoenix WinNonlin® Ent-Version 8.3 by non-compartmental analysis.

Results: Compound I-11 attained low systemic exposure following oral administration. due to the liver first pass effect. Oral bioavailability was found to be 6%. Compound I-11 is not a lipophilic in nature as there was no concentrations in brain tissue.

### BIOLOGICAL EXAMPLE 8

### PK Oral Bioavailability Experiment

The oral bioavailability and pharmacokinetic parameters of compound I-11 in dog plasma following oral and intravenous administration in male Beagle dogs at 5 mg/kg and 1 mg/kg was performed. A total of 3 animals were segregated in to one group and each animal received oral dose followed by intravenous infusion (15 minutes) as a cross over design with 7 days of no dose interval between periods. Animals in period 1, received oral dose of 5 mg/kg of compound I-11 with a dose volume of 5 mL/kg using oral gavage tube, following oral dosing, around 2 mL of water was administered to each dog via gavage tube to flush the dead volume. Similarly, in period 2 after 7 days of no dose interval, animals were received intravenous infusion for 15 min at a dose of 1 mg/kg via saphenous vein at a dose volume of 1 mL/kg, each dog was observed after administration of the test item for presence or absence of clinical signs.

A total of 11 time points for oral and 12 blood samples for IV per each animal were collected, approximately 1 mL were collected from each dog in pre-labeled Eppendorf tubes containing 20 μL/mL of 200 mM K₂ EDTA. Blood was withdrawn at scheduled time points via cephalic vein in pre-labelled Eppendorf tubes. The pre-dose blood sample was collected before to drug administration. Post dose samples were collected within stipulated time. The blood samples were kept on crushed ice to maintain the temperature until centrifugation. Plasma was separated and stored under −70° C. Dog plasma concentrations were analyzed to quantify compound I-11 using a fit-for purpose LC-MS/MS method with LLOQ of 1.020 ng/mL. The PK parameters were evaluated using Phoenix WinNonlin® Ent-Version 8.3 by non-compartmental analysis.

Results: Oral administration of compound I-11 exhibited low systemic exposure and oral bioavailability was found to low (5.27±2.94) compared with intravenous infusion (1 mg/kg). This could be a possible reason of the hepatic first pass metabolism.

BIOLOGICAL EXAMPLE 9
IN VIVO EFFICACY DIABETES MODEL

| | | Dose; Route of | |
|---|---|---|---|
| | | administration; | |
| | | Dosage volume | Concentration |
| Group | Treatment | (10 mL/kg), Duration | mg/mL |
| G1 | Vehicle | 10 mL/kg; p.o.; QD | 0 |
| | Control | | |
| G2 | Compound | 30 mg/kg, p.o.; QD | 3 |
| | I-11 | | |
| G3 | Compound | 50 mg/kg, p.o.; QD | 5 |
| | I-11 | | |

The doses of the test item to be administered were calculated daily in mg/kg based on the latest bodyweight of the animal. The dose volume was calculated based on mg/kg of body weight and adjusted with each bodyweight collection. Dose and route of the test compounds administration are selected as per the supporting literature.

Male db/db mice were procured from The Jackson Laboratory. All the animals were allowed to quarantine for 7 days and acclimatize for one day prior to experiment initiation. During this period, mice were observed daily once for clinical signs. On Day 1, Animals were randomized based on body weight and blood glucose levels (Accu-Chek) into three groups. G1-Group were administered with vehicle (10 mL/kg, p.o., Q.D.), G2-Group were administered with compound I-11 (30 mg/kg, p.o., Q.D.) and G3-Group were administered with compound I-11 (50 mg/kg, p.o., Q.D.) for 36 days. During the study period the body weight of mice was recorded weekly thrice, and clinical scoring was done on daily basis. The OGTT was performed on day 21. On day 36 blood was collected and centrifuged at 4° C., 10,000 RPM for 10 minutes for plasma collection. The plasma stored at −80° C. for estimation of plasma HDL and LDL. All the animals were humanely sacrificed by CO₂. At the time of necropsy liver, adipose tissue and pancreas weight were taken. The liver and pancreas were stored at −80° C.

Compound I-11 showed a dose-dependent decrease in fasting blood glucose levels as compared with vehicle control animals on day 21, 28, and 36. Compound I-11 (30 & 50 mg/kg, p.o., QD) showed a significant decrease in fasting blood glucose levels as compared with vehicle control animals on day 21 and 28. Compound I-11 (50 mg/kg, p.o., QD) showed a significant decrease in fasting blood glucose levels as compared with vehicle control animals on day 36.

### BIOLOGICAL EXAMPLE 10

### In Vivo Efficacy Obesity Model

Upon arrival, 18-22-week-old animals are group-housed and given 60% high-fat diet (Research diets D12492). Animals and food are weighed as the animals are put into cages. Animals and food are weighed again on study days −13, −10, −7, −5, and −3 to confirm that animals are eating and gaining body weight on the 60% high-fat diet. Animal weight and food consumption are measured 3 times a week throughout the study, beginning on day 0. On study day −3, animals are randomized into groups by body weight and composition. After randomization, treatment began.

The animals fast for 4 hours on study days −4 and 18. The necropsy is done on day 28. Animals are humanely euthanized with inhaled isoflurane anesthesia, bled to exsanguination, and then undergo cervical dislocation to confirm euthanasia. Necropsy sample and data collection are specified in the Necropsy Information section.

| | | | | | Dose |
|---|---|---|---|---|---|
| | | ²Dose | | Dose Vol | Conc |
| Group* | Treatment | mg/kg | Regimen | (mL/kg)³ | (mg/ml) |
| 1 | Vehicle | 0 | QD, Days | 10 | 0 |
| | | | 0-28 | | |
| 2 | Compound | 30 | QD, Days | 10 | 3 |
| | I-11 | | 0-28 | | |
| 3 | Compound | 50 | QD, Days | 10 | 5 |
| | I-11 | | 0-28 | | |

*for all groups n = 10, treatment was 60% HFD, and dose was administered via PO route

The doses of the test item to be administered are calculated daily in mg/kg based on the latest bodyweight of the animal. The dose volume is calculated based on mL/kg of body weight and adjusted with each bodyweight collection. Animals in HFD diet groups arrived pre-conditioned on HFD research diets.

### BIOLOGICAL EXAMPLE 11

### Obesity Study Parameters and Results

Compound I-11 was formulated as a suspension in DMSO (2%), Cremophor EL (2%), Tween (1%) 0.5% methyl cellulose (35%), and pure water (60%). Doses were 30 and 50 mg/kg and administered orally (p.o.) once a day (QD) for 36 days. Dose volume was 10 mL/kg, which was administered to male, 18-22-week-old mice (Mus musculus—C57BL-6) with body weight between 18-19.4 g each. Mice were grouped into 3 groups of 10. The diet for induced obesity mice were selected as a test system

Mice were randomized based on glucose levels and body weight. After randomization, extra animals were used for blank sample preparation. Details of study groups and treatments are summarized in the table below.

| | | Dosage | |
|---|---|---|---|
| Groups | Treatment | Information | Animals/Group |
| G1 | Disease Control + HFD | — | 10 |
| G2 | Compound I-11 + HFD | 30 mg/kg | 10 |
| G3 | Compound I-11 + HFD | 50 mg/kg | 10 |

All dosages were oral for 36 days and a dose volume of 10 mL/kg

Animals and food were weighed as the animals are put into cages. Animals and food were weighed again on study days −13, −10, −7, −5, and −3 to confirm that animals were eating and gaining body weight on the 60% high-fat diet. Animal weight and food consumption was measured 3 times a week throughout the study, beginning on day 0.

On study day −3, animals were randomized into groups by body weight and composition (DEXA). During this period, mice were observed daily once for clinical signs.

Body composition was tested on day −3 and on study day 25. During the study period body weight of mice was recorded weekly thrice and clinical scoring was done on daily basis.

Euthanasia and necropsy were done on day 36. Animals fasted 12 hours followed by blood glucose monitoring and plasma collection. Plasma stored at −80° C. for estimation of plasma glucose and plasma insulin levels. The whole liver, gastrocnemius, subcutaneous adipose, epididymal white adipose tissue weight of each mouse were recorded and calculated absolute and relative weight at the study day sacrifice.

Animals were observed daily for any clinical signs to treatment throughout the experiment. Cage-side observations were made to detect any changes and general activity of the animals. After dose administration all the mice were observed carefully for treatment related clinical signs, including morbidity and mortality, once a day. Body weight of each mouse was measured weekly three times during the study period. Fed blood glucose levels were monitored on days 14, 21, and 34.

Plasma lipid profile: On day 34 total cholesterol levels, triglyceride, HDL, and LDL levels were detected. Plasma insulin (fasting) and plasma NEFA (fasting) was tested on day 36. Cumulative food consumption was measured daily starting from day 1 onwards up to end of the study period. Liver, heart, jejunum, and brain tissue were collected for additional testing.

All data is expressed as the mean±SD unless indicated otherwise. Data on each parameter is summarized in tabular form. Appropriate graphical representation was done using suitable method. Statistical analysis was done with GraphPad Prism-5 using one-way ANOVA followed by Dunnett's test/Tukey's post hoc test. Data were considered statistically significant if P value is less than 0.05.

Results There were no observations of clinical signs or morbidity, and no mortality was reported during the study. The effect of compound I-11 on body weight in high fat diet mice is shown in the table below (for day 36). In brief, G2 and G3 did not show significant change in body weight (g) as compared to G1 up to day 36.

| Treatment Groups | Body Weight |
|---|---|
| G1 | 48.24 ± 1.24 |
| G2 | 50.20 ± 1.01 |
| G3 | 49.37 ± 0.89 |

The percentage change in body weight for this study is shown in the table below. As noted above, G2 and G3 did not show significant change in body weight (g) as compared to G1 up to day 36.

| Treatment Groups | % Body |
|---|---|
| G1 | −2.86 ± 2.24 |
| G2 | 1.40 ± 0.21 |
| G3 | −0.34 ± 0.73 |

The effect of compound I-11 on cumulative feed intake in high fat diet mice is shown in the table below up to day 35. In sum, G2 and G3 did not show significant change in feed intake (g) as compared to G1 up to day 35.

| Treatment Groups | Feed Intake (g) |
|---|---|
| G1 | 299.3 ± 24.2 |
| G2 | 308.3 ± 33.5 |
| G3 | 313.3 ± 18.5 |

The effect of compound I-11 on fed blood glucose in high fat diet mice (mg/dL) is shown in the table below for day 14. G2 showed significant decrease on day 14 and no significant on day 21 and 34 in fed blood glucose levels as compared to G1. G3 did not show decrease in fed blood glucose levels as compared to G1 on day 14,21 and 34.

| Treatment | Fed Blood Glucose |
|---|---|
| Groups | (mg/dL) |
| G1 | 177.00 ± 8.64 |
| G2 | 155.30 ± 5.10* |
| G3 | 160.70 ± 2.34 |

The results for fed blood glucose in high fat diet mice (mg/dL) is shown in the table below for day 21 are shown below.

| Treatment | Fed Blood Glucose |
|---|---|
| Groups | (mg/dL) |
| G1 | 134.10 ± 4.51 |
| G2 | 146.60 ± 5.74 |
| G3 | 145.40 ± 6.01 |

The results for fed blood glucose in high fat diet mice (mg/dL) is shown in the table below for day 34 are shown below.

| Treatment | Fed Blood Glucose |
|---|---|
| Groups | (mg/dL) |
| G1 | 158.60 ± 5.50 |
| G2 | 164.80 ± 3.84 |
| G3 | 158.00 ± 5.80 |

The effect of compound I-11 on liver weight (g) in high fat diet mice is shown for 10 day 36 in the two tables below (absolute and relative, respectively). G2 and G3 did not show significant change in absolute and relative liver weight (g) as compared to G1 mice on day 36.

### Absolute Liver Weight:

| Treatment | Absolute Liver |
|---|---|
| Groups | Weight (g) |
| G1 | 2.124 ± 0.117 |
| G2 | 2.042 ± 0.147 |
| G3 | 2.056 ± 0.092 |

### Relative Liver Weight:

| Treatment | Relative Liver |
|---|---|
| Groups | Weight (g) |
| G1 | 0.044 ± 0.002 |
| G2 | 0.040 ± 0.020 |
| G3 | 0.042 ± 0.002 |

The effect of compound I-11 on epididymal fat (g) in high fat diet mice is shown in the two tables below (absolute and relative, respectively). G2 and G3 did not show significant change in absolute and relative epidermal fat (g) as compared to G1 mice on day 36.

### Absolute Epididymal Fat:

| Treatment | Absolute Epididymal |
|---|---|
| Groups | Fat (g) |
| G1 | 1.514 ± 0.095 |
| G2 | 1.399 ± 0.061 |
| G3 | 1.392 ± 0.061 |

### Relative Epididymal Fat:

| Treatment | Relative Epididymal |
|---|---|
| Groups | Weight (g) |
| G1 | 0.032 ± 0.003 |
| G2 | 0.028 ± 0.001 |
| G3 | 0.028 ± 0.001 |

The effect of compound I-11 on abdominal fat (g) in high fat diet mice is shown for day 36 in the two tables below (absolute and relative, respectively). G2 and G3 did not show significant change in absolute and relative peripheral fat (g) as compared to G1 mice on day 36.

### Absolute Abdominal:

| Treatment | Absolute Abdominal |
|---|---|
| Groups | Fat Weight (g) |
| G1 | 2.285 ± 0.178 |
| G2 | 2.447 ± 0.171 |
| G3 | 2.541 ± 0.090 |

### Relative Abdominal Fat:

| Treatment | Relative Abdominal |
|---|---|
| Groups | Fat Weight (g) |
| G1 | 0.047 ± 0.003 |
| G2 | 0.049 ± 0.001 |
| G3 | 0.052 ± 0.002 |

The effect of compound I-11 on fasting glucose and total cholesterol (mg/dL) in high fat diet mice on day 36 is shown in the two tables below, respectively. G2 and G3 did not show significant change in glucose level (mg/dL) and total cholesterol (mg/dL) as compared to G1 mice on day 36.

| Treatment | Fasting Glucose |
|---|---|
| Groups | (mg/dL) |
| G1 | 172.21 ± 25.14 |
| G2 | 157.22 ± 19.88 |
| G3 | 224.94 ± 30.90 |

| Treatment | Fasting Total |
|---|---|
| Groups | Cholesterol (mg/dL) |
| G1 | 195.43 ± 15.95 |
| G2 | 228.91 ± 22.85 |
| G3 | 227.32 ± 22.35 |

The effect of compound I-11 on fasting HDL (mg/dL) and LDL (mg/dL) in high fat diet mice for day 36 is shown in the two tables below, respectively. G2 and G3 did not show significant change in HDL (mg/dL) and LDL (mg/dL) as compared to G1 mice on day 36.

| Treatment | Fasting HDL |
|---|---|
| Groups | (mg/dL) |
| G1 | 138.00 ± 10.98 |
| G2 | 148.59 ± 24.76 |
| G3 | 184.23 ± 19.22 |

| Treatment | Fasting LDL |
|---|---|
| Groups | (mg/dL) |
| G1 | 24.18 ± 2.48 |
| G2 | 23.46 ± 2.13 |
| G3 | 25.59 ± 1.76 |

The effect of compound I-11 on fasting triglycerides (mg/dL) in high fat diet mice on day 36 is shown in the table below. G2 and G3 did not show significant change in triglycerides (mg/dL) as compared to G1 mice on day 36.

| Treatment | Fasting HDL |
|---|---|
| Groups | (mg/dL) |
| G1 | 74.38 ± 6.04 |
| G2 | 86.81 ± 6.81 |
| G3 | 78.83 ± 6.75 |

The effect of compound I-11 on lean+bone mineral content (BMC-gm) in high fat diet mice on days −3, 14, and 34 is shown in the table below. G2 showed significant change on day 34 as compared to G1 control mice. G3 showed a trend of increase as compared to G1 mice, but the increase was not significant.

| Treatment | | | |
|---|---|---|---|
| Groups | Day −3 | Day 14 | Day - 34 |
| G1 | 27.11 ± 0.70 | 24.59 ± 0.61 | 22.71 ± 0.48 |
| G2 | 26.78 ± 0.50 | 24.11 ± 0.71 | 19.70 ± 0.98 |
| G3 | 26.97 ± 1.03 | 25.83 ± 0.49 | 22.16 ± 0.92 |

The effect of compound I-11 on total mass (gm) in high fat diet mice is shown for days −3, 14, and 34 in the table below. G2 and G3 did not show significant change in total mass as compared to G1 mice on day 34.

| Treatment | | | |
|---|---|---|---|
| Groups | Day −3 | Day 14 | Day 34 |
| G1 | 50.75 ± 0.89 | 52.43 ± 1.01 | 53.91 ± 1.13 |
| G2 | 49.96 ± 0.75 | 52.05 ± 1.04 | 54.86 ± 1.18 |
| G3 | 50.40 ± 0.80 | 51.78 ± 0.85 | 53.35 ± 1.02 |

The effect of compound I-11 on area (cm²; DEXA scan) in high fat diet mice is shown for days −3, 14 and 34 in the table below. G2 and G3 did not show significant change as compared to G1 on day 34.

| Treatment | | | |
|---|---|---|---|
| Groups | Day −3 | Day 14 | Day 34 |
| G1 | 12.77 ± 0.32 | 13.52 ± 0.17 | 13.82 ± 0.26 |
| G2 | 12.61 ± 0.23 | 13.26 ± 0.24 | 13.90 ± 0.20 |
| G3 | 12.56 ± 0.30 | 12.93 ± 0.23 | 13.89 ± 0.29 |

The effect of compound I-11 on bone mineral density (BMD) and bone mineral content (BMC) in high fat diet mice for days −3, 14, and 34 are shown in the tables below, respectively. G2 and G3 did not show significant change in BMC and BMD as compared to G1 on day 34

| Treatment | | | |
|---|---|---|---|
| Groups | Day −3 | Day 14 | Day 34 |
| G1 | 0.08 ± 0.00 | 0.08 ± 0.00 | 0.08 ± 0.00 |
| G2 | 0.08 ± 0.00 | 0.08 ± 0.00 | 0.08 ± 0.00 |
| G3 | 0.08 ± 0.00 | 0.08 ± 0.00 | 0.08 ± 0.00 |

| Treatment | | | |
|---|---|---|---|
| Groups | Day −3 | Day 14 | Day 34 |
| G1 | 1.01 ± 0.02 | 1.07 ± 0.01 | 1.12 ± 0.02 |
| G2 | 1.01 ± 0.01 | 1.04 ± 0.02 | 1.11 ± 0.02 |
| G3 | 1.01 ± 0.02 | 1.04 ± 0.02 | 1.12 ± 0.02 |

The effect of compound I-11 on % fat mass in high fat diet mice on days −3, 14, and 34 is shown in the table below. G2 showed significant increase in % fat mass as compared to G1 on day 34. G3 did not show significant change in % fat mass as compared to G1

| Treatment | | | |
|---|---|---|---|
| Groups | Day −3 | Day 14 | Day 34 |
| G1 | 46.38 ± 1.85 | 53.02 ± 1.15 | 57.79 ± 0.73 |
| G2 | 46.39 ± 0.83 | 53.75 ± 0.88 | 64.20 ± 1.33 |
| G3 | 46.37 ± 2.15 | 50.05 ± 1.02 | 58.56 ± 1.26 |

The effect of compound I-11 on fat mass (gm) in high fat diet mice is shown in the table below. G2 showed significant change in fat mass as compared to G1 mice on day 34. G3 did not show significant change in fat mass compared to G1.

| Treatment | | | |
|---|---|---|---|
| Groups | Day −3 | Day 14 | Day 34 |
| G1 | 23.64 ± 1.34 | 27.83 ± 0.93 | 31.19 ± 0.88 |
| G2 | 23.20 ± 0.60 | 27.97 ± 0.67 | 35.16 ± 0.82 |
| G3 | 23.44 ± 1.29 | 25.95 ± 0.84 | 31.19 ± 0.70 |

The effect of compound I-11 on plasma and tissue concentration for day 36 is shown in the table below. Compound I-11 at doses of 30 and 50 mg/kg showed exposure in plasma, liver, and adipose tissue whereas less exposure in jejunum, heart, and skeletal muscle. Note: in jejunum at 1 hour 1 mouse in each group showed maximum exposure.

| | | | | | Adipose tissue |
|---|---|---|---|---|---|
| Treatment | Time | Plasma | Brain | Liver | (abdominal fat) |
| Group | point | (ng/mL) | (ng/mL) | (ng/mL) | (ng/mL) |
| G2 | 1 hr | 627 | BLQ | 118 | 57 |
| | 3 hr | 147 | BLQ | 74 | 23 |
| | 4 hr | 121 | 6 | 106 | 38 |
| G3 | 1 hr | 501 | BLQ | 80 | 2003 |
| | 3 hr | 199 | BLQ | 64 | 1588 |
| | 4 hr | 112 | BLQ | 71 | 1765 |

| Treatment | Time | Jejunum | Heart | Skeletal muscle |
|---|---|---|---|---|
| Group | point | (ng/mL) | (ng/mL) | (ng/mL) |
| G2 | 1 hr | 123642 | 103 | 287 |
| | 3 hr | 35 | 32 | BLQ |
| | 4 hr | 27 | 31 | 56 |
| G3 | 1 hr | 4386 | 52 | BLQ |
| | 3 hr | 165 | 36 | BLQ |
| | 4 hr | 55 | 102 | BLQ |

Compound I-11 did not show any significant change in plasma NEFA levels compared to vehicle control group. The effect of compound I-11 in plasma non-esterified Fatty Acids (mmol/L) in high fat diet mice is shown in the table below.

| Treatment Groups | Day 36 |
|---|---|
| G1 | 1.5 ± 0.1 |
| G2 | 1.6 ± 0.2 |
| G3 | 1.6 ± 0.1 |

### BIOLOGICAL EXAMPLE 12

### Evaluating Compound Effect in Type 2 Diabetes

The objective of this study was to evaluate the effect of compound I-11 in db/db mouse model of progressive type 2 diabetes. A mixture of DMSO (2%), Cremophor EL (2%), Tween 80 (1%), 0.5% methyl cellulose (35%), and water (60%) was used for the formulation. Doses were prepared at concentrations of 30 and 50 mg/kg. Administration was oral, daily for 36 days. Male mice (Mus musculus; db/db C57BL/6) aged 8-9 weeks and weighing less than or equal to 40 grams were used for the study. Groups were assigned 10 mice per group.

| Groups | Treatment | Dose (10 mL/kg) |
|---|---|---|
| G1 | Vehicle Control | 10 mL/kg |
| G2 | Compound I-11 | 30 mg/kg |
| G3 | Compound I-11 | 50 mg/kg |

During the study period body weight of mice were recorded weekly thrice and clinical scoring were done on daily basis. The OGTT was performed on day 21. On day 36 blood was collected and centrifuged at 4° C., 10000 RPM for 10 minutes for plasma collection. The plasma stored at −80° C. for estimation of plasma HDL and LDL. All the animals were humanely sacrificed by CO₂. At the time of necropsy liver, adipose tissue and pancreas weight were taken. Liver and pancreas were stored at −80° C.

Animals were observed daily for any clinical signs to treatment throughout the experiment. Cage-side observations were made to detect any changes and general activity of the animals. After dose administration all the mice were observed carefully for treatment related clinical signs, including morbidity and mortality, once a day.

Body weight of each mouse was recorded at the time of randomization and thrice a week during study period. OGTT were done at day 21. On day 20 mice were fasted over night and on day 21 mice were treated with respective treatment-60 minute prior to glucose load 1 gram/kg. Plasma glucose was measured at 0 min (before glucose load), 15 min, 30 min, 60 min, 90 min and 120 min.

Glucose estimation in urine was done at terminal point on day 32. The urine of individual mice was collected by placing the mice in metabolic cages. Urine glucose levels were measured by calorimetric method in collected urine.

Plasma lipid profile: Triglyceride, HDL and LDL were performed at the end of the study by appropriate kit or using Fully Auto Biochemistry Analyzer Selectra PRO M Lite. Fat pad weight was recorded at terminal point. Fructosamine were measured by ELISA kit (Biomol, G-MOEB2562.96).

All data is expressed as mean±SEM unless otherwise noted. Statistical analysis was done with GraphPad Prism-5 using one-way ANOVA followed by Dunnett's test/Tukey's post hoc test. Data were considered statistically significant if P value is less than 0.05.

There were no observations of clinical signs or morbidity, and no mortality was reported during the course of the study. The effect of compound I-11 on db/db body weight is shown in the table below. Mice does with Compound I-11did not show significant decrease in body weight as compared with vehicle control animals at both 30 and 50 mg/kg doses.

| Treatment Groups | Body Weight (g) |
|---|---|
| G1 | 41.24 ± 0.79 |
| G2 | 42.17 ± 0.67 |
| G3 | 42.06 ± 0.67 |

The effect of compound I-11 on fed plasma glucose at day 14 and day 29 is shown in the table below. G2 and G3 did not show a decrease in fed plasma glucose levels vs. vehicle control group on day 14 and day 29.

Day 14:

| Treatment Groups | Fed Plasma Glucose (mg/dL) |
|---|---|
| G1 | 589.32 ± 25.04 |
| G2 | 585.32 ± 15.82 |
| G3 | 550.02 ± 26.95 |

Day 29:

| Treatment Groups | Fed Plasma Glucose (mg/dL) |
|---|---|
| G1 | 530.99 ± 23.47 |
| G2 | 527.86 ± 27.75 |
| G3 | 517.80 ± 25.81 |

The effect of compound I-11 in db/db mice on fasting blood glucose at day 21, 28 and 36 (˜12 hr) is shown in the tables below. Compound I-11 showed a dose-dependent decrease in fasting blood glucose levels as compare with vehicle control animals on day 21, 28, and 36.

G2 and G3 each showed a significant decrease in fasting blood glucose levels as compared with vehicle control animals on day 21 and 28. G3 showed significant decrease in fasting blood glucose levels as compared with vehicle control animals on day 36.

Day 21:

| Treatment Groups | Fasting Blood Glucose (~12 hrs) (mg/dL)) |
|---|---|
| G1 | 369.70 ± 28.51 |
| G2 | 263.60 ± 21.76** |
| G3 | 191.80 ± 15.79**** |

Day 28:

| Treatment Groups | Fasting Blood Glucose (~12 hrs) (mg/dL) |
|---|---|
| G1 | 371.30 ± 26.18 |
| G2 | 313.50 ± 14.93* |
| G3 | 260.30 ± 14.11*** |

Day 36:

| Treatment Groups | Fasting Blood Glucose (~12 hrs) (mg/dL) |
|---|---|
| G1 | 375.29 ± 15.74 |
| G2 | 335.70 ± 16.88 |
| G3 | 316.40 ± 9.81* |

The effect of compound I-11 on oral glucose tolerance test is shown in the table below. G2 showed decrease (7.5%) in the plasma glucose compared to vehicle control however statistically not significant. G3 showed significant decrease (14%) in the plasma glucose compared to vehicle control.

| Treatment | Fasting Plasma | 15 min Plasma | 30 min Plasma |
|---|---|---|---|
| Groups | Glucose (mg/dL) | Glucose (mg/dL) | Glucose (mg/dL) |
| G1 | 354.59 ± 27.77 | 724.10 ± 27.36 | 642.29 ± 23.80 |
| G2 | 287.70 ± 34.54 | 676.20 ± 19.94 | 619.54 ± 26.68 |
| G3 | 204.64 ± 29.60 | 670.72 ± 23.88 | 560.85 ± 37.42 |

| Treatment | 60 min Plasma | 120 min Plasma |
|---|---|---|
| Groups | Glucose (mg/dL) | Glucose (mg/dL) |
| G1 | 567.47 ± 36.63 | 436.91 ± 14.69 |
| G2 | 520.87 ± 46.63 | 397.78 ± 9.65 |
| G3 | 485.98 ± 32.31 | 378.40 ± 18.24 |

Administration of compound I-11 resulted in dose dependent decreases in glucose as measured by area under the curve (AUC). G2 showed (7.5%) decrease in the plasma glucose compared to vehicle control however statistically not significant. G3 showed significant decrease (14%) in the plasma glucose compared to vehicle control.

| Treatment Groups | mg/dL*min |
|---|---|
| G1 | 66616.20 ± 1791.29 |
| G2 | 61613.10 ± 2572.98 |
| G3 | 57436.00 ± 2283.79 |

G3 showed significant decrease in fasting urine glucose levels as compared with vehicle control animals.

| Treatment Groups | Urine Glucose (mg/dL) |
|---|---|
| G1 | 1659.98 ± 15.14 |
| G2 | 1694.43 ± 10.56 |
| G3 | 1605.11 ± 15.29* |

G3 showed decrease in plasma cholesterol and plasma triglyceride levels compared to G1-Vehicle Control (10 mL/kg), p.o.; Q.D. however it was nonsignificant.

| Treatment Groups | Plasma Cholesterol (mg/dL) |
|---|---|
| G1 | 114.37 ± 12.52 |
| G2 | 118.13 ± 8.39 |
| G3 | 97.30 ± 8.76 |

| Treatment Groups | Plasma Triglyceride (mg/dL) |
|---|---|
| G1 | 128.58 ± 16.86 |
| G2 | 156.87 ± 17.41 |
| G3 | 97.55 ± 11.60 |

G3 showed decrease in plasma HDL and plasma LDL levels, however the effect was nonsignificant compared to vehicle control group.

| Treatment Groups | Plasma HDL (mg/dL) |
|---|---|
| G1 | 116.28 ± 12.10 |
| G2 | 145.41 ± 11.48 |
| G3 | 110.76 ± 9.17 |

| Treatment Groups | Plasma LDL (mg/dL) |
|---|---|
| G1 | 17.97 ± 2.59 |
| G2 | 23.58 ± 2.93 |
| G3 | 14.78 ± 1.62 |

G2 and G3 showed decrease in liver weight and relative liver weight however the effect was not statistically significant.

| Treatment Groups | Absolute Liver Weight (g) |
|---|---|
| G1 | 2.46 ± 0.08 |
| G2 | 2.34 ± 0.07 |
| G3 | 2.34 ± 0.06 |

| Treatment Groups | Relative Liver Weight (g) |
|---|---|
| G1 | 0.060 ± 0.002 |
| G2 | 0.055 ± 0.001 |
| G3 | 0.056 ± 0.001 |

G2 and G3 showed decrease in epididymal adipose tissue weight and relative epididymal adipose tissue weight however the effect was nonsignificant compared to vehicle control group.

| Treatment Groups | Absolute Liver Weight (g) |
|---|---|
| G1 | 2.72 ± 0.01 |
| G2 | 2.42 ± 0.10 |
| G3 | 2.58 ± 0.07 |

| Treatment Groups | Relative Epididymal Tissue Weight (g) |
|---|---|
| G1 | 0.066 ± 0.002 |
| G2 | 0.057 ± 0.002 |
| G3 | 0.061 ± 0.001 |

G3 showed decrease in fasting plasma HbA1c however the effect was nonsignificant (p value=0.1043) compared to vehicle control group. G2 did not show significant change in fasting plasma HbA1c levels compared to vehicle control group.

| Treatment Groups | Fasting Plasma HbA1c (ng/mL) |
|---|---|
| G1 | 55.41 ± 8.03 |
| G2 | 60.13 ± 7.73 |
| G3 | 39.82 ± 4.31 |

| Treatment Groups | Fasting Plasma Insulin (ng/mL) |
|---|---|
| G1 | 4.7 ± 0.8 |
| G2 | 4.6 ± 0.5 |
| G3 | 5.2 ± 0.8 |

G3 showed decrease in fasting plasma fructosamine however the effect was nonsignificant compared to vehicle control group. G2 did not show significant change in fasting plasma fructosamine levels compared to vehicle control group.

| Treatment Groups | Fasting Fructosamine (ng/mL) |
|---|---|
| G1 | 1031.8 ± 98.1 |
| G2 | 1164.8 ± 105.5 |
| G3 | 954.2 ± 45.6 |

G2 and G3 plasma, liver and adipose tissue samples were collected post 4 hours dosing on day 36. Plasma drug concentration was in detectable range, however adipose tissue drug concentration was mostly not detectable and liver tissue drug concentration was not detectable.

| | | Plasma | Liver | Adipose tissue |
|---|---|---|---|---|
| PK concentration | | (ng/mL) | (ng/mL) | (ng/mL) |
| G2 | Average | 243.7 | BLQ | 19.5 |
| | SD | 94.0 | BLQ | 8.4 |
| | SEM | 29.7 | BLQ | 3.8 |
| G3 | Average | 196.7 | BLQ | 11.0 |
| | SD | 53.2 | BLQ | 6.7 |
| | SEM | 16.8 | BLQ | 3.9 |

### BIOLOGICAL EXAMPLE 13

### Cytotoxicity Assay

The CyQUANT Cytotoxicity Assay (Thermofisher, cat no: V23111) was used to detect damaged and dying cells by means of their release of glucose 6-phosphate dehydrogenase into the surrounding medium. Cells were plated in a 96-well microplate (500-25,000 cells/well in a 50-μL volume). Three sets of triplicate wells were reserved for the following controls: a no-cell control (medium alone), untreated cells, and completely lysed cells. Cells were starved for 2 h and then add the indicated AMPK activators (50 μL) to the experimental wells and incubate for 2 hours, 50 μL of 2× reaction mixture was added to each well. 1 μL of 100× Cell-Lysis buffer was added to the completely lysed control wells only. Cells were incubated at 37° C. for 10-30 minutes. Fluorescence was measured (excitation 530-560 nm, emission 580-600 nm) at 5-minute intervals to determine the optimal time point for the experiment.

### BIOLOGICAL EXAMPLE 14

### Induction of Glucose Uptake in HSKM and THLE2 Cells

To test AMPK activators, affect glucose uptake several compounds of structure (I) and (II) were tested. Tested compounds included compound I-1, compound I-10, compound I-11, compound I-7, compound I-13 and reference compounds MK-8722 and PF-06409577. Each representative compound of structure (I) increased glucose uptake in HSKM. Based on RLU values, compound I-11 appeared to increase much more glucose uptake compared with reference compounds indicating more potency. Compound II-10 also shows a dosage-dependent effect on glucose uptake. Consistently with HSKM cells, compound I-11 increases glucose uptake in dosage-dependent manner in human hepatocytes THLE-2 cells and human white adipocyte cells-HWP-c cells. For human cardiomyocyte cells, compound I-11 did not activate glucose uptake compared with reference compound PF-06409577, indicating compound I-11 did not have heart toxicity.

### BIOLOGICAL EXAMPLE 15

### AMPK Signal Pathway Study

Compounds I-11 and II-10 were tested for their effect on activation of AMPK signal pathways in skeletal muscle and liver cells. Namely, western blotting assays were used to confirm the glucose uptake assay results by detecting pAMPK/pACC/AMPKα2β1β2γ3 expression in human/mice/rat skeletal muscle cells-HSKM, C2C12, L6 cells.

In HSKM cells, compound II-10 and compound I-11 induced p-ACC and p-AMPK in dosage-dependent manner and show more potency activity compared with reference compound MK-8722. Meanwhile, compound II-10 increased AMPKα2β2γ3 expression, and compound I-11 increased AMPKα2β1γ3 expression. Consistent with HSKM, compounds II-10 and I-11 also induce pAMPK/pACC/AMPKα2β1β2γ3 expression in C2C12 cells (Western Blot analysis). In L6 cells, compounds II-10 and I-11 can also induce pAMPK/pACC/AMPKα2γ3 expression, the only difference is that β1β2 expression is quite low in L6. For human liver cells-HepG2, compounds II-10 and I-11 can activate p-AMPK/p-ACC and AMPKα2β1β2γ3 in dosage-dependent manner. For HWPC, compounds II-10 and I-11 did not activate AMPK signal pathway. The endogenous expression of AMPKα2 and AMPKγ3 are low, so it's hard to detect, which means these compounds likely did not affect adipocyte tissue-related AMPK isoforms. In human cardiac myocytes (HCM), compounds II-10 and I-11 did not activate p-ACC/p-AMPK and AMPKα2β2γ3 expression. MK-8722 may be able to increase p-ACC and p-AMPK and AMPKα2β2 expression, indicating that MK8722 may have cardiotoxicity relative to compounds II-10 and I-11.

After differentiation of cells (e.g., HSKM, C2C12, L6) for 5 days, medium was changed to DHEM without FBS and glucose for starving cells for 2 hours. After starving cells, the AMPK activators (i.e., compound I-11, compound II-10, MK-8722, and PF-06409577) for 1 hour. The resultant reaction mixture was then assayed and semi-quantitative histogram analysis of Western Blotting (signal value) was used to determine results.

### BIOLOGICAL EXAMPLE 16

### Toxicity Testing for AMPK Activators

To validate the toxicity of compounds I-11 and II-10, CyQUANT Cytotoxicity Assay Kit (G6PD assay) was used to compare compound toxicity to reference compounds MK-8722 and PF-06409577. Compared to reference compounds, compounds I-11 and II-10 have less toxicity in HSKM, HCM and HepG2 cells. This decreased toxicity indicates compounds of Structure (I) and (II) have less cellular toxicity in human muscle, human cardiovascular, and liver cells.

To text toxicity, after differentiation of human skeletal muscle cells, HSKM were starved with DMEM medium without FBS/glucose for 2 hours. After, cells were further treated with AMPK activators (i.e., compound I-11, compound II-10, MK-8722, and PF-06409577) for 2 hours. The resultant reaction mixture was then assayed for glucose 6-phosphate dehydrogenase release. An untreated control sample and completely lysed cells were detected for comparison. The fluorescence was measured in a microplate reader (excitation/emission ˜530/590 nm).

The various embodiments described above can be combined to provide further embodiments. All the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications, and publications to provide yet further embodiments.

These and other changes can be made to the embodiments considering the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A compound having the following Structure (I): 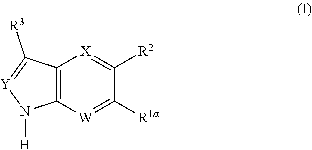
or a pharmaceutically acceptable salt, stereoisomer, or tautomer thereof, wherein:
Y is N or CH;
X is N or CR^{1b},
W is N or CR^{1c};
R^{1a}, R^{1b}, and R^{1c} are each independently hydrogen or halo;
R² is 3-8 membered N-heterocyclyl, —NH—R^{2d}, or has the following structure: 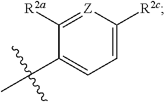
Z is N or CR^{2b},
R^{2a}, R^{2b}, and R^{2c} are each independently hydrogen, optionally substituted C₁-C₄ alkoxy, —(CH₂)ₙ₁O(CH₂)ₙ₂O(CH₂)ₙ₃OH, —N═S(CH₃)₂═O, optionally substituted C₃-C₈ cycloalkyl, or optionally substituted 3-8 membered heterocyclyl;
R^{2d} has one of the following structures: 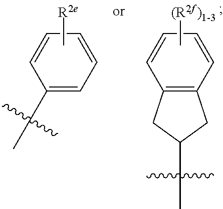
R^{2e} is optionally substituted C₃-C₆ cycloalkyl;
R^{2f} is halo or optionally substituted C₃-C₆ cycloalkyl;
n1, n2, and n3 are each independently 1, 2, 3, or 4; and
R³ has one of the following structures:

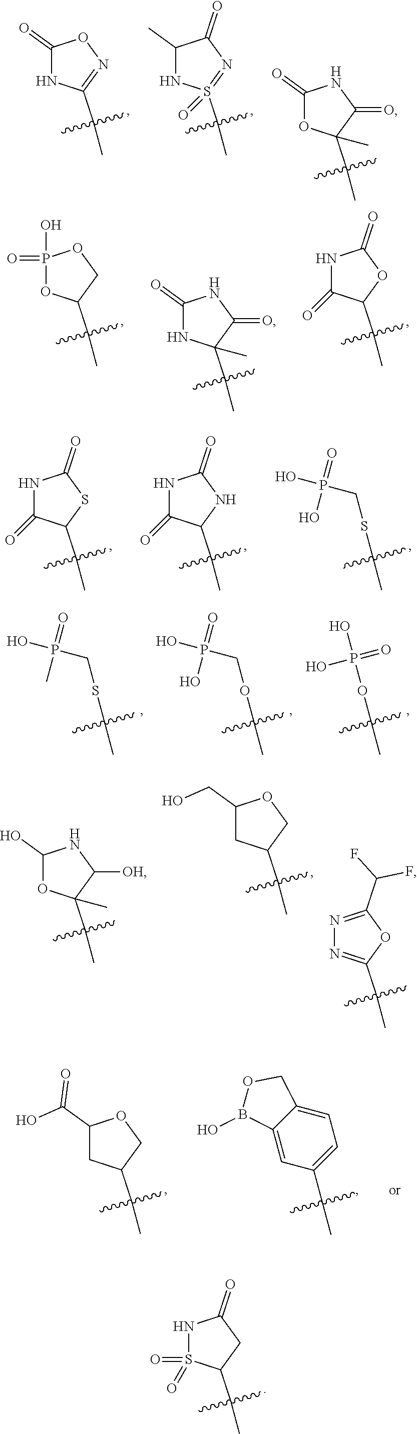

2-3. (canceled)

4. The compound of claim 1, wherein X is N, CH, or CF.

5. (canceled)

6. The compound of claim 1, wherein W is N or CH.

7. The compound of claim 1, wherein R^{1a} is hydrogen, fluoro, or chloro.

8. The compound of claim 1, wherein R² is a monocyclic 5-6 membered N-heterocyclyl.

9. The compound of claim 1, wherein R² is pyrrolidinyl.

10. The compound of claim 9, wherein R² is optionally substituted with one or more —OH substituents.

11. The compound of claim 1, wherein R² has one of the following structures:

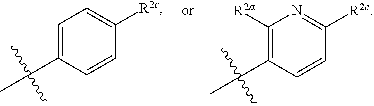

12. The compound of claim 1, wherein R^{2a} is methoxy.

13. The compound of claim 1, wherein R^{2c} is C₃-C₆ cycloalkyl optionally substituted with one or more —OH substituents.

14. The compound of claim 1, wherein R^{2c} is a 3-6 membered O-heterocyclyl or a 3-6 membered N-heterocyclyl each being optionally substituted with one or more —OH substituents.

15. The compound of claim 1, wherein R^{2a}, R^{2c}, or both are independently methoxy, —N═S(CH₃)₂═O, or have one of the following structures:

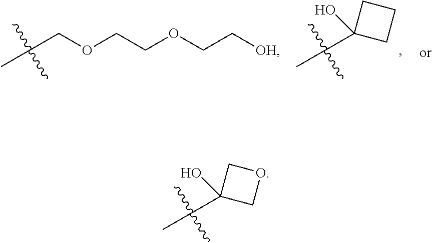 [SMILES: *COCCOCCO ; *C1(O)CCC1 ; "," ; "or" ; *C1(O)COC1]

16. The compound of claim 1, wherein R² has one of the following structures:

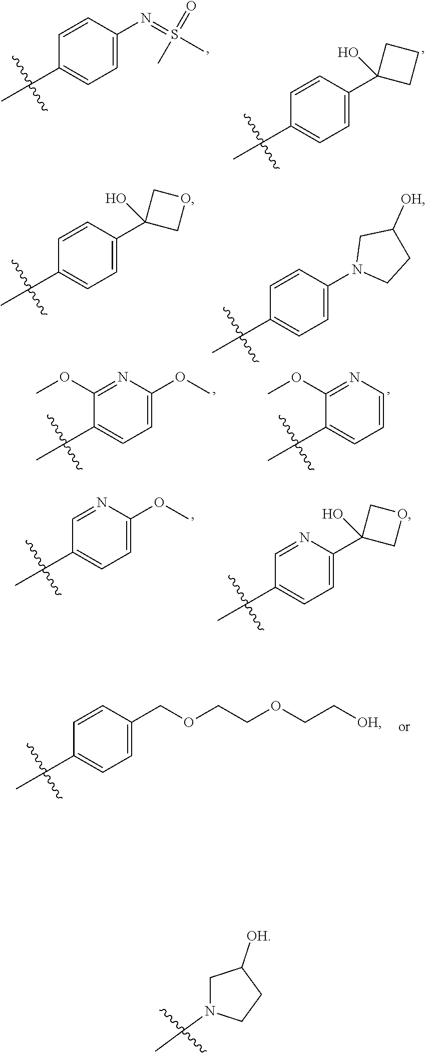

17. (canceled)

18. The compound of claim 1, wherein R² is —NH—R^{2d}.

19. The compound of claim 1, wherein R² has one of the following structures:

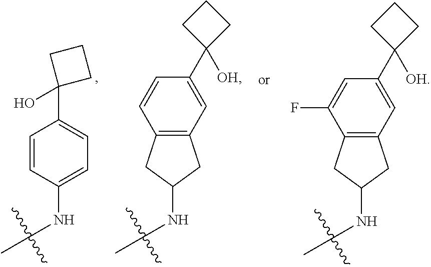 [SMILES: *Nc1ccc(C2(O)CCC2)cc1 ; "," ; *NC1Cc2ccc(C3(O)CCC3)cc2C1 ; "or" ; *NC1Cc2cc(C3(O)CCC3)cc(F)c2C1]

20. (canceled)

21. The compound of claim 1, wherein R³ has one of the following structures:

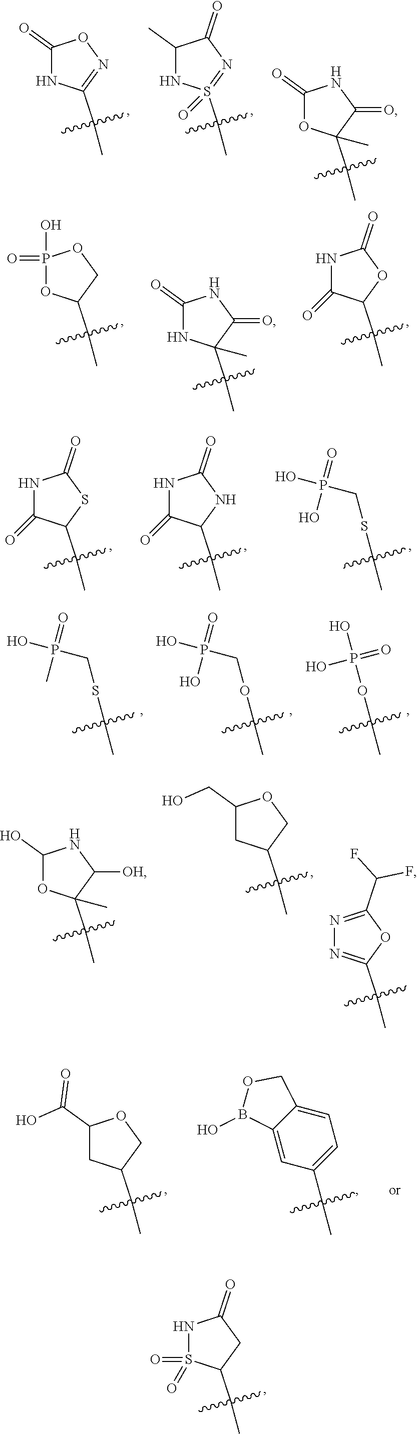

22-25. (canceled)

26. The compound of claim 1, wherein the compound has one of the following structures: 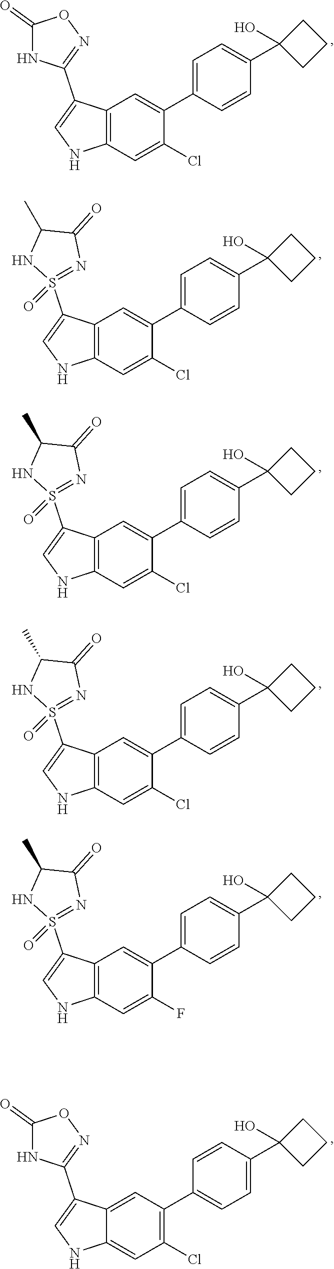 [SMILES: O=c1[nH]c(-c2c[nH]c3cc(Cl)c(-c4ccc(C5(O)CCC5)cc4)cc23)no1 ; CC1NS(=O)(c2c[nH]c3cc(Cl)c(-c4ccc(C5(O)CCC5)cc4)cc23)=NC1=O ; C[C@@H]1NS(=O)(c2c[nH]c3cc(Cl)c(-c4ccc(C5(O)CCC5)cc4)cc23)=NC1=O ; C[C@H]1NS(=O)(c2c[nH]c3cc(Cl)c(-c4ccc(C5(O)CCC5)cc4)cc23)=NC1=O ; C[C@@H]1NS(=O)(c2c[nH]c3cc(F)c(-c4ccc(C5(O)CCC5)cc4)cc23)=NC1=O ; O=c1[nH]c(-c2c[nH]c3cc(Cl)c(-c4ccc(C5(O)CCC5)cc4)cc23)no1] 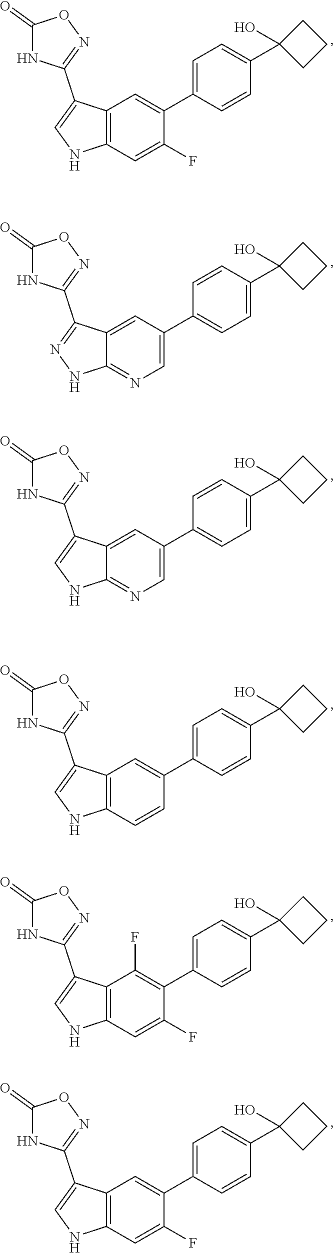 [SMILES: O=c1[nH]c(-c2c[nH]c3cc(F)c(-c4ccc(C5(O)CCC5)cc4)cc23)no1 ; O=c1[nH]c(-c2n[nH]c3ncc(-c4ccc(C5(O)CCC5)cc4)cc23)no1 ; O=c1[nH]c(-c2c[nH]c3ncc(-c4ccc(C5(O)CCC5)cc4)cc23)no1 ; O=c1[nH]c(-c2c[nH]c3ccc(-c4ccc(C5(O)CCC5)cc4)cc23)no1 ; O=c1[nH]c(-c2c[nH]c3cc(F)c(-c4ccc(C5(O)CCC5)cc4)c(F)c23)no1 ; O=c1[nH]c(-c2c[nH]c3cc(F)c(-c4ccc(C5(O)CCC5)cc4)cc23)no1] 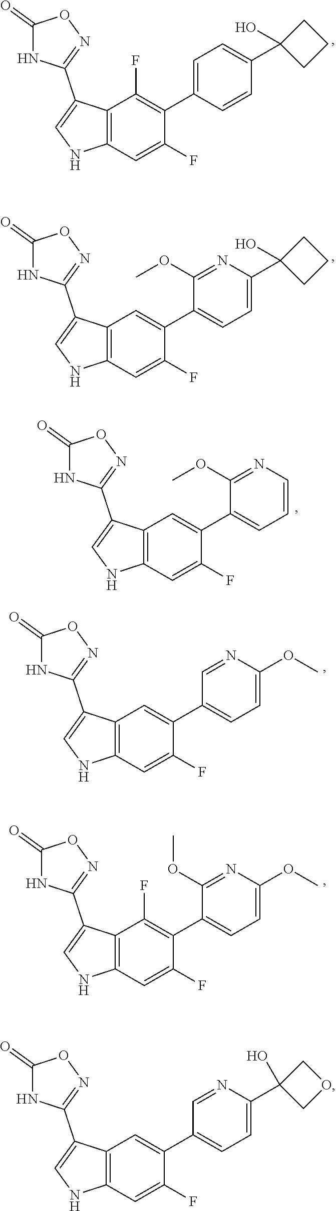 [SMILES: O=c1[nH]c(-c2c[nH]c3cc(F)c(-c4ccc(C5(O)CCC5)cc4)c(F)c23)no1 ; COc1nc(C2(O)CCC2)ccc1-c1cc2c(-c3noc(=O)[nH]3)c[nH]c2cc1F ; COc1ncccc1-c1cc2c(-c3noc(=O)[nH]3)c[nH]c2cc1F ; COc1ccc(-c2cc3c(-c4noc(=O)[nH]4)c[nH]c3cc2F)cn1 ; COc1ccc(-c2c(F)cc3[nH]cc(-c4noc(=O)[nH]4)c3c2F)c(OC)n1 ; O=c1[nH]c(-c2c[nH]c3cc(F)c(-c4ccc(C5(O)COC5)nc4)cc23)no1] 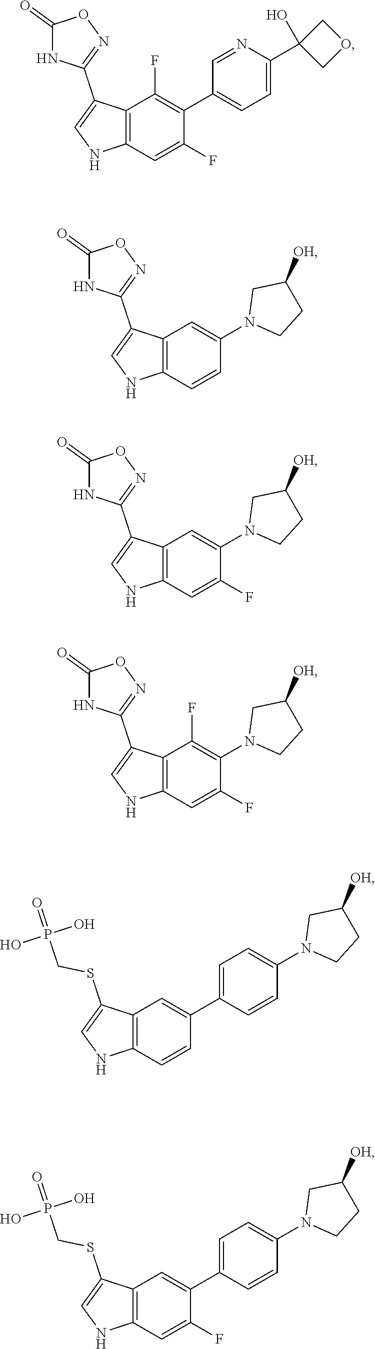 [SMILES: O=c1[nH]c(-c2c[nH]c3cc(F)c(-c4ccc(C5(O)COC5)nc4)c(F)c23)no1 ; O=c1[nH]c(-c2c[nH]c3ccc(N4CC[C@H](O)C4)cc23)no1 ; O=c1[nH]c(-c2c[nH]c3cc(F)c(N4CC[C@H](O)C4)cc23)no1 ; O=c1[nH]c(-c2c[nH]c3cc(F)c(N4CC[C@H](O)C4)c(F)c23)no1 ; O=P(O)(O)CSc1c[nH]c2ccc(-c3ccc(N4CC[C@H](O)C4)cc3)cc12 ; O=P(O)(O)CSc1c[nH]c2cc(F)c(-c3ccc(N4CC[C@H](O)C4)cc3)cc12] 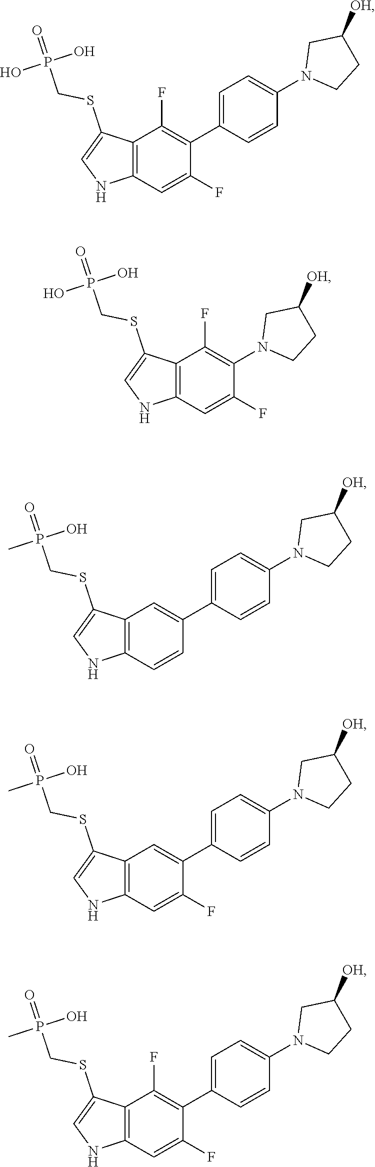 [SMILES: O=P(O)(O)CSc1c[nH]c2cc(F)c(-c3ccc(N4CC[C@H](O)C4)cc3)c(F)c12 ; O=P(O)(O)CSc1c[nH]c2cc(F)c(N3CC[C@H](O)C3)c(F)c12 ; CP(=O)(O)CSc1c[nH]c2ccc(-c3ccc(N4CC[C@H](O)C4)cc3)cc12 ; CP(=O)(O)CSc1c[nH]c2cc(F)c(-c3ccc(N4CC[C@H](O)C4)cc3)cc12 ; CP(=O)(O)CSc1c[nH]c2cc(F)c(-c3ccc(N4CC[C@H](O)C4)cc3)c(F)c12] 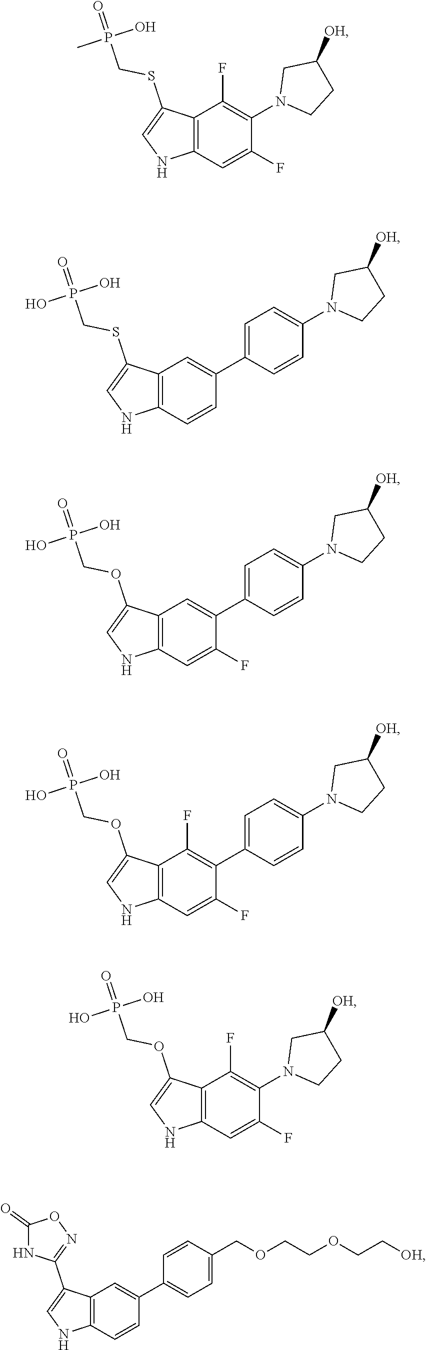 [SMILES: CP(=O)(O)CSc1c[nH]c2cc(F)c(N3CC[C@H](O)C3)c(F)c12 ; O=P(O)(O)CSc1c[nH]c2ccc(-c3ccc(N4CC[C@H](O)C4)cc3)cc12 ; O=P(O)(O)COc1c[nH]c2cc(F)c(-c3ccc(N4CC[C@H](O)C4)cc3)cc12 ; O=P(O)(O)COc1c[nH]c2cc(F)c(-c3ccc(N4CC[C@H](O)C4)cc3)c(F)c12 ; O=P(O)(O)COc1c[nH]c2cc(F)c(N3CC[C@H](O)C3)c(F)c12 ; O=c1[nH]c(-c2c[nH]c3ccc(-c4ccc(COCCOCCO)cc4)cc23)no1] 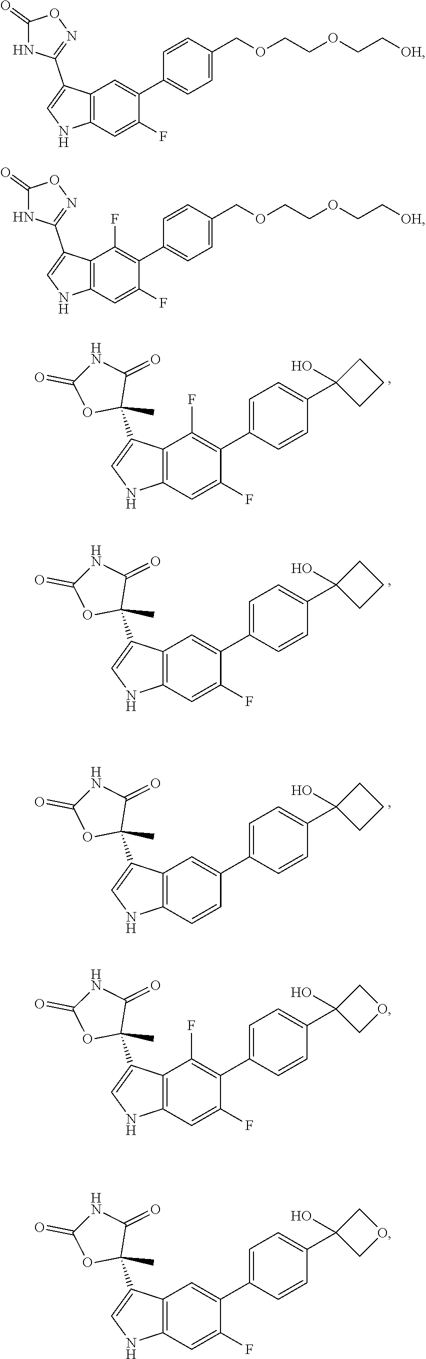 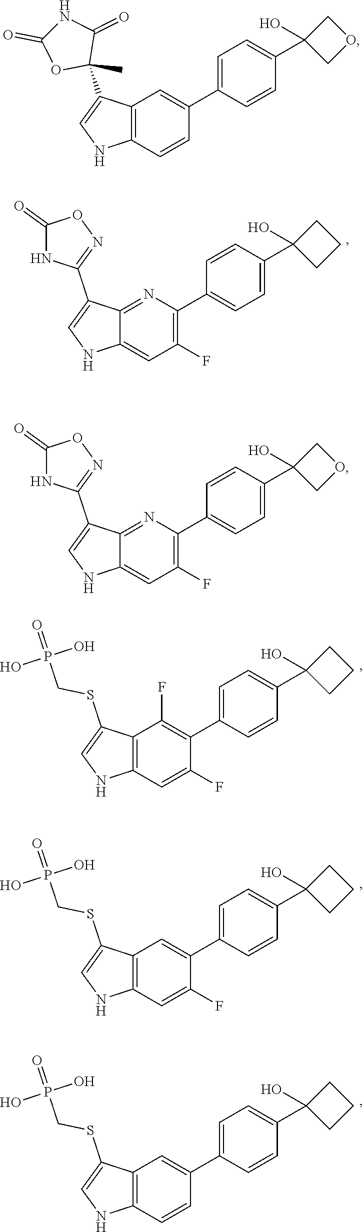 [SMILES: C[C@@]1(c2c[nH]c3ccc(-c4ccc(C5(O)COC5)cc4)cc23)OC(=O)NC1=O ; O=c1[nH]c(-c2c[nH]c3cc(F)c(-c4ccc(C5(O)CCC5)cc4)nc23)no1 ; O=c1[nH]c(-c2c[nH]c3cc(F)c(-c4ccc(C5(O)COC5)cc4)nc23)no1 ; O=P(O)(O)CSc1c[nH]c2cc(F)c(-c3ccc(C4(O)CCC4)cc3)c(F)c12 ; O=P(O)(O)CSc1c[nH]c2cc(F)c(-c3ccc(C4(O)CCC4)cc3)cc12 ; O=P(O)(O)CSc1c[nH]c2ccc(-c3ccc(C4(O)CCC4)cc3)cc12] 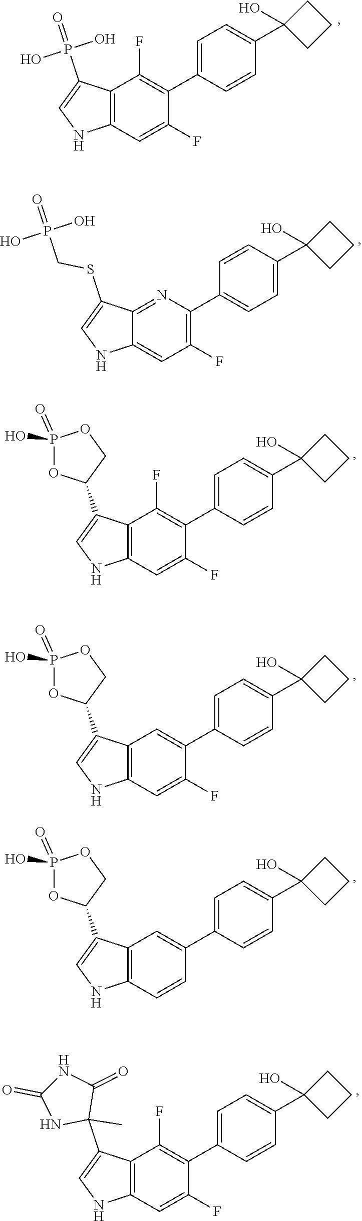 [SMILES: O=P(O)(O)c1c[nH]c2cc(F)c(-c3ccc(C4(O)CCC4)cc3)c(F)c12 ; O=P(O)(O)CSc1c[nH]c2cc(F)c(-c3ccc(C4(O)CCC4)cc3)nc12 ; O=[P@]1(O)OC[C@H](c2c[nH]c3cc(F)c(-c4ccc(C5(O)CCC5)cc4)c(F)c23)O1 ; O=[P@]1(O)OC[C@H](c2c[nH]c3cc(F)c(-c4ccc(C5(O)CCC5)cc4)cc23)O1 ; O=[P@]1(O)OC[C@H](c2c[nH]c3ccc(-c4ccc(C5(O)CCC5)cc4)cc23)O1 ; CC1(c2c[nH]c3cc(F)c(-c4ccc(C5(O)CCC5)cc4)c(F)c23)NC(=O)NC1=O] 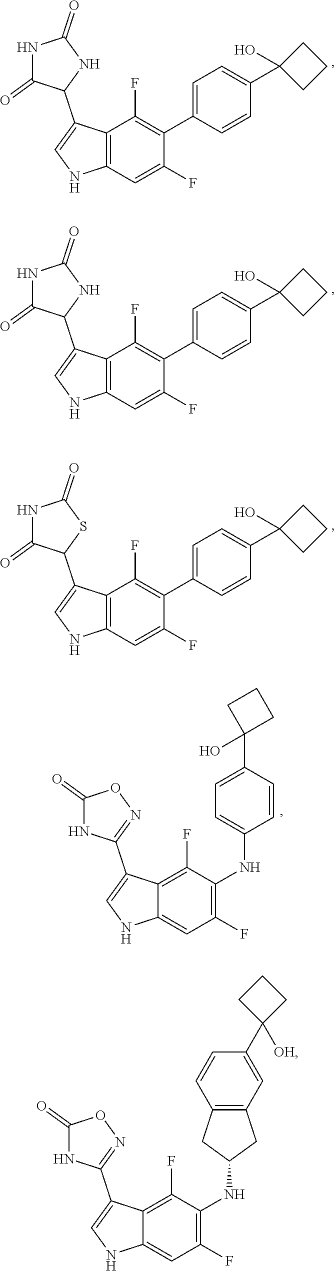 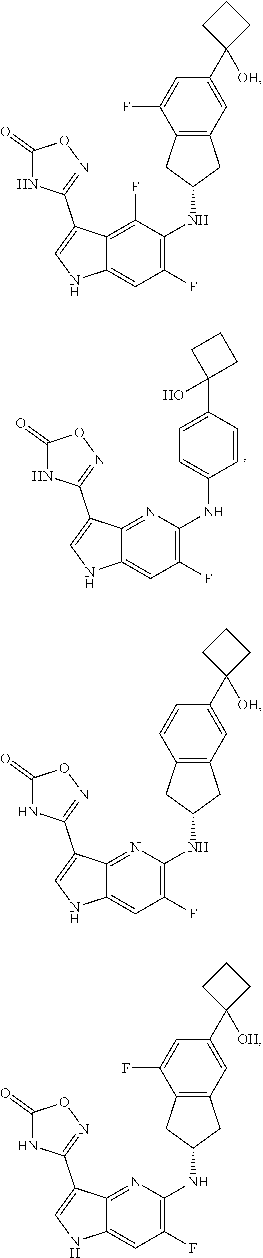 [SMILES: O=c1[nH]c(-c2c[nH]c3cc(F)c(N[C@H]4Cc5cc(C6(O)CCC6)cc(F)c5C4)c(F)c23)no1 ; O=c1[nH]c(-c2c[nH]c3cc(F)c(Nc4ccc(C5(O)CCC5)cc4)nc23)no1 ; O=c1[nH]c(-c2c[nH]c3cc(F)c(N[C@@H]4Cc5ccc(C6(O)CCC6)cc5C4)nc23)no1 ; O=c1[nH]c(-c2c[nH]c3cc(F)c(N[C@H]4Cc5cc(C6(O)CCC6)cc(F)c5C4)nc23)no1] 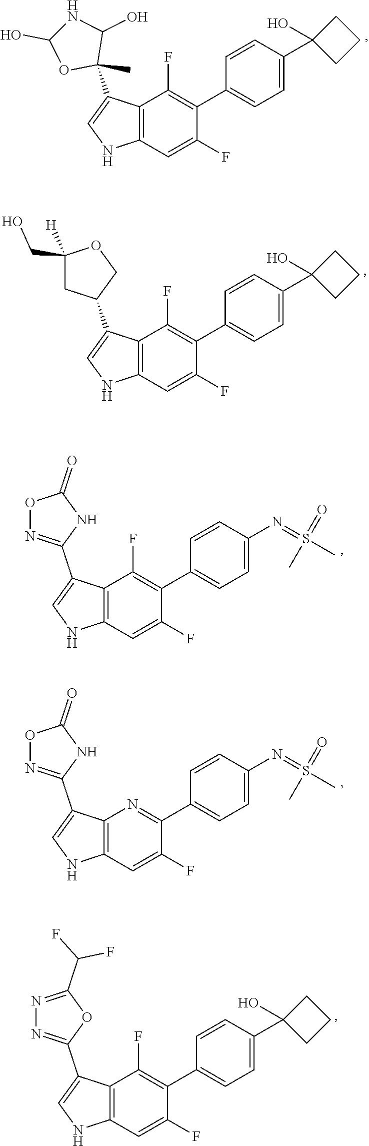 [SMILES: C[C@@]1(c2c[nH]c3cc(F)c(-c4ccc(C5(O)CCC5)cc4)c(F)c23)OC(O)NC1O ; OC[C@@H]1C[C@@H](c2c[nH]c3cc(F)c(-c4ccc(C5(O)CCC5)cc4)c(F)c23)CO1 ; CS(C)(=O)=Nc1ccc(-c2c(F)cc3[nH]cc(-c4noc(=O)[nH]4)c3c2F)cc1 ; CS(C)(=O)=Nc1ccc(-c2nc3c(-c4noc(=O)[nH]4)c[nH]c3cc2F)cc1 ; OC1(c2ccc(-c3c(F)cc4[nH]cc(-c5nnc(C(F)F)o5)c4c3F)cc2)CCC1] 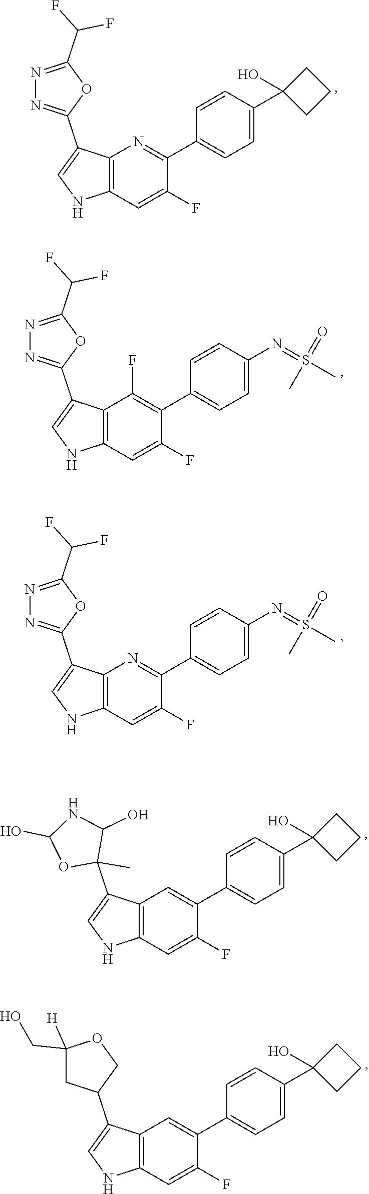 [SMILES: OC1(c2ccc(-c3nc4c(-c5nnc(C(F)F)o5)c[nH]c4cc3F)cc2)CCC1 ; CS(C)(=O)=Nc1ccc(-c2c(F)cc3[nH]cc(-c4nnc(C(F)F)o4)c3c2F)cc1 ; CS(C)(=O)=Nc1ccc(-c2nc3c(-c4nnc(C(F)F)o4)c[nH]c3cc2F)cc1 ; CC1(c2c[nH]c3cc(F)c(-c4ccc(C5(O)CCC5)cc4)cc23)OC(O)NC1O ; OCC1CC(c2c[nH]c3cc(F)c(-c4ccc(C5(O)CCC5)cc4)cc23)CO1] 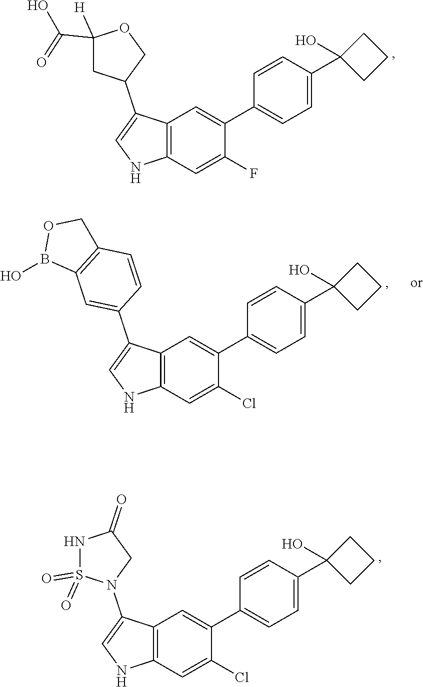 [SMILES: O=C(O)C1CC(c2c[nH]c3cc(F)c(-c4ccc(C5(O)CCC5)cc4)cc23)CO1 ; OB1OCc2ccc(-c3c[nH]c4cc(Cl)c(-c5ccc(C6(O)CCC6)cc5)cc34)cc21 ; O=C1CN(c2c[nH]c3cc(Cl)c(-c4ccc(C5(O)CCC5)cc4)cc23)S(=O)(=O)N1]
or a pharmaceutically acceptable salt, stereoisomer, or tautomer thereof.

27. A compound of having the following Structure (II): 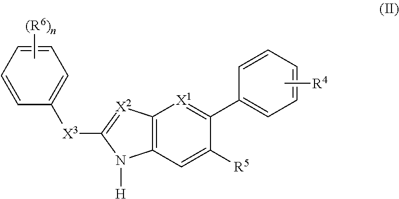
or a pharmaceutically acceptable salt, stereoisomer, or tautomer thereof, wherein:
X¹ is N or CR⁷;
X² is N or CH;
X³ is —NH—, —O—, or —S—;
R⁴ is optionally substituted C₃-C₆ cycloalkyl, —N═S(R^{4a})₂═O, or optionally substituted phenyl, wherein R^{4a} is, at each occurrence, independently C₁-C₄ alkyl;
R⁵ is hydrogen or halo;
R⁶ is, at each occurrence, independently optionally substituted C₁-C₄ alkyl, optionally substituted 5-membered heterocyclyl, or
two occurrences of Re join, together with the carbons to which they are attached to form a 4-6 membered heterocyclyl;
R⁷ is hydrogen or halo; and
nis 0, 1, 2, or 3.

28-62. (canceled)

63. A salt form of the compound of claim 1, wherein the salt form is a formic acid salt, a hydrochloric acid salt, or a trifluoroacetic acid salt.

64. A pharmaceutical composition comprising the compound of claim 1, or a pharmaceutically acceptable salt, stereoisomer, or tautomer thereof, and a pharmaceutically acceptable excipient.

65. A method of treating an AMPK mediated disease, the method comprising administering the compound of claim 1, or a pharmaceutically acceptable salt, stereoisomer, or tautomer thereof, wherein the AMPK mediated disease is a cardiometabolic disease, a neuromuscular disorder, a cancer, a neurodegenerative disease, or combinations thereof.

66. (canceled)

67. The method of claim 65, wherein the AMPK mediated disease is obesity, diabetes, chronic inflammation, cardiac energy homeostasis, ischemia-reperfusion injury, endothelial dysfunction, dyslipidemia, cardiac hypertrophy and remodeling, Duchenne muscular dystrophy (DMD), myotonic dystrophy type 1 (DM1), spinal muscular atrophy (SMA), Non-alcoholic Fatty Liver Disease (NAFLD), Alzheimer's disease, or combinations thereof.
